(19) United States
(12) Patent Application Publication
(10) Pub. No.: US 2023/0365559 A1
(43) Pub. Date: Nov. 16, 2023

(54) NOVEL AMINOPYRIDINES AND THEIR USE IN TREATING CANCER

(71) Applicant: Auckland UniServices Limited, Auckland (NZ)

(72) Inventors: Benjamin D. DICKSON, Auckland (NZ); Michael Patrick HAY, Auckland (NZ); Cho Rong HONG, Auckland (NZ); Way Wua WONG, Auckland (NZ); William Robert WILSON, Auckland (NZ); Lydia Pieng Ping LIEW, Auckland (NZ); Stephen Michael Frazer JAMIESON, Auckland (NZ)

(73) Assignee: Auckland UniServices Limited, Auckland (NZ)

(21) Appl. No.: 18/246,100

(22) PCT Filed: Sep. 24, 2021
(86) PCT No.: PCT/IB2021/058707

§ 371 Date: Mar. 21, 2023

(30) Foreign Application Priority Data
Sep. 24, 2020 (NZ) 768245

(51) Int. Cl. C07D 471/04; C07D 519/00; A61P 35/00

(52) U.S. Cl. CPC C07D 471/04; C07D 519/00; A61P 35/00

(57) ABSTRACT

The invention relates to substituted imidazo[4,5-c]pyridine-2-one compounds of Formula (I) and prodrugs of said compounds. Compounds of Formula (I) selectively inhibit the activity of DNA-dependent protein kinase (DNA-PK) and are therefore useful in the treatment of diseases in which inhibition of DNA-PK is beneficial.

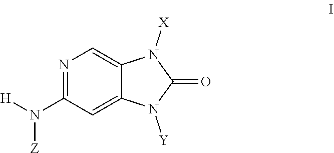

### CROSS-REFERENCE TO AND BENEFIT OF EARLIER APPLICATIONS

This is a U.S. national phase application under 35 U.S.C. § 371 of United States Patent Cooperation Treaty Application No. PCT/IB2021/058707, filed Sep. 24, 2021, which claims priority from New Zealand Patent Application Serial No. 768245, filed on Sep. 24, 2020, and this national phase application incorporates by reference those PCT and New Zealand patent applications in their entireties.

### 1. FIELD OF THE INVENTION

The invention generally relates to substituted imidazo[4,5-c]pyridine-2-one compounds, prodrugs and pharmaceutically acceptable salts thereof. These compounds selectively inhibit the activity of DNA-dependent protein kinase (DNA-PK). The invention also relates to the use of these compounds, prodrugs, salts and solvates thereof to treat diseases that are modulated by DNA-PK, including cancer. The invention also relates to pharmaceutical preparations of substituted imidazo[4,5-c]pyridine-2-one compounds.

### 2. BACKGROUND

Cancer treatment is still dominated by the use of cytotoxic agents, including therapies such as ionising radiation and topoisomerase inhibitors which produce DNA double strand breaks (DSBs) as the principal cytotoxic lesions. All cells have highly organised DNA damage responses (DDR) that include repair of DNA damage. Two principal repair mechanisms deal with DNA DSBs. Homologous recombination repair (HRR) uses a sister chromatid to effect high fidelity repair in S and G2 phases of the cell cycle, while non-homologous end-joining (NHEJ) results in error-prone rejoining of chromosomes throughout the cell cycle. These repair mechanisms engender resistance to cytotoxic chemotherapy and radiotherapy. Conversely, loss of function in a particular DDR pathway may sensitise cancer cells to a particular cytotoxic agent through the persistence of DNA lesions. Therapeutic targeting of the DDR to augment the activity of conventional chemotherapy, and to overcome resistance, has an extensive history.

Inactivation of elements of the NHEJ pathway results in highly radiosensitive phenotypes, implicating NHEJ as the dominant repair pathway for radiation-induced DSBs. Central to NHEJ is the DNA-dependent protein kinase (DNA-PK) complex. This comprises the proteins Ku70 and Ku80, which bind the free DNA ends at DSBs and recruit the DNA-PK catalytic subunit (DNA-PKcs). The resulting complex autophosphorylates DNA-PKcs (on Ser2056) and multiple other targets, resulting in its dissociation from the DNA and recruitment of Artemis, the XRCC4 complex, specialised DNA polymerases and DNA ligase 4 to rejoin the break. Loss of DNA-PK function results in severe sensitivity to DSBs and DNA-PK has been identified as a credible drug target in the DDR.

However, selectivity for DNA-PK over other protein kinases is an issue. DNA-PKcs, Ataxia telangiectasia-mutated (ATM), ATM-related (ATR) and mammalian target of rapamycin (mTOR) are members of the PI3K-related kinase (PIKK) family. They share homology with PI3K enzymes, but are protein (Ser/Thr) rather than lipid kinases. To be clinically useful, a new DNA-PK inhibitor must demonstrate at least some selectivity for DNA-PK compared to PI3K isoforms and other PIKK family members.

DNA-PK also possesses functions outside of its canonical role in the DSB repair. It has been reported to play roles in responses to glucose deprivation, hypoxia tolerance, myogenic differentiation, endothelial cell function, vascular smooth muscle proliferation, neuroprotection, mitosis, telomere protection and regulation of inflammatory and immune responses. Thus, normal tissue toxicities of DNA-PK inhibitors are not unexpected.

The use of DDR inhibitors in combination with DNA damaging chemotherapy has been plagued by the enhancement of normal tissue toxicity, requiring reductions in the chemotherapy dose and compromising efficacy. This suggests that the combination of DNA-PK inhibition with radiotherapy is a promising opportunity. Concerns about potentiation of normal tissue toxicity within the radiation field have been offset, to some extent, by the advent of highly conformal radiotherapy techniques such as intensity modulated radiation therapy (IMRT), image-guided radiation therapy (IGRT) and stereotactic body radiotherapy (SBRT).

Tumour selective drug delivery to hypoxic areas within solid tumours has the potential to provide another layer of selectivity to minimise normal tissue toxicity. Hypoxia is a cardinal element of the tumour microenvironment. It plays a dynamic role in tumour progression and determining responses to treatment. Hypoxia has also been shown to down-regulate Rad51 and BRCA1 and to limit HRR repair in hypoxic cells, increasing dependence on NHEJ. Hypoxic cells contribute to resistance to therapy, particularly radiotherapy and targeting these cells provides clinical benefit. Hypoxia-activated prodrugs are activated through enzymatic reduction in hypoxic tissue to release the active agents which may diffuse to adjacent tumour tissue (a local bystander effect).

Accordingly, while targeting of the DDR has considerable potential for the treatment of cancer, there is a need for new inhibitors of DNA-PK and/or effective hypoxia-activated prodrugs to deliver these compounds to tumours. It is therefore an object of the invention to go at least some way towards meeting this need, or at least to provide the public with a useful choice.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### 3. SUMMARY OF THE INVENTION

The inventors have unexpectedly determined that certain imidazo[4,5-c]pyridin-2-one compounds have high selectivity for DNA-PK and show activity as inhibitors of DNA-PK in vivo.

Accordingly, these compounds can be used to treat diseases that benefit from inhibition of this enzyme, such as cancer.

The invention also relates to novel nitroheteroaryl prodrugs of this class that are active against radioresistant (hypoxic) tumour cells in vivo.

Accordingly, the invention provides a compound of any one of Formulae I, II, III, IV, V, VI, VII or XII or a salt thereof, as set out below.

In another aspect the invention provides a pharmaceutical composition comprising a compound of Formula I, II, III, IV, V, VI or VII, or a salt or solvate thereof, in combination with one or more pharmaceutically acceptable excipients.

In another aspect the invention provides a method for treating a disease in which inhibition of DNA-PK is beneficial in a subject in need thereof, the method comprising administering to the subject a therapeutically effective amount of a compound of Formula I, II, III, IV, V, VI or VII or a pharmaceutically acceptable salt or solvate thereof.

In another aspect the invention provides a use of a compound of Formula I, II, III, IV, V, VI or VII, or a pharmaceutically acceptable salt or solvate thereof, in the manufacture of a medicament for the treatment of a disease in which inhibition of DNA-PK is beneficial.

In another aspect the invention provides a compound of Formula I, II, III, IV, V, VI or VII, or a pharmaceutically acceptable salt or solvate thereof, for use in the treatment of a disease in which inhibition of DNA-PK is beneficial.

In one embodiment, the disease in which inhibition of DNA-PK is beneficial is cancer.

In another aspect the invention provides a method of inhibiting DNA-PK mediated phosphorylation of a peptide substrate, the method comprising contacting the peptide substrate with an effective amount of a compound of any one of Formulae I, II, III, IV, V, VI or VII, or a pharmaceutically acceptable salt or solvate thereof.

In another aspect the invention provides a method of radiosensitizing a tumour cell comprising contacting the tumour cell with an effective amount of a compound of any one of Formulae I, II, III, IV, V, VI or VII, or a pharmaceutically acceptable salt or solvate thereof.

In another aspect the invention provides a method of inhibiting tumour growth comprising contacting the tumour with an effective amount of compound of any one of Formulae I, II, III, IV, V, VI or VII, or a pharmaceutically acceptable salt or solvate thereof.

In another aspect the invention provides a method for treating cancer, the method comprising administering to the subject a therapeutically effective amount of a compound of any one of Formulae I, II, III, IV, V, VI or VII, or a pharmaceutically acceptable salt or solvate thereof, in combination with radiotherapy, wherein the compound of any one of Formulae I, II, III, IV, V, VI or VII, or a pharmaceutically acceptable salt or solvate thereof, is administered simultaneously, separately or sequentially with the radiotherapy.

In one embodiment the radiotherapy is selected from the group consisting of IMRT, FRT, SBRT, SABR and IORT.

In one embodiment, the cancer is head and neck squamous cell carcinoma.

Although the present invention is broadly as defined above, those persons skilled in the art will appreciate that the invention is not limited thereto, and that the invention also includes embodiments of which the following description gives examples.

### 4. BRIEF DESCRIPTION OF THE FIGURES

The invention will now be described with reference to the accompanying Figures in which:

FIG. 1 is a histogram of kinase inhibition for compound 48 showing kinase inhibition by compound 48 at 1 μM (mean of duplicates, 10 μM ATP) ranked in descending order. Shade represents kinase family, DNA-PK indicated. Inhibition of kinase activity was evaluated by Reaction Biology Corporation (Malvern, PA) with the HotSpot assay platform, against a panel of 397 protein kinases and 20 lipid kinases.

FIG. 2 is a histogram of kinase inhibition for compound 121 showing kinase inhibition by compound 121 at 1 μM (mean of duplicates, 10 μM ATP) ranked in descending order. Shade represents kinase family, DNA-PK indicated. Inhibition of kinase activity was evaluated by Reaction Biology Corporation (Malvern, PA) with the HotSpot assay platform, against a panel of 397 protein kinases and 20 lipid kinases.

FIG. 3 is a series of graphs showing the radiosensitisation of UT-SCC-54C head and neck squamous cell carcinoma cells by compounds of the invention under aerobic conditions, determined by regrowth assay. UT-SCC-54C cells were seeded in 96-well plates (0.2 mL/well) with 200 and 800 cells (unirradiated and 3 Gy plates, respectively), exposed to compounds for 1 h before, during and for 18 h after irradiation (3 Gy), then regrown in fresh medium for 5 days before staining with sulforhodamine B. Controls were treated identically without irradiation (0 Gy). Regrowth fractions are normalised to the no-drug values. Values are means for two biological replicates.

FIG. 4 is a series of graphs showing the radiosensitisation of HAP1 and HAP1/PRKDC^{-/-} cells by compounds of the invention under aerobic conditions, determined by regrowth assay. Cells were seeded in 96-well plates, exposed to compounds for 1 h before, during and for 18 h after irradiation (3 Gy), then regrown in fresh medium for 5 days before staining with sulforhodamine B. Controls were treated identically without irradiation (0 Gy). Regrowth fractions are normalised to the no-drug values.

FIG. 5 is an immunoblot showing inhibition of cellular DNA-PK autophosphorylation, assessed by western immunoblotting of Ser2056 of DNA-PKcs, under oxic conditions. Lysates were prepared 30 min after the midpoint of the irradiation. UT-SCC-54C cells were exposed to 20 μM 48 or 88 (DNA-PK inhibitor) or 234 (prodrug) and irradiated 3 h later. Percent inhibition relative to control is expressed above each lane.

FIG. 6 is an immunoblot showing inhibition of cellular DNA-PK autophosphorylation, assessed by western immunoblotting of Ser2056 of DNA-PKcs, under anoxic conditions. Lysates were prepared 30 min after the midpoint of the irradiation. UT-SCC-54C cells were exposed to 20 μM 48 or 88 (DNA-PK inhibitor) or 234 (prodrug) and irradiated 3 h later. Percent inhibition relative to control is expressed above each lane.

FIG. 7 is an immunoblot showing inhibition of cellular autophosphorylation at Ser2056 of DNA-PKcs or Ser1981 of ataxia-telangiectasia mutated (ATM) in UT-SCC-54C cells assessed by western immunoblotting 30 min after the midpoint of the irradiation. UT-SCC-54C cells were exposed to 10 μM AZD1390 (ATM inhibitor), 48 (DNA-PK inhibitor), 234 (prodrug), 195 (DNA-PK inhibitor), 121 (DNA-PK inhibitor), M3814 (DNA-PK inhibitor) or IC87361 (DNA-PK inhibitor) under oxic conditions and irradiated 3 h later.

FIG. 8 is a series of graphs showing radiosensitisation of UT-SCC-54C tumour cells. Radiation survival curves for monolayers exposed to compounds 48, 121, 125, 135 and 195. UT-SCC-54C cells were exposed to compounds for 3 h before and during irradiation under oxia then held under oxic conditions for a further 18 h before trypsinising, counting and plating for clonogenic assay. Points are means of two biological replicates in a single experiment. Surviving fractions (SF) were calculated as the plating efficiency (PE) of compound+radiation divided by the plating efficiency of the compound alone: SF=PE_{(compound+RAD)}/PE_{(compound only)} (See Table 18 for full data). Lines are fits to the linear-quadratic model.

FIG. 9 is a series of graphs showing radiosensitisation of UT-SCC-54C tumour cells under oxic and anoxic conditions. UT-SCC-54C cells were exposed to compounds for 3 h before and during irradiation under oxia or anoxia, then held under oxic conditions for 18 h before trypsinising, counting and plating for clonogenic assay. SF=PE_{(compound+RAD)}/PE_{(compound only)} (See Table 18 for full data). Points are means of two biological replicates in a single experiment. Lines are fits to the linear-quadratic model.

FIG. 10 is a series of graphs showing radiosensitisation of UT-SCC-54C tumour cells under anoxic conditions. Sensitiser enhancement ratios at 10% surviving fraction (SER₁₀) and radiation doses for 10% (D₁₀) were determined for triplicate clonogenic survival assays of compounds 88, 121 and 135 and their respective prodrugs 234, 248 and 236 in UT-SCC-54C cells under anoxia. SER were also determined for clonogenic survival assays of compounds 121, 135 and 121 and their respective prodrugs 251, 238 and 250 in UT-SCC-54C cells under anoxia.

FIG. 11 is a plot showing radiosensitisation of UT-SCC-54C HNSCC tumours. Survival of tumour clonogens 18 h after dosing of female NIH-III mice bearing subcutaneous UT-SCC-54C tumours with compound 121 or compound 248 alone or in combination with 13 Gy whole body radiation (RAD). Mice were dosed intraperitoneally (IP) with compounds (50 mg/kg) 15 min before and 6 h after irradiation. Tumours were excised 18 h later, dissociated and plated for clonogenic assay. Points are clonogens per gram of tumour tissue for individual mice and horizontal bars are the means of the log-transformed values. Using two-way ANOVA with radiation and drug treatment as factors, the effect of radiation alone was highly significant (P<0.001) and the effect of compound 121 and the prodrug 248 in combination with radiation were significant relative to radiation alone, (P=0.005 and P=0.018, respectively).

FIG. 12 is a pair of graphs showing radiosensitisation of UT-SCC-54C HNSCC tumours. Inhibition of tumour growth after dosing of female NIH-III mice bearing subcutaneous UT-SCC-54C tumours with compound 121 alone or in combination with 10 Gy targeted radiation (RAD). Mice (eight per group) were dosed orally (PO) with 121 (100 or 400 mg/kg) 15 min before and 3 h after irradiation. Tumour volumes were measured until they exceeded 4× volume at treatment. Differences between groups in the time to endpoint were assessed by the Log-Rank test with Holm-Sidak multiple comparison analysis. The combination of DNA-PK inhibitor 121 with radiation provided considerable extension in time to relative tumour volume 4× original compared to 121 or radiation alone.

FIG. 13 is a series of graphs showing the comparative selectivity data for 121 and AZD7648. Each pair represents an independent comparison. The pIC50 values (−log IC50) were determined in biochemical assays against DNA-PK and mTOR and related PI3K isoforms by Reaction Biology Corp (Malvern, PA). The fold selectivity for DNA-PK as IC50(enzyme)/IC50(DNA-PK) is shown above each bar for the enzymes.

FIG. 14 is a plot showing metabolism of prodrug 248 by UT-SCC-54C cells under anoxic conditions, but not under oxic conditions. Selective metabolism of 248 releases the DNA-PK inhibitor 121. Concentrations of 121 and 248 were quantified by LC-MS and values are means±SE from 3 biological replicates.

### 5. DETAILED DESCRIPTION OF THE INVENTION

The details of the invention are set forth in the accompanying description below.

Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entirety.

5.1 Definitions

As used in the present specification, the following words, phrases and symbols are generally intended to have the meanings set forth below, except to the extent that the context in which they are used indicates otherwise.

The term “comprising” as used herein means “consisting at least in part of”. When interpreting each statement in this specification and claims that includes the term “comprising”, features other than that or those prefaced by the term may also be present. Related terms such as “comprise” and “comprises” are to be interpreted in the same manner.

As used herein the term “and/or” means “and” or “or”, or both.

Where the term “optionally” is used, it is intended that the subsequent feature may or may not occur. As such, use of the term “optionally” includes instances where the feature is present, and also instances where the feature is not present. For example, a group “optionally substituted by one hydroxy group” includes groups with and without a hydroxy substituent.

The term “substituted” as used herein means that one or more hydrogens on the designated group are replaced by the indicated substituent(s) provided that any atom(s) bearing a substituent maintains a permitted valency. Substituent combinations encompass only stable compounds and stable synthetic intermediates. The term “stable” when used in this context, means that the relevant compound or intermediate is sufficiently robust to be isolated and have utility either as a synthetic intermediate or as an agent having potential therapeutic utility. If a group is not described as “substituted”, or “optionally substituted”, it is to be regarded as unsubstituted (i.e., none of the hydrogens on the designated group have been replaced).

The term “therapeutically effective amount” refers to an amount of a compound of the invention, which is effective to provide “therapy” in a subject, or to “treat” a disease or disorder in a subject.

The terms “therapy” and “treatment” as used herein refer to dealing with a disease in order to entirely or partially relieve one, some or all of its symptoms, or to correct or compensate for the underlying pathology. The terms “therapy” and “treatment” also include “prophylaxis” unless otherwise indicated. The terms “therapeutic” and “therapeutically” should be interpreted in a corresponding manner. Similarly, the term “treat” can be regarded as “applying therapy”.

The term “prophylaxis” includes primary prophylaxis to prevent the development of the disease and secondary prophylaxis whereby the disease has already developed and the subject is temporarily or permanently protected against exacerbation of the disease or the development of new symptoms associated with the disease.

The term “subject” as used herein with reference to a method of treatment, refers to a warm-blooded animal to whom the treatment is applied. Examples of warm-blooded animals include, but are not limited to, primates, livestock animals (for example, sheep, cows, pigs, goats, horses) and companion animals (for example, cats and dogs). In one embodiment, the warm-blooded animal is a human.

Asymmetric centers may exist in the compounds described herein. The asymmetric centers may be designated as (R) or (S), depending on the configuration of substituents in three-dimensional space at the chiral carbon atom. All stereochemical isomeric forms of the compounds, including diastereomeric, enantiomeric, and epimeric forms, as well as d-isomers and I-isomers, and mixtures thereof, including enantiomerically enriched and diastereomerically enriched mixtures of stereochemical isomers, are within the scope of the invention.

Individual enantiomers can be prepared synthetically from commercially available enantiopure starting materials or by preparing enantiomeric mixtures and resolving the mixture into individual enantiomers. Resolution methods include conversion of the enantiomeric mixture into a mixture of diastereomers and separation of the diastereomers by, for example, recrystallization or chromatography, and any other appropriate methods known in the art. Starting materials of defined stereochemistry may be commercially available or made and, if necessary, resolved by techniques well known in the art.

The compounds described herein may also exist as conformational or geometric isomers, including cis, trans, syn, anti, entgegen (E), and zusammen (Z) isomers. All such isomers and any mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol, imine/enamine, and thioketone/enethiol tautomerism.

The compounds described herein may also exist as isotopologues and isotopomers, wherein one or more atoms in the compounds are replaced with different isotopes. Suitable isotopes include, for example, ¹H, ²H (D), ³H (T), ¹²C, ¹³C, ¹⁴C, ¹⁶O, and ¹⁸O. Procedures for incorporating such isotopes into the compounds described herein will be apparent to those skilled in the art. Isotopologues and isotopomers of the compounds described herein are also within the scope of the invention.

Also within the scope of the invention are salts of the compounds described herein, including pharmaceutically acceptable salts. Such salts include, acid addition salts, base addition salts, and quaternary salts of basic nitrogen-containing groups. Acid addition salts can be prepared by reacting compounds, in free base form, with inorganic or organic acids. Examples of inorganic acids include, but are not limited to, hydrochloric, hydrobromic, nitric, sulfuric, and phosphoric acid. Examples of organic acids include, but are not limited to, acetic, trifluoroacetic, propionic, succinic, glycolic, lactic, malic, tartaric, citric, ascorbic, maleic, fumaric, pyruvic, aspartic, glutamic, stearic, salicylic, methanesulfonic, benzenesulfonic, isethionic, sulfanilic, adipic, butyric, and pivalic. Base addition salts can be prepared by reacting compounds, in free acid form, with inorganic or organic bases. Examples of inorganic base addition salts include alkali metal salts, alkaline earth metal salts, and other physiologically acceptable metal salts, for example, aluminium, calcium, lithium, magnesium, potassium, sodium, or zinc salts. Examples of organic base addition salts include amine salts, for example, salts of trimethylamine, diethylamine, ethanolamine, diethanolamine, and ethylenediamine. Quaternary salts of basic nitrogen-containing groups in the compounds may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like.

The term “pharmaceutically acceptable” is used to specify that an object (for example a salt, dosage form, diluent or carrier) is suitable for administration to a subject, in particular, a human subject. An example list of pharmaceutically acceptable salts can be found in the Handbook of Pharmaceutical Salts: Properties, Selection and Use, P. H. Stahl and C. G. Wermuth, editors, Weinheim/Zurich: Wiley-VCH/VHCA, 2002.

A suitable pharmaceutically acceptable salt of a compound of Formula (I) is, for example, an acid-addition salt. An acid-addition salt of a compound of Formula (I) may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulfuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

The compounds described herein may form or exist as solvates with various solvents. If the solvent is water, the solvate may be referred to as a hydrate, for example, a mono-hydrate, a di-hydrate, or a tri-hydrate. All solvated forms and unsolvated forms of the compounds described herein are within the scope of the invention.

The general chemical terms used herein have their usual meanings. Standard abbreviations for chemical groups are well known in the art and take their usual meaning, eg, Me=methyl, Et=ethyl, iPr=isopropyl, Bu=butyl, t-Bu=tert-butyl, Ph=phenyl, Bn=benzyl, Ac=acetyl, Boc=tert-butoxycarbonyl, Fmoc=9-fluorenylmethoxycarbonyl, Tf=triflate, OMOM=methoxymethyl ether, OMEM=methoxyethoxymethyl ether, OBOM=benzyloxymethyl ether, OTBDMS=tert-butyldimethylsilyl ether, DPPA=diphenylphosphoryl azide, NBS=N-bromosuccinimide, NIS=N-iodosuccinimide, OPMB=4-methoxybenzyl ether, EDCI=1-ethyl-3-(3-dimethylaminopropyl)carbodiimide, HOBt=hydroxybenzotriazole, OSEM=[2-(trimethylsilyl)ethoxy]methyl ether, Alloc=allyloxycarbonyl, Cbz=benzyloxycarbonyl, Teoc=(2-(trimethylsilyl)ethoxycarbonyl, TEMPO=2,2,6,6-tetramethyl-1-piperidinyloxy, Troc=2,2,2-trichlooroethylcarbonyl, and the like.

The terms “halo”, “halide” or “halogen group” used herein refer to a fluoro, chloro, bromo or iodo group.

The term “amino” as used herein refers to —NH₂.

The term “alkyl” as used herein refers to refers to a saturated straight or branched acyclic hydrocarbon group, such as a straight or branched group of 1-20, 1-8, or 1-6 carbon atoms, referred to herein as (C₁-C₂₀)alkyl, (C₁-C₅)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, and the like.

The term “alkenyl” as used herein refers to an unsaturated straight or branched acyclic hydrocarbon group having at least one carbon-carbon double bond, such as a straight or branched group of 2-20, 2-8, or 2-6 carbon atoms, referred to herein as (C₂-C₂₀)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “cycloalkyl” as used herein refers to a saturated hydrocarbon ring group. The prefix “Cₓ-C_{y}”, wherein x and y are each an integer, when used in combination with the term “cycloalkyl” refers to the number of ring carbon atoms in the cycloalkyl group. Examples of cycloalkyl groups include cyclopropyl, cyclobutyl, cyclopentyl, and cyclohexyl as well as bridged and caged saturated ring groups such as, for example, adamantane.

The term “heterocycloalkyl” refers to a single aliphatic ring, containing at least 2 carbon atoms in addition to 1-3 heteroatoms independently selected from oxygen, sulfur, and nitrogen, as well as combinations comprising at least one of the foregoing heteroatoms. The prefix “Cₓ-C_{y}”, wherein x and y are each an integer, when used in combination with the term “heterocycloalkyl” refers to the number of ring carbon atoms in the heterocycloalkyl group. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperdyl, and 2,5-piperzinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

The term “aryl” as used herein refers to a cyclic aromatic hydrocarbon group that does not contain any ring heteroatoms. Aryl groups include monocyclic and bicyclic ring systems. Examples of aryl groups include, but are not limited to, phenyl, azulenyl, heptalenyl, indenyl, indanyl, pentalenyl, and naphthyl. In some embodiments, aryl groups have from 6 to 20, 6 to 14, 6 to 12, or 6 to 10 carbon atoms in the ring(s). In some embodiments, the aryl groups are phenyl or naphthyl. Aryl groups include aromatic-carbocycle fused ring systems. Examples include, but are not limited to, indanyl and tetrahydronaphthyl. The prefix “Cₓ-C_{y}”, wherein x and y are each an integer, when used in combination with the term “aryl” refers to the number of ring carbon atoms in the aryl group. In some embodiments, “aryl” groups may be substituted with one or more optional substituents as described herein.

The term “heteroaryl” as used herein refers to an aromatic ring system containing 5 or more ring atoms, of which, one or more is a heteroatom. In some embodiments, the heteroatom is nitrogen, oxygen, or sulfur. A heteroaryl group is a variety of heterocyclic group that possesses an aromatic electronic structure. In some embodiments, heteroaryl groups include mono-, bi- and tricyclic ring systems having from 5 to 20, 5 to 16, from 5 to 14, from 5 to 12, from 5 to 10, from 5 to 8, or from 5 to 6 ring atoms. Heteroaryl groups include, but are not limited to pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, imidazyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl. The prefix “x-y membered”, wherein x and y are each an integer, when used in combination with the term “heteroaryl” refers to the number of ring atoms in the heteroaryl group. In some embodiments “heteroaryl” groups may be substituted with one or more optional substituents as described herein.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

5.2 DNA-PK Inhibitor Compounds of the Invention

The invention relates to imidazo[4,5-c]pyridine-2-one compounds that inhibit DNA-PK and to selected prodrug versions of these compounds.

In a first aspect, the invention provides a compound of Formula I or salt thereof

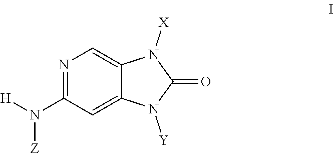

wherein:
X is selected from the group consisting of:
(a) —H,
(b) —(C₁-C₆)alkyl optionally substituted with one or more groups independently selected from —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NRIC(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂ NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl; and
(c) —(C₂-C₆)alkenyl optionally substituted with one or more groups independently selected from —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NRC(O)NR¹R¹, —SH, —SR¹, —S(O)H, —S(O)R¹, —SO₂R′, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl and wherein -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR², and —C(O)NR²R², wherein R² is —(C₁-C₆)alkyl;
Y is selected from the group consisting of:
(a) —(C₁-C₆)alkyl optionally substituted with one or more groups independently selected from —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkyl optionally substituted with —OH, —OR¹, —NH₂, —NHR¹ or —NR¹R¹, and —(C₃-C₇)heterocycloalkyl which contains an oxygen or nitrogen atom in the ring and which is optionally substituted with —OH, —OR¹, —NH₂, —NHR¹, —NR¹R¹, or —(C₁-C₆)alkyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;
(b) —(C₂-C₆)alkenyl optionally substituted with one or more groups independently selected from —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkyl optionally substituted with —OH, —OR¹, —NH₂, —NHR¹ or —NR¹R¹, and —(C₃-C₇)heterocycloalkyl which contains an oxygen or nitrogen atom in the ring and which is optionally substituted with —OH, —OR¹, —NH₂, —NHR¹, —NR¹R¹, or —(C₁-C₆)alkyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;
(c) —(C₃-C₇)cycloalkyl optionally substituted with one or more groups independently selected from —R¹, —OH, -halo, —OR¹, —OC(O)H, OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;
(d) —(C₃-C₇)heterocycloalkyl optionally substituted with one or more groups independently selected from —R¹, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;
(e) —(C₄-C₅)aryl optionally substituted with one or more groups independently selected from —R¹, —OH, -halo, —OR¹, —OC(O)H, OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NHC(O)NR¹R¹, —NR¹C(O)NH₂, —NR¹C(O)NHR¹, —NR¹C(O)NH₂, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —O₂R², —CHO, —C(O)R², —C(O)NH₂, —C(O)NHR² and —C(O)NR²R², wherein R² is —(C₁-C₆)alkyl; and
(f) —(C₅-C₁₂)heteroaryl optionally substituted with one or more groups independently selected from —R¹, —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC (O)NH₂, —NHC(O)NHR¹, —NR¹C(O)NH₂, —NHC(O)NR¹R¹, —NR¹C(O)NHR¹, —NRC(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF 3, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl; and
Z is selected from the group consisting of:
(a) —(C₄-C₅)aryl optionally substituted with one or more groups independently selected from —R¹, —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, morpholinyl, piperazinyl, pyridinyl and pyrimidinyl;
wherein each R¹ is independently selected from —(C₁-C₆)alkyl and —(C₄-C₅)aryl, each of which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein each of morpholinyl, piperazinyl, pyridinyl and
pyrimidinyl are optionally substituted with one or more groups selected from —(C₁-C₆)alkyl, -halo, —OH, —OR², —NO₂, —NH₂, —NHR², —NR²R², —SH, —SR², —SO₂R², —SO₂NH², —CF₃, —CHF₂, CH₂F, —CN, —CO₂H, —CO₂R², —CHO, —C(O)R², —C(O)NH₂, —C(O)NHR² and —C(O)NR²R², wherein R² is —(C₁-C₆)alkyl; (b) —(C₅-C₁₂)heteroaryl optionally substituted with one or more groups independently selected from —R¹, —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, morpholinyl, and piperazinyl;
wherein each R¹ is independently selected from —(C₁-C₆)alkyl and —(C₄-C₈)aryl, each of which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl, and
wherein each of morpholinyl and piperazinyl is optionally substituted with one or more group selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl.

In one embodiment X is
(b) —(C₁-C₆)alkyl optionally substituted with one or more groups independently selected from —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂ NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl; and
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein -Ph is optionally substituted with one or more groups independently
selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl.

In one embodiment X is —(C₁-C₆)alkyl. In one embodiment X is Me.

In one embodiment X is —(C₁-6)alkyl optionally substituted with OH or NH₂.

In one embodiment Y is selected from the group consisting of (c), (d) and (e) as set out above.

In one embodiment Y is selected from the group consisting of —(C₃-C₇)cycloalkyl, oxetanyl, tetrahydrofuranyl, tetrahydropyranyl, cyclohexanyl, pyrrolidinyl and piperidinyl and phenyl, each of which is optionally substituted with one or more groups independently selected from —R¹, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;

In one embodiment Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

In one embodiment Y is selected from the group consisting of tetrahydropyranyl, aminocyclohexanyl, hydroxycyclohexanyl, methoxycyclohexanyl, and piperidinyl.

In one embodiment Y is 4-tetrahydropyranyl or 4-piperidinyl.

In one embodiment Y is selected from the group consisting of furanyl, pyrrolyl and pyridinyl.

In one embodiment Y is selected from the group consisting of 4-methoxycyclohexanyl, 4-hydroxycyclohexanyl, or 4-aminocyclohexanyl.

In one embodiment Y is 4-hydroxyphenyl or 4-methoxyphenyl.

In one embodiment Z is —(C₅-C₁₂)heteroaryl which is selected from the group consisting of furanyl, thiophenyl, pyrrolyl, pyridinyl, imidazolyl, thiazolyl, pyrimidinyl, pyrazinyl, indolyl, isoindolyl, quinolinyl, isoquinolinyl, purinyl, benzodioxolyl, quinoxalinyl, benzothiazinyl, triazolopyridinyl, benzothiazolyl, benzoxazolyl, benzodioxolyl and imidazopyridinyl, each of which may be optionally substituted with one or more groups
independently selected from —R¹, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl and —(C₄-C₅)aryl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;

In one embodiment Z is —(C₅-C₁₂)heteroaryl which is selected from the group consisting of pyrimidinyl, pyrazinyl, indolyl, isoindolyl, quinolinyl, isoquinolinyl, purinyl, benzodioxolyl, quinoxalinyl, benzothiazinyl, triazolopyridinyl, benzothiazolyl, benzoxazolyl, benzodioxolyl and imidazopyridinyl, each of which may be optionally substituted with one or more groups selected from —(C₁-C₆)alkyl, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NHC(O)NR¹R¹, —NRC(O)NHR¹, —NRC(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl and —(C₄-C₅)aryl, each of which is optionally substituted with halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR², —C(O)NR²R² wherein R² is —(C₁-C₆)alkyl.

In one embodiment, Z is —(C₅-C₁₂)heteroaryl substituted with (C₁-C₆)alkyl, preferably Me.

In one embodiment, Z is —(C₄-C₅)aryl substituted with (C₁-C₆)alkyl, preferably Me.

In one embodiment Z is phenyl optionally substituted with one or more of R¹, —OH, —OR¹, -halo, —NO₂, —NH₂, —NHR¹, —NR¹R¹, —SO₂R¹ and -Bn wherein —R¹ is (C₁-C₆)alkyl, preferably Me.

In one embodiment Z is phenyl substituted at the 4-position with any one of —OMe, —Cl and —OH.

In one embodiment Z is phenyl substituted at the 5-position with one of —SO₂R¹ and —NO₂ wherein —R¹ is (C₁-C₆)alkyl, preferably Me.

In one embodiment Z is selected from the group consisting of 4-methoxy-2-methylphenyl, 4-chloro-2-methylphenyl, 5-(methylsulfonyl)-2-methylphenyl and 4-hydroxy-2-methylphenyl.

In one embodiment Z is 4-methoxy-2-methylphenyl.

In a second aspect, the invention provides a compound of Formula II or salt thereof

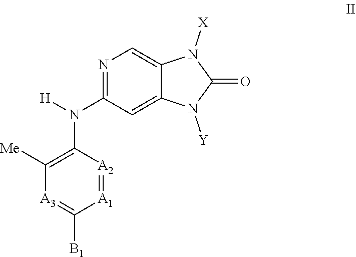

wherein X and Y are as defined for Formula I,
A₁, A₂ and A₃ are independently selected from CH or N, and
B₁ is selected from the group consisting of —OH, —OR¹, halo, —NO₂, —NH₂, NHR¹, —SO₂R¹ and —OBn,
wherein R¹ is —(C₁-C₆)alkyl optionally substituted with halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl.

In one embodiment X is Me.

In one embodiment Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

In one embodiment Y is selected from the group consisting of tetrahydropyranyl, aminocyclohexanyl, hydroxycyclohexanyl, methoxycyclohexanyl, and piperidinyl.

In one embodiment Y is 4-tetrahydropyranyl or 4-piperidinyl.

In one embodiment Y is selected from the group consisting of furanyl, pyrrolyl and pyridinyl.

In one embodiment Y is selected from the group consisting of 4-methoxycyclohexanyl, 4-hydroxycyclohexanyl, or 4-aminocyclohexanyl.

In one embodiment Y is 4-hydroxyphenyl or 4-methoxyphenyl.

In one embodiment A₁ is N, A₂ and A₃ are C and B₁ is OMe.

In a third aspect, the invention provides a compound of Formula III or salt thereof

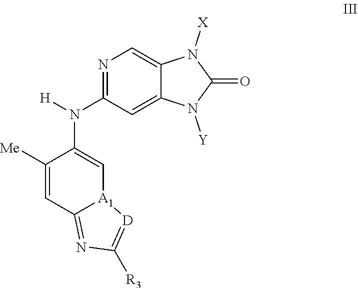

wherein X and Y are as defined for Formula I,
A₁ is N or C,
D is selected from the group consisting of N, O, S, and
R³ is selected from the group consisting of H, —(C₁-C₆)alkyl, —CO₂R¹, —CONHR¹ and CONHR¹R¹,
wherein R¹ is —(C₁-C₆)alkyl.

In one embodiment X is Me.

In one embodiment Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

In one embodiment Y is selected from the group consisting of tetrahydropyranyl, aminocyclohexanyl, hydroxycyclohexanyl, methoxycyclohexanyl, and piperidinyl.

In one embodiment Y is 4-tetrahydropyranyl or 4-piperidinyl.

In one embodiment Y is selected from the group consisting of furanyl, pyrrolyl and pyridinyl.

In one embodiment Y is selected from the group consisting of 4-methoxycyclohexanyl, 4-hydroxycyclohexanyl, or 4-aminocyclohexanyl.

In one embodiment Y is 4-hydroxyphenyl or 4-methoxyphenyl.

In one embodiment R³ is H, Me, OMe, or CO₂Me.

In one embodiment A₁ is N, D is CH and R³ is H.

In one embodiment A₁ is N, D is N and R³ is H.

In one embodiment A₁ is N, D is CH and R³ is CO₂Me.

In a fourth aspect, the invention provides a compound of Formula IV

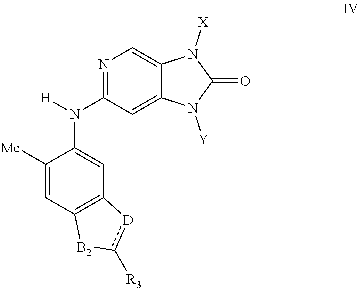

wherein X and Y are as defined for Formula I,
B₂ and D are independently selected from the group consisting of N, O and S, [symbol] depicts a single or double bond, wherein [symbol] is a single bond unless D is N, and R³ is selected from the group consisting of H, —(C₁-C₆)alkyl, —CO₂R¹, —CONHR¹ and CONHR¹R¹, wherein R¹ is —(C₁-C₆)alkyl.

In one embodiment X is Me.

In one embodiment Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

In one embodiment Y is selected from the group consisting of tetrahydropyranyl, aminocyclohexanyl, hydroxycyclohexanyl, methoxycyclohexanyl, and piperidinyl.

In one embodiment Y is 4-tetrahydropyranyl or 4-piperidinyl.

In one embodiment Y is selected from the group consisting of furanyl, pyrrolyl and pyridinyl.

In one embodiment Y is selected from the group consisting of 4-methoxycyclohexanyl, 4-hydroxycyclohexanyl, or 4-aminocyclohexanyl.

In one embodiment Y is 4-hydroxyphenyl or 4-methoxyphenyl.

In one embodiment R³ is H, Me or OMe.

In one embodiment B₂ is N and D is O or S and R³ is Me.

In one embodiment B₂ is N and D is O.

In addition to the DNA-PK inhibitors set out above, the invention also includes prodrug compounds comprising a DNA-PK inhibitor of the invention and an aromatic nitroheterocycle or nitrocarbocycle that fragments when reduced (a reductive prodrug trigger).

Accordingly, in a fifth aspect the invention provides a compound of Formula V or salt thereof

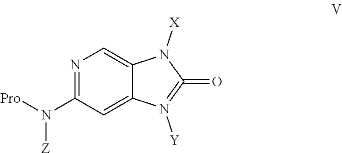

wherein X, Y and Z are as defined for Formula I and
Pro is selected from the group consisting of:

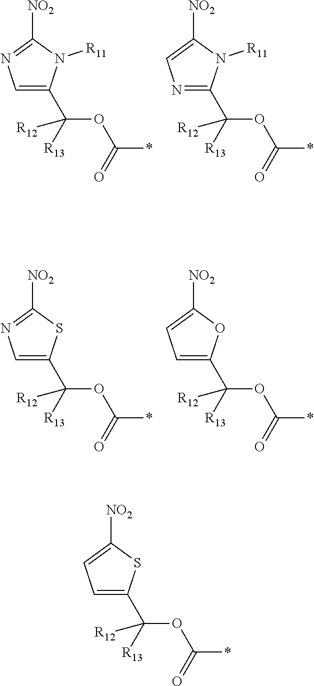

wherein * indicates the point of attachment to the N atom of Formula V;
wherein R₁₁ is —(C₁-C₆)alkyl optionally substituted with —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² and —C(O)NR²R² wherein R² is —(C₁-C₆)alkyl and
wherein -Ph is optionally substituted with one or more —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR², —C(O)NR²R², wherein R² is —(C₁-C₆)alkyl; and R₁₂ and R₁₃ are independently selected from the group consisting of —H, -Me and -Et.

In one embodiment X is Me.

In one embodiment Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

In one embodiment Y is selected from the group consisting of tetrahydropyranyl, aminocyclohexanyl, hydroxycyclohexanyl, methoxycyclohexanyl, and piperidinyl.

In one embodiment Y is 4-tetrahydropyranyl or 4-piperidinyl.

In one embodiment Y is selected from the group consisting of furanyl, pyrrolyl and pyridinyl.

In one embodiment Y is selected from the group consisting of 4-methoxycyclohexanyl, 4-hydroxycyclohexanyl, or 4-aminocyclohexanyl.

In one embodiment Y is 4-hydroxyphenyl or 4-methoxyphenyl.

In one embodiment, Z is —(C₅-C₁₂)heteroaryl substituted with (C₁-C₆)alkyl, preferably Me.

In one embodiment Z is

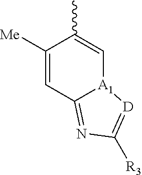

wherein A₁ is N or C, D is selected from the group consisting of N, O, S, and R³ is selected from the group consisting of H, —(C₁-C₆)alkyl, —CO₂R¹, —CONHR¹ and CONHR¹R¹, wherein R¹ is —(C₁-C₆)alkyl.

In one embodiment R³ is H, Me, OMe, or CO₂Me.

In one embodiment A₁ is N, D is CH and R³ is H.

In one embodiment A₁ is N, D is N and R³ is H.

In one embodiment A₁ is N, D is CH and R³ is CO₂Me.

In one embodiment Z is

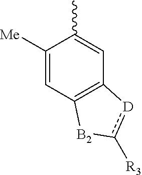

wherein B₂ D are independently selected from the group consisting of N, O and S, [symbol] depicts a single or double bond, wherein [symbol] is a single bond unless D is N, and R³ is selected from the group consisting of H, —(C₁-C₆)alkyl, —CO₂R¹, —CONHR¹ and CONHR¹R¹, wherein R¹ is —(C₁-C₆)alkyl.

In one embodiment R³ is H, Me or OMe.

In one embodiment B₂ is N and D is O or S and R³ is Me.

In one embodiment B₂ is N and D is O.

In one embodiment, Z is —(C₄-C₅)aryl substituted with (C₁-C₆)alkyl, preferably Me.

In one embodiment Z is phenyl optionally substituted with one or more of R¹, —OH, —OR¹, -halo, —NO₂, —NH₂, —NHR¹, —NR¹R¹, —SO₂R¹ and -Bn wherein —R¹ is (C₁-C₆)alkyl, preferably Me.

In one embodiment Z is phenyl substituted at the 4-position with any one of —OMe, —C₁ and —OH.

In one embodiment Z is phenyl substituted at the 5-position with one of —SO₂R¹ and —NO₂ wherein —R¹ is (C₁-C₆)alkyl, preferably Me.

In one embodiment Z is selected from the group consisting of 4-methoxy-2-methylphenyl, 4-chloro-2-methylphenyl, 5-(methylsulfonyl)-2-methylphenyl and 4-hydroxy-2-methylphenyl.

In one embodiment Z is 4-methoxy-2-methylphenyl.

In one embodiment, Pro is

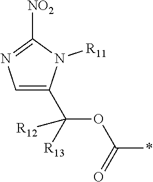

wherein R₁₂ and R₁₃ are defined as above.

In one embodiment, Pro is selected from the group consisting of

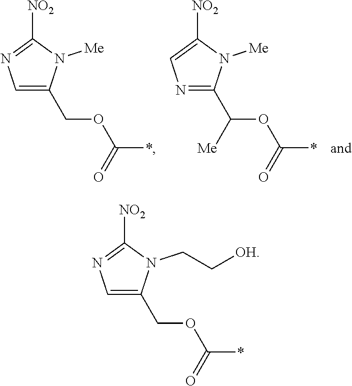

In one embodiment X is Me, Y is tetrahydropyranyl, Z is 4-methoxy-2-methylphenyl and Pro is

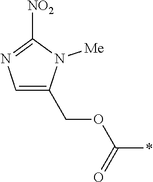 [SMILES: *C(=O)OCc1cnc([N+](=O)[O-])n1C]

In a sixth aspect the invention provides a compound of Formula VI or salt thereof

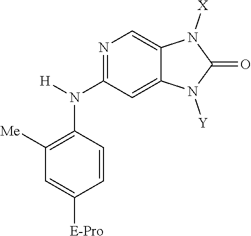

wherein X and Y are as defined for Formula I,
E is selected from —O—, —NHCO₂—, —N(Me)CO₂—, —COO—, —NH(C₁-C₆)alkyl, —O—(C₁-C₆)alkyl-N-dimethylamino-, —NH(C₁-C₆)alkyl-N-dimethylamino-, —NHCO(C₁-C₆)alkyl-N-dimethylamino- and —NHCOCH═CHCH₂—N-dimethylamino-, and Pro is selected from the group consisting of:

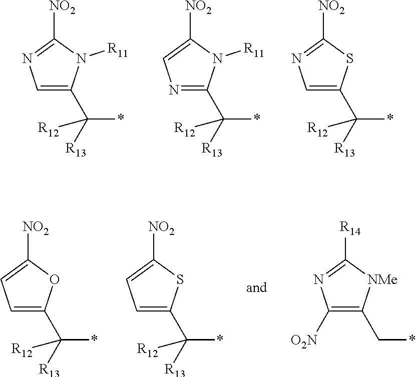

wherein * indicates the point of attachment to E of Formula VI;
wherein R₁₁ is —(C₁-C₆)alkyl optionally substituted with —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —C₁₋₆ alkyl, and
wherein -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl, and
R₁₂ and R₁₃ are independently selected from the group consisting of —H, -Me and -Et; and R₁₄ is selected from the group comprising —H, -Me, -Et, —OMe, —CF₃, —CN and ethynyl;
with the proviso that Pro is

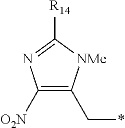

when E is selected from —O—(C₁-C₆)alkyl-N-dimethylamino-, —NH(C₁-C₆)alkyl-N-dimethylamino-, —NHCO(C₁-C₆)alkyl-N-dimethylamino- or —NHCOCH═CHCH₂—N-dimethylamino-.

In one embodiment, Pro is

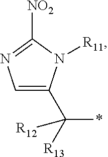

wherein R₁₂ and R₁₃ are as defined above and E is —O—.

In one embodiment, Pro is selected from the group consisting of

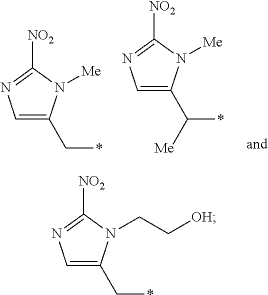

### and E is —O— or NHCO₂.

In one embodiment, Pro is

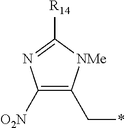

wherein R₁₄ is defined as above and E is O—(C₁-C₆)alkyl-N-dimethylamino, preferably —OCH₂CH₂NMe₂ or —OCH₂CH₂CH₂NMe₂.

In one embodiment X is Me.

In one embodiment Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

In one embodiment Y is selected from the group consisting of tetrahydropyranyl, aminocyclohexanyl, hydroxycyclohexanyl, methoxycyclohexanyl, and piperidinyl.

In one embodiment Y is 4-tetrahydropyranyl or 4-piperidinyl.

In one embodiment Y is selected from the group consisting of furanyl, pyrrolyl and pyridinyl.

In one embodiment Y is selected from the group consisting of 4-methoxycyclohexanyl, 4-hydroxycyclohexanyl, or 4-aminocyclohexanyl.

In one embodiment Y is 4-hydroxyphenyl or 4-methoxyphenyl.

In one embodiment, Pro is selected from the group consisting of

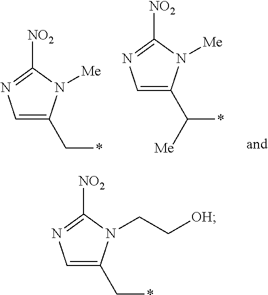

### and E is O

In one embodiment X is Me, Y is tetrahydropyranyl, and Pro is

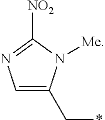 [SMILES: *Cc1cnc([N+](=O)[O-])n1C]

In one embodiment X is Me, Y is 4-methoxycyclohexanyl, and Pro

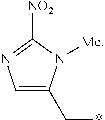 [SMILES: *Cc1cnc([N+](=O)[O-])n1C]

In one embodiment X is Me, Y is 4-methoxyphenyl, and Pro is

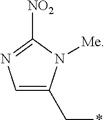 [SMILES: *Cc1cnc([N+](=O)[O-])n1C]

In a seventh aspect the invention provides a compound of Formula VII

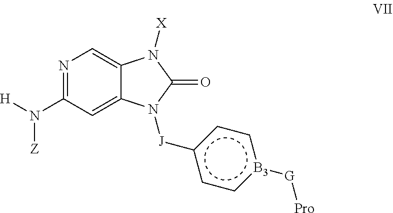

wherein X and Z are as defined for Formula I;
wherein J is CH₂ or absent,

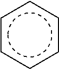

is a saturated or unsaturated ring,
B₃ is C or N,
G is selected from the group consisting of
—O—, —NHCO₂—, —N(Me)CO₂—, —COO—, —NH(C₁-C₆)alkyl, —O—(C₁-C₆)alkyl-N-dimethylamino-, —NH(C₁-C₆)alkyl-N-dimethylamino-, —NHCO(C₁-C₆)alkyl-N-dimethylamino, and —NHCOCH═CHCH₂—N-dimethylamino;
and
Pro is selected from the group consisting of:

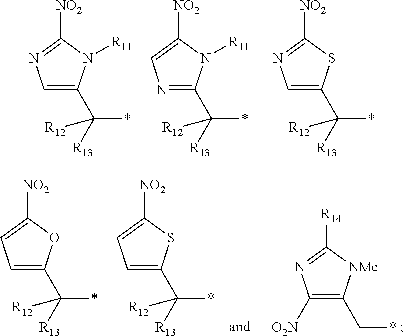

wherein * indicates the point of attachment to G of Formula VII;
wherein R₁₁ is —(C₁-C₆)alkyl optionally substituted with —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein each R² is —C₁-6 alkyl and -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein each R² is —C₁-6 alkyl.
R₁₂ and R₁₃ are independently selected from the group consisting of —H, -Me and -Et; and
R₁₄ is selected from the group comprising —H, -Me, -Et, —OMe, —CF₃, —CN and ethynyl;
with the proviso that Pro is

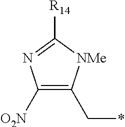

when G is selected from —O—(C₁-C₆)alkyl-N-dimethylamino-, —NH(C₁-C₆)alkyl-N-dimethylamino-,
—NHCO(C₁-C₆)alkyl-N-dimethylamino-, or —NHCOCH═CHCH₂—N-dimethylamino
and the proviso that B₃ is N only where

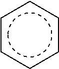

is a saturated ring and
G is —COO— only where B₃ is N; and G is —O— only where B₃ is C.

In one embodiment, Z is —(C₅-C₁₂)heteroaryl substituted with (C₁-C₆)alkyl, preferably Me.

In one embodiment, Z is —(C₄-C₅)aryl substituted with (C₁-C₆)alkyl, preferably Me.

In one embodiment Z is phenyl optionally substituted with one or more of R¹, —OH, —OR¹, -halo, —NO₂, —NH₂, —NHR¹, —NR¹R¹, —SO₂R¹ and -Bn wherein —R¹ is (C₁-C₆)alkyl, preferably Me.

In one embodiment Z is phenyl substituted at the 4-position with any one of —OMe, —C₁ and —OH.

In one embodiment Z is phenyl substituted at the 5-position with one of —SO₂R¹ and —NO₂ wherein —R¹ is (C₁-C₆)alkyl, preferably Me.

In one embodiment Z is selected from the group consisting of 4-methoxy-2-methylphenyl, 4-chloro-2-methylphenyl, 5-(methylsulfonyl)-2-methylphenyl and 4-hydroxy-2-methylphenyl.

In one embodiment Z is 4-methoxy-2-methylphenyl.

In one embodiment, Pro is

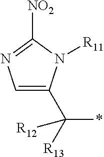

wherein R₁₂ and R₁₃ are defined as above and G is —NHCO₂—.

In one embodiment, Pro is

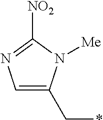 [SMILES: *Cc1cnc([N+](=O)[O-])n1C]

### and G is —NHCO₂—.

In one embodiment, J is absent, the ring is saturated and B₃ is N and G is CO₂ and Pro is NO₂

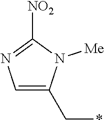 [SMILES: *Cc1cnc([N+](=O)[O-])n1C]

In one embodiment, J is absent, the ring is saturated, B₃ is CH and G is NHCO₂ and Pro is

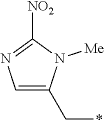 [SMILES: *Cc1cnc([N+](=O)[O-])n1C]

The prodrug compounds of Formulae V-VII comprise a DNA-PK inhibitor of the invention and a reductive “trigger”. The reductive trigger is an aromatic nitroheterocycle or nitrocarbocycle that undergoes fragmentation upon reduction. This nitroheterocyclic or nitrocarbocyclic unit is preferably linked to the DNA-PK inhibitor effector via a carbamate linker, an ether linker or by a quaternary ammonium linker.

The prodrug compounds of the invention are reduced in vivo by enzymes, radiation-induced radicals and/or chemical reducing agents. Fragmentation of the trigger under reductive conditions releases the active DNA-PK inhibitor, with the oxygen or nitrogen atoms to which the trigger was linked remaining part of the released DNA-PK inhibitor.

The prodrug compounds of the invention selectively release DNA-PK inhibitors in tumours; more specifically in regions of hypoxia within tumours. A common feature of most tumours are areas of tumour tissue with low levels of oxygen (hypoxia). “Hypoxia” and related terms such as “hypoxic” refer to a concentration of oxygen in tissue that is significantly lower than the normal physiological concentration of oxygen in healthy well perfused tissue, in particular oxygen tensions below approximately 1% (10,000 parts per million oxygen; 7.6 mmHg). The terms “anoxia” and “anoxic conditions” refer to an absence or near absence of oxygen.

Under hypoxic or anoxic conditions, endogenous one electron enzymes such as cytochrome P450 oxidoreductase (POR) reduce the nitro group to a nitro radical anion.

This process is shown in Scheme 1, with respect to a compound of Formula V.

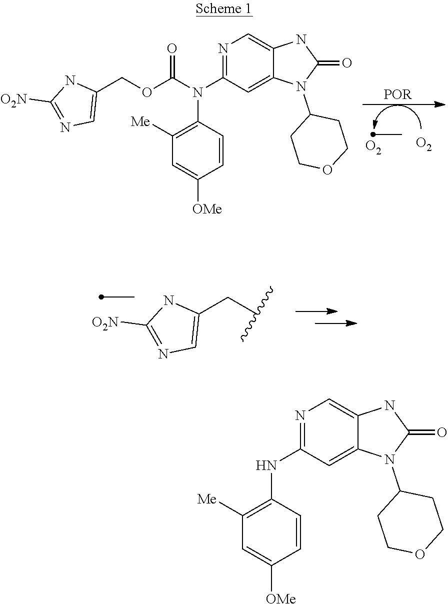

The nitro radical anion acts as an oxygen sensor as it can be reoxidised back to the starting prodrug with concomitant formation of superoxide.

This reduction by one-electron reductases effectively targets the release of the DNA-PK inhibitors to regions of hypoxia within tumours. Reduction is suppressed in normal oxic tissues by the presence of oxygen.

Without wishing to be bound by theory, restriction of DNA-PK inhibitor release to hypoxic tissue and subsequent diffusion of the inhibitor to oxygenated areas of the tumour is believed to be a primary basis for tumour selectivity via endogenous enzymes. This targeting of the release of the DNA-PK inhibitor to tumours is also beneficial in broadening the therapeutic opportunity for such inhibitors.

Overall, the prodrugs of the invention formed by the combination of the fragmenting reductively-activated trigger and a DNA-PK inhibitor have been determined by the applicants to have a number of surprising properties that make them particularly suitable as targeted anti-cancer agents. Foremost amongst these properties is their targeted efficacy. Numerous reductive triggers are already generally known.

However, the combination of each trigger with any particular effector is not guaranteed to be effective and each combination needs to be optimised empirically. The inventors have demonstrated that the particular triggers defined above in combination with the specific DNA-PK inhibitors deactivate the effector, are stable and allow delivery of the prodrug to the tumour. The prodrugs also efficiently fragment under low oxygen conditions to release the cytotoxic effector to have a therapeutic anti-tumour effect.

In an eighth aspect the invention provides a compound of Formula XII

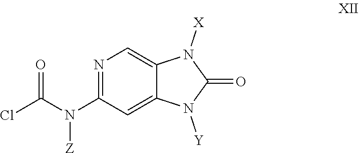

wherein X, Y and Z are as defined for Formula I.

The anilinoimidazopyridinone class, exemplified by the compounds of Formulae I, II, III and IV, provides an opportunity to prepare hypoxia-activated prodrugs of these compounds which is not provided by analogous 2-anilino-7,9-dihydropurin-8-one compounds described by Formula XIII. Preparation of carbamate prodrugs of these DNA-PK inhibitors (e.g. 247, 248, 250, 251, 254-259) is made practical because of the surprising stability of the intermediate carbamoyl chloride (e.g. 249, Scheme 33). Advantages provided by this stable intermediate include isolation and purification which enables improved synthesis conditions and ready purification from the starting material before subsequent reaction and installation of the nitroaryl trigger. Attempts to replicate this procedure using the corresponding 2-anilino-7,9-dihydropurin-8-one core of Formula XIII (such as AZD7648) were fruitless indicating a less stable carbamoyl chloride intermediate.

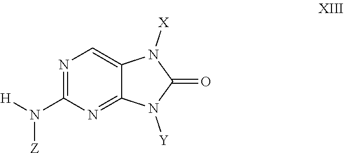

5.3 DNA-PK Inhibition by the Compounds of the Invention

5.3.1 Inhibition of DNA-PKcs and related kinases. The compounds of the invention were evaluated as inhibitors of DNA-PK mediated phosphorylation of a peptide substrate (Table 12). The compounds were also evaluated against the related PI3-K and PIKK member mTOR kinases. The compounds inhibited DNA-PK in the nM to μM range and demonstrated selectivity for DNA-PK compared to PI3K and mTOR.

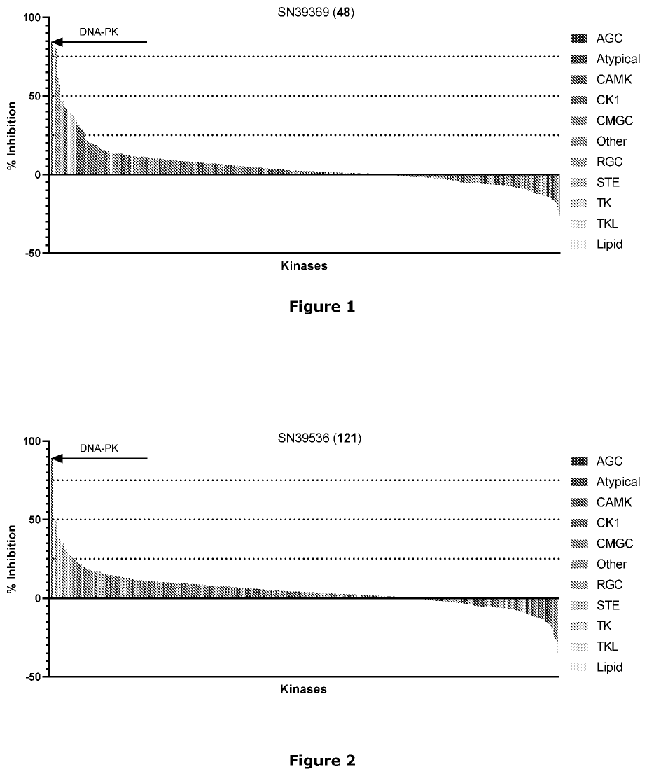

5.3.2 Comparative inhibition of 397 kinases and 20 lipid kinases. The selectivity of particular examples of the invention was evaluated against 397 kinase and 20 lipid kinases at a concentration of 1 μM. Compound 48 (Table 13, FIG. 1) and 121 (Table 14, FIG. 2) demonstrated clear selectivity for DNA-PK compared to other kinases.

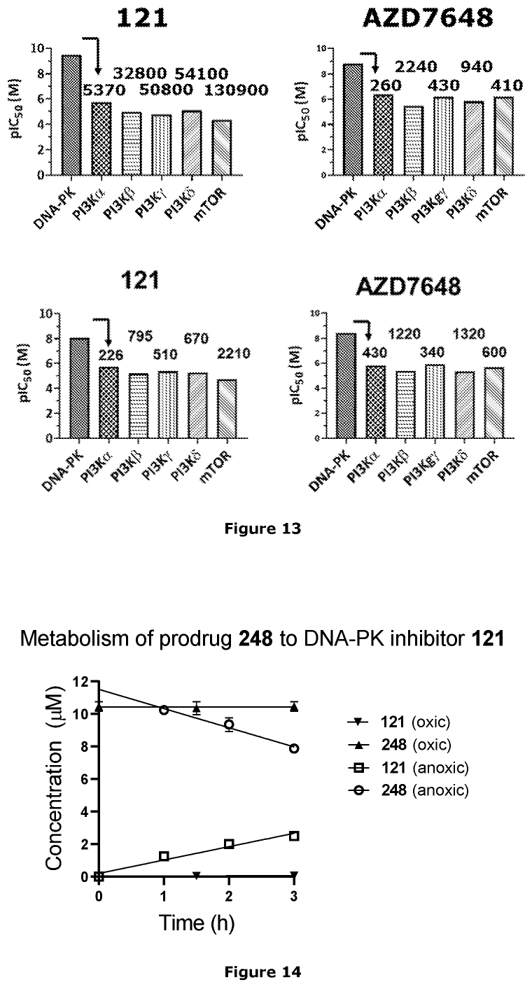

5.3.3. Selectivity for DNA-PKcs compared to other PIKK kinases. The selectivity of the compounds of the invention for DNA-PK compared to other members of the phosphatidylinositol 3-kinase-related kinase family (ATM, ATR, mTOR) and related phosphatidylinositol 3-kinase isoforms (PI3Kα,β, γ, δ) is demonstrated in Table 15 and FIG. 13. Examples of the invention display increased selectivity for DNA-PK over PIKK kinases compared to other known kinase inhibitors (AZD7648).

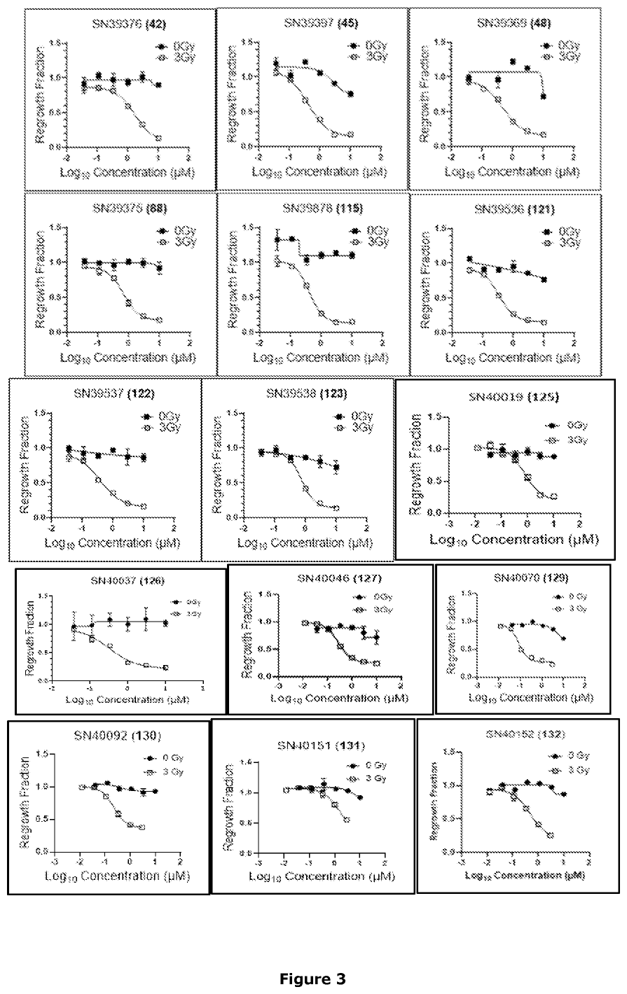

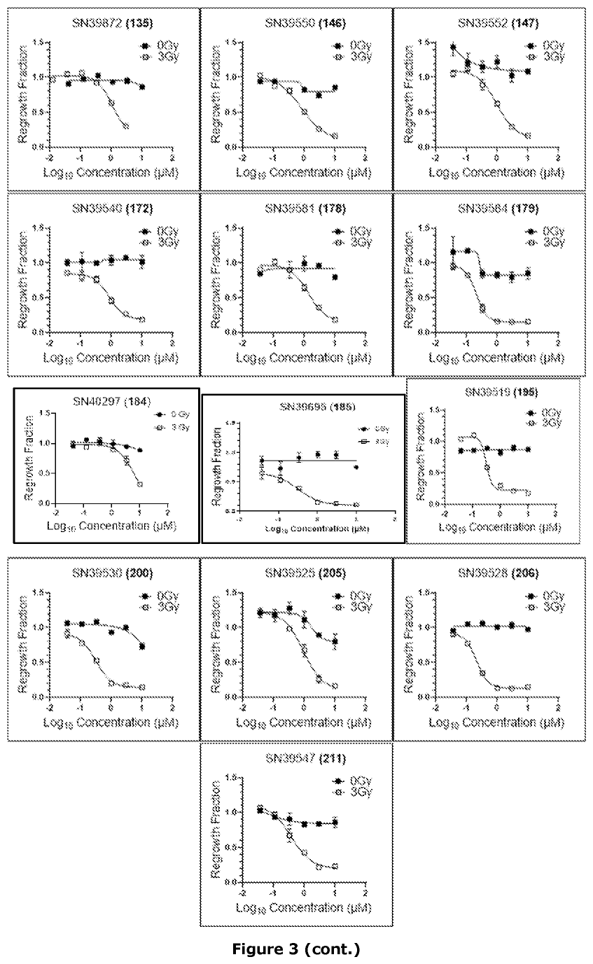

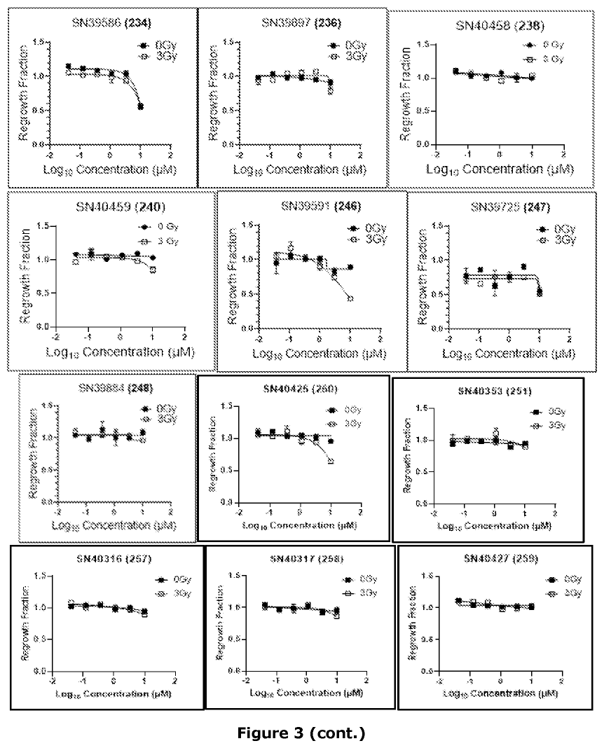

5.3.4 Radiosensitisation of human head and neck cancer cells. The ability of the compounds of the invention to radiosensitise human tumour cells was evaluated under oxic conditions using a proliferative endpoint. UT-SCC-54C cells were cultured with a range of concentrations of compound for one hour before treatment with 0 or 3 Gy of radiation and further incubation for 24 hours. The drug was washed out and the cells allowed to regrow for 5 days before being fixed and stained with sulforhodamine B. Compounds of the invention displayed concentration-dependent radiosensitisation of UT-SCC-54C cells with little cytotoxicity in the absence of radiation (FIG. 3). The cytotoxicity is defined as the drug concentration required for 50% inhibition of regrowth of cultures in the assay: the IC50 value. The radiosensitisation is defined as the drug concentration, in combination with 3 Gy radiation, required for 50% inhibition of regrowth of cultures in the assay: the S50 value (Table 16). Examples of prodrugs of the compounds (e.g., 234, 236, 238, 240, 246, 247, 248, 250, 251, 257, 258, and 259) did not display any differential growth inhibition, demonstrating effective deactivation of the drug.

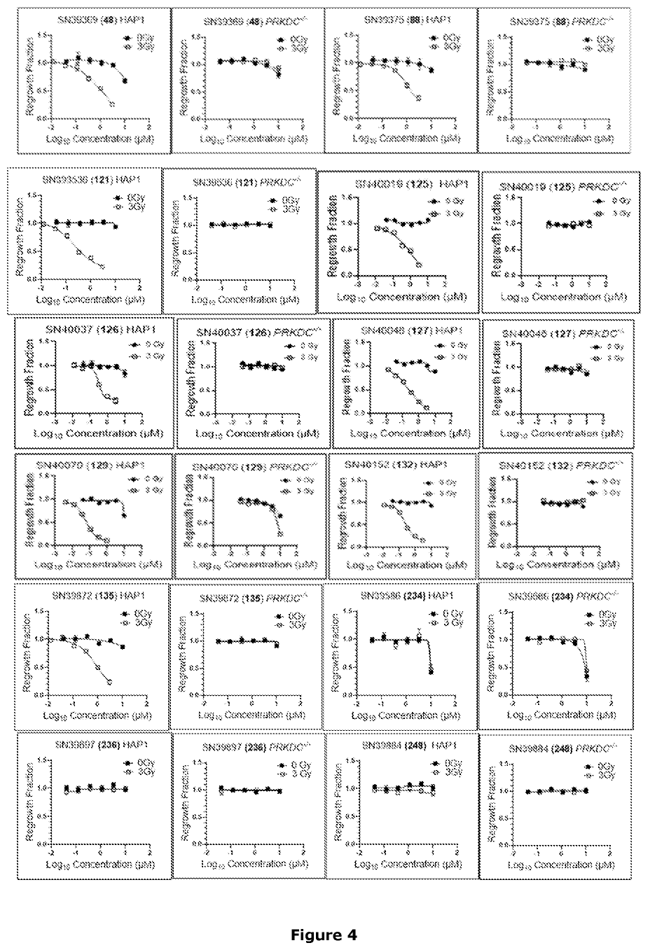

5.3.5. DNA-PKcs dependent radiosensitisation of cells. Examples of the invention were evaluated as radiosensitisers in growth inhibition assay using a HAP1 wild type cell line and DNA-PK null HAP1 line with a CRISPR-induced frameshifting mutation in PRKDC (HAP1/PRKDC^{-/-}). As shown in FIG. 4 and Table 18, compounds 48, 88, 121, 125, 126, 127, 129, 132 and 135 induced concentration-dependent radiosensitisation of HAP1 cells, with clear inhibition of regrowth of cultures after 3 Gy cobalt-60 gamma irradiation, relative to radiation-only, with little effect in unirradiated HAP1 cells. In contrast, compounds 48, 88, 121, 125, 126, 127, 129, 132 and 135 did not radiosensitise the DNA-PK null HAP1 line demonstrating that radiosensitisation of HAP1 cells is dependent on DNA-PK. Furthermore, prodrugs 135, 234, 236 and 248 did not demonstrate any differential growth inhibition in the presence of radiation in either HAP1 cells or PRKDC^{-/-} cells, indicating the deactivation of the drug.

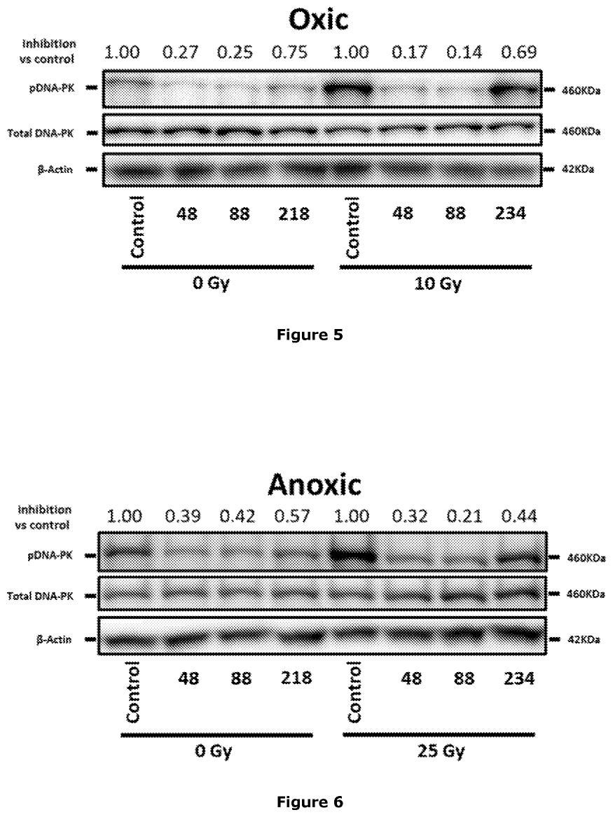

5.3.6 Inhibition of autophosphorylation of Ser2056 of DNA-PKcs in cells. Further evidence of the cellular mechanism of action of the compounds of the invention was demonstrated by inhibition of autophosphorylation of Ser2056 on DNA-PKcs. Irradiation of UT-SCC-54C oxic cells with 10 Gy induced autophosphorylation of Ser 2056 on DNA-PKcs and compounds 48 and 88 demonstrated inhibition of Ser2056 autophosphorylation under oxia (FIG. 5). Similarly, under anoxia compounds 48 and 88 demonstrated inhibition of radiation-induced Ser2056 autophosphorylation (FIG. 6). The prodrug 234 provided little inhibition of Ser2056 autophosphorylation under oxia, but when the experiment was conducted under anoxia, prodrug 234 was able to inhibit phosphorylation of Ser2056 (FIG. 6).

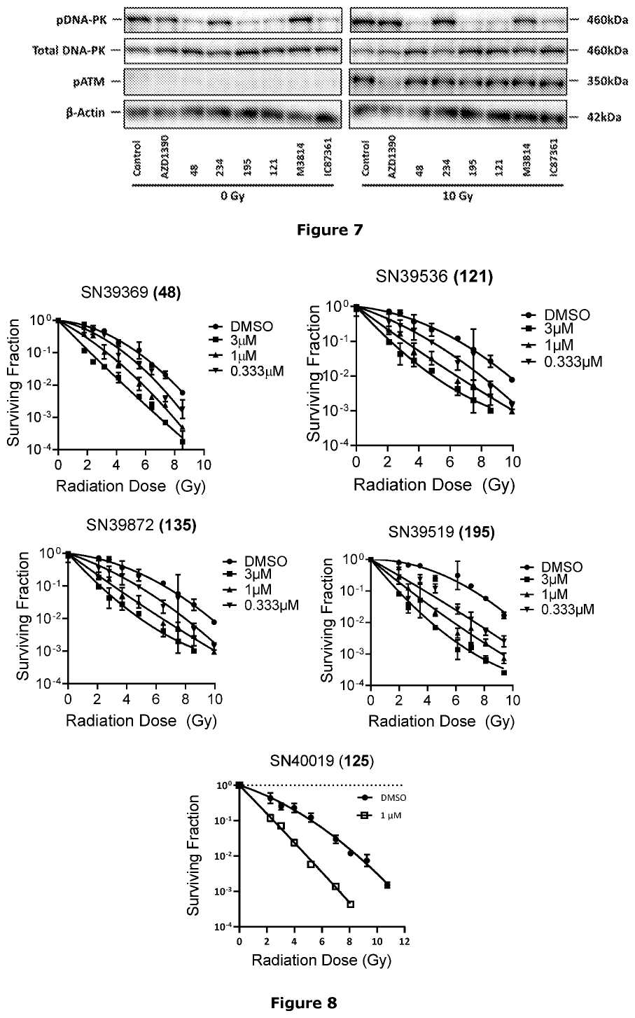

Irradiation of UT-SCC-54C cells under oxia with 10 Gy induced autophosphorylation of Ser 2056 on DNA-PKcs and phosphorylation of Ser1981 on ATM (FIG. 7). Compounds 48, 121, 195, and the published DNA-PK inhibitors M3814 and IC87361, demonstrated inhibition of Ser2056 on DNA-PKcs, but not of Ser1981 on ATM. In contrast, the ATM inhibitor AZD1393 inhibited radiation induced phosphorylation of Ser1981 on ATM but did not affect autophosphorylation of Ser2056 on DNA-PKcs. The prodrug 234 did not inhibit phosphorylation on either enzyme under oxia.

5.3.7. Hypoxia-selective metabolism of prodrugs releases DNA-PK inhibitors. UT-SCC-54C cells selectively metabolised prodrug 248 by under anoxic conditions, but not under oxic conditions, demonstrating hypoxia-selective release of the DNA-PK inhibitor 121 (FIG. 13).

5.3.8 Radiosensitisation of human head and neck cancer cells. Compounds of the invention provided radiosensitisation of human head and neck squamous cell carcinoma cells when evaluated using a clonogenic survival endpoint. For example, compounds 48, 121, 135 and 195 displayed concentration-dependent increases in radiosensitisation (FIG. 8 and Table 18).

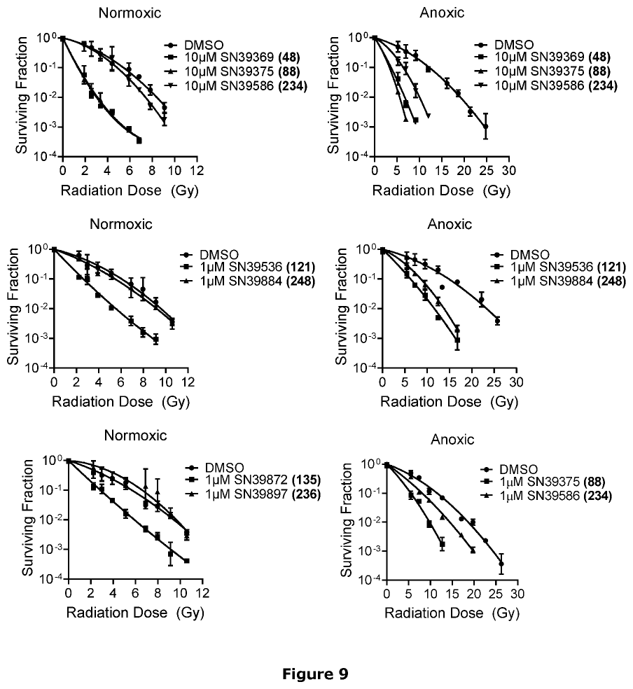

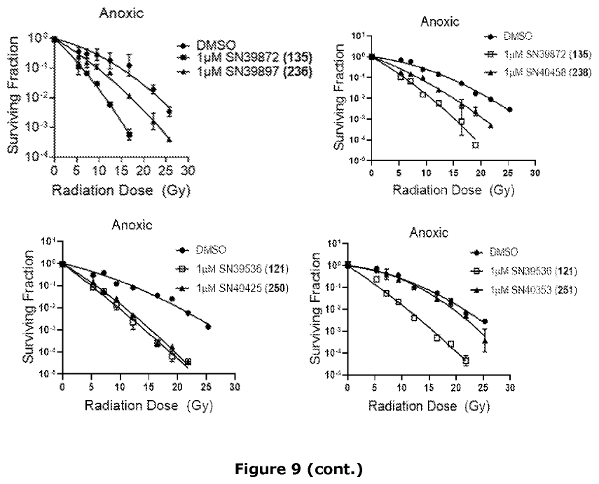

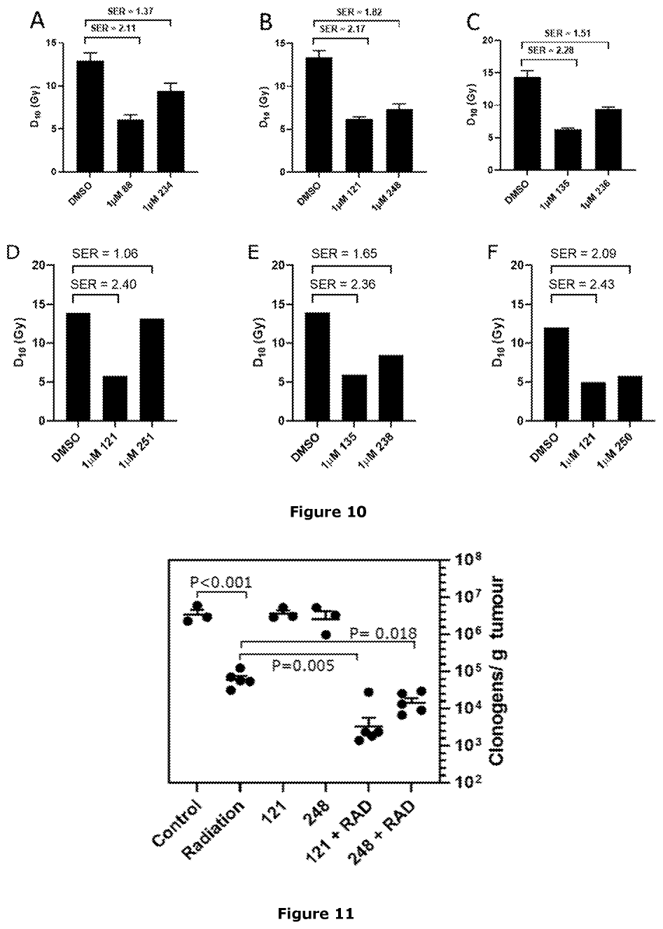

Compound 88 provides radiosensitisation of UT-SCC-54C cells under oxic conditions whereas the prodrug 234 does not (FIG. 9 and Table 18). In contrast, under anoxic conditions the prodrug 234 is activated to provide radiosensitisation (SER=1.37, FIG. 10). Similarly, compound 121 provides radiosensitisation of UT-SCC-54C cells under oxic conditions whereas the prodrug 248 does not. Under anoxic conditions the prodrug 248 is activated to provide radiosensitisation (SER=1.82, FIG. 10). Similarly, compound 236, a prodrug of compound 135, provides sensitisation of UT-SCC-54C cells selectively under anoxia (SER 1.51, FIG. 10). Similarly, compounds 236, 238, 250 and 251, prodrugs of compounds 88, 135, 121 and 122, respectively, provide sensitisation of UT-SCC-54C cells selectively under anoxia (FIGS. 9 and 10).

5.3.9. Radiosensitisation of UT-SCC-54C HNSCC tumours. When administered to mice bearing UT-SCC-54C tumour xenografts, compounds 121 and 248 alone produced no reduction in the clonogens/gram of tumour tissue compared to DMSO control (FIG. 11). Radiation alone (13 Gy) produced ca. 1.5 log reduction in clonogens/gram. Administration of compound 121 in combination with radiation (13 Gy) produced a significant (p=0.005) additional reduction in clonogens/gram of tumour compared to radiation alone. The prodrug 248 produced a smaller, but significant (p=0.018) effect combined with radiation compared to radiation alone. These studies demonstrate the efficacy of DNA-PK inhibitors, and their prodrugs, in combination with radiotherapy in a tumour xenograft model of human head and neck cancer.

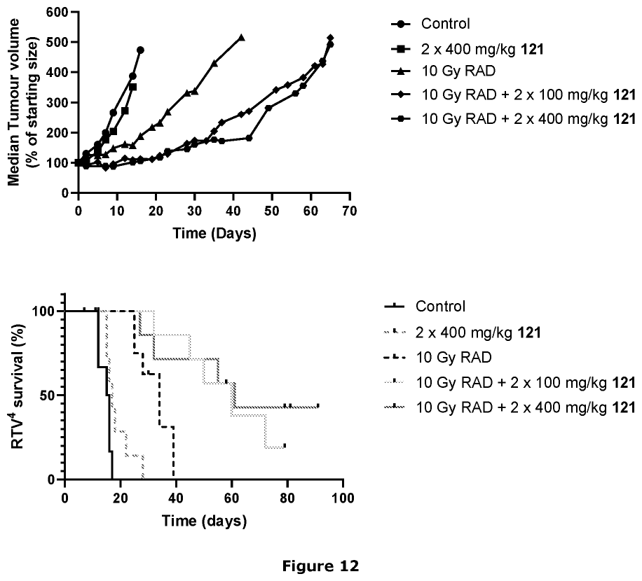

5.3.10. Inhibition of UT-SCC-54C HNSCC tumour growth. When administered to mice bearing UT-SCC-54C tumour xenografts, compound 121 produced little tumour growth inihibition compared to vehicle alone (FIG. 12A). A single dose of radiation (10 Gy) provided modest tumour growth delay while administration of compound 121 in combination with radiation provided considerable additional tumour growth inhibition (FIG. 12B). These studies further demonstrate the efficacy of DNA-PK inhibitors in combination with radiotherapy in a tumour xenograft model of human head and neck cancer.

As described above, the compounds of Formulae I, II, III, IV, V, VI and VII demonstrate selectivity for DNA-PK. In one aspect the invention provides a compound of Formula I, II, III, IV, V, VI and VII that has an IC₅₀ value against DNA-PK of less than 500 nM as determined by the protocol set out in Example 171.

In one aspect the invention provides a compound of any one of Formulae I, II, III, IV, V, VI and VII that has one or more of:
(a) an IC₅₀ value against DNA-PK of less than 500 nM as determined by quantifying the phosphorylation of a peptide substrate by human DNA-PK in the presence of DNA and ATP,
(b) a selectivity ratio of greater than 100× against one or more PI3K isoforms, and
(c) a selectivity ratio of greater than 100× against one or more PIKK kinases selected from the group consisting of mTOR, ATM and ATR.

In one embodiment the compound has an IC₅₀ value against DNA-PK of less than 400, 300, 200 or 100 nM. In one embodiment the compound has a selectivity ratio of greater than 200, 300, or 400× against one or more PI3K isoforms. In one embodiment the compound has a selectivity ratio of greater than 200, 300, or 400× against one or more PIKK kinase selected from the group consisting of mTOR, ATM and ATR.

In one aspect the invention provides a compound of any one of Formulae I, II, III or IV that has one or more of:
(a) an S50 value of less than 1 μM in combination with 3 Gy of radiation against UT-SCC-54C HNSCC cells as determined by the protocol set out in Example 175,
(b) an S50 value of less than 1 μM in combination with 3 Gy of radiation against UT-SCC-54C HNSCC cells when the ability of increasing concentrations of compound to limit tumour cell growth is measured in a cell proliferation assay,
(c) a SER₁₀ value of greater than 1.5 at 1 μM in combination with radiation against UT-SCC-54C HNSCC cells as determined by the protocol set out in Example 178, and
(d) a SER₁₀ value of greater than 1.5 at 1 μM in combination with radiation against UT-SCC-54C HNSCC cells when the ability of the compounds to sensitise tumour cells to increasing radiation doses is measured by inhibition of clonogenic survival.

In one aspect the invention provides a compound of any one of Formulae V, VI and VII that has a SER₁₀ value of greater than 1.5 under anoxic conditions as determined by the protocol set out in Example 178.

In one aspect, the invention provides a compound selected from the group consisting of: compounds 7, 8, 9, 10, 11, 12, 13, 14, 15, 16, 17, 18, 19, 20, 21, 22, 23, 24, 25, 26, 27, 28, 29, 30, 31, 32, 33, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 94, 95, 96, 97, 98, 99, 104, 105, 110, 115, 116, 121, 122, 123, 124, 125, 126, 127, 128, 129, 130, 131, 132, 133, 134, 135, 136, 141, 146, 147, 152, 153, 157, 162, 163, 167, 172, 173, 178, 179, 184, 185, 190, 195, 196, 197, 198, 199, 200, 205, 206, 211, 212, 213, 214, 215, 216, 217, 218, 219, 220, 221, 222, 223, 224, 225, 226, 230, 234, 236, 238, 240, 242, 246, 247, 248, 250, 251, 252, 253, 254, 255, 256, 258 and 260.

In one embodiment the invention the invention provides a compound selected from the group consisting of compounds 121, 125, 127, 135, 172, 225, 230, 238, 248 and 260.

The compounds of the invention may be prepared using the methods and procedures described herein or methods and procedures analogous thereto. Methods for obtaining the compounds described herein will be apparent to those of ordinary skill in the art, suitable procedures being described, for example, in the reaction schemes and references cited below. It will be appreciated that where typical or preferred process conditions (for example, reaction temperatures, times, mole ratios of reactants, solvents, pressures, etc.) are indicated, other process conditions can also be used unless otherwise stated. Optimum reaction conditions may vary with the particular reactants used.

Conventional protecting groups may be necessary to prevent certain functional groups from undergoing undesired reactions. The need for protection and deprotection and the selection of appropriate protecting groups can be readily determined by a person skilled in the art. Suitable protecting groups for various functional groups as well as suitable conditions for protecting and deprotecting particular functional groups are well known in the art (see, for example, T. W. Greene and G. M. Wuts, Protecting Groups in Organic Synthesis, Third Edition, Wiley, New York, 1999).

The starting materials useful in the methods and reactions are commercially available or can be prepared by known procedures or modifications thereof, for example those described in in standard reference texts such as Fieser and Fieser's Reagents for Organic Synthesis, Volumes 1-15 (John Wiley and Sons, 1991), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

The various starting materials, intermediates, and compounds may be isolated and purified where appropriate using conventional techniques such as precipitation, filtration, crystallization, evaporation, distillation, and chromatography. Characterization of the compounds may be performed using conventional methods such as by melting point, mass spectrum, nuclear magnetic resonance, and various other spectroscopic analyses.

For example, a compound of Formula I or a pharmacologically acceptable salt thereof,

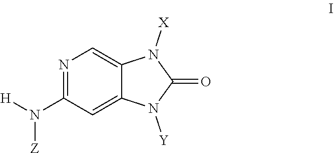

may be prepared in accordance with the following general scheme:

First, 2,4-dichloro-5-nitropyridine is reacted with an amine to prepare a compound of Formula VIII

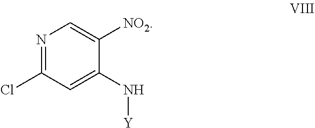

The amine may be an optionally substituted alkyl, alkenyl, cycloalkyl, heterocycloalkyl, aryl or heteroaryl amine. Additional functional groups present in the amine may be protected, according to standard protection strategies.

Next, the compound of Formula VIII is reduced using a reducing agent such as tin chloride dihydrate or zinc dust and ammonium chloride to form a compound of Formula IX wherein Y is defined as above

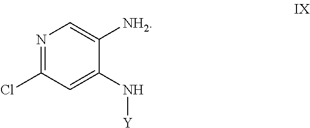

The compound of Formula IX is then reacted with carbonyldiimidazole or similar reagents to form a compound of Formula X wherein Y is defined as above

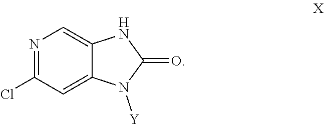

The compound of Formula X is reacted with an optionally substituted alkyl, cycloalkyl, alkenyl aryl or benzyl halide under basic conditions to form a compound of Formula XI wherein X and Y are defined as above.

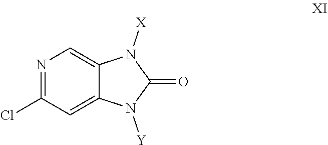

Next, the compound of Formula XI is reacted with an optionally substituted arylamine or heteroaryl amine using either acid catalysis or palladium-mediated catalysis to form a compound of Formula I.

Compounds of Formula V can be prepared by reaction of compounds of Formula I with carbamoylating agents such as phosgene, diphosgene and triphosgene to provide a stable carbamoyl chloride of Formula XII. This can be purified and isolated and reacted with various nitroaryl alcohols to form carbamates of Formula V.

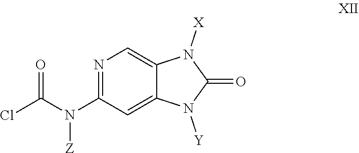

Compounds of Formula VI can be prepared from compounds of Formulae I-IV through reaction of phenolic groups with nitroheteroaryl alkyl halides under basic conditions. Further examples of compounds of Formula VI can be prepared by quaternisation of suitable tertiary amine sidechains on compounds of Formulae I-VI with nitroheteroaryl alkyl halides.

It will be appreciated that certain of the various ring substituents in the compounds of the present invention may be introduced by standard aromatic substitution reactions or generated by conventional functional group modifications either prior to or immediately following the processes mentioned above. For example, compounds of Formula I may be converted into further compounds of Formula I by standard aromatic substitution reactions or by conventional functional group modifications. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel-Crafts conditions; the introduction of an alkyl group using an alkyl halide and Lewis acid (such as aluminium trichloride) under Friedel-Crafts conditions; and the introduction of a halogen group. Particular examples of modifications include the reduction of a nitro group to an amino group by, for example, catalytic hydrogenation with a nickel catalyst or treatment with iron in the presence of hydrochloric acid with heating; oxidation of alkylthio to alkylsulfinyl or alkylsulfonyl.

Referring to Scheme 1 below, the treatment of 4-chloro-5-nitropyridin-2-ol (1) with phosphorus oxychloride and tetramethylammonium chloride gave the dichloride 2 (Scheme 1). Displacement of the chloride 2 with cyclopentylamine gave nitroamine 3 and reduction of 3 with tin chloride dihydrate gave the diamine 4. Reaction of 4 with carbonyldiimidazole provided the pyridoimidazolone 5 which was alkylated using sodium hydride and methyl iodide to give chloride 6. Displacement of the chloride of 6 with aniline using Buchwald conditions provided the compound 7.

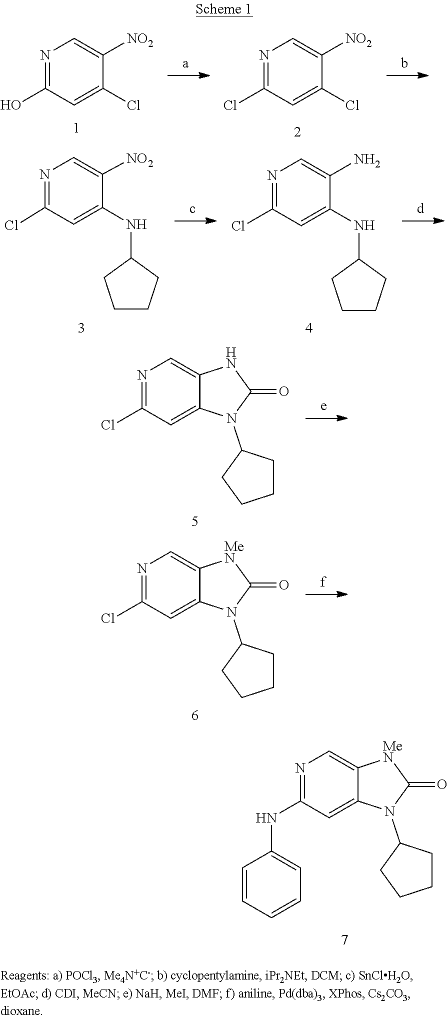

Similarly, reaction of chloride 6 with a variety of anilines and heteroarylamines using Buchwald conditions gave compounds 8-81 (Scheme 2).

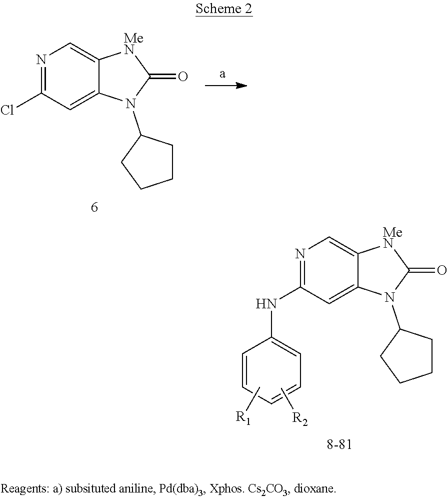

TABLE 1
Compounds 8-81

| SN | No. | Reagent | R1 | R2 |
|---|---|---|---|---|
| 39229 | 8 | p-toluidine | 4-Me | H |
| 39231 | 9 | m-toluidine | 3-Me | H |
| 39232 | 10 | o-toluidine | 2-Me | H |
| 39236 | 11 | p-anisidine | 4-OMe | H |
| 39239 | 12 | m-anisidine | 3-OMe | H |
| 39240 | 13 | o-anisidine | 2-OMe | H |
| 39241 | 14 | 4-chloroaniline | 4-Cl | H |
| 39242 | 15 | 3-chloroaniline | 3-Cl | H |
| 39245 | 16 | 2-chloroaniline | 2-Cl | H |
| 39246 | 17 | 4-nitroaniline | 4-NO₂ | H |
| 39247 | 18 | 3-nitroaniline | 3-NO₂ | H |
| 39263 | 19 | 2-nitroaniline | 2-NO₂ | H |
| 39273 | 20 | N-(4-aminophenyl)acetamide | 4-NHAc | H |
| 39280 | 21 | N-(3-aminophenyl)acetamide | 3-NHAc | H |
| 39301 | 22 | N-(2-aminophenyl)acetamide | 2-NHAc | H |
| 39275 | 23 | 4-aminobenzonitrile | 4-CN | H |
| 39291 | 24 | 3-aminobenzonitrile | 3-CN | H |
| 39297 | 25 | 2-aminobenzonitrile | 2-CN | H |
| 39278 | 26 | 4-(trifluoromethyl)aniline | 4-CF₃ | H |
| 39290 | 27 | 3-(trifluoromethyl)aniline | 3-CF₃ | H |
| 39283 | 28 | 2-(trifluoromethyl)aniline | 2-CF₃ | H |
| 39274 | 29 | 1-(4-aminophenyl)ethan-1-one | 4-Ac | H |
| 39279 | 30 | 1-(3-aminophenyl)ethan-1-one | 3-Ac | H |
| 39304 | 31 | 4-(methylsulfonyl)aniline | 4-SO₂Me | H |
| 39308 | 32 | 3-(methylsulfonyl)aniline | 3-SO₂Me | H |
| 39330 | 33 | 2-(methylsulfonyl)aniline | 2-SO₂Me | H |
| 39302 | 34 | 4-(benzyloxy)aniline | 4-OBn | H |
| 39303 | 35 | 3-(benzyloxy)aniline | 3-OBn | H |
| 39328 | 36 | 2-(benzyloxy)aniline | 2-OBn | H |
| 39309 | 37 | 2-(4-aminophenyl)-2- | 4-(2- | H |
| | | methylpropanenitrile | methyl | |
| | | | propane- | |
| | | | nitrile) | |
| 39385 | 38 | 2,4-dimethylaniline | 2-Me | 4-Me |
| 39390 | 39 | 2,3-dimethylaniline | 2-Me | 3-Me |
| 39393 | 40 | 3,4-dimethylaniline | 3-Me | 4-Me |
| 39378 | 41 | 3-chloro-2-methylaniline | 2-Me | 3-Cl |
| 39376 | 42 | 4-chloro-2-methylaniline | 2-Me | 4-Cl |
| 39379 | 43 | 5-chloro-2-methylaniline | 2-Me | 5-Cl |
| 39396 | 44 | 2-methyl-5-(methyl- | 2-Me | 5-SO₂Me |
| | | sulfonyl)aniline | | |
| 39397 | 45 | 2-methyl-4-(methyl- | 2-Me | 4-SO₂Me |
| | | sulfonyl)aniline | | |
| 39398 | 46 | 4-amino-3-methylbenzonitrile | 2-Me | 4-CN |
| 39401 | 47 | 4-amino-2-methylbenzonitrile | 3-Me | 4-CN |
| 39369 | 48 | 4-methoxy-2-methylaniline | 2-Me | 4-OMe |
| 39382 | 49 | 5-methoxy-2-methylaniline | 2-Me | 5-OMe |
| 39474 | 50 | 4-methoxy-N,2-dimethylaniline | 2-Me, | 4-OMe |
| | | | NMe | |
| 39370 | 51 | 4-benzyloxy-2-methylaniline | 2-Me | 4-OBn |
| 39642 | 52 | 4-fluoro-2-methylaniline | 2-Me | 4-F |
| 39748 | 53 | 2-methyl-4-(trifluoro- | 2-Me | 4OCF₃ |
| | | methoxy)aniline | | |
| 39761 | 54 | 4-(difluoromethoxy)-2- | 2-Me | 4-CHF₂ |
| | | methylaniline | | |
| 39764 | 55 | 4-ethoxy-2-methylaniline | 2-Me | 4-OEt |
| 39673 | 56 | 4-(2-(dimethylamino)ethoxy)- | 2-Me | 4-OCH₂ |
| | | 2-methylphenyl)amino | | CH₂—NMe₂ |
| 39663 | 57 | 2-methyl-4-morpholinoaniline | 2-Me | 4-morpholine |
| 39637 | 58 | 2-methyl-4-(4-methylpiperazin- | 2-Me | 4-(4′-methyl |
| | | 1-yl)aniline | | piperazine) |
| 39758 | 59 | 4-methoxy-2-(trifluoro- | 2-CF₃ | 4-OMe |
| | | methyl)aniline | | |
| 39762 | 60 | 2-amino-5-methoxybenzonitrile | 2-CN | 4-OMe |
| 39759 | 61 | 2-chloro-4-methoxyaniline | 2-Cl | 4-OMe |
| 39717 | 62 | 2,4-dimethoxyaniline | 2-OMe | 4-OMe |

| | | | |
|---|---|---|---|
| 39313 | 63 | quinolin-6-amine | 3-CH═CHCH═N-4 |
| 39316 | 64 | quinolin-3-amine | 3-aza, 4-CH═ |
| | | | CHCH═CH-5 |
| 39322 | 65 | quinolin-5-amine | 2-CH═CHCH═N-3 |
| 39323 | 66 | isoquinolin-5-amine | 2-CH═CHN═CH-3 |
| 39325 | 67 | 2-methylquinolin-4-amine | 4-aza, 2-CH═CHCH═ |
| | | | CH-3, 5-Me |
| 39326 | 68 | quinoxalin-6-amine | 2-N═CHCH═N-3 |
| 39402 | 69 | benzo[d][1,3]dioxol-5-amine | 3-OCH₂O-4 |
| 39441 | 70 | benzo[d]thiazol-6-amine | 3-SCH═N-4 |
| 39333 | 71 | pyridin-4-amine | 4-aza |
| 39334 | 72 | pyridin-3-amine | 3-aza |
| 39341 | 73 | pyridin-2-amine | 2-aza |
| 39344 | 74 | 3-methylpyridin-4-amine | 4-aza, 2-Me |
| 39361 | 75 | 2-methylpyridin-4-amine | 4-aza, 3-Me |
| 39346 | 76 | 2-methylpyridin-3-amine | 3-aza, 2-Me |
| 39362 | 77 | 5-methylpyridin-3-amine | 3-aza, 5-Me |
| 39342 | 78 | 4-methylpyridin-3-amine | 3-aza, 6-Me |
| 39360 | 79 | 3-methylpyridin-2-amine | 2-aza, 6-Me |
| 39405 | 80 | 6-methoxy-4-methyl- | 3-aza, 2-Me, 4-OMe |
| | | pyridin-3-amine | |
| 39395 | 81 | 6-methoxypyrimidin-4-amine | 2-aza, 4-aza, 5-OMe |

Reduction of the nitro-substituted compounds 14-16 with Pd/C under hydrogen gave the corresponding anilines 82-84, respectively (Scheme 3).

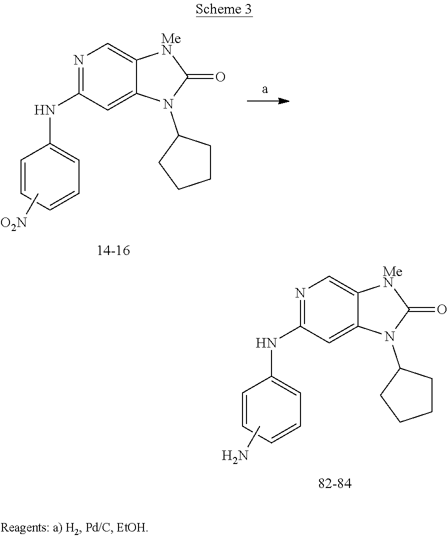

TABLE 2
Compounds 82-84

| SN | No. | R |
|---|---|---|
| 39258 | 82 | 4-NH₂ |
| 39259 | 83 | 3-NH₂ |
| 39272 | 84 | 2-NH₂ |

Reduction of the benzyl ethers 34-36 with Pd/C under hydrogen gave the corresponding phenols 85-87, respectively (Scheme 4). Similarly, reduction of benzyl ether 51 gave phenol 88.

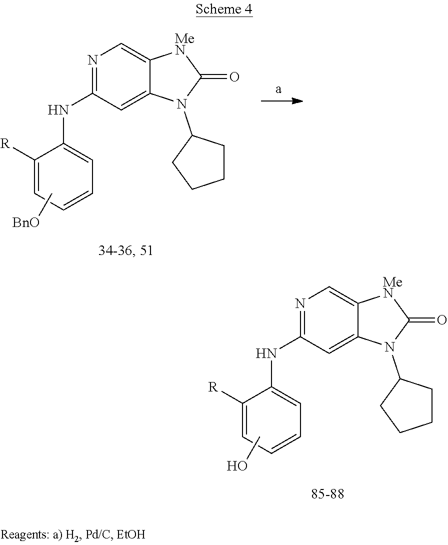

TABLE 3
Compounds 85-88

| SN | No. | R |
|---|---|---|
| 39305 | 85 | 4-OH, R═H |
| 39306 | 86 | 3-OH, R═H |
| 39329 | 87 | 2-OH, R═H |
| 39375 | 88 | 4-OH, R═Me |

Alkylation of imidazolone 5 with NaH and various alkyl halides gave the chlorides 89-93 (Scheme 5). Displacement of chlorides 89-93 under Buchwald conditions gave the corresponding imidazopyridinones 94-98. Reduction of benzyl ether 98 gave alcohol 99.

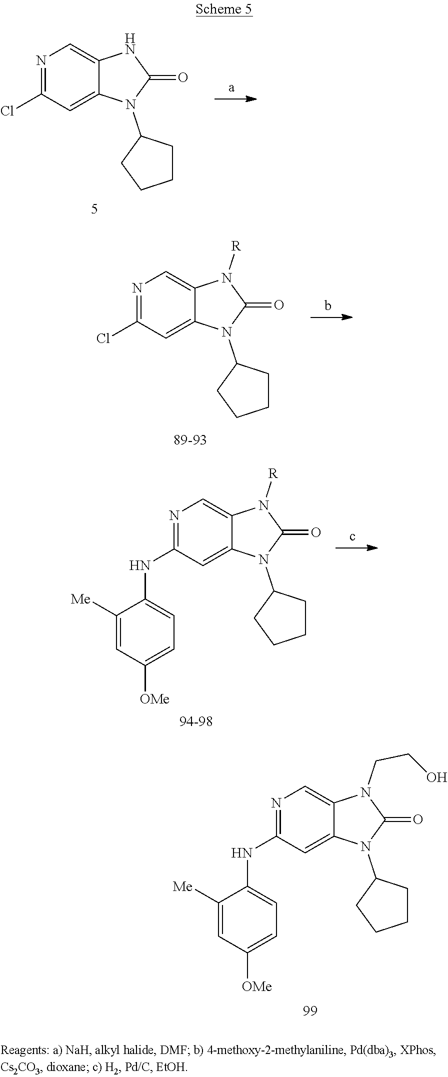

TABLE 4
Compounds 94-99

| SN | No. | Reagent | R |
|---|---|---|---|
| 39777 | 94 | Benzyl bromide | Bn |
| 39778 | 95 | Cyclopentyl iodide | cyclopentyl |
| 39790 | 96 | Isopropyl bromide | isopropyl |
| 39789 | 97 | MeOCH₂CH₂Br | CH₂CH₂OMe |
| 39793 | 98 | BnOCH₂CH₂Br | CH₂CH₂OBn |
| 39794 | 99 | Pd/C | CH₂CH₂OH |

Displacement of the chloride 2 with 2-methoxyethylamine gave nitroamine 100 and reduction of 100 with tin chloride dihydrate gave the diamine 101 (Scheme 6). Reaction of 101 with carbonyldiimidazole provided the pyridoimidazolone 102 which was alkylated using sodium hydride and methyl iodide to give chloride 103. Displacement of the chloride of 103 with 4-methoxy-2-methylaniline or 4-chloro-2-methylaniline using Buchwald conditions provided the compounds 104 (SN39478) and 105 (SN39551), respectively.

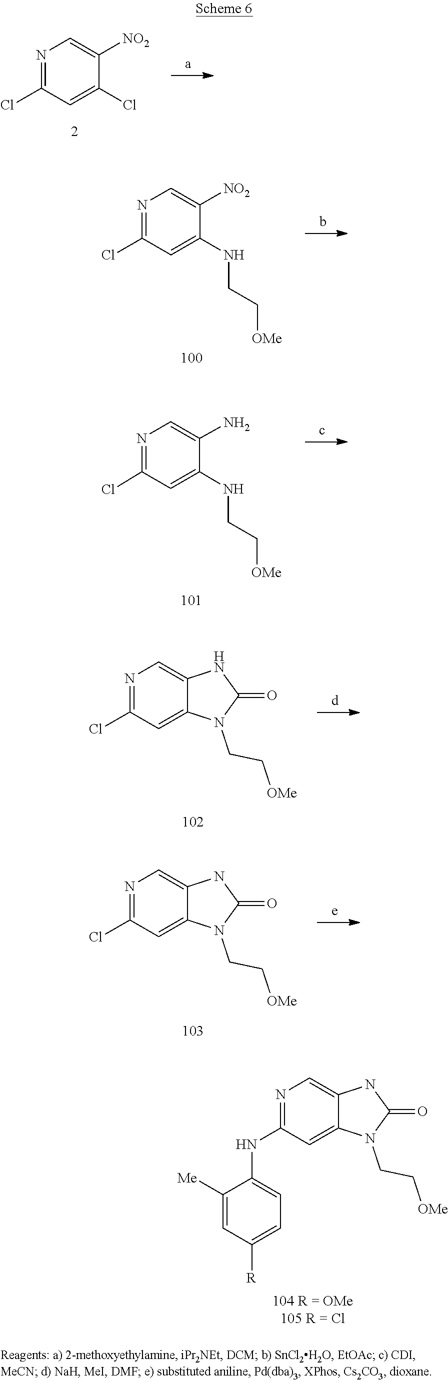

Displacement of the chloride 2 with oxetan-3-amine gave nitroamine 106 and reduction of 106 with tin chloride dihydrate gave the diamine 107 (Scheme 7). Reaction of 107 with carbonyldiimidazole provided the pyridoimidazolone 108 which was alkylated using sodium hydride and methyl iodide to give chloride 109. Displacement of the chloride of 109 with 4-methoxy-2-methylaniline using Buchwald conditions provided the compound 110.

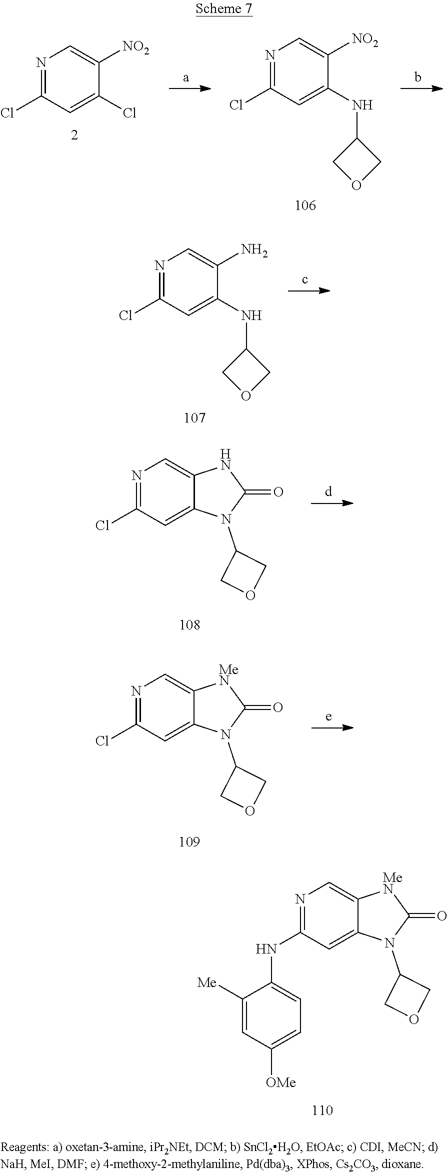 [SMILES: O=[N+]([O-])c1cnc(Cl)cc1Cl ; O=[N+]([O-])c1cnc(Cl)cc1NC1COC1 ; Nc1cnc(Cl)cc1NC1COC1 ; O=c1[nH]c2cnc(Cl)cc2n1C1COC1 ; Cn1c(=O)n(C2COC2)c2cc(Cl)ncc21 ; COc1ccc(Nc2cc3c(cn2)n(C)c(=O)n3C2COC2)c(C)c1]

Displacement of the chloride 2 with tetrahydrofuran-3-amine gave nitroamine 111 and reduction of 111 with tin chloride dihydrate gave the diamine 112 (Scheme 8). Reaction of 112 with carbonyldiimidazole provided the pyridoimidazolone 113 which was alkylated using sodium hydride and methyl iodide to give chloride 114. Displacement of the chloride of 114 with 4-methoxy-2-methylaniline or 4-chloro-2-methylaniline using Buchwald conditions provided the compounds 115 (SN39878) and 116 (SN39881), respectively.

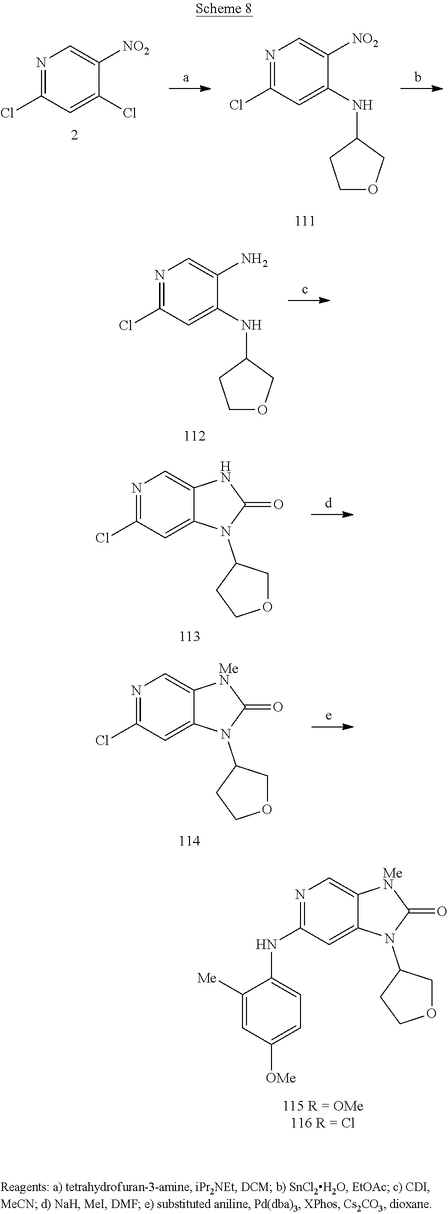 [SMILES: O=[N+]([O-])c1cnc(Cl)cc1Cl ; O=[N+]([O-])c1cnc(Cl)cc1NC1CCOC1 ; Nc1cnc(Cl)cc1NC1CCOC1 ; O=c1[nH]c2cnc(Cl)cc2n1C1CCOC1 ; Cn1c(=O)n(C2CCOC2)c2cc(Cl)ncc21 ; COc1ccc(Nc2cc3c(cn2)n(C)c(=O)n3C2CCOC2)c(C)c1]

Displacement of the chloride 2 with tetrahydro-2H-pyran-4-amine gave nitroamine 117 and reduction of 117 with tin chloride dihydrate gave the diamine 118 (Scheme 9). Reaction of 118 with carbonyldiimidazole provided the pyridoimidazolone 119 which was alkylated using sodium hydride and methyl iodide to give chloride 120. Displacement of the chloride of 120 with various amines using Buchwald conditions provided the compounds 121-132.

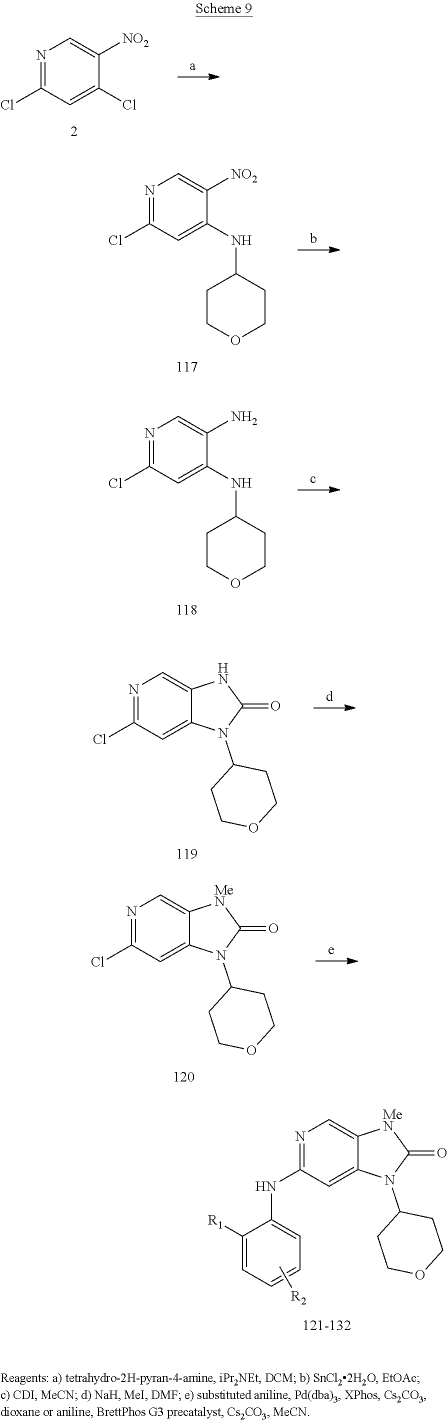

TABLE 5
Compounds 121-132

| SN | No. | Reagent | R1 | R2 |
|---|---|---|---|---|
| 39536 | 121 | 4-methoxy-2-methylaniline | Me | 4-OMe |
| 39537 | 122 | 4-chloro-2-methylaniline | Me | 4-Cl |
| 39538 | 123 | 2-methyl-5-(methylsulfonyl)aniline | Me | 5-SO₂Me |
| 39871 | 124 | 4-benzyloxy-2-methylaniline | Me | 4-OBn |
| 40019 | 125 | 7-methyl-[1,2,4]triazolo[1,5-a] | Me | 5-aza, 2-Me, 4═NCH═N-5 |
| | | pyridin-6-amine | | |
| 40037 | 126 | 6-methoxy-4-methylpyridin-3-ami [symbol] | Me | 5-aza, 4-OMe |
| 40046 | 127 | 2,5-dimethylbenzo[d]thiazol-6- | Me | 4-N═C(Me)S-5 |
| | | amine | | |
| 40050 | 128 | 2-methylbenzo[d]oxazol-6-amine | H | 4-N═C(Me)O-5 |
| 40070 | 129 | ethyl 6-amino-7-methylimidazo[1,2 [symbol] | Me | 5-aza, 4═NCH═C(CO₂Et)-5 |
| | | a]pyridine-3-carboxylate | | |
| 40092 | 130 | 6-methylbenzo[d][1,3]dioxol-5-amine | Me | 4-OCH₂O-5 |
| 40151 | 131 | 2,6-dimethylbenzo[d]oxazol-5-amine | Me | 4-OC(Me)═N-5 |
| 40152 | 132 | 2,5-dimethylbenzo[d]oxazol-6-amine | Me | 4═NC(Me)O-5 |

[symbol]

indicates data missing or illegible when filed

Reaction of imidazopyridinones 121 and 122 with benzyl chloroformate and iPr2NEt gave the corresponding carbamates 133 (SN39689) and 134 (SN39690)(Scheme 10).

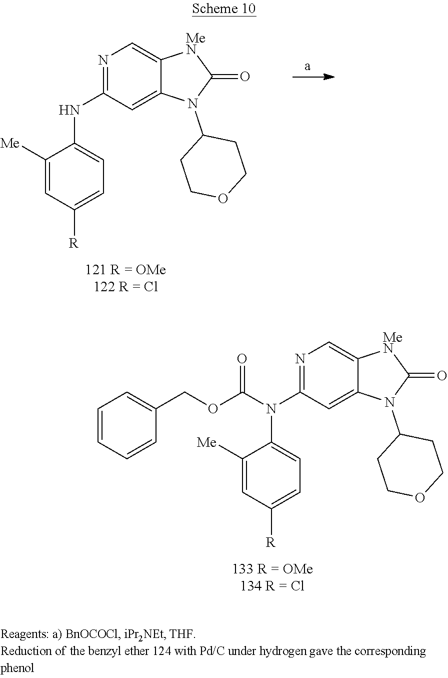

135 (SN39872) (Scheme 11).

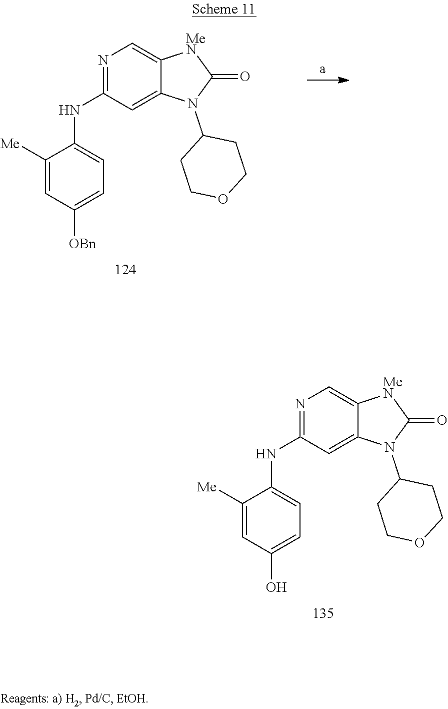 [SMILES: Cc1cc(OCc2ccccc2)ccc1Nc1cc2c(cn1)n(C)c(=O)n2C1CCOCC1 ; Cc1cc(O)ccc1Nc1cc2c(cn1)n(C)c(=O)n2C1CCOCC1]

Hydrolysis of ester 129 under basic conditions gave the acid 136 (SN40071)(Scheme 12).

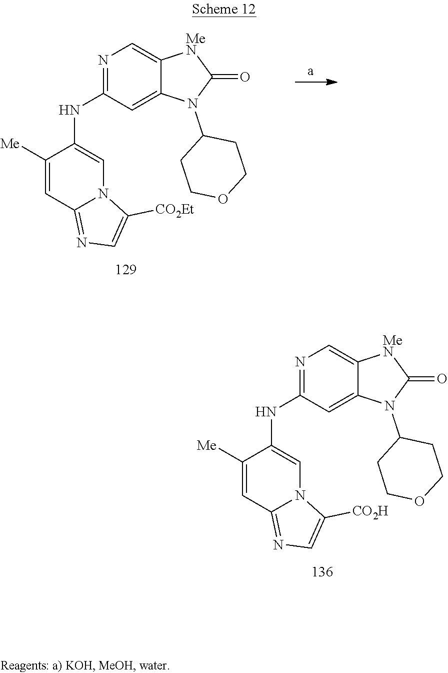 [SMILES: CCOC(=O)c1cnc2cc(C)c(Nc3cc4c(cn3)n(C)c(=O)n4C3CCOCC3)cn12 ; Cc1cc2ncc(C(=O)O)n2cc1Nc1cc2c(cn1)n(C)c(=O)n2C1CCOCC1]

Displacement of the chloride 2 with (tetrahydro-2H-pyran-4-yl)methanamine gave nitroamine 137 and reduction of 137 with zinc powder and ammonium chloride gave the diamine 138 (Scheme 13). Reaction of 138 with carbonyldiimidazole provided the pyridoimidazolone 139 which was alkylated using sodium hydride and methyl iodide to give chloride 140. Displacement of the chloride of 140 with 4-methoxy-2-methylaniline, using Buchwald conditions, provided the compound 141 (SN39667).

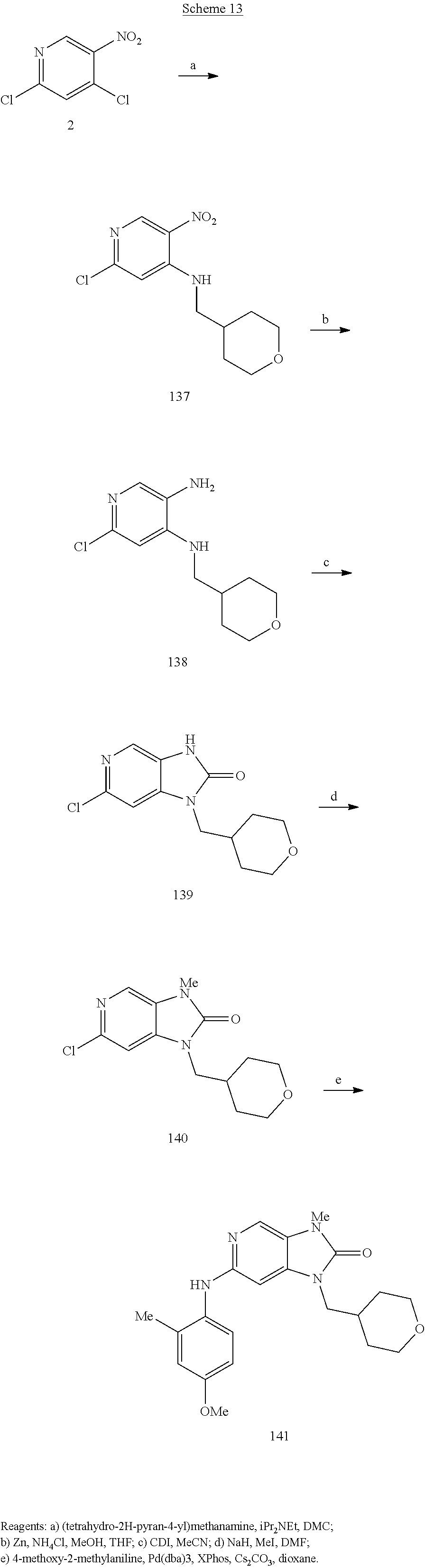 [SMILES: O=[N+]([O-])c1cnc(Cl)cc1Cl ; O=[N+]([O-])c1cnc(Cl)cc1NCC1CCOCC1 ; Nc1cnc(Cl)cc1NCC1CCOCC1 ; O=c1[nH]c2cnc(Cl)cc2n1CC1CCOCC1 ; Cn1c(=O)n(CC2CCOCC2)c2cc(Cl)ncc21 ; COc1ccc(Nc2cc3c(cn2)n(C)c(=O)n3CC2CCOCC2)c(C)c1]

Displacement of the chloride 2 with 2-(tetrahydro-2H-pyran-4-yl)ethan-1-amine gave nitroamine 142 and reduction of 142 with zinc powder and ammonium chloride gave the diamine 143 (Scheme 14). Reaction of 143 with carbonyldiimidazole provided the pyridoimidazolone 144 which was alkylated using sodium hydride and methyl iodide to give chloride 145. Displacement of the chloride of 145 with 4-methoxy-2-methylaniline or 4-chloro-2-methylaniline, using Buchwald conditions, provided the compounds 146 (SN39550) and 147 (SN39552), respectively.

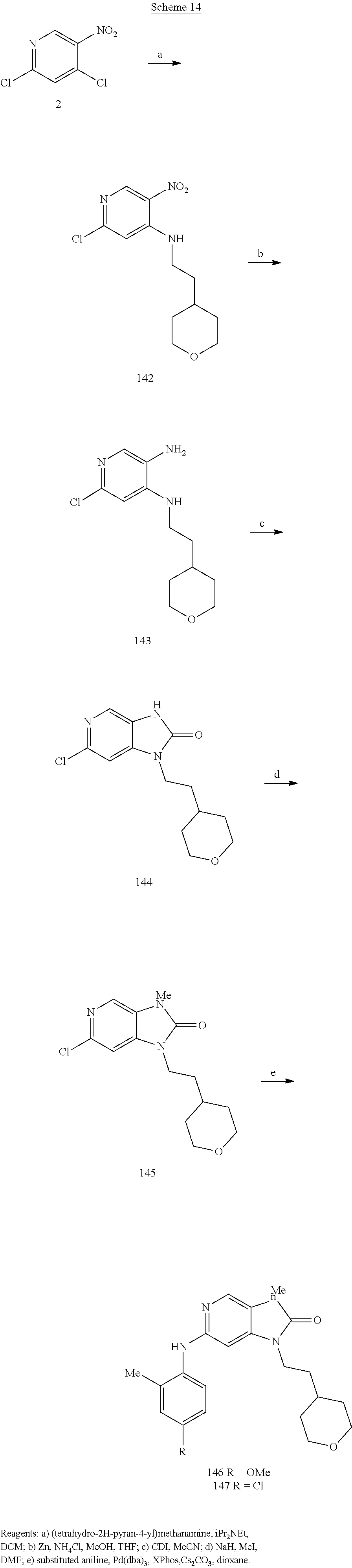

Displacement of the chloride 2 with tert-butyl 4-aminopiperidine-1-carboxylate gave nitroamine 148 and reduction of 148 with zinc powder and ammonium chloride gave the diamine 149 (Scheme 15). Reaction of 149 with carbonyldiimidazole provided the pyridoimidazolone 150 which was alkylated using sodium hydride and methyl iodide to give chloride 151. Displacement of the chloride of 151 with 4-methoxy-2-methylaniline, using Buchwald conditions, provided carbamate 152 (SN39598). Hydrolysis of carbamate 152 under acidic conditions gave the compound 153 (SN39600) as the hydrochloride salt.

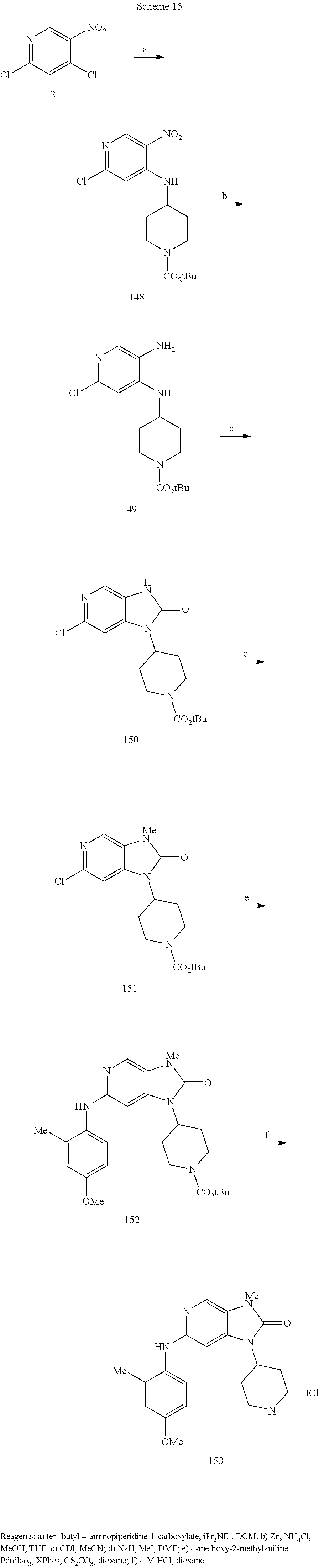

Reaction of imidazopyridinone 152 with benzoyl chloroformate gave the carbamate 154 (Scheme 16). Acid hydrolysis of 154 gave the amine 155. Reductive amination of 155 with formaldehyde and sodium triacetoxyborohydride gave amine 156 which was converted to compound 157 (SN39686) under reductive conditions.

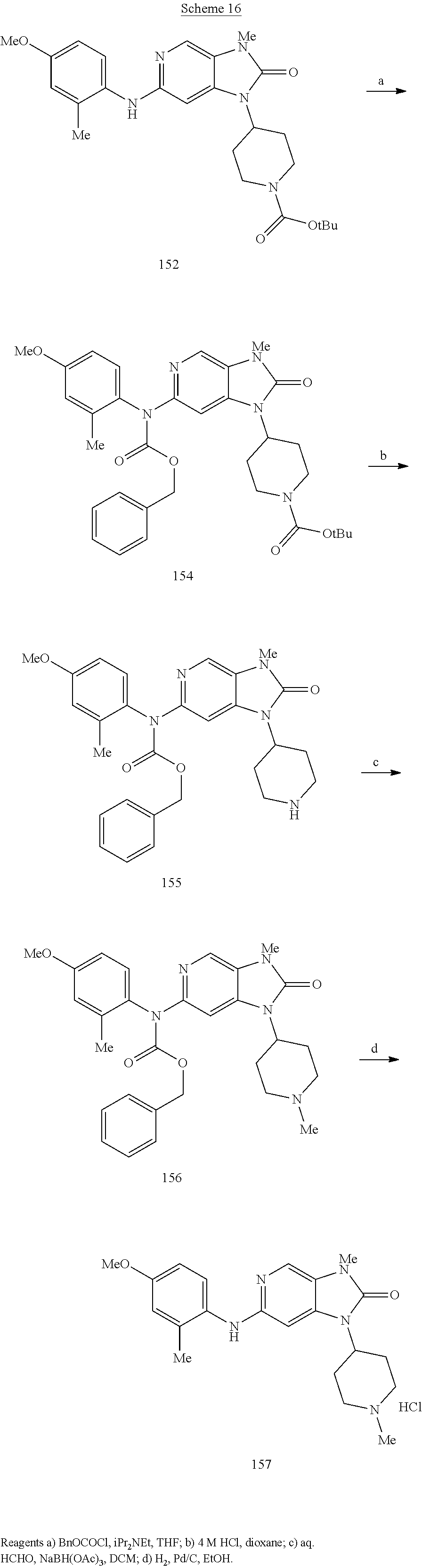 [SMILES: COc1ccc(Nc2cc3c(cn2)n(C)c(=O)n3C2CCN(C(=O)OC(C)(C)C)CC2)c(C)c1 ; COc1ccc(N(C(=O)OCc2ccccc2)c2cc3c(cn2)n(C)c(=O)n3C2CCN(C(=O)OC(C)(C)C)CC2)c(C)c1 ; COc1ccc(N(C(=O)OCc2ccccc2)c2cc3c(cn2)n(C)c(=O)n3C2CCNCC2)c(C)c1 ; COc1ccc(N(C(=O)OCc2ccccc2)c2cc3c(cn2)n(C)c(=O)n3C2CCN(C)CC2)c(C)c1 ; COc1ccc(Nc2cc3c(cn2)n(C)c(=O)n3C2CCN(C)CC2)c(C)c1 ; Cl]

Displacement of the chloride 2 with tert-butyl 4-(aminomethyl)piperidine-1-carboxylate gave nitroamine 158 and reduction of 158 with zinc powder and ammonium chloride gave the diamine 159 (Scheme 17). Reaction of 159 with carbonyldiimidazole provided the pyridoimidazolone 160 which was alkylated using sodium hydride and methyl iodide to give chloride 161. Displacement of the chloride of 161 with 4-methoxy-2-methylaniline, using Buchwald conditions, provided carbamate 162 (SN39627). Hydrolysis of carbamate 162 gave amine 163 (SN39628) as the hydrochloride salt.

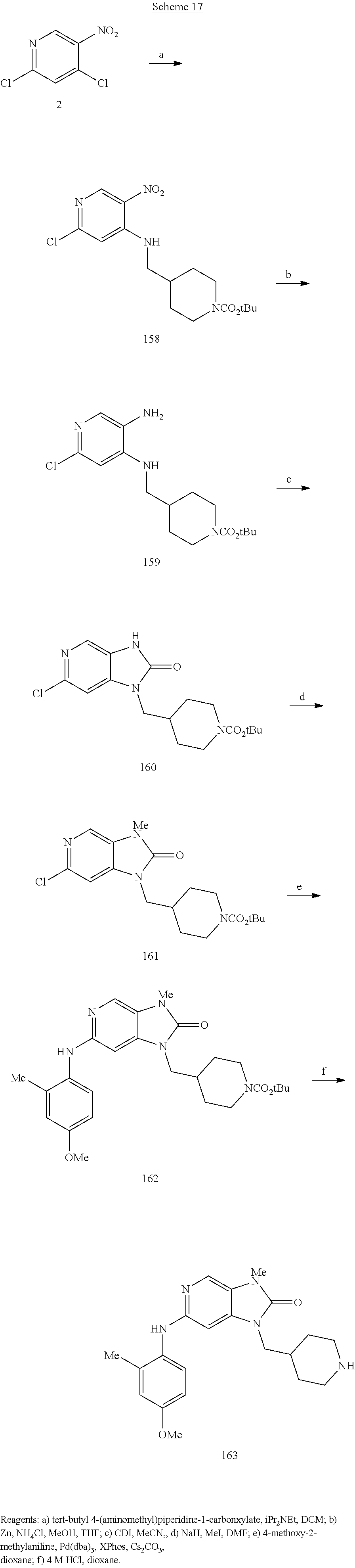

Reaction of imidazopyridinone 162 with benzoyl chloroformate gave the carbamate 164 (Scheme 18). Acid hydrolysis of 164 gave the amine 165. Reductive amination of 165 with formaldehyde and sodium triacetoxyborohydride gave amine 166 which was converted to compound 167 (SN39687) under reductive conditions.

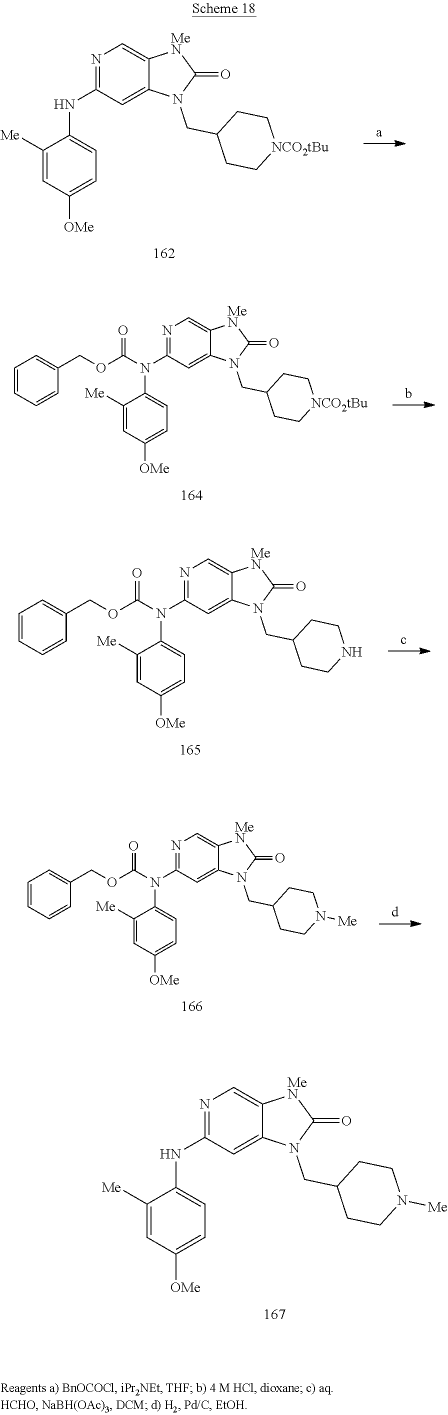

Displacement of the chloride 2 with 4-methoxycyclohexan-1-amine gave nitroamine 168 and reduction of 168 with tin chloride dihydrate gave the diamine 169 (Scheme 19). Reaction of 169 with carbonyldiimidazole provided the pyridoimidazolone 170 which was alkylated using sodium hydride and methyl iodide to give chloride 171. Displacement of the chloride of 171 with 4-methoxy-2-methylaniline or 4-chloro-2-methylaniline, using Buchwald conditions, provided the compounds 172 (SN39540) and 173 (SN39539), respectively.

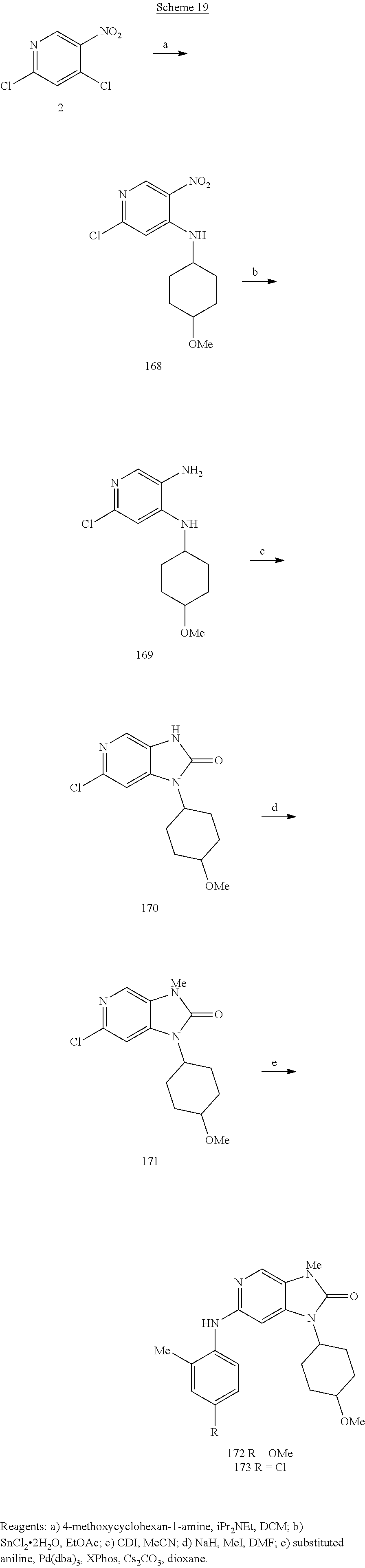

Displacement of the chloride 2 with 4-(benzyloxy)cyclohexan-1-amine gave nitroamine 174 and reduction of 174 with tin chloride dihydrate gave the diamine 175 (Scheme 20). Reaction of 175 with carbonyldiimidazole provided the pyridoimidazolone 176 which was alkylated using sodium hydride and methyl iodide to give chloride 177. Displacement of the chloride of 177 with 4-methoxy-2-methylaniline, using Buchwald conditions, provided the compound 178 (SN39581). Hydrogenolysis of the benzyl ether 178 gave the alcohol 179 (SN39584).

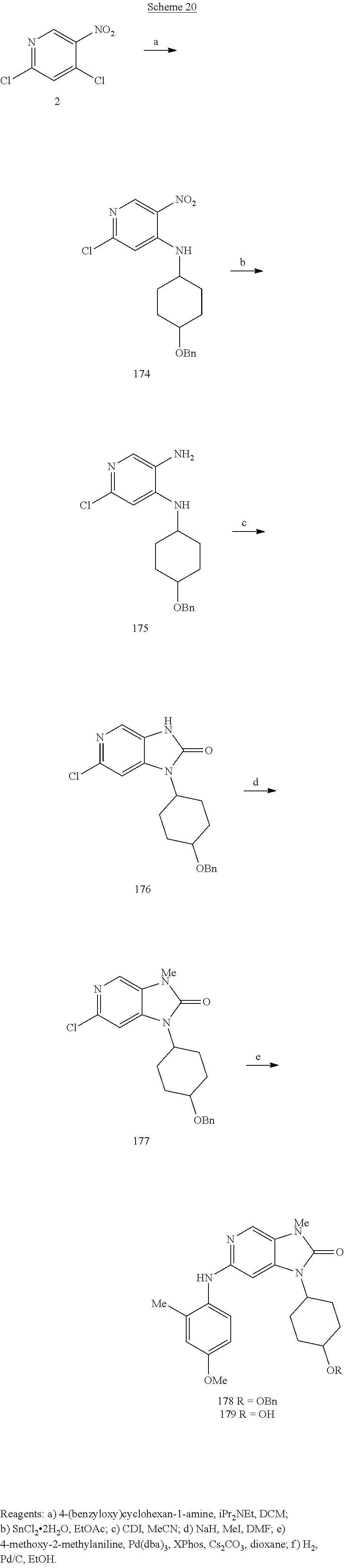

Displacement of the chloride 2 with tert-butyl (4-aminocyclohexyl)carbamate gave nitroamine 180 and reduction of 180 with zinc powder and ammonium formate gave the diamine 181 (Scheme 21). Reaction of 181 with carbonyldiimidazole provided the pyridoimidazolone 182 which was alkylated using sodium hydride and methyl iodide to give chloride 183. Displacement of the chloride of 183 with 4-methoxy-2-methylaniline, using Buchwald conditions, provided the compound 184. Acid hydrolysis of the carbamate 184 (SN40297) gave the amine 185 (SN39695).

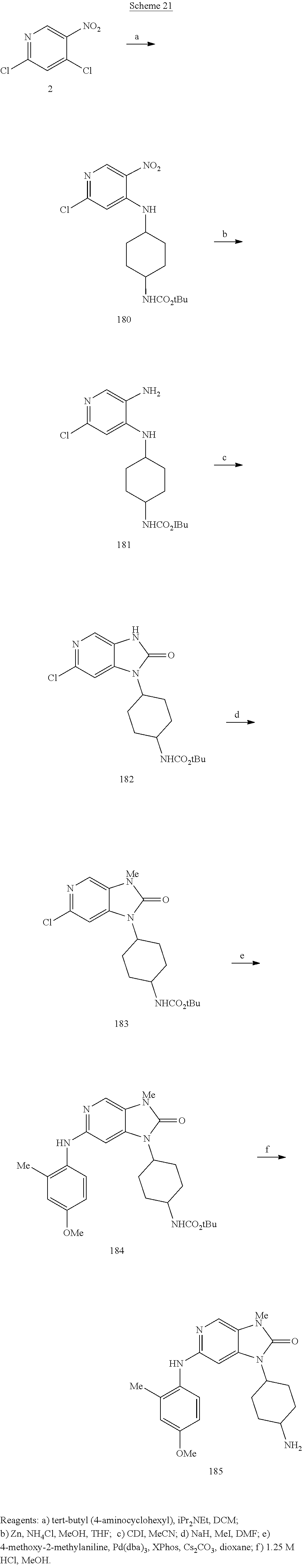

Displacement of the chloride 2 with aniline gave nitroamine 186 and reduction of 186 with tin chloride dihydrate gave the diamine 187 (Scheme 22). Reaction of 187 with carbonyldiimidazole provided the pyridoimidazolone 188 which was alkylated using sodium hydride and methyl iodide to give chloride 189. Displacement of the chloride of 189 with 4-methoxy-2-methylaniline, using Buchwald conditions, provided the compound 190 (SN39623).

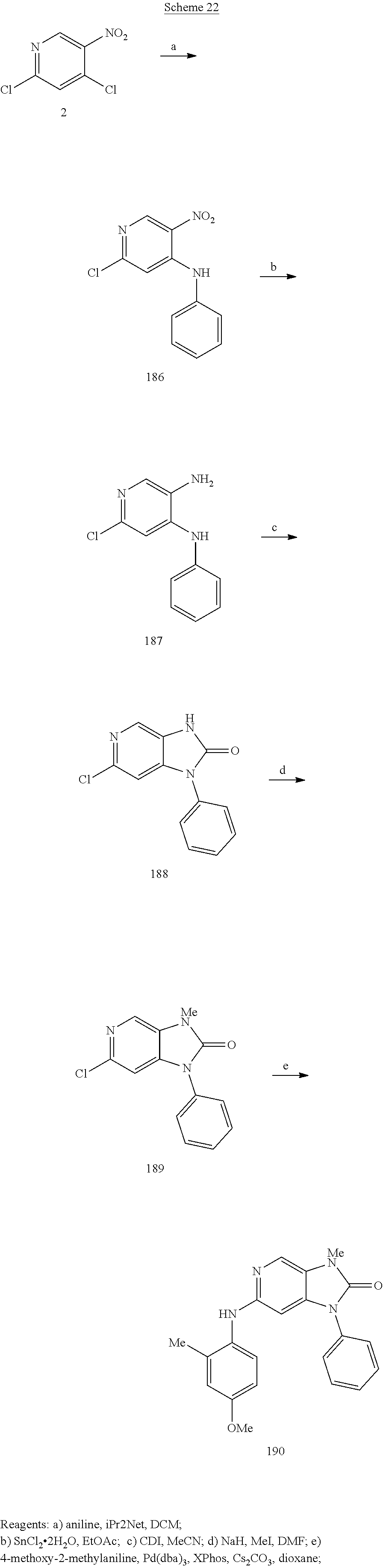 [SMILES: O=[N+]([O-])c1cnc(Cl)cc1Cl ; O=[N+]([O-])c1cnc(Cl)cc1Nc1ccccc1 ; Nc1cnc(Cl)cc1Nc1ccccc1 ; O=c1[nH]c2cnc(Cl)cc2n1-c1ccccc1 ; Cn1c(=O)n(-c2ccccc2)c2cc(Cl)ncc21 ; COc1ccc(Nc2cc3c(cn2)n(C)c(=O)n3-c2ccccc2)c(C)c1]

Displacement of the chloride 2 with anisidine gave nitroamine 191 and reduction of 191 with tin chloride dihydrate gave the diamine 192 (Scheme 23). Reaction of 192 with carbonyldiimidazole provided the pyridoimidazolone 193 which was alkylated using sodium hydride and methyl iodide to give chloride 194. Displacement of the chloride of 194 with various anilines, using Buchwald conditions, provided the compounds 195-199.

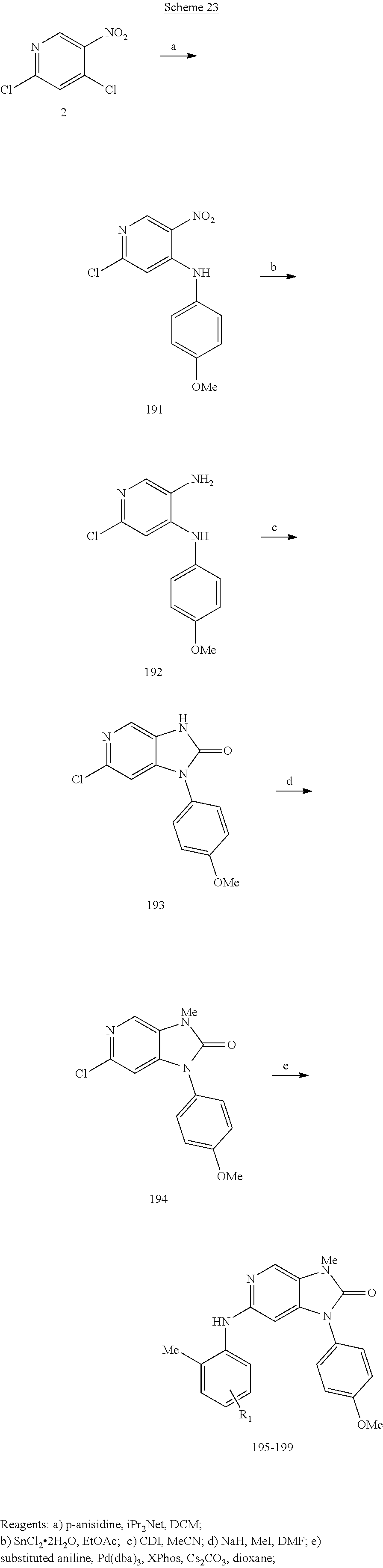

TABLE 6
Compounds 195-199

| SN | No. | Reagent | R1 |
|---|---|---|---|
| 39519 | 195 | 4-methoxy-2-methylaniline | 4-OMe |
| 39529 | 196 | 4-chloro-2-methylaniline | 4-Cl |
| 39522 | 197 | 2-methyl-5-(methylsulfonyl)aniline | 5-SO₂Me |
| 39520 | 198 | 2-methyl-4-(methylsulfonyl)aniline | 4-SO₂Me |
| 39526 | 199 | 4-benzyloxy-2-methylaniline | 4-OBn |

Reduction of the benzyl ether 199 with Pd/C under hydrogen gave the corresponding phenol 200 (SN39530)(Scheme 24).

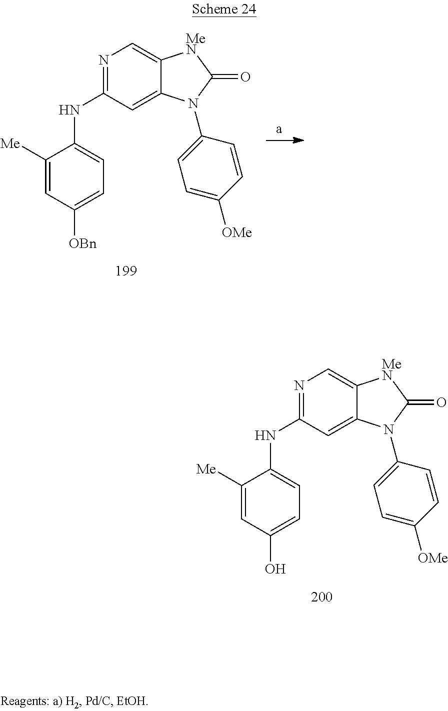 [SMILES: COc1ccc(-n2c(=O)n(C)c3cnc(Nc4ccc(OCc5ccccc5)cc4C)cc32)cc1 ; COc1ccc(-n2c(=O)n(C)c3cnc(Nc4ccc(O)cc4C)cc32)cc1]

Displacement of the chloride 2 with 4-(benzyloxy)aniline gave nitroamine 201 and reduction of 201 with tin chloride dihydrate gave the diamine 202 (Scheme 25). Reaction of 202 with carbonyldiimidazole provided the pyridoimidazolone 203 which was alkylated using sodium hydride and methyl iodide to give chloride 204. Displacement of the chloride of 204 with 4-methoxy-2-methylaniline, using Buchwald conditions, provided the compound 205 (SN39525). Reduction of the benzyl ether 205 with Pd/C under hydrogen gave the corresponding phenol 206 (SN39528).

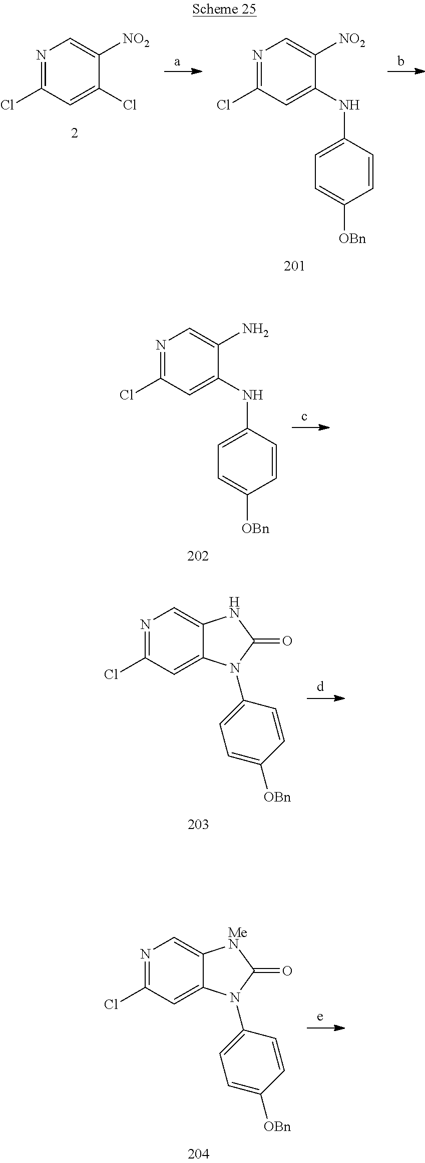 [SMILES: O=[N+]([O-])c1cnc(Cl)cc1Cl ; O=[N+]([O-])c1cnc(Cl)cc1Nc1ccc(OCc2ccccc2)cc1 ; Nc1cnc(Cl)cc1Nc1ccc(OCc2ccccc2)cc1 ; O=c1[nH]c2cnc(Cl)cc2n1-c1ccc(OCc2ccccc2)cc1 ; Cn1c(=O)n(-c2ccc(OCc3ccccc3)cc2)c2cc(Cl)ncc21]

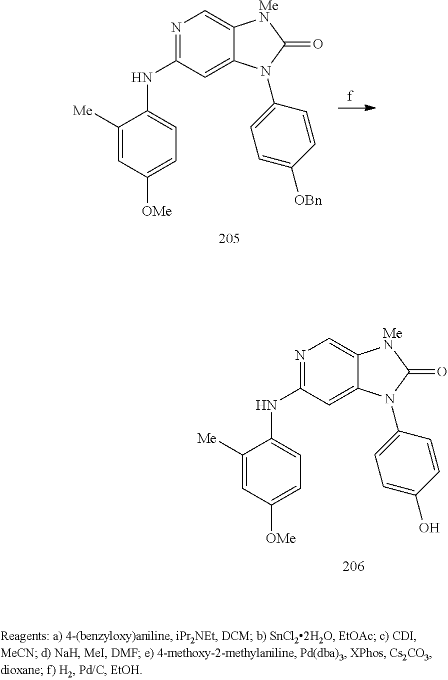 [SMILES: COc1ccc(Nc2cc3c(cn2)n(C)c(=O)n3-c2ccc(OCc3ccccc3)cc2)c(C)c1 ; COc1ccc(Nc2cc3c(cn2)n(C)c(=O)n3-c2ccc(O)cc2)c(C)c1]

Displacement of the chloride 2 with 2-(4-aminophenyl)-2-methylpropanenitrile gave nitroamine 207 and reduction of 207 with tin chloride dihydrate gave the diamine 208 (Scheme 26).

Reaction of 208 with carbonyldiimidazole provided the pyridoimidazolone 209 which was alkylated using sodium hydride and methyl iodide to give chloride 210. Displacement of the chloride of 210 with various anilines, using Buchwald conditions, provided the compounds 211-214.

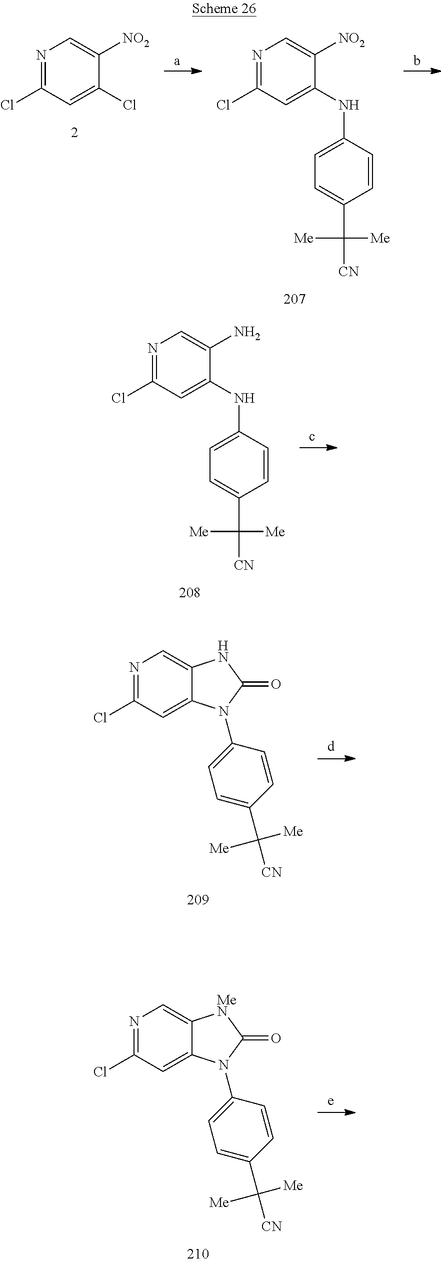 [SMILES: O=[N+]([O-])c1cnc(Cl)cc1Cl ; CC(C)(C#N)c1ccc(Nc2cc(Cl)ncc2[N+](=O)[O-])cc1 ; CC(C)(C#N)c1ccc(Nc2cc(Cl)ncc2N)cc1 ; CC(C)(C#N)c1ccc(-n2c(=O)[nH]c3cnc(Cl)cc32)cc1 ; Cn1c(=O)n(-c2ccc(C(C)(C)C#N)cc2)c2cc(Cl)ncc21]

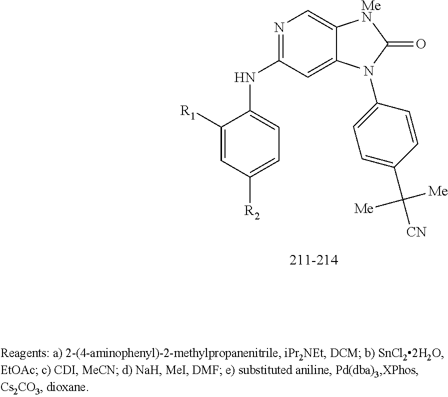

TABLE 7
Compounds 211-214

| SN | No. | Reagent | R1 | R2 |
|---|---|---|---|---|
| 39547 | 211 | 4-methoxy-2-methylaniline | 2-Me | 4-OMe |
| 39548 | 212 | 4-chloro-2-methylaniline | 2-Me | 4-Cl |
| 39546 | 213 | quinolin-6-amine | 3-CH═CHCH═N-4 | |
| 39549 | 214 | quinoxalin-6-amine | 3-N═CHCH═N-4 | |

Chloride 120 (Scheme 9) was displaced with a further set of anilines using modified Buchwald conditions to provide the compounds 215-226 (Scheme 27). PG-98, C₃

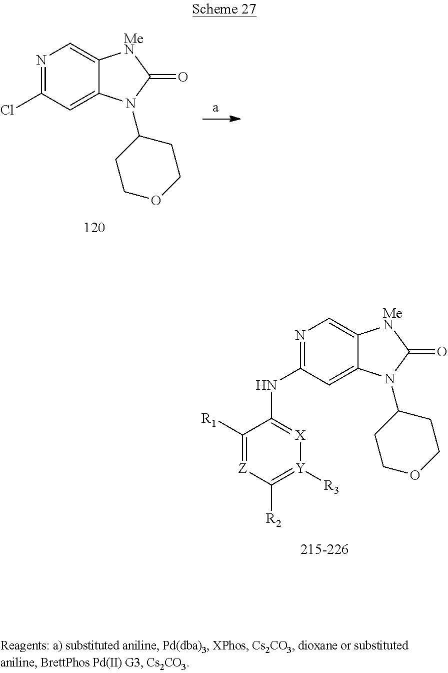

TABLE 8
Compounds 215-226

| SN | No. | Reagent | R1 | R2 | R3 | Aza |
|---|---|---|---|---|---|---|
| 40230 | 215 | 4-methoxyaniline | H | 4-OMe | H | — |
| 40284 | 216 | 4,5-dimethoxy-2- | 2- | 4-OMe | 5- | — |
| | | methylaniline | Me | | OMe | |
| 40249 | 217 | 5-chloro-3-methyl | 2- | 4-Cl | H | X |
| | | pyridin-2-amine | Me | | | |
| 40250 | 218 | 6-chloro-4- | 2- | 4-Cl | H | Y |
| | | methylpyridin-3- | Me | | | |
| | | amine | | | | |
| 40251 | 219 | 6-chloro-2- | 2- | 4-Cl | H | Z |
| | | methylpyridin-3- | Me | | | |
| | | amine | | | | |
| 40253 | 220 | 5-methoxy-3- | 2- | 4-OMe | H | X |
| | | methylpyridin-2- | Me | | | |
| | | amine | | | | |
| 40252 | 221 | 6-methoxy-2- | 2- | 4-OMe | H | Z |
| | | methylpyridin-3- | Me | | | |
| | | amine | | | | |
| 40262 | 222 | 2-methoxy-4- | 2- | 4-OMe | H | Y, Z |
| | | methylpyrimidin- | Me | | | |
| | | 5-amine | | | | |
| 40266 | 223 | 4-(6- | H | 4-(6- | H | — |
| | | methoxypyridin- | | methoxypyridin- | | |
| | | 3-yl)aniline | | 3-yl) | | |
| 40291 | 224 | 4-(2- | H | 4-(2- | H | — |
| | | methoxypyrimidin- | | methoxypyrimidin- | | |
| | | 5-yl)aniline | | 5-yl) | | |

| | | | | | |
|---|---|---|---|---|---|
| 40318 | 225 | 7- | 7- | 8a-N═CC═N-4a | — |
| | | methylquinoxalin- | Me | | |
| | | 6-amine | | | |
| 40258 | 226 | ethyl 6-amino-7- | 7- | 8a═NC(CO₂Et)═CH-4 | Y |
| | | methylimidazo | Me | | |
| | | [1,2-a]pyridine-2- | | | |
| | | carboxylate | | | |

Reaction of the imidazopyridinone 124 with di-tert-butyldicarbonate gave the carbamate 227 (Scheme 28). Hydrogenolysis of 227 gave the phenol 228. Alkylation of 228 with 2-chloro-N,N-dimethylethan-1-aminium chloride under basic conditions gave the ether 229 which was deprotected under acidic conditions to give imidazopyridinone 230 (SN40558).

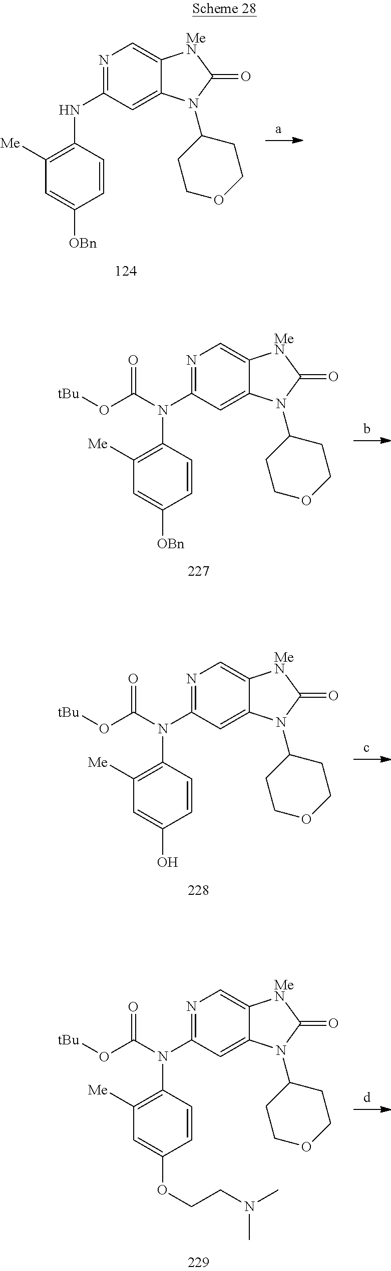 [SMILES: Cc1cc(OCc2ccccc2)ccc1Nc1cc2c(cn1)n(C)c(=O)n2C1CCOCC1 ; Cc1cc(OCc2ccccc2)ccc1N(C(=O)OC(C)(C)C)c1cc2c(cn1)n(C)c(=O)n2C1CCOCC1 ; Cc1cc(O)ccc1N(C(=O)OC(C)(C)C)c1cc2c(cn1)n(C)c(=O)n2C1CCOCC1 ; Cc1cc(OCCN(C)C)ccc1N(C(=O)OC(C)(C)C)c1cc2c(cn1)n(C)c(=O)n2C1CCOCC1]

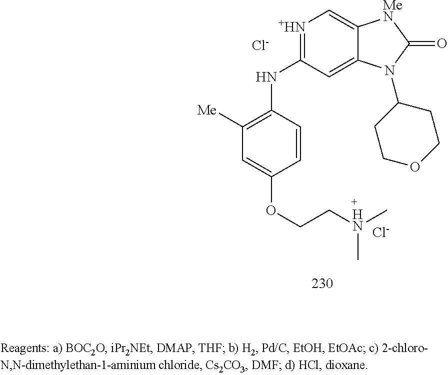

Reaction of the imidazopyridinone 51 with di-tert-butyldicarbonate gave the carbamate 231 (Scheme 29). Hydrogenolysis of 231 gave the phenol 232. Alkylation of 232 with 5-(bromomethyl)-1-methyl-2-nitro-1H-imidazole under basic conditions gave the ether 233 which was deprotected under acidic conditions to give prodrug 234 (SN39586).

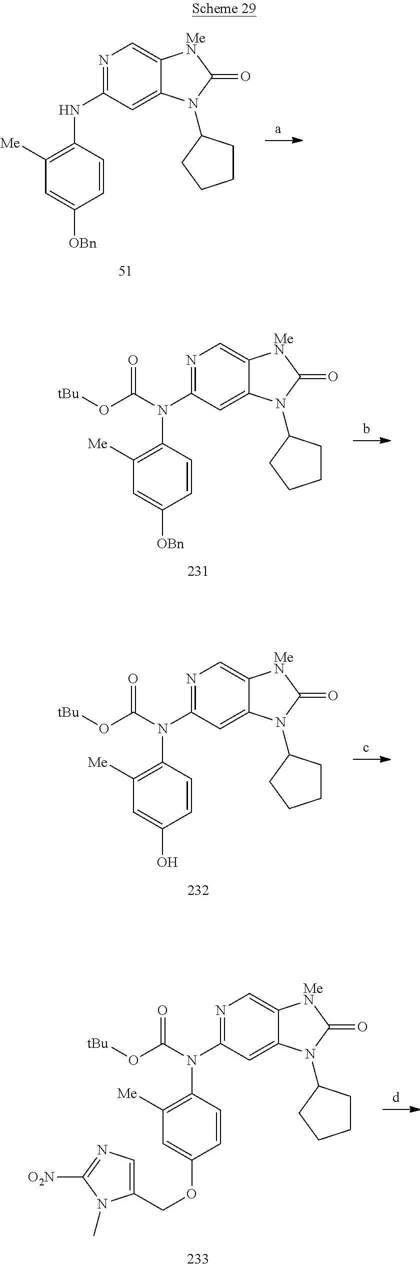 [SMILES: Cc1cc(OCc2ccccc2)ccc1Nc1cc2c(cn1)n(C)c(=O)n2C1CCCC1 ; Cc1cc(OCc2ccccc2)ccc1N(C(=O)OC(C)(C)C)c1cc2c(cn1)n(C)c(=O)n2C1CCCC1 ; Cc1cc(O)ccc1N(C(=O)OC(C)(C)C)c1cc2c(cn1)n(C)c(=O)n2C1CCCC1 ; Cc1cc(OCc2cnc([N+](=O)[O-])n2C)ccc1N(C(=O)OC(C)(C)C)c1cc2c(cn1)n(C)c(=O)n2C1CCCC1]

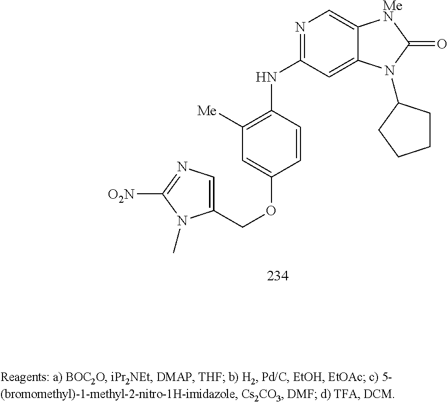 [SMILES: Cc1cc(OCc2cnc([N+](=O)[O-])n2C)ccc1Nc1cc2c(cn1)n(C)c(=O)n2C1CCCC1]

Phenol 228 (Scheme 28) was alkylated with 5-(chloromethyl)-1-methyl-2-nitro-1H-imidazole, 5-(1-chloroethyl)-1-methyl-2-nitro-1H-imidazole or (1-methyl-5-nitro-1H-imidazol-2-yl)methanol under basic conditions to give ethers 235, 237 and 238. Phenol 228 was also alkylated with (5-nitrothiophen-2-yl)methanol under Mitsunobu conditions to give ether 241. Esters 235, 237, 239 and 241 were deprotected under acidic conditions to give prodrugs 236, 238 and 240 (Scheme 30).

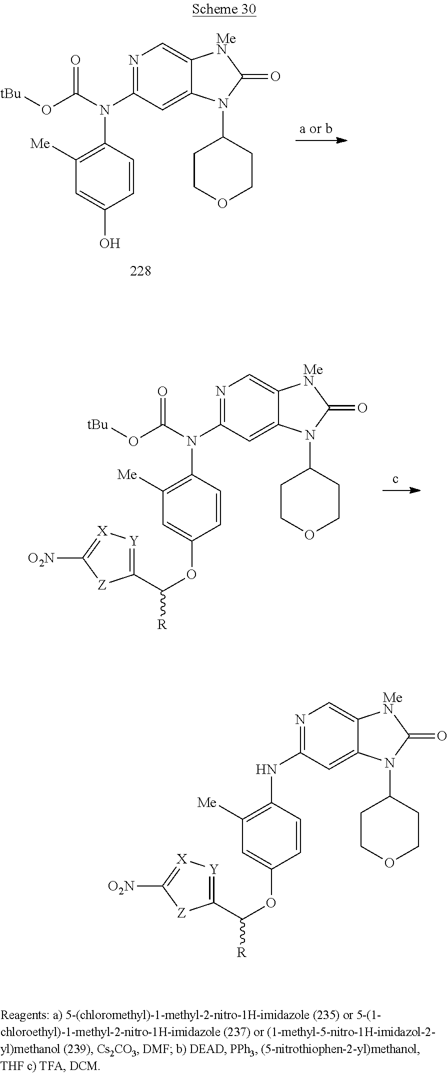

TABLE 9
Compounds 252-254

| SN | No. | X | Y | Z | R |
|---|---|---|---|---|---|
| 39897 | 236 | N | CH | NMe | H |
| 40458 | 238 | N | CH | NMe | Me |
| 40459 | 240 | CH | N | NMe | H |
| 40588 | 242 | CH | CH | S | H |

Reaction of the imidazopyridinone 199 with di-tert-butyldicarbonate gave the carbamate 243 (Scheme 31). Hydrogenolysis of 243 gave the phenol 244. Alkylation of 244 with 5-(chloromethyl)-1-methyl-2-nitro-1H-imidazole under basic conditions gave the ether 245 which was deprotected under acidic conditions to give prodrug 246 (SN39591).

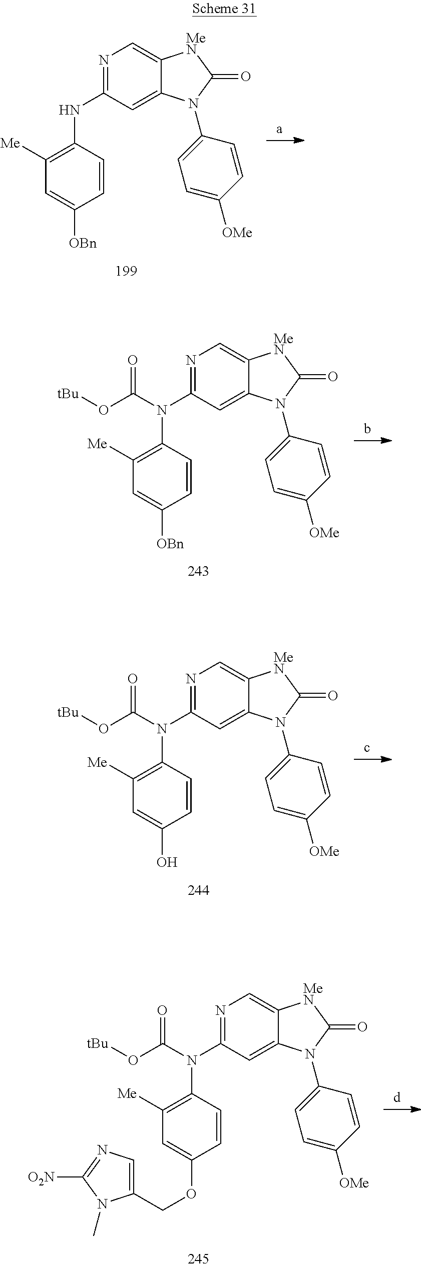 [SMILES: COc1ccc(-n2c(=O)n(C)c3cnc(Nc4ccc(OCc5ccccc5)cc4C)cc32)cc1 ; COc1ccc(-n2c(=O)n(C)c3cnc(N(C(=O)OC(C)(C)C)c4ccc(OCc5ccccc5)cc4C)cc32)cc1 ; COc1ccc(-n2c(=O)n(C)c3cnc(N(C(=O)OC(C)(C)C)c4ccc(O)cc4C)cc32)cc1 ; COc1ccc(-n2c(=O)n(C)c3cnc(N(C(=O)OC(C)(C)C)c4ccc(OCc5cnc([N+](=O)[O-])n5C)cc4C)cc32)cc1]

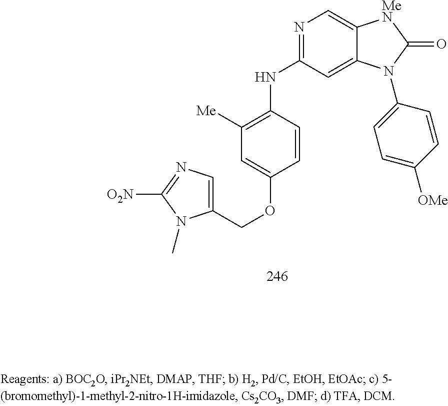 [SMILES: COc1ccc(-n2c(=O)n(C)c3cnc(Nc4ccc(OCc5cnc([N+](=O)[O-])n5C)cc4C)cc32)cc1]

Reaction of imidazopyridinone 48 with triphosgene and NaHCO₃ formed the intermediate carbamoyl chloride and subsequent reaction with 5-(hydroxymethyl)-1-methyl-2-nitro-1H-imidazole gave prodrug 247 (SN39725)(Scheme 32).

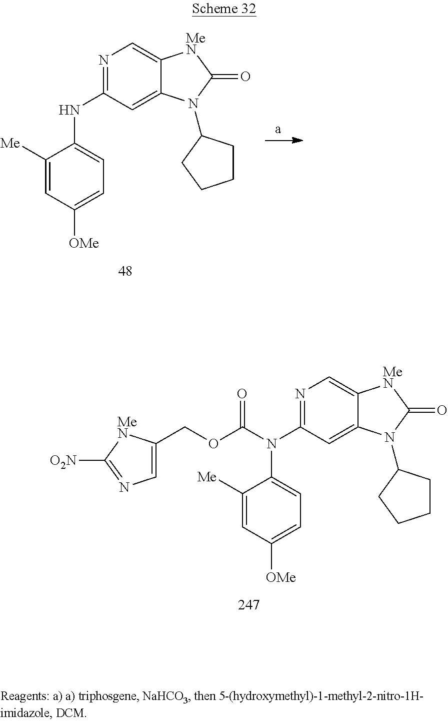 [SMILES: COc1ccc(Nc2cc3c(cn2)n(C)c(=O)n3C2CCCC2)c(C)c1 ; COc1ccc(N(C(=O)OCc2cnc([N+](=O)[O-])n2C)c2cc3c(cn2)n(C)c(=O)n3C2CCCC2)c(C)c1]

Reaction of imidazopyridinone 121 with triphosgene and NaHCO₃ formed the intermediate carbamoyl chloride and subsequent reaction with 5-(hydroxymethyl)-1-methyl-2-nitro-1H-imidazole, DMAP and K₂CO₃ gave prodrug 248 (SN39884)(Scheme 33). Optimisation of this procedure involved isolating intermediate carbamoyl chloride 249 before reaction with 1-(1-methyl-2-nitro-1H-imidazol-5-yl)ethan-1-ol or (1-methyl-5-nitro-1H-imidazol-2-yl)methanol to form prodrugs 250 (SN40425) and 251 (SN40353).

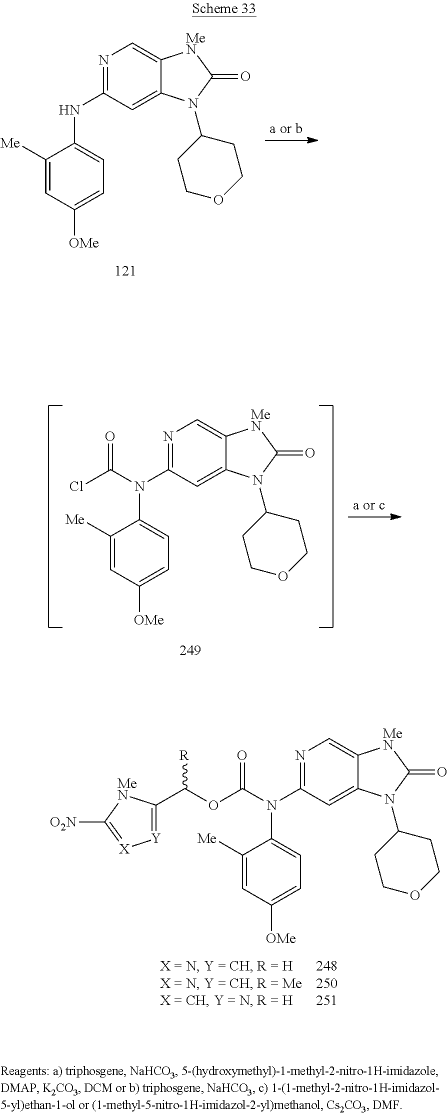

Reaction of imidazopyridinone 153 with (1-methyl-2-nitro-1H-imidazol-5-yl)methyl (4-nitrophenyl) carbonate gave prodrug 252 (SN40275)(Scheme 34).

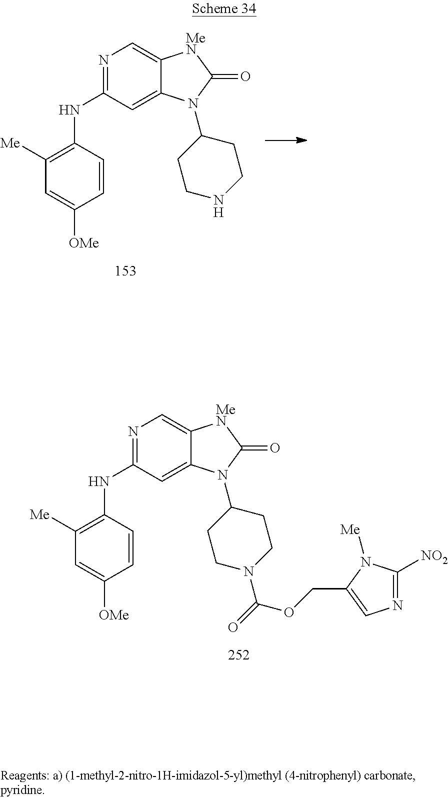 [SMILES: COc1ccc(Nc2cc3c(cn2)n(C)c(=O)n3C2CCNCC2)c(C)c1 ; COc1ccc(Nc2cc3c(cn2)n(C)c(=O)n3C2CCN(C(=O)OCc3cnc([N+](=O)[O-])n3C)CC2)c(C)c1]

Reaction of imidazopyridinone 185 with (1-methyl-2-nitro-1H-imidazol-5-yl)methyl (4-nitrophenyl) carbonate gave prodrug 253 (SN40302)(Scheme 35).

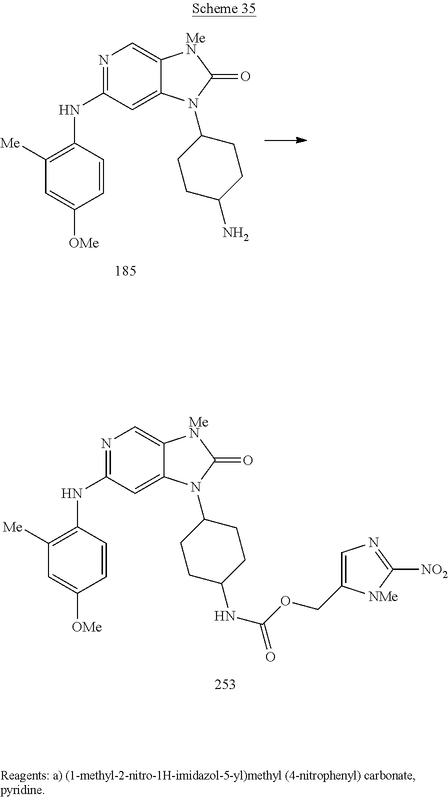 [SMILES: COc1ccc(Nc2cc3c(cn2)n(C)c(=O)n3C2CCC(N)CC2)c(C)c1 ; COc1ccc(Nc2cc3c(cn2)n(C)c(=O)n3C2CCC(NC(=O)OCc3cnc([N+](=O)[O-])n3C)CC2)c(C)c1]

Reaction of imidazopyridinones 129, 225 and 130 with triphosgene and NaHCO₃ formed the intermediate carbamoyl chloride and subsequent reaction with 5-(hydroxymethyl)-1-methyl-2-nitro-1H-imidazole and Cs₂CO₃ gave prodrugs 254-256 (Scheme 36).

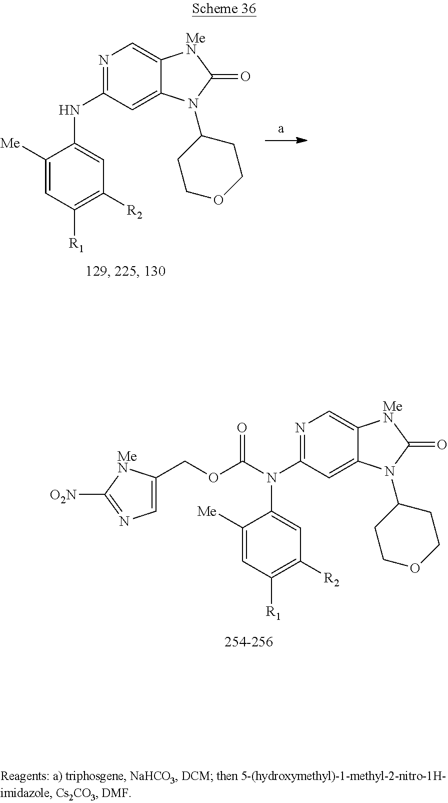

TABLE 10
Compounds 252-254

| SN | No. | R1 | R2 |
|---|---|---|---|
| 40527 | 254 | 8a═NCH═C(CO₂Et)-4 | |
| 40536 | 255 | 8a-N═CC═N-4a | |
| 40537 | 256 | 7a-OCH₂O-3a | |

Reaction of imidazopyridinones 122, 172 and 195 with triphosgene and NaHCO₃ formed the intermediate carbamoyl chloride and subsequent reaction with 5-(hydroxymethyl)-1-methyl-2-nitro-1H-imidazole and Cs₂CO₃ gave prodrugs 257-259 (Scheme 37).

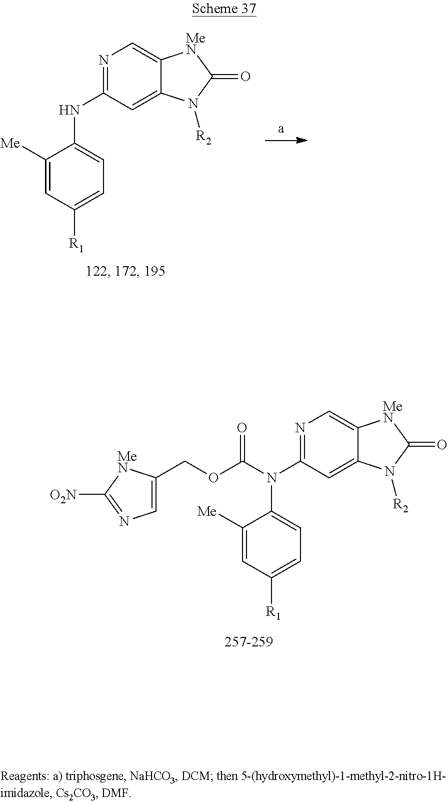

TABLE 11
Compounds 254-257

| SN | No. | R1 | R2 |
|---|---|---|---|
| 40316 | 257 | 4-Cl | CH(CH₂CH₂)₂O |
| 40317 | 258 | 4-OMe | CH(CH₂CH₂)₂CHOMe |
| 40427 | 259 | 4-OMe | p-C₆H₅OMe |

Imidazopyridione 230 was converted to a free base then reaction with 5-(bromomethyl)-1-methyl-4-nitro-1H-imidazole gave quaternary ammonium salt (SN40564) 260 (Scheme 38).

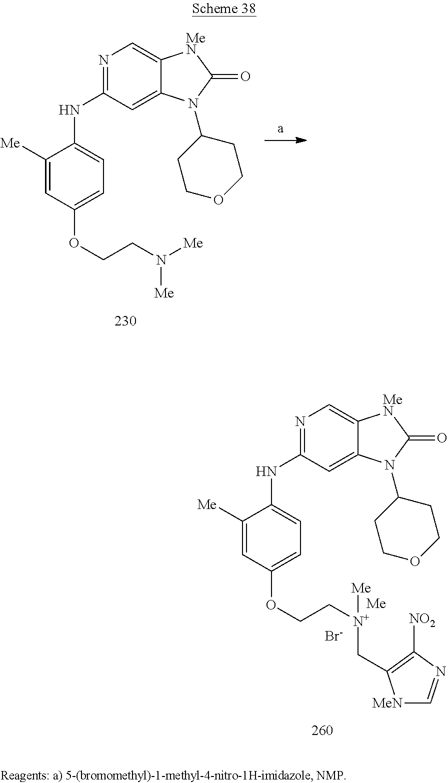 [SMILES: Cc1cc(OCCN(C)C)ccc1Nc1cc2c(cn1)n(C)c(=O)n2C1CCOCC1 ; Cc1cc(OCC[NH+](C)Cc2c([N+](=O)[O-])ncn2C)ccc1Nc1cc2c(cn1)n(C)c(=O)n2C1CCOCC1 ; [Br-]]

5.4 Uses of the Compounds of the Invention in Treating Cancer

The demonstrated DNA-PK inhibitory activity of the compounds of the invention makes them useful therapeutics against a range of diseases including cancer. In particular, the compounds of the invention are useful as anti-tumour agents. Without wishing to be bound by theory, the inventors believe that the DNA-PK inhibitor compounds described herein may act as anti-proliferative, apoptotic and/or anti-invasive agents in the treatment or prevention of solid and liquid tumours that are sensitive to the inhibition of DNA-PK, or mediated at least in part by DNA-PK.

Accordingly in one aspect the invention provides a method for treating a disease in which inhibition of DNA-PK is beneficial in a subject in need thereof, the method comprising administering to the subject a therapeutically effective amount of a compound of Formula I, II, III, IV, V, VI or VII or a pharmaceutically acceptable salt thereof.

In another aspect the invention provides a use of a compound of Formula I, II, III, IV, V, VI or VII, or a pharmaceutically acceptable salt thereof, in the manufacture of a medicament for the treatment of a disease in which inhibition of DNA-PK is beneficial.

In another aspect the invention provides a compound of Formula I, II, III, IV, V, VI or VII, or a pharmaceutically acceptable salt thereof, for use in the treatment of a disease in which inhibition of DNA-PK is beneficial.

In one embodiment, the disease is cancer.

In one embodiment the cancer is a solid tumour including, but not limited to, carcinoma, sarcoma, leukaemia and lymphoid malignancy.

In one embodiment the cancer is selected from the group consisting of haematologic malignancies including leukaemia (including chronic lymphocytic leukaemia, acute lymphoctic leukaemia, chronic myelogenous leukaemia, multiple myeloma) lymphomas such as Hodgkin's disease, non-Hodgkin's lymphomas (including mantle cell lymphoma), and myelodysplastic syndromes, and also solid tumours and their metastases such as breast cancer, lung cancer (non-small small cell lung cancer (NSCLC), small cell lung cancer (SCLC), squamous cell carcinoma), endometrial cancer, tumours of the central nervous system such as gliomas, dysembryoplastic neuroepithelial tumour, glioblastoma multiforme, mixed glioms, medulloblastoma, retinoblastoma, neuroblastoma, germinoma and teratoma, cancers of the gastrointestinal tract such as gastric cancer, oesophageal cancer, hepatocellular (liver) carcinoma, cholangiocarcinomas, colon and rectal carcinomas, cancers of the small intestine, pancreatic, cancers of the skin such as melanomas (in particular metastatic melanoma), thyroid cancers, cancers of the head and neck and cancers of the salivary glands, bile duct, bone, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma Ewing's sarcoma, gastrointestinal stromal tumour (GIST), Kaposi's sarcoma, and paediatric cancers such as rhabdomyosarcomas and neuroblastomas.

In one embodiment the cancer is a tumour that includes significant hypoxic fractions.

In one embodiment, the cancer is selected from squamous cell carcinoma (including head and neck squamous cell carcinoma (HNSCC) and non-small cell lung cancer (NSCLC)), pancreatic ductal adenocarcinoma, cervical and prostate cancer.

In the methods of the invention, a therapeutically effective amount of a compound of Formula I, II, III, IV, V, VI or VII or a pharmaceutically acceptable salt or solvate thereof, is administered to a subject in need thereof.

A therapeutically effective amount may cause any of the changes observable or measurable in a subject as described in the definition of “therapy”, “treatment” and “prophylaxis” above.

For example, in the treatment of cancer, a therapeutically effective amount of a compound of the invention may reduce the number of cancer or tumour cells; reduce the overall tumour size; inhibit or stop tumour cell infiltration into peripheral organs including, for example, the soft tissue and bone; inhibit and stop tumour metastasis; inhibit and stop tumour growth; relieve to some extent one or more of the symptoms associated with the cancer; reduce morbidity and mortality; improve quality of life; or a combination of such effects.

The efficacy of the treatment can be measured by assessing the duration of survival, time to disease progression (TTP), the response rates (RR), duration of response, and/or quality of life.

Therapeutically effective amounts may vary depending on route of administration, excipient usage, and co-usage with other agents. For example, where a combination therapy is used, the amount of the compound of the invention or pharmaceutically acceptable salt described in this specification and the amount of the other pharmaceutically active agent(s) are, when combined, jointly effective to treat the targeted disease in the subject.

Anti-cancer effects which are accordingly useful in the treatment of cancer in a subject include, but are not limited to anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. Anti-cancer effects include prophylactic treatment as well as treatment of existing disease.

In addition to administration of a compound of the invention, the methods of treatment of the invention may include other therapy including, but not limited to, radiotherapy and/or chemotherapy.

Radiotherapy may include one or more of the following categories of therapy:
(a) External radiation therapy using electromagnetic radiation, and intraoperative radiation therapy using electromagnetic radiation;
(b) Internal radiation therapy or brachytherapy; including interstitial radiation therapy or intraluminal radiation therapy; and
(c) Systemic radiation therapy, including but not limited to iodine 131 and strontium 89.

Modern radiotherapy is typically delivered by linear accelerators that generate high energy X-rays that can be collimated to shape the treatment field. Intensity modulated radiation therapy (IMRT) uses non-uniform computer-controlled radiation fields to optimise delivery to the tumour tissue rather than surrounding normal tissue. Standard fractionated radiotherapy (FRT) is typically delivered with small (1.8-2.0 Gy) fractions over 4-7 weeks for a total dose of 30-70 Gy. Improvements in treatment planning and delivery have allowed the delivery of hypo-fractionated radiotherapy where a small number of high (15-20 Gy) doses can be delivered to tumours. This is known as stereotactic body radiation therapy (SBRT) or stereotactic ablative brain radiation (SABR). High energy charged particles such as protons and carbon ions may also be used to treat tumours and have the advantage of delivering most of the particle energy within the tumour. Brachytherapy uses radioactive implants to deliver radiation therapy internally to patients and is sometimes called intra-operative radiation therapy (IORT).

Chemotherapy may include one or more of the following categories of anti-tumour substances:
(a) Antineoplastic agents and combinations thereof, such as DNA alkylating agents (for example cisplatin, oxaliplatin, carboplatin, cyclophosphamide, nitrogen mustards like ifosfamide, bendamustine, melphalan, chlorambucil, busulphan, temozolamide and nitrosoureas like carmustine); antimetabolites (for example gemcitabine and antifolates such as fluoropyrimidines like 5-fluorouracil and tegafur, raltitrexed, methotrexate, cytosine arabinoside, and hydroxyurea); anti-tumour antibiotics (for example anthracyclines like adriamycin, bleomycin, doxorubicin, liposomal doxorubicin, pirarubicin, daunomycin, valrubicin, epirubicin, idarubicin, mitomycin-C, dactinomycin, amrubicin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, irinotecan, topotecan and camptothecin); inhibitors of DNA repair mechanisms such as CHK kinase; ATM inhibitors (such as AZD0156 and AZD1390); inhibitors of poly (ADP-ribose) polymerase (PARP inhibitors, including olaparib); and Hsp90 inhibitors such as tanespimycin and retaspimycin, inhibitors of ATR kinase (such as AZD6738); and inhibitors of WEE1 kinase (such as AZD1775/MK-1775); and
(b) Immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor; approaches to decrease T-cell anergy or regulatory T-cell function; approaches that enhance T-cell responses to tumours, such as blocking antibodies to CTLA4 (for example ipilimumab and tremelimumab), B7H1, PD-1 (for example BMS-936558 or AMP-514), PD-L1 (for example MEDI4736 (durvalumab)) and agonist antibodies to CD 137; approaches using transfected immune cells such as cytokine-transfected dendritic cells; approaches using cytokine-transfected tumour cell lines, approaches using antibodies to tumour associated antigens, and antibodies that deplete target cell types (e.g., unconjugated anti-CD20 antibodies such as Rituximab, radiolabeled anti-CD20 antibodies Bexxar and Zevalin, and anti-CD54 antibody Campath); approaches using anti-idiotypic antibodies; approaches that enhance Natural Killer cell function; and approaches that utilize antibody-toxin conjugates (e.g. anti-CD33 antibody Mylotarg); immuno toxins such as moxetumumab pasudotox; agonists of toll-like receptor 7 or toll-like receptor 9.

In one embodiment, the invention provides a method for treating cancer, the method comprising administering to the subject a therapeutically effective amount of a compound of Formula I, II, III, IV, V, VI or VII, or a pharmaceutically acceptable salt or solvate thereof, in combination with radiotherapy, wherein the compound of Formula I, II, III, IV, V, VI or VII, or a pharmaceutically acceptable salt or solvate thereof, is administered simultaneously, separately or sequentially with the radiotherapy.

In one embodiment, radiotherapy is administered to the subject before, during or after administration of the compound of Formula I, II, III, IV, V, VI or VII or pharmaceutically acceptable salt or solvate thereof.

In one embodiment, the invention provides a use of a compound of Formula I, II, III, IV, V, VI or VII, or a pharmaceutically acceptable salt or solvate thereof, in the manufacture of a medicament for the treatment of cancer.

In one embodiment, the medicament is for simultaneous, separate or sequential administration with radiotherapy.

In one embodiment the radiotherapy is selected from the group consisting of IMRT, FRT, SBRT, SABR and IORT.

In one embodiment, the invention provides a method for treating cancer, the method comprising administering to the subject a therapeutically effective amount of a compound of Formula I, II, III, IV, V, VI or VII, or a pharmaceutically acceptable salt or solvate thereof, in combination with chemotherapy, wherein the compound of Formula I, II, III, IV, V, VI or VII, or a pharmaceutically acceptable salt or solvate thereof, is administered simultaneously, separately or sequentially with the chemotherapy.

In one embodiment, chemotherapy is administered to the subject before, during or after administration of the compound of Formula I, II, III, IV, V, VI or VII or pharmaceutically acceptable salt or solvate thereof.

In one embodiment, the invention provides a use of a compound of Formula I, II, III, IV, V, VI or VII, or a pharmaceutically acceptable salt or solvate thereof, in the manufacture of a medicament for the treatment of cancer.

In one embodiment, the medicament is for simultaneous, separate or sequential administration with chemotherapy.

The compound of Formula I, II, III, IV, V, VI or VII or pharmaceutically acceptable salt or solvate thereof, will normally be administered to the subject at a unit dose within the range 2.5-5000 mg/m² body area of the animal, or approximately 0.05-100 mg/kg. A unit dose form such as a tablet or capsule will usually contain, for example 0.1-250 mg of active agent. The dosage to be administered will necessarily be varied depending upon the subject to be treated, the particular route of administration, any co-administered therapies, and the severity of the disease being treated. The optimum dosage will be determined by the practitioner who is treating the subject.

The compounds of the invention may be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally, or vaginally (for example, as a pessary, cream, or foam); parenterally (including intramusclularly, intravenously, subcutaneously, or intrathecally as, for example, a sterile solution or suspension); nasally; intraperitoneally; subcutaneously; transdermally (for example as a patch applied to the skin); or topically (for example, as a cream, ointment or spray applied to the skin). At least one compound and/or salt as described herein may also be formulated for inhalation.

In another aspect the invention provides a pharmaceutical composition comprising a compound of Formula I, II, III, IV, V, VI or VII or a pharmaceutically acceptable salt or solvate thereof, in combination with one or more pharmaceutically acceptable excipients.

The pharmaceutical composition of the invention may be formulated to be administered orally, topically, parenterally, by inhalation or spray or rectally in dosage unit formulations. The term ‘administration by injection’ includes intravenous, intramuscular, subcutaneous and parenteral injections, as well as use of infusion techniques. One or more compounds may be present in association with one or more non-toxic pharmaceutically acceptable carriers and if desired other active ingredients.

A “pharmaceutically acceptable carrier” is a pharmaceutically acceptable material, composition or vehicle, such as a liquid, diluent, excipient, filler, solvent or encapsulating material involved in transporting the subject compound around the body. Each carrier is “acceptable” in that it is compatible with the other ingredients of the formulation and is not harmful to the subject. The pharmaceutically acceptable compositions of the invention may also include other active agents providing additional therapeutic functions.

Examples of materials that may serve as pharmaceutically acceptable carriers include but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. See Remington: The Science and Practice of Pharmacy, 20th ed. (Alfonso R. Gennaro ed.), 2000.

Pharmaceutical compositions intended for oral use may be prepared according to any suitable method known to the art. Such compositions may contain one or more agents selected from the group consisting of diluents, sweetening agents, flavouring agents, colouring agents and preserving agents in order to provide palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed. These compounds may also be prepared in solid, rapidly released form.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally occurring phosphatide, for example, lecithin, or condensation products or an alkylene oxide with fatty acids, for example polyoxyethylene stearate, or condensation products of ethylene oxide with long chain aliphatic alcohols, for example heptadecaethylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more colouring agents, one or more flavouring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavouring and colouring agents, may also be present.

The pharmaceutical compositions of the invention may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions.

The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Pharmaceutical compositions as described herein for rectal, vaginal, or urethral administration may be presented as a suppository, which may be prepared by mixing one or more compounds or salts as described herein with one or more suitable non-irritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

The pharmaceutical compositions described herein may be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required. The ointments, pastes, creams, and gels may comprise excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a compound as described herein, excipients such as lactose, talc, silicic acid, aluminum hydroxide, calcium silicates, and polyamide powder, or mixtures of these substances. Sprays may additionally contain customary propellants, such as chlorofluorohydrocarbons and volatile unsubstituted hydrocarbons, such as butane and propane.

Transdermal patches have the added advantage of providing controlled delivery to the body. Such dosage forms may be made by dissolving or dispersing the compound in the proper medium. Absorption enhancers may also be used to increase the flux across the skin. The rate of such flux may be controlled by either providing a rate controlling membrane or dispersing the compound in a polymer matrix or gel. Ophthalmic formulations, eye ointments, powders, solutions, and the like, may also comprise at least one of the compounds or salts as described herein.

The pharmaceutical compositions as described herein that are suitable for parenteral administration comprise at least one compound of the invention or a pharmaceutically acceptable salt thereof, in combination with one or more pharmaceutically acceptable sterile isotonic aqueous or nonaqueous solutions, dispersions, suspensions, or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, chelators and the like.

In some embodiments, isotonic agents, such as sugars, sodium chloride, and the like may be included into the compositions. In addition, prolonged absorption of the injectable pharmaceutical form may be brought about by the inclusion of agents that delay absorption such as aluminum mono stearate and gelatin.

### 6. EXAMPLES

The following examples are representative of the invention and the detailed methods for preparing these compounds; however, the scope of the invention is not limited to these examples.

All final products were analysed by reverse-phase HPLC, (ZORBAX Eclipse XDB C8 5 μm column, 4.6×150 mm; Agilent Technologies) using an Agilent Technologies 1260 Infinity equipped with a diode-array detector. Mobile phases were gradients of 80% acetonitrile/20% H₂O (v/v) in 45 mM ammonium formate at pH 3.5 and 0.8 mL/min. Final compound purity was determined by monitoring at 330±50 nM and was >95%. Melting points were determined on an Electrothermal 2300 Melting Point Apparatus. NMR spectra were obtained on a Bruker Avance 400 spectrometer at 400 MHz for 1H spectra. Chemical shifts and coupling constants were recorded in units of ppm and Hz, respectively. Low resolution mass spectra were gathered by direct injection of methanolic solutions into an Agilent 6120 mass spectrometer using an atmospheric pressure chemical ionization (APCI) mode with a fragmentor voltage of 50 V and a drying gas temperature of 250° C. High resolution mass spectra (HRMS) were measured on an Agilent Technologies 6530 Accurate-Mass Quadrupole Time of Flight (Q-TOF) LC/MS interfaced with an Agilent Jet Stream Electrospray Ionization (ESI) source allowing positive or negative ions detection. Organic solutions were dried over MgSO₄ or Na₂SO₄ and solvents were evaporated under reduced pressure on a rotary evaporator. Thin-layer chromatography was carried out on aluminium-backed silica gel plates (Merck 60 F₂₅₄) with visualization of components by UV light (254 nm) or exposure to I₂. Column chromatography was carried out on silica gel (Merck 230-400 mesh). BrettPhos G3 refers to [(2-di-cyclohexylphosphino-3,6-dimethoxy-2′, 4′, 6′-triisopropyl-1,1′-biphenyl)-2-(2′-amino-1,1′-biphenyl)]palladium(II) methanesulfonate methanesulfonate, CDI refers to carbonyldiimidazole, Cs₂CO₃ refers to caesium carbonate, DCM refers to dichloromethane, DIPEA refers to diisopropylethylamine, DMAP refers to 4-dimethylaminopyridine, DMF refers to dimethylformamide, DMSO refers to dimethyl sulfoxide, EtOAc refers to ethyl acetate, EtOH refers to ethanol, MeOH refers to methanol, MeCN refers to acetonitrile, MgSO₄ refers to magnesium sulfate, NMP refers to N-methylpyrrolidone, Pd₂dba₃ refers to tris(dibenzylideneacetone)dipalladium(0), pet. ether refers to petroleum ether boiling fraction 40-60° C., THF refers to tetrahydrofuran, XPhos refers to 2-dicyclohexylphosphino-2′, 4′, 6′-triisopropylbiphenyl.

### Example 1: SN39228 1-Cyclopentyl-3-methyl-6-(phenylamino)-1,3-dihydro-2H-imidazo[4,5-c]pyridin-2-one (7)

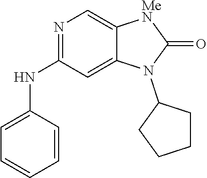 [SMILES: Cn1c(=O)n(C2CCCC2)c2cc(Nc3ccccc3)ncc21]

2,4-Dichloro-5-nitropyridine (2). A mixture of 4-chloro-5-nitropyridin-2-ol (1) (5.38 g, 30.8 mmol), POCl₃ (60 mL) and tetramethylammonium chloride (10.1 g, 32.5 mmol) was stirred at 120° C. for 3 h. The mixture was cooled, poured into ice/water (500 mL) and stirred for 1 h at 0-10° C. The mixture was extracted with DCM (3×100 mL) and the combined organic extract dried (MgSO₄). The solution was filtered through a short column of neutral alumina, washing with DCM (50 mL). The solvent was evaporated to give nitropyridine 2 as a clear oil (5.43 g, 91%): ¹H NMR (CDCl₃) δ 8.97 (s, 1H, H-6), 7.59 (s, 1H, H-3); MS m/z 192.9 (MH⁺, 100%), 194.9 (MH⁺, 70%).

2-Chloro-N-cyclopentyl-5-nitropyridin-4-amine (3). A solution of cyclopentylamine (0.95 mL, 9.6 mmol) in dry DCM (5 mL) was added dropwise to a stirred solution of nitropyridine 2 (1.76 g, 9.12 mmol) and iPr₂NEt (1.87 mL, 10.94 mmol) in dry DCM (50 mL) at 5° C. The mixture was stirred at 20° C. for 16 h before being diluted with DCM (100 mL), washed with water (3×50 mL), dried (MgSO₄) and the solvent evaporated. The residue was purified by chromatography, eluting with 10% EtOAc/pet. ether, to give amine 3 (2.25 g, 100%) as a yellow oil: ¹H NMR (CDCl₃) δ 9.01 (s, 1H, H-6), 8.17 (br s, 1H, 4-NH), 6.76 (s, 1H, H-3), 3.93 (br dpent, J=6.7, 5.3 Hz, 1H, NCH), 2.10-2.18 (m, 2H, CH₂), 1.70-1.88 (m, 4H, 2×CH₂), 1.59-1.69 (m, 2H, CH₂); MS m/z 242.1 (MH⁺, 100%), 244.1 (MH⁺, 35%).

6-Chloro-N⁴-cyclopentylpyridine-3,4-diamine (4). A solution of nitropyridine 3 (1.92 g, 7.93 mmol) in EtOAc (20 ml) was added dropwise to a stirred suspension of SnCl₂·2H₂O (7.16 g, 31.7 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The precipitate was filtered and washed with EtOAc (100 mL). The combined organic fraction was dried (MgSO₄), filtered and the solvent evaporated to give diamine 4 as a white powder: mp 103-105° C.; ¹H NMR [(CD₃)₂SO] δ 7.35 (s, 1H, H-2), 6.29 (s, 1H, H-5), 5.48 (d, J=6.2 Hz, 1H, 4-NH), 4.78 (br s, 2H, 3NH₂), 3.75 (br dpent, J=6.7, 5.3 Hz, 1H, NCH), 1.90-1.98 (m, 2H, CH₂), 1.62-1.72 (m, 2H, CH₂), 1.42-1.60 (m, 4H, 2×CH₂); MS m/z 212.2 (MH⁺, 100%), 214.2 (MH⁺, 35%). Anal calcd for C₁₀H₁₄ClN₃·0.1 EtOAc: C, 56.65; H, 6.77; N, 19.06. Found: C, 56.66; H, 6.84; N, 19.20%.

6-Chloro-1-cyclopentyl-1,3-dihydro-2H-imidazo[4,5-c]pyridin-2-one (5). CDI (1.36 g, 8.38 mmol) was added to a stirred solution of diamine 4 (1.69 g, 7.98 mmol) in dry MeCN (80 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was precipitated from 50% EtOAc/pet. ether, to give the pyridinone 5 (1.80 g, 95%) as a white powder: mp 222-224° C.; ¹H NMR (CDCl₃) δ 9.76 (br s, 1H, 3-NH), 8.13 (s, 1H, H-4), 7.01 (s, 1H, H-7), 4.79 (pent, J=8.7 Hz, 1H, 1-CH), 1.94-2.12 (m, 6H, 3×CH₂), 1.72-1.81 (m, 2H, CH₂); MS m/z 238.2 (MH⁺, 100%), 240.2 (MH⁺, 35%). Anal calcd for C₁₁H₁₂ClN₃O: C, 55.59; H, 5.09; N, 17.68. Found: C, 55.31; H, 5.16; N, 17.61%.

6-Chloro-1-cyclopentyl-3-methyl-1,3-dihydro-2H-imidazo[4,5-c]pyridin-2-one (6). NaH (60% dispersion, 312 mg, 7.8 mmol) was added to a stirred solution of pyridinone 5 (1.68 g, 7.1 mmol) and MeI (0.57 mL, 9.2 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (40-50%) of EtOAc/pet. ether, to give chloride 6 (1.49 g, 83%) as white crystals: mp 141-142° C.; ¹H NMR (CDCl₃) δ 7.98 (s, 1H, H-4), 6.98 (s, 1H, H-7), 4.81 (pent, J=8.8 Hz, 1H, 1-CH), 3.44 (s, 3H, 3-CH₃), 1.92-2.07 (m, 6H, 3×CH₂), 1.69-1.79 (m, 2H, CH₂); MS m/z 252.2 (MH⁺, 100%), 254.2 (MH⁺, 35%). Anal calcd for C₁₂H₁₄ClN₃O: C, 56.26; H, 5.61; N, 16.69. Found: C, 57.26; H, 5.68; N, 16.86%.

1-Cyclopentyl-3-methyl-6-(phenylamino)-1,3-dihydro-2H-imidazo[4,5-c]pyridin-2-one (7). A degassed mixture of chloride 6 (120 mg, 0.48 mmol), aniline (53 mg, 0.57 mmol), Pd₂dba₃ (22 mg, 24 μmol), XPhos (46 mg, 96 μmol) and Cs₂CO₃ (313 mg, 0.96 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with a gradient (40-60%) of EtOAc/pet. ether, to give imidazopyridinone 7 (84 mg, 57%) as a tan powder: mp (EtOAc/pet ether) 168-170° C.; ¹H NMR (CDCl₃) δ 7.83 (s, 1H, H-4), 7.33 (br dd, J=8.6, 7.2 Hz, 2H, H-3′, H-5′), 7.26 (br d, J=8.6 Hz, 2 H, H-2′, H-6′), 7.01 (tt, J=7.2, 1.2 Hz, 1H, H-4′), 6.64 (s, 1H, H-7), 6.48 (s, 1H, 6-NH), 4.77 (pent, J=8.8 Hz, 1H, 1-CH), 3.40 (s, 3H, 3-CH₃), 1.95-2.05 (m, 4H, 2×CH₂), 1.81-1.90 (m, 2H, CH₂), 1.63-1.72 (m, 2H, CH₂); MS m/z 309.2 (MH⁺, 100%). Anal calcd for C₁₈H₂₀N₄O: C, 70.11; H, 6.54; N, 18.17. Found: C, 70.06; H, 6.64; N, 18.23%. HPLC purity 100.0% Example 2: SN39229 1-Cyclopentyl-3-methyl-6-(4-methylphenylamino)-1,3-dihydro-2H-imidazo[4,5-c]pyridin-2-one (8).

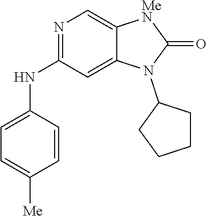 [SMILES: Cc1ccc(Nc2cc3c(cn2)n(C)c(=O)n3C2CCCC2)cc1]

A degassed mixture of chloride 6 (120 mg, 0.48 mmol), 4-methylaniline (61 mg, 0.57 mmol), Pd₂dba₃ (22 mg, 24 μmol), XPhos (46 mg, 96 μmol) and Cs₂CO₃ (313 mg, 0.96 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (40-60%) of EtOAc/pet. ether, to give imidazopyridinone 8 (99 mg, 64%) as a tan powder: mp (EtOAc/pet ether) 153-154° C.; ¹H NMR (CDCl₃) δ 7.80 (s, 1H, H-4), 7.12-7.17 (m, 4H, H-2′, H-3′, H-5′, H-6′), 6.58 (d, J=0.6 Hz, 1H, H-7), 6.39 (s, 1H, 6-NH), 4.75 (pent, J=8.8 Hz, 1 H, 1-CH), 3.38 (s, 3H, 3-CH₃), 2.33 (s, 3H, 4′-CH₃), 1.95-2.03 (m, 4H, 2×CH₂), 1.82-1.90 (m, 2H, CH₂), 1.66-1.72 (m, 2H, CH₂). Anal calcd for C₁₉H₂₂N₄O: C, 70.78; H, 6.88; N, 17.38. Found: C, 70.39; H, 7.21; N, 17.43%. HPLC purity 98.5%.

### Example 3: SN39231 1-Cyclopentyl-3-methyl-6-(3-methylphenylamino)-1,3-dihydro-2H-imidazo[4,5-c]pyridin-2-one (9)

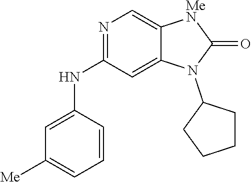 [SMILES: Cc1cccc(Nc2cc3c(cn2)n(C)c(=O)n3C2CCCC2)c1]

A degassed mixture of chloride 6 (120 mg, 0.48 mmol), 3-methylaniline (61 mg, 0.57 mmol), Pd₂dba₃ (22 mg, 24 μmol), XPhos (46 mg, 96 μmol) and Cs₂CO₃ (311 mg, 0.96 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-70%) of EtOAc/pet. ether, to give imidazopyridinone 9 (131 mg, 85%) as a tan powder: mp (EtOAc/pet ether) 124-126° C.; ¹H NMR (CDCl₃) δ 7.82 (d, J=0.6 Hz, 1H, H-4), 7.21 (br t, J=7.7 Hz, 1H, H-5′), 7.03-7.10 (m, 2H, H-2′, H-6′), 6.85 (d, J=7.5 Hz, 1H, H-4′), 6.68 (d, J=0.6 Hz, 1H, H-7), 6.44 (s, 1H, 6-NH), 4.80 (pent, J=8.8 Hz, 1H, 1-CH), 3.97 (s, 3H, 3-CH₃), 2.34 (s, 3H, 3′-CH₃), 1.94-2.05 (m, 4H, 2×CH₂), 1.82-1.90 (m, 2H, CH₂), 1.66-1.74 (m, 2H, CH₂). Anal calcd for C₁₉H₂₂N₄O: C, 70.78; H, 6.88; N, 17.38. Found: C, 70.50; H, 6.99; N, 17.56%. HPLC purity 99.7%

### Example 4: SN39232 1-Cyclopentyl-3-methyl-6-(2-methylphenylamino)-1,3-dihydro-2H-imidazo[4,5-c]pyridin-2-one (10)

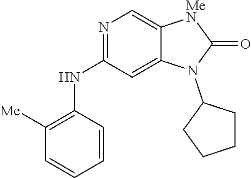 [SMILES: Cc1ccccc1Nc1cc2c(cn1)n(C)c(=O)n2C1CCCC1]

A degassed mixture of chloride 6 (120 mg, 0.48 mmol), 2-methylaniline (61 mg, 0.57 mmol), Pd₂dba₃ (22 mg, 24 μmol), XPhos (46 mg, 96 μmol) and Cs₂CO₃ (311 mg, 0.96 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-70%) of EtOAc/pet. ether, to give imidazopyridinone 10 (131 mg, 85%) as a tan foam: ¹H NMR (CDCl₃) δ 7.81 (d, J=0.6 Hz, 1H, H-4), 7.39 (d, J=7.6 Hz, 1H, H-3′), 7.24 (d, J=7.4 Hz, 1 H, H-6′), 7.20 (br t, J=7.7 Hz, 1H, H-4′), 7.02 (dt, J=7.4, 1.1 Hz, 1H, H-5′), 6.45 (d, J=0.6 Hz, 1H, H-7), 6.13 (s, 1H, 6-NH), 4.73 (pent, J=8.8 Hz, 1H, 1-CH), 3.39 (s, 3H, 3-CH₃), 2.29 (s, 3H, 3′-CH₃), 1.92-2.02 (m, 4H, 2×CH₂), 1.74-1.84 (m, 2H, CH₂), 1.62-1.70 (m, 2H, CH₂); MS m/z 323.2 (MH⁺, 100%). Anal calcd for C₁₉H₂₂N₄O·¼ EtOAc: C, 70.35; H, 6.94; N, 16.92. Found: C, 70.34; H, 7.10; N, 16.77%. HPLC purity 97.7%.

### Example 5: SN39236 1-Cyclopentyl-6-((4-methoxyphenyl)amino)-3-methyl-1,3-dihydro-2H-imidazo[4,5-c]pyridin-2-one (11)

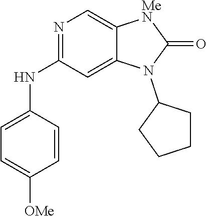 [SMILES: COc1ccc(Nc2cc3c(cn2)n(C)c(=O)n3C2CCCC2)cc1]

A degassed mixture of chloride 6 (128 mg, 0.51 mmol), 4-methoxyaniline (75 mg, 0.61 mmol), Pd₂dba₃ (23 mg, 25 μmol), XPhos (48 mg, 102 μmol) and Cs₂CO₃ (331 mg, 1.02 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 11 (72 mg, 42%) as a brown powder: mp (EtOAc/pet ether) 159-161° C.; ¹H NMR (CDCl₃) δ 7.77 (s, 1H, H-4), 7.21 (ddd, J=8.9, 3.5, 2.2 Hz, 2 H, H-2′, H-6′), 6.91 (ddd, J=8.9, 3.5, 2.2 Hz, 2 H, H-3′, H-5′), 6.42 (s, 1H, H-7), 6.27 (br s, 1H, 6-NH), 4.72 (pent, J=8.8 Hz, 1H, 1-CH), 3.82 (s, 3H, 4′-OCH₃), 3.38 (s, 3H, 3-CH₃), 1.93-2.02 (m, 4H, 2×CH₂), 1.78-1.88 (m, 2H, CH₂), 1.63-1.72 (m, 2H, CH₂); MS m/z 339.2 (MH⁺, 100%). Anal calcd for C₁₉H₂₂N₄O₂·¼EtOAc: C, 66.65; H, 6.71; N, 15.54. Found: C, 66.29; H, 6.57; N, 15.90%. HPLC purity 98.1%.

### Example 6: SN39239 1-Cyclopentyl-6-((3-methoxyphenyl)amino)-3-methyl-1,3-dihydro-2H-imidazo[4,5-c]pyridin-2-one (12)

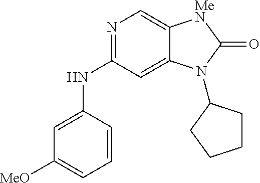 [SMILES: COc1cccc(Nc2cc3c(cn2)n(C)c(=O)n3C2CCCC2)c1]

A degassed mixture of chloride 6 (126 mg, 0.50 mmol), 3-methoxyaniline (74 mg, 0.60 mmol), Pd₂dba₃ (23 mg, 25 μmol), XPhos (47 mg, 102 μmol) and Cs₂CO₃ (326 mg, 1.02 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 12 (136 mg, 80%) as a tan powder: mp (EtOAc/pet ether) 66-69° C.; 1H NMR (CDCl₃) δ 7.83 (d, J=0.4 Hz, 1H, H-4), 7.22 (t, J=8.1 Hz, 1H, H-5′), 6.89 (t, J=2.3 Hz, 1H, H-2′), 6.82 (dd, J=8.0, 1.4 Hz, 1H, H-6′), 6.68 (d, J=0.6 Hz, 1H, H-7), 6.57 (ddd, J=8.2, 2.4, 0.7 Hz, 1H, H-4′), 6.49 (s, 1H, 6-NH), 4.79 (pent, J=8.8 Hz, 1H, 1-CH), 3.80 (s, 3H, 3′-OCH₃), 3.40 (s, 3H, 3-CH₃), 1.95-2.06 (m, 4H, 2×CH₂), 1.80-1.92 (m, 2H, CH₂), 1.62-1.72 (m, 2H, CH₂); MS m/z 339.2 (MH⁺, 100%). Anal calcd for C₁₉H₂₂N₄O·½H₂O: C, 65.69; H, 6.67; N, 16.13. Found: C, 65.42; H, 6.36; N, 15.92%. HPLC purity 99.7%.

### Example 7: SN39240 1-Cyclopentyl -6-(2-methoxyphenylamino)-3-methyl-1,3-dihydro-2H-imidazo[4,5-c]pyridin-2-one (13)

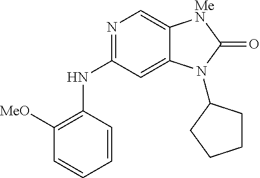 [SMILES: COc1ccccc1Nc1cc2c(cn1)n(C)c(=O)n2C1CCCC1]

A degassed mixture of chloride 6 (110 mg, 0.44 mmol), 2-methoxyaniline (65 mg, 0.52 mmol), Pd₂dba₃ (20 mg, 22 μmol), XPhos (42 mg, 88 μmol) and Cs₂CO₃ (285 mg, 0.88 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 13 (135 mg, 91%) as a tan powder: 1H NMR (CDCl₃) δ 7.83-7.90 (m, 2H, H-4, H-6′), 7.89-6.98 (m, 3H, H-3′, H-4′, H-5′), 6.83 (s, 1H, 6-NH), 6.61 (s, 1H, H-7), 4.78 (pent, J=8.8 Hz, 1H, 1-CH), 3.90 (s, 3H, 2′-OCH₃), 3.40 (s, 3H, 3-CH₃), 1.98-2.08 (m, 4H, 2×CH₂), 1.85-1.95 (m, 2H, CH₂), 1.63-1.75 (m, 2H, CH₂); MS m/z 338.4 (MH⁺, 100%); HRMS calcd for C₁₉H₂₃N₄O₂ (MH⁺) m/z 339.1816, found 339. 1819 (−1.0 ppm). HPLC purity 99.9%.

### Example 8: SN39241 6-((4-Chlorophenyl)amino)-1-cyclopentyl-3-methyl-1,3-dihydro-2H-imidazo[4,5-c]pyridin-2-one (14)

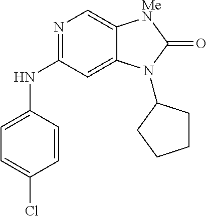 [SMILES: Cn1c(=O)n(C2CCCC2)c2cc(Nc3ccc(Cl)cc3)ncc21]

A degassed mixture of chloride 6 (119 mg, 0.47 mmol), 4-chloroaniline (72 mg, 0.57 mmol), Pd₂dba₃ (22 mg, 24 μmol), XPhos (45 mg, 95 μmol) and Cs₂CO₃ (308 mg, 0.95 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (40-60%) of EtOAc/pet. ether, to give imidazopyridinone 14 (80 mg, 49%) as a brown powder: mp (EtOAc/pet ether) 179-181° C.; ¹H NMR (CDCl₃) δ 7.83 (s, 1H, H-4), 7.22-7.18 (m, 4H, H-2′, H-3′, H-5′, H-6′), 6.54 (s, 1H, H-7), 6.43 (s, 1H, 6-NH), 4.76 (pent, J=8.8 Hz, 1H, 1-CH), 3.40 (s, 3H, 3-CH₃), 1.95-2.05 (m, 4H, 2×CH₂), 1.82-1.92 (m, 2H, CH₂), 1.63-1.75 (m, 2H, CH₂); MS m/z 343.2 (MH⁺, 100%), 345.2 (MH⁺, 35%). Anal calcd for C₁₈H₁₉ClN₄O·0.1EtOAc: C, 62.65; H, 5.76; N, 15.54. Found: C, 62.56; H, 5.62; N, 15.84%. HPLC purity 99.0%.

### Example 9: SN39242 6-((3-Chlorophenyl)amino)-1-cyclopentyl-3-methyl-1,3-dihydro-2H-imidazo[4,5-c]pyridin-2-one (15)

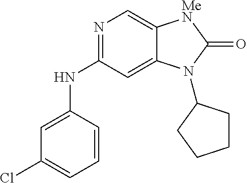 [SMILES: Cn1c(=O)n(C2CCCC2)c2cc(Nc3cccc(Cl)c3)ncc21]

A degassed mixture of chloride 6 (132 mg, 0.52 mmol), 3-chloroaniline (80 mg, 0.63 mmol), Pd₂dba₃ (24 mg, 26 μmol), XPhos (50 mg, 104 μmol) and Cs₂CO₃ (339 mg, 1.04 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-75%) of EtOAc/pet. ether, to give imidazopyridinone 15 (110 mg, 62%) as a tan powder: mp (EtOAc/pet ether) 145-147° C.; ¹H NMR (CDCl₃) δ 7.86 (d, J=0.6 Hz, 1H, H-4), 7.40 (t, J=2.1 Hz, 1H, H-2′), 7.22 (t, J=8.0 Hz, 1H, H-5′), 7.11 (ddd, J=8.2, 2.1, 0.9 Hz, 1H, H-6′), 6.95 (ddd, J=7.9, 2.0, 1.0 Hz, 1H, H-4′), 6.62 (d, J=0.6 Hz, 1H, H-7), 6.51 (s, 1H, 6-NH), 4.81 (pent, J=8.8 Hz, 1H, 1-CH), 3.41 (s, 3H, 3-CH₃), 1.97-2.08 (m, 4H, 2×CH₂), 1.84-1.94 (m, 2H, CH₂), 1.68-1.76 (m, 2H, CH₂); MS m/z 343.2 (MH⁺, 100%), 345.2 (MH⁺, 35%). Anal calcd for C₁₈H₁₉ClN₄O·0.1EtOAc: C, 62.85; H, 5.68; N, 15.93. Found: C, 62.73; H, 5.54; N, 15.94%. HPLC purity 99.9%.

### Example 10: SN39245 6-((2-Chlorophenyl)amino)-1-cyclopentyl-3-methyl-1,3-dihydro-2H-imidazo[4,5-c]pyridin-2-one (16)

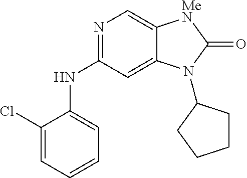 [SMILES: Cn1c(=O)n(C2CCCC2)c2cc(Nc3ccccc3Cl)ncc21]

A degassed mixture of chloride 6 (130 mg, 0.52 mmol), 2-chloroaniline (80 mg, 0.62 mmol), Pd₂dba₃ (24 mg, 26 μmol), XPhos (50 mg, 104 μmol) and Cs₂CO₃ (339 mg, 1.04 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (5-10%) of EtOAc/DCM, to give imidazopyridinone 16 (79 mg, 45%) as a brown foam: ¹H NMR (CDCl₃) δ 7.90 (dd, J=8.3, 1.5 Hz, 1H, H-6′), 7.87 (s, 1H, H-4), 7.39 (dd, J=8.0, 1.5 Hz, 1 H, H-3′), 7.23 (dt, J=8.5, 1.5 Hz, 1H, H-5′), 6.90 (dt, J=7.9, 1.5 Hz, 1H, H-4′), 6.74 (br s, 1H, 6-NH), 6.61 (s, 1H, H-7), 4.77 (pent, J=8.8 Hz, 1H, 1-CH), 3.41 (s, 3H, 3-CH₃), 1.98-2.08 (m, 4H, 2×CH₂), 1.86-1.96 (m, 2H, CH₂), 1.68-1.77 (m, 2H, CH₂); MS m/z 343.2 (MH⁺, 100%), 345.2 (MH⁺, 35%); HRMS calcd for C₁₈H₂₀ClN₄O (MH⁺) m/z 343.1320, found 343.1318 (0.5 ppm). HPLC purity 98.4%.

### Example 11: SN39246 1-Cyclopentyl-3-methyl-6-(4-nitrophenylamino)-1,3-dihydro-2H-imidazo[4,5-c]pyridin-2-one (17)

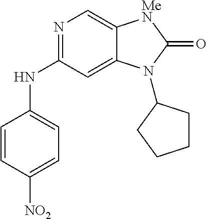 [SMILES: Cn1c(=O)n(C2CCCC2)c2cc(Nc3ccc([N+](=O)[O-])cc3)ncc21]

A degassed mixture of chloride 6 (131 mg, 0.52 mmol), 4-nitroaniline (86 mg, 0.63 mmol), Pd₂dba₃ (24 mg, 26 μmol), XPhos (50 mg, 104 μmol) and Cs₂CO₃ (339 mg, 1.04 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 17 (127 mg, 69%) as a red crystals: mp (EtOAc/pet ether) 270-272° C.; ¹H NMR (CDCl₃) δ 8.18 (ddd, J=9.2, 3.1, 2.1 Hz, 2 H, H-3′, H-5′), 7.94 (s, 1H, H-4), 7.46 (ddd, J=9.2, 3.1, 2.1 Hz, 2 H, H-2′, H-6′), 6.94 (br s, 1H, 6-NH), 6.64 (d, J=0.5 Hz, 1H, H-7), 4.82 (pent, J=8.8 Hz, 1H, 1-CH), 3.44 (s, 3H, 3-CH₃), 2.02-2.10 (m, 4H, 2×CH₂), 1.88-1.98 (m, 2H, CH₂), 1.70-1.80 (m, 2H, CH₂); MS m/z 354.2 (MH⁺, 100%). Anal calcd for C₁₈H₁₉N₅O₃: C, 61.18; H, 5.42; N, 19.82. Found: C, 61.29; H, 5.29; N, 19.94%. HPLC purity 100.0%.

### Example 12: SN39247 1-Cyclopentyl-3-methyl-6-(3-nitrophenylamino)-1,3-dihydro-2H-imidazo[4,5-c]pyridin-2-one (18)

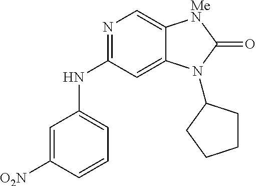 [SMILES: Cn1c(=O)n(C2CCCC2)c2cc(Nc3cccc([N+](=O)[O-])c3)ncc21]

A degassed mixture of chloride 6 (131 mg, 0.52 mmol), 3-nitroaniline (86 mg, 0.63 mmol), Pd₂dba₃ (24 mg, 26 μmol), XPhos (50 mg, 104 μmol) and Cs₂CO₃ (339 mg, 1.04 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-80%) of EtOAc/pet. ether, to give imidazopyridinone 18 (147 mg, 80%) as a yellow powder: mp (EtOAc/pet ether) 184-186° C.; ¹H NMR (CDCl₃) δ 8.36 (t, J=2.2 Hz, 1H, H-2′), 7.91 (s, 1H, H-4), 7.78 (ddd, J=8.1, 2.1, 0.8 Hz, 1H, H-6′), 7.59 (ddd, J=8.1, 2.0, 0.8 Hz, 1H, H-4′), 7.43 (t, J=8.1 Hz, 1H, H-5′), 6.75 (s, 1H, 6-NH), 6.61 (d, J=0.4 Hz, 1H, H-7), 4.83 (pent, J=8.8 Hz, 1H, 1-CH), 3.43 (s, 3H, 3-CH₃), 2.00-2.08 (m, 4H, 2×CH₂), 1.87-1.97 (m, 2H, CH₂), 1.70-1.78 (m, 2H, CH₂); MS m/z 354.2 (MH⁺, 100%). Anal. calcd for C₁₈H₁₉N₅O₃: C, 61.18; H, 5.42; N, 19.82. Found: C, 60.98; H, 5.29; N, 19.82%. HPLC purity 99.9%.

### Example 13: SN39263 1-Cyclopentyl-3-methyl-6-(2-nitrophenylamino)-1,3-dihydro-2H-imidazo[4,5-c]pyridin-2-one (19)

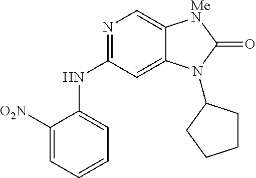 [SMILES: Cn1c(=O)n(C2CCCC2)c2cc(Nc3ccccc3[N+](=O)[O-])ncc21]

A degassed mixture of chloride 6 (131 mg, 0.52 mmol), 2-nitroaniline (86 mg, 0.63 mmol), Pd₂dba₃ (24 mg, 26 μmol), XPhos (50 mg, 104 μmol) and Cs₂CO₃ (339 mg, 1.04 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with 40% EtOAc/pet. ether, to give imidazopyridinone 19 (161 mg, 88%) as red crystals: ¹H NMR (CDCl₃) δ 10.10 (br s, 1H, 6-NH), 8.42 (dd, J=8.8, 1.2 Hz, 1H, H-3′), 8.32 (dd, J=8.6, 1.6 Hz, 1H, H-6′), 7.97 (s, 1H, H-4), 7.51 (ddd, J=8.4, 7.1, 1.4 Hz, 1H, H-5′), 6.90 (ddd, J=8.4, 7.1, 1.4 Hz, 1 H, H-4′), 6.68 (s, 1H, H-7), 4.81 (pent, J=8.8 Hz, 1H, 1-CH), 3.45 (s, 3H, 3-CH₃), 1.92-2.10 (m, 6H, 3×CH₂), 1.70-1.80 (m, 2H, CH₂); MS m/z 354.2 (MH⁺, 100%). Anal calcd for C₁₈H₁₉N₅O₃: C, 61.18; H, 5.42; N, 19.82. Found: C, 60.89; H, 5.34; N, 19.61%. HPLC purity 99.9%.

### Example 14: SN39273 N-(4-((1-Cyclopentyl-3-methyl-2-oxo-2,3-dihydro-1H-imidazo[4,5-c]pyridin-6-yl)amino)phenyl)acetamide (20)

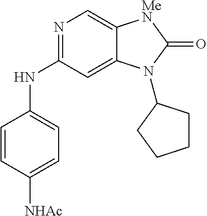 [SMILES: CC(=O)Nc1ccc(Nc2cc3c(cn2)n(C)c(=O)n3C2CCCC2)cc1]

A degassed mixture of chloride 6 (118 mg, 0.47 mmol), N-(4-aminophenyl)acetamide (85 mg, 0.56 mmol), Pd₂dba₃ (22 mg, 24 μmol), XPhos (45 mg, 94 μmol) and Cs₂CO₃ (306 mg, 0.94 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 20 (136 mg, 75%) as a cream powder: mp (EtOAc/pet ether) 152-155° C.; ¹H NMR (CDCl₃) δ 7.80 (s, 1H, H-4″), 7.45 (br d, J=8.8 Hz, 2 H, H-2′, H-6′), 7.20-7.30 (m, 3H, CONH, H-3′, H-5′), 6.55 (s, 1H, H-7), 6.43 (s, 1H, 4′-NH), 4.74 (pent, J=8.8 Hz, 1H, 1″-CH), 3.39 (s, 3H, 3″-CH₃), 2.17 (s, 3H, COCH₃), 1.95-2.04 (m, 4H, 2×CH₂), 1.82-1.92 (m, 2H, CH₂), 1.63-1.73 (m, 2H, CH₂); MS m/z 366.2 (MH⁺, 100%). Anal calcd for C₂₀H₂₃N₅O₂: C, 65.73; H, 6.34; N, 19.16. Found: C, 65.88; H, 6.44; N, 18.81%. HPLC purity 100.0%.

### Example 15: SN39280 N-(3-((1-Cyclopentyl-3-methyl-2-oxo-2,3-dihydro-1H-imidazo[4,5-c]pyridin-6-yl)amino)phenyl)acetamide (21)

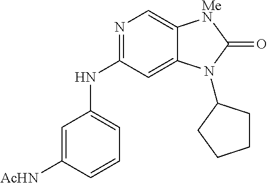 [SMILES: CC(=O)Nc1cccc(Nc2cc3c(cn2)n(C)c(=O)n3C2CCCC2)c1]

A degassed mixture of chloride 6 (110 mg, 0.44 mmol), N-(3-aminophenyl)acetamide (80 mg, 0.53 mmol), Pd₂dba₃ (20 mg, 22 μmol), XPhos (42 mg, 88 μmol) and Cs₂CO₃ (289 mg, 0.88 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (0-10% MeOH/EtOAc) gave imidazopyridinone 21 (130 mg, 81%) as tan crystals: mp (EtOAc) 215-217° C.; ¹H NMR (CDCl₃) δ 7.83 (s, 1H, H-4′), 7.78 (br s, 1H, H-2), 7.38 (br s, 1H, CONH), 7.22 (t, J=8.0 Hz, 1H, H-5), 7.04 (br dd, J=8.0, 1.3 Hz, 1H, H-6), 6.93 (d, J=7.9 Hz, 1H, H-4), 6.66 (s, 1H, H-7), 6.62 (s, 1H, 3-NH), 4.78 (pent, J=8.8 Hz, 1H, 1′-CH), 3.39 (s, 3H, 3′-CH₃), 2.16 (s, 3H, COCH₃), 1.98-2.07 (m, 4H, 2×CH₂), 1.84-1.92 (m, 2H, CH₂), 1.65-1.74 (m, 2H, CH₂); MS m/z 366.2 (MH⁺, 100%); HRMS calcd for C₂₀H₂₄N₅O₂ (MH⁺) m/z 366.1925, found 366.1920 (1.3 ppm). HPLC purity 99.3%.

### Example 16: SN39301 N-(2-((1-Cyclopentyl-3-methyl-2-oxo-2,3-dihydro-1H-imidazo[4,5-c]pyridin-6-yl)amino)phenyl)acetamide (22)

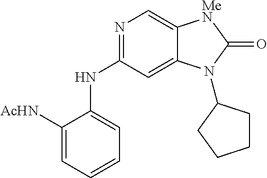 [SMILES: CC(=O)Nc1ccccc1Nc1cc2c(cn1)n(C)c(=O)n2C1CCCC1]

A degassed mixture of chloride 6 (133 mg, 0.53 mmol), N-(2-aminophenyl)acetamide (95 mg, 0.63 mmol), Pd₂dba₃ (24 mg, 27 μmol), XPhos (50 mg, 106 μmol) and Cs₂CO₃ (345 mg, 1.06 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-5%) of MeOH/EtOAc, to give imidazopyridinone 22 (185 mg, 96%) as a tan powder: mp (MeOH/EtOAc) 181-183° C.; ¹H NMR (CDCl₃) δ 8.11 (br s, 1H, CONH), 7.95 (d, J=7.4 Hz, 1H, H-6), 7.78 (s, 1H, H-4′), 7.30 (d, J=7.2 Hz, 1H, H-3), 7.18 (br dd, J=7.6, 6.6 Hz, 1H, H-5), 7.13 (br dd, J=7.7, 6.4 Hz, 1H, H-4), 6.39 (br s, 1H, 6′-NH), 6.18 (s, 1H, H-7), 4.68 (pent, J=8.7 Hz, 1H, 1-CH), 3.38 (s, 3H, 3-CH₃), 2.12 (s, 3H, COCH₃), 1.89-2.00 (m, 4H, 2×CH₂), 1.75-1.85 (m, 2H, CH₂), 1.60-1.68 (m, 2H, CH₂); MS m/z 366.2 (MH⁺, 100%); HRMS calcd for C₂₀H₂₄N₅O₂ (MH⁺) m/z 365.1852, found 365.1872 (−5.4 ppm).

HPLC purity 98.9%.

### Example 17: SN39275 4-((1-Cyclopentyl-3-methyl-2-oxo-2,3-dihydro-1H-imidazo[4,5-c]pyridin-6-yl)amino)benzonitrile (23)

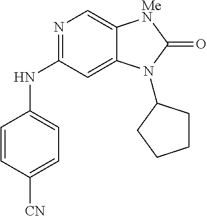 [SMILES: Cn1c(=O)n(C2CCCC2)c2cc(Nc3ccc(C#N)cc3)ncc21]

A degassed mixture of chloride 6 (122 mg, 0.49 mmol), 4-aminobenzonitrile (69 mg, 0.58 mmol), Pd₂dba₃ (22 mg, 25 μmol), XPhos (47 mg, 98 μmol) and Cs₂CO₃ (319 mg, 0.98 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with wate39278 mL), washed with brine (30 mL), dried (MgSO₄), filtered and the solvent evaporated. The residue was purified by chromatography (60-80% EtOAc/pet. ether) to give 23 (131 mg, 81%) as a cream powder: mp (EtOAc/pet ether) 236-238° C.; ¹H NMR (CDCl₃) δ 7.91 (s, 1H, H-4′), 7.45 (ddd, J=8.8, 2.3, 2.0 Hz, 2 H, H-3, H-5), 7.43 (ddd, J=8.8, 2.3, 2.0 Hz, 2 H, H-2, H-6), 6.77 (s, 1H, 4-NH), 6.60 (s, 1H, H-7), 4.81 (pent, J=8.8 Hz, 1H, 1′-CH), 3.43 (s, 3H, 3′-CH₃), 1.88-2.09 (m, 6H, 3×CH₂), 1.68-1.78 (m, 2H, CH₂); MS m/z 351.2 (MH⁺, 100%). Anal calcd for C₁₉H₁₉N₅O: C, 68.45; H, 5.74; N, 21.01. Found: C, 68.24; H, 5.84; N, 20.86%. HPLC purity 100.0%.

### Example 18: SN39291 3-((1-Cyclopentyl-3-methyl-2-oxo-2,3-dihydro-1H-imidazo[4,5-c]pyridin-6-yl)amino)benzonitrile (24)

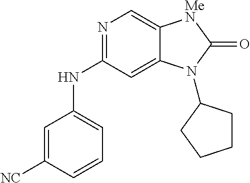 [SMILES: Cn1c(=O)n(C2CCCC2)c2cc(Nc3cccc(C#N)c3)ncc21]

A degassed mixture of chloride 6 (122 mg, 0.49 mmol), 3-aminobenzonitrile (69 mg, 0.58 mmol), Pd₂dba₃ (22 mg, 25 μmol), XPhos (47 mg, 98 μmol) and Cs₂CO₃ (319 mg, 0.98 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-60%) of EtOAc/pet. ether, to give imidazopyridinone 24 (120 mg, 74%) as a cream powder: mp (EtOAc/pet ether) 233-235° C.; ¹H NMR (CDCl₃) δ 7.88 (s, 1H, H-4), 7.85 (t, J=1.8 Hz, 1H, H-2′), 7.50 (ddd, J=8.3, 2.3, 1.0 Hz, 1H, H-6′), 7.36 (t, J=8.0 Hz, 1H, H-5′), 7.22 (dt, J=7.6, 1.2 Hz, 1H, H-4′), 6.61 (br s, 1H, 6-NH), 6.53 (s, 1H, H-7), 4.81 (pent, J=8.8 Hz, 1H, 1-CH), 3.42 (s, 3H, 3-CH₃), 1.98-2.08 (m, 4H, 2×CH₂), 1.87-1.97 (m, 2H, CH₂), 1.69-1.79 (m, 2H, CH₂); MS m/z 334.2 (MH⁺, 100%). Anal calcd for C₁₉H₁₉N₅O·0.1EtOAc: C, 68.09; H, 5.83; N, 20.47. Found: C, 67.93; H, 6.05; N, 20.47%. HPLC purity 100.0%.

### Example 19: SN39297 2-((1-Cyclopentyl-3-methyl-2-oxo-2,3-dihydro-1H-imidazo[4,5-c]pyridin-6-yl)amino)benzonitrile (25)

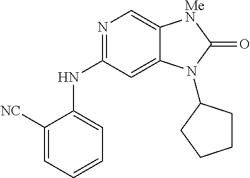 [SMILES: Cn1c(=O)n(C2CCCC2)c2cc(Nc3ccccc3C#N)ncc21]

A degassed mixture of chloride 6 (120 mg, 0.48 mmol), 2-aminobenzonitrile (68 mg, 0.57 mmol), Pd₂dba₃ (22 mg, 24 μmol), XPhos (46 mg, 96 μmol) and Cs₂CO₃ (313 mg, 0.96 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 25 (101 mg, 64%) as orange crystals: mp (MeOH/EtOAc) 171-173° C.; ¹H NMR (CDCl₃) δ 8.83 (s, 1H, H-4), 8.13 (br s, 1H, 6-NH), 7.90 (dd, J=8.2, 1.0 Hz, 1H, H-6′), 7.63 (ddd, J=8.3, 6.9, 1.3 Hz, 1H, H-4′), 7.55 (dd, J=8.3, 1.0 Hz, 1H, H-3′), 7.30 (ddd, J=8.2, 6.9, 1.2 Hz, 1H, H-5′), 6.76 (s, 1H, H-7), 4.82 (pent, J=8.8 Hz, 1 H, 1-CH), 3.49 (s, 3H, 3-CH₃), 1.95-2.15 (m, 6H, 3×CH₂), 1.68-1.80 (m, 2H, CH₂); MS m/z 334.2 (MH⁺, 100%). Anal. calcd for C₁₉H₁₉N₅O·1.3CH₃₀H: C, 65.01; H, 6.50; N, 18.67. Found C, 64.71; H, 6.41; N, 18.99%. HPLC purity 98.8%.

### Example 20: SN39278 1-Cyclopentyl-3-methyl-6-((4-(trifluoromethyl)phenyl)amino)-1,3-dihydro-2H-imidazo[4,5-c]pyridin-2-one (26)

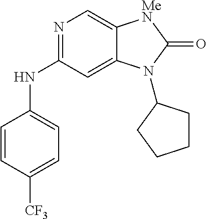 [SMILES: Cn1c(=O)n(C2CCCC2)c2cc(Nc3ccc(C(F)(F)F)cc3)ncc21]

A degassed mixture of chloride 6 (111 mg, 0.44 mmol), 4-(trifluoromethyl)aniline (85 mg, 0.53 mmol), Pd₂dba₃ (20 mg, 22 μmol), XPhos (42 mg, 88 μmol) and Cs₂CO₃ (289 mg, 0.88 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-70% EtOAc/pet. ether) to give 26 (125 mg, 75%) as a cream powder: mp (EtOAc/pet ether) 196-199° C.; ¹H NMR (CDCl₃) δ 7.88 (d, J=0.4 Hz, 1H, H-4), 7.53 (d, J=8.6 Hz, 2 H, H-3′, H-5′), 7.39 (d, J=8.5 Hz, 2 H, H-2′, H-6′), 6.66 (br s, 1H, 6-NH), 6.63 (s, 1H, H-7), 4.80 (pent, J=8.8 Hz, 1H, 1-CH), 3.42 (s, 3H, 3-CH₃), 1.99-2.07 (m, 4H, 2×CH₂), 1.87-1.95 (m, 2H, CH₂), 1.68-1.78 (m, 2H, CH₂); ¹³C NMR (CDCl₃) δ 154.2, 149.4, 145.0, 137.0, 126.8 (q, J=3.8 Hz), 125.9 (2), 124.7 (q, J=233.6 Hz), 123.7, 123.0 (q, J=32.8 Hz), 117.1 (2), 91.2, 53.9, 29.1 (2), 27.6, 25.3 (2); MS m/z 377.2 (MH⁺, 100%). Anal. calcd for C₁₉H₁₉F₃N₄O: C, 60.63; H, 5.09; N, 14.89. Found: C, 60.68; H, 5.30; N, 15.02%. HPLC purity 99.7%.

### Example 21: SN39290 1-Cyclopentyl-3-methyl-6-((3-(trifluoromethyl)phenyl)amino)-1,3-dihydro-2H-imidazo[4,5-c]pyridin-2-one (27)

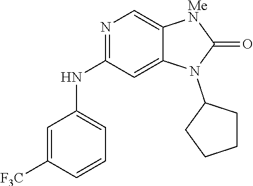 [SMILES: Cn1c(=O)n(C2CCCC2)c2cc(Nc3cccc(C(F)(F)F)c3)ncc21]

A degassed mixture of chloride 6 (125 mg, 0.50 mmol), 3-(trifluoromethyl)aniline (96 mg, 0.60 mmol), Pd₂dba₃ (23 mg, 25 μmol), XPhos (48 mg, 100 μmol) and Cs₂CO₃ (326 mg, 1.00 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (40-50% EtOAc/pet. ether) gave imidazopyridinone 27 (146 mg, 78%) as cream needles: mp (EtOAc) 162-163° C.; ¹H NMR (CDCl₃) δ 7.87 (s, 1H, H-4), 7.78 (s, 1H, H-2′), 7.40-7.43 (m, 2H, H-5′, H-6′), 7.21-7.23 (m, 1H, H-4′), 6.68 (br s, 1H, 6-NH), 6.63 (d, J=0.4 Hz, 1H, H-7), 4.83 (pent, J=8.8 Hz, 1H, 1-CH), 3.42 (s, 3H, 3-CH₃), 1.96-2.07 (m, 4H, 2×CH₂), 1.83-1.92 (m, 2H, CH₂), 1.68-1.76 (m, 2H, CH₂); ¹³C NMR (CDCl₃) δ 154.3, 149.8, 142.4, 136.9, 131.8 (q, J=32.1 Hz), 130.0, 126.1, 124.3 (q, J=272.3), 123.5, 121.6, 118.2 (q, J=3.8 Hz), 114.5 (q, J=3.9 Hz), 90.4, 53.7, 29.2 (2), 27.6, 25.3 (2); MS m/z 377.2 (MH⁺, 100%); HRMS calcd for C₁₉H₂₀F₃N₄O (MH⁺) m/z 377.1584, found 377.1585 (−0.4 ppm). HPLC purity 99.3%.

### Example 22: SN39283 1-Cyclopentyl-3-methyl-6-((2-(trifluoromethyl)phenyl)amino)-1,3-dihydro-2H-imidazo[4,5-c]pyridin-2-one (28)

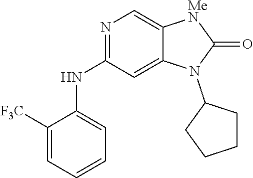 [SMILES: Cn1c(=O)n(C2CCCC2)c2cc(Nc3ccccc3C(F)(F)F)ncc21]

A degassed mixture of chloride 6 (114 mg, 0.45 mmol), 2-trifluoroaniline (88 mg, 0.54 mmol), Pd₂dba₃ (21 mg, 23 μmol), XPhos (43 mg, 90 μmol) and Cs₂CO₃ (293 mg, 0.90 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (10-50%) of EtOAc/pet. ether, to give imidazopyridinone 28 (110 mg, 65%) as a grey foam: 1H NMR (CDCl₃) δ 7.87 (s, 1H, H-4), 7.76 (d, J=8.3 Hz, 1H, H-3′), 7.62 (dd, J=7.8, 0.7 Hz, 1 H, H-6′), 7.47 (br dd, J=8.0, 7.6 Hz, 1H, H-4′), 7.07 (br dd, J=7.7, 765 Hz, 1H, H-5′), 6.63 (br s, 1H, 6-NH), 6.58 (d, J=0.5 Hz, 1H, H-7), 4.76 (pent, J=8.8 Hz, 1H, 1-CH), 3.41 (s, 3H, 3-CH₃), 1.97-2.06 (m, 4H, 2×CH₂), 1.83-1.93 (m, 2H, CH₂), 1.62-1.74 (m, 2H, CH₂); MS m/z 377.2 (MH⁺, 100%); HRMS calcd for C₁₉H₁₉F₃N₄O (MH⁺) m/z 376.1511, found 376.1535 (6.3 ppm). HPLC purity 99.7%.

### Example 23: SN39274 6-((4-Acetylphenyl)amino)-1-cyclopentyl-3-methyl-1,3-dihydro-2H-imidazo[4,5-c]pyridin-2-one (29)

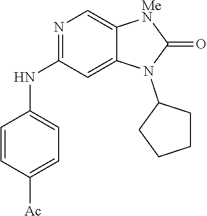 [SMILES: CC(=O)c1ccc(Nc2cc3c(cn2)n(C)c(=O)n3C2CCCC2)cc1]

A degassed mixture of chloride 6 (117 mg, 0.47 mmol), 1-(4-aminophenyl)ethan-1-one (75 mg, 0.56 mmol), Pd₂dba₃ (22 mg, 24 μmol), XPhos (45 mg, 94 μmol) and Cs₂CO₃ (326 mg, 0.94 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (60-100% EtOAc/pet. ether) to give imidazopyridinone 29 (131 mg, 80%) as a lemon powder: mp (EtOAc/pet ether) 181-183° C.; ¹H NMR (CDCl₃) δ 7.89-7.95 (m, 3H, H-4, H-3′, H-5′), 7.36 (ddd, J=8.8, 2.6, 1.9 Hz, 2 H, H-2′, H-6′), 6.82 (s, 1H, 6-NH), 6.69 (d, J=0.5 Hz, 1H, H-7″), 4.81 (pent, J=8.8 Hz, 1H, 1-CH), 3.43 (s, 3H, 3-CH₃), 2.56 (s, 3H, COCH₃), 2.00-2.08 (m, 4H, 2×CH₂), 1.87-1.97 (m, 2H, CH₂), 1.70-1.78 (m, 2H, CH₂); MS m/z 351.2 (MH⁺, 100%). Anal calcd for C₂₀H₂₂N₄O₂·0.1EtOAc: C, 68.21; H, 6.40; N, 15.60. Found: C, 68.09; H, 6.52; N, 15.37%. HPLC purity 97.9%.

### Example 24: SN39279 6-((3-Acetylphenyl)amino)-1-cyclopentyl-3-methyl-1,3-dihydro-2H-imidazo[4,5-c]pyridin-2-one (30)

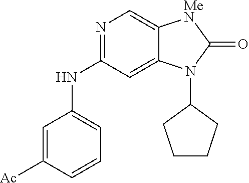 [SMILES: CC(=O)c1cccc(Nc2cc3c(cn2)n(C)c(=O)n3C2CCCC2)c1]

A degassed mixture of chloride 6 (110 mg, 0.44 mmol), 1-(3-aminophenyl)ethan-1-one (71 mg, 0.53 mmol), Pd₂dba₃ (22 mg, 20 μmol), XPhos (42 mg, 88 μmol) and Cs₂CO₃ (287 mg, 0.88 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-100% EtOAc/pet. ether) to give imidazopyridinone 30 (147 mg, 80%) as a cream powder: mp (EtOAc/pet ether) 125-127° C.; ¹H NMR (CDCl₃) δ 7.93 (t, J=1.9 Hz, 1H, H-2′), 7.86 (s, 1H, H-4), 7.56 (ddd, J=7.6, 1.4, 1.1 Hz, 1H, H-6′), 7.53 (ddd, J=8.1, 2.3, 0.9 Hz, 1H, H-4′), 7.40 (t, J=7.8 Hz, 1H, H-5′), 6.63 (s, 1H, H-7), 6.61 (br s, 1H, 6-NH), 4.81 (pent, J=8.8 Hz, 1H, 1-CH), 3.41 (s, 3H, 3-CH₃), 2.60 (s, 3H, COCH₃), 1.98-2.07 (m, 4H, 2×CH₂), 1.83-1.93 (m, 2H, CH₂), 1.67-1.76 (m, 2H, CH₂); MS m/z 351.2 (MH⁺, 100%). Anal. calcd for C₂₀H₂₂N₄₂: C, 68.55; H, 6.33; N, 15.99. Found: C, 68.54; H, 6.52; N, 16.03%. HPLC purity 99.6%.

### Example 25: SN39304 1-Cyclopentyl-3-methyl-6-((4-(methylsulfonyl)phenyl)amino)-1,3-dihydro-2H-imidazo[4,5-c]pyridin-2-one (31)

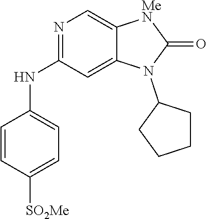 [SMILES: Cn1c(=O)n(C2CCCC2)c2cc(Nc3ccc(S(C)(=O)=O)cc3)ncc21]

A degassed mixture of chloride 6 (125 mg, 0.50 mmol), 4-(methylsulfonyl)aniline (102 mg, 0.60 mmol), Pd₂dba₃ (23 mg, 25 μmol), XPhos (48 mg, 100 μmol) and Cs₂CO₃ (326 mg, 1.00 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 31 (154 mg, 80%) as cream needles: mp (EtOAc/pet ether) 161-164° C.; ¹H NMR (CDCl₃) δ 7.92 (s, 1H, H-4), 7.82 (ddd, J=8.8, 2.6, 1.9 Hz, 2 H, H-2′, H-6′), 7.51 (ddd, J=8.8, 2.6, 1.9 Hz, 2 H, H-3′, H-5′), 6.86 (s, 1H, 6-NH), 6.63 (d, J=0.3 Hz, 1H, H-7), 4.81 (pent, J=8.8 Hz, 1H, 1-CH), 3.43 (s, 3H, 3-CH₃), 3.04 (s, 3H, SO₂CH₃), 2.00-2.08 (m, 4H, 2×CH₂), 1.88-1.98 (m, 2H, CH₂), 1.88-1.98 (m, 2H, CH₂); MS m/z 387.2 (MH⁺, 100%). Anal. calcd for C₁₉H₂₂N₄O₃S/2EtOAc: C, 58.59; H, 6.09; N, 13.01. Found: C, 58.86; H, 6.30; N, 13.20%. HPLC purity 99.9%.

### Example 26: SN39308 1-Cyclopentyl-3-methyl-6-((3-(methylsulfonyl)phenyl)amino)-1,3-dihydro-2H-imidazo[4,5-c]pyridin-2-one (32)

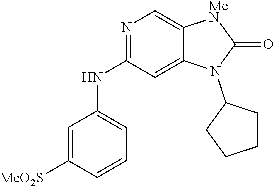 [SMILES: Cn1c(=O)n(C2CCCC2)c2cc(Nc3cccc(S(C)(=O)=O)c3)ncc21]

A degassed mixture of chloride 6 (112 mg, 0.45 mmol), 3-(methylsulfonyl)aniline. HCl (111 mg, 0.53 mmol), Pd₂dba₃ (21 mg, 23 μmol), XPhos (43 mg, 90 μmol) and Cs₂CO₃ (484 mg, 1.49 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (80-100%) of EtOAc/pet. ether, to give imidazopyridinone 32 (154 mg, 90%) as a white powder: mp (EtOAc/pet ether) 208-211° C.; ¹H NMR (CDCl₃) δ 8.00 (d, J=2.8 Hz, 1H, H-2′), 7.89 (s, 1H, H-4), 7.61-7.66 (m, 1H, H-5′), 7.44-7.49 (m, 2H, H-4′, H-6′), 6.84 (s, 1H, 6-NH), 6.60 (d, J=0.5 Hz, 1H, H-7), 4.82 (pent, J=8.8 Hz, 1H, 1-CH), 3.42 (s, 3H, 3-CH₃), 3.07 (s, 3H, SO₂CH₃), 2.00-2.08 (m, 4H, 2×CH₂), 1.87-1.97 (m, 2H, CH₂), 1.68-1.77 (m, 2H, CH₂); MS m/z 387.2 (MH⁺, 100%). Anal. calcd for C₁₉H₂₂N₄O₃S·½EtOAc: C, 58.59; H, 6.09; N, 13.01. Found: C, 58.73; H, 6.25; N, 13.28%. HPLC purity 99.8%.

### Example 27: SN39330 1-Cyclopentyl-3-methyl-6-((2-(methylsulfonyl)phenyl)amino)-1,3-dihydro-2H-imidazo[4,5-c]pyridin-2-one (33)

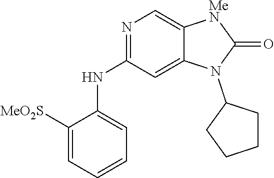 [SMILES: Cn1c(=O)n(C2CCCC2)c2cc(Nc3ccccc3S(C)(=O)=O)ncc21]

A degassed mixture of chloride 6 (126 mg, 0.50 mmol), 2-(methylsulfonyl)aniline (103 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-80%) of EtOAc/pet. ether, to give imidazopyridinone 33 (168 mg, 87%) as a white powder: mp (EtOAc/pet. ether) 178-181° C.; ¹H NMR (CDCl₃) δ 8.42 (br s, 1H, 6-NH), 8.11 (dd, J=8.4, 0.7 Hz, 1H, H-3′), 7.87-7.92 (m, 2H, H-4, H-6′), 7.53 (ddd, J=8.6, 7.1, 1.6 Hz, 1H, H-5′), 7.04 (ddd, J=8.2, 7.1, 1.0 Hz, 1H, H-4′), 6.55 (d, J=0.5 Hz, 1H, H-7), 4.78 (pent, J=8.8 Hz, 1H, 1-CH), 3.43 (s, 3H, 3-CH₃), 3.12 (s, 3H, 2′-SO₂CH₃), 1.93-2.10 (m, 6H, 3×CH₂), 1.70-1.80 (m, 2H, CH₂); MS m/z 387.2 (MH⁺, 100%); HRMS calcd for C₁₉H₂₃N₄O₃ (MH⁺) m/z 387.1485, found 387.1493 (−1.9 ppm). HPLC purity 100.0%.

### Example 28: SN39302 6-((4-(Benzyloxy)phenyl)amino)-1-cyclopentyl-3-methyl-1,3-dihydro-2H-imidazo[4,5-c]pyridin-2-one (34)

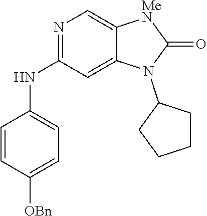 [SMILES: Cn1c(=O)n(C2CCCC2)c2cc(Nc3ccc(OCc4ccccc4)cc3)ncc21]

A degassed mixture of chloride 6 (200 mg, 0.80 mmol), 4-(benzyloxy)aniline. HCl (225 mg, 0.95 mmol), Pd₂dba₃ (37 mg, 40 μmol), XPhos (76 mg, 160 μmol) and Cs₂CO₃ (860 mg, 2.64 mmol) in dioxane (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-80%) of EtOAc/pet. ether, to give imidazopyridinone 34 (215 mg, 65%) as tan crystals: mp (EtOAc/pet ether) 144-147° C.; ¹H NMR (CDCl₃) δ 7.77 (s, 1H, H-4), 7.45 (br d, J=7.2 Hz, 2 H, H-2″, H-6″), 7.39 (br dd, J=7.6, 7.1 Hz, 2 H, H-3″, H-5″), 7.33 (br t, J=7.1 Hz, 1H, H-4″), 7.20 (ddd, J=8.9, 3.5, 2.2 Hz, 2 H, H-2′, H-6′), 6.97 (ddd, J=8.9, 3.5, 2.2 Hz, 2 H, H-3′, H-5′), 6.42 (d, J=0.5 Hz, 1H, H-7), 6.29 (s, 1H, 6-NH), 5.07 (s, 2H, CH₂O), 4.73 (pent, J=8.8 Hz, 1H, 1-CH), 3.38 (s, 3H, 3-CH₃), 1.93-2.01 (m, 4H, 2×CH₂), 1.76-1.86 (m, 2H, CH₂), 1.60-1.70 (m, 2H, CH₂); MS m/z 415.2 (MH⁺, 100%). Anal calcd for C₂₅H₂₆N₄O₂·¼EtOAc: C, 71.54; H, 6.47; N, 12.83. Found: C, 71.26; H, 6.44; N, 12.72%. HPLC purity 99.9%.

### Example 29: SN39303 6-((3(Benzyloxy)phenyl)amino)-1-cyclopentyl-3-methyl-1,3-dihydro-2H-imidazo[4,5-c]pyridin-2-one (35)

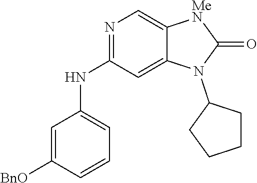 [SMILES: Cn1c(=O)n(C2CCCC2)c2cc(Nc3cccc(OCc4ccccc4)c3)ncc21]

A degassed mixture of chloride 6 (203 mg, 0.81 mmol), 3-(benzyloxy)aniline (193 mg, 0.97 mmol), Pd₂dba₃ (37 mg, 40 μmol), XPhos (77 mg, 162 μmol) and Cs₂CO₃ (528 mg, 1.62 mmol) in dioxane (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-80%) of EtOAc/pet. ether, to give imidazopyridinone 35 (246 mg, 74%) as white needles: mp (EtOAc/pet ether) 110-112° C.; ¹H NMR (CDCl₃) δ 7.85 (d, J=0.4 Hz, 1H, H-4), 7.38-7.46 (m, 4H, H-2″, H-3″, H-5″, H-6″), 7.36 (br tt, J=7.0, 1.5 Hz, 1H, H-4″), 7.24 (t, J=8.1 Hz, 1H, H-5′), 7.01 (t, J=2.2 Hz, 1H, H-2′), 6.86 (dd, J=8.0, 1.0 Hz, 1H, H-6), 6.71 (d, J=0.6 Hz, 1H, H-7), 6.65 (ddd, J=8.2, 2.4, 0.7 Hz, 1H, H-4′), 6.51 (br s, 1H, 6-NH), 5.08 (s, 2H, CH₂O), 4.81 (pent, J=8.8 Hz, 1H, 1-CH), 3.42 (s, 3H, 3-CH₃), 1.99-2.08 (m, 4H, 2×CH₂), 1.85-1.96 (m, 2H, CH₂), 1.67-1.77 (m, 2H, CH₂); MS m/z 415.2 (MH⁺, 100%). Anal calcd for C₂₅H₂₆N₄O₂: C, 72.44; H, 6.32; N, 13.52. Found: C, 71.16; H, 6.36; N, 13.19%. HPLC purity 100.0%.

### Example 30: SN39328 6-((2-(Benzyloxy)phenyl)amino)-1-cyclopentyl-3-methyl-1,3-dihydro-2H-imidazo[4,5-c]pyridin-2-one (36)

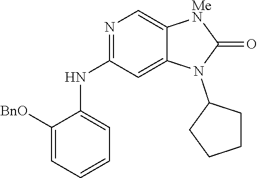 [SMILES: Cn1c(=O)n(C2CCCC2)c2cc(Nc3ccccc3OCc3ccccc3)ncc21]

A degassed mixture of chloride 6 (164 mg, 0.65 mmol), 2-(benzyloxy)aniline (156 mg, 0.78 mmol), Pd₂dba₃ (30 mg, 33 μmol), XPhos (63 mg, 130 μmol) and Cs₂CO₃ (466 mg, 1.43 mmol) in dioxane (10 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (40-50%) of EtOAc/pet. ether, to give imidazopyridinone 36 (166 mg, 61%) as a white powder: mp (EtOAc/pet. ether) 170-173° C.; ¹H NMR (CDCl₃) δ 7.84-7.88 (m, 2H, H-4, H-6′), 7.42-7.46 (m, 2H, H-2″, H-6″), 7.32-7.40 (m, 3H, H-3″, H-4″, H-5″), 6.94-6.99 (m, 2H, H-3′, H-4′), 6.87-6.89 (m, 1H, H-5′), 6.85 (br s, 1H, 6-NH), 6.61 (d, J=0.5 Hz, 1H, H-7), 5.15 (s, 2H, CH₂O), 4.77 (pent, J=8.8 Hz, 1H, 1-CH), 3.39 (s, 3H, 3-CH₃), 1.97-2.05 (m, 4H, 2×CH₂), 1.84-1.93 (m, 2H, CH₂), 1.67-1.75 (m, 2H, CH₂); MS m/z 415.2 (MH⁺, 100%); HRMS calcd for C₂₅H₂₇N₄O₂ (MH⁺) m/z 415.2129, found 415.2135 (−1.7 ppm). HPLC purity 99.6%.

### Example 31: SN39309 2-(4-((1-Cyclopentyl-3-methyl-2-oxo-2,3-dihydro-1H-imidazo[4,5-c]pyridin-6-yl)amino)phenyl)-2-methylpropanenitrile (37)

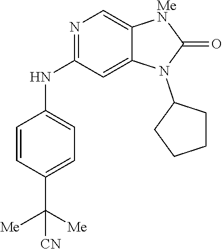 [SMILES: Cn1c(=O)n(C2CCCC2)c2cc(Nc3ccc(C(C)(C)C#N)cc3)ncc21]

A degassed mixture of chloride 6 (126 mg, 0.50 mmol), 2-(4-aminophenyl)-2-methylpropanenitrile (96 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 37 (160 mg, 85%) as a tan powder: mp (MeOH/EtOAc) 144-146° C.; 1H NMR (CDCl₃) δ 7.84 (d, J=0.5 Hz, 1H, H-4″), 7.41 (ddd, J=8.8, 2.7, 2.1 Hz, 2 H, H-3′, H-5′), 7.32 (ddd, J=8.8, 2.7, 2.1 Hz, 2 H, H-2′, H-6′), 6.58 (d, J=0.6 Hz, 1H, H-7), 6.50 (s, 1H, 6″-NH), 4.78 (pent, J=8.8 Hz, 1H, 1-CH), 3.40 (s, 3H, 3″-CH₃), 1.99-2.07 (m, 4H, 2×CH₂), 1.86-1.95 (m, 2H, CH₂), 1.68-1.77 (m, 8H, 2-CH₃, H-3, CH₂); MS m/z 376.2 (MH⁺, 100%). Anal. calcd for C₁₉H₂₅N₅O·¼CH₃OH: C, 69.69; H, 6.83; N, 18.26. Found: C, 69.37; H, 6.37; N, 18.20%. HPLC purity 99.4%.

### Example 32: SN39385 1-Cyclopentyl-6-((2,4-dimethylphenyl)amino)-3-methyl-1,3-dihydro-2H-imidazo[4,5-c]pyridin-2-one (38)

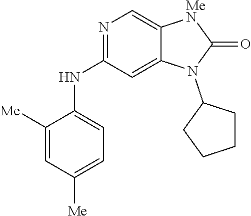 [SMILES: Cc1ccc(Nc2cc3c(cn2)n(C)c(=O)n3C2CCCC2)c(C)c1]

A degassed mixture of chloride 6 (128 mg, 0.51 mmol), 2,4-dimethylaniline (74 mg, 0.61 mmol), Pd₂dba₃ (23 mg, 25 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 38 (58 mg, 34%) as a white powder: mp (EtOAc/pet ether) 173-175° C.; ¹H NMR (CDCl₃) δ 7.78 (d, J=0.6 Hz, 1H, H-4), 7.24 (d, J=8.0 Hz, 1H, H-6′), 7.08 (s, 1H, H-3′), 7.01 (d, J=8.0 Hz, 1H, H-5′), 6.34 (d, J=0.6 Hz, 1H, H-7), 6.06 (br s, 1H, 6-NH), 4.70 (pent, J=8.8 Hz, 1H, 1-CH), 3.38 (s, 3H, 3-CH₃), 2.34 (s, 3H, 4′-CH₃), 2.24 (s, 3H, 2′-CH₃), 1.92-1.98 (m, 4H, 2×CH₂), 1.75-1.85 (m, 2H, CH₂), 1.58-1.68 (m, 2H, CH₂); MS m/z 337.2 (MH⁺, 100%); HRMS calcd for C₂₀H₂₅N₄O (MH⁺) m/z 337.2010, found 337.2008 (0.4 ppm). HPLC purity 99.6%.

### Example 33: SN39390 1-Cyclopentyl-6-((2,3-dimethylphenyl)amino)-3-methyl-1,3-dihydro-2H-imidazo[4,5-c]pyridin-2-one (39)

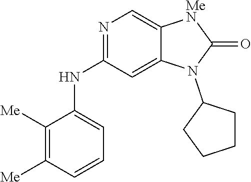 [SMILES: Cc1cccc(Nc2cc3c(cn2)n(C)c(=O)n3C2CCCC2)c1C]

A degassed mixture of chloride 6 (126 mg, 0.50 mmol), 2,3-dimethylaniline (73 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 39 (47 mg, 28%) as a tan foam: ¹H NMR (CDCl₃) δ 7.79 (s, 1H, H-4), 7.20 (d, J=7.9 Hz, 1H, H-6′), 7.10 (dd, J=7.8, 7.6 Hz, 1H, H-5′), 6.98 (d, J=7.4 Hz, 1H, H-4′), 6.31 (d, J=0.4 Hz, 1H, H-7), 6.20 (br s, 1 H, 6-NH), 4.71 (pent, J=8.8 Hz, 1H, 1-CH), 3.38 (s, 3H, 3-CH₃), 2.34 (s, 3H, 3′-CH₃), 2.19 (s, 3H, 2′-CH₃), 1.89-1.98 (m, 4H, 2×CH₂), 1.70-1.80 (m, 2H, CH₂), 1.58-1.67 (m, 2H, CH₂); MS m/z 337.2 (MH⁺, 100%). HRMS calcd for C₂₀H₂₅N₄O (MH⁺) m/z 337.2023, found 337.2017 (1.7 ppm). HPLC purity 99.1%.

### Example 34: SN39393 1-Cyclopentyl-6-((3,4-dimethylphenyl)amino)-3-methyl-1,3-dihydro-2H-imidazo[4,5-c]pyridin-2-one (40)

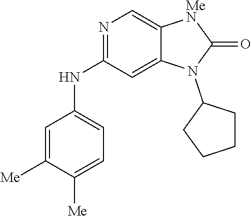 [SMILES: Cc1ccc(Nc2cc3c(cn2)n(C)c(=O)n3C2CCCC2)cc1C]

A degassed mixture of chloride 6 (129 mg, 0.51 mmol), 3,4-dimethylaniline (75 mg, 0.61 mmol), Pd₂dba₃ (23 mg, 25 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 40 (99 mg, 57%) as brown cubes: mp (EtOAc/pet ether) 150-151° C.; ¹H NMR (CDCl₃) δ 7.80 (d, J=0.5 Hz, 1H, H-4), 7.08 (d, J=8.0 Hz, 1H, H-5′), 7.04 (d, J=2.3 Hz, 1H, H-2′), 6.99 (dd, J=8.0, 2.3 Hz, 1 H, H-6′), 6.64 (d, J=0.7 Hz, 1H, H-7), 6.40 (br s, 1H, 6-NH), 4.78 (pent, J=8.8 Hz, 1H, 1-CH), 3.39 (s, 3H, 3-CH₃), 2.25 (s, 3H, 3′-CH₃), 2.24 (s, 3H, 2′-CH₃), 1.96-2.04 (m, 4H, 2×CH₂), 1.80-1.90 (m, 2H, CH₂), 1.64-1.74 (m, 2H, CH₂); MS m/z 337.2 (MH⁺, 100%). HRMS calcd for C₂₀H25N₄O (MH⁺) m/z 337.2023, found 337.2017 (1.7 ppm). HPLC purity 99.9%.

### Example 35: SN39378 6-((3-Chloro-2-methylphenyl)amino)-1-cyclopentyl-3-methyl-1,3-dihydro-2H-imidazo[4,5-c]pyridin-2-one (41)

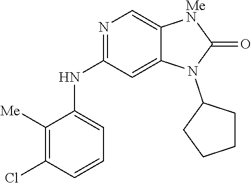 [SMILES: Cc1c(Cl)cccc1Nc1cc2c(cn1)n(C)c(=O)n2C1CCCC1]

A degassed mixture of chloride 6 (127 mg, 0.50 mmol), 3-chloro-2-methylaniline (86 mg, 0.61 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-80%) of EtOAc/pet. ether, to give imidazopyridinone 41 (42 mg, 23%) as cream needles: mp (EtOAc/pet ether) 173-175° C.; ¹H NMR (CDCl₃) δ 7.81 (d, J=0.4 Hz, 1H, H-4), 7.31 (dd, J=7.5, 1.6 Hz, 1H, H-4′), 7.09-7.16 (m, 2H, H-5′, H-4′), 6.38 (s, 1H, H-7), 6.19 (br s, 1H, 6-NH), 4.74 (pent, J=8.7 Hz, 1H, 1-CH), 3.39 (s, 3H, 3-CH₃), 2.34 (s, 3H, 2′-CH₃), 1.90-2.02 (m, 4H, 2×CH₂), 1.75-1.83 (m, 2H, CH₂), 1.63-1.70 (m, 2H, CH₂); MS m/z 357.2 (MH⁺, 100%), 359.2 (MH⁺, 35%); HRMS calcd for C₁₉H₂₂ClN₄O (MH⁺) m/z 357.1477, found 357.1480 (−1.0 ppm). HPLC purity 98.0%.

### Example 36: SN39376 6-((4-Chloro-2-methylphenyl)amino)-1-cyclopentyl-3-methyl-1,3-dihydro-2H-imidazo[4,5-c]pyridin-2-one (42)

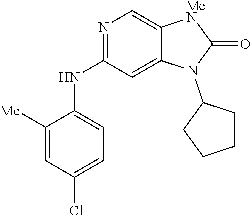 [SMILES: Cc1cc(Cl)ccc1Nc1cc2c(cn1)n(C)c(=O)n2C1CCCC1]

A degassed mixture of chloride 6 (126 mg, 0.50 mmol), 4-chloro-2-methylaniline (85 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 42 (85 mg, 48%) as a brown foam: ¹H NMR (CDCl₃) δ 7.81 (s, 1H, H-4), 7.36 (d, J=8.6 Hz, 1H, H-6′), 7.23 (d, J=2.4 Hz, 1H, H-3′), 7.15 (dd, J=8.6, 2.5 Hz, 1H, H-5′), 6.40 (s, 1H, H-7), 6.06 (br s, 1H, 6-NH), 4.73 (pent, J=8.8 Hz, 1H, 1-CH), 3.39 (s, 3H, 3-CH₃), 2.26 (s, 3H, 2′-CH₃), 1.93-2.02 (m, 4H, 2×CH₂), 1.78-1.88 (m, 2H, CH₂), 1.63-1.72 (m, 2H, CH₂); MS m/z 357.2 (MH⁺, 100%), 359.2 (MH⁺, 35%); HRMS calcd for C₁₉H₂₂ClN₄O (MH⁺) m/z 357.1477, found 357.1476 (0.1 ppm). HPLC purity 97.5%.

### Example 37: SN39379 6-((5-Chloro-2-methylphenyl)amino)-1-cyclopentyl-3-methyl-1,3-dihydro-2H-imidazo[4,5-c]pyridin-2-one (43)

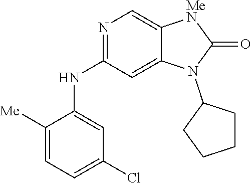 [SMILES: Cc1ccc(Cl)cc1Nc1cc2c(cn1)n(C)c(=O)n2C1CCCC1]

A degassed mixture of chloride 6 (131 mg, 0.52 mmol), 5-chloro-2-methylaniline (88 mg, 0.62 mmol), Pd₂dba₃ (24 mg, 26 μmol), XPhos (50 mg, 104 μmol) and Cs₂CO₃ (373 mg, 1.14 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-80%) of EtOAc/pet. ether, to give imidazopyridinone 43 (73 mg, 39%) as a tan foam: ¹H NMR (CDCl₃) δ 7.84 (s, 1H, H-4), 7.50 (d, J=2.1 Hz, 1H, H-6′), 7.14 (d, J=8.1 Hz, 1 H, H-3′), 6.94 (dd, J=8.1, 2.1 Hz, 1H, H-4′), 6.58 (s, 1H, H-7), 6.15 (br s, 1H, 6-NH), 4.81 (pent, J=8.8 Hz, 1H, 1-CH), 3.39 (s, 3H, 3-CH₃), 2.54 (s, 3H, 2′-CH₃), 1.83-2.08 (m, 6H, 3×CH₂), 1.66-1.75 (m, 2H, CH₂); MS m/z 357.2 (MH⁺, 100%), 359.2 (MH⁺, 35%); HRMS calcd for C₁₉H₂₂ClN₄O (MH⁺) m/z 357.1477, found 357.147180 (1.6 ppm). HPLC purity 98.0%.

### Example 38: SN39396 1-Cyclopentyl-3-methyl-6-((2-methyl-4-(methylsulfonyl)phenyl)amino)-1,3-dihydro-2H-imidazo[4,5-c]pyridin-2-one (44)

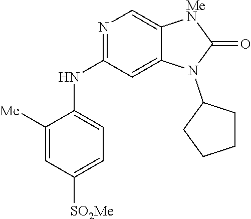 [SMILES: Cc1cc(S(C)(=O)=O)ccc1Nc1cc2c(cn1)n(C)c(=O)n2C1CCCC1]

A degassed mixture of chloride 6 (126 mg, 0.50 mmol), 2-methyl-4-(methylsulfonyl)aniline (110 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether, to give imidazopyridinone 44 (134 mg, 67%) as a tan powder: mp (EtOAc/pet ether) 201-203° C.; ¹H NMR (CDCl₃) δ 7.92 (d, J=0.6 Hz, 1H, H-4), 7.78 (d, J=8.5 Hz, 1H, H-6′), 7.69-7.75 (m, 2H, H-3′, H-5′), 6.70 (d, J=0.6 Hz, 1H, H-7), 6.40 (br s, 1H, 6-NH), 4.79 (pent, J=8.8 Hz, 1H, 1-CH), 3.44 (s, 3H, 3-CH₃), 3.04 (s, 3H, 4′-SO₂CH₃), 2.37 (s, 3H, 2′-CH₃), 2.00-2.08 (m, 4H, 2×CH₂), 1.87-1.96 (m, 2H, CH₂), 1.70-1.78 (m, 2H, CH₂); MS m/z 401.2 (MH⁺, 100%); HRMS calcd for C₂₀H₂₅N₄O₃S (MH⁺) m/z 401.1642, found 401.1635 (1.8 ppm). HPLC purity 98.7%.

### Example 39: SN39397 1-Cyclopentyl-3-methyl-6-((2-methyl-5-(methylsulfonyl)phenyl)amino)-1,3-dihydro-2H-imidazo[4,5-c]pyridin-2-one (45)

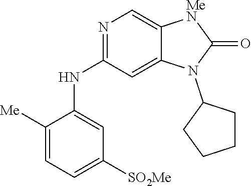 [SMILES: Cc1ccc(S(C)(=O)=O)cc1Nc1cc2c(cn1)n(C)c(=O)n2C1CCCC1]

A degassed mixture of chloride 6 (126 mg, 0.50 mmol), 2-methyl-5-(methylsulfonyl)aniline (111 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether, to give imidazopyridinone 45 (156 mg, 78%) as a tan powder: mp (EtOAc/pet ether) 170-172° C.; ¹H NMR (CDCl₃) δ 8.15 (d, J=1.8 Hz, 1H, H-6′), 7.88 (s, 1H, H-4), 7.46 (dd, J=7.8, 1.8 Hz, 1H, H-4′), 7.38 (d, J=7.8 Hz, 1H, H-3), 6.66 (d, J=0.6 Hz, 1H, H-7), 6.26 (br s, 1H, 6-NH), 4.82 (pent, J=8.8 Hz, 1H, 1-CH), 3.42 (s, 3H, 3-CH₃), 3.02 (s, 3H, 5′-SO₂CH₃), 2.38 (s, 3H, 2′-CH₃), 1.98-2.07 (m, 4H, 2×CH₂), 1.84-1.94 (m, 2H, CH₂), 1.65-1.75 (m, 2H, CH₂); MS m/z 401.2 (MH⁺, 100%); HRMS calcd for C₂₀H₂₅N₄O₃S (MH⁺) m/z 401.1642, found 401.1638 (1.0 ppm). HPLC purity 99.7%.

### Example 40: SN39398 4-((1-Cyclopentyl-3-methyl-2-oxo-2,3-dihydro-1H-imidazo[4,5-c]pyridin-6-yl)amino)-3-methylbenzonitrile (46)

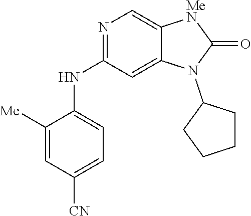 [SMILES: Cc1cc(C#N)ccc1Nc1cc2c(cn1)n(C)c(=O)n2C1CCCC1]

A degassed mixture of chloride 6 (129 mg, 0.51 mmol), 4-amino-3-methylbenzonitrile (81 mg, 0.61 mmol), Pd₂dba₃ (23 mg, 26 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-100% EtOAc/pet. ether) to give nitrile 50 (43 mg, 24%) as tan crystals: ¹H NMR (CDCl₃) δ 7.91 (d, J=0.4 Hz, 1H, H-4′), 7.70 (d, J=8.7 Hz, 1H, H-5), 7.43-7.47 (m, 2H, H-2, H-6), 6.67 (d, J=0.4 Hz, 1H, H-7), 6.36 (br s, 1H, 4-NH), 4.80 (pent, J=8.8 Hz, 1H, 1′-CH), 3.43 (s, 3H, 3′-CH₃), 2.32 (s, 3H, 3-CH₃), 1.99-2.08 (m, 4H, 2×CH₂), 1.86-1.96 (m, 2H, CH₂), 1.70-1.79 (m, 2H, CH₂); MS m/z 348.2 (MH⁺, 100%); HRMS calcd for C₂₀H₂₂N₅O (MH⁺) m/z 348.1819, found 348.1812 (2.0 ppm). HPLC purity 99.9%.

### Example 41: SN39401 4-((1-Cyclopentyl-3-methyl-2-oxo-2,3-dihydro-1H-imidazo[4,5-c]pyridin-6-yl)amino)-2-methylbenzonitrile (47)

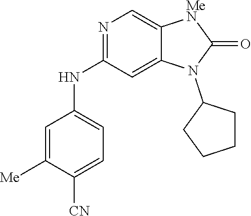 [SMILES: Cc1cc(Nc2cc3c(cn2)n(C)c(=O)n3C2CCCC2)ccc1C#N]

A degassed mixture of chloride 6 (129 mg, 0.51 mmol), 4-amino-2-methylbenzonitrile (81 mg, 0.62 mmol), Pd₂dba₃ (26 mg, 23 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-60% EtOAc/pet. ether) to give nitrile 47 (66 mg, 37%) as cream crystals: ¹H NMR (CDCl₃) δ 7.91 (s, 1H, H-4′), 7.49 (d, J=8.5 Hz, 1H, H-6), 7.29 (d, J=2.2 Hz, 1H, H-3), 7.20 (dd, J=8.5, 2.2 Hz, 1H, H-5), 6.71 (br s, 1H, 4-NH), 6.63 (s, 1H, H-7), 4.83 (pent, J=8.7 Hz, 1H, 1′-CH), 3.42 (s, 3H, 3′-CH₃), 2.50 (s, 3H, 2-CH₃), 1.99-2.09 (m, 4H, 2×CH₂), 1.86-1.95 (m, 2H, CH₂), 1.70-1.78 (m, 2H, CH₂); MS m/z 348.2 (MH⁺, 100%); HRMS calcd for C₂₀H₂₂N₅O (MH⁺) m/z 348.1819, found 348.1812 (2.0 ppm). HPLC purity 100.0%.

### Example 42: SN39369 1-Cyclopentyl-6-((4-methoxy-2-methylphenyl)amino)-3-methyl-1,3-dihydro-2H-imidazo[4,5-c]pyridin-2-one (48)

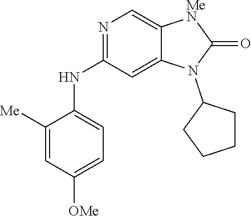 [SMILES: COc1ccc(Nc2cc3c(cn2)n(C)c(=O)n3C2CCCC2)c(C)c1]

A degassed mixture of chloride 6 (128 mg, 0.51 mmol), 4-methoxy-2-methylaniline (84 mg, 0.61 mmol), Pd₂dba₃ (23 mg, 25 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether, to give imidazopyridinone 48 (84 mg, 47%) as a tan gum: ¹H NMR (CDCl₃) δ 7.75 (d, J=0.4 Hz, 1H, H-4), 7.23 (d, J=8.6 Hz, 1H, H-6′), 6.84 (d, J=2.9 Hz, 1H, H-3′), 6.77 (d, J=8.6, 2.9 Hz, 1H, H-5′), 6.10 (s, 1H, H-7), 5.99 (br s, 1H, 6-NH), 4.67 (pent, J=8.7 Hz, 1H, 1-CH), 3.82 (s, 3H, 4′-OCH₃), 3.37 (s, 3H, 3-CH₃), 2.50 (s, 3H, 2′-CH₃), 1.87-1.97 (m, 4H, 2×CH₂), 1.70-1.80 (m, 2H, CH₂), 1.57-1.67 (m, 2H, CH₂); MS m/z 353.2 (MH⁺, 100%); HRMS HRMS calcd for C₂₀H₂₅N₄O₂ (MH⁺) m/z 353.1972, found 353.1979 (−2.0 ppm). HPLC purity 99.4%.

### Example 43: SN39382 1-Cyclopentyl-6-((5-methoxy-2-methylphenyl)amino)-3-methyl-1,3-dihydro-2H-imidazo[4,5-c]pyridin-2-one (49)

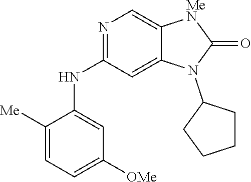 [SMILES: COc1ccc(C)c(Nc2cc3c(cn2)n(C)c(=O)n3C2CCCC2)c1]

A degassed mixture of chloride 6 (128 mg, 0.51 mmol), 5-methoxy-2-methylaniline (84 mg, 0.61 mmol), Pd₂dba₃ (23 mg, 26 μmol), XPhos (41 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 49 (93 mg, 52%) as a brown foam: ¹H NMR (CDCl₃) δ 7.82 (s, 1H, H-4), 7.13 (d, J=8.3 Hz, 1H, H-3′), 7.03 (d, J=2.6 Hz, 1H, H-6′), 6.57 (dd, J=8.3, 2.6 Hz, 1H, H-4′), 6.54 (d, J=0.6 Hz, 1H, H-7), 6.15 (br s, 1H, 6-NH), 4.76 (pent, J=8.8 Hz, 1H, 1-CH), 3.76 (s, 3H, 5′-OCH₃), 3.40 (s, 3H, 3-CH₃), 2.21 (s, 3H, 2′-CH₃), 1.96-2.04 (m, 4H, 2×CH₂), 1.78-1.88 (m, 2H, CH₂), 1.62-1.72 (m, 2H, CH₂); MS m/z 353.2 (MH⁺, 100%); HRMS calcd for C₂₀H₂₅N₄O₂ (MH⁺) m/z 353.1972, found 352.1964 (2.2 ppm). HPLC purity 99.0%.

### Example 44: SN39474 1-Cyclopentyl-6-((4-methoxy-2-methylphenyl)(methyl)amino)-3-methyl-1,3-dihydro-2H-imidazo[4,5-c]pyridin-2-one (50)

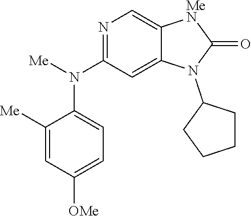 [SMILES: COc1ccc(N(C)c2cc3c(cn2)n(C)c(=O)n3C2CCCC2)c(C)c1]

NaH (60% dispersion, 13 mg, 0.33 mmol) was added to a stirred solution of imidazopyridinone 44 (105 mg, 0.30 mmol) and MeI (28 μL, 0.45 mmol) in dry DMF (5 mL) at 5° C. The mixture was stirred at 20° C. for 16 h and then quenched with ice/water (2 mL). The mixture was partitioned between EtOAc (50 mL) and water (30 ml). The organic fraction was washed with water (2×30 mL), washed with brine (30 mL), dried (MgSO₄), filtered and the solvent evaporated. The residue was purified by chromatography, eluting with a gradient (50-80%) of EtOAc/pet. ether, to give imidazopyridinone 50 (69 mg, 66%) as a clear gum: ¹H NMR (CDCl₃) δ 7.81 (s, 1H, H-4), 7.09 (d, J=8.5 Hz, 1H, H-6′), 6.86 (d, J=2.9 Hz, 1H, H-3′), 6.82 (d, J=8.5, 2.9 Hz, 1H, H-5′), 5.69 (s, 1H, H-7), 4.62 (pent, J=8.6 Hz, 1H, 1-CH), 3.83 (s, 3H, 4′-OCH₃), 3.36 (s, 3H, 6-NCH₃), 3.34 (s, 3H, 3-CH₃), 2.05 (s, 3H, 2′-CH₃), 1.76-1.90 (m, 4H, 2×CH₂), 1.50-1.60 (m, 4H, 2×CH₂); MS m/z 367.2 (MH⁺, 100%); HRMS calcd for C₂₁H₂₇N₄O₂ (MH⁺) m/z 367.2129, found 367.2124 (1.2 ppm). HPLC purity 97.2%.

### Example 45: SN39370 6-((4-(Benzyloxy)-2-methylphenyl)amino)-1-cyclopentyl-3-methyl-1,3-dihydro-2H-imidazo[4,5-c]pyridin-2-one (51)

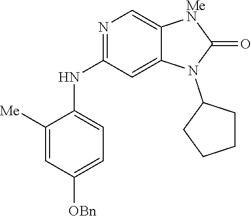 [SMILES: Cc1cc(OCc2ccccc2)ccc1Nc1cc2c(cn1)n(C)c(=O)n2C1CCCC1]

A degassed mixture of chloride 6 (281 mg, 1.12 mmol), 4-benzyoxy-2-methylaniline (286 mg, 1.34 mmol), Pd₂dba₃ (51 mg, 56 μmol), XPhos (107 mg, 224 μmol) and Cs₂CO₃ (803 mg, 2.46 mmol) in dioxane (10 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 51 (219 mg, 46%) as a tan foam: ¹H NMR (CDCl₃) δ 7.76 (s, 1H, H-4), 7.45 (br d, J=8.9 Hz, 2 H, H-2″, H-6″), 7.36-7.43 (m, 2H, H-3″, H-5″), 7.33 (br t, J=7.2 Hz, 1H, H-4″), 7.23 (d, J=8.6 Hz, 1H, H-6′), 6.92 (d, J=2.9 Hz, 1H, H-3′), 6.83 (dd, J=8.6, 2.9 Hz, 1H, H-5′), 6.12 (d, J=0.5 Hz, 1H, H-7), 5.98 (br s, 1H, 6-NH), 5.07 (s, 2H, CH₂O), 4.67 (pent, J=8.8 Hz, 1H, 1-CH), 3.37 (s, 3H, 3-CH₃), 2.24 (s, 3H, 2′-CH₃), 1.88-1.97 (m, 4H, 2×CH₂), 1.68-1.78 (m, 2H, CH₂), 1.57-1.65 (m, 2H, CH₂); MS m/z 429.2 (MH⁺, 100%); HRMS calcd for C₂₆H₂₉N₄O₂ (MH⁺) m/z 429.2285, found 429.2292 (−1.6 ppm). HPLC purity 99.0%.

### Example 46: SN39642 1-Cyclopentyl-6-((4-fluoro-2-methylphenyl)amino)-3-methyl-1,3-dihydro-2H-imidazo[4,5-c]pyridin-2-one (52)

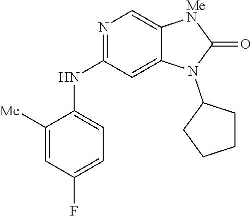 [SMILES: Cc1cc(F)ccc1Nc1cc2c(cn1)n(C)c(=O)n2C1CCCC1]

A degassed mixture of chloride 6 (404 mg, 1.61 mmol), 4-fluoro-2-methylaniline (241 mg, 1.93 mmol), Pd₂dba₃ (74 mg, 81 μmol), XPhos (154 mg, 154 μmol) and Cs₂CO₃ (1.154 mg, 3.54 mmol) in dioxane (20 mL) was stirred in a sealed tube at 120° C. for 16 h. The mixture was cooled, diluted with EtOAc (80 mL), filtered through diatomaceous earth and the filtrate was evaporated. The residue was partitioned between EtOAc (80 ml) and water (80 mL). The organic fraction was washed with water (50 mL), washed with brine (50 mL), dried (MgSO₄), filtered and the solvent evaporated. The residue was purified by chromatography, eluting with a gradient (50-100%) of EtOAc/pet. ether, to give imidazopyridinone 52 (368 mg, 67%) as a tan gum: ¹H NMR (CDCl₃) δ 7.77 (d, J=0.5 Hz, 1H, H-4), 7.31 (dd, J=8.7, 5.3 Hz, 1H, H-6′), 6.98 (dd, J=9.2, 2.9 Hz, 1H, H-3′), 6.92 (dt, J=8.4, 2.9 Hz, 1H, H-5′), 6.19 (d, J=0.6 Hz, 1H, H-7), 6.15 (br s, 1H, 6-NH), 4.70 (pent, J=8.7 Hz, 1H, 1-CH), 3.38 (s, 3H, 3-CH₃), 2.27 (s, 3H, 2′-CH₃), 1.88-2.00 (m, 4H, 2×CH₂), 1.73-1.82 (m, 2H, CH₂), 1.60-1.70 (m, 2H, CH₂); MS m/z 341.2 (MH⁺, 100%). HRMS calcd for C₁₉H₂₂FN₄O (MH⁺) m/z 341.1772, found 341.1776 (−1.1 ppm). HPLC purity 97.9%.

### Example 47: SN39748 1-Cyclopentyl-3-methyl-6-((2-methyl-4-(trifluoromethoxy)phenyl)amino)-1,3-dihydro-2H-imidazo[4,5-c]pyridin-2-one (53)

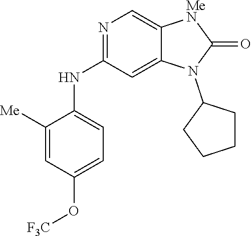 [SMILES: Cc1cc(OC(F)(F)F)ccc1Nc1cc2c(cn1)n(C)c(=O)n2C1CCCC1]

A degassed mixture of chloride 6 (136 mg, 0.54 mmol), 2-methyl-4-(trifluoromethoxy)aniline (124 mg, 0.65 mmol), Pd₂dba₃ (25 mg, 27 μmol), XPhos (51 mg, 108 μmol) and Cs₂CO₃ (387 mg, 1.19 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether, to give imidazopyridinone 53 (96 mg, 44%) as a tan gum: ¹H NMR (CDCl₃) δ 7.84 (s, 1H, H-4), 7.46 (d, J=8.7 Hz, 1H, H-6′), 7.13 (br s, 1H, H-3′), 7.08 (br d, J=8.6 Hz, 1H, H-5′), 6.40 (d, J=0.3 Hz, 1H, H-7), 6.10 (br s, 1H, 6-NH), 4.77 (pent, J=8.8 Hz, 1H, 1-CH), 3.41 (s, 3H, 3-CH₃), 2.31 (s, 3H, 2′-CH₃), 1.94-2.04 (m, 4H, 2×CH₂), 1.78-1.87 (m, 2H, CH₂), 1.66-1.76 (m, 2H, CH₂); MS m/z 407.2 (MH⁺, 100%). HRMS calcd for C₂₀H₂₂F₃N₄O₂ (MH⁺) m/z 407.1689, found 407.1692 (−0.5 ppm). HPLC purity 98.2%.

### Example 48: SN3976 1-Cyclopentyl-6-((4-(difluoromethoxy)-2-methylphenyl)amino)-3-methyl-1,3-dihydro-2H-imidazo[4,5-c]pyridin-2-one (54)

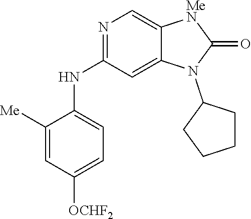 [SMILES: Cc1cc(OC(F)F)ccc1Nc1cc2c(cn1)n(C)c(=O)n2C1CCCC1]

A degassed mixture of chloride 6 (136 mg, 0.54 mmol), 4-(difluoromethoxy)-2-methylaniline. HCl (136 mg, 0.65 mmol), Pd₂dba₃ (25 mg, 27 μmol), XPhos (52 mg, 108 μmol) and Cs₂CO₃ (563 mg, 1.73 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether, to give imidazopyridinone 54 (105 mg, 50%) as a tan gum: ¹H NMR (CDCl₃) δ 7.80 (s, 1H, H-4), 7.39 (d, J=8.7 Hz, 1H, H-6), 7.03 (d, J=2.7 Hz, 1H, H-3′), 6.98 (dd, J=8.7, 2.7 Hz, 1H, H-5′), 6.48 (t, J=74.2 Hz, 1H, 4′-OCHF₂), 6.33 (d, J=0.6 Hz, 1H, H-7), 6.07 (br s, 1H, 6-NH), 4.73 (pent, J=8.7 Hz, 1H, 1-CH), 3.39 (s, 3H, 3-CH₃), 2.28 (s, 3H, 2′-CH₃), 1.98-2.04 (m, 4H, 2×CH₂), 1.73-1.84 (m, 2H, CH₂), 1.62-1.70 (m, 2H, CH₂); MS m/z 389.2 (MH⁺, 100%). HRMS calcd for C₂₀H₂₃F₂N₄O₂ (MH⁺) m/z 389.1784, found 389.1783 (0.1 ppm). HPLC purity 99.9%.

### Example 49: SN39764 1-Cyclopentyl-6-((4-ethoxy-2-methylphenyl)amino)-3-methyl-1,3-dihydro-2H-imidazo[4,5-c]pyridin-2-one (55)

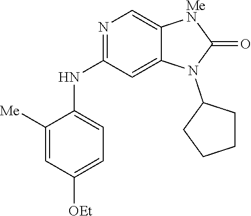 [SMILES: CCOc1ccc(Nc2cc3c(cn2)n(C)c(=O)n3C2CCCC2)c(C)c1]

4-Ethoxy-2-methyl-1-nitrobenzene. Diethyl sulfate (0.94 mL, 7.2 mmol) was added to a stirred suspension of 3-methyl-4-nitrophenol (1.00 g, 6.5 mmol) and K₂CO₃ (2.26 g, 16.3 mmol) in methyl ethyl ketone (50 mL) and the mixture was stirred at 70° C. for 4 h. The mixture was cooled to 20° C. and cNH₄OH solution (2 mL) added and the mixture stirred at 20° C. for 16 h. The mixture was partitioned between EtOAc (100 mL) and water (100 ml). The organic fraction was washed with water (2×50 mL), washed with brine (50 mL), dried (MgSO₄), filtered and the solvent evaporated. The residue was purified by chromatography, eluting with a gradient (5-10%) of EtOAc/pet. ether, to give nitrobenzene (1.14 g, 96%) as a white solid: mp 50-51° C. (lit.¹ mp 50-52° C.); ¹H NMR (CDCl₃) δ 8.08 (d, J=8.6 Hz, 1H, H-6), 6.76-6.80 (m, 2H, H-3, H-5), 4.10 (q, J=7.0 Hz, 2 H, CH₂O), 2.63 (s, 3H, 2-CH₃), 1.45 (t, J=7.0 Hz, 3 H, CH₃); MS m/z 182.2 (MH⁺, 100%). 4-Ethoxy-2-methylaniline. A mixture of nitrobenzene (413 mg, 2.3 mmol) and Pd/C (5%, 50 mg) in EtOH (100 mL) was stirred vigorously under H2 (50 psi) for 6 h. The mixture was filtered through diatomaceous earth and the pad washed with EtOH (10 mL). The solvent was evaporated to give the aniline (329 mg, 95%) as a clear oil: ¹H NMR (CDCl₃) δ 6.35-6.68 (m, 1H, H-6), 6.60-6.62 (m, 2H, H-3, H-5), 3.95 (q, J=7.0 Hz, 2 H, CH₂O), 3.34 (br s, 2H, NH₂), 2.16 (s, 3H, 2-CH₃), 1.36 (t, J=7.0 Hz, 3 H, CH₃); MS m/z 152.2 (MH⁺, 100%). 1-Cyclopentyl-6-((4-ethoxy-2-methylphenyl)amino)-3-methyl-1,3-dihydro-2H-imidazo[4,5-c]pyridin-2-one (55). A degassed mixture of chloride 6 (127 mg, 0.50 mmol), 4-ethoxy-2-methylaniline (92 mg, 0.61 mmol), Pd₂dba₃ (23 mg, 25 μmol), XPhos (48 mg, 100 μmol) and Cs₂CO₃ (358 mg, 1.10 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 55 (123 mg, 67%) as a tan foam: mp 192-194° C.; ¹H NMR (CDCl₃) δ 7.75 (d, J=0.3 Hz, 1H, H-4), 7.21 (d, J=8.6 Hz, 1H, H-6′), 6.83 (d, J=2.9 Hz, 1H, H-3′), 6.76 (dd, J=8.6, 2.9 Hz, 1H, H-5′), 6.10 (d, J=0.5 Hz, 1H, H-7), 5.99 (br s, 1H, 6-NH), 4.67 (p, J=8.7 Hz, 1H, 1-CH), 4.04 (q, J=7.0 Hz, 2 H, 4′-OCH₂), 3.37 (s, 3H, 3-CH₃), 2.24 (s, 3H, 2′-CH₃), 1.90-1.96 (m, 4H, 2×CH₂), 1.70-1.80 (m, 2H, CH₂), 1.57-1.66 (m, 2H, CH₂), 1.42 (t, J=7.0 Hz, 3 H, CH₃); MS m/z 367.2 (MH⁺, 100%). HRMS calcd for C₂₁H₂₇N₄O₂ (MH⁺) m/z 367.2129, found 367.2141 (−3.5 ppm). HPLC purity 98.2%.

### Example 50: SN39673 1-Cyclopentyl-6-((4-((2-(dimethylamino)ethyl)amino)-2-methylphenyl)amino)-3-methyl-1,3-dihydro-2H-imidazo[4,5-c]pyridin-2-one (56)

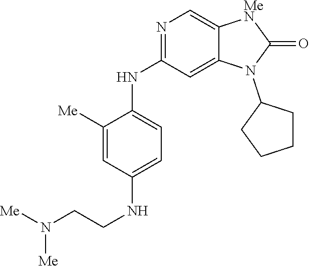 [SMILES: Cc1cc(NCCN(C)C)ccc1Nc1cc2c(cn1)n(C)c(=O)n2C1CCCC1]

N,N-Dimethyl-2-(3-methyl-4-nitrophenoxy)ethan-1-amine. A mixture of 4-fluoro-2-methyl-1-nitrobenzene (2.00 g, 12.9 mmol), K₂CO₃ (5.35 g, 38.7 mmol) and 2-(dimethylamino)ethan-1-ol (1.61 g, 18.1 mmol) in DMF (30 mL) was stirred at 60° C. for 16 h. The mixture was partitioned between EtOAc (200 mL) and water (100 mL). The organic fraction was washed with water (3×100 mL), brine (50 mL) and dried (MgSO₄) and the solvent was evaporated. The residue was purified by chromatography, eluting with a gradient (0-5%) of MeOH/DCM, to give amine (0.58 g, 20%) as a red oil: ¹H NMR (CDCl₃) δ 8.07 (dd, J=7.8, 1.7 Hz, 1H, H-6′), 6.79-6.83 (m, 2H, H-2′, H-5′), 4.13 (br t, J=5.6 Hz, 2 H, H-2), 2.76 (br t, J=5.6 Hz, 2 H, H-1), 2.62 (s, 3H, 3′-CH₃), 2.36 [s, 6 H, N(CH₃)₂]; MS m/z 225.1 (MH⁺, 100%). 4-(2-(Dimethylamino)ethoxy)-2-methylaniline. A mixture of nitroaniline (0.58 g, 2.59 mmol) and Pd/C (100 mg) in EtOH (100 mL) and EtOAc (100 mL) was stirred under Hz (60 psi) for 4 h. The mixture was filtered through a pad of diatomaceous earth, washed with EtOH (50 mL) and the solvent evaporated to give the aniline (0.44 g, 87%) as a beige solid: ¹H NMR [(CD₃)₂SO] δ 6.56 (d, J=1.2 Hz, 1H, H-3), 6.48-6.53 (m, 2H, H-5, H-6), 4.35 (br s, 2H, 1-NH₂), 3.84 (br t, J=6.0 Hz, 2 H, H-1′), 2.53 (br t, J=6.0 Hz, 2 H, H-2′), 2.18 [s, 6 H, N(CH₃)_{2], 2.02} (s, 3H, 2-CH₃); MS m/z 195.1 (MH⁺, 100%).

1-Cyclopentyl-6-((4-((2-(dimethylamino)ethyl)amino)-2-methylphenyl)amino)-3-methyl-1,3-dihydro-2H-imidazo[4,5-c]pyridin-2-one (56). A degassed mixture of chloride 6 (148 mg, 0.59 mmol), 4-(2-(dimethylamino)ethoxy)-2-methylaniline (91 mg, 0.47 mmol), Pd₂dba₃ (27 mg, 30 μmol), XPhos (56 mg, 118 μmol) and Cs₂CO₃ (432 mg, 1.30 mmol) in MeCN (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/DCM, to give imidazopyridinone 56 (80 mg, 33%) as a tan powder: ¹H NMR (CDCl₃) δ 7.74 (s, 1H, H-4), 7.23 (d, J=8.6 Hz, 1H, H-6′), 6.86 (d, J=2.9 Hz, 1H, H-3′), 6.78 (d, J=8.6, 2.9 Hz, 1H, H-5′), 6.16 (br s, 1H, 6-NH), 6.10 (s, 1H, H-7), 4.67 (pent, J=8.7 Hz, 1H, 1-CH), 4.10 (br t, J=5.6 Hz, 2 H, CH₂O), 3.37 (s, 3H, 3-CH₃), 2.80 (br t, J=5.6, 2 H, CH₂N), 2.40 [s, 6 H, N(CH₃)_{2], 2.14} (s, 3H, 2′-CH₃), 1.90-1.98 (m, 4H, 2×CH₂), 1.70-1.80 (m, 2H, CH₂), 1.60-1.66 (m, 2H, CH₂); MS m/z 410.2 (MH⁺, 100%). HRMS calcd for C₂₃H₃₂N₅O₂ (MH⁺) m/z 410.2551, found 410.2557 (−1.7 ppm). HPLC purity 97.4%.

### Example 51: SN39663 1-Cyclopentyl-3-methyl-6-((2-methyl-4-morpholinophenyl)amino)-1,3-dihydro-2H-imidazo[4,5-c]pyridin-2-one (57)

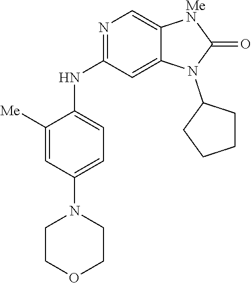 [SMILES: Cc1cc(N2CCOCC2)ccc1Nc1cc2c(cn1)n(C)c(=O)n2C1CCCC1]

4-(3-Methyl-4-nitrophenyl)morpholine. A mixture of 4-fluoro-2-methyl-1-nitrobenzene (1.92 g, 12.4 mmol), K₂CO₃ (2.05 g, 14.9 mmol) and morpholine (2.16 g, 24.8 mmol) in DMF (20 mL) was stirred at 60° C. for 72 h. The mixture was partitioned between EtOAc (200 mL) and water (100 mL). The organic fraction was washed with water (3×100 mL), brine (50 mL) and dried (MgSO₄) and the solvent was evaporated. The residue was purified by chromatography, eluting with 20% EtOAc/pet. ether, to give morpholide (2.40 g, 87%) as yellow needles: mp 140-142° C.; ¹H NMR (CDCl₃) δ 8.09 (d, J=9.2 Hz, 1H, H-5′), 6.71 (dd, J=9.2, 2.9 Hz, 1H, H-6′), 6.65 (d, J=2.9 Hz, 1H, H-2′), 3.86 (br dd, J=5.0, 4.9 Hz, 4 H, H-2, H-6), 3.34 (br dd, J=5.0, 4.9 Hz, 4 H, H-3, H-5), 2.64 (s, 3H, 3′-CH₃); MS m/z 223.1 (MH⁺, 100%).

2-Methyl-4-morpholinoaniline. A mixture of nitroaniline (1.30 g, 5.85 mmol) and Pd/C (100 mg) in EtOH (100 mL) and EtOAc (100 mL) was stirred under Hz (60 psi) for 4 h. The mixture was filtered through a pad of diatomaceous earth, washed with EtOH (50 mL) and the solvent evaporated to give the aniline (1.12 g, 99%) as a beige solid: mp 84-86° C.; ¹H NMR (CDCl₃) δ 6.71 (d, J=2.6 Hz, 1H, H-3), 6.67 (dd, J=8.4, 2.6 Hz, 1H, H-5), 6.63 (d, J=8.4 Hz, 1H, H-6), 3.84 (br dd, J=4.8, 4.7 Hz, 4 H, H-2′, H-6′), 3.38 (br s, 2H, NH₂), 3.34 (br dd, J=4.8, 4.7 Hz, 4 H, H-3′, H-5′), 2.64 (s, 3H, 3-CH₃); MS m/z 223.1 (MH⁺, 100%).

1-Cyclopentyl-3-methyl-6-((2-methyl-4-morpholinophenyl)amino)-1,3-dihydro-2H-imidazo[4,5-c]pyridin-2-one (57). A degassed mixture of chloride 6 (133 mg, 0.53 mmol), 2-methyl-4-morpholinoaniline (122 mg, 0.63 mmol), Pd₂dba₃ (24 mg, 27 μmol), XPhos (51 mg, 106 μmol) and Cs₂CO₃ (380 mg, 1.17 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether, to give imidazopyridinone 57 (110 mg, 51%) as a tan powder: ¹H NMR (CDCl₃) δ 7.75 (d, J=0.5 Hz, 1H, H-4), 7.23 (d, J=8.6 Hz, 1H, H-6′), 6.84 (d, J=2.8 Hz, 1H, H-3′), 6.78 (d, J=8.6, 2.9 Hz, 1H, H-5′), 6.15 (d, J=0.6 Hz, 1H, H-7), 6.07 (br s, 1H, 6-NH), 4.66 (pent, J=8.7 Hz, 1H, 1-CH), 3.88 (br dd, J=4.9, 4.7 Hz, 4 H, H-2″, H-6″), 3.47 (s, 3H, 3-CH₃), 3.14 (br dd, J=4.9, 4.7 Hz, 4 H, H-3″, H-5″), 2.25 (s, 3H, 2′-CH₃), 1.90-1.98 (m, 4H, 2×CH₂), 1.72-1.82 (m, 2H, CH₂), 1.55-1.65 (m, 2H, CH₂); MS m/z 408.2 (MH⁺, 100%). HRMS HRMS calcd for C₂₃H₃₀N₅O₂ (MH⁺) m/z 408.2394, found 408.2396 (−0.4 ppm). HPLC purity 98.1%.

### Example 52: SN39637 1-Cyclopentyl-3-methyl-6-((2-methyl-4-(4-methylpiperazin-1-yl)phenyl)amino)-1,3-dihydro-2H-imidazo[4,5-c]pyridin-2-one (58)

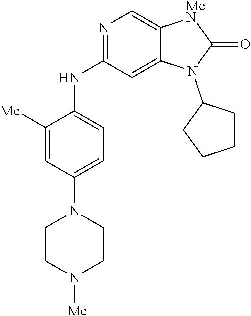 [SMILES: Cc1cc(N2CCN(C)CC2)ccc1Nc1cc2c(cn1)n(C)c(=O)n2C1CCCC1]

1-Methyl-4-(3-methyl-4-nitrophenyl)piperazine. A mixture of 4-fluoro-2-methyl-1-nitrobenzene (2.28 g, 14.7 mmol), K₂CO₃ (4.06 g, 29.4 mmol) and 1-methylpiperazine (2.20 g, 22.1 mmol) in DMF (50 mL) was stirred at 80° C. for 16 h. The mixture was partitioned between EtOAc (200 mL) and water (100 mL). The organic fraction was washed with water (3×100 mL), brine (50 mL) and dried (MgSO₄) and the solvent was evaporated. The residue was purified by chromatography, eluting with a gradient (0-5%) of MeH/DCM, to give piperazine (3.22 g, 93%) as yellow plates: mp 63-65° C.; ¹H NMR (CDCl₃) δ 8.08 (d, J=9.3 Hz, 1H, H-5′), 6.70 (dd, J=9.3, 2.8 Hz, 1H, H-6′), 6.64 (d, J=2.8 Hz, 1H, H-2′), 3.04 (br dd, J=5.2, 5.1 Hz, 4 H, H-3, H-5), 2.63 (s, 3H, 1-CH₃), 2.55 (br dd, J=5.2, 5.1 Hz, 4 H, H-2, H-6), 2.35 (s, 3H, 3′-CH₃); MS m/z 236.1 (MH⁺, 100%).

2-Methyl-4-(4-methylpiperazin-1-yl)aniline. A mixture of nitroaniline (0.42 g, 1.79 mmol) and Pd/C (50 mg) in EtOH (50 mL) and EtOAc (50 mL) was stirred under Hz (50 psi) for 4 h. The mixture was filtered through a pad of diatomaceous earth, washed with EtOH (50 mL) and the solvent evaporated to give the aniline (0.35 g, 96%) as a white solid: mp 100-102° C.; 1H NMR (CDCl₃) δ 6.73 (d, J=2.7 Hz, 1H, H-3), 6.69 (dd, J=8.4, 2.7 Hz, 1H, H-5), 6.62 (d, J=8.4 Hz, 1H, H-6), 3.37 (br s, 2H, NH₂), 3.07 (br dd, J=5.0, 4.9 Hz, 4 H, H-3′, H-5′), 2.57 (br dd, J=5.0, 4.9 Hz, 4 H, H-2′, H-6′), 2.34 (s, 3H, 4′-CH₃), 2.16 (s, 3H, 3-CH₃); MS m/z 206.1 (MH⁺, 100%).

1-Cyclopentyl-3-methyl-6-((2-methyl-4-(4-methylpiperazin-1-yl)phenyl)amino)-1,3-dihydro-2H-imidazo[4,5-c]pyridin-2-one (58). A degassed mixture of chloride 6 (136 mg, 0.54 mmol), 2-methyl-4-(4-methylpiperazin-1-yl)aniline (133 mg, 0.65 mmol), Pd₂dba₃ (25 mg, 27 μmol), XPhos (51 mg, 108 μmol) and Cs₂CO₃ (387 mg, 1.19 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-5%) of MeOH/DCM, to give imidazopyridinone 58 (167 mg, 67%) as a tan gum: ¹H NMR (CDCl₃) δ 7.75 (s, 1H, H-4), 7.21 (d, J=8.6 Hz, 1H, H-6′), 6.86 (d, J=2.7 Hz, 1H, H-3′), 6.80 (d, J=8.6, 2.7 Hz, 1H, H-5′), 6.13 (d, J=0.5 Hz, 1H, H-7), 6.02 (br s, 1H, 6-NH), 4.65 (pent, J=8.7 Hz, 1H, 1-CH), 3.37 (s, 3H, 3-CH₃), 3.21 (br dd, J=5.1, 4.9 Hz, 4 H, H-3″, H-5″), 2.61 (br dd, J=5.1, 4.9 Hz, 4 H, H-2″, H-6″), 2.37 (s, 3H, 4′-CH₃), 2.23 (s, 3H, 2′-CH₃), 1.89-1.97 (m, 4H, 2×CH₂), 1.71-1.80 (m, 2H, CH₂), 1.57-1.67 (m, 2H, CH₂); MS m/z 421.2 (MH⁺, 100%); HRMS calcd for C₂₄H₃₃N₆O (MH⁺) m/z 421.2710, found 421.2718 (−1.7 ppm). HPLC purity 99.2%.

### Example 53: SN39758 1-Cyclopentyl-6-((4-methoxy-2-(trifluoromethyl)phenyl)amino)-3-methyl-1,3-dihydro-2H-imidazo[4,5-c]pyridin-2-one (59)

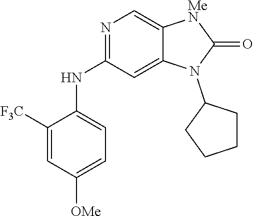 [SMILES: COc1ccc(Nc2cc3c(cn2)n(C)c(=O)n3C2CCCC2)c(C(F)(F)F)c1]

A degassed mixture of chloride 6 (137 mg, 0.54 mmol), 4-methoxy-2-(trifluoromethyl)aniline (124 mg, 0.65 mmol), Pd₂dba₃ (25 mg, 27 μmol), XPhos (51 mg, 108 μmol) and Cs₂CO₃ (387 mg, 1.19 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 59 (101 mg, 46%) as a tan gum: ¹H NMR (CDCl₃) δ 7.80 (s, 1H, H-4), 7.58 (d, J=8.9 Hz, 1H, H-6′), 7.18 (d, J=2.9 Hz, 1H, H-3′), 7.06 (dd, J=8.9, 2.9 Hz, 1H, H-5′), 6.34 (s, 1H, H-7), 6.31 (br s, 1H, 6-NH), 4.71 (pent, J=8.8 Hz, 1H, 1-CH), 3.85 (s, 3H, 4′-OCH₃), 3.38 (s, 3H, 3-CH₃), 1.94-2.02 (m, 4H, 2×CH₂), 1.78-1.88 (m, 2H, CH₂), 1.63-1.72 (m, 2H, CH₂); MS m/z 407.2 (MH⁺, 100%). HRMS calcd for C₂₀H₂₂F₃N₄O₂ (MH⁺) m/z 407.1689, found 407.1689 (0.1 ppm). HPLC purity 99.6%.

### Example 54: SN39762 2-((1-Cyclopentyl-3-methyl-2-oxo-2,3-dihydro-1H-imidazo[4,5-c]pyridin-6-yl)amino)-5-methoxybenzonitrile (60)

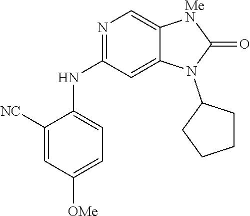 [SMILES: COc1ccc(Nc2cc3c(cn2)n(C)c(=O)n3C2CCCC2)c(C#N)c1]

A degassed mixture of chloride 6 (128 mg, 0.51 mmol), 2-amino-5-methoxybenzonitrile (90 mg, 0.61 mmol), Pd₂dba₃ (23 mg, 26 μmol), XPhos (49 mg, 102 μmol) and Cs₂CO₃ (366 mg, 1.12 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 60 (85 mg, 46%) as a yellow powder: mp (EtOAc/pet ether) 197-200° C.; ¹H NMR [(CD₃)₂SO] δ 9.16 (br s, 1H, 6′-NH), 8.81 (s, 1H, H-4′), 7.92 (d, J=2.8 Hz, 1H, H-3), 7.38 (d, J=8.9 Hz, 1H, H-6), 7.25 (dd, J=8.9, 2.8 Hz, 1H, H-5), 6.75 (s, 1H, H-7), 4.72 (pent, J=8.5 Hz, 1H, 1′-CH), 3.87 (s, 3H, 4-OCH₃), 3.30 (s, 3H, 3′-CH₃), 2.00-2.08 (m, 4H, 2×CH₂), 1.86-1.95 (m, 2H, CH₂), 1.60-1.70 (m, 2H, CH₂); MS m/z 364.2 (MH⁺, 100%). HRMS calcd for C₂₀H₂₂N₅O₂ (MH⁺) m/z 364.1768, found 364.1766 (0.5 ppm). HPLC purity 98.8%.

### Example 55: SN39759 6-((2-Chloro-4-methoxyphenyl)amino)-1-cyclopentyl-3-methyl-1,3-dihydro-2H-imidazo[4,5-c]pyridin-2-one (61)

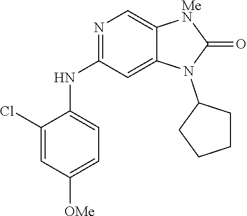 [SMILES: COc1ccc(Nc2cc3c(cn2)n(C)c(=O)n3C2CCCC2)c(Cl)c1]

A degassed mixture of chloride 6 (130 mg, 0.52 mmol), 2-chloro-4-methoxyaniline (98 mg, 0.62 mmol), Pd₂dba₃ (24 mg, 26 μmol), XPhos (50 mg, 104 μmol) and Cs₂CO₃ (373 mg, 1.14 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with 50% EtOAc/pet. ether, to give imidazopyridinone 61 (108 mg, 56%) as a grey foam: ¹H NMR (CDCl₃) δ 7.82 (d, J=0.3 Hz, 1H, H-4), 7.67 (d, J=9.0 Hz, 1H, H-6′), 7.00 (d, J=2.9 Hz, 1 H, H-3′), 6.83 (dd, J=9.0, 2.9 Hz, 1H, H-5′), 6.40 (br s, 2H, 6-NH, H-7), 4.75 (p, J=8.8 Hz, 1 H, 1-CH), 3.81 (s, 3H, 4′-OCH₃), 3.39 (s, 3H, 3-CH₃), 1.95-2.05 (m, 4H, 2×CH₂), 1.81-1.91 (m, 2H, CH₂), 1.64-1.72 (m, 2H, CH₂); MS m/z 373.2 (MH⁺, 100%). HRMS calcd for C₁₉H₂₂³⁵ClN₄O₂ (MH⁺) m/z 373.1426, found 373.1426 (−0.5 ppm); calcd for C₁₉H₂₂³⁷ClN₄O₂ (MH⁺) m/z 375.1403, found 375.1407 (−1.0 ppm). HPLC purity 99.3%.

### Example 56: SN39717 1-Cyclopentyl-6-((2,4-dimethoxyphenyl)amino)-3-methyl-1,3-dihydro-2H-imidazo[4,5-c]pyridin-2-one (62)

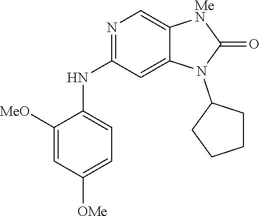 [SMILES: COc1ccc(Nc2cc3c(cn2)n(C)c(=O)n3C2CCCC2)c(OC)c1]

A degassed mixture of chloride 6 (1330 mg, 0.532 mmol), 2,4-dimethoxyaniline (97 mg, 0.63 mmol), Pd₂dba₃ (24 mg, 27 μmol), XPhos (51 mg, 106 μmol) and Cs₂CO₃ (380 mg, 1.17 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with a gradient (80-100%) of EtOAc/pet. ether, to give imidazopyridinone 62 (162 mg, 83%) as a pink foam: ¹H NMR (CDCl₃) δ 7.80 (d, J=0.5 Hz, 1H, H-4), 7.64 (d, J=8.7 Hz, 1H, H-6′), 6.55 (d, J=2.7 Hz, 1H, H-3′), 6.50 (dd, J=8.7, 2.7 Hz, 1H, H-5′), 6.46 (br s, 1H, 6-NH), 6.43 (d, J=0.6 Hz, 1H, H-7), 4.76 (p, J=8.8 Hz, 1H, 1-CH), 3.85 (s, 3H, 2′-OCH₃), 3.82 (s, 3H, 4′-OCH₃), 3.54 (s, 3H, 3-CH₃), 1.96-2.03 (m, 4H, 2×CH₂), 1.81-1.91 (m, 2H, CH₂), 1.64-1.72 (m, 2H, CH₂); MS m/z 369.2 (MH⁺, 100%). HRMS calcd for C₂₀H₂₅N₄O₃ (MH⁺) m/z 369.1921, found 369.1926 (−0.2 ppm). HPLC purity 99.6%.

### Example 57: SN39313 1-Cyclopentyl-3-methyl-6-(quinolin-6-ylamino)-1,3-dihydro-2H-imidazo[4,5-c]pyridin-2-one (63)

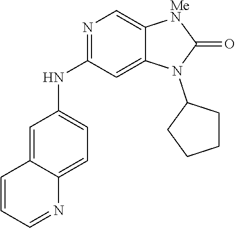 [SMILES: Cn1c(=O)n(C2CCCC2)c2cc(Nc3ccc4ncccc4c3)ncc21]

A degassed mixture of chloride 6 (121 mg, 0.48 mmol), quinolin-6-amine (83 mg, 0.58 mmol), Pd₂dba₃ (22 mg, 24 μmol), XPhos (46 mg, 96 μmol) and Cs₂CO₃ (344 mg, 1.06 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 63 (126 mg, 73%) as a yellow powder: mp (EtOAc/pet ether) 260-263° C.; ¹H NMR (CDCl₃) δ 8.76 (dd, J=4.2, 1.6 Hz, 1H, H-2′), 7.98-8.05 (m, 2H, H-4′, H-8′), 7.92 (s, 1H, H-4), 7.87 (d, J=2.5 Hz, 1H, H-5′), 7.56 (dd, J=9.1, 2.5 Hz, 1H, H-7), 7.34 (dd, J=8.3, 4.2 Hz, 1H, H-3′), 6.81 (br s, 1H, 6-NH), 6.72 (s, 1H, H-7), 4.82 (pent, J=8.8 Hz, 1H, 1-CH), 3.43 (s, 3H, 3-CH₃), 1.97-2.08 (m, 4H, 2×CH₂), 1.82-1.92 (m, 2H, CH₂), 1.65-1.75 (m, 2H, CH₂); MS m/z 360.2 (MH⁺, 100%). Anal calcd for C₂₁H₂₁N₅O·¼H₂O: C, 69.31; H, 5.95; N, 19.24. Found: C, 69.06; H, 5.62; N, 19.38%. HPLC purity 100.0%.

### Example 58: SN39316 1-Cyclopentyl-3-methyl-6-(quinolin-3-ylamino)-1,3-dihydro-2H-imidazo[4,5-c]pyridin-2-one (64)

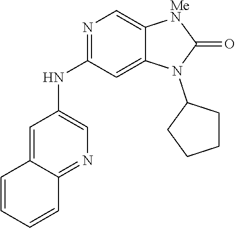 [SMILES: Cn1c(=O)n(C2CCCC2)c2cc(Nc3cnc4ccccc4c3)ncc21]

A degassed mixture of chloride 6 (126 mg, 0.50 mmol), quinolin-3-amine (87 mg, 0.60 mmol), Pd₂dba₃ (24 mg, 25 μmol), XPhos (47 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 64 (143 mg, 79%) as a tan powder: mp (EtOAc/pet ether) 207-210° C.; 1H NMR (CDCl₃) δ 8.80 (d, J=2.7 Hz, 1H, H-2′), 8.40 (d, J=2.6 Hz, 1H, H-4′), 8.02 (d, J=8.2 Hz, 1H, H-5′), 7.92 (s, 1H, H-4), 7.72 (dd, J=8.3, 1.5 Hz, 1H, H-8′), 7.56 (dd, J=8.3, 1.5 Hz, 1H, H-7′), 7.50 (ddd, J=8.2, 6.9, 1.3 Hz, 1H, H-6′), 6.83 (br s, 1H, 6-NH), 6.62 (d, J=0.5 Hz, 1H, H-7), 4.80 (pent, J=8.8 Hz, 1H, 1-CH), 3.43 (s, 3H, 3-CH₃), 1.98-2.08 (m, 4H, 2×CH₂), 1.84-1.94 (m, 2H, CH₂), 1.66-1.76 (m, 2H, CH₂); MS m/z 360.2 (MH⁺, 100%); HRMS calcd for C₂₁H₂₂N₅O (MH⁺) m/z 360.1819, found 360.1822 (−0.9 ppm). HPLC purity 99.9%.

### Example 59: SN39322 1-Cyclopentyl-3-methyl-6-(quinolin-5-ylamino)-1,3-dihydro-2H-imidazo[4,5-c]pyridin-2-one (65)

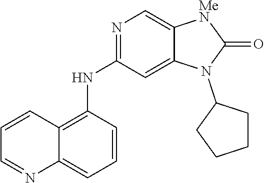 [SMILES: Cn1c(=O)n(C2CCCC2)c2cc(Nc3cccc4ncccc34)ncc21]

A degassed mixture of chloride 6 (120 mg, 0.48 mmol), quinolin-5-amine (83 mg, 0.57 mmol), Pd₂dba₃ (22 mg, 24 μmol), XPhos (46 mg, 96 μmol) and Cs₂CO₃ (344 mg, 1.06 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 65 (94 mg, 55%) as tan crystals: mp (EtOAc/pet ether) 150-152° C.; 1H NMR (CDCl₃) δ 8.95 (dd, J=4.2, 1.6 Hz, 1H, H-2′), 8.40 (ddd, J=8.5, 1.4, 0.8 Hz, 1H, H-4′), 7.94 (d, J=8.5 Hz, 1H, H-6′), 7.82 (s, 1H, H-4), 7.71 (dd, J=8.4, 7.6 Hz, 1H, H-7), 7.57 (d, J=7.4 Hz, 1H, H-8′), 7.40 (dd, J=8.5, 4.2 Hz, 1H, H-3′), 6.85 (br s, 1H, 6-NH), 6.36 (s, 1H, H-7), 4.68 (pent, J=8.8 Hz, 1H, 1-CH), 3.40 (s, 3H, 3-CH₃), 1.79-1.96 (m, 4H, 2×CH₂), 1.52-1.63 (m, 4H, 2×CH₂); MS m/z 360.2 (MH⁺, 100%); HRMS calcd for C₂₁H₂₂N₅O (MH⁺) m/z 360.1819, found 360.1824 (−1.3 ppm). HPLC purity 99.8%.

### Example 60: SN39323 1-Cyclopentyl-6-(isoquinolin-5-ylamino)-3-methyl-1,3-dihydro-2H-imidazo[4,5-c]pyridin-2-one (66)

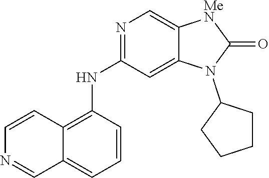 [SMILES: Cn1c(=O)n(C2CCCC2)c2cc(Nc3cccc4cnccc34)ncc21]

A degassed mixture of chloride 6 (120 mg, 0.48 mmol), isoquinolin-5-amine (83 mg, 0.57 mmol), Pd₂dba₃ (22 mg, 24 μmol), XPhos (46 mg, 96 μmol) and Cs₂CO₃ (344 mg, 1.06 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 66 (73 mg, 43%) as a cream powder: mp (EtOAc/MeOH) 234-237° C.; ¹H NMR (CDCl₃) δ 9.28 (d, J=0.6 Hz, 1H, H-1′), 8.53 (d, J=6.0 Hz, 1H, H-3′), 7.86 (s, 1H, H-4), 7.73-7.82 (m, 3H, H-4′, H-6′, H-8′), 7.58 (dd, J=7.9, 7.8 Hz, 1H, H-7), 6.83 (br s, 1H, 6-NH), 6.44 (d, J=0.6 Hz, 1H, H-7), 4.74 (pent, J=8.8 Hz, 1H, 1-CH), 3.41 (s, 3H, 3-CH₃), 1.83-1.99 (m, 4H, 2×CH₂), 1.54-1.67 (m, 4H, 2×CH₂); MS m/z 360.2 (MH⁺, 100%); HRMS calcd for C₂₁H₂₂N₅O (MH⁺) m/z 360.1819, found 360.1822 (−0.9 ppm). HPLC purity 93.6%.

### Example 61: SN39325 1-Cyclopentyl-3-methyl-6-((2-methylquinolin-4-yl)amino)-1,3-dihydro-2H-imidazo[4,5-c]pyridin-2-one (67)

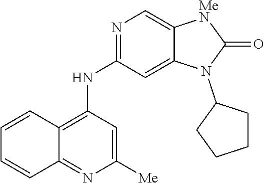 [SMILES: Cc1cc(Nc2cc3c(cn2)n(C)c(=O)n3C2CCCC2)c2ccccc2n1]

A degassed mixture of chloride 6 (128 mg, 0.51 mmol), 2-methylquinolin-4-amine (97 mg, 0.61 mmol), Pd₂dba₃ (23 mg, 26 μmol), XPhos (48 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 67 (134 mg, 71%) as tan crystals: mp (EtOAc/MeOH) 233-236° C.; ¹H NMR (CDCl₃) δ 8.01 (dd, J=8.4, 0.6 Hz, 1H, H-5′), 7.98 (s, 1H, H-3′), 7.94 (dd, J=8.3, 0.6 Hz, 1H, H-8′), 7.68 (ddd, J=8.3, 7.0, 1.2 Hz, 1 H, H-7), 7.44-7.50 (m, 2H, H-4, H-6′), 7.27 (br s, 1H, 6-NH), 6.92 (s, 1H, H-7), 4.87 (pent, J=8.8 Hz, 1H, 1-CH), 3.46 (s, 3H, 3-CH₃), 2.65 (s, 3H, 2′-CH₃), 1.95-2.10 (m, 4H, 2×CH₂), 1.80-1.90 (m, 2H, CH₂), 1.68-1.75 (m, 2H, CH₂); MS m/z 374.2 (MH⁺, 100%); HRMS calcd for C₂₂H₂₄N₅O (MH⁺) m/z 374.1975, found 374.1986 (−2.8 ppm). HPLC purity 100.0%.

### Example 62: SN39326 1-Cyclopentyl-3-methyl-6-(quinoxalin-6-ylamino)-1,3-dihydro-2H-imidazo[4,5-c]pyridin-2-one (68)

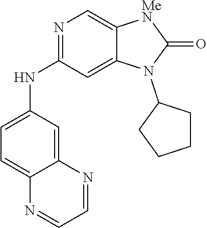 [SMILES: Cn1c(=O)n(C2CCCC2)c2cc(Nc3ccc4nccnc4c3)ncc21]

A degassed mixture of chloride 6 (121 mg, 0.48 mmol), quinoxalin-6-amine (83 mg, 0.58 mmol), Pd₂dba₃ (22 mg, 24 μmol), XPhos (46 mg, 96 μmol) and Cs₂CO₃ (344 mg, 1.06 mmol) in dioxane (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 68 (146 mg, 84%) as a yellow powder: mp (EtOAc/pet. ether) 230-232° C.; ¹H NMR (CDCl₃) δ 8.74 (d, J=1.9 Hz, 1H, H-2′), 8.65 (d, J=1.9 Hz, 1H, H-3′), 8.13 (d, J=2.5 Hz, 1H, H-5′), 8.00 (d, J=9.1 Hz, 1H, H-8′), 7.93 (s, 1H, H-4), 7.67 (dd, J=9.1, 2.5 Hz, 1H, H-7), 6.99 (br s, 1H, 6-NH), 6.77 (s, 1H, H-7), 4.81 (pent, J=8.8 Hz, 1H, 1-CH), 3.44 (s, 3H, 3-CH₃), 2.01-2.10 (m, 4H, 2×CH₂), 1.86-1.96 (m, 2H, CH₂), 1.66-1.76 (m, 2H, CH₂); MS m/z 361.2 (MH⁺, 100%); HRMS calcd for C₂₀H₂₁N₆O (MH⁺) m/z 361.1761, found 361.1778 (−1.8 ppm). HPLC purity 100.0%.

### Example 63: SN39402 6-(Benzo[d][1,3]dioxol-5-ylamino)-1-cyclopentyl-3-methyl-1,3-dihydro-2H-imidazo[4,5-c]pyridin-2-one (69)

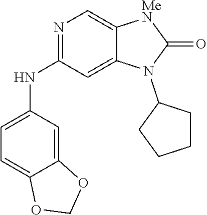 [SMILES: Cn1c(=O)n(C2CCCC2)c2cc(Nc3ccc4c(c3)OCO4)ncc21]

A degassed mixture of chloride 6 (133 mg, 0.53 mmol), benzo[d][1,3]dioxol-5-amine (87 mg, 0.63 mmol), Pd₂dba₃ (24 mg, 24 μmol), XPhos (51 mg, 106 μmol) and Cs₂CO₃ (380 mg, 1.17 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-70% EtOAc/pet. ether) to give imidazopyridinone 69 (130 mg, 70%) as a tan powder: ¹H NMR (CDCl₃) δ 7.78 (s, 1H, H-4), 6.88 (d, J=2.1 Hz, 1H, H-4′), 6.78 (d, J=8.2 Hz, 1H, H-7), 6.69 (dd, J=8.2, 2.2 Hz, 1H, H-6′), 6.45 (d, J=0.4 Hz, 1H, H-7), 6.31 (br s, 1H, 6-NH), 5.96 (s, 2H, H-2′), 4.73 (pent, J=8.8 Hz, 1H, 1-CH), 3.38 (s, 3H, 3-CH₃), 1.95-2.04 (m, 4H, 2×CH₂), 1.80-1.90 (m, 2H, CH₂), 1.62-1.70 (m, 2H, CH₂); MS m/z 353.2 (MH⁺, 100%). HPLC purity 99.8%.

### Example 64: SN39441 6-(Benzo[d]thiazol-6-ylamino)-1-cyclopentyl-3-methyl-1,3-dihydro-2H-imidazo[4,5-c]pyridin-2-one (70)

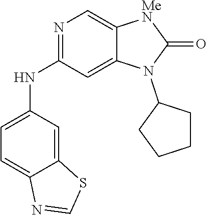 [SMILES: Cn1c(=O)n(C2CCCC2)c2cc(Nc3ccc4ncsc4c3)ncc21]

A degassed mixture of chloride 6 (126 mg, 0.50 mmol), benzo[d]thiazol-6-amine (90 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-100% EtOAc/pet. ether) to give imidazopyridinone 70 (12 mg, 7%) as a gum: ¹H NMR (CDCl₃) δ 8.84 (s, 1H, H-2′), 8.12 (d, J=2.2 Hz, 1H, H-7), 8.03 (d, J=8.8 Hz, 1H, H-4′), 7.87 (s, 1H, H-4), 7.33 (dd, J=8.8, 2.2 Hz, 1H, H-5′), 6.65 (br s, 1H, 6-NH), 6.61 (s, 1H, H-7), 4.79 (pent, J=8.7 Hz, 1H, 1-CH), 3.41 (s, 3H, 3-CH₃), 1.98-2.04 (m, 4H, 2×CH₂), 1.82-1.90 (m, 2H, CH₂), 1.65-1.74 (m, 2H, CH₂); MS m/z 366.2 (MH⁺, 100%); HRMS calcd for C₁₉H₂₀N₅OS (MH⁺) m/z 366.1370, found 366.1374 (−1.2 ppm). HPLC purity 96.3%.

### Example 65: SN39333 1-Cyclopentyl-3-methyl-6-(pyridin-4-ylamino)-1,3-dihydro-2H-imidazo[4,5-c]pyridin-2-one (71)

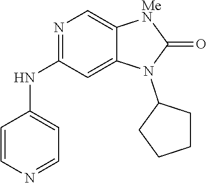 [SMILES: Cn1c(=O)n(C2CCCC2)c2cc(Nc3ccncc3)ncc21]

A degassed mixture of chloride 6 (124 mg, 0.49 mmol), 4-aminopyridine (56 mg, 0.59 mmol), Pd₂dba₃ (22 mg, 25 μmol), XPhos (47 mg, 98 μmol) and Cs₂CO₃ (351 mg, 1.08 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-20%) of MeOH/EtOAc, to give imidazopyridinone 71 (54 mg, 35%) as a white powder: mp (EtOAc) 250-252° C.; ¹H NMR (CDCl₃) δ 8.37 (dd, J=4.9, 1.4 Hz, 2 H, H-2′, H-6′), 7.93 (s, 1H, H-4), 7.28 (dd, J=4.9, 1.5 Hz, 2 H, H-3′, H-5′), 7.00 (br s, 1H, 6-NH), 6.67 (d, J=0.4 Hz, 1 H, H-7), 4.81 (pent, J=8.8 Hz, 1H, 1-CH), 3.43 (s, 3H, 3-CH₃), 1.99-2.09 (m, 4H, 2×CH₂), 1.87-1.97 (m, 2H, CH₂), 1.68-1.78 (m, 2H, CH₂); MS m/z 310.2 (MH⁺, 100%); HRMS calcd for C₁₇H₂₀N₅O (MH⁺) m/z 310.1662, found 310.1668 (−1.8 ppm). HPLC purity 98.0%.

### Example 66: SN39334 1-Cyclopentyl-3-methyl-6-(pyridin-3-ylamino)-1,3-dihydro-2H-imidazo[4,5-c]pyridin-2-one (72)

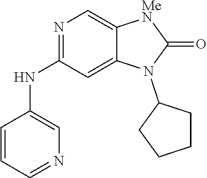 [SMILES: Cn1c(=O)n(C2CCCC2)c2cc(Nc3cccnc3)ncc21]

A degassed mixture of chloride 6 (117 mg, 0.47 mmol), 3-aminopyridine (53 mg, 0.56 mmol), Pd₂dba₃ (22 mg, 25 μmol), XPhos (45 mg, 94 μmol) and Cs₂CO₃ (337 mg, 1.03 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 72 (121 mg, 84%) as a white powder: mp (EtOAc) 203-206° C.; ¹H NMR (CDCl₃) δ 8.57 (d, J=2.5 Hz, 1H, H-2′), 8.23 (dd, J=4.7, 1.4 Hz, 1H, H-6′), 7.88 (ddd, J=8.3, 2.7, 1.4 Hz, 1H, H-5′), 7.84 (s, 1H, H-4), 7.23 (dd, J=8.3, 4.7 Hz, 1H, H-4′), 6.50-6.55 (m, 2H, 6-NH, H-7), 4.79 (pent, J=8.8 Hz, 1H, 1-CH), 3.40 (s, 3H, 3-CH₃), 1.97-2.07 (m, 4H, 2×CH₂), 1.83-1.95 (m, 2H, CH₂), 1.65-1.68 (m, 2H, CH₂); MS m/z 310.2 (MH⁺, 100%); HRMS calcd for C₁₇H₂₀N₅O (MH⁺) m/z 310.1662, found 310.1668 (−1.9 ppm). HPLC purity 98.5%.

### Example 67: SN39341 1-Cyclopentyl-3-methyl-6-(pyridin-2-ylamino)-1,3-dihydro-2H-imidazo[4,5-c]pyridin-2-one (73)

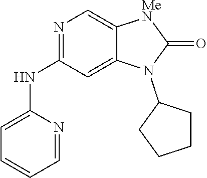 [SMILES: Cn1c(=O)n(C2CCCC2)c2cc(Nc3ccccn3)ncc21]

A degassed mixture of chloride 6 (122 mg, 0.49 mmol), 2-aminopyridine (55 mg, 0.58 mmol), Pd₂dba₃ (22 mg, 25 μmol), XPhos (47 mg, 98 μmol) and Cs₂CO₃ (351 mg, 1.08 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-5%) of MeOH/EtOAc, to give imidazopyridinone 73 (112 mg, 75%) as a tan powder: mp (EtOAc) 151-153° C.; ¹H NMR (CDCl₃) δ 8.25 (ddd, J=5.0, 1.8, 0.7 Hz, 1H, H-6′), 7.93 (s, 1H, H-4), 7.86 (s, 1H, H-7), 7.56 (ddd, J=8.4, 7.2, 1.9 Hz, 1H, H-4′), 7.49 (br s, 1H, 6-NH), 7.10 (d, J=8.4 Hz, 1H, H-3′), 6.81 (ddd, J=7.2, 5.0, 0.8 Hz, 1H, H-5′), 4.87 (pent, J=8.4 Hz, 1H, 1-CH), 3.41 (s, 3H, 3-CH₃), 1.98-2.13 (m, 6H, 3×CH₂), 1.70-1.80 (m, 2H, CH₂); MS m/z 310.2 (MH⁺, 100%); HRMS calcd for C₁₇H₂₀N₅O (MH⁺) m/z 310.1662, found 310.1665 (−0.8 ppm). HPLC purity 99.9%.

### Example 68: SN39344 1-Cyclopentyl-3-methyl-6-((3-methylpyridin-4-yl)amino)-1,3-dihydro-2H-imidazo[4,5-c]pyridin-2-one (74)

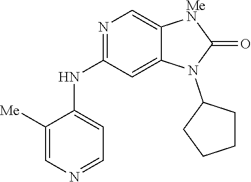 [SMILES: Cc1cnccc1Nc1cc2c(cn1)n(C)c(=O)n2C1CCCC1]

A degassed mixture of chloride 6 (137 mg, 0.54 mmol), 3-methylpyridin-4-amine (71 mg, 0.65 mmol), Pd₂dba₃ (25 mg, 27 μmol), XPhos (51 mg, 108 μmol) and Cs₂CO₃ (387 mg, 1.19 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-20%) of MeOH/EtOAc, to give imidazopyridinone 74 (150 mg, 85%) as brown cubes: mp (EtOAc) 217-220° C.; ¹H NMR (CDCl₃) δ 8.27-8.31 (m, 2H, H-2′, H-6′), 7.93 (s, 1H, H-4), 7.52 (d, J=5.7 Hz, 1H, H-5′), 6.76 (d, J=0.4 Hz, 1H, H-7), 6.40 (br s, 1H, 6-NH), 4.81 (pent, J=8.8 Hz, 1H, 1-CH), 3.44 (s, 3H, 3-CH₃), 2.74 (s, 3H, 3′-CH₃), 2.00-2.09 (m, 4H, 2×CH₂), 1.78-1.85 (m, 2H, CH₂), 1.70-1.78 (m, 2H, CH₂); MS m/z 324.2 (MH⁺, 100%); HRMS calcd for C₁₈H₂₂N₅O (MH⁺) m/z 324.1819, found 324.1825 (−1.9 ppm). HPLC purity 99.5%.

### Example 69: SN39361 1-Cyclopentyl-3-methyl-6-((2-methylpyridin-4-yl)amino)-1,3-dihydro-2H-imidazo[4,5-c]pyridin-2-one (75)

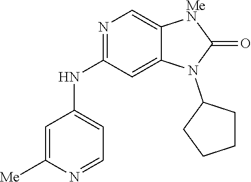 [SMILES: Cc1cc(Nc2cc3c(cn2)n(C)c(=O)n3C2CCCC2)ccn1]

A degassed mixture of chloride 6 (129 mg, 0.51 mmol), 2-methylpyridin-4-amine (67 mg, 0.61 mmol), Pd₂dba₃ (23 mg, 25 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 75 (127 mg, 77%) as a cream powder: mp (EtOAc) 214-216° C.; ¹H NMR (CDCl₃) δ 8.25 (d, J=5.8 Hz, 1H, H-6′), 7.93 (s, 1H, H-4), 7.16 (d, J=2.1 Hz, 1H, H-3′), 7.06 (dd, J=5.8, 2.2 Hz, 1H, H-5′), 7.03 (br s, 1H, 6-NH), 6.70 (s, 1H, H-7), 4.83 (pent, J=8.7 Hz, 1H, 1-CH), 3.43 (s, 3H, 3-CH₃), 2.49 (s, 3H, 2′-CH₃), 2.00-2.10 (m, 4H, 2×CH₂), 1.87-1.96 (m, 2H, CH₂), 1.70-1.78 (m, 2H, CH₂); MS m/z 324.2 (MH⁺, 100%); HRMS calcd for C₁₈H₂₂N₅O (MH⁺) m/z 324.1819, found 324.1820 (−0.2 ppm). HPLC purity 99.0%.

### Example 70: SN39346 1-Cyclopentyl-3-methyl-6-((2-methylpyridin-3-yl)amino)-1,3-dihydro-2H-imidazo[4,5-c]pyridin-2-one (76)

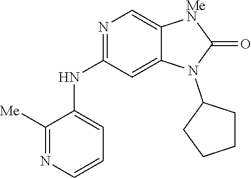 [SMILES: Cc1ncccc1Nc1cc2c(cn1)n(C)c(=O)n2C1CCCC1]

A degassed mixture of chloride 6 (125 mg, 0.50 mmol), 2-methylpyridin-3-amine (64 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 76 (103 mg, 64%) as tan crystals: mp (EtOAc) 144-146° C.; ¹H NMR (CDCl₃) δ 8.22 (dd, J=4.8, 1.4 Hz, 1H, H-6′), 7.82-7.86 (m, 2H, H-4, H-4′), 7.14 (dd, J=8.1, 4.8 Hz, 1H, H-5′), 6.41 (d, J=0.6 Hz, 1H, H-7), 6.14 (br s, 1H, 6-NH), 4.75 (pent, J=8.8 Hz, 1H, 1-CH), 3.41 (s, 3H, 3-CH₃), 2.55 (s, 3H, 2′-CH₃), 1.95-2.05 (m, 4H, 2×CH₂), 1.80-1.88 (m, 2H, CH₂), 1.64-1.72 (m, 2H, CH₂); MS m/z 324.2 (MH⁺, 100%); HRMS calcd for C₁₈H₂₂N₅O (MH⁺) m/z 324.1819, found 324.1819 (−0.1 ppm). HPLC purity 99.7%.

### Example 71: SN39362 1-Cyclopentyl-3-methyl-6-((5-methylpyridin-3-yl)amino)-1,3-dihydro-2H-imidazo[4,5-c]pyridin-2-one (77)

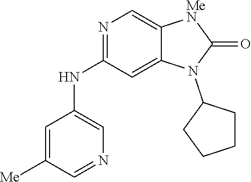 [SMILES: Cc1cncc(Nc2cc3c(cn2)n(C)c(=O)n3C2CCCC2)c1]

A degassed mixture of chloride 6 (118 mg, 0.47 mmol), 5-methylpyridin-3-amine (61 mg, 0.56 mmol), Pd₂dba₃ (22 mg, 24 μmol), XPhos (45 mg, 94 μmol) and Cs₂CO₃ (337 mg, 1.03 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 77 (129 mg, 78%) as a cream powder: mp (EtOAc) 182-185° C.; ¹H NMR (CDCl₃) δ 8.37 (d, J=2.5 Hz, 1H, H-6′), 8.07 (d, J=1.2 Hz, 1H, H-2′), 7.85 (s, 1H, H-4), 7.69 (br s, 1H, H-4′), 6.62 (br s, 1H, 6-NH), 6.55 (d, J=0.5 Hz, 1H, H-7), 4.79 (pent, J=8.8 Hz, 1H, 1-CH), 3.40 (s, 3H, 3-CH₃), 2.33 (s, 3H, 5′-CH₃), 1.96-2.07 (m, 4H, 2×CH₂), 1.84-1.92 (m, 2H, CH₂), 1.68-1.75 (m, 2H, CH₂); MS m/z 324.2 (MH⁺, 100%); HRMS calcd for C₁₈H₂₂N₅O (MH⁺) m/z 324.1819, found 324.1821 (−0.6 ppm). HPLC purity 94.3%.

### Example 72: SN39342 1-Cyclopentyl-3-methyl-6-((4-methylpyridin-3-yl)amino)-1,3-dihydro-2H-imidazo[4,5-c]pyridin-2-one (78)

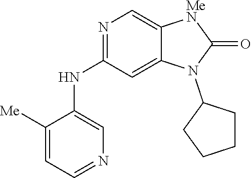 [SMILES: Cc1ccncc1Nc1cc2c(cn1)n(C)c(=O)n2C1CCCC1]

A degassed mixture of chloride 6 (127 mg, 0.51 mmol), 4-methylpyridin-3-amine (66 mg, 0.61 mmol), Pd₂dba₃ (23 mg, 26 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 78 (99 mg, 61%) as a brown foam: ¹H NMR (CDCl₃) δ 8.69 (s, 1H, H-2′), 8.25 (d, J=4.8 Hz, 1H, H-6′), 7.81 (s, 1H, H-4), 7.17 (d, J=4.8 Hz, 1H, H-5′), 6.39 (d, J=0.5 Hz, 1H, H-7), 6.14 (br s, 1H, 6-NH), 4.70 (pent, J=8.8 Hz, 1H, 1-CH), 3.40 (s, 3H, 3-CH₃), 2.30 (s, 3H, 4′-CH₃), 1.95-2.02 (m, 4H, 2×CH₂), 1.80-1.90 (m, 2H, CH₂), 1.63-1.72 (m, 2H, CH₂); MS m/z 324.2 (MH⁺, 100%); HRMS calcd for C₁₈H₂₂N₅O (MH⁺) m/z 324.1819, found 324.1816 (1.0 ppm). HPLC purity 98.6%.

### Example 73: SN39360 1-Cyclopentyl-3-methyl-6-((3-methylpyridin-2-yl)amino)-1,3-dihydro-2H-imidazo[4,5-c]pyridin-2-one (79)

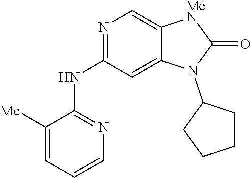 [SMILES: Cc1cccnc1Nc1cc2c(cn1)n(C)c(=O)n2C1CCCC1]

A degassed mixture of chloride 6 (125 mg, 0.50 mmol), 3-methylpyridin-2-amine (64 mg, 0.60 mmol), Pd₂dba₃ (23 mg, 25 μmol), XPhos (48 mg, 100 μmol) and Cs₂CO₃ (358 mg, 1.10 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (70-100% EA/pet.ether) to give imidazopyridinone 79 (149 mg, 93%) as a tan powder: mp (EtOAc/pet ether) 169-172° C.; ¹H NMR (CDCl₃) δ 8.04 (d, J=0.5 Hz, 1H, H-4), 7.85 (s, 1H, H-7), 7.45 (dd, J=8.2, 7.4 Hz, 1H, H-5′), 7.28 (s, 1H, 6-NH), 6.79 (d, J=8.2 Hz, 1H, H-6′), 6.67 (d, J=7.4 Hz, 1H, H-4′), 4.92 (pent, J=8.7 Hz, 1H, 1-CH), 3.41 (s, 3H, 3-CH₃), 2.48 (s, 3H, 3′-CH₃), 1.98-2.16 (m, 6H, 3×CH₂), 1.72-1.80 (m, 2H, CH₂); MS m/z 324.2 (MH⁺, 100%); HRMS calcd for C₁₈H₂₂N₅O (MH⁺) m/z 324.1819, found 324.1818 (0.4 ppm). HPLC purity 99.6%.

### Example 74: SN39405 1-Cyclopentyl-6-((6-methoxy-4-methylpyridin-3-yl)amino)-3-methyl-1,3-dihydro-2H-imidazo[4,5-c]pyridin-2-one (80)

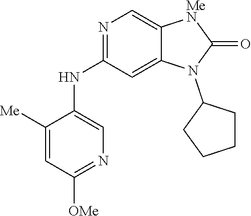 [SMILES: COc1cc(C)c(Nc2cc3c(cn2)n(C)c(=O)n3C2CCCC2)cn1]

A degassed mixture of chloride 6 (129 mg, 0.51 mmol), 5-methoxy-3-methylpyridin-2-amine (85 mg, 0.61 mmol), Pd₂dba₃ (23 mg, 26 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (70-100% EtOAc/pet. ether) to give imidazopyridinone 80 (4 mg, 2%) as a clear oil: ¹H NMR (CDCl₃) δ 8.11 (s, 1H, H-2′), 7.45 (s, 1H, H-4), 6.69 (s, 1H, H-5′), 6.03 (s, 1H, H-7), 5.97 (br s, 1H, 6-NH), 4.64 (pent, J=8.7 Hz, 1H, 1-CH), 3.93 (s, 3H, 6′-OCH₃), 3.37 (s, 3H, 3-CH₃), 2.16 (s, 3H, 4′-CH₃), 1.74-1.82 (m, 4H, 2×CH₂), 1.57-1.67 (m, 2H, CH₂), 1.58-1.67 (m, 2H, CH₂); MS m/z 354.2 (MH⁺, 100%); HRMS calcd for C₁₉H₂₄N₅O₂ (MH⁺) m/z 354.1925, found 354.1922 (0.6 ppm).

### Example 75: SN39395 1-Cyclopentyl-6-((6-methoxypyrimidin-4-yl)amino)-3-methyl-1,3-dihydro-2H-imidazo[4,5-c]pyridin-2-one (81)

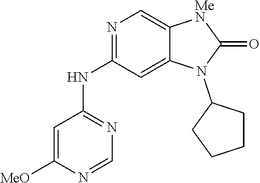 [SMILES: COc1cc(Nc2cc3c(cn2)n(C)c(=O)n3C2CCCC2)ncn1]

A degassed mixture of chloride 6 (128 mg, 0.51 mmol), 6-methoxypyrimidin-4-amine (76 mg, 0.61 mmol), Pd₂dba₃ (23 mg, 25 μmol), XPhos (49 mg, 102 μmol) and Cs₂CO₃ (366 mg, 1.12 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 81 (88 mg, 51%) as white crystals: mp (EtOAc/pet ether) 180-182° C.; ¹H NMR (CDCl₃) δ 8.44 (d, J=0.8 Hz, 1H, H-2′), 7.90 (d, J=0.4 Hz, 1H, H-4), 7.82 (br s, 1H, 6-NH), 7.43 (s, 1H, H-5′), 6.76 (d, J=0.7 Hz, 1H, H-7), 4.85 (pent, J=8.8 Hz, 1H, 1-CH), 3.96 (s, 3H, 6′-OCH₃), 3.43 (s, 3H, 3-CH₃), 1.94-2.10 (m, 6H, 3×CH₂), 1.72-1.80 (m, 2H, CH₂); HRMS calcd for C₁₇H₂₁N₆O₂ (MH⁺) m/z 341.1721, found 371.1711 (2.7 ppm). HPLC purity 99.8%.

### Example 76: SN39258 6-((4-Aminophenyl)amino)-1-cyclopentyl-3-methyl-1,3-dihydro-2H-imidazo[4,5-c]pyridin-2-one (82)

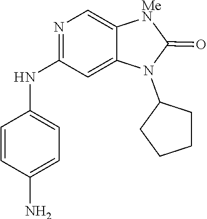 [SMILES: Cn1c(=O)n(C2CCCC2)c2cc(Nc3ccc(N)cc3)ncc21]

A mixture of nitroaniline 17 (66 mg, 0.19 mmol) and Pd/C (10 mg) in EtOH/EtOAc (1:1, 30 mL) was stirred under Hz (50 psi) for 3 h. The mixture was filtered through diatomaceous earth, the pad was washed with EtOH (20 mL) and the combined filtrate evaporated. The residue was crystallized to give imidazopyridinone 82 (40 mg, 66%) as a brown powder: mp (EtOAc/pet ether) 171-173° C.; ¹H NMR (CDCl₃) δ 7.74 (d, J=0.4 Hz, 1H, H-4), 7.08 (ddd, J=8.6, 3.1, 2.1 Hz, 2 H, H-2′, H-6′), 6.71 (ddd, J=8.6, 3.1, 2.1 Hz, 2 H, H-3′, H-5′), 6.37 (d, J=0.6 Hz, 1H, H-7), 6.21 (s, 1H, 6-NH), 4.70 (pent, J=8.7 Hz, 1H, 1-CH), 3.62 (br s, 2H, 4′-NH₂), 3.37 (s, 3H, 3-CH₃), 1.92-1.98 (m, 4H, 2×CH₂), 1.76-1.86 (m, 2H, CH₂), 1.59-1.60 (m, 2H, CH₂); MS m/z 324.2 (MH⁺, 100%). Anal calcd for C₁₈H₂₁N₅O·¼H₂O: C, 65.93; H, 6.61; N, 21.36. Found: C, 65.90; H, 6.37; N, 21.47%. HPLC purity 99.3%.

### Example 77: SN39259 6-((3-Aminophenyl)amino)-1-cyclopentyl-3-methyl-1,3-dihydro-2H-imidazo[4,5-c]pyridin-2-one (83)

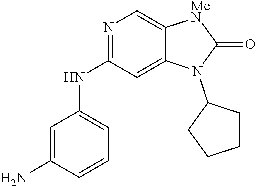 [SMILES: Cn1c(=O)n(C2CCCC2)c2cc(Nc3cccc(N)c3)ncc21]

A mixture of nitroaniline 18 (80 mg, 0.23 mmol) and Pd/C (10 mg) in EtOH/EtOAc (1:1, 30 mL) was stirred under H2 (50 psi) for 3 h. The mixture was filtered through diatomaceous earth, the pad was washed with EtOH (20 mL) and the combined filtrate evaporated. The residue was crystallized to give imidazopyridinone 83 (40 mg, 54%) as a tan powder: mp (EtOAc/pet ether) 151-153° C.; ¹H NMR (CDCl₃) δ 7.81 (s, 1H, H-4), 7.10 (d, J=7.9 Hz, 1H, H-5′), 6.67 (d, J=0.6 Hz, 1H, H-7), 6.61-6.65 (m, 2H, H-2′, H-6′), 6.39 (br s, 1H, 6-NH), 7.35 (ddd, J=7.9, 2.1, 0.9 Hz, 1H, H-4′), 4.77 (pent, J=8.7 Hz, 1H, 1-CH), 3.68 (br s, 2H, 3′-NH₂), 3.39 (s, 3H, 3-CH₃), 1.97-2.05 (m, 4H, 2×CH₂), 1.83-1.93 (m, 2H, CH₂), 1.64-1.74 (m, 2H, CH₂); MS m/z 324.2 (MH⁺, 100%). Anal calcd for C₁₈H₂₁N₅O: C, 66.85; H, 6.55; N, 21.66. Found: C, 66.93; H, 6.56; N, 21.57%. HPLC purity 97.9%.

### Example 78: SN39272 6-((2-Aminophenyl)amino)-1-cyclopentyl-3-methyl-1,3-dihydro-2H-imidazo[4,5-c]pyridin-2-one (84)

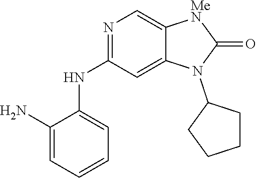 [SMILES: Cn1c(=O)n(C2CCCC2)c2cc(Nc3ccccc3N)ncc21]

A mixture of nitroaniline 19 (100 mg, 0.28 mmol) and Pd/C (10 mg) in EtOH/EtOAc (1:1, 30 mL) was stirred under H₂ (50 psi) for 3 h. The mixture was filtered through diatomaceous earth, the pad was washed with EtOH (20 mL) and the combined filtrate evaporated. The residue was crystallized to give imidazopyridinone 84 (56 mg, 62%) as pink needles: mp (EtOAc/pet ether) 150-151° C.; ¹H NMR (CDCl₃) δ 7.77 (s, 1H, H-4), 7.18 (dd, J=7.8, 1.3 Hz, 1 H, H-6′), 7.07 (ddd, J=7.7, 7.6, 1.4 Hz, 1H, H-4′), 6.83 (dd, J=7.9, 1.4 Hz, 1H, H-3′), 6.78 (dt, J=7.6, 1.4 Hz, 1H, H-5′), 6.14 (d, J=0.6 Hz, 1H, H-7), 6.03 (br s, 1H, 6-NH), 4.68 (pent, J=8.7 Hz, 1H, 1-CH), 3.86 (br s, 2H, 2′-NH₂), 3.39 (s, 3H, 3-CH₃), 1.89-1.97 (m, 4H, 2×CH₂), 1.70-1.80 (m, 2H, CH₂), 1.56-1.66 (m, 2H, CH₂); MS m/z 324.2 (MH⁺, 100%). Anal calcd for C₁₈H₂₁N₅O: C, 66.85; H, 6.55; N, 21.66. Found: C, 66.97; H, 6.57; N, 21.44%. HPLC purity 99.6%.

### Example 79: SN39305 1-Cyclopentyl-6-((4-hydroxyphenyl)amino)-3-methyl-1,3-dihydro-2H-imidazo[4,5-c]pyridin-2-one (85)

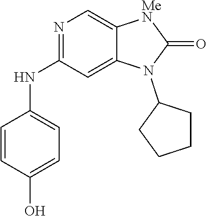 [SMILES: Cn1c(=O)n(C2CCCC2)c2cc(Nc3ccc(O)cc3)ncc21]

A mixture of benzyl ether 34 (130 mg, 0.31 mmol) and Pd/C (25 mg) in a mixture of EtOAc (25 mL) and EtOH (25 mL) was stirred under Hz (50 psi) at 20° C. for 16 h. The mixture was filtered through diatomaceous earth and the filtrate was evaporated. The residue was purified by chromatography, eluting with a gradient (80-100%) of EtOAc/pet. ether, to give imidazopyridinone 85 (73 mg, 72%) as cream crystals: mp (EtOAc/pet ether) 257-260° C.; 1H NMR [(CD₃)₂SO] δ 8.84 (s, 1H, 4′-OH), 8.34 (s, 1H, 6-NH), 7.83 (s, 1H, H-4), 7.36 (ddd, J=8.9, 3.4, 2.1 Hz, 2 H, H-2′, H-6′), 6.65 (ddd, J=8.9, 3.4, 2.1 Hz, 2 H, H-3′, H-5′), 6.50 (s, 1H, H-7), 4.70 (pent, J=8.4 Hz, 1H, 1-CH), 3.27 (s, 3H, 3-CH₃), 1.85-1.96 (m, 6H, 3×CH₂), 1.63-1.70 (m, 2H, CH₂); MS m/z 325.2 (MH⁺, 100%). Anal. calcd for C₁₈H₂₀N₄O₂·0.2EtOAc: C, 66.02; H, 6.38; N, 16.38. Found: C, 66.10; H, 6.61; N, 16.73%. HPLC purity 99.9%.

### Example 80: SN39306 1-Cyclopentyl-6-((3-hydroxyphenyl)amino)-3-methyl-1,3-dihydro-2H-imidazo[4,5-c]pyridin-2-one (86)

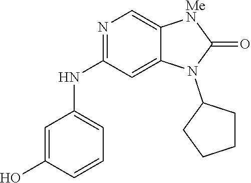 [SMILES: Cn1c(=O)n(C2CCCC2)c2cc(Nc3cccc(O)c3)ncc21]

A mixture of benzyl ether 35 (198 mg, 0.31 mmol) and Pd/C (25 mg) in a mixture of EtOAc (25 mL) and EtOH (25 mL) was stirred under Hz (50 psi) at 20° C. for 16 h. The mixture was filtered through diatomaceous earth and the filtrate was evaporated. The residue was purified by chromatography, eluting with a gradient (80-100%) of EtOAc/pet. ether, to give imidazopyridinone 86 (97 mg, 63%) as white crystals: mp (EtOAc/pet ether) 216-218° C.; 1H NMR [(CD₃)₂SO] δ 9.11 (s, 1H, 4′-OH), 8.64 (s, 1H, 6-NH), 7.92 (s, 1H, H-4), 7.20 (d, J=2.0 Hz, 1H, H-2′), 7.93-7.01 (m, 2H, H-5′, H-6′), 6.64 (s, 1H, H-7), 6.24 (dt, J=6.9, 2.2 Hz, 1 H, H-4′), 4.72 (pent, J=8.8 Hz, 1H, 1-CH), 3.30 (s, 3H, 3-CH₃), 1.87-1.97 (m, 6H, 3×CH₂), 1.65-1.72 (m, 2H, CH₂); MS m/z 325.2 (MH⁺, 100%). Anal. calcd for C₁₈H₂₀N₄O₂·0.2EtOAc: C, 66.02; H, 6.37; N, 16.38. Found: C, 65.91; H, 6.50; N, 16.38%. HPLC purity 99.2%.

### Example 81: SN39329 1-Cyclopentyl-6-((2-hydroxyphenyl)amino)-3-methyl-1,3-dihydro-2H-imidazo[4,5-c]pyridin-2-one (87)

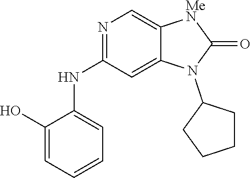 [SMILES: Cn1c(=O)n(C2CCCC2)c2cc(Nc3ccccc3O)ncc21]

A mixture of benzyl ether 36 (108 mg, 0.26 mmol) and Pd/C (20 mg) in a mixture of EtOAc (25 mL) and EtOH (25 mL) was stirred under H2 (50 psi) at 20° C. for 16 h. The mixture was filtered through diatomaceous earth and the filtrate was evaporated. The residue was purified by chromatography, eluting with a gradient (50-100%) of EtOAc/pet. ether, to give imidazopyridinone 87 (46 mg, 55%) as a white powder: mp (EtOAc/pet. ether) 160-163° C.; 1H NMR [(CD₃)₂SO] δ 10.53 (s, 1H, OH), 8.14 (br s, 1H, 6-NH), 7.88 (s, 1H, H-4), 7.78 (dd, J=7.7, 1.8 Hz, 1H, H-6′), 6.88 (s, 1H, H-7), 6.82 (dd, J=7.7, 1.8 Hz, 1H, H-3′), 6.77 (ddd, J=7.7, 7.2, 1.8 Hz, 1H, H-4′), 6.72 (ddd, J=7.7, 7.2, 1.8 Hz, 1H, H-5′), 4.70 (pent, J=8.6 Hz, 1 H, 1-CH), 3.29 (s, 3H, 3-CH₃), 1.89-1.99 (m, 6H, 3×CH₂), 1.60-1.70 (m, 2H, CH₂); MS m/z 325.2 (MH⁺, 100%); HRMS calcd for C₁₈H₂₁N₄O₂ (MH⁺) m/z 325.1659, found 325.1666 (−2.2 ppm). HPLC purity 99.0%.

### Example 82: SN39375 1-Cyclopentyl-6-((4-hydroxy-2-methylphenyl)amino)-3-methyl-1,3-dihydro-2H-imidazo[4,5-c]pyridin-2-one (88)

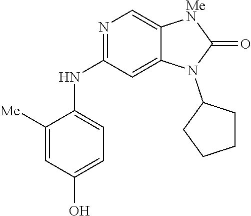 [SMILES: Cc1cc(O)ccc1Nc1cc2c(cn1)n(C)c(=O)n2C1CCCC1]

A mixture of benzyl ether 51 (172 mg, 0.40 mmol) and Pd/C (20 mg) in a mixture of EtOAc (25 mL) and EtOH (25 mL) was stirred under Hz (50 psi) at 20° C. for 16 h. The mixture was filtered through diatomaceous earth and the filtrate was evaporated. The residue was purified by chromatography, eluting with a gradient (50-100%) of EtOAc/pet. ether, to give imidazopyridinone 88 (44 mg, 32%) as a pink powder: mp (EtOAc/pet. ether) 244-246° C.; 1H NMR [(CD₃)₂SO] δ 9.03 (s, 1H, 4′-OH), 7.76 (br s, 1H, 6-NH), 7.57 (s, 1H, H-4), 7.14 (d, J=8.5 Hz, 1H, H-6′), 6.54 (dd, J=8.5, 2.8 Hz, 1H, H-5′), 6.62 (d, J=2.7 Hz, 1H, H-3), 6.25 (s, 1H, H-7), 4.64 (pent, J=8.7 Hz, 1H, 1-CH), 3.25 (s, 3H, 3-CH₃), 2.09 (s, 3H, 2′-CH₃), 1.82-1.89 (m, 4H, 2×CH₂), 1.72-1.82 (m, 2H, CH₂), 1.58-1.68 (m, 2H, CH₂); MS m/z 339.2 (MH⁺, 100%); HRMS calcd for C₁₉H₂₃N₄O₂ (MH⁺) m/z 339.1816, found 339.1811 (1.3 ppm). HPLC purity 99.2%.

### Example 83: SN39777 3-Benzyl-1-cyclopentyl-6-((4-methoxy-2-methylphenyl)amino)-1,3-dihydro-2H-imidazo[4,5-c]pyridin-2-one (94)

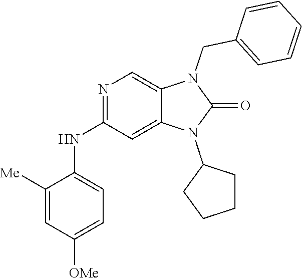 [SMILES: COc1ccc(Nc2cc3c(cn2)n(Cc2ccccc2)c(=O)n3C2CCCC2)c(C)c1]

3-Benzyl-6-chloro-1-cyclopentyl-1,3-dihydro-2H-imidazo[4,5-c]pyridin-2-one (89). NaH (60% dispersion, 65 mg, 1.62 mmol) was added to a stirred solution of imidazopyridinone 5 (321 mg, 1.35 mmol) and benzyl bromide (0.24 mL, 2.03 mmol) in dry DMF (5 mL) at 5° C. The mixture was stirred at 20° C. for 16 h and then quenched with ice/water (5 mL) and the mixture was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (10-20%) of EtOAc/pet. ether, to give chloride 89 (352 mg, 80%) as a tan oil: ¹H NMR (CDCl₃) δ 7.83 (d, J=0.5 Hz, 1H, H-4), 7.27-7.38 (m, 5H, aryl-H), 6.98 (d, J=0.5 Hz, 1H, H-7), 5.05 (s, 2H, 3-CH₂), 4.82 (pent, J=8.7 Hz, 1H, 1-CH), 1.93-2.10 (m, 6H, 3×CH₂), 1.70-1.80 (m, 2H, CH₂); MS m/z 328.2 (MH⁺, 100%), 330.2 (MH⁺, 35%). HRMS calcd for C₁₈H₁₉₃₅ClN₃O (MH⁺) m/z 328.1211, found 328.1223 (−3.7 ppm).

3-Benzyl-1-cyclopentyl-6-((4-methoxy-2-methylphenyl)amino)-1,3-dihydro-2H-imidazo[4,5-c]pyridin-2-one (94). A degassed mixture of chloride 89 (170 mg, 0.52 mmol), 4-methoxy-2-methylaniline (85 mg, 0.62 mmol), Pd₂dba₃ (24 mg, 26 μmol), XPhos (50 mg, 104 μmol) and Cs₂CO₃ (373 mg, 1.14 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with a gradient (40-50%) of EtOAc/pet. ether, to give imidazopyridinone 94 (70 mg, 31%) as a tan foam: ¹H NMR (CDCl₃) δ 7.62 (d, J=0.4 Hz, 1H, H-4), 7.27-7.34 (m, 5H, aryl-H), 7.20 (d, J=8.6 Hz, 1H, H-6′), 6.82 (d, J=2.9 Hz, 1H, H-3′), 6.75 (dd, J=8.6, 2.9 Hz, 1H, H-5′), 6.10 (d, J=0.5 Hz, 1H, H-7), 5.94 (br s, 1H, 6-NH), 4.98 (s, 2H, 3-CH₂), 4.70 (pent, J=8.7 Hz, 1H, 1-CH), 3.81 (s, 3H, 4′-OCH₃), 2.23 (s, 3H, 2′-CH₃), 1.92-1.98 (m, 4H, 2×CH₂), 1.70-1.80 (m, 2H, CH₂), 1.56-1.66 (m, 2H, CH₂); MS m/z 429.2 (MH⁺, 100%). HRMS calcd for C₂₆H₂₉N₄O₂ (MH⁺) m/z 429.2285, found 429.2298 (−3.0 ppm). HPLC purity 98.7%.

### Example 84: SN39778 1,3-Dicyclopentyl-6-((4-methoxy-2-methylphenyl)amino)-1,3-dihydro-2H-imidazo[4,5-c]pyridin-2-one (95)

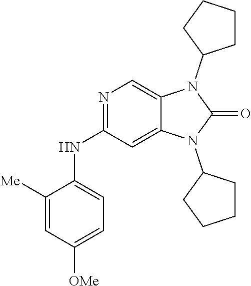 [SMILES: COc1ccc(Nc2cc3c(cn2)n(C2CCCC2)c(=O)n3C2CCCC2)c(C)c1]

6-Chloro-1,3-dicyclopentyl-1,3-dihydro-2H-imidazo[4,5-c]pyridin-2-one (90). NaH (60% dispersion, 61 mg, 1.51 mmol) was added to a stirred solution of pyridinone 5 (300 mg, 1.26 mmol) and iodocyclopentane (0.22 mL, 1.89 mmol) in dry DMF (5 mL) at 5° C. The mixture was stirred at 20° C. for 16 h and then quenched with ice/water (5 mL) and the mixture was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (10-20%) of EtOAc/pet. ether, to give chloride 90 (215 mg, 56%) as a tan oil: ¹H NMR (CDCl₃) δ 8.04 (d, J=0.4 Hz, 1H, H-4), 6.98 (d, J=0.4 Hz, 1H, H-7), 4.75-4.87 (m, 2H, 1-CH, 3-CH), 1.91-2.08 (m, 12H, 6×CH₂), 1.70-1.78 (m, 4H, 2×CH₂); MS m/z 306.2 (MH⁺, 100%), 308.2 (MH⁺, 35%). HRMS calcd for C₁₆H₂₁³⁵ClN₃O (MH⁺) m/z 306.1368, found 306.1374 (−2.2 ppm).

1,3-Dicyclopentyl-6-((4-methoxy-2-methylphenyl)amino)-1,3-dihydro-2H-imidazo[4,5-c]pyridin-2-one (95). A degassed mixture of chloride 90 (190 mg, 0.62 mmol), 4-methoxy-2-methylaniline (103 mg, 0.75 mmol), Pd₂dba₃ (28 mg, 31 μmol), XPhos (59 mg, 124 μmol) and Cs₂CO₃ (444 mg, 1.36 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with a gradient (25-30%) of EtOAc/pet. ether, to give imidazopyridinone 95 (51 mg, 20%) as a tan powder: mp 163-165° C.; ¹H NMR (CDCl₃) δ 7.84 (d, J=0.5 Hz, 1H, H-4), 7.23 (d, J=8.6 Hz, 1H, H-6′), 6.84 (d, J=2.9 Hz, 1H, H-3′), 6.77 (dd, J=8.6, 2.9 Hz, 1H, H-5′), 6.12 (d, J=0.5 Hz, 1H, H-7), 5.96 (br s, 1H, 6-NH), 4.81 (pent, J=8.8 Hz, 1H, 3-CH), 4.68 (pent, J=8.8 Hz, 1H, 1-CH), 3.82 (s, 3H, 4′-OCH₃), 2.25 (s, 3H, 2′-CH₃), 2.00-2.07 (m, 4H, 2×CH₂), 1.88-1.97 (m, 6H, 3×CH₂), 1.69-1.78 (m, 4H, 2×CH₂), 1.57-1.67 (m, 2H, CH₂); MS m/z 407.2 (MH⁺, 100%). HRMS calcd for C₂₄H₃₁N₄O₂ (MH⁺) m/z 407.2441, found 407.2437 (−1.2 ppm). HPLC purity 99.4%.

### Example 85: SN39790 1-Cyclopentyl-3-isopropyl-6-((4-methoxy-2-methylphenyl)amino)-1,3-dihydro-2H-imidazo[4,5-c]pyridin-2-one (96)

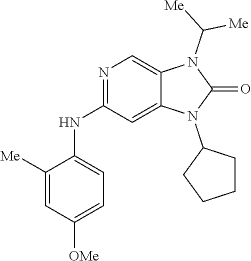 [SMILES: COc1ccc(Nc2cc3c(cn2)n(C(C)C)c(=O)n3C2CCCC2)c(C)c1]

6-Chloro-1-cyclopentyl-3-isopropyl-1,3-dihydro-2H-imidazo[4,5-c]pyridin-2-one (91). NaH (60% dispersion, 61 mg, 1.5 mmol) was added to a stirred solution of pyridinone 5 (0.30 g, 1.3 mmol) and isopropyl bromide (0.18 mL, 1.9 mmol) in dry DMF (5 mL) at 5° C. The mixture was stirred at 20° C. for 16 h and then quenched with ice/water (5 mL) and the mixture was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with 20% EtOAc/pet. ether, to give chloride 91 (235 mg, 67%) as a white solid: mp 129-131° C.; ¹H NMR (CDCl₃) δ 8.12 (d, J=0.4 Hz, 1H, H-4), 6.98 (d, J=0.4 Hz, 1H, H-7), 4.78 (pent, J=8.7 Hz, 1H, 1-CH), 4.72 (sept, J=7.0 Hz, 1H, 3-CH), 1.93-2.07 (m, 6H, 3×CH₂), 1.72-1.78 (m, 2H, CH₂), 1.52 (d, J=7.0 Hz, 6 H, 2×CH₃); MS m/z 280.2 (MH⁺, 100%), 282.2 (MH⁺, 35%). HRMS calcd for C₁₄H₁₉₃₅ClN₃O (MH⁺) m/z 280.1211, found 280.1217 (−2.1 ppm).

1-Cyclopentyl-3-isopropyl-6-((4-methoxy-2-methylphenyl)amino)-1,3-dihydro-2H-imidazo[4,5-c]pyridin-2-one (96). A degassed mixture of chloride 91 (123 mg, 0.44 mmol), 4-methoxy-2-methylaniline (72 mg, 0.53 mmol), Pd₂dba₃ (20 mg, 22 μmol), XPhos (42 mg, 88 μmol) and Cs₂CO₃ (315 mg, 0.97 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with 50% EtOAc/pet. ether, to give imidazopyridinone 96 (46 mg, 12%) as a red solid: mp 140-142° C.; ¹H NMR (CDCl₃) δ 7.93 (d, J=0.5 Hz, 1H, H-4), 7.22 (d, J=8.6 Hz, 1H, H-6′), 6.84 (d, J=2.9 Hz, 1H, H-3′), 6.77 (dd, J=8.6, 2.9 Hz, 1 H, H-5′), 6.11 (d, J=0.5 Hz, 1H, H-7), 5.98 (br s, 1H, 6-NH), 4.63-4.70 (m, 2H, 1-CH, 3-CH), 3.82 (s, 3H, 4′-OCH₃), 2.26 (s, 3H, 2′-CH₃), 1.88-1.96 (m, 4H, 2×CH₂), 1.70-1.78 (m, 2 H, CH₂), 1.57-1.66 (m, 2H, CH₂), 1.51 (d, J=7.0 Hz, 6 H, 2×CH₃); MS m/z 381.2 (MH⁺, 100%). HRMS calcd for C₂₂H₂₈N₄O₂ (MH⁺) m/z 381.2285, found 381.2295 (−2.7 ppm). HPLC purity 97.7%.

### Example 86: SN39789 1-Cyclopentyl-6-((4-methoxy-2-methylphenyl)amino)-3-(2-methoxyethyl)-1,3-dihydro-2H-imidazo[4,5-c]pyridin-2-one (97)

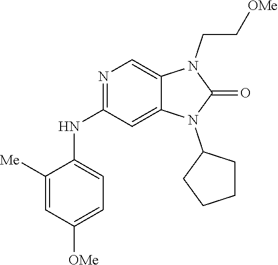 [SMILES: COCCn1c(=O)n(C2CCCC2)c2cc(Nc3ccc(OC)cc3C)ncc21]

6-Chloro-1-cyclopentyl-3-(2-methoxyethyl)-1,3-dihydro-2H-imidazo[4,5-c]pyridin-2-one (92). NaH (60% dispersion, 65 mg, 1.6 mmol) was added to a stirred solution of pyridinone 5 (0.32 g, 1.4 mmol) and 1-bromo-2-methoxyethane (0.19 mL, 2.0 mmol) in dry DMF (5 mL) at 5° C. The mixture was stirred at 20° C. for 16 h and then quenched with ice/water (5 mL) and the mixture was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (40-50%) of EtOAc/pet. ether, to give chloride 92 (301 mg, 75%) as a white solid: mp 80-83° C.; ¹H NMR (CDCl₃) δ 8.14 (s, 1H, H-4), 6.97 (d, J=0.4 Hz, 1H, H-7), 4.80 (pent, J=8.7 Hz, 1H, 1-CH), 4.05 (dd, J=5.3, 5.0 Hz, 2 H, CH₂O), 3.66 (dd, J=5.3, 5.0 Hz, 2 H, 3-CH₂), 3.33 (s, 3H, OCH₃), 1.91-2.09 (m, 6H, 3×CH₂), 1.69-1.79 (m, 2H, CH₂); MS m/z 296.2 (MH⁺, 100%), 298.2 (MH⁺, 35%). HRMS calcd for C₁₄H₁₉₃₅ClN₃O₂ (MH⁺) m/z 296.1160, found 296.1166 (−1.9 ppm).

1-Cyclopentyl-6-((4-methoxy-2-methylphenyl)amino)-3-(2-methoxyethyl)-1,3-dihydro-2H-imidazo[4,5-c]pyridin-2-one (97). A degassed mixture of chloride 92 (217 mg, 0.73 mmol), 4-methoxy-2-methylaniline (120 mg, 0.88 mmol), Pd₂dba₃ (33 mg, 37 μmol), XPhos (70 mg, 146 μmol) and Cs₂CO₃ (523 mg, 1.61 mmol) in MeCN (8 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with a gradient (60-100%) of EtOAc/pet. ether, to give imidazopyridinone 97 (122 mg, 42%) as a red foam: ¹H NMR (CDCl₃) δ 7.90 (d, J=0.5 Hz, 1H, H-4), 7.22 (d, J=8.6 Hz, 1H, H-6′), 6.83 (d, J=2.9 Hz, 1H, H-3′), 6.76 (dd, J=8.6, 3.0 Hz, 1H, H-5′), 6.10 (d, J=0.5 Hz, 1H, H-7), 5.98 (br s, 1H, 6-NH), 4.67 (pent, J=8.7 Hz, 1H, 1-CH), 3.99 (dd, J=5.4, 5.3 Hz, 2 H, CH₂O), 3.82 (s, 3H, 4′-OCH₃), 3.66 (dd, J=5.4, 5.3 Hz, 2 H, 3-CH₂), 3.34 (s, 3H, OCH₃), 2.25 (s, 3H, 2′-CH₃), 1.89-1.97 (m, 4H, 2×CH₂), 1.70-1.79 (m, 2H, CH₂), 1.57-1.67 (m, 2H, CH₂); MS m/z 397.2 (MH⁺, 100%); HRMS calcd for C₂₂H₂₉N₄O₃ (MH⁺) m/z 397.2234, found 397.2244 (−2.5 ppm). HPLC purity 96.8%.

### Example 87: SN39793 3-(2-(Benzyloxy)ethyl)-1-cyclopentyl-6-((4-methoxy-2-methylphenyl)amino)-1,3-dihydro-2H-imidazo[4,5-c]pyridin-2-one (98)

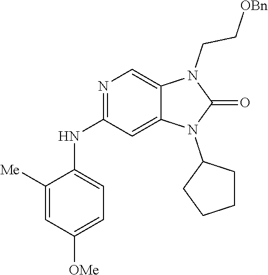 [SMILES: COc1ccc(Nc2cc3c(cn2)n(CCOCc2ccccc2)c(=O)n3C2CCCC2)c(C)c1]

3-(2-(Benzyloxy)ethyl)-6-chloro-1-cyclopentyl-1,3-dihydro-2H-imidazo[4,5-c]pyridin-2-one (93). NaH (60% dispersion, 90 mg, 2.3 mmol) was added to a stirred solution of pyridinone 5 (0.45 g, 1.9 mmol) and benzyl 2-bromoethyl ether (0.45 mL, 2.8 mmol) in dry DMF (5 mL) at 5° C. The mixture was stirred at 20° C. for 16 h and then quenched with ice/water (5 mL) and the mixture was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (20-50%) of EtOAc/pet. ether, to give chloride 93 (656 mg, 94%) as white crystals: mp 133-135° C.; ¹H NMR (CDCl₃) δ 8.14 (d, J=0.5 Hz, 1H, H-4), 7.24-7.31 (m, 3H, aryl-H), 7.17-7.20 (m, 2H, aryl-H), 6.97 (d, J=0.5 Hz, 1H, H-7), 4.78 (pent, J=8.7 Hz, 1H, 1-CH), 4.49 (s, 3H, CH₂O), 4.07 (dd, J=5.3, 5.0 Hz, 2 H, CH₂O), 3.75 (dd, J=5.3, 5.0 Hz, 2 H, 3-CH₂), 1.92-2.07 (m, 6H, 3×CH₂), 1.70-1.78 (m, 2H, CH₂); MS m/z 372.2 (MH⁺, 100%), 374.2 (MH⁺, 35%). HRMS calcd for C₂₀H₂₃³⁵ClN₃O₂ (MH⁺) m/z 372.1473, found 372.1477 (−1.0 ppm). 3-(2-(Benzyloxy)ethyl)-1-cyclopentyl-6-((4-methoxy-2-methylphenyl)amino)-1,3-dihydro-2H-imidazo[4,5-c]pyridin-2-one (98). A degassed mixture of chloride 93 (614 mg, 1.65 mmol), 4-methoxy-2-methylaniline (272 mg, 1.98 mmol), Pd₂dba₃ (75 mg, 82 μmol), XPhos (157 mg, 330 μmol) and Cs₂CO₃ (1.18 g, 3.63 mmol) in MeCN (12 mL) was stirred in a sealed tube at 120° C. for 16 h. The mixture was cooled, diluted with EtOAc (50 mL), filtered through diatomaceous earth and the filtrate was evaporated. The residue was partitioned between EtOAc (100 ml) and water (50 mL). The organic fraction was washed with water (30 mL), washed with brine (30 mL), dried (MgSO₄), filtered and the solvent evaporated. The residue was purified by chromatography, eluting with a gradient (40-60%) of EtOAc/pet. ether, to give imidazopyridinone 98 (308 mg, 39%) as a tan powder: mp (EtOAc/pet ether) 126-127° C.; ¹H NMR (CDCl₃) δ 7.90 (d, J=0.5 Hz, 1H, H-4), 7.21-7.35 (m, 6H, H-6′, 5×aryl-H), 6.84 (d, J=2.9 Hz, 1H, H-3′), 6.77 (dd, J=8.6, 3.0 Hz, 1H, H-5′), 6.10 (d, J=0.6 Hz, 1H, H-7), 5.98 (br s, 1H, 6-NH), 4.67 (pent, J=8.7 Hz, 1H, 1-CH), 4.52 (s, 2H, CH₂O), 4.02 (t, J=5.5 Hz, 2 H, CH₂O), 3.81 (s, 3H, 4′-OCH₃), 3.74 (t, J=5.5 Hz, 2 H, 3-CH₂), 2.26 (s, 3H, 2′-CH₃), 1.89-1.97 (m, 4H, 2×CH₂), 1.70-1.80 (m, 2H, CH₂), 1.57-1.66 (m, 2H, CH₂); MS m/z 473.2 (MH⁺, 100%). HRMS calcd for C₂₈H₃₃N₄O₃ (MH⁺) m/z 473.2547, found 473.2557 (−2.0 ppm). HPLC purity 99.7%.

### Example 88: SN39794 1-Cyclopentyl-3-(2-hydroxyethyl)-6-((4-methoxy-2-methylphenyl)amino)-1,3-dihydro-2H-imidazo[4,5-c]pyridin-2-one (99)

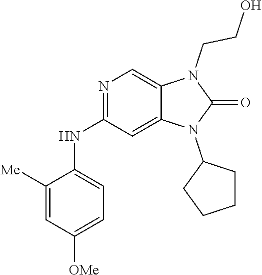 [SMILES: COc1ccc(Nc2cc3c(cn2)n(CCO)c(=O)n3C2CCCC2)c(C)c1]

A mixture of benzyl ether 98 (255 mg, 0.54 mmol) and Pd/C (30 mg) in HOAc (50 mL) was stirred vigorously under H₂ (60 psi) for 24 h. The mixture was filtered through diatomaceous earth, washed with EtOAc (20 mL). The solvent was evaporated and the residue was dissolved in EtOAc (80 mL) and washed sequentially with NaHCO₃(50 mL), water (50 mL) and brine (30 mL). The organic fraction was dried (MgSO₄) and the solvent evaporated. The residue was purified by chromatography, eluting with EtOAc, to give alcohol 99 (179 mg, 87%) as a white powder: mp 159-161° C. ¹H NMR (CDCl₃) δ 7.85 (d, J=0.5 Hz, 1H, H-4), 7.12 (d, J=8.6 Hz, 1 H, H-6′), 6.84 (d, J=2.9 Hz, 1H, H-3′), 6.77 (d, J=8.6, 2.9 Hz, 1H, H-5′), 6.09 (d, J=0.6 Hz, 1H, H-7), 6.04 (br s, 1H, 6-NH), 4.67 (pent, J=8.7 Hz, 1H, 1-CH), 3.95-4.00 (m, 4H, 3-CH₂, CH₂O), 3.82 (s, 3H, 4′-OCH₃), 2.24 (s, 3H, 2′-CH₃), 1.88-1.98 (m, 4H, 2×CH₂), 1.70-1.78 (m, 2H, CH₂), 1.58-1.68 (m, 2H, CH₂), OH not observed; MS m/z 383.2 (MH⁺, 100%). HRMS calcd for C₂₁H₂₇N₄O₃ (MH⁺) m/z 383.2078, found 383.2082 (−1.7 ppm). HPLC purity 99.4%.

### Example 89: SN39478 6-((4-Methoxy-2-methylphenyl)amino)-1-(2-methoxyethyl)-3-methyl-1,3-dihydro-2H-imidazo[4,5-c]pyridin-2-one (104)

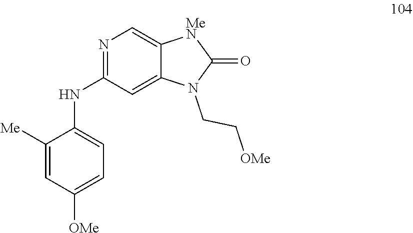 [SMILES: COCCn1c(=O)n(C)c2cnc(Nc3ccc(OC)cc3C)cc21]

2-Chloro-N-(2-methoxyethyl)-5-nitropyridin-4-amine (100). A solution of 2-methoxyethylamine (0.49 mL, 5.66 mmol) in dry DCM (5 mL) was added dropwise to a stirred solution of nitropyridine 2 (1.04 g, 5.39 mmol) and iPr₂NEt (1.00 mL, 5.93 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (10-20%) of EtOAc/pet. ether, to give chloride 100 (1.19 g, 95%) as yellow needles: mp 84-86° C.; ¹H NMR (CDCl₃) δ 9.02 (s, 1H, H-6), 8.36 (br s, 1H, 4-NH), 6.77 (s, 1H, H-3), 3.67 (dd, J=5.4, 5.0 Hz, 2 H, H-2′), 3.49 (dt, J=5.3, 5.1 Hz, 2 H, H-1′), 3.44 (s, 3H, 2′-OCH₃); MS m/z 232.2 (MH⁺, 100%), 234.2 (MH⁺, 35%). Analysis calcd for CsH1₀ClN₃03: C, 41.48; H, 4.35; N, 18.18. Found: C, 41.65; H, 4.24; N, 18.18%.

6-Chloro-N⁴-(2-methoxyethyl)pyridine-3,4-diamine (101). A solution of nitropyridine 100 (1.14 g, 4.95 mmol) in EtOAc (50 ml) was added dropwise to a stirred suspension of SnCl₂·2H₂O (4.47 g, 19.8 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 101 as a white powder: mp 130-131° C.; ¹H NMR (CDCl₃) δ 7.65 (s, 1H, H-2), 6.45 (s, 1H, H-5), 4.60 (br s, 1H, 4-NH), 3.64 (dd, J=5.3, 5.0 Hz, 2 H, H-2′), 3.40 (s, 3H, 2′-OCH₃), 3.31 (dt, J=5.3, 5.1 Hz, 2 H, H-1′), 3.06 (br s, 2H, 3-NH₂); MS m/z 202.2 (MH⁺, 100%), 204.1 (MH⁺, 35%). Analysis calcd for C₈H₁₂ClN₃O: C, 47.65; H, 6.00; N, 20.84. Found: C, 47.42; H, 6.03; N, 21.10%.

6-Chloro-1-(2-methoxyethyl)-1,3-dihydro-2H-imidazo[4,5-c]pyridin-2-one (102). CDI (0.96 g, 5.89 mmol) was added to a stirred solution of diamine 101 (0.99 g, 4.90 mmol) in dry MeCN (50 mL) at 20° C. The mixture was stirred at 20° C. for 48 h. The solvent was evaporated and the residue partitioned between CHCl₃ (100 mL) and water (100 mL). The organic fraction was washed with water (2×50 mL), washed with brine (50 mL), dried (MgSO₄), filtered and the solvent evaporated. The residue was precipitated from 50% EtOAc/pet. ether, to give the pyridinone 102 (0.75 g, 67%) as a white powder: mp 170-171° C.; ¹H NMR (CDCl₃) δ 9.52 (br s, 1 H, 3-H), 8.10 (d, J=0.5 Hz, 1H, H-4), 7.12 (d, J=0.5 Hz, 1H, H-7), 4.03 (dd, J=5.2, 4.9 Hz, 2 H, H-2′), 3.68 (dd, J=5.2, 4.9 Hz, 2 H, H-1′), 3.34 (s, 3H, 2′-OCH₃); MS m/z 228.1 (MH⁺, 100%), 230.1 (MH⁺, 35%). Anal calcd for C₉H₁₀ClN₃O₂: C, 47.49; H, 4.43; N, 18.46. Found: C, 47.54; H, 4.28; N, 18.57%.

6-Chloro-1-(2-methoxyethyl)-3-methyl-1,3-dihydro-2H-imidazo[4,5-c]pyridin-2-one (103). NaH (60% dispersion, 152 mg, 3.80 mmol) was added to a stirred solution of pyridinone 102 (0.72 g, 3.16 mmol) and MeI (0.30 mL, 4.74 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 103 (0.43 g, 56%) as white powder: mp (DCM) 80-82° C.; ¹H NMR (CDCl₃) δ 7.96 (s, 1H, H-4), 7.09 (d, J=0.5 Hz, 1H, H-7), 4.02 (dd, J=5.3, 4.9 Hz, 1H, H-2′), 3.65 (dd, J=5.2, 4.9 Hz, 2 H, H-1′), 3.45 (s, 3H, 3-CH₃), 3.34 (s, 3H, 2′-OCH₃); MS m/z 242.2 (MH⁺, 100%), 244.1 (MH⁺, 35%). Anal calcd for C₁₀H₁₂ClN₃O₂·0.1CH₂C₁₂: C, 48.49; H, 4.92; N, 116.80 Found: C, 48.66; H, 4.95; N, 16.89%. 6-((4-Methoxy-2-methylphenyl)amino)-1-(2-methoxyethyl)-3-methyl-1,3-dihydro-2H-imidazo[4,5-c]pyridin-2-one (104). A degassed mixture of chloride 103 (117 mg, 0.48 mmol), aniline (80 mg, 0.58 mmol), Pd₂dba₃ (22 mg, 24 μmol), XPhos (46 mg, 96 μmol) and Cs₂CO₃ (344 mg, 1.06 mmol) in MeCN (6 mL) was stirred in a sealed tube at 120° C. for 16 h.

The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc/pet. ether, to give imidazopyridinone 104 (84 mg, 51%) as white needles: mp (EtOAc/pet ether) 133-135° C.; ¹H NMR (CDCl₃) δ 7.74 (d, J=0.4 Hz, 1H, H-4), 7.22 (d, J=8.6 Hz, 1H, H-6″), 6.83 (d, J=2.9 Hz, 1H, H-3″), 6.76 (dd, J=8.6, 2.9 Hz, 1H, H-5″), 6.15 (d, J=0.4 Hz, 1H, H-7), 5.98 (s, 1H, 6-NH), 3.88 (t, J=5.3 Hz, 1H, H-2′), 3.82 (s, 3H, 4″-OCH₃), 3.57 (t, J=5.3 Hz, 2 H, H-1′), 3.39 (s, 3H, 3-CH₃), 3.26 (s, 3H, 2′-OCH₃), 2.24 (s, 3H, 2″-CH₃); MS m/z 343.2 (MH⁺, 100%). Anal calcd for C₁₈H₂₂N₄O₃: C, 63.14; H, 6.48; N, 16.36. Found: C, 63.25; H, 6.38; N, 16.45%. HPLC purity 99.8%.

### Example 90: SN39551 6-((4-Chloro-2-methylphenyl)amino)-1-(2-methoxyethyl)-3-methyl-1,3-dihydro-2H-imidazo[4,5-c]pyridin-2-one (105)

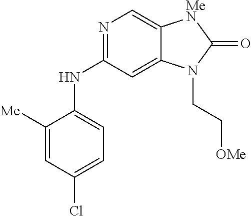 [SMILES: COCCn1c(=O)n(C)c2cnc(Nc3ccc(Cl)cc3C)cc21]

A degassed mixture of chloride 103 (110 mg, 0.46 mmol), 4-chloro-2-methylaniline (77 mg, 0.55 mmol), Pd₂dba₃ (21 mg, 23 μmol), XPhos (44 mg, 92 μmol) and Cs₂CO₃ (330 mg, 1.01 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (80-100% EtOAc/pet. ether) to give imidazopyridinone 105 (107 mg, 68%) as a cream powder: mp (EtOAc/pet ether) 92-95° C.; ¹H NMR (CDCl₃) δ 7.79 (d, J=0.6 Hz, 1H, H-4), 7.36 (d, J=8.5 30 Hz, 1H, H-6″), 7.22 (d, J=2.5 Hz, 1H, H-3″), 7.15 (dd, J=8.5, 2.5 Hz, 1H, H-5″), 6.47 (d, J=0.6 Hz, 1H, H-7), 6.08 (s, 1H, 6-NH), 3.94 (dd, J=5.3, 5.0 Hz, 2 H, H-2′), 3.61 (dd, J=5.3, 5.0 Hz, 2 H, H-1′), 3.41 (s, 3H, 2′-OCH₃), 3.30 (s, 3H, 3-CH₃), 2.26 (s, 3H, 2″-CH₃); MS m/z 347.1 (MH⁺, 100%), 349.2 (MH⁺, 35%). HRMS calcd for C₁₇H₂O³⁵ClN₄O₂ (MH⁺) m/z 347.1269, found 347.1276 (−1.9 ppm); calcd for C₁₇H₂₀³⁷ClN₄O₂ (MH⁺) m/z 349.1246, found 349.1249 (−1.0 ppm). HPLC purity 96.9%.

### Example 91: SN39887 6-((4-Methoxy-2-methylphenyl)amino)-3-methyl-1-(oxetan-3-yl)-1,3-dihydro-2H-imidazo[4,5-c]pyridin-2-one (110)

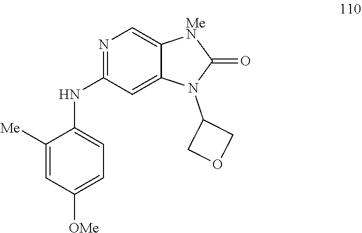 [SMILES: COc1ccc(Nc2cc3c(cn2)n(C)c(=O)n3C2COC2)c(C)c1]

2-Chloro-5-nitro-N-(oxetan-3-yl)pyridin-4-amine (106). A solution of oxetan-3-amine (0.36 g, 5.0 mmol) in dry DCM (5 mL) was added dropwise to a stirred solution of nitropyridine 2 (0.87 g, 4.50 mmol) and iPr₂NEt (1.18 mL, 6.75 mmol) in dry DCM (100 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (20-40%) of EtOAc/pet. ether, to give chloride 106 (1.17 g, 90%) as a yellow powder: mp 139-141° C.; ¹H NMR (CDCl₃) δ 9.06 (s, 1H, H-6), 8.52 (br s, 1H, 4-NH), 6.42 (s, 1H, H-3), 5.08 (dd, J=7.2, 6.7 Hz, 2 H, H-2′, H-4′), 4.73-4.78 (m, 1H, H-3′), 4.67 (dd, J=6.4, 6.2 Hz, 2 H, H-2′, H-4′); MS m/z 230.1 (MH⁺, 100%), 232.0 (MH⁺, 35%); HRMS calcd for C₈H₉³⁵ClN₃O₃(MH⁺) m/z 230.0327, found 230.0321 (2.5 ppm).

6-Chloro-N⁴-(oxetan-3-yl)pyridine-3,4-diamine (107). A solution of nitropyridine 106 (1.17 g, 5.10 mmol) in EtOAc (50 ml) was added dropwise to a stirred suspension of SnCl₂·2H₂O (4.60 g, 20.4 mmol) in EtOAc (100 mL) at 50° C. while maintaining the temperature below 60° C. The mixture was stirred at 60° C. for 2 h and then cooled to 5° C. and conc. aq.

NH₃ solution added until the solution was basic (pH 9). The resulting precipitate was filtered and washed with EtOAc (100 mL). The combined organic fraction was dried (MgSO₄), filtered and the solvent evaporated to give diamine 107 (1.01 g, 99%) as a white powder: mp 183-186° C.; ¹H NMR [(CD₃)₂SO] δ 7.25 (s, 1H, H-2), 6.71 (br s, 1H, 3-NH₂), 6.34 (s, 1H, H-5), 5.57 (br s, 1H, 3-NH₂), 3.33-3.40 (m, 4H, 4-NH, H-2′, H-3′, H-4′), 3.13 (dt, J=9.1, 2.3 Hz, 1 H, H-2′), 2.95 (ddd, J=11.1, 4.7, 2.0 Hz, 1H, H-4′); MS m/z 200.1 (MH⁺, 100%), 202.1 (MH⁺, 35%); HRMS calcd for C₈H₁₁³⁵ClN₃O (MH⁺) m/z 200.0585, found 200.0589 (−2.1 ppm).

6-Chloro-1-(oxetan-3-yl)-1,3-dihydro-2H-imidazo[4,5-c]pyridin-2-one (108). CDI (0.97 g, 6.00 mmol) was added to a stirred solution of diamine 107 (1.00 g, 5.01 mmol) in dry MeCN (50 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was purified by chromatography, eluting with a gradient (80-100%) of EtOAc/pet. ether, to give the pyridinone 108 (0.24 g, 22%) as white crystals: mp 234° C. (decomp); ¹H NMR [(CD₃)₂SO] δ 7.74 (s, 1H, H-4), 7.69 (s, 1H, H-7), 6.55 (br d, J=4.0 Hz, 1H, 3-NH), 4.60-4.67 (m, 1H, H-3′), 4.12-4.22 (m, 2H, H-2′, H-4′), 3.62 (ddd, J=11.4, 4.3, 3 1 Hz, 1H, H-2′), 3.97-3.05 (m, 1H, H-4′); MS m/z 226.1 (MH⁺, 100%), 228.1 (MH⁺, 35%); HRMS calcd for C₉H₉³⁵ClN₃O₂ (MH⁺) m/z 226.0378, found 226.0374 (1.8 ppm).

6-Chloro-3-methyl-1-(oxetan-3-yl)-1,3-dihydro-2H-imidazo[4,5-c]pyridin-2-one (109). NaH (60% dispersion, 64 mg, 1.60 mmol) was added to a stirred solution of pyridinone 108 (0.30 g, 1.33 mmol) and MeI (0.12 mL, 2.00 mmol) in dry DMF (10 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 109 (0.16 g, 49%) as a white powder: mp 192-194° C.; ¹H NMR (CDCl₃) δ 7.98 (s, 1H, H-4), 7.75 (s, 1H, H-7), 4.66 (t, J=8.6 Hz, 1H, H-2′), 4.34-4.43 (m, 1H, H-3″), 4.12 (t, J=8.6 Hz, 1H, H-4′), 3.47 (dd, J=11.1, 3.3 Hz, 1H, H-2′), 3.15 (dd, J=11.1, 9.7 Hz, 1H, H-4′), 3.00 (s, 3H, 3-CH₃); MS m/z 240.0 (MH⁺, 100%), 242.0 (MH⁺, 35%); HRMS calcd for C₁₀H₁₁³⁵ClN₃O₂ (MH⁺) m/z 240.0534, found 240.0543 (−3.7 ppm). 6-((4-Methoxy-2-methylphenyl)amino)-3-methyl-1-(oxetan-3-yl)-1,3-dihydro-2H-imidazo[4,5-c]pyridin-2-one (110). A degassed mixture of chloride 109 (148 mg, 0.62 mmol), 4-methoxy-2-methylaniline (102 mg, 0.74 mmol), Pd₂dba₃ (28 mg, 31 μmol), XPhos (59 mg, 124 μmol) and Cs₂CO₃ (444 mg, 1.36 mmol) in MeCN (10 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 110 (101 mg, 48%) as a pink foam: mp (EtOAc/pet ether) 77-80° C.; ¹H NMR (CDCl₃) δ 7.63 (s, 1H, H-4), 7.31 (s, 1H, H-7), 7.30 (d, J=8.6 Hz, 1H, H-6″), 6.78 (d, J=2.9 Hz, 1H, H-3″), 6.74 (dd, J=8.6, 2.9 Hz, 1H, H-5″), 5.81 (s, 1H, 6-NH), 4.58 (t, J=8.6 Hz, 1H, H-2′ or H-4′), 4.38 (dq, J=8.5, 3.1 Hz, 1H, 1-CH), 4.05 (t, J=8.6 Hz, 1H, H-2′ or H-4′), 3.81 (s, 3H, 4″-OCH₃), 3.33 (dd, J=10.9, 3.2 Hz, 1H, H-2′ or H-4′), 3.00 (dd, J=10.8, 10.0 Hz, 1H, H-2′ or H-4′), 2.90 (s, 3H, 3-CH₃), 2.24 (s, 3H, 2″-CH₃); MS m/z 341.2 (MH⁺, 100%); HRMS calcd for C₁₈H₂₁N₄O₃ (MH⁺) m/z 341.1608, found 341.1612 (−1.0 ppm). HPLC purity 98.0%.

### Example 92: SN39878 6-((4-Methoxy-2-methylphenyl)amino)-3-methyl-1-(tetrahydrofuran-3-yl)-1,3-dihydro-2H-imidazo[4,5-c]pyridin-2-one (115)

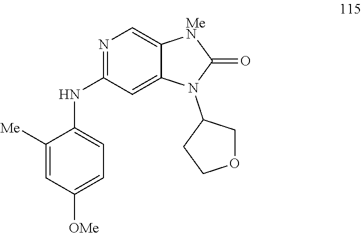 [SMILES: COc1ccc(Nc2cc3c(cn2)n(C)c(=O)n3C2CCOC2)c(C)c1]

2-Chloro-5-nitro-N-(tetrahydrofuran-3-yl)pyridin-4-amine (111). iPr₂NEt (2.62 mL, 15.1 mmol) was added dropwise to a stirred solution of nitropyridine 2 (1.17 g, 6.02 mmol) and tetrahydrofuran-3-amine. HCl (0.78 g, 6.3 mmol) in dry DCM (80 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (40-50%) of EtOAc/pet. ether, to give chloride 111 (1.37 g, 93%) as yellow crystals: mp 132-134° C.; ¹H NMR (CDCl₃) δ 9.03 (s, 1H, H-6), 8.27 (br s, 1H, 4-NH), 6.73 (s, 1H, H-3), 4.18-4.25 (m, 1H, H-3′), 4.00-4.08 (m, 2H, H-2′), 3.93 (ddd, J=8.8, 8.6, 5.4 Hz, 1H, H-5′), 3.84 (dd, J=9.7, 2.8 Hz, 1H, H-5′), 2.39-2.49 (m, 1H, H-4′), 1.95-2.03 (m, 1H, H-4′); MS m/z 244.1 (MH⁺, 100%), 246.1 (MH⁺, 35%); HRMS calcd for C₉H₁₁³⁵ClN₃O₃(MH⁺) m/z 244.0484, found 244.04387(−1.3 ppm).

6-Chloro-N₄-(tetrahydrofuran-3-yl)pyridine-3,4-diamine (112). A solution of nitropyridine 111 (1.36 g, 5.80 mmol) in EtOAc (50 ml) was added dropwise to a stirred suspension of SnCl₂·2H₂O (5.05 g, 22.4 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 112 (1.17 g, 94%) as a white powder: ¹H NMR [(CD₃)₂SO] δ 7.66 (s, 1H, H-2), 6.43 (s, 1H, H-5), 4.46 (br d, J=5.3 Hz, 1 H, 3-NH), 4.04-4.10 (m, 1H, H-3′), 3.94-4.02 (m, 2H, H-2′), 3.88 (dt, J=8.6, 5.4 Hz, 1H, H-5′), 3.78 (dd, J=9.4, 2.4 Hz, 1H, H-5′), 3.04 (br s, 2H, 4-NH₂), 2.29-2.38 (m, 1H, H-4′), 1.87-1.95 (m, 1H, H-4′); MS m/z 214.1 (MH⁺, 100%), 216.1 (MH⁺, 35%); HRMS calcd for C₉H₁₃³⁵ClN₃O (MH⁺) m/z 214.0742, found 214.0739 (1.2 ppm).

6-Chloro-1-(tetrahydrofuran-3-yl)-1,3-dihydro-2H-imidazo[4,5-c]pyridin-2-one (113). CDI (0.44 g, 2.71 mmol) was added to a stirred solution of diamine 112 (0.48 g, 2.25 mmol) in dry MeCN (50 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was precipitated from 50% EtOAc/pet. ether, to give the pyridinone 113 (0.45 g, 83%) as a white powder: mp 254-256° C.; ¹H NMR [(CD₃)₂SO] δ 11.42 (br s, 1H, 3-H), 7.97 (s, 1H, H-4), 7.28 (s, 1H, H-7), 5.02-5.10 (m, 1H, H-3′), 4.18 (dt, J=8.5, 4.3 Hz, 1H, H-5′), 3.98 (dd, J=9.9, 3.6 Hz, 1H, H-2′), 3.82 (dd, J=9.9, 7.5 Hz, 1H, H-2′), 3.66 (q, J=8.3 Hz, 1H, H-5′), 2.27-2.40 (m, 1H, H-4′), 2.00-2.10 (m, 1H, H-4′); MS m/z 240.1 (MH⁺, 100%), 242.1 (MH⁺, 35%); HRMS calcd for C₁₀H₁₁³⁵ClN₃O₂ (MH⁺) m/z 240.0534, found 240.0535 (−0.3 ppm).

6-Chloro-3-methyl-1-(tetrahydrofuran-3-yl)-1,3-dihydro-2H-imidazo[4,5-c]pyridin-2-one (114). NaH (60% dispersion, 151 mg, 3.78 mmol) was added to a stirred solution of pyridinone 113 (0.76 g, 3.15 mmol) and MeI (0.29 mL, 4.73 mmol) in dry DMF (10 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with EtOAc, to give chloride 114 (0.54 g, 68%) as a white powder: mp 154-156° C.; ¹H NMR (CDCl₃) δ 8.00 (s, 1H, H-4), 7.32 (d, J=0.4 Hz, 1H, H-7), 5.23-5.29 (m, 1H, H-3′), 4.33 (dt, J=8.8, 3.5 Hz, 1H, H-5′), 4.09 (dd, J=10.4, 3.0 Hz, 1H, H-2′), 3.92 (dd, J=10.4, 7.5 Hz, 1H, H-2′), 3.76 (dt, J=9.2, 7.4 Hz, 1H, H-5′), 3.45 (s, 3H, 3-CH₃), 2.42-2.52 (m, 1H, H-4′), 2.03-2.14 (m, 1H, H-4′); MS m/z 254.0 (MH⁺, 100%), 256.0 (MH⁺, 35%); HRMS calcd for C₁₁H₁₃³⁵ClN₃O₂ (MH⁺) m/z 254.0691, found 254.0686 (1.9 ppm). 6-((4-Methoxy-2-methylphenyl)amino)-3-methyl-1-(tetrahydrofuran-3-yl)-1,3-dihydro-2H-imidazo[4,5-c]pyridin-2-one (115). A degassed mixture of chloride 114 (112 mg, 0.41 mmol), 4-methoxy-2-methylaniline (73 mg, 0.53 mmol), Pd₂dba₃ (19 mg, 21 μmol), XPhos (39 mg, 82 μmol) and Cs₂CO₃ (294 mg, 0.90 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 115 (104 mg, 66%) as a tan foam: mp 113-116° C.; ¹H NMR [(CD₃)₂SO] δ 7.78 (s, 1H, H-4), 7.73 (s, 1H, 6-NH), 7.27 (d, J=8.7 Hz, 1H, H-6″), 6.79 (d, J=2.9 Hz, 1H, H-3″), 6.70 (dd, J=8.7, 2.9 Hz, 1H, H-5″), 6.51 (s, 1H, H-7), 5.00-5.07 (m, 1H, H-3′), 4.04 (dt, J=8.5, 4.4 Hz, 1 H, H-5′), 3.88 (dd, J=9.7, 4.0 Hz, 1H, H-2′), 3.80 (dd, J=9.7, 7.6 Hz, 1H, H-2′), 3.72 (s, 3H, 4″-OCH₃), 3.66 (q, J=8.2 Hz, 1H, H-5′), 3.35 (s, 3H, 3-CH₃), 2.25-2.35 (m, 1H, H-4′), 2.16 (s, 3H, 2″-CH₃), 2.00-2.08 (m, 1H, H-4′); MS m/z 355.2 (MH⁺, 100%); HRMS calcd for C₁₉H₂₃N₄O₃ (MH⁺) m/z 355.1765, found 355.1785 (−5.7 ppm). HPLC purity 99.4%.

### Example 93: SN39881 6-((4-Chloro-2-methylphenyl)amino)-3-methyl-1-(tetrahydrofuran-3-yl)-1,3-dihydro-2H-imidazo[4,5-c]pyridin-2-one (116)

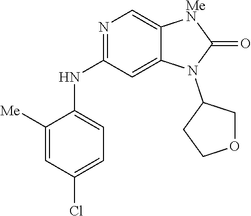 [SMILES: Cc1cc(Cl)ccc1Nc1cc2c(cn1)n(C)c(=O)n2C1CCOC1]

A degassed mixture of chloride 114 (118 mg, 0.47 mmol), 4-chloro-2-methylaniline (79 mg, 0.56 mmol), Pd₂dba₃ (21 mg, 23 μmol), XPhos (44 mg, 93 μmol) and Cs₂CO₃ (333 mg, 1.02 mmol) in MeCN (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 116 (134 mg, 80%) as a white powder: mp (EtOAc/pet ether) 153-156° C.; ¹H NMR [(CD₃)₂SO] δ 7.94 (s, 1H, H-4), 7.87 (s, 1H, 6-NH), 7.65 (d, J=8.7 Hz, 1H, H-6″), 7.21 (d, J=2.6 Hz, 1H, H-3″), 7.13 (dd, J=8.7, 2.6 Hz, 1H, H-5″), 6.79 (s, 1H, H-7), 5.03-5.10 (m, 1H, H-3′), 4.13 (dt, J=8.5, 4.3 Hz, 1H, H-5′), 3.92 (dd, J=9.7, 4.1 Hz, 1H, H-2′), 3.84 (dd, J=9.7, 7.7 Hz, 1H, H-2′), 3.69 (q, J=8.2 Hz, 1H, H-5′), 3.28 (s, 3H, 3-CH₃), 2.27-2.37 (m, 1H, H-4′), 2.22 (s, 3H, 2″-CH₃), 2.02-2.12 (m, 1H, H-4′); MS m/z 359.2 (MH⁺, 100%), MS m/z 361.2 (MH⁺, 35%); HRMS calcd for C₁₈H₂₀³⁵ClN₄O² (MH⁺) m/z 359.1269, found 359.1290 (−5.7 ppm). HPLC purity 94.4% Example 94: SN39536 6-((4-Methoxy-2-methylphenyl)amino)-3-methyl-1-(tetrahydro-2H-pyran-4-yl)-1,3-dihydro-2H-imidazo[4,5-c]pyridin-2-one (121).

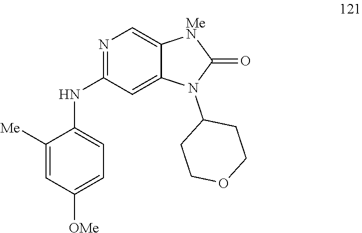 [SMILES: COc1ccc(Nc2cc3c(cn2)n(C)c(=O)n3C2CCOCC2)c(C)c1]

2,4-Dichloro-5-nitropyridine (2). A mixture of 4-chloro-5-nitropyridin-2-ol (5.38 g, 30.8 mmol), POCl₃ (60 mL) and tetramethylammonium chloride (10.1 g, 32.5 mmol) was stirred at 120° C. for 3 h. The mixture was cooled, poured into ice/water (500 mL) and stirred for 1 h at 0-10° C. The mixture was extracted with DCM (3×100 mL), the combined organic extract dried (MgSO₄). The solution was filtered through a short column of neutral alumina, washing with DCM (50 mL). The solvent was evaporated to give nitropyridine 2 as a clear oil (5.43 g, 91%): ¹H NMR (CDCl₃) δ 8.97 (s, 1H, H-6), 7.59 (s, 1H, H-3); MS m/z 192.9 (MH⁺, 100%), 194.9 (MH⁺, 70%).

2-Chloro-5-nitro-N-(tetrahydro-2H-pyran-4-yl)pyridin-4-amine (117). A solution of tetrahydro-2H-pyran-4-amine (0.55 g, 5.4 mmol) in dry DCM (5 mL) was added dropwise to a stirred solution of nitropyridine 2 (1.00 g, 5.18 mmol) and iPr₂NEt (1.15 mL, 6.22 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (30-50%) of EtOAc/pet. ether, to give chloride 117 (1.12 g, 84%) as yellow needles: mp 169-171° C.; ¹H NMR (CDCl₃) δ 9.04 (s, 1H, H-6), 8.19 (br s, 1H, 4-NH), 6.75 (s, 1H, H-3), 4.04 (ddd, J=12.0, 3.8, 3.6 Hz, 2 H, H-2′, H-6′), 3.65-3.76 (m, 1H, H-4′), 3.58 (dt, J=10.9, 2.3 Hz, 2 H, H-2′, H-6′), 2.06 (br d, J=11.6 Hz, 2 H, H-3′, H-5′), 1.70 (ddd, J=10.5, 4.2, 3.0 Hz, 2 H, H-3′, H-5′); MS m/z 256.0 (MH⁺, 100%), 258.0 (MH⁺, 35%); HRMS calcd for C₁₀H₁₃³⁵ClN₃O₃(MH⁺) m/z 256.0640, found 256.0638 (0.8 ppm).

6-Chloro-N⁴-(tetrahydro-2H-pyran-4-yl)pyridine-3,4-diamine (118). A solution of nitropyridine 117 (1.08 g, 4.19 mmol) in EtOAc (30 ml) was added dropwise to a stirred suspension of SnCl₂·2H₂O (3.78 g, 16.8 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 118 as a white powder: mp 139-141° C.; ¹H NMR [(CD₃)₂SO] δ 7.67 (s, 1H, H-2), 6.46 (s, 1H, H-5), 4.27 (br d, J=6.8 Hz, 1 H, 4-NH), 4.03 (ddd, J=11.7, 3.6, 3.3 Hz, 2 H, H-2′, H-6′), 3.46-3.57 (m, 3H, H-2′, H-4′, H-6′), 2.99 (br s, 2H, 3-NH₂), 2.02 (br d, J=12.4 Hz, 2 H, H-3′, H-5′), 1.50-1.61 (m, 2H, H-3′, H-5′); MS m/z 228.1 (MH⁺, 100%), 230.1 (MH⁺, 35%); HRMS calcd for C₁₀H₁₅³⁵ClN₃O (MH⁺) m/z 228.0898, found 258.0896 (1.1 ppm). Anal. calcd for C₁₀H₁₄ClN₃O·¼EtOAc: C, 52.91; H, 6.46; N, 16.83. Found: C, 52.91; H, 6.58; N, 16.90%.

6-Chloro-1-(tetrahydro-2H-pyran-4-yl)-1,3-dihydro-2H-imidazo[4,5-c]pyridin-2-one (119). CDI (0.74 g, 4.54 mmol) was added to a stirred solution of diamine 118 (0.94 g, 4.13 mmol) in dry MeCN (50 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was precipitated from 50% EtOAc/pet. ether, to give the pyridinone 119 (0.83 g, 84%) as a white powder: mp 281-283° C.; ¹H NMR (CDCl₃) δ 9.13 (br s, 1H, 3-H), 8.13 (s, 1H, H-4), 7.19 (s, 1H, H-7), 4.54 (tt, J=12.5, 4.4 Hz, 1H, H-4′), 4.17 (dd, J=11.7, 4.6 Hz, 2 H, H-2′, H-6′), 3.56 (dt, J=12.0, 1.8 Hz, 2 H, H-2′, H-6′), 2.40 (dq, J=12.6, 4.6 Hz, 2 H, H-3′, H-5′), 1.86 (ddd, J=12.6, 3.9, 1.3 Hz, 2 H, H-3′, H-5′); MS m/z 254.1 (MH⁺, 100%), 256.1 (MH⁺, 35%). Anal calcd for C₁₁H₁₂ClN₃O₂: C, 52.08; H, 4.77; N, 16.56. Found: C, 52.16; H, 4.77; N, 16.23%.

6-Chloro-3-methyl-1-(tetrahydro-2H-pyran-4-yl)-1,3-dihydro-2H-imidazo[4,5-c]pyridin-2-one (120). NaH (60% dispersion, 163 mg, 4.07 mmol) was added to a stirred solution of pyridinone 119 (0.86 g, 3.39 mmol) and MeI (0.32 mL, 5.09 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 120 (0.43 g, 47%) as white crystals: mp 190-192° C.; ¹H NMR (CDCl₃) δ 7.99 (s, 1H, H-4), 7.15 (d, J=0.5 Hz, 1H, H-7), 4.54 (tt, J=12.5, 4.4 Hz, 1H, H-4′), 4.15 (dd, J=11.7, 4.7 Hz, 2 H, H-2′, H-6′), 3.55 (dt, J=12.0, 1.9 Hz, 2 H, H-2′, H-6′), 3.45 (s, 3H, 3-CH₃), 2.38 (dq, J=12.5, 4.7 Hz, 2 H, H-3′, H-5′), 1.77 (ddd, J=12.4, 4.0, 1.5 Hz, 2 H, H-3′, H-5′); MS m/z 268.0 (MH⁺, 100%), 270.0 (MH⁺, 35%); HRMS calcd for C₁₂H₁₅³⁵ClN₃O₂ (MH⁺) m/z 268.0847, found 268.0854 (−2.6 ppm). 6-((4-Methoxy-2-methylphenyl)amino)-3-methyl-1-(tetrahydro-2H-pyran-4-yl)-1,3-dihydro-2H-imidazo[4,5-c]pyridin-2-one (121). A degassed mixture of chloride 120 (125 mg, 0.47 mmol), aniline (77 mg, 0.56 mmol), Pd₂dba₃ (21 mg, 24 μmol), XPhos (45 mg, 94 μmol) and Cs₂CO₃ (337 mg, 1.03 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (80-100%) of EtOAc/pet. ether, to give imidazopyridinone 121 (122 mg, 70%) as pink crystals: mp (EtOAc/pet ether) 176-178° C.; 1H NMR (CDCl₃) δ 7.77 (s, 1H, H-4), 7.25 (d, J=8.6 Hz, 1H, H-6″), 6.85 (d, J=2.9 Hz, 1H, H-3″), 6.78 (dd, J=8.6, 2.9 Hz, 1H, H-5″), 6.24 (d, J=0.5 Hz, 1H, H-7), 5.95 (s, 1H, 6-NH), 4.41 (tt, J=12.4, 4.2 Hz, 1H, H-4′), 4.07 (dd, J=11.6, 4.4 Hz, 2 H, H-2′, H-6′), 3.83 (s, 3H, 4″-OCH₃), 3.50 (dt, J=12.0, 1.6 Hz, 2 H, H-2′, H-6′), 3.38 (s, 3H, 3-CH₃), 2.20-2.33 (m, 5H, 2″-CH₃, H-3′, H-5′), 1.69 (dd, J=12.4, 2.4 Hz, 2 H, H-3′, H-5′); MS m/z 369.2 (MH⁺, 100%). Anal calcd for C₂₀H₂₄N₄O₃.% EtOAc: C, 64.60; H, 6.71; N, 14.35. Found: C, 64.76; H, 6.74; N, 14.48%. HPLC purity 99.4%.

### Example 95: SN39537 6-((4-Chloro-2-methylphenyl)amino)-3-methyl-1-(tetrahydro-2H-pyran-4-yl)-1,3-dihydro-2H-imidazo[4,5-c]pyridin-2-one (122)

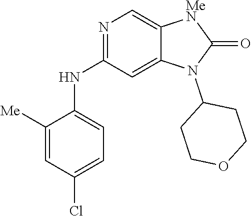 [SMILES: Cc1cc(Cl)ccc1Nc1cc2c(cn1)n(C)c(=O)n2C1CCOCC1]

A degassed mixture of chloride 120 (107 mg, 0.40 mmol), 4-chloro-2-methylaniline (68 mg, 0.48 mmol), Pd₂dba₃ (18 mg, 20 μmol), XPhos (38 mg, 80 μmol) and Cs₂CO₃ (287 mg, 0.88 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (80-100% EtOAc/pet. ether) to give imidazopyridinone 122 (111 mg, 74%) as grey crystals: mp (EtOAc/pet ether) 203-205° C.; ¹H NMR (CDCl₃) δ 7.83 (s, 1H, H-4), 7.43 (d, J=8.5 Hz, 1H, H-6″), 7.22 (d, J=2.5 Hz, 1H, H-3″), 7.17 (dd, J=8.5, 2.5 Hz, 1H, H-5″), 6.52 (d, J=0.6 Hz, 1H, H-7), 6.02 (s, 1H, 6-NH), 4.48 (tt, J=12.4, 4.3 Hz, 1H, H-4′), 4.10 (dd, J=11.8, 4.4 Hz, 2 H, H-2′, H-6′), 3.52 (dt, J=12.0, 1.7 Hz, 2 H, H-2′, H-6′), 3.40 (s, 3H, 3-CH₃), 2.33 (dq, J=12.7, 4.7 Hz, 2 H, H-3′, H-5′), 2.27 (s, 3H, 2″-CH₃), 1.73 (dd, J=12.5, 2.5 Hz, 2 H, H-3′, H-5′); MS m/z 373.2 (MH⁺, 100%). Anal calcd for C₁₉H21ClN₄O₂: C, 61.21; H, 5.68; N, 15.03. Found: C, 61.24; H, 5.84; N, 15.01%. HPLC purity 98.4%.

### Example 96: SN39538 3-Methyl-6-((2-methyl-5-(methylsulfonyl)phenyl)amino)-1-(tetrahydro-2H-pyran-4-yl)-1,3-dihydro-2H-imidazo[4,5-c]pyridin-2-one (123)

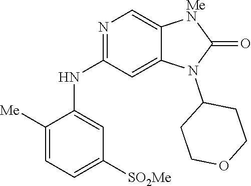 [SMILES: Cc1ccc(S(C)(=O)=O)cc1Nc1cc2c(cn1)n(C)c(=O)n2C1CCOCC1]

A degassed mixture of chloride 120 (104 mg, 0.39 mmol), 2-methyl-5-(methylsulfonyl)aniline (86 mg, 0.47 mmol), Pd₂dba₃ (18 mg, 20 μmol), XPhos (37 mg, 78 μmol) and Cs₂CO₃ (280 mg, 0.86 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (EtOAc/pet. ether) to give imidazopyridinone 123 (8 mg, 5%) as a tan powder: mp (EtOAc/pet ether) 302-305° C.; ¹H NMR (CDCl₃) δ 8.23 (d, J=1.8 Hz, 1H, H-6″), 7.90 (s, 1H, H-4), 7.51 (dd, J=7.9, 1.8 Hz, 1H, H-5″), 7.39 (d, J=7.9 Hz, 1H, H-3″), 6.83 (s, 1H, H-7), 6.30 (s, 1H, 6-NH), 4.58 (tt, J=12.5, 4.4 Hz, 1H, H-4′), 4.11 (dd, J=11.6, 4.4 Hz, 2 H, H-2′, H-6′), 3.53 (dt, J=12.0, 1.6 Hz, 2 H, H-2′, H-6′), 3.44 (s, 3H, 5″-SO₂CH₃), 3.04 (s, 3H, 3-CH₃), 2.31-2.40 (m, 5H, H-3′, H-5′, 2″-CH₃), 1.79 (dd, J=12.6, 2.8 Hz, 2 H, H-3′, H-5′); MS m/z 417.2 (MH⁺, 100%). HRMS calcd for C₂₀H25N₄O₄S (MH⁺) m/z 417.1591, found 417.1589 (0.5 ppm). HPLC purity 89.0%.

### Example 97: SN39871 6-((4-(Benzyloxy)-2-methylphenyl)amino)-3-methyl-1-(tetrahydro-2H-pyran-4-yl)-1,3-dihydro-2H-imidazo[4,5-c]pyridin-2-one (124)

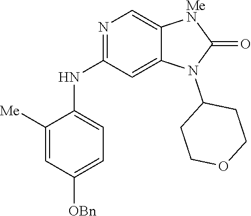 [SMILES: Cc1cc(OCc2ccccc2)ccc1Nc1cc2c(cn1)n(C)c(=O)n2C1CCOCC1]

A degassed mixture of chloride 120 (415 mg, 1.55 mmol), 4-(benzyloxy)-2-methylaniline (397 15 mg, 1.86 mmol), Pd₂dba₃ (71 mg, 78 μmol), XPhos (148 mg, 310 μmol) and Cs₂CO₃ (1.10 g, 3.41 mmol) in MeCN (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 124 (538 mg, 78%) as white needles: mp (EtOAc/pet ether) 179-181° C.; ¹H NMR (CDCl₃) δ 7.78 (s, 1H, H-4), 7.47 (br d, J=7.0 Hz, 2 H, H-2″′, H-6″′), 7.40 (br dd, J=7.5, 7.1 Hz, 2 H, H-3″′, H-5″′), 7.34 (br t, J=7.2 Hz, 1H, H-4″′), 7.26 (d, J=8.6 Hz, 1H, H-6″), 6.93 (d, J=2.9 Hz, 1H, H-3″), 6.84 (dd, J=8.6, 2.9 Hz, 1H, H-5″), 6.26 (d, J=0.5 Hz, 1H, H-7), 5.94 (s, 1H, 6-NH), 5.08 (s, 2 H CH₂O), 4.41 (tt, J=12.4, 4.2 Hz, 1 H, H-4′), 4.07 (dd, J=11.6, 4.4 Hz, 2 H, H-2′, H-6′), 3.50 (dt, J=11.9, 1.6 Hz, 2 H, H-2′, H-6′), 3.33 (s, 3H, 3-CH₃), 2.20-2.35 (m, 5H, 2″-CH₃, H-3′, H-5′), 1.69 (dd, J=12.4, 2.5 Hz, 2 H, H-3′, H-5′); MS m/z 445.2 (MH⁺, 100%); HRMS calcd for C₂₆H₂₉N₄O₃ (MH⁺) m/z 445.2234, found 445.2250 (−3.6 ppm). HPLC purity 98.9%.

### Example 98: SN40019 3-Methyl-6-((7-methyl-[1,2,4]triazolo[1,5-a]pyridin-6-yl)amino)-1-(tetrahydro-2H-pyran-4-yl)-1,3-dihydro-2H-imidazo[4,5-c]pyridin-2-one (125)

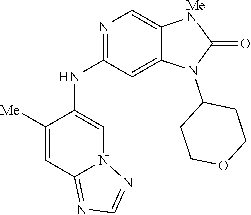 [SMILES: Cc1cc2ncnn2cc1Nc1cc2c(cn1)n(C)c(=O)n2C1CCOCC1]

A degassed mixture of chloride 120 (133 mg, 0.50 mmol), 7-methyl-[1,2,4]triazolo[1,5-a]pyridin-6-amine (74 mg, 0.50 mmol), BrettPhos G3 (27 mg, 50 μmol) and Cs₂CO₃ (358 mg, 1.10 mmol) in dioxane (8 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 125 (88 mg, 46%) as a tan solid: mp 260-263° C.; ¹H NMR (CDCl₃) δ 9.40 (s, 1H, H-5″), 8.26 (s, 1H, H-2″), 7.87 (s, 1H, H-4), 7.59 (s, 1H, H-8″), 6.53 (s, 1H, H-7), 6.10 (s, 1H, 6-NH), 4.53 (tt, J=12.4, 4.4 Hz, 1 H, H-4′), 4.12 (dd, J=11.7, 4.5 Hz, 2 H, H-2′, H-6′), 3.55 (dt, J=12.0, 1.7 Hz, 2 H, H-2′, H-6′), 3.43 (s, 3H, 3-CH₃), 2.47 (s, 3H, 7″-CH₃), 2.39 (dq, J=12.6, 4.7 Hz, 2 H, H-3′, H-5′), 1.77 (dd, J=12.5, 2.8 Hz, 2 H, H-3′, H-5′); MS m/z 380.2 (MH⁺, 100%); HRMS calcd for C₁₉H₂₂N₇₀₂ (MH⁺) m/z 380.1829, found 380.1833 (−0.9 ppm). HPLC purity 97.3%.

### Example 99: SN40037 6-((6-Methoxy-4-methylpyridin-3-yl)amino)-3-methyl-1-(tetrahydro-2H-pyran-4-yl)-1,3-dihydro-2H-imidazo[4,5-c]pyridin-2-one (126)

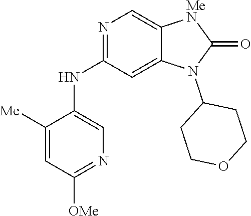 [SMILES: COc1cc(C)c(Nc2cc3c(cn2)n(C)c(=O)n3C2CCOCC2)cn1]

A degassed mixture of chloride 120 (130 mg, 0.49 mmol), 6-methoxy-4-methylpyridin-3-amine (67 mg, 0.49 mmol), BrettPhos G3 (26 mg, 49 μmol) and Cs₂CO₃ (335 mg, 1.03 mmol) in dioxane (6 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 126 (132 mg, 73%) as a tan powder: ¹H NMR (CDCl₃) δ 8.14 (s, 1H, H-2″), 7.77 (s, 1H, H-4), 6.69 (s, 1H, H-5″), 6.20 (d, J=0.5 Hz, 1H, H-7), 5.92 (br s, 1H, 6-NH), 4.40 (tt, J=12.4, 4.3 Hz, 1H, H-4′), 4.08 (dd, J=11.6, 4.5 Hz, 2 H, H-2′, H-6′), 3.95 (s, 3H, 6″-OCH₃), 3.50 (dt, J=12.0, 1.7 Hz, 2 H, H-2′, H-6′), 3.38 (s, 3H, 3-CH₃), 2.30 (dq, J=12.6, 4.6 Hz, 2 H, H-3′, H-5′), 2.22 (s, 3H, 4″-CH₃), 1.68 (dd, J=12.5, 2.5 Hz, 2 H, H-3′, H-5′); MS m/z 370.2 (MH⁺, 100%); HRMS calcd for C₁₉H₂₄N₅O₃(MH⁺) m/z 370.1874, found 370.1877 (−0.8 ppm). HPLC purity 98.1%.

### Example 100: SN40046 6-((2,5-Dimethylbenzo[d]thiazol-6-yl)amino)-3-methyl-1-(tetrahydro-2H-pyran-4-yl)-1,3-dihydro-2H-imidazo[4,5-c]pyridin-2-one (127)

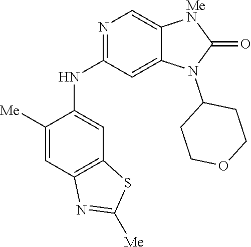 [SMILES: Cc1nc2cc(C)c(Nc3cc4c(cn3)n(C)c(=O)n4C3CCOCC3)cc2s1]

2,5-Dimethyl-6-nitrobenzo[d]thiazole. A mixture of KNO₃ (1.36 g, 13.5 mmol) and 2,5-dimethylbenzo[d]thiazole (2.0 g, 12.3 mmol) was added in portions to stirred cH₂SO₄ (25 mL) at −5° C. The mixture was allowed to warm to 20° C. over 4 and then poured onto ice (300 mL).

The mixture was stirred for 10 min, then filtered and the solid dried. The residue was purified by chromatography, eluting with a gradient (10-20%) of EtOAc/pet. ether, to give: a) 2,5-dimethyl-4-nitrobenzo[d]thiazole (1.19 g, 95%) as white plates: mp (EtOAc/pet ether) 126-128° C.; ¹H NMR (CD₃Cl) δ 8.72 (d, J=8.2 Hz, 1H, H-7), 7.29 (d, J=8.2 Hz, 1H, H-6), 2.87 (s, 3H, 2-CH₃), 2.50 (s, 3H, 5-CH₃); MS m/z 208.1 (MH⁺, 100%); and b) 2,5-dimethyl-6-nitrobenzo[d]thiazole (1.22 g, 48%) as a cream powder: mp (EtOAc/pet ether) 152-154° C.; 1H NMR (CD₃Cl) δ 8.53 (s, 1H, H-7), 7.86 (s, 1H, H-4), 2.89 (s, 3H, 2-CH₃), 2.73 (s, 3H, 5-CH₃); MS m/z 208.1 (MH⁺, 100%). 2,5-Dimethylbenzo[d]thiazol-6-amine. A solution of 2,5-dimethyl-6-nitrobenzo[d]thiazole (0.73 g, 3.51 mmol) in EtOAc (30 ml) was added dropwise to a stirred suspension of SnCl₂·2H₂O (3.16 g, 14.0 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated. The residue was purified by chromatography, eluting with a gradient (30-50%) of EtOAc/pet. ether, to give amine (0.31 g, 50%) as a white powder: mp 167-170° C.; ¹H NMR (CD₃Cl) δ 7.62 (s, 1H, H-4), 7.03 (s, 1H, H-7), 3.71 (br s, 2H, 6-NH₂), 2.75 (s, 3H, 2-CH₃), 2.28 (s, 3H, 5-CH₃); MS m/z 179.1 (MH⁺, 100%).

6-((2,5-Dimethylbenzo[d]thiazol-6-yl)amino)-3-methyl-1-(tetrahydro-2H-pyran-4-yl)-1,3-dihydro-2H-imidazo[4,5-c]pyridin-2-one (127). A degassed mixture of chloride 120 (120 mg, 0.45 mmol), 2,5-dimethylbenzo[d]thiazol-6-amine (80 mg, 0.45 mmol), BrettPhos G3 (24 mg, 46 μmol) and Cs₂CO₃ (308 mg, 0.95 mmol) in dioxane (6 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 127 (125 mg, 68%) as a tan powder: mp (EtOAc/pet ether) 288-291° C.; ¹H NMR [(CD₃)₂SO] δ 8.57 (br s, 1H, 6-NH), 7.93 (s, 1H, H-7), 7.90 (s, 1H, H-4), 7.70 (s, 1H, H-4″), 7.08 (s, 1H, H-7), 4.41 (tt, J=12.3, 4.1 Hz, 1H, H-4′), 4.00 (dd, J=11.4, 4.1 Hz, 2 H, H-2′, H-6′), 3.48 (br t, J=11.7, Hz, 2 H, H-2′, H-6′), 3.30 (s, 3H, 3-CH₃), 2.73 (s, 3H, 2″-CH₃), 2.39 (s, 3H, 5″-CH₃), 2.28 (dq, J=12.5, 4.5 Hz, 2 H, H-3′, H-5′), 1.68 (br dd, J=12.3, 2.8 Hz, 2 H, H-3′, H-5′); MS m/z 410.2 (MH⁺, 100%); HRMS calcd for C₂₁H₂₄N₅O₂S (MH⁺) m/z 410.1645, found 410.1655 (−2.3 ppm). HPLC purity 97.0%.

### Example 101: SN40050 3-Methyl-6-((2-methylbenzo[d]oxazol-6-yl)amino)-1-(tetrahydro-2H-pyran-4-yl)-1,3-dihydro-2H-imidazo[4,5-c]pyridin-2-one (128)

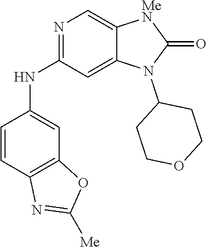 [SMILES: Cc1nc2ccc(Nc3cc4c(cn3)n(C)c(=O)n4C3CCOCC3)cc2o1]

A degassed mixture of chloride 120 (120 mg, 0.45 mmol), 2-methylbenzo[d]oxazol-6-amine (64 mg, 0.43 mmol), BrettPhos G3 (24 mg, 46 μmol) and Cs₂CO₃ (296 mg, 0.91 mmol) in dioxane (6 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 128 (126 mg, 77%) as a white powder: mp (EtOAc/pet ether) 274-276° C.; ¹H NMR [(CD₃)₂SO] δ 9.04 (br s, 1H, 6-NH), 8.35 (d, J=1.8 Hz, 1H, H-7), 7.98 (s, 1H, H-4), 7.46 (d, J=8.6 Hz, 1H, H-4″), 7.24 (dd, J=8.6, 1.9 Hz, 1 H, H-5″), 6.84 (s, 1H, H-7), 4.42 (tt, J=12.3, 4.2 Hz, 1H, H-4′), 4.00 (dd, J=11.3, 4.1 Hz, 2 H, H-2′, H-6′), 3.48 (br dd, J=11.5, 11.1 Hz, 2 H, H-2′, H-6′), 3.31 (s, 3H, 3-CH₃), 2.55 (s, 3H, 2″-CH₃), 2.26 (dq, J=12.5, 4.5 Hz, 2 H, H-3′, H-5′), 1.69 (br dd, J=12.5, 2.7 Hz, 2 H, H-3′, H-5′); MS m/z 380.2 (MH⁺, 100%); HRMS calcd for C₂₀H₂₂N₅O₃(MH⁺) m/z 380.1717, found 380.1721 (−0.9 ppm). HPLC purity 98.4%.

### Example 102: SN40070 Ethyl 7-Methyl-6-((3-methyl-2-oxo-1-(tetrahydro-2H-pyran-4-yl)-2,3-dihydro-1H-imidazo[4,5-c]pyridin-6-yl)amino)imidazo[1,2-a]pyridine-3-carboxylate (129)

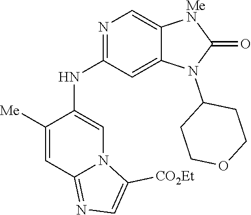 [SMILES: CCOC(=O)c1cnc2cc(C)c(Nc3cc4c(cn3)n(C)c(=O)n4C3CCOCC3)cn12]

Ethyl 7-Methyl-6-nitroimidazo[1,2-a]pyridine-2-carboxylate. Ethyl bromopyruvate (1.90 g, 9.80 mmol) in dioxane (2 mL) was added to a stirred suspension of 4-methyl-5-nitropyridin-2-amine (1.0 g, 6.53 mmol) and NaHCO₃(1.1 g, 13.1 mmol) in dioxane (25 mL) at 20° C. The mixture was stirred at 100° C. for 16 h. The solvent was evaporated. The residue was suspended in water (100 mL) filtered and washed with pet. ether and dried to give ester (1.10 g, 68%) as a tan powder: mp (water) 160-162° C.; ¹H NMR [(CD₃)₂SO] δ 9.71 (s, 1H, H-5), 8.64 (d, J=0.6 Hz, 1H, H-3), 7.67 (d, J=0.9 Hz, 1H, H-8), 4.33 (q, J=7.1 Hz, 2 H, CH₂O), 2.39 (d, J=0.9 Hz, 3 H, 7-CH₃), 1.33 (t, J=7.1 Hz, 3 H, CH₃); MS m/z 250.1 (MH⁺, 100%); HRMS calcd for C₁₁H₁₂N₃04 (MH⁺) m/z 250.0822, found 250.0818 (1.8 ppm).

Ethyl 6-Amino-7-methylimidazo[1,2-a]pyridine-3-carboxylate. A mixture of nitropyridine (1.00 g, 4.01 mmol) Pd/C (100 mg) and NH₄HCO₂ (1.26 g, 20.1 mmol) in EtOH (50 ml) was stirred at 80° C. The mixture was cooled to 20° C. and filtered through a pad of diatomaceous earth and the pad was washed with EtOH (20 mL). The solvent was evaporated. The residue was purified by chromatography, eluting with a gradient (70-100%) of EtOAc/pet. ether, to give amine (0.22 g, 25%) as a tan solid: ¹H NMR (CD₃Cl) δ 8.90 (s, 1H, H-5), 8.00 (s, 1H, H-3), 7.44 (s, 1H, H-8), 5.14 (br s, 2H, 6-NH₂), 4.30 (q, J=7.1 Hz, 2 H, CH₂O), 2.24 (d, J=0.7 Hz, 3 H, 7-CH₃), 1.32 (t, J=7.1 Hz, 3 H, CH₃); MS m/z 220.1 (MH⁺, 100%); HRMS calcd for C₁₁H₁₄N₃O₂ (MH⁺) m/z 220.1081, found 220.1074 (3.0 ppm).

Ethyl 7-Methyl-6-((3-methyl-2-oxo-1-(tetrahydro-2H-pyran-4-yl)-2,3-dihydro-1H-imidazo[4,5-c]pyridin-6-yl)amino)imidazo[1,2-a]pyridine-3-carboxylate (129). A degassed mixture of chloride 120 (239 mg, 0.89 mmol), ethyl 6-amino-7-methylimidazo[1,2-a]pyridine-3-carboxylate (196 mg, 0.89 mmol), BrettPhos G3 (48 mg, 89 μmol) and Cs₂CO₃ (640 mg, 1.87 mmol) in dioxane (8 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 129 (264 mg, 66%) as a tan powder: mp (EtOAc/pet ether) 240-243° C.; ¹H NMR [(CD₃)₂SO] δ 10.15 (br s, 1 H, 6′-NH), 8.14 (s, 1H, H-5), 7.98 (s, 1H, H-3), 7.97 (s, 1H, H-4′), 7.63 (s, 1H, H-8), 7.17 (s, 1H, H-7), 4.43 (tt, J=12.3, 4.1 Hz, 1H, H-4″), 4.32 (q, J=7.1 Hz, 2 H, CH₂O), 4.02 (dd, J=11.2, 4.0 Hz, 2 H, H-2″, H-6″), 3.49 (br dd, J=11.7, 10.7 Hz, 2 H, H-2″, H-6″), 3.35 (s, 3H, 3′-CH₃), 2.31 (dq, J=12.4, 4.4 Hz, 2 H, H-3″, H-5″), 2.44 (d, J=0.5 Hz, 3 H, 7-CH₃), 1.61 (ddd, J=12.3, 11.8, 2.8 Hz, 2 H, H-3″, H-5″), 1.33 (t, J=7.1 Hz, 3 H, CH₃); MS m/z 410.2 (MH⁺, 100%); HRMS calcd for C₂₃H₂₆N₆O₄ (MH⁺) m/z 451.2088, found 451.2087 (0.3 ppm). HPLC purity 97.4%.

### Example 104: SN40092 3-Methyl-6-((6-methylbenzo[d][1,3]dioxol-5-yl)amino)-1-(tetrahydro-2H-pyran-4-yl)-1,3-dihydro-2H-imidazo[4,5-c]pyridin-2-one (130)

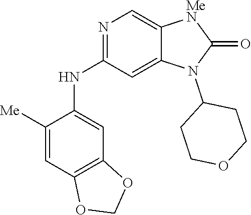 [SMILES: Cc1cc2c(cc1Nc1cc3c(cn1)n(C)c(=O)n3C1CCOCC1)OCO2]

5-Methyl-6-nitrobenzo[d][1,3]dioxole. 70% HNO₃ (2.2 mL) was added dropwise to a stirred solution of 5-methylbenzo[d][1,3]dioxole (2.15 g, 15.8 mmol) in HOAc (10 mL) at 10° C. and the mixture was stirred at 20° C. for 1 h. then diluted with ice/water (100 mL). The mixture was stirred for 30 min, then filtered and the solid washed with water (5 mL) and dried to give nitrobenzodioxole (2.72 g, 95%) as a white powder: mp (water) 81-82° C.; ¹H NMR (CD₃Cl) δ 7.55 (s, 1H, H-7), 6.72 (s, 1H, H-4), 6.08 (s, 2H, H-2), 2.57 (s, 3H, 5-CH₃); MS m/z 182.1 (MH⁺, 100%).

6-Methylbenzo[d][1,3]dioxol-5-amine. A solution of 5-methyl-6-nitrobenzo[d][1,3]dioxole (0.98 g, 5.38 mmol) in EtOH (50 ml) was stirred vigorously with Pd/C (50 mg) under Hz (50 psi) for 6 h. The mixture was filtered through a pad of diatomaceous earth and the pad washed with EtOH (10 mL). The filtrate was evaporated to give 6-methylbenzo[d][1,3]dioxol-5-amine (0.82 g, 100%) as a tan powder: mp 83-85° C.; ¹H NMR (CD₃Cl) δ 6.56 (s, 1H, H-7), 6.29 (s, 1H, H-4), 5.82 (s, 2H, H-2), 3.77 (br s, 2H, 5-NH₂), 2.09 (s, 3H, 6-CH₃); MS m/z 152.1 (MH⁺, 100%). 3-Methyl-6-((6-methylbenzo[d][1,3]dioxol-5-yl)amino)-1-(tetrahydro-2H-pyran-4-yl)-1,3-dihydro-2H-imidazo[4,5-c]pyridin-2-one (130). A degassed mixture of chloride 120 (126 mg, 0.47 mmol), 6-methylbenzo[d][1,3]dioxol-5-amine (71 mg, 0.47 mmol), BrettPhos G3 (25 mg, 47 μmol) and Cs₂CO₃ (322 mg, 0.99 mmol) in dioxane (6 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated.

The residue was purified by chromatography, eluting with EtOAc, to give imidazopyridinone 130 (126 mg, 70%) as a tan powder: mp (EtOAc/pet ether) 186-188° C.; ¹H NMR (CD₃Cl) δ 7.78 (s, 1H, H-4), 6.89 (s, 1H, H-7), 6.75 (s, 1H, H-4″), 6.26 (s, 1H, H-7), 5.96 (s, 1H, H-2″), 5.90 (br s, 1H, 6-NH), 4.44 (tt, J=12.4, 4.2 Hz, 1H, H-4′), 4.10 (dd, J=11.5, 4.6 Hz, 2 H, H-2′, H-6′), 3.51 (br dt, J=11.7, Hz, 1.7 Hz, 2 H, H-2′, H-6′), 3.39 (s, 3H, 3-CH₃), 2.30 (dq, J=12.6, 4.6 Hz, 2 H, H-3′, H-5′), 2.19 (s, 3H, 6′-CH₃), 1.71 (br dd, J=12.4, 2.5 Hz, 2 H, H-3′, H-5′); MS m/z 383.2 (MH⁺, 100%); HRMS calcd for C₂₀H₂₃N₄O₄ (MH⁺) m/z 383.1714, found 383.1721 (−1.9 ppm). HPLC purity 99.3%.

### Example 105: SN40151 6-((2,6-Dimethylbenzo[d]oxazol-5-yl)amino)-3-methyl-1-(tetrahydro-2H-pyran-4-yl)-1,3-dihydro-2H-imidazo[4,5-c]pyridin-2-one (131)

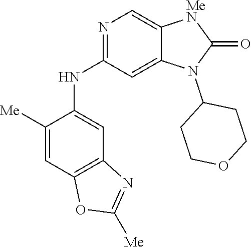 [SMILES: Cc1nc2cc(Nc3cc4c(cn3)n(C)c(=O)n4C3CCOCC3)c(C)cc2o1]

2-Amino-5-methylphenol. A mixture of 5-methyl-2-nitrophenol (4.36 g, 28.5 mmol) and Pd/C (200 mg) in EtOH (100 ml) was stirred vigorously with under H2 (50 psi) for 2 h. The mixture was filtered through a pad of diatomaceous earth and the pad washed with EtOH (50 mL). The filtrate was evaporated to give amine (3.48 g, 99%) as a yellow solid: mp 154-157° C.; ¹H NMR (CD₃Cl) δ 8.80 (br s, 1H, OH), 6.44 (m, 2H, H-3, H-6), 6.34 (ddd, J=7.8, 1.9, 0.7 Hz, 1H, H-4), 4.24 (br s, 2H, 2-NH₂), 2.08 (s, 3H, 5-CH₃); MS m/z 124.1 (MH⁺, 100%). 2,6-Dimethylbenzo[d]oxazole. Ac₂O (12.9 mL, 136.4 mmol) was added to a stirred solution of phenol (3.36 g, 27.3 mmol) and p-toluenesulfonic acid (0.52 g, 2.7 mmol) in dry toluene and the mixture was stirred at 60° C. for 1 h. More p-toluenesulfonic acid (5.71 g, 30.0 mmol) was added and the mixture stirred at 120° C. for 2 h. The reaction was cooled to 20° C. and quenched with ice/water (150 mL), diluted with EtOAc (100 mL) and the mixture washed with water (2×50 mL), washed with brine (50 mL) and dried (MgSO₄). The solvent was evaporated and the residue was purified by chromatography, eluting with 30% EtOAc/pet. ether, to give benzoxazole (3.50 g, 87%) as a clear oil: ¹H NMR (CD₃Cl) δ 7.51 (d, J=8.1 Hz, 1H, H-4), 7.27 (t, J=0.7 Hz, 1H, H-7), 7.10 (ddd, J=8.1, 1.5, 0.6 Hz, 1H, H-5), 2.61 (s, 3H, 2-CH₃), 2.47 (s, 3H, 6-CH₃); MS m/z 148.1 (MH⁺, 100%).

2,6-Dimethyl-5-nitrobenzo[d]oxazole. 70% HNO₃ (1.8 mL, 23.4 mmol) was added dropwise to a stirred solution of 2,6-dimethylbenzo[d]oxazole (3.50 g, 23.4 mmol) in cH₂SO₄ (20 mL) at 5° C. and the mixture was stirred at 20° C. for 1 h. The reaction was poured into ice/water (100 mL) and the mixture was stirred for 30 min, then filtered and the solid washed with water (50 mL) and dried. The solid was purified by chromatography, eluting with 30% EtOAc/pet. ether, to give nitrobenzoxazole (3.24 g, 72%) as a white powder: mp 137-139° C.; ¹H NMR (CD₃Cl) δ 8.30 (s, 1H, H-4), 7.41 (s, 1H, H-7), 2.70 (s, 3H, 2-CH₃), 2.68 (s, 3H, 6-CH₃); MS m/z 193.1 (MH⁺, 100%).

2,6-Dimethylbenzo[d]oxazol-5-amine. A mixture of 2,6-dimethyl-5-nitrobenzo[d]oxazole (0.92 g, 4.79 mmol), NH₄HCO₂ (1.51 g, 23.9 mmol) and Pd/C (100 mg) in EtOH (50 ml) was stirred vigorously at 85° C. for 2 h. The mixture was cooled to 20° C. and filtered through a pad of diatomaceous earth and the pad washed with EtOH (10 mL). The filtrate was evaporated and the residue was purified by chromatography, eluting with a gradient (0-10%) of MeOH/EtOAc, to give 2,6-dimethylbenzo[d]oxazol-5-amine (0.77 g, 100%) as a grey powder: mp 120-121° C.; ¹H NMR (CD₃Cl) δ 7.16 (s, 1H, H-7), 6.93 (s, 1H, H-4), 3.49 (br s, 2H, 5-NH₂), 2.51 (s, 3H, 2-CH₃), 2.62 (s, 3H, 6-CH₃); MS m/z 163.1 (MH⁺, 100%).

6-((2,6-Dimethylbenzo[d]oxazol-5-yl)amino)-3-methyl-1-(tetrahydro-2H-pyran-4-yl)-1,3-dihydro-2H-imidazo[4,5-c]pyridin-2-one (131). A degassed mixture of chloride 120 (103 mg, 0.39 mmol), 2,6-dimethylbenzo[d]oxazol-5-amine (62 mg, 0.39 mmol), BrettPhos G3 (21 mg, 39 μmol) and Cs₂CO₃ (267 mg, 0.82 mmol) in dioxane (6 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 131 (82 mg, 53%) as a tan powder: mp (EtOAc/pet ether) 250-252° C.; ¹H NMR (CD₃Cl) δ 7.81 (s, 1H, H-4), 7.66 (s, 1H, H-7), 7.38 (s, 1H, H-4″), 6.39 (s, 1H, H-7), 6.17 (br s, 1H, 6-NH), 4.51 (tt, J=12.5, 4.2 Hz, 1H, H-4′), 4.05 (dd, J=11.6, 4.2 Hz, 2 H, H-2′, H-6′), 3.46 (br dt, J=12.1, 1.6 Hz, 2 H, H-2′, H-6′), 3.40 (s, 3H, 3-CH₃), 2.63 (s, 3H, 2″-CH₃), 2.38 (s, 3H, 6″-CH₃), 2.27 (dq, J=12.6, 4.6 Hz, 2 H, H-3′, H-5′), 1.70 (br dd, J=12.5, 2.5 Hz, 2 H, H-3′, H-5′); MS m/z 394.2 (MH⁺, 100%); HRMS calcd for C₂₁H₂₄N₅O₃(MH⁺) m/z 394.1874, found 394.1874 (−0.1 ppm). HPLC purity 98.0%.

### Example 106: SN40152 6-((2,5-Dimethylbenzo[d]oxazol-6-yl)amino)-3-methyl-1-(tetrahydro-2H-pyran-4-yl)-1,3-dihydro-2H-imidazo[4,5-c]pyridin-2-one (132)

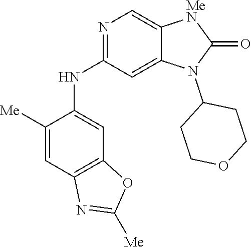 [SMILES: Cc1nc2cc(C)c(Nc3cc4c(cn3)n(C)c(=O)n4C3CCOCC3)cc2o1]

2-Amino-4-methylphenol. A mixture of 4-methyl-2-nitrophenol (3.24 g, 21.2 mmol) and Pd/C (200 mg) in EtOH (100 ml) was stirred vigorously with under H2 (50 psi) for 2 h. The mixture was filtered through a pad of diatomaceous earth and the pad washed with EtOH (50 mL). The filtrate was evaporated to give amine (2.59 g, 99%) as a tan solid: MS m/z 124.1 (MH⁺, 100%).

2,5-Dimethylbenzo[d]oxazole. Ac₂O (9.9 mL, 105.0 mmol) was added to a stirred solution of 2-amino-4-methylphenol (2.59 g, 21.0 mmol) and p-toluenesulfonic acid (0.40 g, 2.1 mmol) in dry toluene and the mixture was stirred at 60° C. for 1 h. More p-toluenesulfonic acid (4.40 g, 23.1 mmol) was added and the mixture stirred at 120° C. for 2 h. The reaction was cooled to 20° C. and quenched with ice/water (150 mL), diluted with EtOAc (100 mL) and the mixture washed with water (2×50 mL), washed with brine (50 mL) and dried (MgSO₄). The solvent was evaporated and the residue was purified by chromatography, eluting with 50% EtOAc/pet. ether, to give benzoxazole (3.09 g, 100%) as a clear oil: ¹H NMR (CD₃Cl) δ 7.43 (s, 1H, H-4), 7.33 (d, J=8.3 Hz, 1H, H-7), 7.09 (dd, J=8.3, 1.1 Hz, 1H, H-6), 2.62 (s, 3H, 2-CH₃), 2.45 (s, 3H, 5-CH₃); MS m/z 148.1 (MH⁺, 100%).

2,5-Dimethyl-6-nitrobenzo[d]oxazole. 70% HNO₃ (1.5 mL, 21.0 mmol) was added dropwise to a stirred solution of 2,5-dimethylbenzo[d]oxazole (3.09 g, 21.0 mmol) in cH₂SO₄ (20 mL) at 5° C. and the mixture was stirred at 20° C. for 1 h. The reaction was poured into ice/water (100 mL) and the mixture was stirred for 30 min, then filtered and the solid washed with water (50 mL) and dried. The solid was purified by chromatography, eluting with 30% EtOAc/pet. ether, to give nitrobenzoxazole (3.09 g, 76%) as a cream powder: mp 130-132° C.; ¹H NMR (CD₃Cl) δ 8.18 (s, 1H, H-7), 7.57 (s, 1H, H-4), 2.70 (s, 3H, 2-CH₃), 2.69 (s, 3H, 5-CH₃); MS m/z 193.1 (MH⁺, 100%).

2,5-Dimethylbenzo[d]oxazol-6-amine. A mixture of 2,5-dimethyl-6-nitrobenzo[d]oxazole (0.99 g, 5.15 mmol), NH₄HCO₂ (1.62 g, 25.6 mmol) and Pd/C (100 mg) in EtOH (50 ml) was stirred vigorously at 85° C. for 2 h. The mixture was cooled to 20° C. and filtered through a pad of diatomaceous earth and the pad washed with EtOH (10 mL). The filtrate was evaporated and the residue was purified by chromatography, eluting with a gradient (0-10%) of MeOH/EtOAc, to give 2,5-dimethylbenzo[d]oxazol-6-amine (0.77 g, 100%) as a grey powder: mp 120-121° C.; ¹H NMR (CD₃Cl) δ 7.30 (s, 1H, H-4), 6.78 (s, 1H, H-7), 3.68 (br s, 2H, 6-NH₂), 2.55 (s, 3H, 2-CH₃), 2.24 (s, 3H, 5-CH₃); MS m/z 163.1 (MH⁺, 100%).

6-((2,5-Dimethylbenzo[d]oxazol-6-yl)amino)-3-methyl-1-(tetrahydro-2H-pyran-4-yl)-1,3-dihydro-2H-imidazo[4,5-c]pyridin-2-one (132). A degassed mixture of chloride 120 (38 mg, 0.14 mmol), 2,5-dimethylbenzo[d]oxazol-6-amine (25 mg, 0.16 mmol), BrettPhos G3 (8 mg, 14 μmol) and Cs₂CO₃ (97 mg, 0.30 mmol) in dioxane (6 mL) was stirred in a sealed tube at 120° C. for 4 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (0-10%) of MeOH/EtOAc, to give imidazopyridinone 132 (15 mg, 27%) as a tan powder: mp (EtOAc/pet ether) 243-245° C.; ¹H NMR (CD₃Cl) δ 7.85 (s, 1H, H-4), 7.70 (s, 1H, H-4″), 7.50 (s, 1H, H-7), 6.52 (d, J=0.5 Hz, 1H, H-7), 6.17 (br s, 1H, 6-NH), 4.48 (tt, J=12.4, 4.3 Hz, 1H, H-4′), 4.09 (dd, J=11.8, 4.4 Hz, 2 H, H-2′, H-6′), 3.52 (br dt, J=12.0, 1.6 Hz, 2 H, H-2′, H-6′), 3.42 (s, 3H, 3-CH₃), 2.62 (s, 3H, 2″-CH₃), 2.38 (s, 3H, 5″-CH₃), 2.32 (dq, J=12.5, 4.6 Hz, 2 H, H-3′, H-5′), 1.74 (br dd, J=12.5, 2.5 Hz, 2 H, H-3′, H-5′); MS m/z 394.2 (MH⁺, 100%); HRMS calcd for C₂₁H₂₄N₅O₃(MH⁺) m/z 394.1874, found 394.1870 (0.1 ppm). HPLC purity 98.9%.

### Example 107: SN39689 Benzyl (4-Methoxy-2-methylphenyl)(3-methyl-2-oxo-1-(tetrahydro-2H-pyran-4-yl)-2,3-dihydro-1H-imidazo[4,5-c]pyridin-6-yl)carbamate (133)

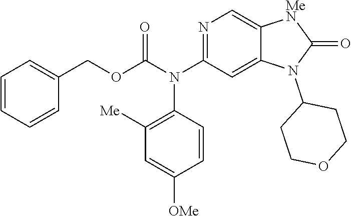 [SMILES: COc1ccc(N(C(=O)OCc2ccccc2)c2cc3c(cn2)n(C)c(=O)n3C2CCOCC2)c(C)c1]

A solution of benzyl chloroformate (19 μL, 0.13 mmol) in dry THF (2 mL) was added to a stirred solution of imidazopyridinone 121 (41 mg, 0.11 mmol) and iPr₂NEt (27 μL, 150 μmol) in dry THF (5 mL) and the mixture at 20° C. for 16 h. The solvent was evaporated and the residue was partitioned between EtOAc (20 ml) and water (20 mL). The organic fraction was washed with water (20 mL), washed with brine (10 mL), dried (MgSO₄), filtered and the solvent evaporated. The residue was purified by chromatography, eluting with 70% EtOAc/pet. ether, to give carbamate 133 (52 mg, 93%) as a clear gum: ¹H NMR (CDCl₃) δ 7.93 (s, 1H, H-4′), 7.55 (s, 1H, H-7), 7.22-7.33 (m, 5H, H-2″″, H-3″″, H-4″″, H-5″″, H-6), 7.13 (d, J=8.5 Hz, 1 H, H-6″′), 6.79 (d, J=2.9 Hz, 1H, H-3″′), 6.75 (dd, J=8.5, 2.9 Hz, 1H, H-5″′), 5.22 (s, 2H, CH₂O), 4.51 (tt, J=12.4, 4.2 Hz, 1H, H-4″), 4.12 (dd, J=11.6, 4.2 Hz, 2 H, H-2″, H-6″), 3.80 (s, 3H, 4″′—OCH₃), 3.56 (dt, J=11.9, 1.5 Hz, 2 H, H-2″, H-6″), 3.38 (s, 3H, 3′-CH₃), 2.42 (dq, J=12.5, 4.6 Hz, 2 H, H-3″, H-5″), 2.17 (s, 3H, 2″′-CH₃), 1.75 (dd, J=12.5, 2.6 Hz, 2 H, H-3″, H-5″); MS m/z 503.2 (MH⁺, 100%); HRMS calcd for C₂₈H₃1N₄O₅ (MH⁺) m/z 503.2302, found 503.2302 (0.7 ppm). HPLC purity 99.6%.

### Example 108: SN39690 Benzyl (4-Chloro-2-methylphenyl)(3-methyl-2-oxo-1-(tetrahydro-2H-pyran-4-yl)-2,3-dihydro-1H-imidazo[4,5-c]pyridin-6-yl)carbamate (134)

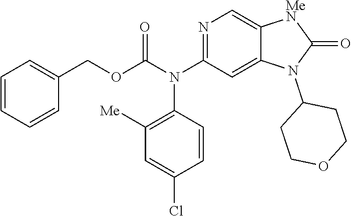 [SMILES: Cc1cc(Cl)ccc1N(C(=O)OCc1ccccc1)c1cc2c(cn1)n(C)c(=O)n2C1CCOCC1]

A solution of benzyl chloroformate (18 μL, 0.13 mmol) in dry THF (2 mL) was added to a stirred solution of imidazopyridinone 122 (40 mg, 0.11 mmol) and iPr₂NEt (26 μL, 150 μmol) in dry THF (5 mL) and the mixture at 20° C. for 16 h. The solvent was evaporated and the residue was partitioned between EtOAc (20 ml) and water (20 mL). The organic fraction was washed with water (20 mL), washed with brine (10 mL), dried (MgSO₄), filtered and the solvent evaporated. The residue was purified by chromatography, eluting with 70% EtOAc/pet. ether, to give carbamate 134 (53 mg, 98%) as a clear glass: mp (EtOAc/pet. ether) 151-154° C.; 1H NMR (CDCl₃) δ 7.90 (s, 1H, H-4′), 7.59 (s, 1H, H-7), 7.30-7.36 (m, 3H, H-3″″, H-4″″, H-5), 7.17-7.27 (m, 4H, H-3″′, H-5″′, H-2″″, H-6), 7.12 (d, J=8.4 Hz, 1H, H-6″′), 5.22 (s, 2H, CH₂O), 4.52 (tt, J=12.4, 4.2 Hz, 1H, H-4″), 4.13 (dd, J=11.6, 4.4 Hz, 2 H, H-2″, H-6″), 3.55 (br t, J=11.9 Hz, 2 H, H-2″, H-6″), 3.39 (s, 3H, 3′-CH₃), 2.42 (dq, J=12.5, 4.5 Hz, 2 H, H-3″, H-5″), 2.14 (s, 3H, 2″′-CH₃), 1.75 (dd, J=12.5, 2.5 Hz, 2 H, H-3″, H-5″); MS m/z 507.2 (MH⁺, 100%), 509.2 (MH⁺, 35%); HRMS calcd for C₂₇H₂₈N₄O₄ (MH⁺) m/z 507.1807, found 507.1803 (0.8 ppm). HPLC purity 99.1%.

### Example 109: SN39872 6-((4-Hydroxy-2-methylphenyl)amino)-3-methyl-1-(tetrahydro-2H-pyran-4-yl)-1,3-dihydro-2H-imidazo[4,5-c]pyridin-2-one (135)

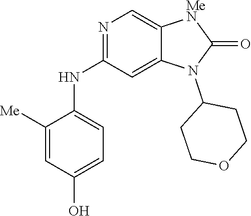 [SMILES: Cc1cc(O)ccc1Nc1cc2c(cn1)n(C)c(=O)n2C1CCOCC1]

A mixture of benzyl ether 124 (172 mg, 0.40 mmol) and Pd/C (20 mg) in a mixture of EtOAc (25 mL) and EtOH (25 mL) was stirred under H2 (50 psi) at 20° C. for 6 h. The mixture was filtered through diatomaceous earth and the filtrate was evaporated. The residue was purified by chromatography, eluting with a gradient (50-100%) of EtOAc/pet. ether, to give imidazopyridinone 135 (44 mg, 32%) as a cream powder: mp (EtOAc/pet ether) 258-261° C.; ¹H NMR (CDCl₃) δ 9.00 (s, 1H, 4″-OH), 7.76 (s, 1H, H-4), 7.56 (s, 1H, 6-NH), 7.22 (d, J=8.5 Hz, 1H, H-6″), 6.16 (d, J=2.7 Hz, 1H, H-3″), 6.54 (dd, J=8.5, 2.8 Hz, 1H, H-5″), 6.51 (s, 1H, H-7), 4.33 (tt, J=12.3, 4.2 Hz, 1H, H-4′), 3.96 (br dd, J=11.4, 4.2 Hz, 2 H, H-2′, H-6′), 3.44 (br t, J=11.3 Hz, 2 H, H-2′, H-6′), 3.25 (s, 3H, 3-CH₃), 2.25 (dq, J=12.4, 4.5 Hz, 2 H, H-3′, H-5′), 2.20 (s, 3H, 2″-CH₃), 1.63 (dd, J=12.4, 2.9 Hz, 2 H, H-3′, H-5′); MS m/z 355.2 (MH⁺, 100%); HRMS calcd for C₁₉H₂₃N₄O₃ (MH⁺) m/z 355.1765, found 355.1782 (−4.9 ppm). HPLC purity 99.4%.

### Example 110: SN40071 7-Methyl-6-((3-methyl-2-oxo-1-(tetrahydro-2H-pyran-4-yl)-2,3-dihydro-1H-imidazo[4,5-c]pyridin-6-yl)amino)imidazo[1,2-a]pyridine-3-carboxylic acid (136)

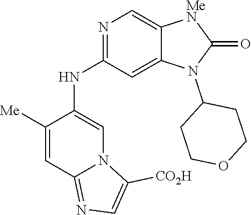 [SMILES: Cc1cc2ncc(C(=O)O)n2cc1Nc1cc2c(cn1)n(C)c(=O)n2C1CCOCC1]

A solution of KOH (130 mg, 2.32 mmol) in water (5 mL) was added to a stirred solution of ethyl 7-methyl-6-((3-methyl-2-oxo-1-(tetrahydro-2H-pyran-4-yl)-2,3-dihydro-1H-imidazo[4,5-c]pyridin-6-yl)amino)imidazo[1,2-a]pyridine-3-carboxylate (129) (209 mg, 0.46 mmol) in MeOH (5 mL) and the mixture was stirred at 40° C. for 4 h. The organic solvent was evaporated and the mixture was cooled at 5° C. and the pH adjusted to 4 with 1 M HCl solution. The resulting precipitate was filtered, washed with cold water (2 mL) and dried to give acid 136 (61 mg, 31%) as a tan powder: ¹H NMR [(CD₃)₂SO] δ 12.66 (br s, 1H, CO₂H), 9.91 (br s, 1H, 6-NH), 8.11 (s, 1H, H-5), 8.01 (s, 1H, H-3), 7.91 (s, 1H, H-4′), 7.62 (s, 1H, H-8), 7.06 (s, 1H, H-7), 4.42 (tt, J=12.3, 4.2 Hz, 1H, H-4″), 4.00 (dd, J=11.4, 4.0 Hz, 2 H, H-2″, H-6″), 3.48 (br t, J=11.2 Hz, 2 H, H-2″, H-6″), 3.31 (s, 3H, 3′-CH₃), 2.40 (dq, J=12.5, 4.5 Hz, 2 H, H-3″, H-5″), 2.40 (s, 3H, 7-CH₃), 1.68 (br dd, J=12.2, 2.6 Hz, 2 H, H-3″, H-5″); MS m/z 423.2 (MH⁺, 100%); HRMS calcd for C₂₁H₂₃N₆₀₄ (MH⁺) m/z 423.1775, found 423.1783 (−1.8 ppm). HPLC purity 98.2%.

### Example 111: SN39667 6-((4-Methoxy-2-methylphenyl)amino)-3-methyl-1-((tetrahydro-2H-pyran-4-yl)methyl)-1,3-dihydro-2H-imidazo[4,5-c]pyridin-2-one (141)

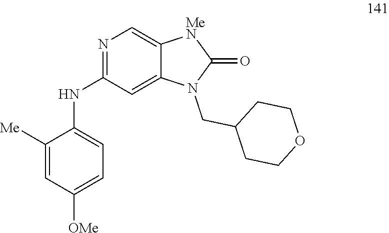 [SMILES: COc1ccc(Nc2cc3c(cn2)n(C)c(=O)n3CC2CCOCC2)c(C)c1]

2-Chloro-5-nitro-N-((tetrahydro-2H-pyran-4-yl)methyl)pyridin-4-amine (137). A solution of (tetrahydro-2H-pyran-4-yl)methanamine (0.73 g, 6.3 mmol) in dry DCM (5 mL) was added dropwise to a stirred solution of nitropyridine 2 (1.02 g, 5.29 mmol) and iPr₂NEt (1.38 mL, 7.94 mmol) in dry DCM (50 mL) at 5° C. The mixture was stirred at 20° C. for 48 h before being diluted with DCM (100 mL) and washed with water (3×50 mL), dried (MgSO₄) and the solvent evaporated. The residue was purified by chromatography, eluting with a gradient (30-50%) of EtOAc/pet. ether, to give chloride 137 (1.39 g, 96%) as yellow crystals: mp 132-134° C.; ¹H NMR (CDCl₃) δ 9.03 (s, 1H, H-6), 8.24 (br s, 1H, 4-NH), 6.74 (s, 1H, H-3), 4.04 (dd, J=11.6, 4.6 Hz, 2 H, H-2″, H-6″), 3.43 (dt, J=11.9, 2.1 Hz, 2 H, H-2″, H-6″), 3.22 (dd, J=6.8, 5.6 Hz, 2 H, H-1′), 1.90-2.02 (m, 1H, H-4″), 1.73 (br d, J=12.9 Hz, 2 H, H-3″, H-5″), 1.43 (ddd, J=13.2, 12.0, 4.6 Hz, 2 H, H-3″, H-5″); MS m/z 272.1 (MH⁺, 100%), 274.1 (MH⁺, 35%); HRMS calcd for C₁₁H₁₅³⁵ClN₃O (MH⁺) m/z 272.0797, found 272.0770 (2.2 ppm).

6-Chloro-N⁴-((tetrahydro-2H-pyran-4-yl)methyl)pyridine-3,4-diamine (138). A solution of nitropyridine 137 (1.30 g, 4.78 mmol) in EtOAc (50 ml) was added dropwise to a stirred suspension of SnCl₂·2H₂O (4.31 g, 19.1 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 138 (1.10 g, 95%) as a white powder: mp 178-180° C.; ¹H NMR (CDCl₃) δ 7.66 (s, 1H, H-2), 6.45 (s, 1H, H-5), 4.43 (br s, 1H, 4-NH), 4.01 (dd, J=10.8, 3.6 Hz, 2 H, H-2″, H-6″), 3.41 (dt, J=11.8, 2.0 Hz, 2 H, H-2″, H-6″), 3.07 (t, J=6.3 Hz, 2 H, H-1′), 2.97 (br s, 2H, 3-NH₂), 1.84-1.92 (m, 1H, H-4″), 1.70 (dd, J=13.0, 1.8 Hz, 2 H, H-3″, H-5″), 1.38-1.45 (ddd, J=13.1, 12.1, 4.5 Hz, 2 H, H-3″, H-5″); MS m/z 228.1 (MH⁺, 100%), 230.1 (MH⁺, 35%); HRMS calcd for C₁₁H₁₇³⁵ClN₃O (MH⁺) m/z 242.1055, found 242.1051 (1.5 ppm).

6-Chloro-1-((tetrahydro-2H-pyran-4-yl)methyl)-1,3-dihydro-2H-imidazo[4,5-c]pyridin-2-one (139). CDI (0.69 g, 4.26 mmol) was added to a stirred solution of diamine 138 (0.86 g, 3.55 mmol) in dry MeCN (50 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was precipitated from 50% EtOAc/pet. ether, to give the pyridinone 139 (0.66 g, 70%) as a white powder: mp 262-264° C.; ¹H NMR [(CD₃)₂SO] δ 11.34 (br s, 1H, 3-H), 7.95 (s, 1H, H-4), 7.46 (s, 1H, H-7), 3.82 (dd, J=11.4, 2.8 Hz, 2 H, H-2″, H-6″), 3.68 (t, J=7.3 Hz, 2 H, H-1′), 3.23 (dt, J=11.6, 1.9 Hz, 2 H, H-2″, H-6″), 1.95-2.05 (m, 1H, H-4″), 1.45 (br dd, J=12.6, 1.9 Hz, 2 H, H-3″, H-5″), 1.27 (ddd, J=12.6, 11.9, 4.4 Hz, 2 H, H-3″, H-5″); MS m/z 268.1 (MH⁺, 100%), 256.1 (MH⁺, 35%); HRMS calcd for C₁₂H₁₅³⁵ClN₃O₂ (MH⁺) m/z 268.0847, found 268.0842 (1.9 ppm).

6-Chloro-3-methyl-1-((tetrahydro-2H-pyran-4-yl)methyl)-1,3-dihydro-2H-imidazo[4,5-c]pyridin-2-one (140). NaH (60% dispersion, 111 mg, 2.78 mmol) was added to a stirred solution of pyridinone 139 (0.62 g, 2.32 mmol) and MeI (0.23 mL, 3.48 mmol) in dry DMF (10 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 140 (245 mg, 37%) as a white powder: mp 185-188° C.; ¹H NMR (CDCl₃) δ 7.98 (s, 1H, H-4), 6.94 (d, J=0.4 Hz, 1H, H-7), 3.98 (dd, J=11.6, 2.7 Hz, 2 H, H-2″, H-6″), 3.73 (d, J=7.3 Hz, 2 H, H-1′), 3.46 (s, 3H, 3-CH₃), 3.34 (dt, J=11.7, 2.2 Hz, 2 H, H-2″, H-6″), 2.04-2.14 (m, 1H, H-4″), 1.56 (br d, J=13.2 Hz, 2 H, H-3″, H-5″), 1.44 (ddd, J=13.2, 11.8, 4.5 Hz, 2 H, H-3″, H-5″); MS m/z 282.1 (MH⁺, 100%), 284.1 (MH⁺, 35%); HRMS calcd for C₁₃H₁₇³⁵ClN₃O₂ (MH⁺) m/z 282.1004, found 282.1003 (0.3 ppm).

6-((4-Methoxy-2-methylphenyl)amino)-3-methyl-1-((tetrahydro-2H-pyran-4-yl)methyl)-1,3-dihydro-2H-imidazo[4,5-c]pyridin-2-one (141). A degassed mixture of chloride 140 (134 mg, 0.48 mmol), 4-methoxy-2-methylaniline (78 mg, 0.57 mmol), Pd₂dba₃ (22 mg, 24 μmol), XPhos (46 mg, 96 μmol) and Cs₂CO₃ (344 mg, 1.06 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 141 (55 mg, 30%) as a tan powder: mp (EtOAc/pet ether) 137-139° C.; ¹H NMR (CDCl₃) δ 7.75 (s, 1H, H-4), 7.21 (d, J=8.6 Hz, 1H, H-6″), 6.84 (d, J=2.9 Hz, 1H, H-3″), 6.78 (dd, J=8.6, 2.9 Hz, 1H, H-5″), 6.00 (d, J=0.4 Hz, 1H, H-7), 5.98 (s, 1H, 6-NH), 3.93 (br dd, J=11.4, 2.9 Hz, 2 H, H-2″, H-6″), 3.83 (s, 3H, 4″-OCH₃), 3.58 (d, J=7.73 Hz, 2 H, H-1′), 3.39 (s, 3H, 3-CH₃), 3.30 (dt, J=11.8, 2.0 Hz, 2 H, H-2″, H-6″), 2.24-2.33 (s, 3H, 2″-CH₃), 1.96-2.06 (m, 1H, H-4″), 1.52 (br d, J=11.9 Hz, 2 H, H-3″, H-5″), 1.35 (dq, J=11.9, 4.4 Hz, 2 H, H-3″, H-5″); MS m/z 383.2 (MH⁺, 100%); HRMS calcd for C₂₁H₂₇N₄O₃ (MH⁺) m/z 383.2078, found 383.2082 (−1.1 ppm). HPLC purity 99.6%.

### Example 112: SN39550 6-((4-Methoxy-2-methylphenyl)amino)-3-methyl-1-(2-(tetrahydro-2H-pyran-4-yl)ethyl)-1,3-dihydro-2H-imidazo[4,5-c]pyridin-2-one (146)

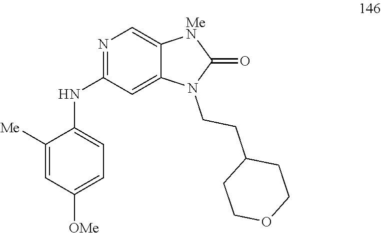 [SMILES: COc1ccc(Nc2cc3c(cn2)n(C)c(=O)n3CCC2CCOCC2)c(C)c1]

2-Chloro-5-nitro-N-(2-(tetrahydro-2H-pyran-4-yl)ethyl)pyridin-4-amine (142). A solution of 2-(tetrahydro-2H-pyran-4-yl)ethan-1-amine (0.61 g, 4.69 mmol) in dry DCM (5 mL) was added dropwise to a stirred solution of nitropyridine 2 (0.82 g, 4.27 mmol) and iPr₂NEt (0.97 mL, 5.55 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (30-50%) of EtOAc/pet. ether, to give chloride 142 (1.16 g, 95%) as yellow crystals: mp 94-95° C.; ¹H NMR (CDCl₃) δ 9.02 (s, 1H, H-6), 8.11 (br s, 1H, 4-NH), 6.72 (s, 1H, H-3), 4.00 (br dd, J=11.6, 4.4 Hz, 2 H, H-2″, H-6″), 3.38 (dt, J=11.8, 1.9 Hz, 2 H, H-2″, H-6″), 1.32-1.36 (m, 2H, H-1′), 1.62-1.74 (m, 5H, H-1″, H-3″, H-5″), 1.32-1.44 (m, 2H, H-3″, H-5″); MS m/z 286.0 (MH⁺, 100%), 288.0 (MH⁺, 35%).

6-Chloro-N⁴-(2-(tetrahydro-2H-pyran-4-yl)ethyl)pyridine-3,4-diamine (143). A solution of nitropyridine 142 (1.13 g, 3.94 mmol) in EtOAc (50 ml) was added dropwise to a stirred suspension of SnCl₂·2H₂O (3.56 g, 15.8 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 143 as a white powder: mp 162-164° C.; ¹H NMR (CDCl₃) δ 7.65 (s, 1H, H-2), 6.44 (s, 1H, H-5), 4.24 (br s, 1H, 4-NH), 3.98 (br dd, J=11.4, 4.3 Hz, 2 H, H-2″, H-6″), 3.40 (dt, J=11.7, 1.8 Hz, 2 H, H-2″, H-6″), 3.18 (dt, J=6.7, 5.8 Hz, 2 H, H-1′), 3.00 (br s, 2H, 3-NH₂), 1.57-1.70 (m, 5H, H-1″, H-3″, H-5″), 1.32-1.42 (m, 2H, H-3″, H-5″); MS m/z 256.1 (MH⁺, 100%), 258.1 (MH⁺, 35%).

6-Chloro-1-(2-(tetrahydro-2H-pyran-4-yl)ethyl)-1,3-dihydro-2H-imidazo[4,5-c]pyridin-2-one (144). CDI (0.47 g, 2.87 mmol) was added to a stirred solution of diamine 143 (0.67 g, 2.60 mmol) in dry MeCN (30 mL) at 20° C. The mixture was stirred at 20° C. for 96 h. The solvent was evaporated and the residue partitioned between CHCl₃ (100 mL) and water (50 mL). The organic fraction was washed with water (2×50 mL), washed with brine (50 mL), dried (MgSO₄), filtered and the solvent evaporated. The residue was precipitated from 50% EtOAc/pet. ether, to give the pyridinone 144 (0.67 g, 92%) as a white powder: mp 212-215° C.; ¹H NMR (CDCl₃) δ 9.42 (br s, 1H, 3-H), 8.12 (s, 1H, H-4), 6.95 (s, 1H, H-7), 3.98 (br dd, J=11.1, 3.6 Hz, 2 H, H-2″, H-6″), 3.90 (t, J=7.6 Hz, 2 H, H-1′), 3.38 (dt, J=11.7, 1.9 Hz, 2 H, H-2″, H-6″), 1.67-1.76 (m, 4H, H-2′, H-3″, H-5″), 1.52-1.62 (m, 1H, H-4″), 1.38 (br dq, J=12.2, 4.4 Hz, 2 H, H-3″, H-5″); MS m/z 282.0 (MH⁺, 100%), 284.0 (MH⁺, 35%).

6-Chloro-3-methyl-1-(2-(tetrahydro-2H-pyran-4-yl)ethyl)-1,3-dihydro-2H-imidazo[4,5-c]pyridin-2-one (145). NaH (60% dispersion, 109 mg, 2.72 mmol) was added to a stirred solution of pyridinone 144 (0.64 g, 2.27 mmol) and MeI (0.22 mL, 3.41 mmol) in dry DMF (10 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 145 (0.51 g, 75%) as a cream powder: mp 138-141° C.; ¹H NMR (CDCl₃) δ 7.99 (s, 1H, H-4), 6.93 (s, 1H, H-7), 3.98 (br dd, J=11.7, 4.4 Hz, 2 H, H-2″, H-6″), 3.89 (t, J=7.5 Hz, 2 H, H-1′), 3.45 (s, 3H, 3-CH₃), 3.37 (dt, J=11.7, 1.9 Hz, 2 H, H-2″, H-6″), 1.65-1.72 (m, 4H, H-2′, H-3″, H-5″), 1.52-1.62 (m, 1H, H-4″), 1.36 (ddd, J=12.5, 11.9, 4.4 Hz, 2 H, H-3″, H-5″); MS m/z 296.0 (MH⁺, 100%), 298.0 (MH⁺, 35%).

6-((4-Methoxy-2-methylphenyl)amino)-3-methyl-1-(2-(tetrahydro-2H-pyran-4-yl)ethyl)-1,3-dihydro-2H-imidazo[4,5-c]pyridin-2-one (146). A degassed mixture of chloride 145 (140 mg, 0.47 mmol), aniline (78 mg, 0.57 mmol), Pd₂dba₃ (22 mg, 24 μmol), XPhos (45 mg, 94 μmol) and Cs₂CO₃ (337 mg, 1.03 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with a gradient (80-100%) of EtOAc/pet. ether, to give imidazopyridinone 146 (53 mg, 28%) as a clear gum: ¹H NMR (CDCl₃) δ 7.75 (s, 1H, H-4), 7.21 (d, J=8.6 Hz, 1H, H-6″′), 6.84 (d, J=2.8 Hz, 1H, H-3″′), 6.77 (dd, J=8.6, 2.9 Hz, 1H, H-5″′), 6.03 (s, 1H, 6-NH), 5.97 (d, J=0.6 Hz, 1H, H-7), 3.92 (br dd, J=11.0, 3.0 Hz, 2 H, H-2″, H-6″), 3.82 (s, 3H, 4″′—OCH₃), 3.74 (t, J=7.2 Hz, 2 H, H-1′), 3.39 (s, 3H, 3-CH₃), 3.31 (dt, J=11.8, 1.9 Hz, 2 H, H-2″, H-6″), 2.24 (s, 3H, 2″′-CH₃), 1.54-1.63 (m, 4H, H-2′, H-3″, H-5″), 1.40-1.48 (m, 1H, H-4″), 1.27 (dq, J=12.1, 4.0 Hz, 2 H, H-3″, H-5″); MS m/z 397.2 (MH⁺, 100%); HRMS calcd for C₂₂H₂₉N₄O₃ (MH⁺) m/z 397.2234, found 397.2241 (−1.8 ppm). HPLC purity 98.9%.

### Example 113: SN39552 6-((4-Chloro-2-methylphenyl)amino)-3-methyl-1-(2-(tetrahydro-2H-pyran-4-yl)ethyl)-1,3-dihydro-2H-imidazo[4,5-c]pyridin-2-one (147)

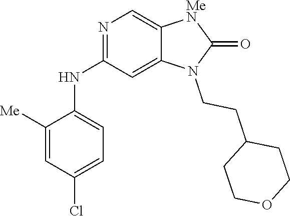 [SMILES: Cc1cc(Cl)ccc1Nc1cc2c(cn1)n(C)c(=O)n2CCC1CCOCC1]

A degassed mixture of chloride 145 (124 mg, 0.42 mmol), 4-chloro-2-methylaniline (71 mg, 0.50 mmol), Pd₂dba₃ (19 mg, 21 μmol), XPhos (40 mg, 84 μmol) and Cs₂CO₃ (301 mg, 0.92 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (80-100% EtOAc/pet. ether) to give imidazopyridinone 147 (110 mg, 65%) as grey crystals: ¹H NMR (CDCl₃) δ 7.81 (d, J=0.6 Hz, 1H, H-4), 7.36 (d, J=8.5 Hz, 1H, H-6″′), 7.24 (d, J=2.4 Hz, 1 H, H-3″′), 7.16 (dd, J=8.5, 2.4 Hz, 1H, H-5″′), 6.26 (d, J=0.6 Hz, 1H, H-7), 6.07 (s, 1H, 6-NH), 3.94 (br dd, J=11.0, 3.3 Hz, 2 H, H-2″, H-6″), 3.80 (dd, J=7.4, 7.0 Hz, 2 H, H-1′), 3.40 (s, 3H, 3-CH₃), 3.34 (dt, J=11.8, 2.0 Hz, 2 H, H-2″, H-6″), 2.26 (s, 3H, 2″′-CH₃), 1.59-1.70 (m, 4H, H-2′, H-3″, H-5″), 1.46-1.56 (m, 1H, H-4″), 1.30 (dq, J=12.3, 4.4 Hz, 2 H, H-3″, H-5″); MS m/z 401.2 (MH⁺, 100%), 403.1 (MH⁺, 35%); HRMS calcd for C₂₁H₂₆³⁵ClN₄O₂ (MH⁺) m/z 401.1739, found 347.1744 (−1.3 ppm). HPLC purity 97.1%.

### Example 114: SN39598 tert-Butyl 4-(6-((4-Methoxy-2-methylphenyl)amino)-3-methyl-2-oxo-2,3-dihydro-1H-imidazo[4,5-c]pyridin-1-yl)piperidine-1-carboxylate (152)

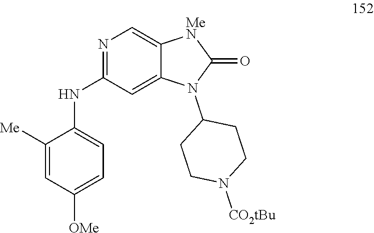 [SMILES: COc1ccc(Nc2cc3c(cn2)n(C)c(=O)n3C2CCN(C(=O)OC(C)(C)C)CC2)c(C)c1]

tert-Butyl 4-((2-Chloro-5-nitropyridin-4-yl)amino)piperidine-1-carboxylate (148). A solution of tert-butyl 4-aminopiperidine-1-carboxylate (1.28 g, 6.37 mmol) in dry DCM (20 mL) was added dropwise to a stirred solution of nitropyridine 2 (1.17 g, 6.06 mmol) and iPr₂NEt (1.27 mL, 7.27 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with 20% EtOAc/pet. ether, to give chloride 148 (1.87 g, 86%) as a yellow powder: mp 146-148° C.; 1H NMR (CDCl₃) δ 9.04 (s, 1H, H-6′), 8.18 (br d, J=7.3 Hz, 1H, 4-NH), 6.75 (s, 1H, H-3′), 4.07 (br d, J=10.2 Hz, 2 H, H-2, H-6), 3.58-3.70 (m, 1H, H-4), 3.04 (br d, J=11.5 Hz, 2 H, H-2, H-6), 2.04 (br dd, J=12.8, 2.9 Hz, 2 H, H-3, H-5), 1.52-1.63 (m, 2H, H-3, H-5), 1.48 (s, 9H, CO₂tBu); MS m/z 257.1 (MH⁺, 100%), 259.1 (MH⁺, 35%).

tert-Butyl 4-((5-Amino-2-chloropyridin-4-yl)amino)piperidine-1-carboxylate (149). A mixture of nitropyridine 148 (2.09 g, 5.86 mmol), Zn powder (3.83 g, 58.6 mmol) and NH₄Cl (3.13 g, 58.6 mmol) in MeOH/THF (1:1, 100 mL) at 20° C. for 2 h. The mixture was filtered through a pad of diatomaceous earth and washed with EtOAc (40 mL). The combined organic fraction was dried (MgSO₄), filtered and the solvent evaporated to give diamine 149 (1.47 g, 69%) as a red powder: mp 171-173° C.; ¹H NMR [(CD₃)₂SO] δ 7.67 (s, 1H, H-6′), 6.45 (s, 1H, H-3′), 4.27 (br d, J=7.2 Hz, 1H, 4′-NH), 4.08 (br s, 2H, 5′-NH₂), 3.39-3.48 (m, 1H, H-4), 2.90-3.04 (m, 4H, H-2, H-6), 1.98-2.08 (m, 2H, H-3, H-5), 1.35-1.48 (m, 11H, H-3, H-5, CO₂tBu); MS m/z 327.1 (MH⁺, 100%), 329.1 (MH⁺, 35%); HRMS calcd for C₁₅H₂₄³⁵ClN₄O₂ (MH⁺) m/z 327.1582, found 327.1584 (−0.4 ppm).

tert-Butyl 4-(6-Chloro-2-oxo-2,3-dihydro-1H-imidazo[4,5-c]pyridin-1-yl)piperidine-1-carboxylate (150). CDI (0.79 g, 4.85 mmol) was added to a stirred solution of diamine 149 (1.44 g, 4.41 mmol) in dry MeCN (50 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was purified by chromatography, eluting with a gradient (60-100%) of EtOAc/pet. ether, to give pyridinone 150 (1.07 g, 69%) as a white powder: mp 210-212° C.; ¹H NMR (CDCl₃) δ 11.35 (br s, 1H, 3′-H), 7.95 (d, J=0.4 Hz, 1H, H-4′), 7.44 (d, J=0.4 Hz, 1H, H-7′), 4.33 (tt, J=12.2, 4.0 Hz, 1 H, H-4), 4.07 (br d, J=11.0 Hz, 2 H, H-2, H-6), 2.73-2.90 (m, 2H, H-2, H-6), 2.16 (dq, J=12.5, 4.4 Hz, 2 H, H-3, H-5), 1.70 (br d, J=11.8 Hz, 2 H, H-3, H-5), 1.48 (s, 9H, CO₂tBu); MS m/z 353.1 (MH⁺, 100%), 355.1 (MH⁺, 35%); HRMS calcd for C₁₆H₂₂³⁵ClN₄O₃ (MH⁺) m/z 353.1375, found 353.1374 (−0.2 ppm).

tert-Butyl 4-(6-Chloro-3-methyl-2-oxo-2,3-dihydro-1H-imidazo[4,5-c]pyridin-1-yl)piperidine-1-carboxylate (151). NaH (60% dispersion, 214 mg, 5.34 mmol) was added to a stirred solution of pyridinone 150 (1.57 g, 4.45 mmol) and MeI (0.42 mL, 6.68 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 151 (1.51 g, 92%) as a white powder: mp 218-220° C.; ¹H NMR (CDCl₃) δ 7.99 (s, 1H, H-4′), 7.05 (d, J=0.4 Hz, 1H, H-7), 4.42 (tt, J=12.6, 4.2 Hz, 1H, H-4), 4.32 (br s, 2H, H-2, H-6), 3.45 (s, 3H, 3-CH₃), 2.85 (br dd, J=12.5, 11.8 Hz, 2 H, H-2, H-6), 2.20 (dq, J=12.7, 4.6 Hz, 2 H, H-3, H-5), 1.81 (br d, J=12.1 Hz, 2 H, H-3, H-5), 1.51 (s, 9H, CO₂tBu); MS m/z 367.0 (MH⁺, 100%), 369.0 (MH⁺, 35%); HRMS calcd for C₁₇H₂₄³⁵ClN₄O₃ (MH⁺) m/z 367.1531, found 367.1535 (−0.9 ppm).

tert-Butyl 4-(6-((4-Methoxy-2-methylphenyl)amino)-3-methyl-2-oxo-2,3-dihydro-1H-imidazo[4,5-c]pyridin-1-yl)piperidine-1-carboxylate (152). A degassed mixture of chloride 151 (185 mg, 0.50 mmol), 4-methoxy-2-methylaniline (83 mg, 0.61 mmol), Pd₂dba₃ (23 mg, 25 μmol), XPhos (48 mg, 100 μmol) and Cs₂CO₃ (358 mg, 1.10 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 152 (177 mg, 76%) as a brown gum: ¹H NMR (CDCl₃) δ 7.77 (s, 1H, H-4′), 7.23 (d, J=8.6 Hz, 1H, H-6″), 6.82 (d, J=2.9 Hz, 1H, H-3″), 6.76 (dd, J=8.6, 2.9 Hz, 1H, H-5″), 6.17 (s, 1H, H-7′), 5.95 (br s, 1H, 6′-NH), 4.20-4.30 (m, 3H, H-2, H-4, H-6), 3.80 (s, 3H, 4″-OCH₃), 3.38 (s, 3H, 3′-CH₃), 2.75-2.82 (m, 2H, H-2, H-6), 2.25 (s, 3H, 2″-CH₃), 2.10 (dq, J=12.6, 4.5 Hz, 2 H, H-3, H-5), 1.74 (br d, J=12.6 Hz, 2 H, H-3, H-5), 1.45 (s, 9H, CO₂tBu); MS m/z 468.2 (MH⁺, 100%); HRMS calcd for C₂₅H34N₅04 (MH⁺) m/z 468.2605, found 468.2612 (−1.5 ppm). HPLC purity 97.8%.

### Example 115: SN39600 6-((4-Methoxy-2-methylphenyl)amino)-3-methyl-1-(piperidin-4-yl)-1,3-dihydro-2H-imidazo[4,5-c]pyridin-2-one hydrochloride (153)

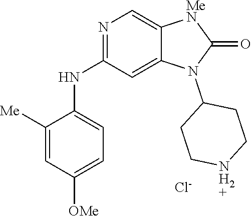 [SMILES: COc1ccc(Nc2cc3c(cn2)n(C)c(=O)n3C2CC[NH2+]CC2)c(C)c1 ; [Cl-]]

A mixture of carbamate 152 (114 mg, 0.24 mmol) and 4 M HCl in dioxane (0.61 mL, 2.44 mmol) in MeOH (2 mL) was stirred at 20° C. for 16 h. The mixture was cooled, diluted with water (10 mL) and the pH adjusted to 7 with aqueous NaHCO₃ solution and chilled for 1 h. The precipitate was filtered and washed with water (2 mL) and dried to give imidazopyridinone 153 (109 mg, 81%) as a tan powder: ¹H NMR (CDCl₃) δ 7.76 (s, 1H, H-4), 7.28 (d, J=8.6 Hz, 1 H, H-6″), 6.83 (d, J=2.9 Hz, 1H, H-3″), 6.78 (dd, J=8.6, 2.9 Hz, 1H, H-5″), 6.31 (s, 1H, H-7), 5.93 (br s, 1H, 6-NH), 4.24 (tt, J=12.4, 4.1 Hz, 1H, H-4′, 3.82 (s, 3H, 4″-OCH₃), 3.38 (s, 3H, 3-CH₃), 3.20 (br d, J=12.4 Hz, 2 H, H-2′, H-6′), 2.73 (dt, J=12.5, 2.3 Hz, 2 H, H-2′, H-6′), 2.26 (s, 3H, 2″-CH₃), 2.13 (dq, J=12.5, 4.2 Hz, 2 H, H-3′, H-5′), 1.76 (br d, J=11.9 Hz, 2 H, H-3′, H-5′), 1′-NH not observed; MS m/z 468.2 (MH⁺, 100%); HRMS calcd for C₂₀H₂₆N₅O₂ (MH⁺) m/z 368.2081, found 368.2077 (1.1 ppm). HPLC purity 97.0%.

### Example 116: SN39686 6-((4-Methoxy-2-methylphenyl)amino)-3-methyl-1-(1-methylpiperidin-4-yl)-1,3-dihydro-2H-imidazo[4,5-c]pyridin-2-one hydrochloride (157)

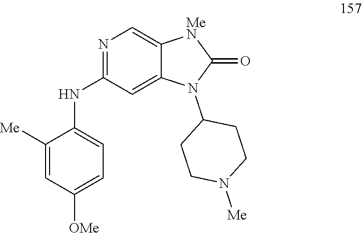 [SMILES: COc1ccc(Nc2cc3c(cn2)n(C)c(=O)n3C2CCN(C)CC2)c(C)c1]

tert-Butyl 4-(6-(((Benzyloxy)carbonyl)(4-methoxy-2-methylphenyl)amino)-3-methyl-2-oxo-2,3-dihydro-1H-imidazo[4,5-c]pyridin-1-yl)piperidine-1-carboxylate (154). A solution of benzyl chloroformate (142 μL, 1.00 mmol) in dry THF (2 mL) was added to a stirred solution of imidazopyridinone 152 (424 mg, 0.91 mmol) and iPr₂NEt (190 μL, 1.09 mmol) in dry THF (20 mL) and the mixture at 20° C. for 16 h. The solvent was evaporated and the residue was partitioned between EtOAc (100 ml) and water (100 mL). The organic fraction was washed with water (20 mL), washed with brine (10 mL), dried (MgSO₄), filtered and the solvent evaporated. The residue was purified by chromatography, eluting with a gradient (80-100%) of EtOAc/pet. ether, to give carbamate 154 (510 mg, 93%) as a clear gum: ¹H NMR (CDCl₃) δ 7.92 (s, 1H, H-4′), 7.52 (s, 1H, H-7), 7.26-7.34 (m, 3H, H-2″′, H-4″′, H-6″′), 7.18-7.23 (m 2 H, H-3″′, H-5″′), 7.11 (d, J=8.5 Hz, 1H, H-6″), 6.79 (d, J=2.9 Hz, 1H, H-3″), 6.75 (dd, J=8.5, 2.9 Hz, 1H, H-5″), 5.20 (s, 2H, CH₂O), 4.27-4.40 (m, 3H, H-2, H-4, H-6), 3.81 (s, 3H, 4″-OCH₃), 3.38 (s, 3H, 3′-CH₃), 2.84 (br t, J=11.8 Hz, 2 H, H-2, H-6), 2.27 (dq, J=12.5, 4.5 Hz, 2 H, H-3, H-5), 2.15 (s, 3H, 2″-CH₃), 1.79 (br d, J=12.4 Hz, 2 H, H-3, H-5), 1.52 (s, 9H, CO₂tBu); MS m/z 602.2 (MH⁺, 100%).

Benzyl (4-Methoxy-2-methylphenyl)(3-methyl-2-oxo-1-(piperidin-4-yl)-2,3-dihydro-1H-imidazo[4,5-c]pyridin-6-yl)carbamate (155). A mixture of carbamate 154 (510 mg, 0.85 mmol) and 1.25 M HCl in MeOH (6.9 mL, 8.5 mmol) in MeOH (10 mL) was stirred at 20° C. for 16 h. The mixture was cooled, diluted with water (10 mL) and the pH adjusted to 7 with aqueous NaHCO₃ solution and extracted with CHCl₃ (3×20 mL). The combined organic fraction was dried (MgSO₄) and the solvent evaporated to give carbamate 155 (109 mg, 81%) as a tan powder: ¹H NMR (CDCl₃) δ 7.92 (s, 1H, H-4′), 7.59 (s, 1H, H-7), 7.20-7.32 (m, 3H, H-2″′, H-4″′, H-6″′), 7.20-7.24 (m 2 H, H-3″′, H-5″′), 7.13 (d, J=8.5 Hz, 1H, H-6″), 6.78 (d, J=2.9 Hz, 1 H, H-3″), 6.75 (dd, J=8.5, 2.9 Hz, 1H, H-5″), 5.22 (s, 2H, CH₂O), 4.36 (tt, J=12.4, 4.1 Hz, 1H, H-4′), 3.80 (s, 3H, 4″-OCH₃), 3.38 (s, 3H, 3-CH₃), 3.26 (br d, J=12.2 Hz, 2 H, H-2, H-6), 2.77 (dt, J=12.4, 2.1 Hz, 2 H, H-2, H-6), 2.24 (dq, J=12.5, 4.1 Hz, 2 H, H-3, H-5), 2.17 (s, 3H, 2″-CH₃), 1.81 (br dd, J=11.9, 2.1 Hz, 2 H, H-3, H-5), 1-NH not observed; MS m/z 502.2 (MH⁺, 100%).

Benzyl (4-Methoxy-2-methylphenyl)(3-methyl-1-(1-methylpiperidin-4-yl)-2-oxo-2,3-dihydro-1H-imidazo[4,5-c]pyridin-6-yl)carbamate (156). NaBH(OAc)₃ (550 mg, 2.58 mmol) was added to a stirred mixture of amine 155 (430 mg, 0.86 mmol) and formaldehyde (37%, 0.13 mL, 1.71 mmol) in DCM (10 mL) and the mixture was stirred at 20° C. for 16 h. The mixture was partitioned between aqueous NaHCO₃ solution (30 mL) and DCM (80 mL). The organic fraction was washed with aqueous NaHCO₃(2×20 mL), brine (30 mL) and dried (MgSO₄) and the solvent evaporated. The residue was purified by chromatography, eluting with EtOAc, to give imidazopyridinone 156 (177 mg, 76%) as a tan powder: ¹H NMR (CDCl₃) δ 7.92 (s, 1H, H-4′), 7.59 (s, 1H, H-7), 7.27-7.32 (m, 5H, H-2″″, H-3″″, H-4″″, H-5″″, H-6), 7.14 (d, J=8.5 Hz, 1H, H-6″′), 6.78 (d, J=2.9 Hz, 1H, H-3″′), 6.74 (dd, J=8.5, 2.9 Hz, 1H, H-5″′), 5.21 (s, 2H, CH₂O), 4.31 (tt, J=12.5, 4.2 Hz, 1H, H-4″), 3.80 (s, 3H, 4″′—OCH₃), 3.38 (s, 3H, 3′-CH₃), 3.02 (br d, J=11.7 Hz, 2 H, H-2″, H-6″), 2.33-2.45 (m, 5H, H-2″, H-6″, 4″-CH₃), 2.10-2.19 (m, 5H, H-3″, H-5″, 2″′-CH₃), 1.79 (br d, J=11.9 Hz, 2 H, H-3″, H-5″); MS m/z 516.2 (MH⁺, 100%). HRMS calcd for C₂₉H34N₅04 (MH⁺) m/z 516.2605, found 516.2610 (−1.0 ppm).

6-((4-Methoxy-2-methylphenyl)amino)-3-methyl-1-(1-methylpiperidin-4-yl)-1,3-dihydro-2H-imidazo[4,5-c]pyridin-2-one (157). A mixture of benzyl ether 156 (350 mg, 0.68 mmol) and Pd/C (50 mg) in EtOH (50 mL) was stirred under Hz (50 psi) at 20° C. for 4 h. The mixture was filtered through diatomaceous earth and the filtrate was evaporated. The residue was purified by chromatography, eluting with a gradient (50-100%) of EtOAc/pet. ether, to give imidazopyridinone 157 (250 mg, 97%) as a white powder: mp (EtOAc/pet ether) 128-131° C.; ¹H NMR (CDCl₃) δ 7.76 (d, J=0.4 Hz, 1H, H-4), 7.29 (d, J=8.4 Hz, 1H, H-6″), 6.82 (d, J=2.9 Hz, 1H, H-3″), 6.77 (dd, J=8.4, 2.9 Hz, 1H, H-5″), 6.35 (s, 1H, H-7), 5.81 (br s, 1H, 6-NH), 4.20 (tt, J=12.5, 4.2 Hz, 1H, H-4′), 3.82 (s, 3H, 4″-OCH₃), 3.38 (s, 3H, 3-CH₃), 2.95 (br d, J=11.7 Hz, 2 H, H-2′, H-6′), 2.22-2.34 (m, 8H, 1′-CH₃, H-2′, H-6′, 2″-CH₃), 2.10 (dq, J=12.0, 2.1 Hz, 2 H, H-3′, H-5′), 1.74 (br d, J=11.9 Hz, 2 H, H-3′, H-5′); MS m/z 382.2 (MH⁺, 100%). HRMS calcd for C₂₁H₂₈N₅O₂ (MH⁺) m/z 382.2238, found 382.2236 (0.5 ppm).

### Example 117: SN39627 tert-Butyl 4-((6-((4-Methoxy-2-methylphenyl)amino)-3-methyl-2-oxo-2,3-dihydro-1H-imidazo[4,5-c]pyridin-1-yl)methyl)piperidine-1-carboxylate (162)

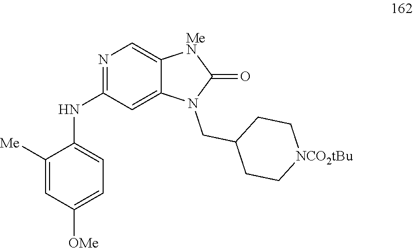

tert-Butyl 4-(((2-Chloro-5-nitropyridin-4-yl)amino)methyl)piperidine-1-carboxylate (158). A solution of tert-butyl 4-(aminomethyl)piperidine-1-carboxylate (1.35 g, 6.28 mmol) in dry DCM (20 mL) was added dropwise to a stirred solution of nitropyridine 2 (1.10 g, 5.71 mmol) and iPr₂NEt (1.29 mL, 7.42 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (20-40%) of EtOAc/pet. ether, to give chloride 158 (2.04 g, 96%) as a yellow powder: mp 145-147° C.; ¹H NMR (CDCl₃) δ 9.03 (s, 1H, H-6″), 8.25 (br s, 1H, 4″-NH), 6.73 (s, 1H, H-3″), 4.18 (br s, 2H, H-2, H-6), 3.22 (dd, J=6.3, 6.0 Hz, 2 H, H-1′), 2.47 (br t, J=12.4 Hz, 2 H, H-2, H-6), 1.83-1.90 (m, 1H, H-4), 1.78 (br d, J=13.9 Hz, 2 H, H-3, H-5), 1.47 (s, 9H, CO₂tBu), 1.20-1.30 (m, 2H, H-3, H-5); MS m/z 371.1 (MH⁺, 100%), 373.1 (MH⁺, 35%); HRMS calcd for C₁₆H₂₄³⁵ClN₄O₄ (MH⁺) m/z 371.1494, found 371.1494 (0.6 ppm).

tert-Butyl 4-(((5-Amino-2-chloropyridin-4-yl)amino)methyl)piperidine-1-carboxylate (159). A mixture of nitropyridine 158 (1.19 g, 3.21 mmol), Zn powder (2.10 g, 32.1 mmol) and NH₄Cl (1.72 g, 32.1 mmol) in MeOH/THF (1:1, 100 mL) at 20° C. for 4 h. The mixture was filtered through a pad of diatomaceous earth and washed with EtOAc (50 mL). The combined organic fraction was dried (MgSO₄), filtered and the solvent evaporated to give diamine 159 (1.09 g, 100%) as a brown oil: ¹H NMR (CDCl₃) δ 7.76 (s, 1H, H-6″), 6.41 (s, 1H, H-3″), 4.60 (br s, 1H, 4″-NH), 4.08-4.20 (m, 2H, H-2, H-6), 3.07 (br t, J=6.1 Hz, 2 H, H-1′), 2.72 (br dd, J=12.0, 11.5 Hz, 2 H, H-2, H-6), 1.73-1.86 (m, 5H, H-3, H-4, H-5, 5″-NH₂), 1.47 (s, 9H, CO₂tBu), 1.15-1.26 (m, 2H, H-3, H-5); MS m/z 341.1 (MH⁺, 100%), 343.1 (MH⁺, 35%); HRMS calcd for C₁₆H₂₆³⁵ClN₄O₂ (MH⁺) m/z 341.1739, found 341.1747 (−2.4 ppm).

tert-Butyl 4-((6-Chloro-2-oxo-2,3-dihydro-1H-imidazo[4,5-c]pyridin-1-yl)methyl)piperidine-1-carboxylate (160). CDI (1.04 g, 6.41 mmol) was added to a stirred solution of diamine 159 (1.09 g, 4.41 mmol) in dry MeCN (60 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was purified by chromatography, eluting with a gradient (0-5%) of MeOH/DCM, to give pyridinone 160 (1.71 g, 87%) as a white powder: mp 240-242° C.; ¹H NMR (CDCl₃) δ 11.33 (br s, 1 H, 3″-H), 7.95 (s, 1H, H-4″), 7.46 (s, 1H, H-7), 3.92 (br d, J=11.6 Hz, 2 H, H-2, H-6), 3.68 (d, J=7.3 Hz, 2 H, H-1′), 2.60-2.70 (m, 2H, H-2, H-6), 1.90-2.00 (m, 1H, H-4), 1.51 (br d, J=11.3 Hz, 2 H, H-3, H-5), 1.38 (s, 9H, CO₂tBu), 1.14 (dq, J=12.4, 4.3 Hz, 2 H, H-3, H-5); MS m/z 367.1 (MH⁺, 100%), 369.1 (MH⁺, 35%); HRMS calcd for C₁₇H₂₄³⁵ClN₄O₃ (MH⁺) m/z 367.1531, found 367.1537 (−1.6 ppm).

tert-Butyl 4-((6-Chloro-3-methyl-2-oxo-2,3-dihydro-1H-imidazo[4,5-c]pyridin-1-yl)methyl)piperidine-1-carboxylate (161). NaH (60% dispersion, 80 mg, 2.00 mmol) was added to a stirred solution of pyridinone 160 (0.61 g, 1.66 mmol) and MeI (0.16 mL, 2.49 mmol) in dry DMF (5 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 161 (0.56 g, 89%) as a white powder: mp 194-196° C.; ¹H NMR (CDCl₃) δ 7.98 (s, 1H, H-4″), 6.93 (s, 1H, H-7), 4.08-4.18 (m, 2H, H-2, H-6), 3.72 (d, J=7.3 Hz, 2 H, H-1′), 2.66 (br t, J=12.4 Hz, 2 H, H-2, H-6), 3.46 (s, 3H, 3″-CH₃), 2.66 (br t, J=12.4 Hz, 2 H, H-2, H-6), 1.96-2.04 (m, 1H, H-4), 1.62 (br d, J=12.2 Hz, 2 H, H-3, H-5), 1.45 (s, 9H, CO₂tBu), 1.25 (dq, J=12.4, 4.3 Hz, 2 H, H-3, H-5); MS m/z 381.0 (MH⁺, 100%), 381.0 (MH⁺, 35%); HRMS calcd for C₁₈H₂₆³⁵ClN₄O₃ (MH⁺) m/z 381.1688, found 381.1698 (−2.7 ppm).

tert-Butyl 4-((6-((4-Methoxy-2-methylphenyl)amino)-3-methyl-2-oxo-2,3-dihydro-1H-imidazo[4,5-c]pyridin-1-yl)methyl)piperidine-1-carboxylate (162). A degassed mixture of chloride 161 (674 mg, 1.77 mmol), 4-methoxy-2-methylaniline (291 mg, 2.12 mmol), Pd₂dba₃ (81 mg, 89 μmol), XPhos (169 mg, 350 μmol) and Cs₂CO₃ (1.27 g, 3.89 mmol) in MeCN (20 mL) was stirred in a sealed tube at 120° C. for 16 h. The mixture was cooled, diluted with EtOAc (80 mL), filtered through diatomaceous earth and the filtrate was evaporated. The residue was partitioned between EtOAc (80 ml) and water (80 mL). The organic fraction was washed with water (50 mL), washed with brine (50 mL), dried (MgSO₄), filtered and the solvent evaporated. The residue was purified by chromatography, eluting with EtOAc, to give imidazopyridinone 162 (470 mg, 55%) as a tan foam: mp 86-89° C.; ¹H NMR (CDCl₃) δ 7.76 (d, J=0.5 Hz, 1H, H-4″), 7.22 (d, J=8.6 Hz, 1H, H-6), 6.84 (d, J=2.9 Hz, 1 H, H-3″′), 6.78 (dd, J=8.6, 2.9 Hz, 1H, H-5″′), 5.99 (d, J=0.5 Hz, 1H, H-7), 5.98 (br s, 1H, 6″-NH), 4.03-4.10 (m, 2H, H-2, H-6), 3.82 (s, 3H, 4″′—OCH₃), 3.58 (d, J=7.0 Hz, 2 H, H-1′), 3.39 (s, 3H, 3″-CH₃), 2.62 (br t, J=12.1 Hz, 2 H, H-2, H-6), 2.24 (s, 3H, 2″′-CH₃), 1.87-1.94 (m, 1H, H-4), 1.56 (br d, J=13.4 Hz, 2 H, H-3, H-5), 1.45 (s, 9H, CO₂tBu), 1.15 (dq, J=12.4, 4.2 Hz, 2 H, H-3, H-5); MS m/z 482.2 (MH⁺, 100%); HRMS calcd for C₂₆H36N₅O₄ (MH⁺) m/z 482.2762, found 482.2765 (−0.6 ppm). HPLC purity 99.0%.

### Example 118: SN39628 6-((4-Methoxy-2-methylphenyl)amino)-3-methyl-1-(piperidin-4-ylmethyl)-1,3-dihydro-2H-imidazo[4,5-c]pyridin-2-one dihydrochloride (163)

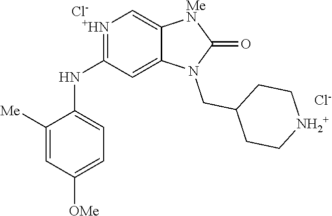 [SMILES: COc1ccc(Nc2cc3c(c[nH+]2)n(C)c(=O)n3CC2CC[NH2+]CC2)c(C)c1 ; [Cl-] ; [Cl-]]

A mixture of carbamate 162 (124 mg, 0.23 mmol) and 4 M HCl in dioxane (0.60 mL, 2.32 mmol) in MeOH (2 mL) was stirred at 20° C. for 16 h. The solvent was evaporated to give imidazopyridinone dihydrochloride 163 (105 mg, 100%) as a tan powder: ¹H NMR [(CD₃)₂SO] δ 13.10 (br s, 1H, NH₂⁺Cl⁻), 9.85 (br s, 1H, NH₂⁺Cl⁻), 9.16 (br s, 1H, NH₂⁺Cl⁻), 8.91 (br s, 1H, NH₂⁺Cl⁻), 7.86 (s, 1H, H-4), 7.20 (d, J=8.6 Hz, 1H, H-6″′), 6.98 (d, J=2.8 Hz, 1H, H-3″′), 6.89 (dd, J=8.6, 2.9 Hz, 1H, H-5″′), 6.68 (s, 1H, H-7), 3.80 (s, 3H, 4″′—OCH₃), 3.74 (br d, J=6.3 Hz, 2 H, H-1″), 3.38 (s, 3H, 3-CH₃), 3.22 (br d, J=12.3 Hz, 2 H, H-2′, H-6′), 2.73-2.83 (m, 2H, H-2′, H-6′), 2.18 (s, 3H, 2″′-CH₃), 1.77-1.84 (m, 1H, H-4), 1.73 (br d, J=11.9 Hz, 2 H, H-3′, H-5′), 1.38-1.49 (m, 2H, H-3′, H-5′); MS m/z 382.2 (MH⁺, 100%); HRMS calcd for C₂₁H₂₈N₅O₂ (MH⁺) m/z 382.2238, found 382.2235 (0.7 ppm). HPLC purity 99.2%.

### Example 119: SN39687 6-((4-Methoxy-2-methylphenyl)amino)-3-methyl-1-((1-methylpiperidin-4-yl)methyl)-1,3-dihydro-2H-imidazo[4,5-c]pyridin-2-one (167)

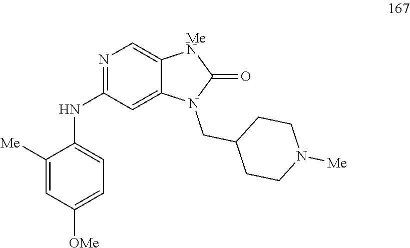 [SMILES: COc1ccc(Nc2cc3c(cn2)n(C)c(=O)n3CC2CCN(C)CC2)c(C)c1]

tert-Butyl 4-((6-(((benzyloxy)carbonyl)(4-methoxy-2-methylphenyl)amino)-3-methyl-2-oxo-2,3-dihydro-1H-imidazo[4,5-c]pyridin-1-yl)methyl)piperidine-1-carboxylate (164). A solution of benzyl chloroformate (109 μL, 0.77 mmol) in dry THF (2 mL) was added to a stirred solution of imidazopyridinone 162 (336 mg, 0.70 mmol) and iPr₂NEt (146 μL, 0.84 mmol) in dry THF (25 mL) and the mixture at 20° C. for 16 h. The solvent was evaporated and the residue was partitioned between EtOAc (100 ml) and water (100 mL). The organic fraction was washed with water (20 mL), washed with brine (10 mL), dried (MgSO₄), filtered and the solvent evaporated. The residue was purified by chromatography, eluting with a gradient (70-100%) of EtOAc/pet. ether, to give carbamate 164 (378 mg, 88%) as a tan gum: ¹H NMR (CDCl₃) δ 7.91 (s, 1H, H-4″), 7.52 (s, 1H, H-7), 7.28-7.33 (m, 3H, H-2″″, H-4″″, H-6), 7.20-7.24 (m 2 H, H-3″″, H-5), 7.11 (d, J=8.5 Hz, 1H, H-6″′), 6.80 (d, J=2.9 Hz, 1H, H-3″′), 6.75 (dd, J=8.5, 2.9 Hz, 1H, H-5″′), 5.22 (s, 2H, CH₂O), 4.05-4.16 (m, 2H, H-2, H-6), 3.81 (s, 3H, 4″′—OCH₃), 3.71 (br d, J=7.0 Hz, 2 H, H-1′), 3.398 (s, 3 H, 3″-CH₃), 2.67 (br t, J=12.0 Hz, 2 H, H-2, H-6), 2.22 (s, 3H, 2″′-CH₃), 1.95-2.04 (m, 1H, H-4), 1.62 (br d, J=12.1 Hz, 2 H, H-3, H-5), 1.45 (s, 9H, CO₂tBu), 1.25 (dq, J=12.4, 4.0 Hz, 2 H, H-3, H-5); MS m/z 616.1 (MH⁺, 100%).

Benzyl (4-Methoxy-2-methylphenyl)(3-methyl-2-oxo-1-(piperidin-4-ylmethyl)-2,3-dihydro-1H-imidazo[4,5-c]pyridin-6-yl)carbamate (165). A mixture of carbamate 164 (450 mg, 0.61 mmol) and 1.25 M HCl in MeOH (10 mL, 12.5 mmol) was stirred at 20° C. for 16 h. The mixture was cooled, diluted with water (10 mL) and the pH adjusted to 7 with aqueous NaHCO₃ solution and extracted with CHCl₃ (3×20 mL). The combined organic fraction was dried (MgSO₄) and the solvent evaporated to give carbamate 165 (316 mg, 100%) as a tan powder: ¹H NMR (CDCl₃) δ 7.91 (s, 1H, H-4), 7.35 (s, 1H, H-7), 7.28-7.34 (m, 3H, H-2″″, H-4″″, H-6), 7.30-7.33 (m, 2H, H-3″″, H-5), 7.12 (d, J=8.5 Hz, 1H, H-6″′), 6.78 (d, J=2.9 Hz, 1H, H-3″′), 6.75 (dd, J=8.5, 2.9 Hz, 1H, H-5″′), 5.20 (s, 2H, CH₂O), 3.81 (s, 3H, 4″′-OCH₃), 3.71 (d, J=7.2 Hz, 2 H, H-1′), 3.38 (s, 3H, 3-CH₃), 3.10 (br d, J=12.3 Hz, 2 H, H-2″, H-6″), 2.57 (dt, J=12.2, 2.3 Hz, 2 H, H-2″, H-6″), 2.13 (s, 3H, 2″′-CH₃), 1.93-2.03 (m, 1H, H-4″), 1.65 (br d, J=13.0 Hz, 2 H, H-3″, H-5″) 1.27 (dq, J=12.2, 3.9 Hz, 2 H, H-3″, H-5″), 1″-NH not observed; MS m/z 516.2 (MH⁺, 100%); HRMS calcd for C₂₉H3₆N₅₀₄ (MH⁺) m/z 516.2605, found 516.2612 (−1.3 ppm).

Benzyl (4-Methoxy-2-methylphenyl)(3-methyl-1-((1-methylpiperidin-4-yl)methyl)-2-oxo-2,3-dihydro-1H-imidazo[4,5-c]pyridin-6-yl)carbamate (166). NaBH(OAc)₃ (381 mg, 1.80 mmol) was added to a stirred mixture of amine 165 (310 mg, 0.60 mmol) and formaldehyde (37%, 0.09 mL, 1.20 mmol) in DCM (10 mL) and the mixture was stirred at 20° C. for 16 h. The mixture was partitioned between aqueous NaHCO₃ solution (30 mL) and DCM (80 mL). The organic fraction was washed with aqueous NaHCO₃(2×20 mL), brine (30 mL) and dried (MgSO₄) and the solvent evaporated. The residue was purified by chromatography, eluting with a gradient (0-10%) of MeOH/DCM, to give imidazopyridinone 166 (286 mg, 90%) as a clear oil: ¹H NMR (CDCl₃) δ 7.92 (d, J=0.3 Hz, 1H, H-4), 7.33 (s, 1H, H-7), 7.28-7.32 (m, 3H, H-2″″, H-2″″, H-6), 7.28-7.32 (m, 2H, H-3″″, H-5), 7.12 (d, J=8.5 Hz, 1H, H-6″′), 6.79 (d, J=2.9 Hz, 1H, H-3″′), 6.75 (dd, J=8.5, 2.9 Hz, 1H, H-5″′), 5.21 (s, 2H, CH₂O), 3.81 (s, 3H, 4″′—OCH₃), 3.74 (d, J=7.1 Hz, 2 H, H-1′), 3.39 (s, 3H, 3-CH₃), 2.97 (br d, J=9.4 Hz, 2 H, H-2″, H-6″), 2.37 (s, 3H, 1″-CH₃), 2.04-2.13 (m, 5H, H-2″, H-6″, 2″′-CH₃), 3.74 (d, J=7.1 Hz, 2 H, H-1′), 1.88-1.94 (m, 1H, H-4″), 1.72 (br d, J=12.3 Hz, 2 H, H-3″, H-5″), 1.28 (br d, J=10.6 Hz, 2 H, H-3″, H-5″); MS m/z 530.2 (MH⁺, 100%); HRMS calcd for C₃₀H3₆N₅₀₄ (MH⁺) m/z 530.2762, found 530.2768 (−1.1 ppm). 6-((4-Methoxy-2-methylphenyl)amino)-3-methyl-1-((1-methylpiperidin-4-yl)methyl)-1,3-dihydro-2H-imidazo[4,5-c]pyridin-2-one (167). A mixture of carbamate 166 (280 mg, 0.53 mmol) and Pd/C (50 mg) in EtOH (80 mL) was stirred under Hz (50 psi) at 20° C. for 4 h. The mixture was filtered through diatomaceous earth and the filtrate was evaporated to give imidazopyridinone 167 (210 mg, 100%) as a clear glass: ¹H NMR (CDCl₃) δ 7.74 (d, J=0.5 Hz, 1H, H-4), 7.21 (d, J=8.6 Hz, 1H, H-6″′), 6.84 (d, J=2.9 Hz, 1H, H-3″′), 6.78 (dd, J=8.6, 2.9 Hz, 1H, H-5″′), 6.03 (br s, 1H, 6-NH), 6.00 (s, 1H, H-7), 3.83 (s, 3H, 4″′—OCH₃), 3.58 (d, J=7.21 Hz, 2 H, H-1′), 3.38 (s, 3H, 3-CH₃), 2.94 (br d, J=11.8 Hz, 2 H, H-2″, H-6″), 2.34 (s, 3H, 1″-CH₃), 2.24 (s, 3H, 2″′-CH₃), 2.03 (br t, J=11.2 Hz, 2 H, H-2″, H-6″), 1.78-1.88 (m, 1H, H-4″), 1.66 (br d, J=12.1 Hz, 2 H, H-3″, H-5″), 1.42 (dq, J=12.0, 3.7 Hz, 2 H, H-3″, H-5″); MS m/z 396.2 (MH⁺, 100%). HRMS calcd for C₂₂H₃₀N₅O₂ (MH⁺) m/z 396.2394, found 396.2396 (−0.6 ppm).

### Example 120: SN39540 6-((4-Methoxy-2-methylphenyl)amino)-1-(4-methoxycyclohexyl)-3-methyl-1,3-dihydro-2H-imidazo[4,5-c]pyridin-2-one (172)

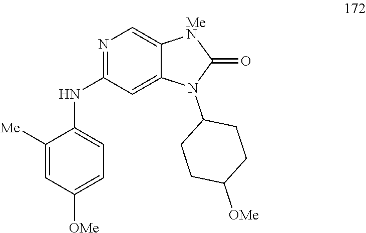 [SMILES: COc1ccc(Nc2cc3c(cn2)n(C)c(=O)n3C2CCC(OC)CC2)c(C)c1]

2-Chloro-N-(4-methoxycyclohexyl)-5-nitropyridin-4-amine (168). A solution of 4-methoxycyclohexan-1-amine hydrochloride (0.82 g, 4.95 mmol) and iPr₂NEt (0.99 mL, 5.66 mmol) in dry DCM (5 mL) was added dropwise to a stirred solution of nitropyridine 2 (0.91 g, 4.72 mmol) and iPr₂NEt (0.99 mL, 5.66 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (30-80%) of EtOAc/pet. ether, to give chloride 168 (1.21 g, 99%) as a yellow powder: mp 170-172° C.; ¹H NMR (CDCl₃) δ 9.02 (s, 1H, H-6), 8.16 (br d, J=6.8 Hz, 1H, 4-NH), 6.74 (s, 1H, H-3), 3.45-3.54 (m, 1H, H-4′), 3.38 (s, 3H, 4′-OCH₃), 3.21-3.30 (m, 1H, H-1′), 2.07-2.20 (m, 4H, 2×CH₂), 1.40-1.50 (m, 4H, 2×CH₂); MS m/z 286.0 (MH⁺, 100%), 288.0 (MH⁺, 35%).

6-Chloro-N⁴-(4-methoxycyclohexyl)pyridine-3,4-diamine (169). A solution of nitropyridine 168 (1.20 g, 4.66 mmol) in EtOAc (30 ml) was added dropwise to a stirred suspension of SnCl₂·2H₂O (4.20 g, 18.6 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 169 as a white foam: ¹H NMR (CDCl₃) δ 7.64 (s, 1H, H-2), 6.44 (s, 1H, H-5), 4.21 (br d, J=7.0 Hz, 1H, 4-NH), 3.37 (s, 3H, 4′-OCH₃), 3.24-3.30 (m, 1H, H-4′), 3.20 (tt, J=10.1, 3.3 Hz, 1H, H-1′), 2.95 (br s, 2H, 3-NH₂), 2.07-2.18 (m, 4H, 2×CH₂), 1.35-1.44 (m, 4H, 2×CH₂); MS m/z 256.0 (MH⁺, 100%), 258.1 (MH⁺, 35%).

6-Chloro-1-(4-methoxycyclohexyl)-1,3-dihydro-2H-imidazo[4,5-c]pyridin-2-one (170). CDI (0.76 g, 4.70 mmol) was added to a stirred solution of diamine 169 (1.02 g, 4.48 mmol) in dry MeCN (50 mL) at 20° C. The mixture was stirred at 20° C. for 24 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was precipitated from 20% EtOAc/pet. ether, to give the pyridinone 170 (0.99 g, 79%) as a white powder: mp 218-220° C.; ¹H NMR (CDCl₃) δ 9.33 (br s, 1H, 3-H), 8.11 (s, 1H, H-4), 7.07 (s, 1H, H-7), 4.24 (tt, J=12.3, 4.1 Hz, 1H, H-1′), 3.40 (s, 3H, 4′-OCH₃), 3.31 (tt, J=11.0, 4.1 Hz, 1H, H-4′), 2.27 (br d, J=12.9 Hz, 2 H, CH₂), 2.17 (br dt, J=13.0, 3.2 Hz, 2 H, CH₂), 1.93 (br d, J=12.6 Hz, 2 H, CH₂), 1.43 (ddd, J=13.0, 11.2, 3.2 Hz, 2 H, CH₂); MS m/z 282.0 (MH⁺, 100%), 284.0 (MH⁺, 35%).

6-Chloro-1-(4-methoxycyclohexyl)-3-methyl-1,3-dihydro-2H-imidazo[4,5-c]pyridin-2-one (171). NaH (60% dispersion, 167 mg, 4.18 mmol) was added to a stirred solution of pyridinone 170 (0.98 g, 3.48 mmol) and MeI (0.33 mL, 5.22 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (30-50%) of EtOAc/pet. ether, to give chloride 171 (0.68 g, 66%) as white needles: mp 140-143° C.; ¹H NMR (CDCl₃) δ 7.97 (s, 1H, H-4), 7.03 (d, J=0.4 Hz, 1H, H-7), 4.23 (tt, J=12.4, 4.1 Hz, 1H, H-1′), 3.43 (s, 3H, 3-CH₃), 3.40 (s, 3H, 4′-OCH₃), 3.29 (tt, J=11.0, 4.1 Hz, 1H, H-4′), 2.22 (m, 2H, CH₂), 2.15 (ddd, J=13.2, 12.7, 3.5 Hz, 2 H, CH₂), 1.86-1.94 (m, 2H, CH₂), 1.62 (ddd, J=13.1, 11.2, 3.5 Hz, 2 H, CH₂); MS m/z 268.0 (MH⁺, 100%), 270.0 (MH⁺, 35%).

6-((4-Methoxy-2-methylphenyl)amino)-1-(4-methoxycyclohexyl)-3-methyl-1,3-dihydro-2H-imidazo[4,5-c]pyridin-2-one (172). A degassed mixture of chloride 171 (136 mg, 0.46 mmol), aniline (76 mg, 0.55 mmol), Pd₂dba₃ (21 mg, 23 μmol), XPhos (44 mg, 92 μmol) and Cs₂CO₃ (330 mg, 1.01 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 172 (96 mg, 53%) as tan needles: mp (EtOAc/pet ether) 146-148° C.; ¹H NMR (CDCl₃) δ 7.72 (s, 1H, H-4), 7.22 (d, J=8.6 Hz, 1H, H-6″), 6.82 (d, J=2.9 Hz, 1H, H-3″), 6.76 (dd, J=8.6, 2.9 Hz, 1H, H-5″), 6.11 (d, J=0.5 Hz, 1H, H-7), 5.92 (s, 1H, 6-NH), 4.01 (tt, J=12.2, 4.0 Hz, 1H, H-4′), 3.83 (s, 3H, 4″-OCH₃), 3.34 (s, 3H, 4′-OCH₃), 3.32 (s, 3H, 3-CH₃), 3.15 (tt, J=11.0, 4.0 Hz, 1H, H-1′), 2.23 (s, 3H, 2″-CH₃), 2.17 (br d, J=12.7 Hz, 2 H, CH₂), 2.09 (dq, J=12.8, 3.4 Hz, 2 H, CH₂), 1.82 (br d, J=12.8 Hz, 2 H, CH₂), 1.38-1.40 (m, 2H, CH₂); MS m/z 397.2 (MH⁺, 100%); HRMS calcd for C₂₂H₂₉N₄O₃ (MH⁺) m/z 397.2234, found 397.2237 (−0.6 ppm). HPLC purity 99.7%.

### Example 121: SN39539 6-((4-Chloro-2-methylphenyl)amino)-1-(4-methoxycyclohexyl)-3-methyl-1,3-dihydro-2H-imidazo[4,5-c]pyridin-2-one (173)

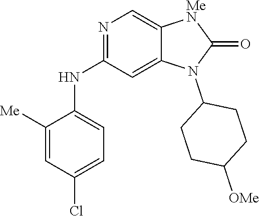 [SMILES: COC1CCC(n2c(=O)n(C)c3cnc(Nc4ccc(Cl)cc4C)cc32)CC1]

A degassed mixture of chloride 171 (128 mg, 0.43 mmol), 4-chloro-2-methylaniline (74 mg, 0.52 mmol), Pd₂dba₃ (22 mg, 20 μmol), XPhos (41 mg, 86 μmol) and Cs₂CO₃ (300 mg, 0.95 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (EtOAc) gave imidazopyridinone 173 (121 mg, 70%) as red needles: mp (EtOAc/pet ether) 181-183° C.; 1H NMR (CDCl₃) δ 7.81 (s, 1H, H-4), 7.39 (d, J=8.6 Hz, 1H, H-6″), 7.23 (d, J=2.4 Hz, 1H, H-3″), 7.17 (dd, J=8.6, 2.4 Hz, 1H, H-5″), 6.42 (s, 1H, H-7), 6.02 (s, 1H, 6-NH), 4.12 (tt, J=12.3, 4.0 Hz, 1H, H-4′), 3.38(s, 3 H, 4′-OCH₃), 3.35 (s, 3H, 3-CH₃), 3.20 (tt, J=12.3, 4.0 Hz, 1 H, H-1′), 2.27 (s, 3H, 2″-CH₃), 2.06-2.22 (m, 4H, 2×CH₂), 1.86 (br d, J=13.0 Hz, 2 H, CH₂), 1.38 (ddd, J=13.0, 11.2, 3.4 Hz, 2 H, CH₂); MS m/z 401.2 (MH⁺, 100%); HRMS calcd for C₂₁H₂₆ClN₄O₂ (MH⁺) m/z 401.1739, found 401.1736 (0.7 ppm). HPLC purity 95.2%.

### Example 122: SN39581 1-(4-(Benzyloxy)cyclohexyl)-6-((4-methoxy-2-methylphenyl)amino)-3-methyl-1,3-dihydro-2H-imidazo[4,5-c]pyridin-2-one (178)

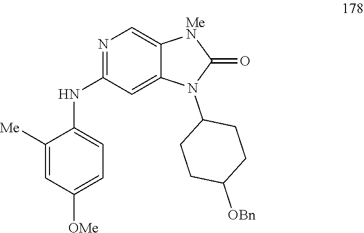 [SMILES: COc1ccc(Nc2cc3c(cn2)n(C)c(=O)n3C2CCC(OCc3ccccc3)CC2)c(C)c1]

N-(4-(Benzyloxy)cyclohexyl)-2-chloro-5-nitropyridin-4-amine(174). iPr₂NEt (1.87 mL, 10.7 mmol) was added to a stirred suspension of nitropyridine 2 (0.94 g, 4.88 mmol) and 4-(benzyloxy)cyclohexan-1-amine hydrochloride (1.24 g, 4.95 mmol) in dry DCM (50 mL) at 5° C. The mixture was stirred at 20° C. for 16 h before being diluted with DCM (100 mL) and washed with water (3×50 mL), dried (MgSO₄) and the solvent evaporated. The residue was purified by chromatography, eluting with a gradient (10-20%) of EtOAc/pet. ether, to give chloride 174 (1.36 g, 77%) as yellow crystals: mp 131-133° C.; ¹H NMR (CDCl₃) δ 9.01 (s, 1H, H-6), 8.15 (br d, J=7.4 Hz, 1H, 4-NH), 7.34-7.38 (m, 4H, H-2″, H-3″, H-5″, H-6″), 7.27-7.32 (m, 1H, H-4″), 6.73 (s, 1H, H-3), 4.58 (s, 2H, CH₂O), 3.44-3.54 (m, 2H, H-1′, H-4′), 2.12-2.19 (m, 4H, 2×CH₂), 1.52-1.60 (m, 2H, CH₂), 1.43-1.48 (m, 2H, CH₂); MS m/z 262.1 (MH⁺, 100%), 262.1 (MH⁺, 35%); HRMS calcd for C₁₈H₂₁³⁵ClN₃O₃(MH⁺) m/z 362.1266, found 362.1275 (−2.5 ppm).

N⁴-(4-(Benzyloxy)cyclohexyl)-6-chloropyridine-3,4-diamine (175). A solution of nitropyridine 174 (1.28 g, 3.55 mmol) in EtOAc (30 ml) was added dropwise to a stirred suspension of SnCl₂·2H₂O (3.20 g, 14.2 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated. The residue was purified by chromatography, eluting with EtOAc, to give diamine 175 as a white powder: mp 164-166° C.; ¹H NMR (CDCl₃) δ 7.64 (s, 1H, H-2), 7.33-7.38 (m, 4H, H-2″, H-3″, H-5″, H-6″), 7.27-7.31 (m, 1H, H-4″), 6.44 (s, 1H, H-5), 4.58 (s, 2H, CH₂O), 4.20 (br d, J=7.2 Hz, 1H, 4-NH), 3.37-3.46 (m, 1H, H-1′), 3.23-3.33 (m, 1H, H-4′), 2.93 (br s, 2H, 3-NH₂), 2.10-2.19 (m, 4H, 2×CH₂), 1.44-1.56 (m, 2H, CH₂), 1.22-1.31 (m, 2H, CH₂); MS m/z 332.1 (MH⁺, 100%), 334.1 (MH⁺, 35%); HRMS calcd for C₁₈H₂₃₃₅ClN₃O (MH⁺) m/z 332.1524, found 332.1535 (−3.3 ppm).

1-(4-(Benzyloxy)cyclohexyl)-6-chloro-1,3-dihydro-2H-imidazo[4,5-c]pyridin-2-one (176). CDI (0.55 g, 3.42 mmol) was added to a stirred solution of diamine 175 (1.03 g, 3.11 mmol) in dry MeCN (50 mL) at 20° C. The mixture was stirred at 20° C. for 24 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was precipitated from 20% EtOAc/pet. ether, to give the pyridinone 176 (0.75 g, 67%) as a white powder: mp 228-231° C.; ¹H NMR (CDCl₃) δ 9.46 (br s, 1H, 3-H), 8.11 (s, 1H, H-4), 7.36-7.40 (m, 4H, H-2″, H-3″, H-5″, H-6″), 7.28-7.33 (m, 1H, H-4″), 7.05 (s, 1H, H-7), 4.58 (s, 2H, CH₂O), 4.25 (tt, J=12.4, 4.0 Hz, 1H, H-1′), 3.51 (tt, J=11.0, 4.1 Hz, 1H, H-4′), 2.30 (br d, J=12.3 Hz, 2 H, CH₂), 2.25 (ddd, J=13.1, 12.9, 3.2 Hz, 2 H, CH₂), 1.93 (br d, J=11.9 Hz, 2 H, CH₂), 1.54 (ddd, J=13.1, 11.2, 3.2 Hz, 2 H, CH₂); MS m/z 358.0 (MH⁺, 100%), 360.0 (MH⁺, 35%); HRMS calcd for C₁₉H₂₁³⁵ClN₃O₂ (MH⁺) m/z 358.1317, found 358.1327 (−2.9 ppm).

1-(4-(Benzyloxy)cyclohexyl)-6-chloro-3-methyl-1,3-dihydro-2H-imidazo[4,5-c]pyridin-2-one (177). NaH (60% dispersion, 96 mg, 2.40 mmol) was added to a stirred solution of pyridinone 176 (0.72 g, 2.00 mmol) and MeI (0.19 mL, 3.00 mmol) in dry DMF (10 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with 40% EtOAc/pet. ether, to give chloride 177 (0.68 g, 92%) as a white powder: mp 176-178° C.; ¹H NMR (CDCl₃) δ 7.97 (s, 1H, H-4), 7.34-7.39 (m, 4H, H-2″, H-3″, H-5″, H-6″), 7.28-7.32 (m, 1H, H-4″), 7.01 (d, J=0.4 Hz, 1H, H-7), 4.60 (s, 2H, CH₂O), 4.24 (tt, J=12.5, 4.1 Hz, 1H, H-1′), 3.49 (tt, J=11.0, 4.1 Hz, 1H, H-4′), 3.42 (s, 3H, 3-CH₃), 2.27 (br d, J=12.9 Hz, 2 H, CH₂), 2.15 (ddd, J=13.2, 12.7, 3.4 Hz, 2 H, CH₂), 1.89 (br d, J=12.7 Hz, 2 H, CH₂), 1.53 (ddd, J=13.1, 11.2, 3.4 Hz, 2 H, CH₂); MS m/z 372.1 (MH⁺, 100%), 374.1 (MH⁺, 35%); HRMS calcd for C₂₀H₂₃³⁵ClN₃O₂ (MH⁺) m/z 372.1473, found 372.1483 (−2.7 ppm).

1-(4-(Benzyloxy)cyclohexyl)-6-((4-methoxy-2-methylphenyl)amino)-3-methyl-1,3-dihydro-2H-imidazo[4,5-c]pyridin-2-one (178). A degassed mixture of chloride 177 (212 mg, 0.57 mmol), 4-methoxy-2-methylaniline (94 mg, 0.68 mmol), Pd₂dba₃ (26 mg, 29 μmol), XPhos (54 mg, 114 μmol) and Cs₂CO₃ (409 mg, 1.25 mmol) in MeCN (10 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 178 (188 mg, 70%) as cream needles: mp (EtOAc/pet ether) 154-156° C.; ¹H NMR (CDCl₃) δ 7.75 (s, 1H, H-4), 7.32-7.38 (m, 4H, H-2″′, H-3″′, H-5″′, H-6″′), 7.26-7.30 (m, 1H, H-4″′), 7.23 (d, J=8.6 Hz, 1H, H-6′), 6.83 (d, J=2.9 Hz, 1H, H-3′), 6.78 (dd, J=8.6, 2.9 Hz, 1H, H-5′), 6.14 (d, J=0.4 Hz, 1H, H-7), 5.93 (s, 1H, 6-NH), 4.56 (s, 2H, CH₂O), 4.05 (tt, J=12.3, 4.0 Hz, 1H, H-1″), 3.82 (s, 3H, 4′-OCH₃), 3.33-3.40 (m, 4H, H-4″, 3-CH₃), 2.25 (s, 3H, 2′-CH₃), 2.20 (br d, J=12.2 Hz, 2 H, CH₂), 2.10 (ddd, J=13.1, 12.8, 3.4 Hz, 2 H, CH₂), 1.81 (br d, J=12.4 Hz, 2 H, CH₂), 1.45 (ddd, J=13.1, 11.2, 3.4 Hz, 2 H, CH₂); MS m/z 473.2 (MH⁺, 100%); HRMS calcd for C₂₈H₃₃³⁵ClN₄O₃ (MH⁺) m/z 473.2547, found 473.2563 (−3.4 ppm). HPLC purity 99.6%.

### Example 123: SN39584 1-(4-Hydroxycyclohexyl)-6-((4-methoxy-2-methylphenyl)amino)-3-methyl-1,3-dihydro-2H-imidazo[4,5-c]pyridin-2-one (179)

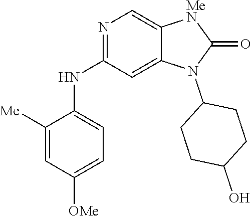 [SMILES: COc1ccc(Nc2cc3c(cn2)n(C)c(=O)n3C2CCC(O)CC2)c(C)c1]

A mixture of benzyl ether 178 (129 mg, 0.27 mmol) and Pd/C (20 mg) in EtOH/EtOAc (2:1, 50 mL) was stirred under H₂ (50 psi) for 16 h. The mixture was filtered through diatomaceous earth and the pad washed with EtOH (25 mL). The solvent was evaporated and the residue purified by chromatography, eluting with a gradient (0-5%) of MeOH/DCM, to give imidazopyridinone 179 (62 mg, 60%) as white crystals: mp 209-211° C.; ¹H NMR (CDCl₃) δ 7.75 (s, 1H, H-4), 7.23 (d, J=8.6 Hz, 1H, H-6′), 6.84 (d, J=2.9 Hz, 1H, H-3′), 6.78 (dd, J=8.6, 2.9 Hz, 1H, H-5′), 6.13 (d, J=0.5 Hz, 1H, H-7), 5.99 (br s, 1H, 6-NH), 4.06 (tt, J=12.2, 4.0 Hz, 1H, H-1″), 3.83 (s, 3H, 4′-OCH₃), 3.63 (tt, J=11.0, 4.0 Hz, 1H, H-4″), 3.36 (s, 3H, 3-CH₃), 2.25 (s, 3H, 2′-CH₃), 2.03-2.17 (m, 4H, 2×CH₂), 1.79 (br d, J=11.0 Hz, 2 H, CH₂), 1.38-1.49 (m, 2H, CH₂), OH not observed; MS m/z 383.2 (MH⁺, 100%); HRMS calcd for C₂₁H₂₇N₄O₃ (MH⁺) m/z 383.2078, found 383.2085 (−1.9 ppm). HPLC purity 99.0%.

### Example 124: SN40297 tert-Butyl (4-(6-((4-Methoxy-2-methylphenyl)amino)-3-methyl-2-oxo-2,3-dihydro-1H-imidazo[4,5-c]pyridin-1-yl)cyclohexyl)carbamate (184)

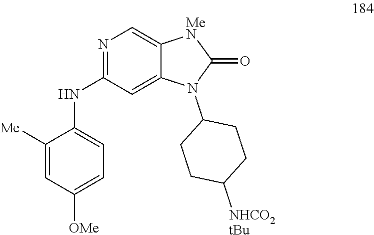

tert-Butyl (4-((2-Chloro-5-nitropyridin-4-yl)amino)cyclohexyl)carbamate (180). iPr₂NEt (1.41 mL, 8.09 mmol) was added dropwise to a stirred solution of nitropyridine 2 (1.20 g, 6.22 mmol) and tert-butyl (4-aminocyclohexyl)carbamate (1.47 g, 6.84 mmol) in dry dioxane (50 mL) at 5° C. The mixture was stirred at 20° C. for 16 h before being diluted with EtOAc (150 mL) and washed with water (3×50 mL), dried (MgSO₄) and the solvent evaporated. The residue was purified by chromatography, eluting with a gradient (20-50%) of EtOAc/pet. ether, to give chloride 180 (1.48 g, 64%) as a yellow powder: mp 199-201° C.; 1H NMR (CDCl₃) δ 9.04 (s, 1H, H-6″), 8.10 (br d, J=7.5 Hz, 1H, 4′-NH), 6.73 (s, 1H, H-3″), 4.35-4.50 (m, 1H, 1′-NH), 3.48-3.60 (m, 1H, H-1′), 3.38-3.48 (m, 1H, H-4′), 2.19 and 2.04 (2×br d, J=12.3 Hz, 4 H, 2×CH₂), 1.48-1.58 (m, 2H, CH₂), 1.47 and 1.45 (2×s, 9 H, CO₂tBu), 1.33 and 1.22 (2 br q, J=11.4 Hz, 2 H, CH₂); MS m/z 271.1 (MH⁺, 100%), 273.1 (MH⁺, 35%); HRMS calcd for C₁₆H₂₄³⁵ClN₄O₄ (MH⁺) m/z 371.1494, found 371.1490 (−1.0 ppm).

tert-Butyl (4-((5-Amino-2-chloropyridin-4-yl)amino)cyclohexyl)carbamate (181). A mixture of nitropyridine 180 (1.44 g, 3.87 mmol), Zn powder (2.53 g, 38.7 mmol) and NH₄Cl (2.07 g, 38.7 mmol) in MeOH/THF (1:1, 100 mL) at 20° C. for 2 h. The mixture was filtered through a pad of diatomaceous earth and washed with EtOAc (40 mL). The combined organic fraction was dried (MgSO₄), filtered and the solvent evaporated to give diamine 181 (1.32 g, 100%) as a red gum: ¹H NMR (CDCl₃) δ 7.74 (s, 1H, H-6″), 6.42 (s, 1H, H-3″), 4.30-4.45 (m, 2H, 1′-NH, 4′-NH), 3.43-3.53 (m, 2H, 5″-NH₂), 3.18-3.25 (m, 2H, H-1′, H-4′), 2.07-2.17 (m, 4H, 2×CH₂), 1.53-1.62 (m, 2H, CH₂), 1.43 and 1.45 (2×s, 9 H, CO₂tBu), 1.28-1.38 and 1.16-1.26 (2×m, 2 H, CH₂); MS m/z 341.1 (MH⁺, 100%), 343.1 (MH⁺, 35%); HRMS calcd for C₁₆H₂₆³⁵ClN₄O₂ (MH⁺) m/z 341.1739, found 341.1745 (−1.9 ppm).

tert-Butyl (4-(6-Chloro-2-oxo-2,3-dihydro-1H-imidazo[4,5-c]pyridin-1-yl)cyclohexyl)carbamate (182). CDI (0.75 g, 4.64 mmol) was added to a stirred solution of diamine 181 (1.32 g, 3.87 mmol) in dry MeCN (50 mL) at 20° C. The mixture was stirred at 20° C. for 96 h. The solvent was evaporated and the residue partitioned between CHCl₃ (150 mL) and water (100 mL). The organic fraction was washed with water (2×50 mL), washed with brine (50 mL), dried (MgSO₄), filtered and the solvent evaporated. The residue was purified by chromatography, eluting with a gradient (60-100%) of EtOAc/pet. ether, to give pyridinone 182 (1.23 g, 86%) as a white powder: mp 244-247° C.; ¹H NMR (CDCl₃) δ 11.32 (br s, 1H, 3″-H), 7.94 (s, 1H, H-4″), 7.61 (s, 1H, H-7), 6.77 (d, J=7.9 Hz, 1H, 1′-NH), 4.12 (tt, J=12.4, 3.8 Hz, 1H, H-4′), 3.40-3.48 (m, 1H, H-1′), 2.18 (dq, J=12.7, 2.8 Hz, 2 H, CH₂), 1.87 (br d, J=11.0 Hz, 2 H, CH₂), 1.66 (br d, J=10.8 Hz, 2 H, CH₂), 1.28-1.40 (m, 11H, CO₂tBu, CH₂); MS m/z 367.1 (MH⁺, 100%), 369.1 (MH⁺, 35%); HRMS calcd for C₁₇H₂₄³⁵ClN₄O₃ (MH⁺) m/z 367.1531, found 367.1534 (−0.6 ppm).

tert-Butyl (4-(6-chloro-3-methyl-2-oxo-2,3-dihydro-1H-imidazo[4,5-c]pyridin-1-yl)cyclohexyl)carbamate (183). NaH (60% dispersion, 161 mg, 4.02 mmol) was added to a stirred solution of pyridinone 182 (1.23 g, 3.35 mmol) and MeI (0.32 mL, 5.03 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was purified by chromatography, eluting with a gradient (50-100%) of EtOAc/pet. ether, to give chloride 183 (0.92 g, 72%) as a white powder: mp 247-249° C.; ¹H NMR (CDCl₃) δ 7.97 (s, 1H, H-4″), 7.04 (d, J=0.4 Hz, 1H, H-7), 4.35-4.50 (m, 1H, H-1′), 4.20-4.30 (m, 1H, H-4′) 3.35-3.63 (m, 4H, 1′-NH, 3″-CH₃), 2.14-2.26 (m, 4H, 2×CH₂), 1.98-2.03 and 1.85-1.94 (2×m, 2 H, CH₂), 1.43 and 1.46 (2×s, 9 H, CO₂tBu), 1.15-1.23 and 1.30-1.39 (2×m, 2 H, CH₂); MS m/z 381.0 (MH⁺, 100%), 383.0 (MH⁺, 35%); HRMS calcd for C₁₈H₂₆³⁵ClN₄O₃ (MH⁺) m/z 381.1688, found 381.1697 (−2.3 ppm).

tert-Butyl (4-(6-((4-Methoxy-2-methylphenyl)amino)-3-methyl-2-oxo-2,3-dihydro-1H-imidazo[4,5-c]pyridin-1-yl)cyclohexyl)carbamate (184). A degassed mixture of chloride 183 (235 mg, 0.49 mmol), 4-methoxy-2-methylaniline (80 mg, 0.59 mmol), Pd₂dba₃ (22 mg, 25 μmol), XPhos (47 mg, 98 μmol) and Cs₂CO₃ (351 mg, 1.08 mmol) in MeCN (8 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 184 (99 mg, 33%) as a white powder: ¹H NMR (CDCl₃) δ 7.75 (s, 1H, H-4″), 7.25 (d, J=8.6 Hz, 1H, H-6″′), 6.85 (d, J=2.9 Hz, 1H, H-3″′), 6.79 (dd, J=8.6, 2.9 Hz, 1H, H-5), 6.16 (s, 1H, H-7), 5.90 (br s, 1H, 6″-NH), 4.35-4.41 (m, 1H, H-1′), 4.08-4.18 (m, 1H, H-4′), 3.84 (s, 3H, 4″′—OCH₃), 3.36-3.48 (m, 4H, 1′-NH, 3″-CH₃), 2.26 (s, 3H, 2″-CH₃), 2.03-2.15 (m, 4H, 2×CH₂), 1.76-1.85 (m, 2H, CH₂), 1.44 (s, 9H, CO₂tBu), 1.31-1.42 (m, 2H, CH₂); MS m/z 482.2 (MH⁺, 100%); HRMS calcd for C₂₆H₃₆N₅₀₄ (MH⁺) m/z 482.2762, found 482.2770 (−1.6 ppm).

### Example 125: SN39695 1-(4-Aminocyclohexyl)-6-((4-methoxy-2-methylphenyl)amino)-3-methyl-1,3-dihydro-2H-imidazo[4,5-c]pyridin-2-one (185)

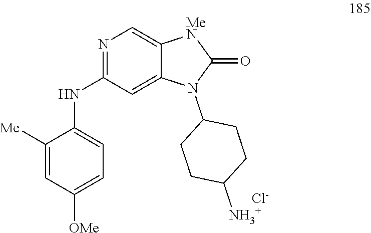 [SMILES: COc1ccc(Nc2cc3c(cn2)n(C)c(=O)n3C2CCC([NH3+])CC2)c(C)c1 ; [Cl-]]

A mixture of carbamate 184 (84 mg, 0.17 mmol) and 4 M HCl in dioxane (1.0 mL, 4 mmol) in MeOH (5 mL) was stirred at 20° C. for 16 h. The solvent was evaporated and the residue triturated with EtOAc (10 mL) and dried to give imidazopyridinone 185 (74 mg, 100%) as a white powder: ¹H NMR [(CD₃)₂SO] δ 9.83 (br s, 1H, 6-NH), 8.25 (br s, 3H, NH₂·HCl), 7.76 (s, 1H, H-4), 7.23 (d, J=8.7 Hz, 1H, H-6′), 7.08 (s, 1H, H-7), 6.98 (d, J=2.9 Hz, 1H, H-3′), 6.89 (dd, J=8.7, 2.9 Hz, 1H, H-5′), 4.07-4.13 (m, 1H, H-1″), 3.80 (s, 3H, 4′-OCH₃), 3.28 (s, 3H, 3-CH₃), 3.00-3.08 (m, 1H, H-4″), 2.20 (s, 3H, 2″-CH₃), 2.05-2.16 (m, 4H, 2×CH₂), 1.80-1.88 (m, 2H, CH₂), 1.50-1.62 (m, 2H, CH₂); MS m/z 382.2 (MH⁺, 100%); HRMS calcd for C₂₁H₂₈N₅O₂ (MH⁺) m/z 382.2238, found 382.2241 (−0.9 ppm). HPLC purity 98.3%.

### Example 126: SN39623 6-((4-Methoxy-2-methylphenyl)amino)-3-methyl-1-phenyl-1,3-dihydro-2H-imidazo[4,5-c]pyridin-2-one (190)

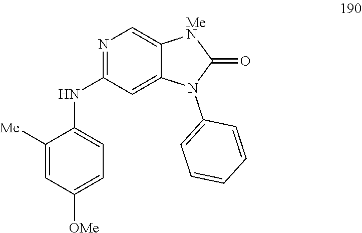 [SMILES: COc1ccc(Nc2cc3c(cn2)n(C)c(=O)n3-c2ccccc2)c(C)c1]

2-Chloro-5-nitro-N-phenylpyridin-4-amine (186). A solution of aniline (0.35 mL, 3.82 mmol) in dry DCM (10 mL) was added to a stirred solution of nitropyridine 2 (0.67 g, 3.47 mmol) and iPr₂NEt (0.91 mL, 5.21 mmol) in dry DCM (50 mL) at 20° C. The mixture was stirred at 20° C. for 16 h before being diluted with DCM (100 mL) and washed with water (3×100 mL), dried (MgSO₄) and the solvent evaporated. The residue was purified by chromatography, eluting with 10% EtOAc/pet. ether, to give chloride 186 (787 mg, 91%) as yellow plates: mp 149-150° C.; ¹H NMR (CDCl₃) δ 9.66 (br s, 1H, 4-NH), 9.10 (s, 1H, H-6), 7.49-7.53 (m, 2H, H-3′, H-5′), 7.39 (br t, J=7.5 Hz, 1H, H-4′), 7.29 (br d, J=7.4 Hz, 2 H, H-2′, H-6′), 6.92 (s, 1H, H-3); MS m/z 250.0 (MH⁺, 100%), 252.1 (MH⁺, 35%); HRMS calcd for C₁₁H₉³⁵ClN₃O₂ (MH⁺) m/z 250.0378, found 250.0372 (2.5 ppm).

6-Chloro-N⁴-phenylpyridine-3,4-diamine (187). A mixture of nitropyridine 186 (0.75 g, 3.00 mmol), Zn powder (1.96 g, 30.0 mmol) and NH₄Cl (1.60 g, 30.0 mmol) in MeOH/THF (1:1, 50 mL) at 20° C. for 2 h. The mixture was filtered through a pad of diatomaceous earth and washed with EtOAc (40 mL). The combined organic fraction was dried (MgSO₄), filtered and the solvent evaporated. The residue was purified by chromatography, eluting with a gradient (30-100%) of EtOAc/pet. ether, to give diamine 187 as a purple oil: ¹H NMR (CDCl₃) δ 7.80 (s, 1H, H-6), 7.38 (br dd, J=8.9, 7.0 Hz, 2 H, H-3′, H-5′), 7.10-7.17 (m, 3H, H-3′, H-4′, H-5′), 6.96 (s, 1H, H-3), 6.00 (br s, 1H, 4-NH), 3.26 (br s, 2H, 5-NH₂); MS m/z 220.1 (MH⁺, 100%), 222.2 (MH⁺, 35%); HRMS calcd for C₁₁H₁₁³⁵ClN₃ (MH⁺) m/z calcd 220.0636, found 220.0633 (1.5 ppm).

6-Chloro-1-phenyl-1,3-dihydro-2H-imidazo[4,5-c]pyridin-2-one (188). CDI (0.27 g, 1.65 mmol) was added to a stirred solution of diamine 187 (0.33 g, 1.50 mmol) in dry MeCN (30 mL) at 20° C. The mixture was stirred at 20° C. for 48 h. The solvent was evaporated and the residue partitioned between CHCl₃ (200 mL) and water (100 mL). The organic fraction was washed with water (2×50 mL), washed with brine (50 mL), dried (MgSO₄), filtered and the solvent evaporated. The residue was precipitated from 20% EtOAc/pet. ether, to give the pyridinone 188 (0.20 g, 55%) as pink needles: mp 284-288° C.; ¹H NMR [(CD₃)₂SO] δ 11.64 (br s, 1H, 3-H), 8.07 (s, 1H, H-4), 7.53-7.61 (m, 4H, H-2′, H-3′, H-5′, H-6′), 7.44-7.50 (m, 1H, H-4′), 6.74 (s, 1H, H-7); MS m/z 246.1 (MH⁺, 100%), 248.1 (MH⁺, 35%); HRMS calcd for C₁₂H₉₃₅ClN₃O (MH⁺) m/z calcd 246.0429, found 246.0432 (−1.5 ppm).

6-Chloro-3-methyl-1-phenyl-1,3-dihydro-2H-imidazo[4,5-c]pyridin-2-one (189). NaH (60% dispersion, 37 mg, 0.92 mmol) was added to a stirred solution of pyridinone 188 (188 mg, 0.77 mmol) and MeI (72 μL, 1.12 mmol) in dry DMF (5 mL) at 5° C. The mixture was stirred at 20° C. for 16 h and then quenched with ice/water (5 mL). The solvent was evaporated and the residue was partitioned between EtOAc (50 mL) and water (50 ml). The organic fraction was washed with water (2×50 mL), washed with brine (50 mL), dried (MgSO₄), filtered and the solvent evaporated. The residue was purified by chromatography, eluting with 50% EtOAc/pet. ether, to give chloride 189 (152 mg, 76%) as white powder: mp 170-172° C.; ¹H NMR (CDCl₃) δ 8.07 (s, 1H, H-4), 7.54-7.60 (m, 2H, H-2′, H-6′), 7.44-7.59 (m, 3H, H-3′, H-4′, H-5′), 7.02 (d, J=0.4 Hz, 1H, H-7), 3.32 (s, 3H, 3-CH₃); MS m/z 260.1 (MH⁺, 100%), 262.1 (MH⁺, 35%); HRMS calcd for C₁₃H₁₁³⁵ClN₃O (MH⁺) m/z calcd 260.0585, found 260.0589 (−1.5 ppm).

6-((4-Methoxy-2-methylphenyl)amino)-3-methyl-1-phenyl-1,3-dihydro-2H-imidazo[4,5-c]pyridin-2-one (190). A degassed mixture of chloride 189 (120 mg, 0.46 mmol), 4-methoxy-2-methylaniline (76 mg, 0.55 mmol), Pd₂dba₃ (21 mg, 23 μmol), XPhos (44 mg, 92 μmol) and Cs₂CO₃ (330 mg, 1.01 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 190 (80 mg, 48%) as a tan solid: mp 70-73° C.; ¹H NMR (CDCl₃) δ 7.84 (d, J=0.5 Hz, 1H, H-4), 7.46-7.50 (m, 2H, H-3′, H-5′), 7.33-7.42 (m, 3H, H-2′, H-4′, H-6′), 7.18 (d, J=8.6 Hz, 1H, H-6″), 6.77 (d, J=2.9 Hz, 1H, H-3″), 6.70 (dd, J=8.6, 2.9 Hz, 1 H, H-5″), 6.15 (d, J=0.6 Hz, 1H, H-7), 5.99 (br s, 1H, 6-NH), 3.78 (s, 3H, 4″-OCH₃), 3.46 (s, 3H, 3-CH₃), 2.22 (s, 3H, 2″-CH₃); MS m/z 361.2 (MH⁺, 100%); HRMS calcd for C₂₁H₂₁N₄O₂ (MH⁺) m/z calcd 361.1659, found 361.1664 (−1.6 ppm). HPLC purity 97.0%.

### Example 127: SN39519 6-((4-Methoxy-2-methylphenyl)amino)-1-(4-methoxyphenyl)-3-methyl-1,3-dihydro-2H-imidazo[4,5-c]pyridin-2-one (195)

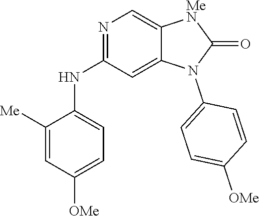 [SMILES: COc1ccc(-n2c(=O)n(C)c3cnc(Nc4ccc(OC)cc4C)cc32)cc1]

2-Chloro-N-(4-methoxyphenyl)-5-nitropyridin-4-amine (191). A solution of anisidine (1.46 g, 11.8 mmol) in dry DCM (10 mL) was added to a stirred solution of nitropyridine 2 (2.17 g, 11.3 mmol) and iPr₂NEt (2.36 mL, 13.5 mmol) in dry DCM (100 mL) at 20° C. The mixture was stirred at 20° C. for 16 h before being diluted with DCM (100 mL) and washed with water (3×100 mL), dried (MgSO₄) and the solvent evaporated. The residue was purified by chromatography, eluting with 10% EtOAc/pet. ether, to give chloride 191 (2.90 g, 92%) as yellow plates: mp 137-140° C.; ¹H NMR (CDCl₃) δ 9.52 (br s, 1H, 4-NH), 9.08 (s, 1H, H-6), 7.19 (ddd, J=8.8, 3.3, 2.2 Hz, 2 H, H-2′, H-6′), 7.01 (ddd, J=8.8, 3.3, 2.2 Hz, 2 H, H-3′, H-5′), 6.77 (s, 1H, H-3), 3.87 (s, 3H, 4′-OCH₃); MS m/z 280.0 (MH⁺, 100%), 282.1 (MH⁺, 35%). Analysis calcd for C₁₂H₁₀ClN₃O₃: C, 51.53; H, 3.60; N, 15.02. Found: C, 51.68; H, 3.72; N, 15.23%.

6-Chloro-N⁴-(4-methoxyphenyl)pyridine-3,4-diamine (192). A solution of nitropyridine 191 (2.90 g, 10.4 mmol) in EtOAc (50 ml) was added drop wise to a stirred suspension of SnCl₂·2H₂O (9.36 g, 41.48 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 192 as white needles: mp 129-131° C.; 1H NMR (CDCl₃) δ 7.75 (s, 1H, H-2), 7.10 (ddd, J=8.9, 3.4, 2.0 Hz, 2 H, H-2′, H-6′), 6.93 (ddd, J=8.9, 3.4, 2.0 Hz, 2 H, H-3′, H-5′), 6.69 (s, 1H, H-5), 5.92 (br s, 1H, 4-NH), 3.83 (s, 3H, 4′-OCH₃), 3.20 (br s, 2H, 5-NH₂); MS m/z 250.1 (MH⁺, 100%), 252.2 (MH⁺, 35%).

6-Chloro-1-(4-methoxyphenyl)-1,3-dihydro-2H-imidazo[4,5-c]pyridin-2-one (193). CDI (1.66 g, 10.2 mmol) was added to a stirred solution of diamine 192 (2.42 g, 9.72 mmol) in dry MeCN (80 mL) at 20° C. The mixture was stirred at 20° C. for 48 h. The solvent was evaporated and the residue partitioned between CHCl₃ (200 mL) and water (100 mL). The organic fraction was washed with water (2×50 mL), washed with brine (50 mL), dried (MgSO₄), filtered and the solvent evaporated. The residue was precipitated from 20% EtOAc/pet. ether, to give the pyridinone 193 (2.29 g, 85%) as a white powder: mp 291-293° C.; ¹H NMR [(CD₃)₂SO] δ 11.56 (br s, 1H, 3-H), 8.05 (s, 1H, H-4), 7.45 (ddd, J=9.0, 3.4, 2.2 Hz, 2 H, H-2′, H-6′), 7.12 (ddd, J=9.0, 3.4, 2.2 Hz, 2 H, H-3′, H-5′), 6.45 (s, 1H, H-7), 3.82 (s, 3H, 4′-OCH₃); MS m/z 276.1 (MH⁺, 100%), 278.1 (MH⁺, 35%). Analysis calcd for C₁₃H₁₀ClN₃O₂: C, 56.64; H, 3.66; N, 15.24. Found: C, 56.77; H, 3.66; N, 15.37%.

6-Chloro-1-(4-methoxyphenyl)-3-methyl-1,3-dihydro-2H-imidazo[4,5-c]pyridin-2-one (194). NaH (60% dispersion, 360 mg, 9.06 mmol) was added to a stirred solution of pyridinone 193 (2.27 g, 8.23 mmol) and MeI (0.67 mL, 10.7 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was triturated with pet. ether and dried to give chloride 194 (2.08 g, 87%) as white powder: mp 176-178° C.; ¹H NMR (CDCl₃) δ 8.05 (s, 1H, H-4), 7.36 (ddd, J=9.0, 3.4, 2.2 Hz, 2 H, H-2′, H-6′), 7.06 (ddd, J=9.0, 3.4, 2.2 Hz, 2 H, H-3′, H-5′), 6.94 (s, 1H, H-7), 3.88 (s, 3H, 4′-OCH₃), 3.52 (s, 3H, 3-CH₃); MS m/z 290.1 (MH⁺, 100%), 292.1 (MH⁺, 35%). Anal. calcd for C₁₄H₁₂ClN₃O₂·0.15EtOAc: C, 57.89; H, 4.39; N, 13.87. Found: C, 57.81; H, 4.03; N, 13.49%.

6-((4-Methoxy-2-methylphenyl)amino)-1-(4-methoxyphenyl)-3-methyl-1,3-dihydro-2H-imidazo[4,5-c]pyridin-2-one (195). A degassed mixture of chloride 194 (129 mg, 0.45 mmol), 4-methoxy-2-methylaniline (73 mg, 0.53 mmol), Pd₂dba₃ (23 mg, 20 μmol), XPhos (43 mg, 80 μmol) and Cs₂CO₃ (322 mg, 0.99 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (EtOAc/pet. ether) to give imidazopyridinone 195 (110 mg, 63%) as a tan powder: mp (EtOAc/pet. ether) 139-141° C.; ¹H NMR (CDCl₃) δ 7.82 (d, J=0.6 Hz, 1H, H-4), 7.29 (ddd, J=9.0, 3.4, 2.2 Hz, 2 H, H-2′, H-6′), 7.17 (d, J=8.6 Hz, 1H, H-6″), 6.97 (ddd, J=9.0, 3.4, 2.2 Hz, 2 H, H-3′, H-5′), 6.77 (d, J=2.9 Hz, 1H, H-3″), 6.69 (dd, J=8.6, 2.9 Hz, 1 H, H-5″), 6.06 (d, J=0.6 Hz, 1H, H-7), 5.97 (br s, 1H, 6-NH), 3.83 (s, 3H, 4′-OCH₃), 3.78 (s, 3H, 4″-OCH₃), 3.45 (s, 3H, 3-CH₃), 2.21 (s, 3H, 2″-CH₃); MS m/z 391.2 (MH⁺, 100%); HRMS calcd for C₂₂H₂₃N₄O₃ (MH⁺) m/z 391.1765, found 391.1757 (2.0 ppm). HPLC purity 99.8%.

### Example 128: SN39529 6-((4-Chloro-2-methylphenyl)amino)-1-(4-methoxyphenyl)-3-methyl-1,3-dihydro-2H-imidazo[4,5-c]pyridin-2-one (196)

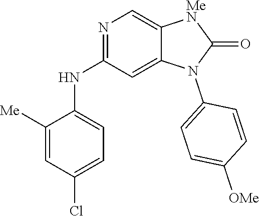 [SMILES: COc1ccc(-n2c(=O)n(C)c3cnc(Nc4ccc(Cl)cc4C)cc32)cc1]

A degassed mixture of chloride 194 (164 mg, 0.57 mmol), 4-chloro-2-methylaniline (89 mg, 0.62 mmol), Pd₂dba₃ (24 mg, 26 μmol), XPhos (50 mg, 104 μmol) and Cs₂CO₃ (373 mg, 1.14 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (50-60% EtOAc/pet. ether) to give imidazopyridinone 196 (153 mg, 74%) as a white powder; ¹H NMR (CDCl₃) δ 7.87 (d, J=0.5 Hz, 1H, H-4), 7.35 (d, J=8.5 Hz, 1H, H-6″), 7.32 (ddd, J=9.0, 3.4, 2.2 Hz, 2 H, H-2′, H-6′), 7.17 (d, J=2.5 Hz, 1H, H-3″), 7.10 (dd, J=8.5, 2.5 Hz, 1 H, H-5″), 6.99 (ddd, J=9.0, 3.4, 2.2 Hz, 2 H, H-3′, H-5′), 6.32 (d, J=0.6 Hz, 1H, H-7), 6.02 (br s, 1H, 6-NH), 3.85 (s, 3H, 4′-OCH₃), 3.47 (s, 3H, 3-CH₃), 2.22 (s, 3H, 2″-CH₃); MS m/z 395.1 (MH⁺, 100%). HRMS calcd for C₂₁H₂O³⁵ClN₄O₂ (MH⁺) m/z 395.1269, found 395.1276 (−1.7 ppm); calcd for C₂₁H₂₀³⁷ClN₄O₂ (MH⁺) m/z 397.1248, found 397.1251 (−0.7 ppm). HPLC purity 98.3%.

### Example 129: SN39522 1-(4-Methoxyphenyl)-3-methyl-6-((2-methyl-5-(methylsulfonyl)phenyl)amino)-1,3-dihydro-2H-imidazo[4,5-c]pyridin-2-one (197)

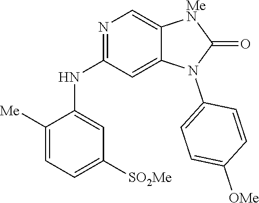 [SMILES: COc1ccc(-n2c(=O)n(C)c3cnc(Nc4cc(S(C)(=O)=O)ccc4C)cc32)cc1]

A degassed mixture of chloride 194 (124 mg, 0.43 mmol), 2-methyl-5-(methylsulfonyl)aniline (95 mg, 0.51 mmol), Pd₂dba₃ (20 mg, 22 μmol), XPhos (41 mg, 86 μmol) and Cs₂CO₃ (308 mg, 0.95 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (EtOAc/pet. ether) to give imidazopyridinone 197 (34 mg, 18%) as a tan powder: mp (EtOAc/pet. ether) 252-255° C.; ¹H NMR (CDCl₃) δ 8.20 (d, J=1.8 Hz, 1H, H-6′), 7.94 (s, 1H, H-4), 7.44 (dd, J=7.9, 1.8 Hz, 1H, H-4′), 7.40 (ddd, J=9.0, 3.4, 2.2 Hz, 2 H, H-2″, H-6″), 7.34 (d, J=7.9 Hz, 1 H, H-3′), 7.04 (ddd, J=9.0, 3.4, 2.2 Hz, 2 H, H-3″, H-5″), 6.58 (s, 1H, H-7), 6.22 (br s, 1H, 6-NH), 3.84 (s, 3H, 4″-OCH₃), 3.50 (s, 3H, 3-CH₃), 3.00 (s, 3H, 5′-SO₂CH₃), 2.33 (s, 3H, 2′-CH₃); MS m/z 439.2 (MH⁺, 100%); HRMS calcd for C₂₂H₂₃N₄O₄S (MH⁺) m/z 439.1435, found 439.1429 (1.2 ppm). HPLC purity 97.4%.

### Example 130: SN39520 1-(4-Methoxyphenyl)-3-methyl-6-((2-methyl-4-(methylsulfonyl)phenyl)amino)-1,3-dihydro-2H-imidazo[4,5-c]pyridin-2-one (198)

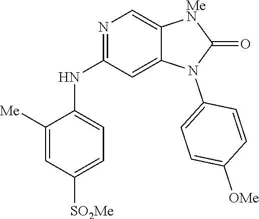 [SMILES: COc1ccc(-n2c(=O)n(C)c3cnc(Nc4ccc(S(C)(=O)=O)cc4C)cc32)cc1]

A degassed mixture of chloride 194 (158 mg, 0.55 mmol), 2-methyl-4-(methylsulfonyl)aniline (121 mg, 0.65 mmol), Pd₂dba₃ (25 mg, 28 μmol), XPhos (52 mg, 110 μmol) and Cs₂CO₃ (394 mg, 1.21 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (EtOAc/pet. ether) to give imidazopyridinone 198 (216 mg, 90%) as a white powder: mp (EtOAc/pet. ether) 236-239° C.; ¹H NMR (CDCl₃) δ 7.98 (s, 1H, H-4), 7.82 (d, J=8.5 Hz, 1H, H-6′), 7.36 (ddd, J=9.0, 3.4, 2.2 Hz, 2 H, H-2″, H-6″), 7.04 (ddd, J=9.0, 3.4, 2.2 Hz, 2 H, H-3″, H-5″), 7.66-7.72 (m, 2H, H-3′, H-5′), 6.60 (s, 1H, H-7), 6.36 (br s, 1H, 6-NH), 3.86 (s, 3H, 4″-OCH₃), 3.52 (s, 3H, 3-CH₃), 3.02 (s, 3H, 4′-SO₂CH₃), 2.32 (s, 3H, 2′-CH₃); MS m/z 439.2 (MH⁺, 100%); HRMS calcd for C₂₂H₂₃N₄O₄S (MH⁺) m/z 439.1435, found 439.1428 (1.5 ppm). HPLC purity 99.0%.

### Example 131: SN39526 6-((4-(Benzyloxy)-2-methylphenyl)amino)-1-(4-methoxyphenyl)-3-methyl-1,3-dihydro-2H-imidazo[4,5-c]pyridin-2-one (199)

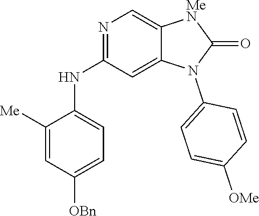 [SMILES: COc1ccc(-n2c(=O)n(C)c3cnc(Nc4ccc(OCc5ccccc5)cc4C)cc32)cc1]

(*). A degassed mixture of chloride 194 (164 mg, 0.57 mmol), 4-(benzyloxy)-2-methylaniline (145 mg, 0.68 mmol), Pd₂dba₃ (26 mg, 29 μmol), XPhos (54 mg, 114 μmol) and Cs₂CO₃ (409 mg, 1.25 mmol) in dioxane (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 ml) and water (50 mL). The organic fraction was washed with water (30 mL), washed with brine (30 mL), dried (MgSO₄), filtered and the solvent evaporated. The residue was purified by chromatography (60-100% EtOAc/pet. ether) to give (i) starting material 194 (65 mg, 40%); and imidazopyridinone 199 (ii) (106 mg, 40%) as a purple foam: ¹H NMR (CDCl₃) δ 7.82 (s, 1H, H-4), 7.36-7.44 (m, 4H, H-2″′, H-3″′, H-5″′, H-6″′), 7.32-7.35 (m, 1H, H-4″′), 7.29 (ddd, J=9.0, 3.3, 2.2 Hz, 2 H, H-2′, H-6′), 7.18 (d, J=8.7 Hz, 1H, H-6″), 6.97 (ddd, J=9.0, 3.3, 2.2 Hz, 2 H, H-3′, H-5′), 6.86 (d, J=2.9 Hz, 1H, H-3″), 6.77 (dd, J=8.7, 2.9 Hz, 1 H, H-5″), 6.08 (d, J=0.6 Hz, 1H, H-7), 5.96 (br s, 1H, 6-NH), 5.03 (s, 2H, CH₂O), 3.84 (s, 3H, 4′-OCH₃), 3.45 (s, 3H, 3-CH₃), 2.21 (s, 3H, 2″-CH₃); MS m/z 467.2 (MH⁺, 100%); HRMS calcd for C₂₈H₂₇N₄O₃ (MH⁺) m/z 467.2028, found 467.2071 (1.4 ppm). HPLC purity 98.4%.

### Example 132: SN39530 6-((4-Hydroxy-2-methylphenyl)amino)-1-(4-methoxyphenyl)-3-methyl-1,3-dihydro-2H-imidazo[4,5-c]pyridin-2-one (200)

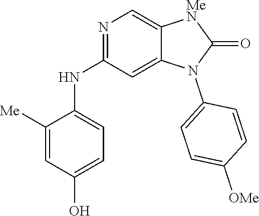 [SMILES: COc1ccc(-n2c(=O)n(C)c3cnc(Nc4ccc(O)cc4C)cc32)cc1]

A mixture of benzyl ether 199 (90 mg) and Pd/C (20 mg) in EtOH/EtOAc (2:1, 75 mL) was stirred under H₂ (50 psi) for 16 h. The mixture was filtered through diatomaceous earth and the pad washed with EtOH (25 mL). The solvent was evaporated and the residue purified by chromatography, eluting with a gradient of (50-100% EtOAc/pet. ether), to give imidazopyridinone 200 (40 mg, 56%) as a brown foam; ¹H NMR (CDCl₃) δ 7.81 (d, J=0.4 Hz, 1 H, H-4), 7.28 (ddd, J=9.0, 3.4, 2.2 Hz, 2 H, H-2′, H-6′), 7.06 (d, J=8.5 Hz, 1H, H-6″), 6.95 (ddd, J=9.0, 3.4, 2.2 Hz, 2 H, H-3′, H-5′), 6.68 (d, J=2.8 Hz, 1H, H-3″), 6.61 (dd, J=8.5, 2.8 Hz, 1 H, H-5″), 6.03 (d, J=0.6 Hz, 1H, H-7), 5.99 (br s, 1H, 6-NH), 3.81 (s, 3H, 4′-OCH₃), 3.45 (s, 3H, 3-CH₃), 2.13 (s, 3H, 2″-CH₃); MS m/z 377.2 (MH⁺, 100%). HRMS calcd for C₂₁H₂₁N₄O₃ (MH⁺) m/z 377.1608, found 377.1611 (−0.8 ppm). HPLC purity 96.4%.

### Example 133: SN39525 1-(4-(Benzyloxy)phenyl)-6-((4-methoxy-2-methylphenyl)amino)-3-methyl-1,3-dihydro-2H-imidazo[4,5-c]pyridin-2-one (205)

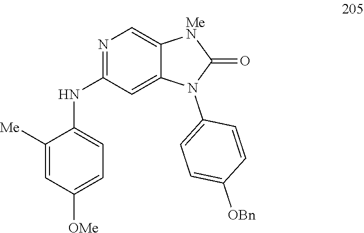 [SMILES: COc1ccc(Nc2cc3c(cn2)n(C)c(=O)n3-c2ccc(OCc3ccccc3)cc2)c(C)c1]

N-(4-(Benzyloxy)phenyl)-2-chloro-5-nitropyridin-4-amine (201). A solution of 4-(benzyloxy)aniline (1.32 g, 5.61 mmol) in dry DCM (10 mL) was added to a stirred solution of nitropyridine 2 (1.03 g, 5.34 mmol) and iPr₂NEt (2.09 mL, 12.0 mmol) in dry DCM (50 mL) at 20° C. The mixture was stirred at 20° C. for 16 h before being diluted with DCM (100 mL) and washed with water (3×100 mL), dried (MgSO₄) and the solvent evaporated. The residue was purified by chromatography, eluting with a gradient (10-100%) of EtOAc/pet. ether, to give chloride 201 (1.86 g, 97%) as yellow crystals: mp 189-191° C.; ¹H NMR (CDCl₃) δ 9.52 (br s, 1H, 4-NH), 9.08 (s, 1H, H-6), 7.44-7.47 (m, 2H, H-3″, H-5″), 7.41 (br dd, J=7.7, 1.7 Hz, 2 H, H-2″, H-6″), 7.37 (br t, J=7.0, 1.7 Hz, 1H, H-4″), 7.19 (ddd, J=8.8, 3.3, 2.1 Hz, 2 H, H-2′, H-6′), 7.08 (ddd, J=8.8, 3.3, 2.2 Hz, 2 H, H-3′, H-5′), 6.78 (s, 1H, H-3) 5.12 (s, 2H, CH₂O); MS m/z 256.1 (MH⁺, 100%), 258.1 (MH⁺, 35%).

N⁴-(4-(Benzyloxy)phenyl)-6-chloropyridine-3,4-diamine (202). A solution of nitropyridine 201 (1.75 g, 4.93 mmol) in EtOAc (50 ml) was added drop wise to a stirred suspension of SnCl₂·2H₂O (4.45 g, 19.7 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 202 (1.61 g, 100%) as a grey foam: ¹H NMR (CDCl₃) δ 7.74 (s, 1H, H-2), 7.41-7.47 (m, 2H, H-3″, H-5″), 7.39 (br dd, J=7.6, 1.6 Hz, 2 H, H-2″, H-6″), 7.34 (br t, J=7.1 Hz, 1H, H-4″), 7.09 (ddd, J=8.9, 3.3, 2.3 Hz, 2 H, H-2′, H-6′), 7.00 (ddd, J=8.9, 3.3, 2.3 Hz, 2 H, H-3′, H-5′), 6.71 (s, 1H, H-5), 5.93 (br s, 1H, 4-NH), 5.08 (s, 2H, CH₂O), 3.20 (br s, 2H, 3-NH₂); MS m/z 326.1 (MH⁺, 100%), 228.1 (MH⁺, 35%).

1-(4-(Benzyloxy)phenyl)-6-chloro-1,3-dihydro-2H-imidazo[4,5-c]pyridin-2-one (203). CDI (0.88 g, 5.42 mmol) was added to a stirred solution of diamine 202 (1.60 g, 4.90 mmol) in dry MeCN (50 mL) at 20° C. The mixture was stirred at 20° C. for 48 h. The solvent was evaporated and the residue partitioned between CHCl₃ (200 mL) and water (100 mL). The organic fraction was washed with water (2×50 mL), washed with brine (50 mL), dried (MgSO₄), filtered and the solvent evaporated. The residue was precipitated from 20% EtOAc/pet. ether, to give the pyridinone 203 (1.73 g, 100%) as a grey powder: mp 301-303° C.; ¹H NMR (CDCl₃) 0 11.57 (br s, 1H, 3-H), 8.05 (s, 1H, H-4), 7.39-7.50 (m, 6H, H-2′, H-6′, H-2″, H-3″, H-5″, H-6″), 7.35 (br t, J=7.2 Hz, 1H, H-4″), 7.18 (ddd, J=9.0, 3.2, 2.0 Hz, 2 H, H-3′, H-5′), 6.92 (s, 1H, H-7), 5.18 (s, 2H, CH₂O); MS m/z 352.1 (MH⁺, 100%), 354.1 (MH⁺, 35%).

1-(4-(Benzyloxy)phenyl)-6-chloro-3-methyl-1,3-dihydro-2H-imidazo[4,5-c]pyridin-2-one (204). NaH (60% dispersion, 131 mg, 3.27 mmol) was added to a stirred solution of pyridinone 203 (0.96 g, 2.73 mmol) and MeI (0.26 mL, 4.10 mmol) in dry DMF (20 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was triturated with pet. ether and dried to give chloride 204 (0.90 g, 90%) as white powder: mp 201-203° C.; ¹H NMR (CDCl₃) δ 8.04 (s, 1H, H-4), 7.39-7.47 (m, 4H, H-2″, H-3″, H-5″, H-6″), 7.33-7.38 (m, 3H, H-2′, H-6′, H-4″), 7.13 (ddd, J=9.0, 3.3, 2.2 Hz, 2 H, H-3′, H-5′), 6.95 (s, 1H, H-7), 5.13 (s, 2H, CH₂O), 3.52 (s, 3H, 3-CH₃); MS m/z 366.1 (MH⁺, 100%), 368.1 (MH⁺, 35%).

1-(4-(Benzyloxy)phenyl)-6-((4-methoxy-2-methylphenyl)amino)-3-methyl-1,3-dihydro-2H-imidazo[4,5-c]pyridin-2-one (205). A degassed mixture of chloride 204 (149 mg, 0.41 mmol), 4-methoxy-2-methylaniline (67 mg, 0.49 mmol), Pd₂dba₃ (19 mg, 21 μmol), XPhos (39 mg, 82 μmol) and Cs₂CO₃ (294 mg, 0.90 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with EtOAc, to give (i) starting material 204 (69 mg, 46%); and (ii) imidazopyridinone 205 (52 mg, 27%) as a tan foam: ¹H NMR (CDCl₃) δ 7.82 (d, J=0.4 Hz, 1H, H-4), 7.38-7.46 (m, 4H, H-2″′, H-3″′, H-5″′, H-6″′), 7.32-7.36 (m, 1H, H-4″′), 7.29 (ddd, J=9.0, 3.3, 2.2 Hz, 2 H, H-2″, H-6″), 7.17 (d, J=8.6 Hz, 1H, H-6′), 7.05 (ddd, J=9.0, 3.4, 2.2 Hz, 2 H, H-3″, H-5″), 6.77 (d, J=2.9 Hz, 1H, H-3′), 6.70 (dd, J=8.6, 2.9 Hz, 1 H, H-5′), 6.07 (d, J=0.5 Hz, 1H, H-7), 5.96 (br s, 1H, 6-NH), 5.08 (s, 2H, CH₂O), 3.78 (s, 3H, 4′-OCH₃), 3.45 (s, 3H, 3-CH₃), 2.21 (s, 3H, 2′-CH₃); MS m/z 467.2 (MH⁺, 100%). HRMS calcd for C₂₈H₂₇N₄O₃ (MH⁺) m/z 467.2078, found 467.2087 (−1.9 ppm). HPLC purity 97.9%.

### Example 134: SN39528 1-(4-Hydroxyphenyl)-6-((4-methoxy-2-methylphenyl)amino)-3-methyl-1,3-dihydro-2H-imidazo[4,5-c]pyridin-2-one (206)

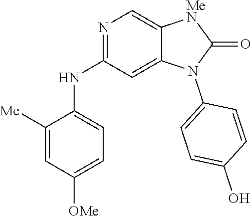 [SMILES: COc1ccc(Nc2cc3c(cn2)n(C)c(=O)n3-c2ccc(O)cc2)c(C)c1]

A mixture of benzyl ether 205 (30 mg) and Pd/C (10 mg) in EtOH/EtOAc (2:1, 60 mL) was stirred under H₂ (50 psi) for 6 h. The mixture was filtered through diatomaceous earth and the pad washed with EtOH (25 mL). The solvent was evaporated and the residue purified by chromatography, eluting with a gradient of (50-100% EtOAc/pet. ether), to give imidazopyridinone 206 (20 mg, 83%) as a brown solid: mp (EtOAc/pet. ether) 246-249° C.; 1H NMR (CDCl₃) δ 7.83 (d, J=0.4 Hz, 1H, H-4), 7.15 (d, J=8.6 Hz, 1H, H-6′), 7.00 (ddd, J=8.8, 3.4, 2.2 Hz, 2 H, H-2″, H-6″), 6.74-6.80 (m, 3H, H-3′, H-3″, H-5″), 6.68 (dd, J=8.6, 2.9 Hz, 1 H, H-5′), 6.05 (br s, 1H, 6-NH), 6.00 (d, J=0.5 Hz, 1H, H-7), 3.77 (s, 3H, 4′-OCH₃), 3.47 (s, 3H, 3-CH₃), 2.17 (s, 3H, 2′-CH₃), 4″-OH not observed; MS m/z 377.2 (MH⁺, 100%). HRMS calcd for C₂₁H₂₁N₄O₃ (MH⁺) m/z 377.1608, found 377.1612 (−1.0 ppm). HPLC purity 98.1%.

### Example 135: SN39547 2-(4-(6-((4-Methoxy-2-methylphenyl)amino)-3-methyl-2-oxo-2,3-dihydro-1H-imidazo[4,5-c]pyridin-1-yl)phenyl)-2-methylpropanenitrile (211)

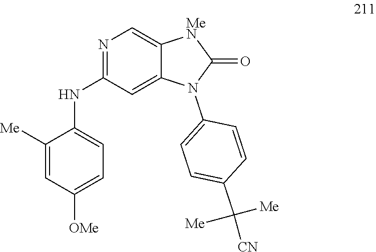 [SMILES: COc1ccc(Nc2cc3c(cn2)n(C)c(=O)n3-c2ccc(C(C)(C)C#N)cc2)c(C)c1]

2-(4-((2-Chloro-5-nitropyridin-4-yl)amino)phenyl)-2-methylpropanenitrile (207). A solution of 2-(4-aminophenyl)-2-methylpropanenitrile (0.92 g, 5.71 mmol) in dry dioxane (10 mL) was added to a stirred solution of nitropyridine 2 (1.05 g, 5.44 mmol) and iPr₂NEt (1.14 mL, 6.53 mmol) in dry dioxane (50 mL) at 20° C. The mixture was stirred at 100° C. for 16 h before being diluted with dioxane (100 mL) and washed with water (3×100 mL), dried (MgSO₄) and the solvent evaporated. The residue was purified by chromatography, eluting with a gradient (10-30%) of EtOAc/pet. ether, to give chloride 207 (1.67 g, 97%) as yellow plates: mp 160-161° C.; ¹H NMR (CDCl₃) δ 9.64 (br s, 1H, 4′-NH), 9.11 (s, 1H, H-6″), 7.62 (br d, J=8.6 Hz, 2 H, H-3′, H-5′), 7.32 (br d, J=8.6 Hz, 2 H, H-2′, H-6′), 6.94 (s, 1H, H-3″), 1.78 (s, 6H, 2×CH₃); MS m/z 317.1 (MH⁺, 100%), 319.1 (MH⁺, 35%).

2-(4-((5-Amino-2-chloropyridin-4-yl)amino)phenyl)-2-methylpropanenitrile (208). A solution of nitropyridine 207 (1.61 g, 5.09 mmol) in EtOAc (50 ml) was added drop wise to a stirred suspension of SnCl₂·2H₂O (4.60 g, 20.37 mmol) in EtOAc (100 mL) at 50° C. while maintaining the temperature below 60° C. The mixture was stirred at 60° C. for 2 h and then cooled to 5° C. and conc. aq. NH₃ solution added until the solution was basic (pH 9). The resulting precipitate was filtered and washed with EtOAc (100 mL). The combined organic fraction was dried (MgSO₄), filtered and the solvent evaporated to give diamine 208 as tan plates: mp 222-225° C.; ¹H NMR (CDCl₃) δ 7.38 (br s, 1H, 4′-NH), 7.64 (s, 1H, H-6″), 7.48 (dd, J=8.7, 2.0 Hz, 2 H, H-3′, H-5′), 7.21 (dd, J=8.7, 2.0 Hz, 2 H, H-2′, H-6′), 6.80 (s, 1H, H-3″), 5.05 (br s, 2H, 5″-NH₂), 1.68 (s, 6H, 2×CH₃); MS m/z 287.1 (MH⁺, 100%), 289.2 (MH⁺, 35%).

2-(4-(6-Chloro-2-oxo-2,3-dihydro-1H-imidazo[4,5-c]pyridin-1-yl)phenyl)-2-methylpropanenitrile (209). CDI (0.78 g, 4.82 mmol) was added to a stirred solution of diamine 208 (1.26 g, 4.38 mmol) in dry MeCN (100 mL) at 20° C. The mixture was stirred at 20° C. for 48 h. The solvent was evaporated and the residue partitioned between CHCl₃ (100 mL) and water (100 mL). The organic fraction was washed with water (2×50 mL), washed with brine (50 mL), dried (MgSO₄), filtered and the solvent evaporated. The residue was precipitated from 20% EtOAc/pet. ether, to give the pyridinone 209 (1.36 g, 100%) as a cream powder: mp 292-295° C.; ¹H NMR [(CD₃)₂SO] δ 8.07 (d, J=0.5 Hz, 1H, H-4″), 7.72 (ddd, J=8.7, 2.5, 2.1 Hz, 2 H, H-3′, H-5′), 7.62 (ddd, J=8.7, 2.5, 2.1 Hz, 2 H, H-2′, H-6′), 7.08 (d, J=0.5 Hz, 1H, H-7), 1.76 (s, 6H, 2×CH₃), 3-H not observed; MS m/z 313.1 (MH⁺, 100%), 315.1 (MH⁺, 35%).

2-(4-(6-Chloro-3-methyl-2-oxo-2,3-dihydro-1H-imidazo[4,5-c]pyridin-1-yl)phenyl)-2-methylpropanenitrile (210). NaH (60% dispersion, 186 mg, 4.64 mmol) was added to a stirred solution of pyridinone 209 (1.32 g, 4.22 mmol) and MeI (0.34 mL, 5.49 mmol) in dry DMF (40 mL) at 5° C. The mixture was stirred at 20° C. for 16 h and then quenched with ice/water (5 mL). The solvent was evaporated and the residue was partitioned between EtOAc (100 mL) and water (50 ml). The organic fraction was washed with water (2×50 mL), washed with brine (50 mL), dried (MgSO₄), filtered and the solvent evaporated. The residue was triturated with pet. ether and dried to give chloride 210 (1.08 g, 78%) as white powder: mp 229-231° C.; ¹H NMR (CDCl₃) δ 8.07 (d, J=0.6 Hz, 1H, H-4″), 7.68 (dd, J=8.8, 2.1 Hz, 2 H, H-3′, H-5′), 7.52 (dd, J=8.8, 2.1 Hz, 2 H, H-2′, H-6′), 7.03 (d, J=0.6 Hz, 1H, H-7), 3.53 (s, 3H, 3-CH₃), 1.79 (s, 6H, 2×CH₃); MS m/z 327.1 (MH⁺, 100%), 329.1 (MH⁺, 35%).

2-(4-(6-((4-Methoxy-2-methylphenyl)amino)-3-methyl-2-oxo-2,3-dihydro-1H-imidazo[4,5-c]pyridin-1-yl)phenyl)-2-methylpropanenitrile (211). A degassed mixture of chloride 210 (108 mg, 0.33 mmol), 4-methoxy-2-methylaniline (54 mg, 0.40 mmol), Pd₂dba₃ (15 mg, 17 μmol), XPhos (31 mg, 66 μmol) and Cs₂CO₃ (237 mg, 0.73 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with a gradient (70-100%) of EtOAc/pet. ether) to give imidazopyridinone 211 (95 mg, 67%) as tan crystals: mp (EtOAc/pet. ether) 210-211° C.; ¹H NMR (CDCl₃) δ 7.82 (d, J=0.6 Hz, 1H, H-4″), 7.55 (ddd, J=8.7, 2.6, 2.1 Hz, 2 H, H-2′, H-6′), 7.42 (ddd, J=8.7, 2.6, 2.1 Hz, 2 H, H-3′, H-5′), 7.18 (d, J=8.6 Hz, 1H, H-6″′), 6.77 (d, J=2.9 Hz, 1H, H-3″′), 6.71 (dd, J=8.6, 2.9 Hz, 1 H, H-5″′), 6.15 (d, J=0.6 Hz, 1H, H-7), 5.96 (br s, 1H, 6″-NH), 3.76 (s, 3H, 4″′—OCH₃), 3.44 (s, 3H, 3″-CH₃), 2.20 (s, 3H, 2″′-CH₃), 1.73 (s, 6H, 2×CH₃); MS m/z 428.2 (MH⁺, 100%); HRMS calcd for C₂₅H₂₆N₅O₂ (MH⁺) m/z 428.2081, found 428.2088 (−1.5 ppm). HPLC purity 99.3%.

### Example 136: SN39548 2-(4-(6-((4-Chloro-2-methylphenyl)amino)-3-methyl-2-oxo-2,3-dihydro-1H-imidazo[4,5-c]pyridin-1-yl)phenyl)-2-methylpropanenitrile (212)

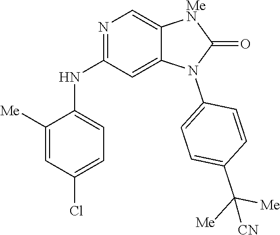 [SMILES: Cc1cc(Cl)ccc1Nc1cc2c(cn1)n(C)c(=O)n2-c1ccc(C(C)(C)C#N)cc1]

A degassed mixture of chloride 210 (120 mg, 0.37 mmol), 4-chloro-2-methylaniline (62 mg, 0.44 mmol), Pd₂dba₃ (17 mg, 19 μmol), XPhos (35 mg, 74 μmol) and Cs₂CO₃ (265 mg, 0.81 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with a gradient (50-100%) of EtOAc/pet. ether, to give imidazopyridinone 212 (92 mg, 58%) as tan needles: mp (EtOAc/pet. ether) 229-231° C.; ¹H NMR (CDCl₃) δ 7.82 (s, 1H, H-4″), 7.60 (ddd, J=8.7, 2.6, 2.1 Hz, 2 H, H-2′, H-6′), 7.46 (ddd, J=8.7, 2.6, 2.1 Hz, 2 H, H-3′, H-5′), 7.36 (d, J=8.6 Hz, 1H, H-6), 7.17 (d, J=2.2 Hz, 1H, H-3″′), 6.71 (dd, J=8.6, 2.4 Hz, 1 H, H-5″′), 6.40 (d, J=0.6 Hz, 1H, H-7), 6.02 (br s, 1H, 6″-NH), 3.47 (s, 3H, 3″-CH₃), 2.20 (s, 3H, 2″′-CH₃), 1.75 (s, 6H, 2×CH₃); MS m/z 432.2 (MH⁺, 100%), 434.2 (MH⁺, 100%); HRMS calcd for C₂₄H₂₃ClN₅O (MH⁺) m/z 432.1586, found 432.1590 (−1.1 ppm). HPLC purity 97.5%.

### Example 137: SN39546 2-Methyl-2-(4-(3-methyl-2-oxo-6-(quinolin-6-ylamino)-2,3-dihydro-1H-imidazo[4,5-c]pyridin-1-yl)phenyl)propanenitrile (213)

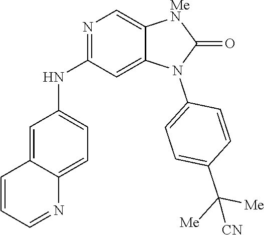 [SMILES: Cn1c(=O)n(-c2ccc(C(C)(C)C#N)cc2)c2cc(Nc3ccc4ncccc4c3)ncc21]

A degassed mixture of chloride 210 (106 mg, 0.32 mmol), quinolin-6-amine (56 mg, 0.39 mmol), Pd₂dba₃ (15 mg, 16 μmol), XPhos (31 mg, 64 μmol) and Cs₂CO₃ (229 mg, 0.70 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 213 (107 mg, 69%) as a cream powder: mp (EtOAc/pet. ether) 280-282° C.; ¹H NMR (CDCl₃) δ 8.71 (dd, J=4.2, 1.6 Hz, 1H, H-2″′), 7.97-8.05 (m, 3H, H-4″, H-4″′, H-7″′), 7.91 (d, J=2.4 Hz, 1H, H-5″′), 7.63 (ddd, J=8.7, 2.4, 2.1 Hz, 2 H, H-2′, H-6′), 7.52-7.56 (m, 3H, H-3′, H-5′, H-3″′), 7.30 (dd, J=8.3, 4.2 Hz, 1H, H-8″′), 6.68 (br s, 1 H, 6″-NH), 6.62 (s, 1H, H-7), 3.49 (s, 3H, 3″-CH₃), 1.72 (s, 6H, 2×CH₃); MS m/z 435.2 (MH⁺, 100%); HRMS calcd for C₂₆H₂₃N₆O (MH⁺) m/z 435.1928, found 435.1929 (−0.2 ppm). HPLC purity 99.9%.

### Example 138: SN39549 2-Methyl-2-(4-(3-methyl-2-oxo-6-(quinoxalin-6-ylamino)-2,3-dihydro-1H-imidazo[4,5-c]pyridin-1-yl)phenyl)propanenitrile (214)

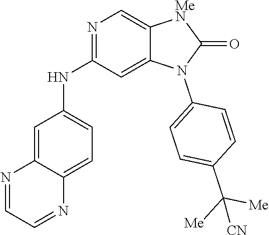 [SMILES: Cn1c(=O)n(-c2ccc(C(C)(C)C#N)cc2)c2cc(Nc3ccc4nccnc4c3)ncc21]

A degassed mixture of chloride 210 (112 mg, 0.34 mmol), quinoxalin-6-amine (60 mg, 0.41 mmol), Pd₂dba₃ (16 mg, 17 μmol), XPhos (32 mg, 68 μmol) and Cs₂CO₃ (244 mg, 0.75 mmol) in MeCN (6 mL) was stirred in a sealed tube at 120° C. for 16 h. The mixture was cooled, diluted with EtOAc (30 mL), filtered through diatomaceous earth and the filtrate was evaporated. The residue was partitioned between EtOAc (50 mL) and water (50 mL). The organic fraction was washed with water (30 mL), washed with brine (30 mL), dried (MgSO₄), filtered and the solvent evaporated. The residue was purified by chromatography, eluting with EtOAc, to give imidazopyridinone 214 (5 mg, 3%) as a yellow powder: ¹H NMR (CDCl₃) δ 8.72 (d, J=1.9 Hz, 1H, H-2″′), 8.64 (d, J=1.9 Hz, 1H, H-3″′), 8.15 (d, J=2.0 Hz, 1H, H-5), 8.03 (s, 1H, H-4″), 7.96 (d, J=9.1 Hz, 1H, H-8″′), 7.60 (m, 3H, H-3′, H-5′, H-7), 7.54 (br ddd, J=8.7, 2.5, 2.0 Hz, 2 H, H-2′, H-6′), 6.85 (br s, 1H, 6″-NH), 6.76 (s, 1H, H-7), 3.53 (s, 3H, 3″-CH₃), 1.75 (s, 6H, 2-CH₃, H-3); MS m/z 436.2 (MH⁺, 100%). HRMS calcd for C₂₅H₂₂N₇O (MH⁺) m/z 436.1897, found 436.1897 (−0.0 ppm). HPLC purity 94.3%.

### Example 139: SN40230 6-((4-Methoxyphenyl)amino)-3-methyl-1-(tetrahydro-2H-pyran-4-yl)-1,3-dihydro-2H-imidazo[4,5-c]pyridin-2-one (215)

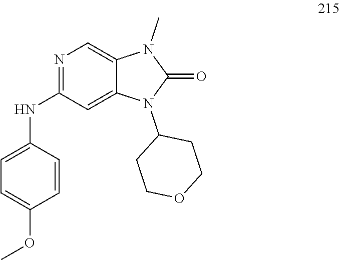 [SMILES: COc1ccc(Nc2cc3c(cn2)n(C)c(=O)n3C2CCOCC2)cc1]

A degassed mixture of chloride 120 (100 mg, 0.37 mmol), 4-methoxyaniline (50 mg, 0.44 mmol), Pd₂dba₃ (20 mg, 19 μmol), XPhos (35 mg, 74 μmol) and Cs₂CO₃ (270 mg, 0.81 mmol) in dioxane (3 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, to give imidazopyridinone 215 (69 mg, 53%) as a tan solid: mp 216-219° C.; ¹H NMR (CDCl₃) δ 7.80 (s, 1H, 4-H), 7.22 (m, 2H, H-3″, H-5″), 6.91 (m, 2H, H-2″, H-6″), 6.52 (d, J=0.6 Hz, 1H, H-7), 6.25 (s, 1H, NH), 4.46 (tt, J=12.4, 4.2 Hz, 1H, H-4′), 4.09 (br dd, J=11.5, 4.4 Hz, 2 H, H-2′a, H-6′a), 3.83 (s, 3H, OCH₃), 3.51 (td, J=12.1, 1.7 Hz, 2 H, H-2′a, H-6′a), 3.39 (s, 3H, NCH₃), 2.39-2.25 (app. qd, J=12.6, 4.7, 2 H, H-3′b, H-5′b), 1.72 (dd, J=12.5, 2.4 Hz, 2 H, H-3′b, H-5′b). HRMS calcd for C₁₉H₂₃N₄O₃ (MH⁺) m/z 355.1765, found 355.1768 (+0.84 ppm). HPLC purity 98.6%

### Example 140: SN40284 6-((4,5-Dimethoxy-2-methylphenyl)amino)-3-methyl-1-(tetrahydro-2H-pyran-4-yl)-1,3-dihydro-2H-imidazo[4,5-c]pyridin-2-one (216)

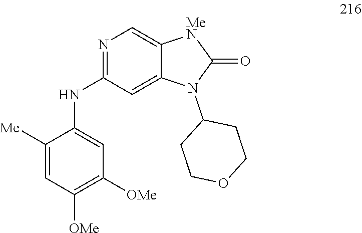 [SMILES: COc1cc(C)c(Nc2cc3c(cn2)n(C)c(=O)n3C2CCOCC2)cc1OC]

4,5-Dimethoxy-2-methylaniline. To 2-amino-4,5-dimethoxybenzoic acid (0.6 g, 3.03 mmol) in dioxane (45 mL) at 0° C. was added LiAlH₄ (0.93 g, 24.3 mmol) and the resulting mixture was heated at reflux for 4 days, then quenched by careful addition of ice-water. The resulting slurry was filtered through diatomaceous earth and evaporated. The residue was purified by chromatography, eluting with 50% EtOAc/pet. ether to give a semi-pure residue which was further purified with chromatography, eluting with a gradient (0-5%) of EtOAc/ⁱPr₂O followed by trituration with iPr₂O to give 4,5-dimethoxy-2-methylaniline (80 mg, 16%) as a red-tinged solid which was used without further characterisation; MS m/z 167.0 (MH⁺, 100%).

6-((4,5-Dimethoxy-2-methylphenyl)amino)-3-methyl-1-(tetrahydro-2H-pyran-4-yl)-1,3-dihydro-2H-imidazo[4,5-c]pyridin-2-one (216). A degassed mixture of chloride 120 (100 mg, 0.37 mmol), 4,5-dimethoxy-2-methylaniline (62 mg, 0.44 mmol), BrettPhos Pd(II) G3 (20 mg, 19 μmol) and Cs₂CO₃ (270 mg, 0.81 mmol) in dioxane (3 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc/iPr₂O to give imidazopyridinone 216 (55 mg, 37%) as a cream solid: mp 180-181° C.; ¹H NMR [(CD₃)₂SO] δ 7.83 (s, 1H, 4-H), 7.66 (s, 1H, NH), 7.29 (s, 1H, H-6″), 6.79 (s, 1H, H-2″), 6.71 (s, 1H, H-7), 4.37 (tt, J=12.3, 4.2 Hz, 1H, H-4′), 3.97 (br dd, J=11.4, 4.2 Hz, 2 H, H-2′a, H-6′a), 3.72 (s, 3H, 5″-OCH₃), 3.67 (s, 3H, 4″-OCH₃), 3.46 (t, J=10.9 Hz, 2 H, H-2′b, H-6′b), 3.28 (s, 3H, NCH₃), 2.23 (app. qd, J=12.5, 4.6, 2 H, H-3′a, H-5′a), 1.65 (dd, J=12.3, 2.8 Hz, 2 H, H-3′b, H-5′b), 2.15 (s, 3H, 2″-CH₃). HRMS calcd for C₂₁H₂₇N₄O₄ (MH⁺) m/z 399.2027, found 399.2049 (+5.48 ppm). HPLC purity 96.0%

### Example 141: SN40249 6-((5-Chloro-3-methylpyridin-2-yl)amino)-3-methyl-1-(tetrahydro-2H-pyran-4-yl)-1,3-dihydro-2H-imidazo[4,5-c]pyridin-2-one (217)

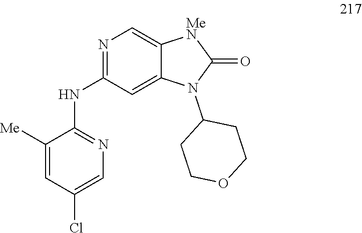 [SMILES: Cc1cc(Cl)cnc1Nc1cc2c(cn1)n(C)c(=O)n2C1CCOCC1]

A degassed mixture of chloride 120 (150 mg, 0.56 mmol), 5-chloro-3-methylpyridin-2-amine (100 mg, 0.67 mmol), BrettPhos Pd(II) G3 (30 mg, 28 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 217 (90 mg, 43%) as a tan solid: mp 238-240° C.; ¹H NMR [(CD₃)₂SO] δ 8.30 (s, 1H, NH), 8.08-8.05 (br m, 1 H, H-6″), 8.04 (s, 1H, H-4), 7.85 (s, 1H, H-7), 7.64-7.61 (br m, 1 H, H-4″), 4.40 (tt, J=12.3, 4.2 Hz, 1H, H-4′), 3.99 (br dd, J=11.4, 4.0 Hz, 2 H, H-2′a, H-6′a), 3.47 (t, J=11.6 Hz, 2 H, H-2′b, H-6′b), 3.34 (s, 3H, NCH₃), 2.35-2.23 (m, 2H, H-3′a, H-5′a), 2.28 (s, 3H 3″-CCH₃), 1.68 (d, J=12.6, 3.0 Hz, 2 H, H-3′b, H-5′b). HRMS calcd for C₁₈H₂₁ClN₅O₂ (MH⁺) m/z 374.1378, found 374.1391 (+3.47 ppm). HPLC purity 98.9%

### Example 142: SN40250 6-((6-Chloro-4-methylpyridin-3-yl)amino)-3-methyl-1-(tetrahydro-2H-pyran-4-yl)-1,3-dihydro-2H-imidazo[4,5-c]pyridin-2-one (218)

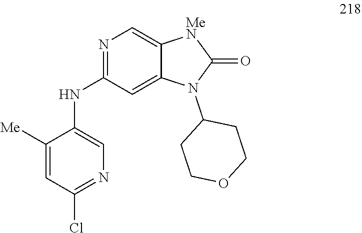 [SMILES: Cc1cc(Cl)ncc1Nc1cc2c(cn1)n(C)c(=O)n2C1CCOCC1]

6-Chloro-4-methylpyridin-3-amine. To 2-chloro-4-methyl-5-nitropyridine (0.6 g, 3.48 mmol) in EtOH (40 mL) was added SnCl₂·2H₂O (1.98 g, 10.44 mmol) and the mixture was heated to reflux for 18 hours, cooled and poured over 1M NaOH. The mixture was extracted with DCM (3×50 mL), dried (Na₂SO₄) and filtered through diatomaceous earth, then solvent evaporated. The residue was purified by chromatography, eluting with a gradient (10%-40%) of EtOAc/pet. ether to give 6-chloro-4-methylpyridin-3-amine (0.28 g, 70%): ¹H NMR (CDCl₃) 7.78 (s, 1H, H-5), 7.00 (s, 1H, H-2), 3.61 (br s, 2H, NH₂), 2.16 (d, J=0.7 Hz, 3 H, 2-CH₃).

6-((6-Chloro-4-methylpyridin-3-yl)amino)-3-methyl-1-(tetrahydro-2H-pyran-4-yl)-1,3-dihydro-2H-imidazo[4,5-c]pyridin-2-one (218). A degassed mixture of chloride 120 (150 mg, 0.56 mmol), 6-chloro-4-methylpyridin-3-amine (100 mg, 0.67 mmol), BrettPhos Pd(II) G3 (30 mg, 28 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 219 (20 mg, 10%) as a tan solid: mp 211-213° C.; ¹H NMR [(CD₃)₂SO] δ 8.97 (s, 1H, H-2″), 8.04 (s, 1H, NH), 7.92 (s, 1H, H-4), 7.31 (s, 1H, H-5″), 7.06 (s, 1H, H-7), 4.42 (tt, J=12.2, 4.1 Hz, 1H, H-4′), 4.01 (br dd, J=11.4, 4.21 Hz, 2 H, H-2′a, H-6′a), 3.49 (t, J=11.3 Hz, 2 H, H-2′b, H-6′b), 3.31 (s, 3H, NCH₃), 2.35-2.22 (m, 2H, H-3′a, H-5′a), 2.28 (s, 3H, 4″-CCH₃), 1.69 (dd, J=12.4, 2.8 Hz, 2 H, H-3′b, H-5′b). HRMS calcd for C₁₈H₂₁ClN₅O₂ (MH⁺) m/z 374.1378, found 374.1389 (+2.94 ppm). HPLC purity 98.7%

### Example 143: SN40251 6-((6-Chloro-2-methylpyridin-3-yl)amino)-3-methyl-1-(tetrahydro-2H-pyran-4-yl)-1,3-dihydro-2H-imidazo[4,5-c]pyridin-2-one (219)

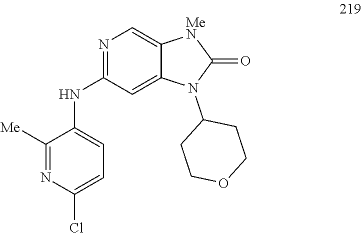 [SMILES: Cc1nc(Cl)ccc1Nc1cc2c(cn1)n(C)c(=O)n2C1CCOCC1]

6-Chloro-2-methylpyridin-3-amine. To 6-chloro-2-methyl-3-nitropyridine (600 mg, 3.48 mmol) in EtOH (40 mL) was added SnCl₂·2H₂O (1.98 g, 10.44 mmol) and the mixture was heated to reflux for 18 hours, cooled and poured over 1M NaOH. The mixture was extracted with DCM (3×50 mL), dried (Na₂SO₄ and filtered through diatomaceous earth, then solvent evaporated. The residue was purified by chromatography, eluting with a gradient (10%-50%) of EtOAc/pet. ether to give 6-chloro-2-methylpyridin-3-amine (280 mg, 50%); ¹H NMR (CDCl₃) 6.98 (d, J=8.2 Hz, 1H, H-4), 6.90 (d, J=8.3 Hz, 1H, H-5), 3.61 (br s, 2H, NH₂), 2.38 (s, 3H, 2-CH₃).

6-((6-Chloro-2-methylpyridin-3-yl)amino)-3-methyl-1-(tetrahydro-2H-pyran-4-yl)-1,3-dihydro-2H-imidazo[4,5-c]pyridin-2-one (219). A degassed mixture of chloride 120 (150 mg, 0.56 mmol), 6-chloro-2-methylpyridin-3-amine (100 mg, 0.67 mmol), Pd₂dba₃ (30 mg, 28 μmol), XPhos (50 mg, 110 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 219 (20 mg, 10%) as a brown solid: mp 230-232° C.; ¹H NMR [(CD₃)₂SO] δ 8.49 (d, J=8.8 Hz, 1H, H-4″), 8.08 (s, 1H, H-4), 7.92 (s, 1H, NH), 7.22 (d, J=8.6 Hz, 1H, H-5″), 7.12 (s, 1H, H-7), 4.42 (tt, J=12.2, 4.2 Hz, 1H, H-4′), 4.01 (br dd, J=11.5, 4.1 Hz, 2 H, H-2′a, H-6′a), 3.49 (t, J=11.8 Hz, 2 H, H-2′b, H-6′b), 3.31 (s, 3H, NCH₃), 2.45 (s, 3H, 3″-CCH₃), 2.29 (ddd, J=24.8, 12.6, 4.6 Hz, 2 H, H-3′a, H-5′a), 1.69 (dd, J=12.3, 3.0 Hz, 2 H, H-3′b, H-5′b). HRMS calcd for C₁₈H₂₁ClN₅O₂ (MH⁺) m/z 374.1378, found 374.1393 (+4.01 ppm). HPLC purity 99.5%

### Example 144: SN40253 6-((5-Methoxy-3-methylpyridin-2-yl)amino)-3-methyl-1-(tetrahydro-2H-pyran-4-yl)-1,3-dihydro-2H-imidazo[4,5-c]pyridin-2-one (220)

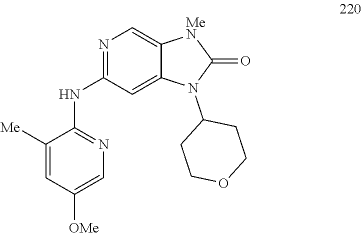 [SMILES: COc1cnc(Nc2cc3c(cn2)n(C)c(=O)n3C2CCOCC2)c(C)c1]

5-Methoxy-3-methylpyridin-2-amine. Sodium (180 mg, 8.03 mmol) was added to anhydrous methanol (10 mL) in a sealed tube and stirred until consumed. 5-Bromo-3-methylpyridin-2-amine (1.0 g, 5.35 mmol) was added, then copper powder (150 mg, 2.36 mmol) and the mixture was heated to 130° C. for 18 h. The resulting slurry was filtered through diatomaceous earth, washing with ethyl acetate and the filtrate evaporated. The residue was purified by chromatography, eluting with a gradient (10-100%) of EtOAc/pet. ether to give 5-methoxy-3-methylpyridin-2-amine (280 mg, 38%): ¹H NMR (CDCl₃) δ 7.66 (br s, 1H, H-6), 6.92 (d, J=2.0 Hz, 1H, H-4), 4.18 (br s, 2H, NH₂), 3.77 (s, 3H, OCH₃), 2.13 (s, 3H, 3-CH₃).

6-((5-Methoxy-3-methylpyridin-2-yl)amino)-3-methyl-1-(tetrahydro-2H-pyran-4-yl)-1,3-dihydro-2H-imidazo[4,5-c]pyridin-2-one (220). A degassed mixture of chloride 120 (150 mg, 0.56 mmol), 5-methoxy-3-methylpyridin-2-amine (90 mg, 0.67 mmol), BrettPhos Pd(II) G3 (30 mg, 28 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 220 (63 mg, 30%) as a white solid: mp 228-232° C.; ¹H NMR [(CD₃)₂SO] δ 7.98 (s, 1H, NH), 7.94 (s, 1H, H-4), 7.81 (d, J=3.1 Hz, 1H, H-6″), 7.66 (s, 1H, H-7), 7.25 (dd, J=3.0, 0.6 Hz, 1H, H-4″), 4.38 (tt, J=12.2, 3.9 Hz, 1H, H-4′), 3.99 (br dd, J=11.4, 4.3 Hz, 2 H, H-2′a, H-6′a) 3.78 (s, 3H, OCH₃), 3.47 (t, J=11.0 Hz, 2 H, H-2′b, H-6′b), 3.31 (s, 3H, NCH₃), 2.36-2.21 (m, 2H, H-3′a, H-5′a), 2.25 (s, 3H, 3″-CCH₃), 1.68 (dd, J=12.6, 3.0 Hz, 2 H, H-3′b, H-5′b). HRMS calcd for C₁₉H₂₄N₅O₃(MH⁺) m/z 370.1874, found 370.1886 (+3.24 ppm). HPLC purity 99.4%

### Example 145: SN40252 6-((6-Methoxy-2-methylpyridin-3-yl)amino)-3-methyl-1-(tetrahydro-2H-pyran-4-yl)-1,3-dihydro-2H-imidazo[4,5-c]pyridin-2-one (221)

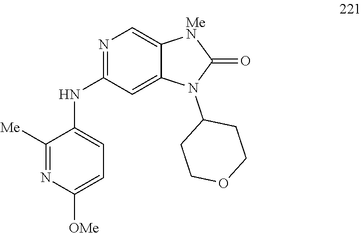 [SMILES: COc1ccc(Nc2cc3c(cn2)n(C)c(=O)n3C2CCOCC2)c(C)n1]

6-Methoxy-2-methyl-3-nitropyridine. Sodium (140 mg, 6.1 mmol) was added to anhydrous methanol (10 mL) and stirred until consumed. 6-Chloro-2-methyl-3-nitropyridine (0.70 g, 4.06 mmol) was added portionwise and the resulting mixture stirred for 18 h at room temperature. The mixture was poured onto ice-water with vigorous stirring, filtered and dried in vacuo to give 6-methoxy-2-methyl-3-nitropyridine (0.52 g, 76%) as a white solid; ¹H NMR (CDCl₃) 8.27 (d, J=9.0 Hz, 1H, H-4), 6.67 (d, J=9.0 Hz, 1H, H-5), 4.02 (s, 3H, OCH₃), 2.82 (s, 3H, 2-CH₃).

6-Methoxy-2-methylpyridin-3-amine. To 6-methoxy-2-methyl-3-nitropyridine (500 mg, 2.97 mmol) in EtOH (40 mL) added SnCl₂·2H₂O (1.69 g, 8.91 mmol) and heated to reflux for 18 hours, cooled and poured over 1M NaOH. The mixture was extracted with DCM (3×50 mL), dried (Na₂SO₄) and filtered through diatomaceous earth, then solvent evaporated. The residue was purified by chromatography, eluting with a gradient (50-100%) of EtOAc/pet. ether to give 6-methoxy-2-methylpyridin-3-amine (260 mg, 63%) as a red oil: ¹H NMR (CDCl₃) 6.95 (d, J=8.5 Hz, 1H, H-4), 6.46 (dd, J=8.4, 0.5 Hz, 1H, 5-H), 3.86 (s, 3H, OCH₃), 3.26 (s, 2H, NH₂), 2.34 (s, 3H, 2-CH₃).

6-((6-Methoxy-2-methylpyridin-3-yl)amino)-3-methyl-1-(tetrahydro-2H-pyran-4-yl)-1,3-dihydro-2H-imidazo[4,5-c]pyridin-2-one (221). A degassed mixture of chloride 120 (150 mg, 0.56 mmol), 6-methoxy-2-methylpyridin-3-amine (90 mg, 0.67 mmol), BrettPhos Pd(II) G3 (30 mg, 28 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 221 (70 mg, 30%) as a white solid: mp 160-162° C.; ¹H NMR [(CD₃)₂SO] δ 7.88 (d, J=8.7 Hz, 1H, H-4″), 7.82 (s, 1H, NH), 7.78 (s, 1H, H-4), 6.74 (s, 1H, 7-H), 6.60 (d, J=8.7 Hz, 1H, H-5″), 4.38 (tt, J=12.2, 4.2 Hz, 1H, H-4′), 3.99 (br dd, J=11.3, 4.2 Hz, 2 H, H-2′a, H-6′a) 3.81 (s, 3H, OCH₃), 3.47 (t, J=11.0 Hz, 2 H, H-2′b, H-6′b), 3.27 (s, 3H, NCH₃), 2.34 (s, 3H, 2″-CCH₃), 2.33-2.20 (m, 2H, H-3′a, H-5′a), 1.67 (dd, J=12.3, 2.7 Hz, 2 H, H-3′b, H-5′b). HRMS calcd for C₁₉H₂₄N₅O₃(MH⁺) m/z 370.1874, found 370.1886 (+3.24 ppm). HPLC purity 91.4%

### Example 146: SN40262 6-((2-Methoxy-4-methylpyrimidin-5-yl)amino)-3-methyl-1-(tetrahydro-2H-pyran-4-yl)-1,3-dihydro-2H-imidazo[4,5-c]pyridin-2-one (222)

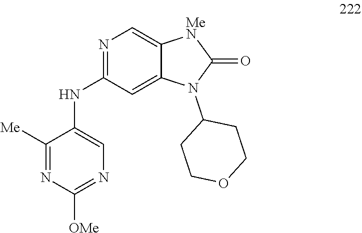 [SMILES: COc1ncc(Nc2cc3c(cn2)n(C)c(=O)n3C2CCOCC2)c(C)n1]

2-Chloro-4-methylpyrimidin-5-amine. To 2,4-dichloro-6-methyl-5-nitropyrimidine (3.0 g, 14.4 mmol) and ammonium chloride (9.43 g, 144.2 mmol) in methanol (30 mL) was added Zn dust (7.71 g, 144.2 mmol) and the resulting slurry stirred at 70° C. for 48 hours, cooled, filtered through diatomaceous earth and solvent evaporated. The residue was purified by chromatography, eluting with a gradient (50-100%) of EtOAc/pet. ether, then the resulting solid was recrystallized from EtOAc to give 2-chloro-4-methylpyrimidin-5-amine (0.80 g, 39%) as a white solid: ¹H NMR (CDCl₃) δ 8.00 (s, 1H, H-6), 3.67 (br s, 2H, NH₂), 2.41 (s, 3H, 4-CH₃).

2-Methoxy-4-methylpyrimidin-5-amine. Sodium (240 g, 10.4 mmol) was added to anhydrous methanol (20 mL) in a sealed tube and stirred until consumed. 2-Chloro-4-methylpyrimidin-5-amine (300 mg, 2.08 mmol) was added and the mixture stirred at 100° C. for 18 h, cooled and evaporated. The residue was purified by chromatography, eluting with a gradient (0-5%) DCM/MeOH to give 2-methoxy-4-methylpyrimidin-5-amine (130 mg, 46%) as a yellow solid: ¹H NMR (CDCl₃) δ 7.84 (s, 1H, H-6), 4.73 (br s, 2H, NH₂), 3.75 (s, 3H, OCH₃), 2.23 (s, 3H, 4-CH₃).

6-((2-Methoxy-4-methylpyrimidin-5-yl)amino)-3-methyl-1-(tetrahydro-2H-pyran-4-yl)-1,3-dihydro-2H-imidazo[4,5-c]pyridin-2-one (222). A degassed mixture of chloride 120 (150 mg, 0.56 mmol), 2-methoxy-4-methylpyrimidin-5-amine (90 mg, 0.67 mmol), BrettPhos Pd(II) G3 (30 mg, 28 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 222 (110 mg, 52%) as a cream solid: mp 212-215° C.; ¹H NMR [(CD₃)₂SO] δ 8.75 (s, 1H, H-6″), 8.00 (s, 1H, NH), 7.81 (s, 1H, H-4), 6.83 (s, 1H, H-7), 4.40 (tt, J=12.3, 4.2 Hz, 1H, H-4′), 4.00 (br dd, J=11.3, 4.1 Hz, 2 H, H-2′a, H-6′a) 3.87 (s, 3H, OCH₃), 3.48 (t, J=11.7 Hz, 2 H, H-2′b, H-6′b), 3.28 (s, 3H, NCH₃), 2.36 (s, 3H, 4″-CCH₃), 2.28 (ddd, J=24.2, 11.6, 4.5 Hz, 2 H, H-3′a, H-5′a), 1.68 (dd, J=13.2, 2.8 Hz, 2 H, H-3′b, H-5′b). HRMS calcd for C₁₈H₂₃N₆O₃ (MH⁺) m/z 371.1826, found 371.1841 (+4.04 ppm). HPLC purity 100%

### Example 147: SN40266 6-((4-(6-Methoxypyridin-3-yl)phenyl)amino)-3-methyl-1-(tetrahydro-2H-pyran-4-yl)-1,3-dihydro-2H-imidazo[4,5-c]pyridin-2-one (223)

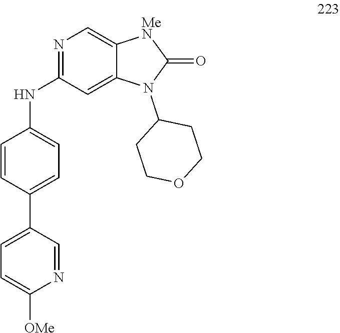 [SMILES: COc1ccc(-c2ccc(Nc3cc4c(cn3)n(C)c(=O)n4C3CCOCC3)cc2)cn1]

4-(6-Methoxypyridin-3-yl)aniline. 4-Bromoaniline (510 mg, 2.94 mmol) and (6-methoxypyridin-3-yl)boronic acid (500 mg, 3.27 mmol) were dissolved in dioxane (20 mL) and the mixture was thoroughly degassed. Bis(triphenylphosphine)palladium chloride (110 mg, 0.16 mmol) and a degassed solution of Na₂CO₃ (1M, 7.8 mL) were added and the mixture heated to reflux for 18 h. Solvent was evaporated and the residue taken up in EtOAc, the aqueous layer extracted with EtOAc (2×50 mL) and the organic layers were washed with brine, dried (Na₂SO₄) and evaporated. The residue was purified by chromatography, eluting with a gradient (5-40%) of EtOAc/pet. ether, followed by recrystallisation from Et₂O/pet. ether to give 4-(6-methoxypyridin-3-yl)aniline (140 mg, 22%) as a brown solid: ¹H NMR [(CD₃)₂SO]δ 8.32 (dd, J=2.6, 0.6 Hz, 1H, H-2′), 7.85 (dd, J=8.6, 2.6 Hz, 1H, H-4′), 7.31 (d, J=8.5 Hz, 2 H, H-3, H-5), 6.81 (dd, J=8.6, 0.6 Hz, 1H, H-5′), 6.63 (d, J=8.6 Hz, 2 H, H-2, H-6), 5.21 (br s, 2H, NH₂), 3.85 (s, 3H, OCH₃).

6-((4-(6-Methoxypyridin-3-yl)phenyl)amino)-3-methyl-1-(tetrahydro-2H-pyran-4-yl)-1,3-dihydro-2H-imidazo[4,5-c]pyridin-2-one (223). A degassed mixture of chloride 120 (150 mg, 0.56 mmol), 4-(6-methoxypyridin-3-yl)aniline (130 mg, 0.67 mmol), BrettPhos Pd(II) G3 (30 mg, 28 μmol) and Cs₂CO₃ (400 mg, 1.2 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 223 (70 mg, 29%) as a tan solid: mp 254-257° C.; ¹H NMR [(CD₃)₂SO] δ 8.95 (s, 1H, NH), 8.43 (dd, J=2.5, 0.5 Hz, 1H, H-2″′), 7.97 (s, 1H, H-4), 7.95 (dd, J=8.7, 2.5 Hz, 1H, H-4″′), 7.74 (d, J=8.8 Hz, 2 H, H-2″, H-6″), 7.54 (d, J=8.8 Hz, 2 H, H-3″, H-5″), 6.87 (dd, J=8.6, 0.5 Hz, 1H, H-5″′), 6.84 (s, 1H, H-7), 4.42 (tt, J=12.2, 4.3 Hz, 1H, H-4′), 4.01 (br dd, J=12.3, 3.3 Hz, 2 H, H-2′a, H-6′a) 3.88 (s, 3H, OCH₃), 3.49 (t, J=11.7 Hz, 2 H, H-2′b, H-6′b), 3.32 (s, 3H, NCH₃), 2.28 (ddd, J=25.0, 12.7, 4.4 Hz, 2 H, H-3′a, H-5′a), 1.70 (dd, J=12.3, 2.7 Hz, 2 H, H-3′b, H-5′b). HRMS calcd for C₂₄H₂₆N₅O₃ (MH⁺) m/z 432.2030, found 432.2048 (+4.06 ppm). HPLC purity 98.7%

### Example 148: SN40291 6-((4-(2-Methoxypyrimidin-5-yl)phenyl)amino)-3-methyl-1-(tetrahydro-2H-pyran-4-yl)-1,3-dihydro-2H-imidazo[4,5-c]pyridin-2-one (224)

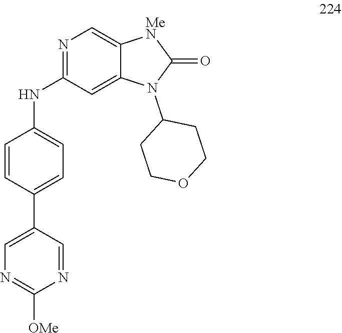 [SMILES: COc1ncc(-c2ccc(Nc3cc4c(cn3)n(C)c(=O)n4C3CCOCC3)cc2)cn1]

4-(2-Methoxypyrimidin-5-yl)aniline. 4-Bromoaniline (560 mg, 3.25 mmol) and (2-methoxypyrimidin-5-yl)boronic acid (500 mg, 3.25 mmol) were dissolved in dioxane (20 mL) and the mixture was thoroughly degassed. Bis(triphenylphosphine)palladium chloride (110 mg, 0.16 mmol) and a degassed solution of Na₂CO₃ (1M, 7.8 mL) were added and the mixture heated to 120° C. for 18 h. The resulting slurry was filtered through diatomaceous earth and the filtrate evaporated. The residue was purified by chromatography, eluting with a gradient (0-20%) of EtOAc/DCM, to give 4-(2-methoxypyrimidin-5-yl)aniline (100 mg, 15%) as a brown foam: ¹H NMR (CDCl₃) δ 8.64 (s, 2H, H-6′, H-4′), 7.32 (d, J=8.5 Hz, H-3, H-5), 6.78 (d, J=8.6 Hz, H-2, H-6), 4.04 (s, 3H, OCH₃), 3.01 (br s, 2H, NH₂).

6-((4-(2-Methoxypyrimidin-5-yl)phenyl)amino)-3-methyl-1-(tetrahydro-2H-pyran-4-yl)-1,3-dihydro-2H-imidazo[4,5-c]pyridin-2-one (224). A degassed mixture of chloride 120 (100 mg, 0.37 mmol), 4-(2-methoxypyrimidin-5-yl)aniline (90 mg, 0.44 mmol), BrettPhos Pd(II) G3 (20 mg, 19 μmol) and Cs₂CO₃ (270 mg, 1.2 mmol) in dioxane (5 mL) was stirred in a sealed tube at 120° C. for 16 h. The mixture was cooled and the mixture diluted with EtOAc (30 mL) and filtered through diatomaceous earth and the filtrate was evaporated. The residue was purified by chromatography, eluting with a gradient (50-100%) of EtOAc/pet. ether, followed by trituration with EtOAc to give imidazopyridinone 224 (20 mg, 13%) as a tan solid: ¹H NMR [(CD₃)₂SO] δ 9.05 (s, 1H, NH), 8.89 (s, 2H, H-4″′, H-6″′), 7.98 (s, 1H, H-4), 7.78 (d, J=8.8 Hz, 2 H, H-2″, H-6″), 7.61 (d, J=8.8 Hz, 2 H, H-3″, H-5″), 6.85 (s, 1H, H-7), 4.42 (tt, J=12.3, 4.2 Hz, 1H, H-4′), 4.01 (br dd, J=11.4, 4.1 Hz, 2 H, H-2′a, H-6′a) 3.95 (s, 3H, OCH₃), 3.49 (t, J=10.6 Hz, 2 H, H-2′b, H-6′b), 3.30 (s, 3H, NCH₃), 2.28 (ddd, J=25.0, 12.6, 4.6 Hz, 2 H, H-3′a, H-5′a), 1.70 (dd, J=12.4, 2.7 Hz, 2 H, H-3′b, H-5′b). HPLC purity 95.6%

### Example 149: SN40318 3-Methyl-6-((7-methylquinoxalin-6-yl)amino)-1-(tetrahydro-2H-pyran-4-yl)-1,3-dihydro-2H-imidazo[4,5-c]pyridin-2-one (225)

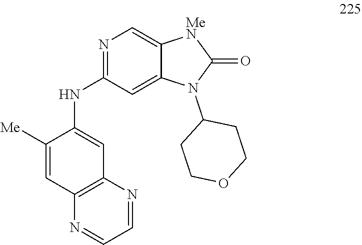 [SMILES: Cc1cc2nccnc2cc1Nc1cc2c(cn1)n(C)c(=O)n2C1CCOCC1]

5-Methyl-2,4-dinitroaniline. To conc. sulfuric acid (18 mL) at 0° C. was added conc. nitric acid (3 mL) followed by N-(m-tolyl)acetamide (5.0 g, 33.5 mmol) and the mixture stirred for 6 h at room temperature, then poured over ice (200 g) and stirred a further 18 h at room temperature. The aqueous mixture was extracted with DCM (3×80 mL) then the combined organic fractions were washed with saturated aqueous NaHCO₃ (200 mL), dried over Na₂SO₄ and solvent was removed in vacuo. The residue was taken up in conc. H₂SO₄ (35 mL) and stirred for 4 h at 80° C., cooled and carefully diluted with water (110 mL), and stirred 18 h. The resulting precipitate was collected by filtration, suspended in EtOAc (200 mL), filtered and the mother liquor evaporated. The residue was purified by chromatography, eluting with a gradient (0-25%) of EtOAc/pet. ether, to give 5-methyl-2,4-dinitroaniline (280 mg, 4%): ¹H NMR [(CD₃)₂SO] δ 8.78 (s, 1H, H-3), 8.16 (br s, 2H, NH₂), 6.92 (d, J=0.7 Hz, H-6), 2.52 (d, J=0.6 Hz, 3 H, CH₃).

4-Methyl-5-nitrobenzene-1,2-diamine. To 5-methyl-2,4-dinitroaniline (200 mg, 1.01 mmol) in EtOH (10 mL) under vigorous reflux conditions, was added Na₂S (350 mg, 4.54 mmol) in water (10 mL) and the temperature allowed to rise to 100° C. before cooling slowly to room temperature and stirring a further 2 h. The resulting mixture was diluted with water (20 mL), extracted with EtOAc (3×20 mL) the combined organic fractions were dried over Na₂SO₄ and solvent was removed in vacuo. The resulting residue was purified by chromatography eluting with 50% EtOAc/pet. ether to give 4-methyl-5-nitrobenzene-1,2-diamine (140 mg, 82%): ¹H NMR [(CD₃)₂SO] δ 7.38 (s, 1H, H-6), 6.37 (s, 1H, H-3), 5.90 (br s, 2H, 2-NH₂), 4.88 (br s, 2H, 1-NH₂), 2.38 (s, 3H, CH₃).

6-Methyl-7-nitroquinoxaline. To 4-methyl-5-nitrobenzene-1,2-diamine (0.2 g, 1.2 mmol) in EtOH (10 mL) added a 40% solution of glyoxal (0.21 mL, 1.44 mmol) and the resulting mixture was stirred at reflux for 18 hours, then solvent was removed in vacuo. The residue was purified by chromatography, eluting with a gradient (5-50%) EtOAc/pet. ether and the resulting product recrystallised from EtOH to give 6-methyl-7-nitroquinoxaline (140 mg, 61%); ¹H NMR (CDCl₃) δ 8.95 (d, J=1.8 Hz, 1H, H-2), 8.93 (d, J 1.7 Hz, 1H, H-3), 8.70 (s, 1H, H-8), 8.08 (br s, 1H, H-5), 2.79 (d, J=0.7 Hz, 3 H, CH₃).

7-Methylquinoxalin-6-amine. To 6-methyl-7-nitroquinoxaline (140 mg, 0.74 mmol) in EtOH (5 mL) and water (1 mL) added iron dust (250 mg, 4.44 mmol) and ammonium chloride (40 mg, 0.74 mmol) and the resulting mixture was stirred at reflux for 0.5 h, cooled, filtered through a plug of diatomaceous earth and solvent was removed in vacuo to yield amino 7-methylquinoxalin-6-amine (120 mg, quant.): ¹H NMR (CDCl₃) δ 8.62 (d, J=1.9 Hz, 1H, H-2), 8.54 (d, J=2.0 Hz, 1H, H-3), 7.77 (br s, 1H, H-8), 7.16 (s, 1H, H-5), 4.19 (br s, 2H, NH₂), 2.42 (d, J=0.6 Hz, 3 H, CH₃).

3-Methyl-6-((7-methylquinoxalin-6-yl)amino)-1-(tetrahydro-2H-pyran-4-yl)-1,3-dihydro-2H-imidazo[4,5-c]pyridin-2-one (225). A degassed mixture of chloride 120 (150 mg, 0.56 mmol), 7-methylquinoxalin-6-amine (110 mg, 0.67 mmol), BrettPhos Pd(II) G3 (30 mg, 28 μmol) and Cs₂CO₃ (400 mg, 1.23 mmol) in dioxane (5 mL) was stirred in a sealed tube at 120° C. for 16 h. The mixture was cooled, diluted with EtOAc (30 mL) and filtered through diatomaceous earth and the filtrate was evaporated. The residue was purified by chromatography, eluting with a gradient (0-1.5%) of MeOH/DCM to give imidazopyridinone 225 (83 mg, 69%) as a yellow solid: mp 272-274° C.; ¹H NMR [(CD₃)₂SO] δ 8.93 (s, 1H, H-5″), 8.72 (d, J=1.9 Hz, 1H, H-3″), 8.63 (d, J=1.9 Hz, 1H, H-2″), 8.19 (s, 1H, NH), 8.11 (s, 1H, H-4), 7.85 (d, J=0.8 Hz, 1H, C-8″), 7.39 (s, 1H, H-7), 4.46 (tt, J=12.4, 4.1 Hz, 1H, H-4′), 4.04 (br dd, J=11.5, 4.1 Hz, 2 H, H-2′a, H-6′a), 3.51 (t, J=11.6 Hz, 2 H, H-2′b, H-6′b), 3.37 (s, 3H, NCH₃), 2.59 (s, 3H, 7-CH₃), 2.34 (ddd, J=25.0, 12.7, 4.6 Hz, 2 H, H-3′a, H-5′a), 1.72 (dd, J=12.4, 2.6 Hz, 2 H, H-3′b, H-5′b). HRMS calcd for C₂₁H₂₃N₆O₂ (MH⁺) m/z 391.1877, found 391.1881 (1.12 ppm). HPLC purity 96.1%

### Example 150: SN40528 Ethyl 7-methyl-6-((3-methyl-2-oxo-1-(tetrahydro-2H-pyran-4-yl)-2,3-dihydro-1H-imidazo[4,5-c]pyridin-6-yl)amino)imidazo[1,2-a]pyridine-2-carboxylate (226)

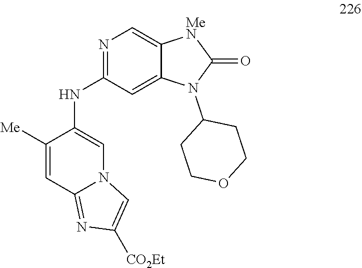 [SMILES: CCOC(=O)c1cn2cc(Nc3cc4c(cn3)n(C)c(=O)n4C3CCOCC3)c(C)cc2n1]

Ethyl 6-amino-7-methylimidazo[1,2-a]pyridine-2-carboxylate. To ethyl 7-methyl-6-nitroimidazo[1,2-a]pyridine-2-carboxylate (1.00 g, 4.01 mmol) in MeOH (30 mL) added 5% Pd/C (100 mg, 10% w/w) and the resulting slurry stirred under H₂ (1 atm.) for 12 h, filtered through a plug of diatomaceous earth, washing with MeOH and then solvent was removed in vacuo. The crude product was purified by chromatography eluting with a gradient (1-5%) MeOH/DCM to give ethyl 6-amino-7-methylimidazo[1,2-a]pyridine-2-carboxylate (170 mg, 19%) as a semi-pure brown solid which was used without further purification: ¹H NMR [(CD₃)₂SO] 8.28 (d, J=0.6 Hz, 1H, H-5), 7.72 (s, 1H, H-3), 7.25 (s, 1H, H-8), 4.89 (s, 2H, NH₂), 4.25 (q, J=7.1 Hz, 2 H, CH₂CH₃), 2.20 (d, J=0.8 Hz, 7-CCH₃), 1.29 (t, J=7.1 Hz, 3 H, CH₂CH₃).

Ethyl 7-methyl-6-((3-methyl-2-oxo-1-(tetrahydro-2H-pyran-4-yl)-2,3-dihydro-1H-imidazo[4,5-c]pyridin-6-yl)amino)imidazo[1,2-a]pyridine-2-carboxylate (226). A degassed mixture of chloride 120 (170 mg, 0.64 mmol), ethyl 6-amino-7-methylimidazo[1,2-a]pyridine-2-carboxylate (168 mg, 0.77 mmol), BrettPhos Pd(II) G3 (29 mg, 32 μmol) and Cs₂CO₃ (460 mg, 1.4 mmol) in dioxane (5 mL) was stirred in a sealed tube at 120° C. for 16 h. The mixture was cooled and the mixture diluted with EtOAc (30 mL) and filtered through diatomaceous earth and the filtrate was evaporated. The residue was purified by chromatography, eluting with a gradient (0.5-2%) of MeOH/DCM to give imidazopyridinone 226 (18 mg, 6%) as a brown solid: ¹H NMR [(CD₃)₂SO] δ 9.22 (s, 1H, H-5), 8.45 (s, 1H, H-3), 7.92 (H-4′), 7.87 (s, 1H, NH), 7.43 (s, 1H, H-8), 7.09 (s, 1H, H-7), 4.42 (tt, J=12.3, 4.2 Hz, 1H, H-4″), 4.28 (q, J=7.1 Hz, 2 H, OCH₂CH₃), 4.01 (dd, J=11.3, 4.1 Hz, 2 H, H-2″a, H-6″a), 3.49 (t, J=11.3 Hz, H-2″b, H-6″b), 3.31 (s, 3H, NCH₃), 2.37 (s, 3H, 7-CH₃), 2.36-2.24 (m, 2H, H-3″a, H-5″a), 1.69 (d, J=12.3, 2.7 Hz, H-3″b, H-5″b), 1.31 (t, J=7.1 Hz, OCH₂CH₃). HRMS calcd for C₂₃H₂₇N₆O₄ (MH⁺) m/z 451.2088, found 451.2092 (+0.85 ppm). HPLC purity 94.5%

### Example 151: SN40558 6-((4-(2-(Dimethylammonio)ethoxy)-2-methylphenyl)amino)-3-methyl-2-oxo-1-(tetrahydro-2H-pyran-4-yl)-2,3-dihydro-1H-imidazo[4,5-c]pyridin-5-ium dichloride (230)

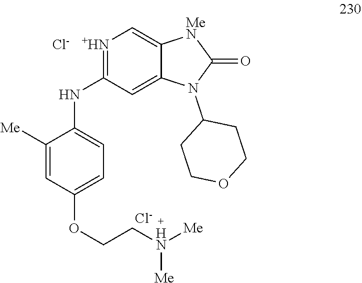

tert-Butyl (4-(benzyloxy)-2-methylphenyl)(3-methyl-2-oxo-1-(tetrahydro-2H-pyran-4-yl)-2,3-dihydro-1H-imidazo[4,5-c]pyridin-6-yl)carbamate (227). A mixture of aniline 124 (416 mg, 0.89 mmol), iPr₂NEt (0.23 mL, 1.34 mmol), DMAP (11 mg, 0.09 mmol) and BOC₂O (388 mg, 1.78 mmol) in dry THF (25 ml) was stirred at 70° C. for 16 h. The mixture was cooled to 20° C., diluted with EtOAc (100 mL) and washed with water (2×25 mL), brine (30 mL), dried (MgSO₄) and the solvent evaporated. The residue was purified by chromatography, eluting with a gradient (75-100%) of EtOAc/pet. ether, to give carbamate 227 (511 mg, 100%) as a white foam: ¹H NMR (CDCl₃) δ 7.93 (s, 1H, H-4′), 7.50 (s, 1H, H-7), 7.43 (br d, J=8.4 Hz, 2 H, H-2″″, H-6), 7.38 (ddd, J=8.2, 7.1, 1.5 Hz, 2 H, H-3″″, H-5), 7.32 (tt, J=7.1, 1.5 Hz, 1H, H-4), 7.09 (d, J=8.6 Hz, 1H, H-6″′), 6.88 (d, J=2.9 Hz, 1H, H-3″′), 6.80 (dd, J=8.6, 2.9 Hz, 1H, H-5″′), 5.04 (s, 2H, CH₂O), 4.54 (tt, J=12.4, 4.2 Hz, 1H, 1′-CH), 4.16 (br dd, J=11.6, 4.3 Hz, 2 H, H-2″, H-6″), 3.57 (br dt, J=11.9, 1.5 Hz, 2 H, H-2″, H-6″), 3.38 (s, 3H, 3′-CH₃), 2.47 (dq, J=12.6, 4.6 Hz, 2 H, H-3″, H-5″), 2.23 (s, 3H, 2″′-CH₃), 1.78 (br dd, J=12.4, 2.4 Hz, 2 H, H-3′, H-5′), 1.44 (s, 9H, CO₂tBu); MS m/z 545.2 (MH⁺, 100%); HRMS calcd for C₃₁H₃₇N₄O₅ (MH⁺) m/z 545.2758, found 545.2780 (−4.0 ppm).

tert-Butyl (4-hydroxy-2-methylphenyl)(3-methyl-2-oxo-1-(tetrahydro-2H-pyran-4-yl)-2,3-dihydro-1H-imidazo[4,5-c]pyridin-6-yl)carbamate (228). A mixture of benzyl ether 227 (498 mg, 0.91 mmol) and Pd/C (50 mg) in EtOH (50 mL) was stirred under H₂ (50 psi) at 20° C. for 16 h. The mixture was filtered through diatomaceous earth and the filtrate was evaporated. The residue was purified by chromatography, eluting with a gradient (50-100%) of EtOAc/pet. ether, to give phenol 228 (402 mg, 97%) as a white powder: mp 223° C. (dec.); ¹H NMR (CDCl₃) δ 7.93 (s, 1H, H-4′), 7.52 (s, 1H, H-7), 6.97 (dd, J=6.4, 2.7 Hz, 1 H, H-5″′), 6.53-6.57 (m, 2H, H-3″′, H-6″′), 6.07 (s, 1H, 4″′—OH), 4.54 (tt, J=12.4, 4.3 Hz, 1 H, 1′-CH), 4.16 (br dd, J=11.5, 4.4 Hz, 2 H, H-2″, H-6″), 3.58 (br dt, J=12.0, 1.5 Hz, 2 H, H-2″, H-6″), 3.39 (s, 3H, 3′-CH₃), 2.47 (dq, J=12.5, 4.6 Hz, 2 H, H-3″, H-5″), 2.17 (s, 3H, 2″′-CH₃), 1.80 (br dd, J=12.4, 2.4 Hz, 2 H, H-3′, H-5′), 1.44 (s, 9H, CO₂tBu); MS m/z 455.2 (MH⁺, 100%); HRMS calcd for C₂₄H₃1N₄O₅ (MH⁺) m/z 455.2302, found 455.2314 (−2.5 ppm).

tert-Butyl (4-(2-(dimethylamino)ethoxy)-2-methylphenyl)(3-methyl-2-oxo-1-(tetrahydro-2H-pyran-4-yl)-2,3-dihydro-1H-imidazo[4,5-c]pyridin-6-yl)carbamate (229). To phenol 228 (500 mg, 1.10 mmol) in DMF (10 mL) added 2-chloro-N,N-dimethylethan-1-aminium chloride (170 mg, 1.21 mmol) and Cs₂CO₃ (1.08 g, 3.30 mmol) and the mixture was stirred at 40-50° C. for 18 h, diluted with water (50 mL), extracted with EtOAc (2×25 mL), and the combined organic fractions washed with water (3×20 mL), dried and solvent removed in vacuo. The crude residue was purified by chromatography, eluting with 5% MeOH/DCM to give 229 (390 mg, 67%) as a brown foam.

6-((4-(2-(Dimethylammonio)ethoxy)-2-methylphenyl)amino)-3-methyl-2-oxo-1-(tetrahydro-2H-pyran-4-yl)-2,3-dihydro-1H-imidazo[4,5-c]pyridin-5-ium chloride (230). To 229 (45 mg, 0.038 mmol) was added 4 M HCl in dioxane (1 mL) and the mixture was stirred for 5 h, then solvent was removed in vacuo to give imidazopyridinone 230 (49 mg, quant.) as a brown solid: ¹H NMR [(CD₃)₂SO] δ 12.96 (br s, 1H, NH), 10.88 (br s, 1H, NH), 9.72 (br s, 1H, NH), 7.80 (s, 1H, H-4), 7.27 (d, J=8.4 Hz, 1H, H-6″), 7.06 (s, 1H, H-3″), 6.97 (br d, J=7.6 Hz, H-5″), 6.84 (s, 1H, H-7), 4.49-4.35 (m, 3H, H-4′, OCH₂CH₂N), 3.97 (br d, 2 H, 7.8 Hz, H-2′a, H-6′a), 3.64-3.37 (m, 4H, H-2′b, H-6′b, OCH₂CH₂N), 3.30 (s, 3H, 3-NCH₃), 2.84 (d, J=4.0 Hz, 6 H, N(CH₃)₂), 2.32-2.14 (m, 5H, H-3′a, H-5′a, 2″-CCH₃), 1.71 (d, J=10.9 Hz, 2 H, H-3′b, H-5′b). HRMS calcd for C₂₃H₃₂N₅O₃(MH⁺) m/z 426.2500, found 426.2503 (+0.79 ppm). HPLC purity 99.8%

### Example 152: SN39586 1-Cyclopentyl-3-methyl-6-((2-methyl-4-((1-methyl-2-nitro-1H-imidazol-5-yl)methoxy)phenyl)amino)-1,3-dihydro-2H-imidazo[4,5-c]pyridin-2-one (234)

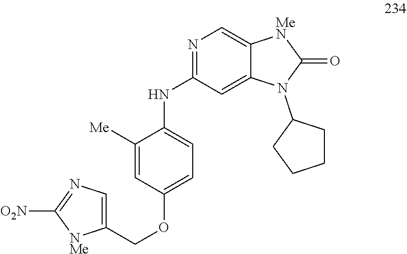 [SMILES: Cc1cc(OCc2cnc([N+](=O)[O-])n2C)ccc1Nc1cc2c(cn1)n(C)c(=O)n2C1CCCC1]

tert-Butyl (4-(benzyloxy)-2-methylphenyl)(1-cyclopentyl-3-methyl-2-oxo-2,3-dihydro-1H-imidazo[4,5-c]pyridin-6-yl)carbamate (231). A mixture of aniline 51 (454 mg, 1.06 mmol), iPr₂NEt (0.28 mL, 1.59 mmol), DMAP (13 mg, 0.11 mmol) and BOC₂O (347 mg, 1.59 mmol) in dry THF (25 ml) was stirred at 70° C. for 16 h. The mixture was cooled to 20° C., diluted with EtOAc (100 mL) and washed with water (2×25 mL), brine (30 mL), dried (MgSO₄) and the solvent evaporated. The residue was purified by chromatography, eluting with a gradient (40-70%) of EtOAc/pet. ether, to give carbamate 231 (512 mg, 91%) as a tan foam: ¹H NMR (CDCl₃) δ 7.76 (s, 1H, H-4), 7.36-7.45 (m, 4H, H-2″, H-3″, H-5″, H-6″), 7.29-7.34 (m, 2H, H-7, H-4″), 7.08 (d, J=8.6 Hz, 1H, H-6′), 6.87 (d, J=2.9 Hz, 1H, H-3′), 6.79 (dd, J=8.6, 2.9 Hz, 1H, H-5′), 5.04 (s, 2H, CH₂O), 4.78 (pent, J=8.7 Hz, 1H, 1-CH), 3.37 (s, 3H, 3-CH₃), 2.23 (s, 3H, 2′-CH₃), 2.02-2.10 (m, 4H, 2×CH₂), 1.93-2.00 (m, 2H, CH₂), 1.70-1.78 (m, 2H, CH₂), 1.43 (s, 9H, CO₂tBu); MS m/z 529.2 (MH⁺, 100%).

tert-Butyl (1-cyclopentyl-3-methyl-2-oxo-2,3-dihydro-1H-imidazo[4,5-c]pyridin-6-yl)(4-hydroxy-2-methylphenyl)carbamate (232). A mixture of benzyl ether 231 (495 mg, 0.94 mmol) and Pd/C (50 mg) in EtOH (50 mL) was stirred under Hz (50 psi) at 20° C. for 16 h. The mixture was filtered through diatomaceous earth and the filtrate was evaporated. The residue was purified by chromatography, eluting with a gradient (50-100%) of EtOAc/pet. ether, to give phenol 232 (429 mg, 100%) as a clear gum: ¹H NMR (CDCl₃) δ 7.91 (s, 1H, H-4″), 7.57 (s, 1H, H-7), 6.92 (d, J=8.4 Hz, 1H, H-6′), 6.73 (br s, 1H, 4′-OH), 6.46 (m, 2H, H-3′, H-5′), 4.81 (pent, J=8.7 Hz, 1H, 1″-CH), 3.39 (s, 3H, 3″-CH₃), 2.15 (s, 3H, 2′-CH₃), 2.05-2.32 (m, 4H, 2×CH₂), 1.93-2.02 (m, 2H, CH₂), 1.74-1.80 (m, 2H, CH₂), 1.44 (s, 9H, CO₂tBu); MS m/z 439.2 (MH⁺, 100%). HRMS calcd for C₂₄H₃1N₄O₄ (MH⁺) m/z 439.2353, found 439.2358 (−1.1 ppm).

tert-Butyl (1-cyclopentyl-3-methyl-2-oxo-2,3-dihydro-1H-imidazo[4,5-c]pyridin-6-yl)(2-methyl-4-((1-methyl-2-nitro-1H-imidazol-5-yl)methoxy)phenyl)carbamate (233). A mixture of phenol 232 (422 mg, 0.96 mmol), 5-(bromomethyl)-1-methyl-2-nitro-1H-imidazole² (255 mg, 1.15 mmol) and Cs₂CO₃ (407 mg, 1.25 mmol) in dry DMF (20 mL) was stirred at 60° C. for 3 h. The mixture was cooled and diluted with EtOAc (100 mL). The organic fraction was washed with water (3×50 mL), washed with brine (30 mL), dried (MgSO₄), filtered and the solvent evaporated. The residue was purified by chromatography, eluting with a gradient (80-100%) of EtOAc/pet. ether, to give carbamate 233 (554 mg, 100%) as a clear oil: ¹H NMR (CDCl₃) δ 7.89 (s, 1H, H-4″′), 7.39 (s, 1H, H-4″), 7.21 (s, 1H, H-7″′), 7.13 (d, J=8.6 Hz, 1H, H-6′), 6.85 (d, J=2.9 Hz, 1H, H-3′), 6.79 (dd, J=8.6, 2.9 Hz, 1H, H-5′), 5.04 (s, 2H, CH₂O), 4.81 (pent, J=8.8 Hz, 1H, 1″′-CH), 4.06 (s, 3H, 1″-CH₃), 3.55 (s, 3H, 3″′-CH₃), 2.25 (s, 3H, 2″-CH₃), 2.05-2.12 (m, 4H, 2×CH₂), 1.94-2.02 (m, 2H, CH₂), 1.72-1.79 (m, 2H, CH₂), 1.44 (s, 9H, CO₂tBu); MS m/z 578.2 (MH⁺, 100%). HRMS calcd for C₂₉H₃₆N₇O₆ (MH⁺) m/z 578.2748, found 578.2743 (0.9 ppm).

1-Cyclopentyl-3-methyl-6-((2-methyl-4-((1-methyl-2-nitro-1H-imidazol-5-yl)methoxy)phenyl)amino)-1,3-dihydro-2H-imidazo[4,5-c]pyridin-2-one (234). TFA (1.5 mL, 19.4 mmol) was added to a stirred solution of carbamate 233 (0.56 g, 0.97 mmol) in DCM (10 mL) and the mixture was stirred at 20° C. for 24 h. The solvent was evaporated and the residue was partitioned between EtOAc (50 ml) and aqueous NaHCO₃ (50 mL). The organic fraction was washed with water (30 mL), washed with brine (30 mL), dried (MgSO₄), filtered and the solvent evaporated. The residue was purified by chromatography, eluting with a gradient (0-3%) of MeOH/DCM, to give imidazopyridinone 234 (354 mg, 76%) as an orange solid: mp 168-170° C.; ¹H NMR (CDCl₃) δ 7.77 (s, 1H, H-4), 7.32 (d, J=8.6 Hz, 1H, H-6′), 7.23 (s, 1H, H-4″), 6.89 (d, J=2.9 Hz, 1H, H-3′), 6.83 (dd, J=8.6, 2.9 Hz, 1H, H-5′), 6.21 (d, J=0.4 Hz, 1H, H-7), 5.98 (br s, 1H, 6-NH), 5.06 (s, 2H, CH₂O), 4.71 (pent, J=8.8 Hz, 1 H, 1-CH), 4.09 (s, 3H, H-1″), 3.38 (s, 3H, 3-CH₃), 2.27 (s, 3H, 2′-CH₃), 1.92-1.99 (m, 4H, 2×CH₂), 1.73-1.82 (m, 2H, CH₂), 1.61-1.68 (m, 2H, CH₂); MS m/z 478.2 (MH⁺, 100%); HRMS calcd for C₂₄H₂₈N₇₀₄ (MH⁺) m/z 478.2197, found 478.2206 (−1.9 ppm). HPLC purity 99.4%.

### Example 153: SN39897 3-Methyl-6-((2-methyl-4-((1-methyl-2-nitro-1H-imidazol-5-yl)methoxy)phenyl)amino)-1-(tetrahydro-2H-pyran-4-yl)-1,3-dihydro-2H-imidazo[4,5-c]pyridin-2-one (236)

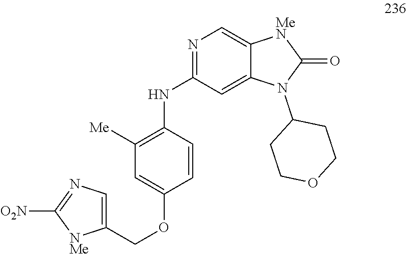 [SMILES: Cc1cc(OCc2cnc([N+](=O)[O-])n2C)ccc1Nc1cc2c(cn1)n(C)c(=O)n2C1CCOCC1]

tert-Butyl (3-methyl-2-oxo-1-(tetrahydro-2H-pyran-4-yl)-2,3-dihydro-1H-imidazo[4,5-c]pyridin-6-yl)(2-methyl-4-((1-methyl-2-nitro-1H-imidazol-5-yl)methoxy)phenyl)carbamate (235). A mixture of phenol 228 (395 mg, 0.87 mmol), 5-(chloromethyl)-1-methyl-2-nitro-1H-imidazole³ (168 mg, 0.96 mmol) and Cs₂CO₃ (369 mg, 1.13 mmol) in dry DMF (20 mL) was stirred at 60° C. for 3 h. The mixture was cooled and diluted with EtOAc (100 mL). The organic fraction was washed with water (3×50 mL), washed with brine (30 mL), dried (MgSO₄), filtered and the solvent evaporated. The residue was purified by chromatography, eluting with a gradient (80-100%) of EtOAc/pet. ether, to give carbamate 235 (185 mg, 36%) as a clear oil: ¹H NMR (CDCl₃) δ 7.91 (s, 1H, H-4), 7.54 (s, 1H, H-4″), 7.21 (s, 1H, H-7″′), 7.15 (d, J=8.6 Hz, 1H, H-6′), 6.87 (d, J=2.9 Hz, 1H, H-3), 6.80 (dd, J=8.6, 2.9 Hz, 1H, H-5′), 5.04 (s, 2H, CH₂O), 4.54 (tt, J=12.4, 4.2 Hz, 1H, 1″′-CH), 4.18 (br dd, J=11.6, 4.2 Hz, 2 H, H-2″″, H-6), 4.05 (s, 3H, 1″′-CH₃), 3.58 (br t, J=11.9 Hz, 2 H, H-2″″, H-6), 3.38 (s, 3H, 3″′-CH₃), 2.48 (dq, J=12.6, 4.6 Hz, 2 H, H-3″′, H-5″′), 2.17 (s, 3H, 2″′-CH₃), 1.80 (br dd, J=12.6, 2.6 Hz, 2 H, H-3″″, H-5), 1.43 (s, 9H, CO₂tBu); MS m/z 594.2 (MH⁺, 100%).

3-Methyl-6-((2-methyl-4-((1-methyl-2-nitro-1H-imidazol-5-yl)methoxy)phenyl)amino)-1-(tetrahydro-2H-pyran-4-yl)-1,3-dihydro-2H-imidazo[4,5-c]pyridin-2-one (236). TFA (0.5 mL, 6.2 mmol) was added to a stirred solution of carbamate 235 (185 mg, 0.31 mmol) in DCM (10 mL) and the mixture was stirred at 20° C. for 24 h. The solvent was evaporated and the residue was partitioned between EtOAc (50 mL) and aqueous NaHCO₃(50 mL). The organic fraction was washed with water (30 mL), washed with brine (30 mL), dried (MgSO₄), filtered and the solvent evaporated. The residue was purified by chromatography, eluting with a gradient (0-5%) of MeOH/DCM, to give imidazopyridinone 236 (65 mg, 42%) as an clear glass: ¹H NMR (CDCl₃) δ 7.78 (s, 1H, H-4), 7.33 (d, J=8.7 Hz, 1H, H-6″), 7.24 (s, 1H, H-4), 6.90 (d, J=2.9 Hz, 1H, H-3″), 6.82 (dd, J=8.7, 2.8 Hz, 1H, H-5″), 6.33 (s, 1H, H-7), 6.14 (br s, 1H, 6-NH), 5.07 (s, 2H, CH₂O), 4.49 (tt, J=12.4, 4.2 Hz, 1H, 1-CH), 4.03-4.15 (m, 5H, H-2′, H-6′, 1″′-CH₃), 3.51 (br t, J=11.9 Hz, 2 H, H-2′, H-6′), 3.40 (s, 3H, 3-CH₃), 2.20-2.32 (m, 5H, H-3′, H-5′, 2″′-CH₃), 1.72 (br dd, J=12.4, 2.5 Hz, 2 H, H-3′, H-5′); MS m/z 494.2 (MH⁺, 100%); HRMS calcd for C₂₄H₂₈N₇O₅ (MH⁺) m/z 494.2416, found 494.2151 (−0.9 ppm). HPLC purity 99.4%.

### Example 154: SN40458 3-Methyl-6-((2-methyl-4-(1-(1-methyl-2-nitro-1H-imidazol-5-yl)ethoxy)phenyl)amino)-1-(tetrahydro-2H-pyran-4-yl)-1,3-dihydro-2H-imidazo[4,5-c]pyridin-2-one (238)

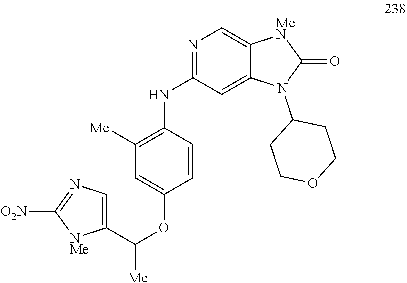 [SMILES: Cc1cc(OC(C)c2cnc([N+](=O)[O-])n2C)ccc1Nc1cc2c(cn1)n(C)c(=O)n2C1CCOCC1]

tert-Butyl (3-methyl-2-oxo-1-(tetrahydro-2H-pyran-4-yl)-2,3-dihydro-1H-imidazo[4,5-c]pyridin-6-yl)(2-methyl-4-(1-(1-methyl-2-nitro-1H-imidazol-5-yl)ethoxy)phenyl)carbamate (237). To phenol 228 (180 mg, 0.40 mmol) in DMF (2 mL) was added 5-(1-chloroethyl)-1-methyl-2-nitro-1H-imidazole (83 mg, 0.44 mmol) in DMF (1 mL) and Cs₂CO₃ (170 mg, 0.52 mmol) and the mixture was stirred 18 h, cooled to 0° C. and diluted with water (5 mL). The aqueous mixture was extracted with EtOAc (3×5 mL), the combined organic fractions were washed with water (2×10 mL), dried over Na₂SO₄ and solvent was removed in vacuo. The resulting residue was purified by chromatography, eluting with EtOAc to give carbamate 237 (240 mg, quant.): ¹H NMR (CDCl₃) δ 7.91 (s, 1H, H-4), 7.52 (s, 1H, H-7), 7.18 (s, 1H, H-4″′), 7.11 (d, J=8.6 Hz, 1H, H-6″), 6.82 (d, J=2.8 Hz, 1 H, H-3″), 6.76 (dd, J=8.8, 2.9 Hz, 1H, H-5″), 5.43 (q, J=6.5 Hz, 1H, 5″′-CCH(CH₃)), 4.56 (tt, J=12.5, 4.2 Hz, 1H, H-4′), 4.17 (dd, J=11.6, 4.2 Hz, 2 H, H-2′a, H-6′a), 4.01 (1″′-NCH₃), 3.58 (td, J=12.0, 1.5 Hz, 2 H, H-2′b, H-6′b), 3.39 (s, 3H, 3-NCH₃), 2.46 (app. qd, J=12.6, 4.6 Hz, H-3′a, H-5′a), 2.23 (s, 3H, 2″-CCH₃), 1.84-1.74 (5H, m, 5″′-CCH(CH₃), H-3′b, H-5′b), 1.44 (s, 9H, N(O)OC(CH₃)₃).

3-Methyl-6-((2-methyl-4-(1-(1-methyl-2-nitro-1H-imidazol-5-yl)ethoxy)phenyl)amino)-1-(tetrahydro-2H-pyran-4-yl)-1,3-dihydro-2H-imidazo[4,5-c]pyridin-2-one (238). To carbamate 237 (200 mg, 0.33 mmol) in DCM (10 mL) was added TFA (0.51 mL, 6.6 mmol) and the resulting mixture was stirred for 24 h, solvent removed in vacuo and the residue purified by chromatography, eluting with 100% EtOAc, to give ether 238 (100 mg, 59%) as a yellow solid: mp 229-231° C.; ¹H NMR [(CD₃)₂SO] δ 7.78 (s, 1H, H-4), 7.68 (s, 1H, NH), 7.55 (d, J=8.8 Hz, 1H, H-6″), 7.27 (s, 1H, H-4″′), 6.90 (d, J=2.8 Hz, 1 H, H-3″), 6.82 (dd, J=8.8, 2.9 Hz, 1H, H-5″), 6.75 (s, 1H, H-7), 5.68 (q, J=6.4 Hz, 1H, 5″′-CCH), 4.37 (tt, J=12.3, 4.2 Hz, 1H, H-4′), 3.99 (dd, J=11.4, 4.1, 2 H, H-2′a, H-6′a), 3.95 (s, 3H, 1″′-NCH₃), 3.46 (t, J=11.4 Hz, 2 H, H-2′b, H-6′b), 3.27 (s, 3H, 3-NCH₃), 2.31-2.18 (m, 2H, H-3′a, H-5′a), 2.19 (s, 3H, 2″-CCH₃), 1.70-1.61 (m, 2H, H-3′b, H-5′b), 1.64 (d, J=6.4 Hz, 3 H, 5″′-CCH(CH₃)). HRMS calcd for C₂₅H₃₀N₇O₅(MH⁺) m/z 508.2303, 508.2314 (+2.15 ppm). HPLC purity 99.9%

### Example 155: SN40459 3-Methyl-6-((2-methyl-4-((1-methyl-5-nitro-1H-imidazol-2-yl)methoxy)phenyl)amino)-1-(tetrahydro-2H-pyran-4-yl)-1,3-dihydro-2H-imidazo[4,5-c]pyridin-2-one (240)

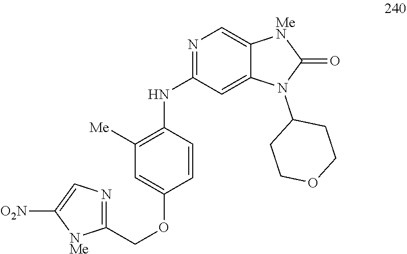 [SMILES: Cc1cc(OCc2ncc([N+](=O)[O-])n2C)ccc1Nc1cc2c(cn1)n(C)c(=O)n2C1CCOCC1]

tert-Butyl (3-methyl-2-oxo-1-(tetrahydro-2H-pyran-4-yl)-2,3-dihydro-1H-imidazo[4,5-c]pyridin-6-yl)(2-methyl-4-((1-methyl-5-nitro-1H-imidazol-2-yl)methoxy)phenyl)carbamate (239). To phenol 228 (200 mg, 0.44 mmol) in DMF (3 mL) was added (1-methyl-5-nitro-1H-imidazol-2-yl)methanol (102 mg, 0.57 mmol) and Cs₂CO₃ (190 mg, 0.57 mmol) and the mixture was stirred 24 h, cooled to 0° C. and diluted with water (5 mL). The aqueous mixture was extracted with EtOAc (3×5 mL), the combined organic fractions were washed with water (2×10 mL), dried over Na₂SO₄ and solvent was removed in vacuo. The resulting residue was purified by chromatography, eluting with a gradient of (50-100%) EtOAc/pet. ether to give carbamate 239 (190 mg, 73%): ¹H NMR (CDCl₃) δ 7.97 (s, 1H, H-4″′), 7.91 (s, 1H, H-4), 7.52 (s, 1H, H-7), 7.11 (d, J=8.6 Hz, 1H, H-6″), 6.89 (d, J=2.9, 1 H, H-3″), 6.84 (dd, J=8.6, 3.0 Hz, 1H, H-5″), 5.19 (s, 2H, 2″′-CCH₂), 4.55 (tt, J=12.4, 4.3 Hz, 1H, H-4′), 4.16 (dd, J=11.6, 4.3 Hz, 2 H, H-2′a, H-6′a), 4.06 (s, 3H, 1″′-NCH₃), 3.57 (td, J=11.9, 1.5 Hz, 2 H, H-2′b, H-6′b), 3.38 (s, 3H, 3-NCH₃), 2.47 (app. qd, J=12.6, 4.5 Hz, 2 H, H-3′a, H-5′a), 2.24 (s, 3H, 2″-CCH₃), 1.79 (dd, J=12.4, 2.6 Hz, 2 H, H-3′b, H-5′b), 1.44 (s, 9H, N(O)OC(CH₃)₃).

3-Methyl-6-((2-methyl-4-((1-methyl-5-nitro-1H-imidazol-2-yl)methoxy)phenyl)amino)-1-(tetrahydro-2H-pyran-4-yl)-1,3-dihydro-2H-imidazo[4,5-c]pyridin-2-one (240). To carbamate 239 (150 mg, 0.25 mmol) in DCM (8 mL) was added TFA (0.38 mL, 5.0 mmol) and the resulting mixture was stirred for 24 h, solvent removed in vacuo and the residue taken up in EtOAc (10 mL), washed with saturated NaHCO₃(10 mL), water (10 mL), saturated NaCl (10 mL) and dried over Na₂SO₄. Solvent was removed in vacuo and the residue was purified by chromatography, eluting with 100% EtOAc, to give ether 240 (110 mg, 92%) as a yellow solid: mp 196-198° C.; ¹H NMR [(CD₃)₂SO] δ 8.08 (s, 1H, H-4″′), 7.79 (s, 1H, H-4), 7.70 (s, 1H, NH), 7.55 (d, J=8.8 Hz, 1H, H-6″), 6.93 (d, J=2.9 Hz, 1H, H-3″), 6.85 (dd, J=8.8, 3.0 Hz, 1H, H-5″), 6.74 (s, 1H, H-7), 5.23 (s, 2H, 2″′-CCH₂), 4.37 (tt, J=12.3, 4.2 Hz, 1H, H-4′), 4.03-3.94 (m, 2H, H-2′a, H-6′a), 3.96 (s, 3H, 1″′-NCH₃), 3.46 (t, J=11.3 Hz, H-2′b, H-6′b), 3.27 (s, 3H, 3-NCH₃), 2.32-2.16 (m, 2H, H-3′a, H-5′a), 2.19 (s, 3H, 2″-CCH₃), 1.66 (dd, J=12.2, 2.2 Hz, H-3′b, H-5′b). HRMS calcd for C₂₄H₂₈N₇O₅ (MH⁺) m/z 494.2146, found 494.2155 (+1.67 ppm). HPLC purity 99.9%

### Example 156: SN40588 3-Methyl-6-((2-methyl-4-((5-nitrothiophen-2-yl)methoxy)phenyl)amino)-1-(tetrahydro-2H-pyran-4-yl)-1,3-dihydro-2H-imidazo[4,5-c]pyridin-2-one (242)

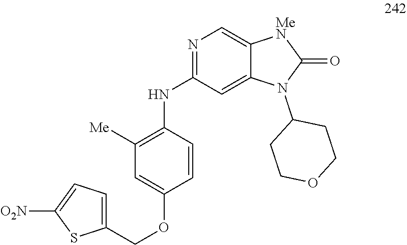 [SMILES: Cc1cc(OCc2ccc([N+](=O)[O-])s2)ccc1Nc1cc2c(cn1)n(C)c(=O)n2C1CCOCC1]

tert-Butyl (3-methyl-2-oxo-1-(tetrahydro-2H-pyran-4-yl)-2,3-dihydro-1H-imidazo[4,5-c]pyridin-6-yl)(2-methyl-4-((5-nitrothiophen-2-yl)methoxy)phenyl)carbamate (241). To polymer-supported triphenylphosphine (150 mg, 3 mmol/g, 0.44 mmol) in THF (2 mL) at 0° C. was added diethyl azodicarboxylate (86 μL, 0.44 mmol) dropwise and the resulting solution was stirred at 0° C. for 10 minutes. Phenol 228 (100 mg, 0.22 mmol) and (5-nitrothiophen-2-yl)methanol (37 mg, 0.23 mmol) were combined in THF (2 mL) and this resulting mixture was added dropwise to the triphenylphospine, diethyl azodicarboxylate mixture then the resulting mixture was stirred for 24 hours, volatiles removed in vacuo and the resulting residue purified by chromatography, eluting with a gradient (0-50%) EtOAc/DCM to give carbamate 241 (60 mg, 46%): MS (M+H) 596.0; 1H NMR [(CD₃)₂SO]δ 8.07 (d, J=4.2 Hz, 1H, H-4), 8.01 (s, 1H, H-4′), 7.56 (1H, s, H-7), 7.30 (d, J=4.2 Hz, 1 H, H-3), 7.12 (d, J=8.6 Hz, 1H, H-6″′), 6.95 (d, J=2.8 Hz, 1H, H-3″′), 6.83 (dd, J=8.6, 2.9 Hz, 1H, H-5″′), 5.39 (s, 2H, 2-CCH₂), 4.48 (tt, J=12.2, 4.2 Hz, 1H, H-4″), 4.00 (d, J=11.8, 4.2 Hz, 2 H, H-2′a, H-6′a), 3.50 (t, J=11.3 Hz, 2 H, H-2′b, H-6′b), 3.30 (s, 3H, 3′-NCH₃), 2.43-2.28 (m, 2H, H-3′a, H-5′a), 2.20 (s, 3H, 2″′-CCH₃), 1.68 (br d, J=9.9 Hz, 2 H, H-3′b, H-5′b), 1.37 (s, 9H, OC(CH₃)₃).

3-Methyl-6-((2-methyl-4-((5-nitrothiophen-2-yl)methoxy)phenyl)amino)-1-(tetrahydro-2H-pyran-4-yl)-1,3-dihydro-2H-imidazo[4,5-c]pyridin-2-one (242) To carbamate 241 (60 mg, 0.1 mmol) in DCM (1 mL) was added TFA (1 mL) and the resulting mixture was stirred for 5 h, solvent removed in vacuo and the residue taken up in DCM (10 mL), washed with saturated NaHCO₃(2×10 mL), saturated NaCl (1×10 mL), dried over Na₂SO₄ and solvent removed in vacuo to give imidazopyridinone 242: MS (M+H) 496.0.

### Example 157: SN39591 1-(4-Methoxyphenyl)-3-methyl-6-((2-methyl-4-((1-methyl-2-nitro-1H-imidazol-5-yl)methoxy)phenyl)amino)-1,3-dihydro-2H-imidazo[4,5-c]pyridin-2-one (246)

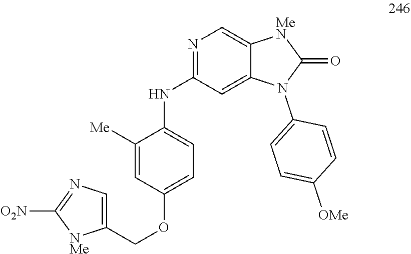 [SMILES: COc1ccc(-n2c(=O)n(C)c3cnc(Nc4ccc(OCc5cnc([N+](=O)[O-])n5C)cc4C)cc32)cc1]

tert-Butyl (4-(Benzyloxy)-2-methylphenyl)(1-(4-methoxyphenyl)-3-methyl-2-oxo-2,3-dihydro-1H-imidazo[4,5-c]pyridin-6-yl)carbamate (243). A mixture of aniline 199 (338 mg, 0.72 mmol), iPr₂NEt (0.19 mL, 1.09 mmol), DMAP (9 mg, 0.07 mmol) and BOC₂O (235 mg, 1.09 mmol) in dry THF (25 ml) was stirred at 70° C. for 16 h. The mixture was cooled to 20° C., diluted with EtOAc (100 mL) and washed with water (2×25 mL), brine (30 mL), dried (MgSO₄) and the solvent evaporated. The residue was purified by chromatography, eluting with a gradient (50-80%) of EtOAc/pet. ether, to give carbamate 243 (381 mg, 93%) as a pink foam: ¹H NMR (CDCl₃) δ 7.98 (d, J=0.4 Hz, 1H, H-4′), 7.35-7.45 (m, 6H, H-2″, H-6″, H-2, H-3, H-5, H-6), 7.30-7.34 (m, 1H, H-4), 7.28 (s, 1H, H-7), 7.03-7.08 (m, 3H, H-3″, H-5″, H-6″′), 6.86 (d, J=2.8 Hz, 1H, H-3″′), 6.78 (dd, J=8.6, 2.8 Hz, 1H, H-5″′), 5.03 (s, 2H, CH₂O), 3.88 (s, 3H, 4″-OCH₃), 3.46 (s, 3H, 3′-CH₃), 2.20 (s, 3H, 2″′-CH₃), 1.39 (s, 9H, CO₂tBu); MS m/z 567.2 (MH⁺, 100%); HRMS calcd for C₃₃H₃₅N₄O₅ (MH⁺) m/z 567.2615, found 567.2627 (−2.1 ppm).

tert-Butyl (4-Hydroxy-2-methylphenyl)(1-(4-methoxyphenyl)-3-methyl-2-oxo-2,3-dihydro-1H-imidazo[4,5-c]pyridin-6-yl)carbamate (244). A mixture of benzyl ether 243 (380 mg, 0.67 mmol) and Pd/C (30 mg) in EtOH (25 mL) and EtOAc (25 mL) was stirred under Hz (50 psi) at 20° C. for 6 h. The mixture was filtered through diatomaceous earth and the filtrate was evaporated to give phenol 244 (320 mg, 100%) as a white powder: mp 148-151° C.; ¹H NMR (CDCl₃) δ 7.98 (d, J=0.6 Hz, 1H, H-4′), 7.41 (ddd, J=9.0, 3.2, 2.2 Hz, 2 H, H-2″, H-6″), 7.30 (s, 1H, H-7), 7.07 (ddd, J=9.0, 3.2, 2.2 Hz, 2 H, H-3″, H-5″), 6.91 (m, 1H, H-5″′), 6.48-6.53 (m, 2H, H-3″′, H-6″′), 6.30 (br s, 1H, 4″′—OH), 3.88 (s, 3H, 4″-OCH₃), 3.47 (s, 3H, 3′-CH₃), 2.12 (s, 3H, 2″′-CH₃), 1.39 (s, 9H, CO₂tBu); MS m/z 477.2 (MH⁺, 100%); HRMS calcd for C₂₆H₂₉N₄O₅ (MH⁺) m/z 477.2146, found 477.2139 (1.5 ppm).

tert-Butyl (1-(4-Methoxyphenyl)-3-methyl-2-oxo-2,3-dihydro-1H-imidazo[4,5-c]pyridin-6-yl)(2-methyl-4-((1-methyl-2-nitro-1H-imidazol-5-yl)methoxy)phenyl)carbamate (245). A mixture of phenol 244 (308 mg, 0.65 mmol), 5-(bromomethyl)-1-methyl-2-nitro-1H-imidazole² (156 mg, 0.71 mmol) and Cs₂CO₃ (254 mg, 0.78 mmol) in dry DMF (10 mL) was stirred at 60° C. for 3 h. The mixture was cooled and diluted with EtOAc (100 mL). The organic fraction was washed with water (3×50 mL), washed with brine (30 mL), dried (MgSO₄), filtered and the solvent evaporated. The residue was purified by chromatography, eluting with a gradient (80-100%) of EtOAc/pet. ether, to give carbamate 245 (210 mg, 52%) as a clear gum: ¹H NMR (CDCl₃) δ 7.96 (s, 1H, H-4), 7.41 (ddd, J=9.0, 3.4, 2.2 Hz, 2 H, H-2″″, H-6), 7.31 (s, 1H, H-4″), 7.20 (s, 1H, H-7″′), 7.10 (d, J=8.6 Hz, 1H, H-6′), 7.07 (ddd, J=9.0, 3.4, 2.2 Hz, 2 H, H-3, H-5), 6.84 (d, J=2.9 Hz, 1 H, H-3′), 6.78 (dd, J=8.6, 2.9 Hz, 1H, H-5′), 5.03 (s, 2H, CH₂O), 4.06 (s, 3H, 1″-CH₃), 3.89 (s, 3H, 4″″-OCH₃), 3.46 (s, 3H, 3″′-CH₃), 2.21 (s, 3H, 2′-CH₃), 1.39 (s, 9H, CO₂tBu); MS m/z 616.2 (MH⁺, 100%); HRMS calcd for C₃₁H₃₄N₇O₇ (MH⁺) m/z 616.2528, found 616.2500 (1.2 ppm).

1-(4-Methoxyphenyl)-3-methyl-6-((2-methyl-4-((1-methyl-2-nitro-1H-imidazol-5-yl)methoxy)phenyl)amino)-1,3-dihydro-2H-imidazo[4,5-c]pyridin-2-one (246). TFA (0.5 mL, 6.66 mmol) was added to a stirred solution of carbamate 245 (210 mg, 0.33 mmol) in DCM (10 mL) and the mixture was stirred at 20° C. for 24 h. The solvent was evaporated and the residue was partitioned between EtOAc (50 ml) and aqueous NaHCO₃ (50 mL). The organic fraction was washed with water (30 mL), washed with brine (30 mL), dried (MgSO₄), filtered and the solvent evaporated. The residue was purified by chromatography, eluting with a gradient (0-3%) of MeOH/DCM, to give imidazopyridinone 246 (175 mg, 100%) as a yellow powder: mp (EtOAc/pet. ether) 244-247° C.; ¹H NMR (CDCl₃) δ 7.84 (d, J=0.6 Hz, 1H, H-4), 7.31 (ddd, J=9.0, 3.3, 2.2 Hz, 2 H, H-2″′, H-6″′), 7.28 (d, J=8.6 Hz, 1H, H-6′), 7.20 (s, 1H, H-4″), 6.99 (ddd, J=9.0, 3.3, 2.2 Hz, 2 H, H-3″′, H-5″′), 6.83 (d, J=2.9 Hz, 1H, H-3), 6.75 (dd, J=8.6, 2.9 Hz, 1H, H-5′), 6.15 (d, J=0.6 Hz, 1H, H-7), 5.95 (br s, 1H, 6-NH), 5.02 (s, 2H, CH₂O), 4.07 (s, 3H, 1″-CH₃), 3.85 (s, 3H, 4″′—OCH₃), 3.48 (s, 3H, 3-CH₃), 2.23 (s, 3H, 2′-CH₃); MS m/z 516.2 (MH⁺, 100%); HRMS calcd for C₂₆H₂₆N₇O₅ (MH⁺) m/z 516.2003, found 516.2000 (0.6 ppm). HPLC purity 99.5%.

### Example 158: SN39725 (1-Methyl-2-nitro-1H-imidazol-5-yl)methyl (1-cyclopentyl-3-methyl-2-oxo-2,3-dihydro-1H-imidazo[4,5-c]pyridin-6-yl)(4-methoxy-2-methylphenyl)carbamate (247)

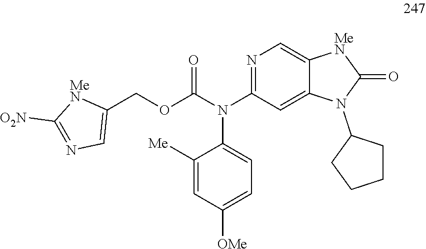 [SMILES: COc1ccc(N(C(=O)OCc2cnc([N+](=O)[O-])n2C)c2cc3c(cn2)n(C)c(=O)n3C2CCCC2)c(C)c1]

A solution of triphosgene (151 mg, 0.51 mmol) in dry DCM (5 mL) was added to a mixture of aniline 48 (179 mg, 0.51 mmol) and NaHCO₃(214 mg, 2.55 mmol) in dry DCM (20 mL) at 0° C. and the mixture was stirred at 20° C. for 24 h. A solution of (1-methyl-2-nitro-1H-imidazol-5-yl)methanol³ (160 mg, 1.02 mmol) in dry DCM (5 ml) was added and the mixture was stirred at 20° C. for 72 h. The solvent was evaporated and the residue suspended in EtOAc (50 mL) and washed with water (2×25 mL), brine (30 mL), dried (MgSO₄) and the solvent evaporated. The residue was purified by chromatography, eluting with a gradient (75-100%) of EtOAc/pet. ether, and the product was crystallised from EtOAc/pet. ether to give carbamate 247 (30 mg, 11%) as a clear gum: ¹H NMR (CDCl₃) δ 7.93 (s, 1H, H-4″), 7.52 (s, 1H, H-4), 7.16 (s, 1H, H-7), 7.09 (d, J=8.6 Hz, 1H, H-6′), 6.79 (d, J=2.9 Hz, 1H, H-3′), 6.75 (dd, J=8.5, 2.9 Hz, 1 H, H-5′), 5.21 (s, 2H, CH₂O), 4.77 (pent, J=8.7 Hz, 1H, 1″-CH), 3.80 (s, 6H, 4′-OCH₃, 1″′-CH₃), 3.39 (s, 3H, 3″-CH₃), 2.15 (s, 3H, 2′-CH₃), 2.00-2.08 (m, 4H, 2×CH₂), 1.92-1.98 (m, 2H, CH₂), 1.70-1.78 (m, 2H, CH₂); MS m/z 534.2 (MH⁺, 100%); HRMS calcd for C₂₆H₃₀N₇O₆ (MH⁺) m/z 536.2252, found 536.2254 (−0.3 ppm). HPLC purity 97.7%.

### Example 159: SN39884 (1-Methyl-2-nitro-1H-imidazol-5-yl)methyl (4-methoxy-2-methylphenyl)(3-methyl-2-oxo-1-(tetrahydro-2H-pyran-4-yl)-2,3-dihydro-1H-imidazo[4,5-c]pyridin-6-yl)carbamate (248)

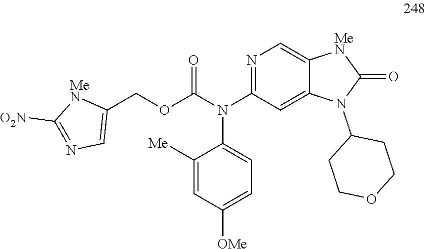 [SMILES: COc1ccc(N(C(=O)OCc2cnc([N+](=O)[O-])n2C)c2cc3c(cn2)n(C)c(=O)n3C2CCOCC2)c(C)c1]

A solution of triphosgene (193 mg, 0.65 mmol) in dry DCM (5 mL) was added to a mixture of aniline 121 (239 mg, 0.65 mmol) and NaHCO₃(273 mg, 3.25 mmol) in dry DCM (20 mL) at 0° C. and the mixture was stirred at 20° C. for 24 h. A solution of (1-methyl-2-nitro-1H-imidazol-5-yl)methanol² (204 mg, 1.30 mmol) in dry DCM (5 ml), DMAP (79 mg, 0.65 mmol) and K₂CO₃ (90 mg, 0.65 mmol) were added and the mixture was stirred at 20° C. for 72 h. The solvent was evaporated and the residue suspended in EtOAc (50 mL) and washed with water (2×25 mL), brine (30 mL), dried (MgSO₄) and the solvent evaporated. The residue was purified by chromatography, eluting with a gradient (75-100%) of EtOAc/pet. ether, and the product was crystallised from EtOAc/pet. ether to give carbamate 248 (163 mg, 45%) as a white powder: mp 142-144° C. (dec.); ¹H NMR (CDCl₃) δ 7.94 (s, 1H, H-4), 7.47 (s, 1H, H-4″′), 7.16 (s, 1H, H-7), 7.10 (d, J=8.5 Hz, 1H, H-6″), 6.78 (d, J=2.9 Hz, 1H, H-3″), 6.74 (dd, J=8.5, 2.9 Hz, 1 H, H-5″), 5.12 (s, 2H, CH₂O), 4.55 (tt, J=12.5, 4.3 Hz, 1H, 1-CH), 4.15 (br dd, J=11.5, 4.3 Hz, 2 H, H-2′, H-6′), 3.80 (s, 6H, 4″-OCH₃, 1″′-CH₃), 3.56 (br dt, J=12.0, 1.5 Hz, 2 H, H-2′, H-6′), 3.40 (s, 3H, 3-CH₃), 2.40 (dq, J=12.6, 4.6 Hz, 2 H, H-3′, H-5′), 2.15 (s, 3H, 2″-CH₃), 1.78 (br dd, J=12.5, 2.6 Hz, 2 H, H-3′, H-5′); MS m/z 552.2 (MH⁺, 100%). HRMS calcd for C₂₆H₃₀N₇O₇ (MH⁺) m/z 522.2201, found 522.2215 (−2.5 ppm). HPLC purity 99.4%.

### Example 160: SN40425 1-(1-Methyl-2-nitro-1H-imidazol-5-yl)ethyl (4-methoxy-2-methylphenyl)(3-methyl-2-oxo-1-(tetrahydro-2H-pyran-4-yl)-2,3-dihydro-1H-imidazo[4,5-c]pyridin-6-yl)carbamate (250)

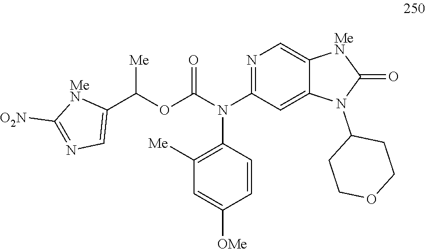 [SMILES: COc1ccc(N(C(=O)OC(C)c2cnc([N+](=O)[O-])n2C)c2cc3c(cn2)n(C)c(=O)n3C2CCOCC2)c(C)c1]

(4-Methoxy-2-methylphenyl)(3-methyl-2-oxo-1-(tetrahydro-2H-pyran-4-yl)-2,3-dihydro-1H-imidazo[4,5-c]pyridin-6-yl)carbamic chloride (249). To imidazopyridinone 121 (400 mg, 1.09 mmol) and NaHCO₃(180 mg, 2.18 mmol) in dry THF (20 mL) was added triphosgene (320 mg, 1.09 mmol) and the resulting mixture was stirred at room temperature for 24 h, then dry N₂ was bubbled through the reaction mixture for 10 minutes, water (0.2 mL) was added the mixture filtered through diatomaceous earth and solvent removed in vacuo. The product was purified by chromatography eluting with a gradient (50-100%) of EtOAc/pet. ether to give carbamic chloride 249 (250 mg, 53%) pale yellow foam which was used directly without further characterisation: ¹H NMR [(CD₃)₂SO] δ 8.17 (s, 1H, H-4), 7.86 (s, 1H, H-7), 7.44 (d, J=8.7 Hz, 1H, H-6″), 6.89 (d, J=2.8 Hz, 1H, H-3″), 6.81 (dd, J=8.7, 2.9 Hz, 1H, H-5″), 4.49 (tt, J=12.3, 4.1 Hz, 1H, H-4′), 4.01 (dd, J=12.1, 4.4 Hz, 2 H, H-2′a, H-6′a), 3.74 (s, 3 H, OCH₃), 3.49 (t, J=11.2 Hz, 2 H, H-2′b, H-6′b), 3.37 (s, 3H, 3-NCH₃), 2.44-2.31 (5H, m, 2″-CCH₃, H-3′a, H-5′a), 1.68 (dd, J=12.0, 2.3 Hz, H-3′b, H-5′b).

1-(1-methyl-2-nitro-1H-imidazol-5-yl)ethyl (4-methoxy-2-methylphenyl)(3-methyl-2-oxo-1-(tetrahydro-2H-pyran-4-yl)-2,3-dihydro-1H-imidazo[4,5-c]pyridin-6-yl)carbamate (250). To carbamoyl chloride 249 (100 mg, 0.23 mmol) in DMF (2 mL) was added 1-(1-methyl-2-nitro-1H-imidazol-5-yl)ethan-1-ol (47 mg, 0.28 mmol) and Cs₂CO₃ (90 mg, 0.28 mmol) at 0° C. and the resulting mixture was stirred at room temperature for 24 h, diluted with water (10 mL) and the resulting residues were extracted with EtOAc (3×10 mL), then the combined organic fractions were washed with water (2×10 mL), dried over Na₂SO₄ and solvent removed in vacuo. The product was purified by chromatography eluting with 1% MeOH/DCM to give carbamate 250 (24 mg, 18%) as a pale yellow solid: mp 172-175° C.; ¹H NMR [(CD₃)₂SO] δ 8.06 (s, 1H, H-4), 7.61 (s, 1H, H-7), 7.19 (d, J=8.7 Hz, H-6″), 7.14 (s, 1H, H-4″′), 6.83 (d, J=2.8 Hz, H-3″), 6.74 (dd, J=8.6, 2.9 Hz, H-5″), 6.04 (q, J=6.6 Hz, 5″′-CCH), 4.50-4.38 (m, 1H, H-4′), 4.05-3.93 (m, 2H, H-3′a, H-5′a), 3.84 (s, 3H, 1″′-NCH₃), 3.73 (s, 3H, 4″-OCH₃), 3.54-3.41 (m, 3H, H-3′b, H-5′b), 3.31 (s, 3H, 3-NCH₃), 2.39-2.25 (m, 2 H, H-2′a, H-6′a), 2.21 (s, 3H, 2″-CCH₃), 1.70-1.61 (m, 2H, H-2′b, H-6′b), 1.58 (d, J=6.6 Hz, 3 H, 5″′-CCH(CH₃). HRMS calcd for C₂₇H₃2N₇O₇ (MH⁺) m/z 566.2358, 566.2350 (−1.32 ppm). HPLC purity 97.8%

### Example 161: SN40353 (1-Methyl-5-nitro-1H-imidazol-2-yl)methyl (4-methoxy-2-methylphenyl)(3-methyl-2-oxo-1-(tetrahydro-2H-pyran-4-yl)-2,3-dihydro-1H-imidazo[4,5-c]pyridin-6-yl)carbamate (251)

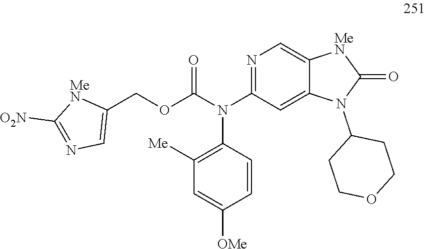 [SMILES: COc1ccc(N(C(=O)OCc2cnc([N+](=O)[O-])n2C)c2cc3c(cn2)n(C)c(=O)n3C2CCOCC2)c(C)c1]

To carbamoyl chloride 249 (30 mg, 0.07 mmol) in DMF (3 mL) was added (1-methyl-5-nitro-1H-imidazol-2-yl)methanol (13 mg, 0.084 mmol) and Cs₂CO₃ (30 mg, 0.084 mmol) and the resulting mixture stirred for 24 h, diluted with water (10 mL) and the resulting residues were extracted with EtOAc (3×10 mL), then the combined organic fractions were washed with water (2×10 mL), dried over Na₂SO₄ and solvent removed in vacuo. The product was purified by chromatography eluting with EtOAc and then triturated with Et₂O to give carbamate 251 (16 mg, 40%) as an off-white solid: mp 153-156° C.; ¹H NMR CDCl₃ δ 7.96 (s, 1H, H-4), 7.94 (s, 1H, H-4), 7.52 (s, 1H, H-7), 7.13 (d, J=8.6 Hz, 1H, H-6″), 6.78 (d, J=2.9 Hz, 1H, H-3″), 6.74 (dd, J=8.5, 2.9 Hz, 1H, H-5″), 5.29 (s, 2H, 2″-CCH₂), 4.54 (tt, J=12.4, 4.3 Hz, 1 H, H-4′), 4.15 (dd, J=11.6, 4.3 Hz, 2 H, H-2′a, H-6′a), 3.83 (s, 3H, 3″′-NCH₃), 3.80 (s, 3H, 4″-OCH₃), 3.55 (dd, J=11.9, 1.6 Hz, 2 H, H-2′b, H-6′b), 3.40 (s, 3H, 2-NCH₃), 2.44 (dq, J=12.6, 4.7 Hz, 2 H, H-3′a, H-5′a), 2.20 (s, 3H, CH₂-2″), 1.77 (dd, J=12.4, 2.6 Hz, 2 H, H-3′b, H-5′b). HRMS calcd for C₂₆H₃₀N₇O₇ (MH⁺) m/z 552.2201, found 552.2216 (+2.63 ppm). HPLC purity 99.3%

### Example 162: SN40275 (1-Methyl-2-nitro-1H-imidazol-5-yl)methyl 4-(6-((4-methoxy-2-methylphenyl)amino)-3-methyl-2-oxo-2,3-dihydro-1H-imidazo[4,5-c]pyridin-1-yl)piperidine-1-carboxylate (252)

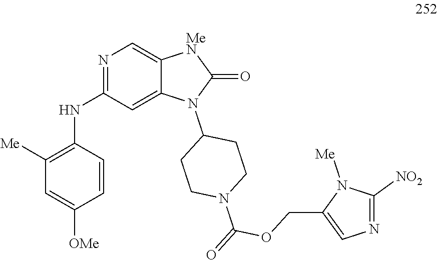 [SMILES: COc1ccc(Nc2cc3c(cn2)n(C)c(=O)n3C2CCN(C(=O)OCc3cnc([N+](=O)[O-])n3C)CC2)c(C)c1]

A solution of (1-methyl-2-nitro-1H-imidazol-5-yl)methyl (4-nitrophenyl) carbonate³ (143 mg, 0.44 mmol) in pyridine (2 mL) was added to a solution of amine 153 (136 mg, 0.37 mmol) in pyridine (5 mL) and the solution was stirred at 20° C. for 48 h. The solvent was evaporated and the residue was partitioned between EtOAc (80 mL) and water (80 mL). The organic fraction was washed sequentially with 0.1 M citric acid solution (40 mL), water (40 mL) and brine (40 mL) and dried (MgSO₄). The solvent was evaporated and the residue purified by column chromatography, eluting with a gradient (0-10%) of MeOH/EtOAc, to give carbamate 252 (52 mg, 26%) as a tan powder: ¹H NMR (CDCl₃) δ 8.14 (s, 1H, H-4′), 7.22-7.25 (m, 2H, H-6″, H-4″′), 6.83 (d, J=2.9 Hz, 1H, H-3″), 6.78 (dd, J=8.6, 2.9 Hz, 1H, H-5″), 6.09 (s, 1H, H-7), 6.03 (br s, 1H, 6′-NH), 5.18 (br s, 1H, CH₂O), 5.10 (br s, 1H, CH₂O), 4.15-4.35 (m, 3H, H-2, H-4, H-6), 4.10 (s, 3H, 1″′-CH₃), 3.81 (s, 3H, 4″-OCH₃), 3.38 (s, 3H, 3′-CH₃), 2.70-2.83 (m, 2H, H-2, H-6), 2.12-2.30 (m, 5H, H-3, H-5, 2″-CH₃), 1.70-1.82 (m, 2H, H-3, H-5); MS m/z 551.2 (MH⁺, 100%); HRMS calcd for C₂₆H₃₁N₈O₆(MH⁺) m/z 551.2361, found 551.2366 (−1.0 ppm). HPLC purity 97.6%.

### Example 163: SN40302 (1-Methyl-2-nitro-1H-imidazol-5-yl)methyl (4-(6-((4-methoxy-2-methylphenyl)amino)-3-methyl-2-oxo-2,3-dihydro-1H-imidazo[4,5-c]pyridin-1-yl)cyclohexyl)carbamate (253)

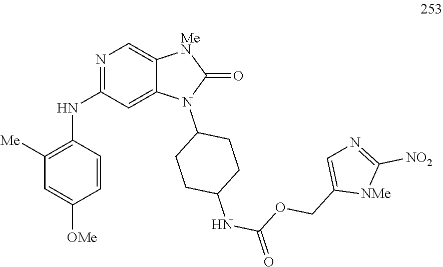 [SMILES: COc1ccc(Nc2cc3c(cn2)n(C)c(=O)n3C2CCC(NC(=O)OCc3cnc([N+](=O)[O-])n3C)CC2)c(C)c1]

A solution of (1-methyl-2-nitro-1H-imidazol-5-yl)methyl (4-nitrophenyl) carbonate⁴ (320 mg, 0.98 mmol) in pyridine (2 mL) was added to a solution of amine 185 (310 mg, 0.82 mmol) in pyridine (5 mL) and the solution was stirred at 20° C. for 24 h. The solvent was evaporated and the residue was partitioned between EtOAc (80 mL) and water (80 mL). The organic fraction was washed sequentially with 0.1 M citric acid solution (40 mL), water (40 mL) and brine (40 mL) and dried (MgSO₄). The solvent was evaporated and the residue purified by chromatography, eluting with a gradient (0-10%) of MeOH/EtOAc, to give carbamate 253 (391 mg, 84%) as a tan powder: ¹H NMR (CDCl₃) δ 7.75 (s, 1H, H-4′), 7.22 (d, J=8.6 Hz, 1H, H-6″), 7.20 (s, 1H, H-4), 6.84 (d, J=2.9 Hz, 1H, H-3″), 6.78 (dd, J=8.6, 2.9 Hz, 1H, H-5″), 6.16 (s, 1H, H-7), 6.00 (br s, 1H, 6′-NH), 5.12 (br s, 2H, CH₂O), 4.71 (br d, J=7.8 Hz, 1H, 1-NH), 4.02-4.12 (m, 4H, H-4, 1″′-CH₃), 3.83 (s, 3H, 4″-OCH₃), 3.43-3.55 (m, 1H, H-1), 3.36 (s, 3H, 3′-CH₃), 2.25 (s, 3H, 2″-CH₃), 2.07-2.17 (m, 4H, 2×CH₂), 1.79-1.88 (m, 2H, CH₂), 1.52-1.62 (m, 2H, CH₂); MS m/z 565.2 (MH⁺, 100%); HRMS calcd for C₂₇H₃₃N₈O₆ (MH⁺) m/z 565.2518, found 565.2527 (−1.6 ppm). HPLC purity 98.6%.

### Example 164: SN40527 Ethyl 7-methyl-6-((((1-methyl-2-nitro-1H-imidazol-5-yl)methoxy)carbonyl)(3-methyl-2-oxo-1-(tetrahydro-2H-pyran-4-yl)-2,3-dihydro-1H-imidazo[4,5-c]pyridin-6-yl)amino)imidazo[1,2-a]pyridine-3-carboxylate (254)

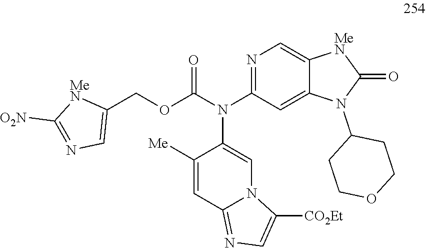 [SMILES: CCOC(=O)c1cnc2cc(C)c(N(C(=O)OCc3cnc([N+](=O)[O-])n3C)c3cc4c(cn3)n(C)c(=O)n4C3CCOCC3)cn12]

To imidazopyridinone 129 (65 mg, 0.14 mmol) and NaHCO₃(24 mg, 0.28 mmol) in dry THF (5 mL) was added triphosgene (42 mg, 0.14 mmol) and the resulting mixture was stirred at room temperature for 18 h, then dry N₂ was bubbled through the reaction mixture for 10 minutes and the resulting mixture was purified by chromatography eluting with a gradient (1-2%) of MeOH/DCM to give ethyl 6-((chlorocarbonyl)(3-methyl-2-oxo-1-(tetrahydro-2H-pyran-4-yl)-2,3-dihydro-1H-imidazo[4,5-c]pyridin-6-yl)amino)-7-methylimidazo[1,2-a]pyridine-3-carboxylate (38 mg, 54%) which was used directly without characterisation. To ethyl 6-((chlorocarbonyl)(3-methyl-2-oxo-1-(tetrahydro-2H-pyran-4-yl)-2,3-dihydro-1H-imidazo[4,5-c]pyridin-6-yl)amino)-7-methylimidazo[1,2-a]pyridine-3-carboxylate (38 mg, 0.07 mmol) in DMF (5 mL) was added (1-methyl-2-nitro-1H-imidazol-5-yl)methanol (13 mg, 0.084 mmol) and Cs₂CO₃ (27 mg, 0.084 mmol) and the resulting mixture stirred for 24 h, and then solvent was removed in vacuo. The product was purified by chromatography eluting with 1% MeOH/DCM to give carbamate 254 (6.6 mg, 17%) as an off-white solid; ¹H NMR (CDCl₃) δ 9.21 (s, 1H, H-5), 8.27 (s, 1H, H-2), 7.85 (s, 1H, H-4′), 7.66 (s, 1H, H-7), 7.60 (s, 1H, 8), 7.22 (s, 1H, 4″′), 5.26 (s, 2H, 5″′-CH₂), 4.58 (tt, J=12.4, 4.3 Hz, 1H, H-4″), 4.36 (q, J=7.1 Hz, 2 H, CH₂CH₃), 4.18 (dd, J=11.7, 4.2 Hz, 2 H, H-2″a, H-6″a), 3.82 (s, 3H, 1″′-NCH₃), 3.59 (td, J=11.9, 1.5 Hz, H-2″b, H-6″b), 3.40 (s, 3H, 3′-NCH₃), 2.54-2.38 (m, 2H, H-3″a, H-5″a), 2.27 (s, 3H, 7-CH₃), 1.81 (dd, J=12.4, 2.7 Hz, 2 H, H-3″b, H-5″b). HRMS calcd for C₂₉H₃₂N₉O₅(MH⁺) m/z 634.2368, found 634.2370 (+0.30 ppm). HPLC purity 91.0%

### Example 165: SN40536 (1-Methyl-2-nitro-1H-imidazol-5-yl)methyl (3-methyl-2-oxo-1-(tetrahydro-2H-pyran-4-yl)-2,3-dihydro-1H-imidazo[4,5-c]pyridin-6-yl)(7-methylquinoxalin-6-yl)carbamate (255)

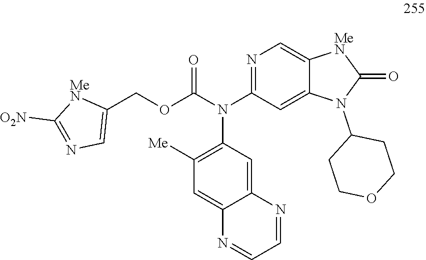 [SMILES: Cc1cc2nccnc2cc1N(C(=O)OCc1cnc([N+](=O)[O-])n1C)c1cc2c(cn1)n(C)c(=O)n2C1CCOCC1]

To imidazopyridinone 225 (50 mg, 0.13 mmol) and NaHCO₃(20 mg, 0.26 mmol) in dry THF (5 mL) was added triphosgene (40 mg, 0.13 mmol) and the resulting mixture was stirred at room temperature for 18 h, then dry N₂ was bubbled through the reaction mixture for 10 minutes and the resulting mixture was purified by chromatography eluting with a gradient (1-2%) of MeOH/DCM to give (3-methyl-2-oxo-1-(tetrahydro-2H-pyran-4-yl)-2,3-dihydro-1H-imidazo[4,5-c]pyridin-6-yl)(7-methylquinoxalin-6-yl)carbamic chloride (28 mg, 47%) which was used directly without characterisation. To (3-methyl-2-oxo-1-(tetrahydro-2H-pyran-4-yl)-2,3-dihydro-1H-imidazo[4,5-c]pyridin-6-yl)(7-methylquinoxalin-6-yl)carbamic chloride (28 mg, 0.06 mmol) in DMF (1 mL) was added (1-methyl-2-nitro-1H-imidazol-5-yl)methanol (11 m g, 0.072 mmol) and Cs₂CO₃ (23 mg, 0.072 mmol) and the resulting mixture stirred for 24 h, and then solvent was removed in vacuo. The product was purified by chromatography eluting with 0.5% MeOH/DCM to give semi-pure product which was further purified by preparative HPLC (eluting with a gradient of 45 mM NH₄CO₂H 20-98%; 90% MeCN/H₂O 80-2%) to give carbamate 255 (9.2 mg, 27%) as a white solid; ¹H NMR [(CD₃)₂SO] δ 8.93 (d, J=1.8 Hz, 1H, H-2), 8.88 (d, J=1.8 Hz, 1H, H-3), 8.04 (s, 1H, H-5), 8.03, (m, 2 H, H-4′, H-8), 7.89 (s, 1H, H-7′), 7.22 (s, 1H, H-4″′), 5.36 (s, 2H, 5″′-CCH₂), 4.79 (tt, J=12.1, 4.3 Hz, 1H, H-4″), 4.02 (dd, J=11.5, 4.2 Hz, 2 H, H-2″a, H-6″a), 3.71 (s, 3H, 1″′-NCH₃), 3.50 (t, J=11.3 Hz, 3 H, H-2″b, H-6″b), 3.30 (s, 3H, 3′-NCH₃), 2.45 (s, 3H, 7″′-CCH₃), 2.42-2.26 (m, 2H, H-3″a, H-5″a), 1.69 (br d, J=9.3 Hz, H-3″b, H-6″b). HRMS calcd for C₂₇H₂₈N₉O₆ (MH⁺) m/z 574.2157, found 574.2153 (−0.66 ppm). HPLC purity 99.4%

### Example 166: SN40537 (1-Methyl-2-nitro-1H-imidazol-5-yl)methyl (3-methyl-2-oxo-1-(tetrahydro-2H-pyran-4-yl)-2,3-dihydro-1H-imidazo[4,5-c]pyridin-6-yl)(6-methylbenzo[d][1,3]dioxol-5-yl)carbamate (256)

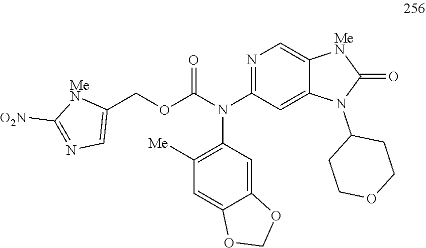 [SMILES: Cc1cc2c(cc1N(C(=O)OCc1cnc([N+](=O)[O-])n1C)c1cc3c(cn1)n(C)c(=O)n3C1CCOCC1)OCO2]

To imidazopyridinone 130 (90 mg, 0.24 mmol) and NaHCO₃(40 mg, 0.48 mmol) in dry THF (5 mL) was added triphosgene (71 mg, 0.243 mmol) and the resulting mixture was stirred at room temperature for 18 h, then dry N₂ was bubbled through the reaction mixture for 10 minutes and the resulting mixture was purified by chromatography eluting with EtOAc to give (3-methyl-2-oxo-1-(tetrahydro-2H-pyran-4-yl)-2,3-dihydro-1H-imidazo[4,5-c]pyridin-6-yl)(6-methylbenzo[d][1,3]dioxol-5-yl)carbamic chloride (57 mg, 52%) which was used directly without characterisation. To (3-methyl-2-oxo-1-(tetrahydro-2H-pyran-4-yl)-2,3-dihydro-1H-imidazo[4,5-c]pyridin-6-yl)(6-methylbenzo[d][1,3]dioxol-5-yl)carbamic chloride (40 mg, 0.09 mmol) in DMF (1 mL) was added (1-methyl-2-nitro-1H-imidazol-5-yl)methanol (17 mg, 0.108 mmol) and Cs₂CO₃ (35 mg, 0.108 mmol) and the resulting mixture stirred for 24 h, and then solvent was removed in vacuo. The product was purified by preparative HPLC (eluting with a gradient of 45 mM NH₄CO₂H 20-98%; 90% MeCN/H₂O₈-2%) to give carbamate 256 (12.9 mg, 26%) as a white solid; ¹H NMR [(CD₃)₂SO] δ 8.07 (s, 1H, H-4′), 7.67 (s, 1H, H-7), 7.21 (s, 1 H, H-4″′), 6.92 (s, 1H, H-4), 6.82 (s, 1H, H-7), 5.99 (s, 2H, 5″′-CCH₂), 5.29 (s, 2H, H-2), 4.44 (tt, J=12.2, 4.0 Hz, 1H, H-4″), 3.99 (dd, J=11.2, 4.2 Hz, 2 H, H-2″a, H-6″a), 3.76 (s, 3H, 1″′-NCH₃) 3.48 (t, J=11.3 Hz, 2 H, H-2″b, H-6″b), 3.31 (s, 3H, 3′-NCH₃), 2.40-2.23 (m, 2H, H-3″a, H-5″a), 2.10 (s, 3H, 6-CCH₃), 1.66 (dd, J=12.1, 2.5 Hz, 2 H, H-3″b, H-5″b). HRMS calcd for C₂₆H₂₅N₇O₈ (MH⁺) m/z 566.1994, found 566.1989 (−0.82 ppm). HPLC purity 99.3%

### Example 167: SN40316 (1-Methyl-2-nitro-1H-imidazol-5-yl)methyl (4-chloro-2-methylphenyl)(3-methyl-2-oxo-1-(tetrahydro-2H-pyran-4-yl)-2,3-dihydro-1H-imidazo[4,5-c]pyridin-6-yl)carbamate (257)

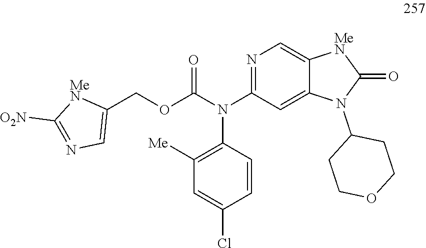 [SMILES: Cc1cc(Cl)ccc1N(C(=O)OCc1cnc([N+](=O)[O-])n1C)c1cc2c(cn1)n(C)c(=O)n2C1CCOCC1]

A solution of triphosgene (296 mg, 1.00 mmol) in dry DCM (5 mL) was added to a mixture of aniline 122 (375 mg, 1.00 mmol) and NaHCO₃(420 mg, 5.00 mmol) in dry DCM (20 mL) at 0° C. and the mixture was stirred at 20° C. for 24 h. A solution of (1-methyl-2-nitro-1H-imidazol-5-yl)methanol (237 mg, 1.51 mmol) in dry DCM (5 ml) and Cs₂CO₃ (652 mg, 2.00 mmol) were added and the mixture was stirred at 20° C. for 10 days. The solvent was evaporated and the residue suspended in EtOAc (100 mL) and washed with water (3×40 mL), brine (30 mL), dried (MgSO₄) and the solvent evaporated. The residue was purified by chromatography, eluting with a gradient (0-5%) of MeOH/DCM, and the product was crystallised from EtOAc/pet. ether to give carbamate 257 (22 mg, 4%) as a white powder: ¹H NMR (CDCl₃) δ 7.91 (s, 1H, H-4), 7.51 (s, 1H, H-4), 7.27 (m, 1H, H-3″), 7.21 (dd, J=8.4, 2.3 Hz, 1H, H-5″), 7.18 (s, 1H, H-7), 7.12 (d, J=8.4 Hz, 1H, H-6″), 5.22 (s, 2H, 5″′-CCH₂), 4.54 (tt, J=18.6, 4.3 Hz, 1H H-4′), 4.16 (dd, J=11.7, 4.3 Hz, 2 H, H-2′a, H-6′a), 3.82 (1″′-NCH₃), 3.57 (td, J=11.9, 1.0 Hz, 2 H, H-2′b, H-6′b), 3.41 (s, 3H, 3-NCH₃), 2.41 (app. qd, J=12.5, 4.6 Hz, 2 H, H-3′a, H-5′a), 2.16 (s, 3H, 2″-CCH₃), 1.78 (dd, J=12.5, 2.7 Hz, 2 H, H-3′b, H-5′b); HRMS calcd for C₂₅H₂₆ClN₇O₆ (MH⁺) 556.1733, found 556.1719 (2.5 ppm). HPLC purity 94.1%.

### Example 168: SN40317 (1-Methyl-2-nitro-1H-imidazol-5-yl)methyl (4-methoxy-2-methylphenyl)(1-(4-methoxycyclohexyl)-3-methyl-2-oxo-2,3-dihydro-1H-imidazo[4,5-c]pyridin-6-yl)carbamate (258)

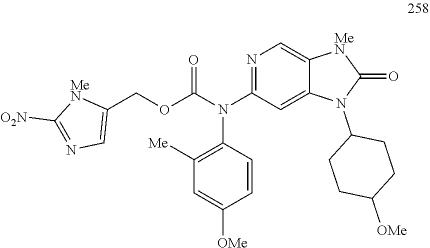 [SMILES: COc1ccc(N(C(=O)OCc2cnc([N+](=O)[O-])n2C)c2cc3c(cn2)n(C)c(=O)n3C2CCC(OC)CC2)c(C)c1]

A solution of triphosgene (243 mg, 0.82 mmol) in dry DCM (5 mL) was added to a mixture of aniline 172 (326 mg, 0.82 mmol) and NaHCO₃(344 mg, 4.10 mmol) in dry DCM (20 mL) at 0° C. and the mixture was stirred at 20° C. for 24 h. A solution of (1-methyl-2-nitro-1H-imidazol-5-yl)methanol (194 mg, 1.23 mmol) in dry DCM (5 ml) and Cs₂CO₃ (534 mg, 1.64 mmol) were added and the mixture was stirred at 20° C. for 2 days. The solvent was evaporated and the residue suspended in EtOAc (100 mL) and washed with water (3×40 mL), brine (30 mL), dried (MgSO₄) and the solvent evaporated. The residue was purified by chromatography, eluting with EtOAc and the product was crystallised from EtOAc/pet. ether to give carbamate 258 (128 mg, 27%) as a white powder: ¹H NMR (CDCl₃) δ 7.92 (s, 1H, H-4), 7.38 (s, 1H, H-4″′), 7.16 (s, 1H, H-7), 7.09 (d, J=8.6 Hz, 1H, H-6″), 6.79 (d, J=2.9 Hz, 1H, H-3″), 6.75 (dd, J=8.6, 2.9 Hz, 1H, H-5″), 5.21 (s, 2H, 5″′-CCH₂), 4.20 (tt, J=12.1, 3.9 Hz, 1H, H-1′), 3.81 (s, 3H, 1″′-NCH₃), 3.79 (s, 3H, 4″-OCH₃), 3.41 (s, 3H, 3-NCH₃), 3.39 4′-CHOCH₃), 3.29 (tt, J=10.9, 3.9 Hz, 1H, H-4′) 2.30-2.17 (m, 4H, H-2′a, H-3′a, H-5′a, H-6′a), 2.15 (s, 3H, 2″-CCH₃), 1.48-1.34 (m, 4H, H-2′b, H-3′b, H-5′b, H-6′b). HPLC purity 98.1%.

### Example 169: SN40427 (1-Methyl-2-nitro-1H-imidazol-5-yl)methyl (4-methoxy-2-methylphenyl)(1-(4-methoxyphenyl)-3-methyl-2-oxo-2,3-dihydro-1H-imidazo[4,5-c]pyridin-6-yl)carbamate (259)

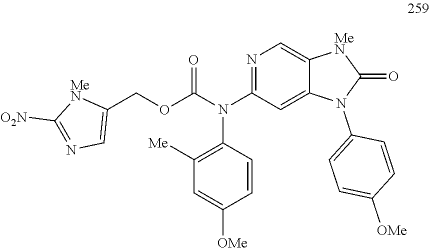 [SMILES: COc1ccc(-n2c(=O)n(C)c3cnc(N(C(=O)OCc4cnc([N+](=O)[O-])n4C)c4ccc(OC)cc4C)cc32)cc1]

A solution of triphosgene (341 mg, 1.15 mmol) in dry DCM (5 mL) was added to a mixture of aniline 195 (449 mg, 1.15 mmol) and NaHCO₃(290 mg, 3.00 mmol) in dry DCM (20 mL) at 0° C. and the mixture was stirred at 20° C. for 24 h. A solution of (1-methyl-2-nitro-1H-imidazol-5-yl)methanol (235 mg, 1.51 mmol) in dry DCM (5 ml) and Cs₂CO₃ (749 mg, 2.00 mmol) were added and the mixture was stirred at 20° C. for 2 days. The solvent was evaporated and the residue suspended in EtOAc (100 mL) and washed with water (3×40 mL), brine (30 mL), dried (MgSO₄) and the solvent evaporated. The residue was purified by chromatography, eluting with a gradient (0-5%) of MeOH/DCM, and the product was crystallised from EtOAc/pet. ether to give carbamate 259 (120 mg, 18%) as a white powder: ¹H NMR (CDCl₃) δ 8.01 (s, 1H, H-4), 7.37 (dd, J=6.8, 2.2 Hz, 2 H, H-4′, H-5′), 7.72 (s, 1H, H-4″′), 7.12 (s, 1H, H-7), 7.09-7.03 (m, 3H, H-6″, H-2′, H-5′), 6.77 (d, J=2.8 Hz, H-3″), 6.72 (dd, J=8.5, 2.9 Hz, H-5″), 5.16 (s, 2H, 5″′-CCH₂), 3.88 (s, 3H, 1″′-NCH₃), 3.79 (s, 3H, OCH₃), 3.77 (s, 3H, OCH₃), 3.47 (s, 3H, 3-NCH₃), 2.13 (s, 3H, 2″-CCH₃). HPLC purity 99.8%.

### Example 170: SN40564 N,N-Dimethyl-2-(3-methyl-4-((3-methyl-2-oxo-1-(tetrahydro-2H-pyran-4-yl)-2,3-dihydro-1H-imidazo[4,5-c]pyridin-6-yl)amino)phenoxy)-N-((1-methyl-4-nitro-1H-imidazol-5-yl)methyl)ethan-1-aminium bromide (260)

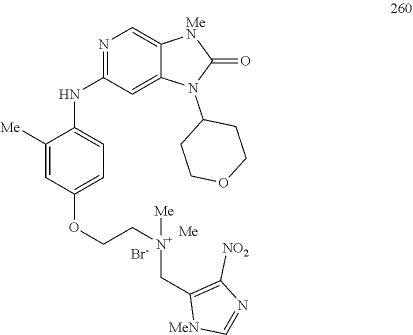 [SMILES: Cc1cc(OCC[NH+](C)Cc2c([N+](=O)[O-])ncn2C)ccc1Nc1cc2c(cn1)n(C)c(=O)n2C1CCOCC1 ; [Br-]]

To aniline 230 (100 mg, 0.24 mmol) in N-methylpyrrolidine (0.8 mL) under N₂ was added 5-(bromomethyl)-1-methyl-4-nitro-1H-imidazole (50 mg, 0.23 mmol) in N-methylpyrrolidine (0.2 mL) dropwise and the resulting mixture was stirred for 18 h. A further portion of 5-(bromomethyl)-1-methyl-4-nitro-1H-imidazole (10 mg, 0.046 mmol) in N-methylpyrrolidine (0.1 mL) was added dropwise and the solution was stirred a further 18 h. Et₂O (2 mL) was added to the reaction mixture and the N-methylpyrrolidine was decanted from the resulting amorphous gum, which was then dissolved in minimal MeCN and precipitated with a further addition of Et₂O (5 mL), then collected by filtration to give quaternary salt 260 (130 mg, 87%) as a brown solid; ¹H NMR [(CD₃)₂SO] δ 8.14 (s, 1H, H-2), 7.85 (br s, 1H, NH), 7.79 (s, 1H, H-4″), 7.56 (d, J=8.7 Hz, 1H, H-5′), 6.92 (d, J=2.1 Hz, 1H, H-2′), 6.84 (dd, J=8.7, 2.3 Hz, 1 H, 6′—H), 6.75 (s, 1H, H-7), 5.16 (br s, 2H, 5-CH₂), 4.53 (m, 2H, OCH₂CH₂N), 4.38 (tt, J=12.2, 3.7 Hz, H-4″′), 4.07-3.94 (m, 4H, H-2″′a, H-6″′a, OCH₂CH₂N), 3.88 (s, 3H, 1-NCH₃), 3.47 (t, J=11.5 Hz, 2 H, H-2″′b, H-6″′b), 3.28 (s, 3H, 3″-NCH₃), 3.19 (s, 6H, N(CH₃)₂), 2.31-2.13 (m, 5H, H-3″′a, H-5″′a, 3′-CCH₃), 1.67 (br d, J=10.0 Hz, H-3″′b, H-5″′b). HRMS calcd for C₂₈H₃₈N₈O₅ (MH⁺) m/z 565.2881, found 565.2864 (−3.14 ppm). HPLC purity 96.0%

### Example 171: Inhibition of DNA-PKcs Kinase Activity

Inhibition of DNA-PKcs kinase activity by compounds of the invention was evaluated by Reaction Biology Corporation (Malvern, PA) with the HotSpot assay platform, quantifying [γ-³³P]-ATP phosphorylation of a peptide substrate [EPPLSQEAFADLWKK, 20 μM] by human DNA-PK in the presence of 10 μg/ml DNA and 10 μM ATP. Inhibition of PI3Kα kinase activity was evaluated by Reaction Biology Corporation (Malvern, PA) using the ADP-Glo Assay quantifying the phosphorylation of 1-phosphatidyl-1D-myo-inositol 4,5-bisphosphate (10 μM) by human PI3Kα (p110α/p85α) in the presence of ATP (10 μM). Inhibition of mTOR kinase activity using the HotSpot assay platform, quantifying [γ-³³P]-ATP phosphorylation of eukaryotic translation initiation factor 4E-binding protein 1 (4EPB1, 1 μM) by human mTOR in the presence of ATP (10 μM). The results are shown in Table 12 below and entries are the mean of two replicates.

TABLE 12
Inhibition of kinase activity (as IC50)

| SN | number | DNA-PK (nM) | PICK (nM) | mTOR (nM) |
|---|---|---|---|---|
| 39228 | 7 | 4340 | 14600 | >100000 |
| 39229 | 8 | 3110 | 19300 | 77630 |
| 39231 | 9 | 3160 | 35300 | >100000 |
| 39232 | 10 | 185 | 7500 | 39400 |
| 39236 | 11 | 282 | 9620 | 68800 |
| 39239 | 12 | 6090 | 71300 | >100000 |
| 39240 | 13 | >10000 | 40400 | >100000 |
| 39241 | 14 | 565 | 36510 | >100000 |
| 39242 | 15 | 3780 | >10000 | >100000 |
| 39245 | 16 | 1750 | 74600 | >100000 |
| 39246 | 17 | >10000 | >10000 | >100000 |
| 39247 | 18 | 2000 | 36000 | 52800 |
| 39263 | 19 | >10000 | 22600 | >100000 |
| 39273 | 20 | 2490 | 17700 | 9290 |
| 39280 | 21 | 8100 | 16200 | >100000 |
| 39301 | 22 | >10000 | 43500 | >100000 |
| 39275 | 23 | 6200 | 79400 | >100000 |
| 39291 | 24 | 4030 | >10000 | >100000 |
| 39297 | 25 | >10000 | >10000 | >100000 |
| 39278 | 26 | >10000 | >10000 | >100000 |
| 39290 | 27 | 3610 | >10000 | >100000 |
| 39283 | 28 | >10000 | 57000 | >100000 |
| 39274 | 29 | 3130 | 42700 | >100000 |
| 39279 | 30 | 1090 | 8570 | 69200 |
| 39304 | 31 | 8830 | 17100 | >100000 |
| 39308 | 32 | 1220 | 9760 | 60200 |
| 39330 | 33 | >10000 | >10000 | >100000 |
| 39302 | 34 | >10000 | >10000 | >100000 |
| 39303 | 35 | >10000 | >10000 | >100000 |
| 39328 | 36 | >10000 | >10000 | >100000 |
| 39309 | 37 | 8180 | 78000 | >100000 |
| 39385 | 38 | 62.8 | 403 | 35700 |
| 39390 | 39 | 4320 | 25600 | >100000 |
| 39393 | 40 | 2380 | 37500 | >100000 |
| 39378 | 41 | 625 | 10500 | >100000 |
| 39376 | 42 | 18 | 1220 | 27900 |
| 39379 | 43 | 40 | 2060 | 68100 |
| 39396 | 44 | 137 | 1950 | 61800 |
| 39397 | 45 | 7.54 | 311 | 25900 |
| 39398 | 46 | 75 | 8040 | 51900 |
| 39401 | 47 | 5040 | ND | ND |
| 39369 | 48 | 7.33, 7.36 | 792, 1390 | 13500, 5830 |
| 39382 | 49 | 72.8 | 5390 | >100000 |
| 39474 | 50 | 411 | 3040 | ND |
| 39370 | 51 | 201 | NR | 67400 |
| 39642 | 52 | 22.4 | 8150 | ND |
| 39748 | 53 | 284 | 24000 | ND |
| 39761 | 54 | 29.8 | 3080 | ND |
| 39764 | 55 | 72.8 | 4020 | ND |
| 39673 | 56 | 100 | 89400 | ND |
| 39663 | 57 | >1000 | 12800 | ND |
| 39637 | 58 | >10000 | 16800 | ND |
| 39758 | 59 | >1000 | 2710 | ND |
| 39762 | 60 | >10000 | >10000 | ND |
| 39759 | 61 | 94 | 2700 | ND |
| 39717 | 62 | >1000 | 8210 | ND |
| 39313 | 63 | 61.4 | 1700 | 39700 |
| 39316 | 64 | 713 | 5890 | >100000 |
| 39322 | 65 | 6980 | 21800 | >100000 |
| 39323 | 66 | 2230 | 14200 | >100000 |
| 39325 | 67 | >10000 | 10000 | >100000 |
| 39326 | 68 | 298 | 6280 | 46700 |
| 39402 | 69 | 315 | 20100 | >100000 |
| 39441 | 70 | 8.9 | 14100 | ND |
| 39333 | 71 | >10000 | >10000 | >100000 |
| 39334 | 72 | 5070 | 43700 | 97700 |
| 39341 | 73 | >10000 | 48400 | >100000 |
| 39344 | 74 | >10000 | >10000 | >100000 |
| 39361 | 75 | >10000 | >10000 | >100000 |
| 39346 | 76 | 4750 | 3470 | >100000 |
| 39362 | 77 | 3250 | 3000 | 61900 |
| 39342 | 78 | 75.8 | 21100 | 14900 |
| 39360 | 79 | >10000 | 1470 | >100000 |
| 39405 | 80 | 20.2 | 3720 | 2210 |
| 39395 | 81 | 6980 | 1180 | 2020 |
| 39258 | 82 | 7600 | 20500 | 96600 |
| 39259 | 83 | 10000 | 7640 | >100000 |
| 39272 | 84 | 8100 | 27400 | >100000 |
| 39305 | 85 | 245 | 3090 | 21300 |
| 39306 | 86 | 5200 | 7570 | 92500 |
| 39329 | 87 | 10000 | 21000 | >100000 |
| 39375 | 88 | 7.2 | 429 | 1960 |
| 39777 | 94 | 108 | >100000 | ND |
| 39778 | 95 | 1930 | >100000 | ND |
| 39790 | 96 | 1060 | >100000 | ND |
| 39789 | 97 | 97 | 20800 | ND |
| 39793 | 98 | 147 | >100000 | ND |
| 39794 | 99 | 138 | 18200 | ND |
| 39478 | 104 | 138 | 3570 | >100000 |
| 39551 | 105 | 12.1 | 3260 | 71400 |
| 39887 | 110 | >1000 | 151000 | ND |
| 39878 | 115 | 6.5 | 5290 | ND |
| 39881 | 116 | 4.9 | 5460 | ND |
| 39536 | 121 | 0.34, 8.2 | 1820, 1850 | 44500, 18100 |
| 39537 | 122 | 0.39, 29.6 | 2530, 5530 | 46600, 56100 |
| 39538 | 123 | 0.13 | 678 | >100000 |
| 39871 | 124 | 422 | 5450 | ND |
| 40019 | 125 | 14.3 | 10700 | 43900 |
| 40037 | 126 | 9.06 | 4230 | 12600 |
| 40046 | 127 | 7.11 | 3960 | 5420 |
| 40050 | 128 | 1420 | >10000 | >100000 |
| 40070 | 129 | 2.57 | 314 | 19000 |
| 40092 | 130 | 3.05 | 2530 | 10800 |
| 40151 | 131 | 47.7 | 11000 | 13400 |
| 40152 | 132 | 12.6 | 5570 | 60200 |
| 39689 | 133 | >10000 | >10000 | ND |
| 39690 | 134 | >10000 | >10000 | ND |
| 39872 | 135 | 5.7,17.1 | 3220, 5240 | 2190, 32200 |
| 40071 | 136 | 3.60 | 151 | 19600 |
| 39667 | 141 | 140 | 703 | ND |
| 39550 | 146 | 1.50 | 359 | 17300 |
| 39552 | 147 | 11 | 490 | 7900 |
| 39598 | 152 | 43 | 123 | 90500 |
| 39600 | 153 | 106, 48 | 78300, 43000 | ND, >100000 |
| 39686 | 157 | 157 | 36000 | ND |
| 39627 | 162 | 150 | 187 | ND |
| 39628 | 163 | 1290 | 23700 | ND |
| 39687 | 167 | >100 | 25300 | ND |
| 39540 | 172 | 0.90, 40.4 | 1470, 1390 | 25700, 26100 |
| 39539 | 173 | 0.64 | 2170 | 27500 |
| 39581 | 178 | 32 | 2070 | 44800 |
| 39584 | 179 | 6.2 | 1380 | 16100 |
| 40297 | 184 | 53 | 1130 | 15200 |
| 39695 | 185 | 60, 21 | 17100, 8770 | ND, >100000 |
| 39623 | 190 | 5.6 | 484 | ND |
| 39519 | 195 | 0.36 | 676 | 85700 |
| 39529 | 196 | 0.27 | 237 | ND |
| 39522 | 197 | 0.09 | 33 | 33400 |
| 39520 | 198 | 23 | 1800 | ND |
| 39526 | 199 | 55 | ND | ND |
| 39530 | 200 | <0.05 | 264 | 19500 |
| 39525 | 205 | 0.94 | 26300 | ND |
| 39528 | 206 | 0.07 | 558, 441 | 38600 |
| 39547 | 211 | 0.58 | 302 | ND |
| 39548 | 212 | <0.51 | 803 | ND |
| 39546 | 213 | 1.23 | 1730 | ND |
| 39549 | 214 | 43 | ND | ND |
| 40230 | 215 | 51 | 12500 | 12300 |
| 40284 | 216 | 390 | 4620 | 42700 |
| 40249 | 217 | 52 | 33100 | 76300 |
| 40250 | 218 | 94 | 14200 | 68400 |
| 40251 | 219 | 270 | 20800 | >100000 |
| 40253 | 220 | 170 | 24000 | >100000 |
| 40252 | 221 | 441 | 9360 | >100000 |
| 40266 | 223 | 3940 | 11800 | 41200 |
| 40318 | 225 | 3.4 | 1650 | 58500 |
| 39586 | 234 | 766 | 877 | 30000 |
| 39897 | 236 | 780 | 16200 | 92900 |
| 39591 | 246 | 116 | 1280 | ND |
| 39725 | 247 | 871 | >10000 | >100000 |
| 39884 | 248 | 18.5, 140 | 107000, >100,000 | >100000, >100000 |
| 39897 | 236 | 2200 | 52300 | >100000 |
| 40353 | 251 | 1260 | >100,000 | >100000 |
| 40275 | 252 | 104 | 740 | 15600 |
| 40302 | 253 | 58 | 296 | 11400 |
| 40316 | 257 | 203 | >100,000 | >100000 |
| 40317 | 258 | 754 | 39200 | >100000 |

ND: not determined

### Example 172: Selectivity of Compound 48 for DNA-PK Compared to Other Human Kinases

Inhibition of kinase activity by compound 48 (1 μM) was evaluated by Reaction Biology Corporation (Malvern PA) with the HotSpot assay platform, against a panel of 397 protein kinases and 20 lipid kinases. The results are shown in Table 13 and FIG. 1.

TABLE 13
Inhibition of kinase activity by compound 48 against a panel of Kinase
human kinases.

| | % inhibition | | % inhibition |
|---|---|---|---|
| Kinase | at 1 μM 48* | Kinase | at 1 μM 48* |
| DNA-PK | 84.4 | NEK9 | 15.0 |
| JNK3 | 80.0 | RSK2 | 14.7 |
| JNK2 | 79.8 | CDK2/cyclin E2 | 14.1 |
| FMS | 61.0 | LCK | 13.9 |
| JNK1 | 57.3 | NEK11 | 13.7 |
| LRRK2 | 48.0 | SGK1 | 13.5 |
| TYRO3/SKY | 48.0 | CAMK1a | 13.4 |
| TNIK | 46.9 | IRR/INSRR | 13.4 |
| MELK | 42.9 | PKD2/PRKD2 | 13.3 |
| PAK3 | 42.4 | TIE2/TEK | 13.1 |
| EIF2AK3 | 38.2 | PAK1 | 12.8 |
| RSK4 | 34.2 | STK25/YSK1 | 12.4 |
| MST3/STK24 | 31.5 | BMPR2 | 12.3 |
| ERN2/IRE2 | 30.9 | CDK2/cyclin O | 12.3 |
| PKCnu/PRKD3 | 29.6 | EPHA1 | 12.2 |
| P38g | 29.0 | CAMK2g | 12.1 |
| PKCmu/PRKD1 | 28.8 | RIPK2 | 12.0 |
| IRAK4 | 26.6 | CDK2/cyclin E | 11.9 |
| MNK2 | 25.3 | PKN1/PRK1 | 11.8 |
| ARK5/NUAK1 | 21.7 | EGFR | 11.6 |
| PLK1 | 21.1 | PAK2 | 11.6 |
| PLK2 | 21.0 | KSR2 | 11.5 |
| CK1epsilon | 20.0 | CDK5/p35 | 11.4 |
| GSK3b | 19.9 | c-Src | 11.3 |
| NEK1 | 19.7 | SNRK | 11.3 |
| CDK20/cyclin T1 | 19.3 | TYK1/LTK | 11.3 |
| MYLK4 | 18.8 | TBK1 | 11.0 |
| TSSK3/STK22C | 17.8 | PKCg | 11.0 |
| ACK1 | 17.3 | PHKg1 | 11.0 |
| STK16 | 17.2 | HPK1/MAP4K1 | 10.9 |
| SLK/STK2 | 16.1 | ULK1 | 10.8 |
| IKKe/IKBKE | 15.7 | STK22D/TSSK1 | 10.8 |
| FLT3 | 15.7 | AMPK(A1/B2/G1) | 10.5 |
| CK1a1 | 15.4 | ULK2 | 10.3 |
| MAPKAPK3 | 15.0 | CAMKK2 | 10.1 |
| HCK | 15.0 | CDK5/p25 | 10.1 |
| STK33 | 10.1 | GRK5 | 7.1 |
| BRK | 10.0 | MINK/MINK1 | 7.0 |
| CDK6/cyclin D1 | 10.0 | PKN3/PRK3 | 6.9 |
| PEAK1 | 9.9 | STK32C/YANK3 | 6.9 |
| SNARK/NUAK2 | 9.7 | CK2a | 6.8 |
| BTK | 9.7 | MARK2/PAR-1Ba | 6.7 |
| ALK | 9.4 | TRKC | 6.7 |
| MASTL | 9.4 | P38a/MAPK14 | 6.6 |
| CDK2/cyclin A | 9.2 | MYO3A | 6.6 |
| EIF2AK1 | 9.2 | WNK3 | 6.4 |
| CAMK1d | 9.1 | NEK5 | 6.2 |
| AMPK(A2/B2/G3) | 9.1 | CK1d | 6.1 |
| FAK/PTK2 | 9.1 | MAPKAPK5/PRAK | 6.1 |
| CAMK1b | 9.1 | CLK1 | 6.0 |
| IKKa/CHUK | 9.0 | AXL | 6.0 |
| CDK3/cyclin E2 | 9.0 | PHKg2 | 5.9 |
| LKB1 | 8.7 | TXK | 5.9 |
| NEK7 | 8.6 | CAMK2a | 5.9 |
| STK39/STLK3 | 8.6 | Haspin | 5.7 |
| MYO3b | 8.5 | MKK6 | 5.6 |
| C-MER | 8.4 | BRSK2 | 5.6 |
| HGK/MAP4K4 | 8.4 | PAK4 | 5.5 |
| AKT2 | 8.3 | DMPK2 | 5.4 |
| MEKK6 | 8.3 | FGR | 5.4 |
| VRK1 | 8.2 | PDK1/PDPK1 | 5.1 |
| PKAcb | 8.2 | PDK4/PDHK4 | 5.1 |
| ERK7/MAPK15 | 8.2 | STK32B/YANK2 | 5.0 |
| ERN1/IRE1 | 7.9 | PKMYT1 | 4.9 |
| ERK2/MAPK1 | 7.8 | TAOK1 | 4.9 |
| ARAF | 7.8 | MLK2/MAP3K10 | 4.9 |
| MNK1 | 7.7 | BLK | 4.7 |
| MST1/STK4 | 7.7 | LATS2 | 4.7 |
| CK1g1 | 7.7 | mTOR/FRAP1 | 4.7 |
| PBK/TOPK | 7.6 | GRK3 | 4.7 |
| PKA | 7.6 | MARK4 | 4.5 |
| NLK | 7.5 | DCAMKL2 | 4.5 |
| TTBK1 | 7.5 | EPHB1 | 4.5 |
| GLK/MAP4K3 | 7.4 | VRK2 | 4.5 |
| MEK5 | 7.4 | PIM1 | 4.3 |
| EPHA2 | 7.3 | STK38/NDR1 | 4.3 |
| P38d/MAPK13 | 7.2 | NIM1 | 4.2 |
| PIM3 | 7.2 | P38b/MAPK11 | 4.0 |
| KDR/VEGFR2 | 7.2 | ROCK1 | 4.0 |
| MSK2/RPS6KA4 | 4.0 | MLCK/MYLK | 1.9 |
| EPHA7 | 3.8 | RET | 1.8 |
| CHK1 | 3.6 | YSK4/MAP3K19 | 1.8 |
| CAMK1g | 3.6 | SGK3/SGKL | 1.7 |
| NEK2 | 3.5 | Aurora B | 1.7 |
| GSK3a | 3.4 | PAK5 | 1.6 |
| MKK4 | 3.3 | BMX/ETK | 1.4 |
| EIF2AK2 | 3.2 | SRMS | 1.4 |
| IRAK1 | 3.2 | TRKB | 1.4 |
| PKCeta | 3.2 | DDR2 | 1.3 |
| TAK1 | 3.2 | DYRK2 | 1.3 |
| EIF2AK4 | 3.2 | ERK1 | 1.3 |
| EPHA6 | 3.1 | FES/FPS | 1.3 |
| CAMK2b | 3.1 | SSTK/TSSK6 | 1.2 |
| TLK1 | 3.1 | MARK1 | 1.2 |
| CDK7/cyclin H | 3.0 | CSK | 1.2 |
| TESK2 | 2.8 | FLT4/VEGFR3 | 1.1 |
| YES/YES1 | 2.8 | MSK1/RPS6KA5 | 1.1 |
| AMPK(A2/B2/G2) | 2.6 | SRPK1 | 1.1 |
| MST4 | 2.6 | ZAK/MLTK | 1.0 |
| EPHA5 | 2.5 | LYN | 1.0 |
| EPHB2 | 2.5 | OSR1/OXSR1 | 1.0 |
| DMPK | 2.5 | MLK3/MAP3K11 | 1.0 |
| MARK3 | 2.5 | TEC | 1.0 |
| DAPK1 | 2.5 | CAMK2d | 1.0 |
| TSSK2 | 2.5 | p70S6Kb/RPS6KB | 1.0 |
| MAK | 2.4 | ALK2/ACVR1 | 0.9 |
| SIK2 | 2.4 | ROS/ROS1 | 0.9 |
| PKCd | 2.3 | CHK2 | 0.9 |
| AMPK(A2/B2/G1) | 2.3 | LATS1 | 0.9 |
| CDK2/Cyclin A1 | 2.2 | COT1/MAP3K8 | 0.9 |
| STK38L/NDR2 | 2.1 | CAMK4 | 0.8 |
| PKG1a | 2.1 | PKN2/PRK2 | 0.8 |
| DYRK3 | 2.1 | FGFR2 | 0.7 |
| BRAF | 2.1 | CDC7/DBF4 | 0.7 |
| TYK2 | 2.0 | FGFR1 | 0.7 |
| PDK2/PDHK2 | 2.0 | RIPK4 | 0.7 |
| WNK1 | 2.0 | KHS/MAP4K5 | 0.7 |
| RIPK3 | 2.0 | WNK2 | 0.7 |
| EPHA4 | 1.9 | CDK1/cyclin E | 0.6 |
| PKCiota | 1.9 | DYRK1B | 0.6 |
| RON/MST1R | 1.9 | MSSK1/STK23 | 0.5 |
| TRKA | 1.9 | PASK | 0.5 |
| Aurora C | 0.5 | HIPK2 | −1.6 |
| GRK6 | 0.4 | MRCKb/CDC42BP | −1.7 |
| DDR1 | 0.4 | ABL1 | −1.8 |
| PKCzeta | 0.3 | SGK2 | −1.9 |
| CK1g2 | 0.2 | PAK6 | −2.0 |
| SIK1 | 0.2 | AKT1 | −2.1 |
| HIPK3 | 0.2 | LOK/STK10 | −2.1 |
| CDK9/cyclin T1 | 0.0 | DAPK2 | −2.1 |
| IR | 0.0 | CLK4 | −2.2 |
| CDK20/cyclin H | −0.1 | EPHB3 | −2.2 |
| FRK/PTK5 | −0.2 | EPHA8 | −2.3 |
| SBK1 | −0.2 | GRK4 | −2.3 |
| Aurora A | −0.2 | TRPM7/CHAK1 | −2.3 |
| FER | −0.2 | CTK/MATK | −2.5 |
| TNK1 | −0.3 | DYRK4 | −2.5 |
| CDK9/cyclin T2 | −0.3 | GRK7 | −2.7 |
| MAPKAPK2 | −0.3 | PKCtheta | −2.8 |
| PIM2 | −0.4 | IKKb/IKBKB | −2.9 |
| CDK8/cyclin C | −0.4 | c-Kit | −3.0 |
| CDK19/cyclin C | −0.5 | SRPK2 | −3.2 |
| PKCa | −0.6 | NEK4 | −3.3 |
| CDK4/cyclin D3 | −0.6 | CDK1/cyclin B | −3.4 |
| FYN | −0.8 | CK2a2 | −3.4 |
| CLK3 | −0.8 | JAK2 | −3.5 |
| ALK3/BMPR1A | −0.8 | TAOK3/JIK | −3.5 |
| PYK2 | −0.9 | CK1a1L | −3.7 |
| ALK4/ACVR1B | −0.9 | FGFR4 | −3.8 |
| JAK1 | −1.0 | CDK3/cyclin E | −4.1 |
| CDK6/cyclin D3 | −1.1 | ITK | −4.3 |
| NEK3 | −1.2 | NEK6 | −4.8 |
| CK1g3 | −1.2 | MEKK1 | −4.8 |
| TAOK2/TAO1 | −1.3 | PKCepsilon | −4.9 |
| MEK2 | −1.4 | MUSK | −5.1 |
| SYK | −1.4 | GRK2 | −5.1 |
| MEKK2 | −1.5 | p70S6K/RPS6KB1 | −5.1 |
| DRAK1/STK17A | −1.5 | GRK1 | −5.2 |
| C-MET | −1.5 | ERK5/MAPK7 | −5.3 |
| CDK18/cyclin Y | −1.5 | LYN B | −5.3 |
| (PCTK3) | | PDGFRa | −5.3 |
| ERBB2/HER2 | −1.5 | IGF1R | −5.3 |
| CDK9/cyclin K | −1.6 | RAF1 | −5.4 |
| JAK3 | −1.6 | ZAP70 | −5.5 |
| ULK3 | −1.6 | PKCb2 | −5.5 |
| ZIPK/DAPK3 | −5.6 | EEF2K | −8.2 |
| PDK1/PDHK1 | −5.6 | MEK3 | −8.3 |
| BRSK1 | −5.7 | MST2/STK3 | −8.4 |
| MLK4 | −5.7 | ALK1/ACVRL1 | −8.8 |
| HIPK1 | −5.7 | MKK7 | −9.1 |
| DCAMKL1 | −5.9 | DLK/MAP3K12 | −9.2 |
| STK21/CIT | −5.9 | AKT3 | −9.6 |
| TESK1 | −5.9 | MRCKa/CDC42BP | −9.7 |
| DYRK1/DYRK1A | −6.1 | EPHA3 | −10.3 |
| MEK1 | −6.1 | KSR1 | −10.3 |
| EPHB4 | −6.1 | SIK3 | −10.7 |
| TGFBR2 | −6.1 | PKG2/PRKG2 | −11.0 |
| TTBK2 | −6.1 | WEE1 | −11.7 |
| CDK17/cyclin Y | −6.2 | LCK2/ICK | −11.8 |
| (PCTK2) | | LIMK1 | −12.2 |
| RSK3 | −6.2 | FGFR3 | −12.3 |
| TLK2 | −6.4 | RSK1 | −12.3 |
| CDK1/cyclin A | −6.4 | CDK14/cyclin Y | −12.4 |
| RIPK5 | −6.4 | (PFTK1) | |
| MLCK2/MYLK2 | −6.6 | ABL2/ARG | −12.6 |
| ASK1/MAP3K5 | −6.6 | AMPK(A1/B1/G1) | −13.3 |
| LIMK2 | −6.6 | NEK8 | −13.4 |
| GCK/MAP4K2 | −6.7 | ERBB4/HER4 | −13.6 |
| ALK5/TGFBR1 | −6.7 | ROCK2 | −13.7 |
| PDK3/PDHK3 | −6.8 | PKG1b | −14.0 |
| MEKK3 | −6.9 | AMPK(A1/B1/G3) | −14.8 |
| PLK3 | −7.1 | PKCb1 | −15.4 |
| MYLK3 | −7.5 | HIPK4 | −15.6 |
| PLK4/SAK | −7.6 | AMPK(A2/B1/G1) | −15.9 |
| MLK1/MAP3K9 | −7.6 | ALK6/BMPR1B | −16.7 |
| FLT1/VEGFR1 | −7.8 | AMPK(A1/B1/G2) | −18.1 |
| CDK4/cyclin D1 | −7.8 | CDK16/CYCLIN Y | −18.4 |
| PRKX | −8.1 | (PCTAIRE) | |
| PKAcg | −8.2 | CAMKK1 | −22.4 |
| CLK2 | −8.2 | PDGFRb | −26.1 |

### Example 173: Selectivity of Compound 121 for DNA-PK Compared to Other Human Kinases

Inhibition of kinase activity by compound 121 (1 μM) was evaluated by Reaction Biology Corporation (Malvern PA) with the HotSpot assay platform, against a panel of 397 protein kinases and 20 lipid kinases. The results are shown in Table 14 and FIG. 2.

TABLE 14
Inhibition of kinase activity by compound 121 against a panel of
human kinases.

| | % | | % |
|---|---|---|---|
| | inhibition at | | inhibition at |
| Kinase | 1 μM 121* | Kinase | 1 μM121* |
| DNA-PK | 88.96 | COT1/MAP3K8 | 14.55 |
| PIM1 | 49.88 | CDK2/CYCLIN A1 | 14.45 |
| PKN1/PRK1 | 41.73 | CDK1/CYCLIN E | 14.24 |
| PI3Ka (p110a/p85a) | 38.78 | ERK2/MAPK1 | 14.06 |
| JNK2 | 37.65 | CDK2/CYCLIN E2 | 14.00 |
| PI3K (p110a(E542K)/p85a) | 36.74 | MEKK2 | 13.98 |
| PI3K (p110a(H1047R)/p85a) | 34.78 | ERK7/MAPK15 | 13.84 |
| JNK3 | 34.42 | MST2/STK3 | 13.60 |
| DDR2 | 32.53 | PIP5K1C | 13.53 |
| WEE1 | 30.95 | EPHB3 | 13.52 |
| MEKK3 | 29.67 | LRRK2 | 13.46 |
| PI3Kd (p110d/p85a) | 28.50 | SNRK | 13.46 |
| EIF2AK1 | 27.17 | NEK11 | 12.98 |
| SSTK/TSSK6 | 26.68 | ERN1/IRE1 | 12.98 |
| PI3K (p110a/p65a) | 26.17 | AURORA C | 12.95 |
| PDGFRB | 25.66 | IKKA/CHUK | 12.60 |
| JNK1 | 25.47 | DDR1 | 12.57 |
| BRAF | 24.99 | MEKK1 | 12.24 |
| MLCK2/MYLK2 | 22.70 | PKCTHETA | 12.01 |
| AKT3 | 22.48 | MRCKA/CDC42BPA | 11.98 |
| PDK2/PDHK2 | 22.23 | TTBK1 | 11.87 |
| NEK9 | 21.46 | HASPIN | 11.70 |
| TESK1 | 21.34 | CAMK1B | 11.49 |
| PI3K (p110a(E545K)/p85a) | 20.96 | CK2A | 11.32 |
| EIF2AK3 | 20.06 | MAPKAPK2 | 11.30 |
| VRK2 | 19.56 | ERN2/IRE2 | 11.23 |
| STK32C/YANK3 | 19.04 | FYN | 11.14 |
| DMPK2 | 18.02 | SGK2 | 10.96 |
| TNIK | 17.95 | TSSK2 | 10.88 |
| LATS2 | 17.89 | CAMKK2 | 10.86 |
| DCAMKL2 | 17.41 | DCAMKL1 | 10.86 |
| YES/YES1 | 17.10 | EPHB4 | 10.77 |
| PI3Kg (p110g) | 17.06 | FGFR4 | 10.71 |
| C-KIT | 17.05 | PKCNU/PRKD3 | 10.62 |
| EPHA3 | 17.01 | CDK16/CYCLIN Y (PCTAIRE) | 10.61 |
| PDK3/PDHK3 | 16.76 | WNK1 | 10.59 |
| PI3Kb (p110b/p85a) | 16.76 | DYRK1/DYRK1A | 10.51 |
| NEK6 | 16.75 | NEK5 | 10.50 |
| MLK2/MAP3K10 | 16.12 | AURORA B | 10.46 |
| PI4KA | 16.12 | BMX/ETK | 10.36 |
| ALK6/BMPR1B | 15.53 | PKACB | 10.24 |
| MLK4 | 15.39 | ALK2/ACVR1 | 10.07 |
| PDK1/PDHK1 | 15.30 | PLK3 | 10.07 |
| ALK4/ACVR1B | 14.98 | TSSK3/STK22C | 10.06 |
| AKT2 | 14.71 | STK38L/NDR2 | 10.04 |
| AXL | 14.65 | MEK3 | 9.98 |
| PHKG2 | 9.96 | PAK5 | 7.35 |
| RSK3 | 9.95 | FAK/PTK2 | 7.30 |
| BLK | 9.92 | KDR/VEGFR2 | 7.30 |
| PKN3/PRK3 | 9.88 | MAST3 | 7.30 |
| OSR1/OXSR1 | 9.78 | GRK1 | 7.06 |
| STK16 | 9.76 | LATS1 | 6.96 |
| ULK3 | 9.65 | PKN2/PRK2 | 6.86 |
| PKG1A | 9.65 | MLK1/MAP3K9 | 6.79 |
| CDK5/P25 | 9.63 | CDK1/CYCLIN B | 6.77 |
| HCK | 9.59 | LKB1 | 6.76 |
| LIMK1 | 9.54 | MKK4 | 6.69 |
| C-SRC | 9.51 | LYN B | 6.66 |
| WNK3 | 9.46 | CLK2 | 6.65 |
| CDK5/P35 | 9.43 | MLK3/MAP3K11 | 6.59 |
| WNK2 | 9.38 | MSK1/RPS6KA5 | 6.54 |
| BRSK2 | 9.37 | CDK18/CYCLIN Y (PCTK3) | 6.39 |
| MEK5 | 9.23 | EPHB2 | 6.38 |
| CAMK2A | 9.22 | MARK2/PAR-1BA | 6.37 |
| GCK/MAP4K2 | 9.16 | ERK1 | 6.35 |
| NEK3 | 9.14 | PKCB2 | 6.31 |
| TESK2 | 9.11 | RIPK5 | 6.19 |
| EPHA2 | 9.01 | SRPK2 | 6.17 |
| RSK4 | 8.98 | GRK3 | 6.10 |
| CAMK2D | 8.78 | IR | 6.00 |
| AMPK(A1/B1/G2) | 8.77 | ABL2/ARG | 5.97 |
| ALK5/TGFBR1 | 8.72 | CLK4 | 5.97 |
| PKD2/PRKD2 | 8.65 | PHKG1 | 5.70 |
| CLK3 | 8.60 | TTBK2 | 5.70 |
| PASK | 8.59 | PLK1 | 5.66 |
| EPHA7 | 8.55 | TRPM7/CHAK1 | 5.60 |
| CDK2/CYCLIN O | 8.48 | GRK7 | 5.49 |
| RSK2 | 8.16 | MTOR/FRAP1 | 5.43 |
| MUSK | 8.08 | FLT1/VEGFR1 | 5.38 |
| PKCEPSILON | 7.97 | P38A/MAPK14 | 5.31 |
| JAK2 | 7.92 | CAMK2G | 5.22 |
| LCK2/ICK | 7.90 | IRR/INSRR | 5.18 |
| MLCK/MYLK | 7.88 | MRCKB/CDC42BPB | 5.16 |
| PKG2/PRKG2 | 7.88 | PAK4 | 5.10 |
| STK33 | 7.86 | GRK6 | 4.97 |
| IRAK4 | 7.79 | JAK1 | 4.95 |
| MKK7 | 7.78 | EPHA6 | 4.86 |
| EPHA1 | 7.68 | CDK14/CYCLIN Y (PFTK1) | 4.80 |
| SLK/STK2 | 7.62 | PKCETA | 4.76 |
| PAK2 | 7.60 | TIE2/TEK | 4.75 |
| CAMKK1 | 7.52 | CHK1 | 4.69 |
| CDK9/CYCLIN T2 | 7.49 | CDK4/CYCLIN D1 | 4.66 |
| BTK | 7.47 | MARK1 | 4.65 |
| CDK2/CYCLIN A | 7.44 | PKCD | 4.65 |
| PKACG | 4.50 | EGFR | 2.52 |
| CDK2/CYCLIN E | 4.48 | EEF2K | 2.43 |
| TRKB | 4.40 | PKA | 2.41 |
| FER | 4.29 | TAOK2/TAO1 | 2.36 |
| AKT1 | 4.25 | AMPK (A2/B1/G3) | 2.31 |
| ULK2 | 4.24 | GSK3B | 2.30 |
| MAPKAPK5/PRAK | 4.22 | DYRK4 | 2.29 |
| BRSK1 | 4.18 | EIF2AK2 | 2.28 |
| SNARK/NUAK2 | 4.13 | TYRO3/SKY | 2.27 |
| AMPK(A1/B1/G3) | 4.12 | PKCMU/PRKD1 | 2.26 |
| SGK1 | 4.12 | ERK5/MAPK7 | 2.19 |
| STK21/CIT | 4.11 | FGFR1 | 2.19 |
| MSSK1/STK23 | 4.10 | DYRK1B | 2.16 |
| RSK1 | 4.03 | PI4KB | 2.16 |
| TEC | 4.03 | CK2A2 | 2.14 |
| RIPK4 | 4.01 | SGK3/SGKL | 2.13 |
| ULK1 | 3.99 | YSK4/MAP3K19 | 2.13 |
| CDK17/CYCLIN Y (PCTK2) | 3.99 | TLK1 | 2.05 |
| TYK2 | 3.94 | ROCK2 | 2.04 |
| STK38/NDR1 | 3.88 | HIPK3 | 2.00 |
| MYLK4 | 3.79 | NEK2 | 1.89 |
| GRK4 | 3.74 | PDK4/PDHK4 | 1.88 |
| CDK1/CYCLIN A | 3.72 | AMPK(A1/B1/G1) | 1.79 |
| GRK5 | 3.66 | CAMK1D | 1.78 |
| IGF1R | 3.65 | CSK | 1.69 |
| ROCK1 | 3.60 | GLK/MAP4K3 | 1.65 |
| PKCZETA | 3.57 | CAMK4 | 1.60 |
| CHK2 | 3.56 | CDK9/CYCLIN T1 | 1.55 |
| RET | 3.51 | CDK19/CYCLIN C | 1.48 |
| MEK2 | 3.50 | PIK3C3/hVPS34 | 1.41 |
| FGFR2 | 3.43 | CDK6/CYCLIN D1 | 1.36 |
| MSK2/RPS6KA4 | 3.40 | MINK/MINK1 | 1.35 |
| SPHK1 | 3.31 | DYRK2 | 1.34 |
| HIPK4 | 3.20 | ITK | 1.20 |
| DAPK1 | 3.19 | VRK1 | 1.16 |
| TBK1 | 3.16 | CK1G1 | 1.15 |
| MYLK3 | 3.12 | ASK1/MAP3K5 | 1.12 |
| MARK4 | 3.03 | ZAK/MLTK | 1.12 |
| EPHA4 | 3.01 | C-MER | 1.08 |
| PYK2 | 2.94 | NEK4 | 1.04 |
| ARK5/NUAK1 | 2.85 | FLT4/VEGFR3 | 1.02 |
| AMPK(A2/B1/G1) | 2.82 | ERBB4/HER4 | 0.97 |
| PIM3 | 2.80 | CDK3/CYCLIN E | 0.97 |
| ABL1 | 2.77 | AMPK(A2/B2/G1) | 0.93 |
| IKKB/IKBKB | 2.72 | CDK6/CYCLIN D3 | 0.78 |
| TLK2 | 2.64 | P70S6K/RPS6KB1 | 0.72 |
| CDK8/CYCLIN C | 2.63 | CTK/MATK | 0.47 |
| MNK2 | 2.57 | TAOK1 | 0.40 |
| P38D/MAPK13 | 0.38 | HIPK1 | −3.00 |
| SBK1 | 0.31 | CLK1 | −3.11 |
| NLK | 0.16 | MARK3 | −3.19 |
| GSK3A | 0.13 | AMPK (A2/B1/G2) | −3.32 |
| ACK1 | 0.03 | ZIPK/DAPK3 | −3.51 |
| PKCB1 | −0.03 | EPHA5 | −3.57 |
| ARAF | −0.06 | NIM1 | −3.74 |
| PKCG | −0.08 | AMPK(A1/B2/G1) | −3.79 |
| PLK4/SAK | −0.09 | CAMK2B | −3.82 |
| CDK9/CYCLIN K | −0.27 | GRK2 | −4.19 |
| DMPK | −0.30 | STK22D/TSSK1 | −4.44 |
| IKKE/IKBKE | −0.30 | C-MET | −4.63 |
| CK1G2 | −0.37 | P38B/MAPK11 | −4.70 |
| RIPK2 | −0.38 | NEK7 | −4.84 |
| CAMK1A | −0.41 | FES/FPS | −4.96 |
| PI3KC2a/PIK3C2A | −0.43 | PBK/TOPK | −5.04 |
| DYRK3 | −0.44 | HPK1/MAP4K1 | −5.09 |
| RAF1 | −0.47 | PAK6 | −5.19 |
| PIM2 | −0.78 | STK25/YSK1 | −5.22 |
| MKK6 | −0.79 | IRAK1 | −5.32 |
| PRKX | −0.94 | TRKA | −5.36 |
| PKCA | −1.06 | TRKC | −5.39 |
| FRK/PTK5 | −1.22 | DAPK2 | −5.46 |
| FGR | −1.28 | P38G | −5.55 |
| LOK/STK10 | −1.30 | PAK3 | −5.60 |
| PDK1/PDPK1 | −1.43 | ROS/ROS1 | −5.68 |
| SYK | −1.54 | AMPK (A1/B2/G3) | −5.70 |
| TAK1 | −1.58 | EPHB1 | −5.73 |
| LCK | −1.63 | PAK1 | −5.81 |
| AMPK (A1/B2/G2) | −1.64 | STK32B/YANK2 | −5.91 |
| ALK1/ACVRL1 | −1.66 | PKG1B | −6.04 |
| TNK1 | −1.66 | MNK1 | −6.06 |
| FMS | −1.68 | CK1A1L | −6.08 |
| CK1EPSILON | −1.74 | P70S6KB/RPS6KB2 | −6.10 |
| FGFR3 | −1.80 | SIK2 | −6.13 |
| ALK3/BMPR1A | −1.80 | TYK1/LTK | −6.39 |
| PDGFRA | −1.82 | ERBB2/HER2 | −6.45 |
| TXK | −1.92 | PI4K2A | −6.50 |
| SIK3 | −1.97 | RON/MST1R | −6.52 |
| HGK/MAP4K4 | −2.13 | EIF2AK4 | −6.68 |
| MST1/STK4 | −2.25 | CDC7/DBF4 | −6.73 |
| PIP5K1A | −2.31 | CDK4/CYCLIN D3 | −6.82 |
| ZAP70 | −2.35 | CK1A1 | −6.97 |
| SRPK1 | −2.54 | AMPK(A2/B2/G2) | −7.22 |
| CK1D | −2.73 | CK1G3 | −7.38 |
| TGFBR2 | −2.82 | MASTL | −7.60 |
| LYN | −2.82 | PKCIOTA | −7.97 |
| KHS/MAP4K5 | −2.94 | AMPK(A2/B2/G3) | −8.22 |
| BRK | −8.53 | FLT3 | −12.83 |
| MEKK6 | −8.71 | HIPK2 | −12.94 |
| EPHA8 | −8.96 | KSR2 | −13.24 |
| JAK3 | −9.13 | MEK1 | −13.34 |
| AURORA A | −9.16 | NEK1 | −14.58 |
| MST4 | −9.51 | MYO3A | −15.08 |
| BMPR2 | −10.14 | SRMS | −15.33 |
| MAK | −10.22 | DRAK1/STK17A | −16.07 |
| STK39/STLK3 | −10.49 | PLK2 | −16.13 |
| PKMYT1 | −10.89 | MAPKAPK3 | −18.39 |
| LIMK2 | −10.93 | SIK1 | −19.21 |
| MYO3B | −11.45 | KSR1 | −23.33 |
| MST3/STK24 | −11.87 | CAMK1G | −24.50 |
| CDK7/CYCLIN H | −11.91 | CDK3/CYCLIN E2 | −26.16 |
| TAOK3/JIK | −12.06 | ALK | −27.32 |
| MELK | −12.60 | SPHK2 | −35.66 |

*Relative to DMSO control

### Example 174: Compound Selectivity for PIKK Kinases

Inhibition of DNA-PKcs kinase activity by compounds of the invention was evaluated by Reaction Biology Corporation (Malvern, PA) with the HotSpot assay platform, quantifying [γ-³³P]-ATP phosphorylation of a peptide substrate [EPPLSQEAFADLWKK, 20 μM] by human DNA-PK in the presence of 10 μg/ml DNA and 10 μM ATP. Inhibition of mTOR kinase activity was evaluated using the HotSpot assay platform, quantifying [γ-³³P]-ATP phosphorylation of eukaryotic translation initiation factor 4E-binding protein 1 (4EPB1, 1 μM) by human mTOR in the presence of ATP (10 μM). Inhibition of ATM kinase activity was evaluated using ATM Human PIKK Kinase Enzymatic ELISA/EIA Assay, Kinase Profiler by Eurofins Discovery (Cerep SA) at [Km ATP]. Inhibition of ATR was evaluated using ATR/ATRIP Human PIKK Kinase Enzymatic Radiometric Assay [Km ATP], KinaseProfiler by Eurofins Discovery (Cerep SA) at [Km ATP]. The results are shown in Table 15.

TABLE 15
Inhibition of PIKK kinase activity

| | | DNA-PK | mTOR/FRAP | | |
|---|---|---|---|---|---|
| | No. | (nM)^{a} | (nM)ª | ATM (nM)^{b} | ATR (nM)^{b} |
| 39236 | 11 | 282 | 68830 | 76%^{c} | |
| 39309 | 37 | 8179 | >100000 | 56%^{c} | |
| 39369 | 48 | 7.33 | 13500 | 4046 | >100000 |
| 39673 | 56 | 100 | | 86%^{c} | |
| 39313 | 63 | 61 | 39710 | 719 | |
| 39316 | 64 | 713 | >100000 | 1105 | |
| 39322 | 65 | 6979 | >100000 | 93%^{c} | |
| 39323 | 66 | 2227 | >100000 | 92%^{c} | |
| 39325 | 67 | 10000 | >100000 | 88%^{c} | |
| 39326 | 68 | 298 | 46960 | 940 | |
| 39441 | 73 | 8.9 | | 275 | |
| 39778 | 95 | 1930 | | 94%^{c} | |
| 39790 | 96 | 1057 | | 89%^{c} | |
| 39536 | 121 | 8.2 | 18060 | 9153 | >100000 |
| 40019 | 125 | 14.3 | 43940 | 74%^{c} | >1000 |
| 40046 | 127 | 7.11 | 5421 | 2556 | |
| 40070 | 129 | 2.57 | 18915 | 79%^{c} | |
| 39581 | 178 | 32.3 | 44795 | 69%^{c} | |
| 39547 | 211 | 0.58 | | 87%^{c} | |
| 39546 | 213 | 1.23 | | 68%^{c} | |
| 40230 | 215 | 51 | 12300 | 90%^{c} | |
| 39586 | 234 | 766 | 30025 | >100000 | >100000 |
| 39897 | 236 | 780 | 92915 | 57287 | >100000 |
| 39884 | 248 | 18.5 | 10000 | >100000 | >100000 |
| AZD7648 | | 1.56 | 641 | | |

^{a}HotSpot assay Reaction Biology Corp.
^{b}KinaseProfiler by Eurofins Discovery (Cerep SA).
^{c}Percent of control at 1 μM.

### Example 175: Radiosensitisation of Human Head and Neck Squamous Cell Carcinoma Cells (HNSCC)

The ability of the compounds of the invention to radiosensitise human tumour cells was evaluated under oxic conditions using a proliferative endpoint. UT-SCC-54C cells were cultured at 37° C. in a humidified incubator with 5% CO₂ in minimal essential media (MEM) supplemented with 10% fetal calf serum (FCS), 4.5 mg/mL D-glucose, 20 mM HEPES, and 100 U/mL penicillin and 100 μg/mL streptomycin. Cells were seeded in 96-well plates at two cell densities: 200 and 800 cells per well for 0 Gy and 3 Gy radiation doses, respectively. Cells were incubated for 24 h to allow for attachment and then were treated with compounds at a range of concentrations (10 μM, 3 μM, 1 μM, 0.333 μM, 0.111 μM, and 0.037 μM) with a final DMSO concentration of 0.5%. The cells were incubated with the compounds for 1 h, then irradiated with 0 Gy or 3 Gy (Cobalt-60 Eldorado 78, 2.5 Gy/min). Cells were then incubated with the compounds for a further 24 h before wash-out with PBS and wells were replenished with fresh medium. Cells were left in the incubator for 5 days for regrowth before being fixed with 10% trichloroacetic acid, stained with 0.4% sulforhodamine B, and solubilised with 10 mM unbuffered Tris. Plates were read on a BioTek plate reader at 490 nm and 450 nm wavelengths. Blank values (no cells) were also subsequently subtracted. All values were first normalised to seeded cell numbers before being normalised against control values (0.5% DMSO only). IC50 values for 50% inhibition of regrowth fraction for compounds were calculated by logistic regression using GraphPad Prism (FIG. 3 and Table 4). S50 values for 50% inhibition of regrowth fraction for compound in combination with 3 Gy of radiation were calculated by logistic regression using GraphPad Prism (FIG. 3 and Table 16).

TABLE 16
Radiosensitisation of UT-SCC-54C HNSCC cells.

| | num- | IC50 | S50 | | num- | IC50 | S50 |
|---|---|---|---|---|---|---|---|
| SN | ber | (μM) | (μM) | SN | ber | (μM) | (μM) |
| 39376 | 42 | >10 | 1.7 | 39584 | 179 | >10 | 0.21 |
| 39379 | 43 | >10 | 5.17 | 40297 | 184 | >10 | 6.3 |
| 39397 | 45 | >10 | 0.4 | 39695 | 185 | >10 | 0.31 |
| 39369 | 48 | >10 | 1.09 | 39623 | 190 | >10 | 0.4 |
| 39762 | 60 | 6.213 | 3.22 | 39519 | 195 | >10 | 0.22 |
| 39441 | 70 | >10 | 2.62 | 39529 | 196 | >10 | 0.68 |
| 39342 | 78 | >10 | 4.09 | 39520 | 198 | >10 | 0.17 |
| 39375 | 88 | >10 | 0.68 | 39530 | 200 | >10 | 0.31 |
| 39777 | 94 | >10 | 3.12 | 39525 | 205 | >10 | 0.93 |
| 39794 | 99 | >10 | 5.99 | 39528 | 206 | >10 | 0.21 |
| 39878 | 115 | >10 | 0.4 | 39547 | 211 | >10 | 0.37 |
| 39881 | 116 | >10 | 0.06 | 39548 | 212 | >10 | 0.51 |
| 39536 | 121 | >10 | 0.34 | 39546 | 213 | >10 | 2.22 |
| 39537 | 122 | >10 | 0.38 | 40230 | 215 | >10 | 7.5 |
| 39538 | 123 | >10 | 0.74 | 40284 | 216 | >10 | 7.4 |
| 40019 | 125 | >10 | 0.67 | 40249 | 217 | >10 | 3.9 |
| 40037 | 126 | >10 | 0.32 | 40250 | 218 | >10 | 3.1 |
| 40046 | 127 | >10 | 0.22 | 40253 | 220 | >10 | 4.4 |
| 40070 | 129 | >10 | 0.09 | 40318 | 225 | >10 | 0.27 |
| 40092 | 130 | >10 | 0.50 | 39586 | 234 | 10.6 | 10.9 |
| 40151 | 131 | >10 | 4.8 | 39897 | 236 | >10 | >10 |
| 40152 | 132 | >10 | 0.49 | 40458 | 238 | >10 | >10 |
| 39872 | 135 | >10 | 0.93 | 40459 | 240 | >10 | >10 |
| 39667 | 141 | >10 | 7.41 | 39591 | 246 | >10 | 6.08 |
| 39550 | 146 | >10 | 0.83 | 39725 | 247 | >10 | >10 |
| 39552 | 147 | >10 | 0.97 | 39884 | 248 | >10 | >10 |
| 39598 | 152 | >10 | 1.71 | 40425 | 250 | >10 | >10 |
| 39600 | 153 | >10 | 4.76 | 40353 | 251 | >10 | >10 |
| 39686 | 154 | >10 | 2.06 | 40316 | 257 | >10 | >10 |
| 39540 | 172 | >10 | 0.89 | 40317 | 258 | >10 | >10 |
| 39539 | 173 | >10 | 0.85 | 40427 | 259 | >10 | >10 |
| 39581 | 178 | >10 | 1.38 | | | | |

IC50 values for 50% inhibition of regrowth fraction for compounds alone. S50 values for 50% inhibition of regrowth fraction for compound in combination with 3 Gy of radiation. IC50 values and S50 values were calculated by logistic regression using GraphPad Prism.

### Example 176: Radiosensitisation of HAP1 and HAP1/PRKDC^{-/-} Cells

The ability of the compounds of the invention to radiosensitise human tumour cells lacking functional DNA-PKcs was evaluated under oxic conditions using a proliferative endpoint. A HAP1 line with a CRISPR-induced 11 bp deletion in PRKDC at genomic location chr8:47912458 (clone HZGHC024034c011), resulting in formation of a premature termination codon truncating DNA-PKcs from 469 to 107.8 kDa (HAP1/PRKDC^{-/-}), and the parental HAP1 line (C₆₃₁), (Horizon Discovery) were cultured in Iscove's Modified Dulbecco's Medium (IMDM) with 5% FCS. The initially haploid lines were passaged for 4 weeks to allow spontaneous conversion to diploidy, which was confirmed by propidium iodide flow cytometry (Accuri C6, BD Biosciences) using HCT116 cells as a pseudodiploid reference. Cells were seeded in 96-well plates at two cell densities: 200 and 600 for HAP1 and 700 and 7000 for HAP1/PRKDC^{-/-} and were treated with compounds as described for Example 145 (See FIG. 4 and Table 17 for examples).

TABLE 17
Radiosensitisation of HAP1 and HAP1/PRKDC−/− cells.

| | | HAP1 wt | | HAP1/PRKDC−/− | |
|---|---|---|---|---|---|
| SN | number | IC50 (μM) | S50 (μM) | IC50 (μM) | S50 (μM) |
| 39369 | 48 | >10 | 2.7 | >10 | >10 |
| 39375 | 88 | >10 | 0.89 | >10 | >10 |
| 39881 | 116 | >10 | 0.58 | >10 | >10 |
| 39536 | 121 | >10 | 0.23 | >10 | >10 |
| 40019 | 125 | >10 | 0.81 | >10 | >10 |
| 40037 | 126 | >10 | 0.31 | >10 | >10 |
| 40046 | 127 | >10 | 0.25 | >10 | >10 |
| 40070 | 129 | >10 | 0.05 | >10 | >10 |
| 39872 | 135 | >10 | 1.0 | >10 | >10 |
| 39584 | 179 | >10 | 0.17 | >10 | >10 |
| 39695 | 185 | >10 | 0.47 | >10 | >10 |
| 39528 | 206 | 7.6 | 0.28 | 9.5 | 9.7 |
| 39586 | 234 | 9.742 | 9.8 | 8.6 | 9.9 |
| 39897 | 236 | >10 | >10 | >10 | >10 |
| 39884 | 248 | >10 | >10 | >10 | >10 |

IC50 values for 50% inhibition of regrowth fraction for compounds alone. S50 values for 50% inhibition of regrowth fraction for compound in combination with 3 Gy of radiation. IC50 values and S50 values were calculated by logistic regression using GraphPad Prism.

### Example 177: Inhibition of Cellular Autophosphorylation at Ser2056 of DNA-PKcs in UT-SCC-54C Cells

UT-SCC-54C cells were cultured at 37° C. in a humidified incubator with 5% CO₂ in MEM supplemented with 10% fetal calf serum, 4.5 mg/mL D-glucose+20 mM HEPES. Cells were seeded in 6-well plates at a density of 1×10⁶ cells per well and were left to attach overnight. Cells were treated with 10 μM of compound in 0.5% DMSO, and were left to incubate for 3 h before irradiation at 0 Gy or 10 Gy at a dose rate of 1.2 Gy/min. Lysate was collected 30 min from the mid-point of radiation exposure (25 minutes post-irradiation). Cell lysis was performed by aspirating existing medium and washing cells with ice-cold PBS before radioimmunoprecipitation assay (RIPA) buffer was added to each well. Wells were scraped using the back of a pipette tip and lysates were transferred into a 1.5 mL microcentrifuge tube and incubated on ice for 30 minutes with vortexing every 10 min. Samples were centrifuged at 19,000 RCF for 10 min at 4° C. and the supernatant was collected into a new tube. Protein concentrations were measured using the bicinchoninic acid assay. Protein concentrations were equalised with MilliQ water before being mixed with NuPage 4× LDS loading dye and denatured by heating at 90° C. for 10 minutes with 5% 2-mercaptoethanol. 10-15 μg of sample was loaded into each well of a NuPage 3-8% Tris-Acetate gel (Invitrogen, EA03785BOX). 10 μL of the HiMark Pre-stained ladder (ThermoFisher, LC5699) was also loaded. Gels were run at 120 V for 80 min. Proteins were transferred from the gel on to a PVDF membrane in the NuPage transfer buffer at 100 V for 60 min. Membranes were blocked with 5% BSA in TBS-Tween for 1 h before incubating with primary antibody overnight on a rocker at 4° C. Antibodies and dilutions used for immunoblotting were as follows: pDNA-PKcs 1:2,000 (Ser2016, Abcam, AB124918), Total DNA-PKcs 1:10,000 (Abcam, AB168854), pATM 1:2,000 (Ser1981 (10H₁₁.E12) Cell Signalling Technology, CST4526), β-actin 1:10,000 (Millipore, MAB1501). Bound antibodies were detected with the corresponding horseradish peroxidase (HRP)-conjugated secondary antibody, and visualised via chemiluminescence (GE Healthcare Life Sciences, GERPN2109). Images were captured using a ChemiDoc™ MP system (See FIGS. 7 and 9).

For anoxic cultures UT-SCC-54C cells were cultured at 37° C. in a humidified incubator with 5% CO₂ in MEM supplemented with 10% fetal calf serum, 4.5 mg/mL D-glucose+20 mM HEPES. Cells were trypsinised and pelleted before being transferred to a Bactron Pd/H₂-scrubbed anaerobic chamber (Sheldon Manufacturing, Cornelius, OR). Cells were seeded in 6-well plates at a density of 1×10⁶ cells per well and were left for 2 h to attach. Cells were treated with 10 μM of compound in 0.5% DMSO and were left to incubate for 3 h. Plates were sealed in a metal box to maintain the anoxic conditions before irradiation at 0 Gy or 25 Gy at a dose rate of 1.2 Gy/min at room temperature. Lysate was collected 30 min from the mid-point of radiation exposure (19.6 minutes post-irradiation) and were processed as described above (See FIG. 8).

### Example 178: Radiosensitisation of UT-SCC-54C HNSCC Cells

The ability of the compounds of the invention to radiosensitise human tumour cells was also evaluated using a clonogenic survival endpoint comparing oxic and anoxic exposure. Log-phase cells were seeded at 10⁶/mL in 96-well plates, and irradiated (Cobalt-60 Eldorado 78, 2.5 Gy/min) 5 h later using a wedge-shaped lead shield to generate a range of dose rates (0.2-1.0 Gy/min, determined using ammonium thiocyanate-modified Fricke dosimetry⁵. For anoxic drug exposures, cultures were establishing in the anaerobic chamber as above, sealed in metal boxes in the chamber, irradiated at room temperature, then immediately transferred to a standard humidified 5% CO₂ incubator at 20% 02 without changing the medium. Drug exposure was terminated 18 h after irradiation by trypsinising. Cell densities were determined with an electronic particle counter (Model Z_{F}, Coulter Beckman) and up to 5×10⁴ cells/well were plated in 6-well dishes. Colonies were stained with methylene blue 10 days later. Colonies with >50 cells were counted to determine plating efficiencies.

TABLE 18
Clonogenic survival curve parameters for radiosensitisation by
compounds under oxic and anoxic conditions, for the experiments
illustrated in FIGS. 8 and 9.

| | | | Compound | | | | | |
|---|---|---|---|---|---|---|---|---|
| | | gas | conc | Plating efficiency | D₁₀^{b} | D₁^{c} | | |
| SN | compound | phase | (μM) | drug/no drug^{a} | (Gy) | (Gy) | SER₁₀^{d} | SER₁e |
| SN39369 | 48 | 20% O₂ | 0 | 0.993 | 5.49 | 8.04 | | |
| | | | 0.33 | 0.980 | 4.54 | 6.93 | 1.21 | 1.16 |
| | | | 1 | 0.987 | 3.35 | 5.84 | 1.64 | 1.37 |
| | | | 3 | 0.993 | 2.21 | 4.52 | 2.48 | 1.78 |
| SN39536 | 121 | 20% O₂ | 0 | | 5.96 | 9.03 | | |
| | | | 0.33 | 0.93 | 4.63 | 1.29 | 7.42 | 1.22 |
| | | | 1 | 0.89 | 2.65 | 2.45 | 5.4 | 1.67 |
| | | | 3 | 1.06 | 1.73 | 3.44 | 3.85 | 2.35 |
| SN39872 | 135 | 20% O₂ | 0 | | 6.63 | 9.74 | | |
| | | | 0.33 | 0.77 | 4.85 | 1.37 | 8.09 | 1.20 |
| | | | 1 | 0.59 | 3.15 | 2.10 | 6.49 | 1.5 |
| | | | 3 | 0.63 | 2.28 | 2.91 | 5.06 | 1.92 |
| SN39519 | 195 | 20% O₂ | 0 | | 7.14 | 10.2 | | |
| | | | 0.33 | 1.08 | 4.67 | 1.53 | 7.68 | 1.33 |
| | | | 1 | 1.09 | 2.60 | 2.75 | 5.55 | 1.84 |
| | | | 3 | 1.14 | 1.83 | 3.90 | 4.05 | 2.52 |
| SN39369 | 48 | 20% O₂ | 0 | | 5.62 | 8.58 | | |
| | | | 10 | 1.10 | 1.2 | 2.82 | 4.68 | 3.04 |
| | | 0% O₂ | 0 | | 12.1 | 19.1 | | |
| | | | 10 | 0.97 | 3.52 | 6.27 | 3.44 | 1.93 |
| SN39375 | 88 | 20% O₂ | 0 | | 5.62 | 8.58 | | |
| | | | 10 | 1.13 | 1.4 | 3.01 | 4.01 | 2.85 |
| | | 0% O₂ | 0 | | 12.1 | 19.1 | | |
| | | | 10 | 1.01 | 3.52 | 4.92 | 4.53 | 2.46 |
| SN39586 | 234 | 20% O₂ | 0 | | 5.62 | 8.58 | | |
| | | | 10 | 1.57 | 4.96 | 7.43 | 1.13 | 1.16 |
| | | 0% O₂ | 0 | | 12.1 | 19.1 | | |
| | | | 10 | 1.01 | 6.78 | 9.72 | 1.79 | 1.25 |
| SN39536 | 121 | 20% O₂ | 0 | | 6.27 | 9.71 | | |
| | | | 1 | 0.99 | 2.54 | 5.45 | 2.47 | 1.78 |
| | | 0% O₂ | 0 | | 14.8 | 23.6 | | |
| | | | 1 | 1.25 | 6.18 | 11.7 | 2.39 | 2.02 |
| SN39884 | 248 | 20% O₂ | 0 | | 6.27 | 9.71 | | |
| | | | 1 | 1.21 | 5.61 | 9.34 | 1.12 | 1.04 |
| | | 0% O₂ | 0 | | 14.8 | 23.6 | | |
| | | | 1 | 1.13 | 8.57 | 13.8 | 1.73 | 1.71 |
| SN39872 | 135 | 20% O₂ | 0 | | 5.72 | 9.40 | | |
| | | | 1 | 0.89 | 2.88 | 5.93 | 1.99 | 1.59 |
| | | 0% O₂ | 0 | | 15.7 | 23.8 | | |
| | | | 1 | 1.12 | 6.37 | 11.5 | 2.47 | 2.07 |
| SN39897 | 236 | 20% O₂ | 0 | | 5.72 | 9.40 | | |
| | | | 1 | 1.05 | 6.06 | 9.41 | 0.94 | 1.00 |
| | | 0% O₂ | 0 | | 15.7 | 23.8 | | |
| | | | 1 | 1.09 | 9.96 | 17.4 | 1.58 | 1.37 |
| SN40019 | 125 | 20% O₂ | 0 | | 5.41 | | | |
| | | | 1 | 1.02 | 2.49 | 4.90 | 2.17 | 1.77 |
| SN40458 | 238 | 0% O₂ | 0 | | 13.97 | 21.58 | | |
| | | | 1 | 1.02 | 8.46 | 15.0 | 1.65 | 1.44 |
| SN40425 | 250 | 0% O₂ | 0 | | 12.06 | 20.27 | | |
| | | | 1 | 0.98 | 5.78 | 10.83 | 2.09 | 1.87 |
| SN40353 | 251 | 0% O₂ | 0 | | 13.94 | 21.46 | | |
| | | | 1 | 1.03 | 13.17 | 19.50 | 1.06 | 1.10 |

^{a}No radiation.
^{b}Radiation doses for 10% surviving fraction.
^{c}Radiation doses for 1% surviving fraction.
^{d}Sensitiser enhancement ratio at 10% surviving fraction.
^{e}Sensitiser enhancement ratio at 1% surviving fraction.

### Example 179: Radiosensitisation of UT-SCC-54C HNSCC Tumours

The activity of compounds 121 and 248 in combination with radiation was assessed using an ex vivo assay illustrated in FIG. 11. Tumour xenografts were grown on the right dorsal flank of female NIH-III mice, with body weights 18-21 g, by subcutaneous inoculation of 5×10⁶ UT-SCC-54C cells. When the tumours reached approximately 10 mm diameter, unrestrained, unanaesthetised mice were treated IP with two doses of 121 or 248 at 50 mg/kg, administered 15 min before, and 6 h after, whole-body irradiation (13 Gy, ⁶⁰Co, 1.07 Gy/min). Tumours were excised 18 h after irradiation, weighed, dissociated enzymatically and clonogens assessed by plating as previously described⁵. When administered to mice bearing UT-SCC-54C tumour xenografts, compounds 121 and 248 alone produced no reduction in the clonogens/gram of tumour tissue compared to DMSO control (FIG. 11). Radiation alone (13 Gy) produced ca. 1.5 log reduction in clonogens/gram. Administration of compound 121 produced a significant (p=0.005) additional reduction in clonogens/gram of tumour compared to radiation (13 Gy) alone. The prodrug 248 produced a smaller, but significant (p=0.018) effect compared to radiation alone. These studies demonstrate the efficacy of use of inhibitors of DNA-PK in combination with radiotherapy in an art accepted tumour xenograft model of human head and neck cancer.

### Example 180: Inhibition of UT-SCC-54C Tumour Growth

The activity of compound 121 in combination with radiation was assessed by inhibition of growth of UT-SCC-54C tumours in a tumour xenograft study illustrated in FIG. 12. Tumour xenografts were grown on the back of female NIH-III mice, with body weights 18-21 g, by subcutaneous inoculation of 5×10⁶ UT-SCC-54C cells. When the tumours reached approximately 8 mm diameter, restrained, unanaesthetised mice were treated PO with two doses of 121 at either 100 or 400 mg/kg, administered 15 min before, and 3 h after, localised irradiation (10 Gy, ⁶⁰Co, 1.07 Gy/min). When administered to mice bearing UT-SCC-54C tumour xenografts, compound 121 produced little additional tumour growth inhibition compared to DMSO control (FIG. 12A). Radiation alone (10 Gy) produced modest tumour growth inhibition. Administration of compound 121 in combination with radiation produced significant tumour growth inhibition compared to radiation alone at either 2×100 mg/kg (P=0.007) or 2×400 mg/kg (P=0.03) (FIG. 12B). These studies demonstrate the efficacy of inhibitors of DNA-PK in combination with radiotherapy in an art accepted tumour xenograft model of human head and neck cancer.

### Example 181: Hypoxia-Selective Metabolism of Prodrugs Releases DNA-PK Inhibitors

The selective metabolism of prodrug 248 by UT-SCC-54 cells under oxic and anoxic conditions was determined and is shown in FIG. 14. Trypsinised UT-SCC-54C cells (5×10⁵ cells/0.5 mL) were plated in 24-well plates. After 2 h cell attachment, 10 μM 248 was added and incubated for 0, 1, 2, or 3 h (final volume 0.5 mL, <1% DMSO). For anoxic exposures, trypsinsed cell pellets were transferred to an anaerobic chamber, resuspended in medium, previously equilibrated in the chamber for 3 d, and seeded into similarly equilibrated plates. To determine overall metabolism, extracellular medium was removed and 1 mL of ice-cold MeOH was added to extract compounds from the cells, and the MeOH extract was added to the extracellular medium. The samples are stored −80° C. until LC-MS analysis. Concentrations of 121 and 248 were quantified by LC-MS and values are means t SE from 3 biological replicates.

### 7. REFERENCES

1. Trabbic, C. J.; Overmeyer, J. H.; Alexander, E. M.; Crissman, E. J.; Kvale, H. M.; Smith, M. A.; Erhardt, P. W.; Maltese, W. A. Synthesis and Biological Evaluation of Indolyl-Pyridinyl-Propenones Having Either Methuosis or Microtubule Disruption Activity. J. Med. Chem. 2015, 58, 2489-2512.
2. Parveen, I.; Naughton, D. P.; Whish, W. J.; Threadgill, M. D. 2-nitroimidazol-5-ylmethyl as a potential bioreductively activated prodrug system: reductively triggered release of the PARP inhibitor 5-bromoisoquinolinone. Bioorg. Med. Chem. Lett. 1999, 9, 2031-2036.
3. O'Connor, L. J.; Cazares-Korner, C.; Saha, J.; Evans, C. N.; Stratford, M. R.; Hammond, E. M.; Conway, S. J. Design, synthesis and evaluation of molecularly targeted hypoxia-activated prodrugs. Nat. Protoc. 2016, 11, 781-794.
4. Hay, M. P.; Denny, W. A. Design and synthesis of imidazole methylcarbamate prodrugs of alkylating agents. Tetrahedron 2000, 56, 645-657.
5. Cross, P.; Marshall, E. S.; Baguley, B. C.; Finlay, G. J.; Matthews, J. H. L.; Wilson, W. R. Proliferative assays for the assessment of radiosensitivity of tumor cell lines using 96-well microcultures. Radiat. Oncol. Investig. 1994, 1, 261-269.

1-50. (canceled)

51: A compound of Formula I 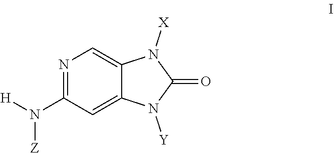
wherein:
X is selected from the group consisting of:
(a) —H,
(b) —(C₁-C₆)alkyl optionally substituted with one or more groups independently selected from —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO ₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl; and
(c) —(C₂-C₆)alkenyl optionally substituted with one or more groups independently selected from —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NRC(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl and
wherein -Ph is optionally substituted with one or more groups independently selected from —(C₁-C₆)alkyl, -halo, —OH, —OR², —NO₂, —NH₂, —NHR², —NR²R², —SH, —SR², —SO₂R²,
—SO₂NH², —CF₃, —CHF₂, —CH₂F, —CN, —CO₂H, —CO₂R², —CHO, —C(O)R², —C(O)NH₂, —C(O)NHR², and —C(O)NR²R², wherein R² is —(C₁-C₆)alkyl;
Y is selected from the group consisting of:
(a) —(C₁-C₆)alkyl optionally substituted with one or more groups
independently selected from —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkyl optionally substituted with —OH, —OR¹, —NH₂, —NHR¹ or —NR¹R¹,
and —(C₃-C₇)heterocycloalkyl which contains an oxygen or nitrogen atom in the ring and which is optionally substituted with —OH, —OR¹, —NH₂, —NHR¹, —NR¹R¹, or —(C₁-C₆)alkyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein -Ph is optionally substituted with one or more groups independently selected from —(C₁-C₆)alkyl, -halo, —OH, —OR², —NO₂, —NH₂, —NHR², —NR²R², —SH, —SR²,
—SO₂R², —SO₂NH², —CF₃, —CHF₂, —CH₂F, —CN, —CO₂H, —CO₂R², —CHO, —C(O)R₂, —C(O)NH₂, —C(O)NHR² and —C(O)NR²R², wherein R² is —(C₁-C₆)alkyl;
(b) —(C₂-C₆)alkenyl optionally substituted with one or more groups independently selected from —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkyl optionally substituted with —OH, —OR¹, —NH₂, —NHR¹ or —NR¹R¹,
and —(C₃-C₇)heterocycloalkyl which contains an oxygen or nitrogen atom in the ring and which is optionally substituted with —OH, —OR¹, —NH₂, —NHR¹, —NR¹R¹, or —(C₁-C₆)alkyl;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R₂, —C(O)NH₂, —C(O)NHR² and —C(O)NR²R², wherein R² is —(C₁-C₆)alkyl;
(c) —(C₃-C₇)cycloalkyl optionally substituted with one or more groups independently selected from —R¹, —OH, -halo, —OR¹, —OC(O)H, OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;
(d) —(C₃-C₇)heterocycloalkyl optionally substituted with one or more groups independently selected from —R¹, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl;
(e) —(C₄-C₈)aryl optionally substituted with one or more groups independently selected from —R¹, —OH, -halo, —OR¹, —OC(O)H, OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NHC(O)NR¹R¹, —NR¹C(O)NH₂, —NR¹C(O)NHR¹, —NR¹C(O)NH₂, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —O₂R², —CHO, —C(O)R², —C(O)NH₂, —C(O)NHR² and —C(O)NR²R², wherein R² is —(C₁-C₆)alkyl; and
(f) —(C₅-C₁₂)heteroaryl optionally substituted with one or more groups independently selected from —R¹, —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —N HC(O)NH₂, —NHC(O)NHR¹, —NR¹C(O)NH₂, —NHC(O)NR¹R¹, —NR¹C(O)NHR¹, —NRC(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl; and
Z is selected from the group consisting of:
(a) —(C₄-C₈)aryl optionally substituted with one or more groups independently selected from —R¹, —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, morpholinyl, piperazinyl, pyridinyl and pyrimidinyl;
wherein each R¹ is independently selected from —(C₁-C₆)alkyl and —(C₄-C₅)aryl, each of which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein each of morpholinyl, piperazinyl, pyridinyl and pyrimidinyl are optionally substituted with one or more groups selected from —(C₁-C₆)alkyl, -halo, —OH, —OR², —NO₂, —NH₂, —NHR², —NR²R², —SH, —SR², —SO₂R², —SO₂NH², —CF₃, —CHF₂, CH₂F, —CN, —CO₂H, —CO₂R², —CHO, —C(O)R², —C(O)NH₂, —C(O)NHR² and —C(O)NR²R², wherein R² is —(C₁-C₆)alkyl;
(b) —(C₅-C₁₂)heteroaryl optionally substituted with one or more groups independently selected from —R¹, —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, morpholinyl, and piperazinyl;
wherein each R¹ is independently selected from —(C₁-C₆)alkyl and —(C₄-C₈)aryl, each of which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R₂, —C(O)NH₂, —C(O)NHR² or —C(O)NR²R², wherein R² is —(C₁-C₆)alkyl, and
wherein each of morpholinyl and piperazinyl is optionally substituted with one or more group selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl.

52: The compound of claim 51, wherein X is
(b) —(C₁-C₆)alkyl optionally substituted with one or more groups independently selected from —OH, -halo, —OR¹, —OC(O)H, —OC(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO ₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, —CONR¹SO₂R¹, -Ph, —(C₃-C₇)cycloalkylamino, imidazolyl, piperazinyl, —(C₁-C₆)-alkylpiperazinyl and morpholinyl; and
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR² or —C(O)NR²R², wherein R² is —(C₁-C₆)alkyl; and
wherein -Ph is optionally substituted with one or more groups independently selected from —(C₁-C₆)alkyl, -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R₂, —C(O)NH₂, —C(O)NHR² and —C(O)NR²R², wherein R² is —(C₁-C₆)alkyl.

53: The compound of claim 51, wherein X is —(C₁-C₆)alkyl optionally substituted with OH or NH₂.

54: The compound of claim 51, wherein X is Me.

55: The compound of claim 51, wherein Y is selected from the group consisting of (c), (d) and (e) as defined in claim 51.

56: The compound of claim 51, wherein Y is selected from the group consisting of —(C₃-C₇)cycloalkyl, oxetanyl, tetrahydrofuranyl, tetrahydropyranyl, cyclohexanyl, pyrrolidinyl and piperidinyl and phenyl, each of which is optionally substituted with one or more groups independently selected from —R¹, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl.

57: The compound of claim 51, wherein Y is selected from the group consisting of —(C₃-C₇)cycloalkyl, oxetanyl, tetrahydrofuranyl, tetrahydropyranyl, methoxycyclohexanyl, hydroxycyclohexanyl, aminocyclohexanyl, N-methyl aminocyclohexanyl, N,N-dimethyl cyclohexanyl, pyrrolidinyl, N-methyl pyrrolidinyl, piperidinyl, N-methylpiperidinyl, furanyl, pyrrolyl, pyridinyl, hydroxyphenyl and methoxyphenyl.

58: The compound of claim 51, wherein Y is selected from the group consisting of optionally substituted tetrahydropyranyl, aminocyclohexanyl, hydroxycyclohexanyl, methoxycyclohexanyl, and piperidinyl.

59: The compound of claim 51, wherein Y is selected from the group consisting of 4-methoxycyclohexanyl, 4-hydroxycyclohexanyl, or 4-aminocyclohexanyl.

60: The compound of claim 51, wherein Y is selected from the group consisting of furanyl, pyrrolyl and pyridinyl.

61: The compound of claim 51, wherein Z is —(C₅-C₁₂)heteroaryl which is selected from the group consisting of furanyl, thiophenyl, pyrrolyl, pyridinyl, imidazolyl, thiazolyl, pyrimidinyl, pyrazinyl, indolyl, isoindolyl, quinolinyl, isoquinolinyl, purinyl, benzodioxolyl, quinoxalinyl, benzothiazinyl, triazolopyridinyl, benzothiazolyl, benzoxazolyl, benzodioxolyl and imidazopyridinyl, each of which may be optionally substituted with one or more groups independently selected from —R¹, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NR¹C(O)NH₂, —NHC(O)NR¹R¹, —NR¹C(O)NHR¹, —NR¹C(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl and —(C₄-C₈)aryl which is optionally substituted with -halo, —OH, —OR², —NO₂, —NH₂, —NHR², —NR²R², —SH, —SR², —SO₂R², —SO₂NH², —CF₃, —CHF₂, —CH₂F, —CN, —CO₂H, —CO₂R², —CHO, —C(O)R², —C(O)NH₂, —C(O)NHR² and —C(O)NR²R², wherein R² is —(C₁-C₆)alkyl.

62: The compound of claim 51, wherein Z is —(C₅-C₁₂)heteroaryl which is selected from the group consisting of pyrimidinyl, pyrazinyl, indolyl, isoindolyl, quinolinyl, isoquinolinyl, purinyl, benzodioxolyl, quinoxalinyl, benzothiazinyl, triazolopyridinyl, benzothiazolyl, benzoxazolyl, benzodioxolyl and imidazopyridinyl, each of which may be optionally substituted with one or more groups selected from —(C₁-C₆)alkyl, —OH, -halo, —OR¹, —OC(O)H, —C(O)R¹, —OC(O)NH₂, —OC(O)NHR¹, —O(CO)NR¹R¹, —OP(O)(OH)₂, —OP(O)(OR¹)₂, —NH₂, —NHR¹, —NR¹R¹, —NHC(O)H, —NHC(O)R¹, —NRC(O)R¹, —NHC(O)NH₂, —NHC(O)NHR¹, —NHC(O)NR¹R¹, —NRC(O)NHR¹, —NRC(O)NR¹R¹, —SH, —SR¹, —S(O)H, —S(O)R¹, —SO₂R¹, —SO₂NH₂, —SO₂NHR¹, —SO₂NR¹R¹, —CF₃, —CHF₂, —CH₂F, —CN, —CO₂H, —CO₂R¹, —CHO, —C(O)R¹, —C(O)NH₂, —C(O)NHR¹, —C(O)NR¹R¹, —CONHSO₂H, —CONHSO₂R¹, and —CONR¹SO₂R¹;
wherein each R¹ is independently selected from —(C₁-C₆)alkyl and —(C₄-C₈)aryl, each of which is optionally substituted with halo, —OH, —OR², —NO₂, —NH₂, —NHR², —NR²R², —SH, —SR², —SO₂R², —SO₂NH₂, —CF₃, —CHF₂, —CH₂F, —CN, —CO₂H, —CO₂R², —CHO, —C(O)R², —C(O)NH₂, —C(O)NHR², —C(O)NR²R² wherein R² is —(C₁-C₆)alkyl.

63: The compound of claim 51, wherein Z is —(C₅-C₁₂)heteroaryl substituted with (C₁-C₆)alkyl.

64: The compound of claim 51, wherein Z is —(C₄-C₈)aryl substituted with (C₁-C₆)alkyl.

65: The compound of claim 51, wherein Z is phenyl optionally substituted with one or more of R¹, —OH, —OR¹, -halo, —NO₂, —NH₂, —NHR¹, —NR¹R¹, —SO₂R¹ and -Bn wherein —R¹ is (C₁-C₆)alkyl.

66: The compound of claim 51, wherein Z is phenyl substituted at the 4-position with any one of —OMe, —C₁ and —OH or Z is phenyl substituted at the 5-position with one of —SO₂R¹ and —NO₂ wherein —R¹ is (C₁-C₆)alkyl.

67: The compound of claim 51, wherein Z is selected from the group consisting of 4-methoxy-2-methylphenyl, 4-chloro-2-methylphenyl, 5-(methylsulfonyl)-2-methylphenyl and 4-hydroxy-2-methylphenyl.

68: A pharmaceutical composition, comprising the compound of claim 51 or a pharmaceutically acceptable salt or solvate thereof, in combination with one or more pharmaceutically acceptable excipients.

69: A method for treating a disease in which inhibition of DNA-PK is beneficial in a subject in need thereof, the method comprising administering to the subject a therapeutically effective amount of the compound of claim 51 or a pharmaceutically acceptable salt thereof.

70: The method of claim 69, wherein the disease is cancer.
